(19) United States
(12) Patent Application Publication
(10) Pub. No.: US 2018/0002320 A1
(43) Pub. Date: Jan. 4, 2018

(54) USE OF MACROCYCLIC PICOLINAMIDES AS FUNGICIDES

(71) Applicant: DOW AGROSCIENCES LLC, Indianapolis, IN (US)

(72) Inventors: Jeremy WILMOT, Indianapolis, IN (US); Jessica HERRICK, Indianapolis, IN (US); David M. JONES, Indianapolis, IN (US); Kevin G. MEYER, Indianapolis, IN (US); Chenglin YAO, Indianapolis, IN (US); Amela ARNOLD, Indianapolis, IN (US); Johnathan E. DELORBE, Indianapolis, IN (US); Rebecca Lyn K.C. LALONDE, Indianapolis, IN (US); James M. RENGA, Indianapolis, IN (US); John F. DAEUBLE, Sr., Indianapolis, IN (US); Fangzheng LI, Indianapolis, IN (US); Karla BRAVO-ALTAMIRANO, Indianapolis, IN (US)

(21) Appl. No.: 15/540,572

(22) PCT Filed: Dec. 21, 2015
(86) PCT No.: PCT/US15/67116

§ 371 Date: Jun. 29, 2017

Related U.S. Application Data
(60) Provisional application No. 62/098,106, filed on Dec. 30, 2014.
(60) Provisional application No. 62/098,103, filed on Dec. 30, 2014.

(51) Int. Cl. C07D 405/12; A01N 43/40; A01N 43/22; A01N 43/24; C07D 321/00; C07D 313/00

(52) U.S. Cl. CPC C07D 405/12; C07D 321/00; C07D 313/00; A01N 43/22; A01N 43/24; A01N 43/40

(57) ABSTRACT

This disclosure relates to macrocyclic picolinamides of Formula I and their use as fungicides.

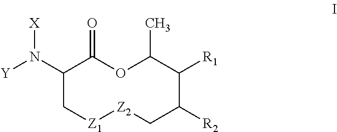

### CROSS REFERENCE TO RELATED APPLICATIONS

This application claims the benefit of U.S. Provisional Patent Application Serial Nos. 62/098,103 filed Dec. 30, 2014 and 62/098,106 filed Dec. 30, 2014, which are expressly incorporated by reference herein.

### BACKGROUND & SUMMARY

Fungicides are compounds, of natural or synthetic origin, which act to protect and/or cure plants against damage caused by agriculturally relevant fungi. Generally, no single fungicide is useful in all situations. Consequently, research is ongoing to produce fungicides that may have better performance, are easier to use, and cost less.

The present disclosure relates to macrocyclic picolinamides and their use as fungicides. The compounds of the present disclosure may offer protection against ascomycetes, basidiomycetes, deuteromycetes and oomycetes.

One embodiment of the present disclosure may include compounds of Formula I:

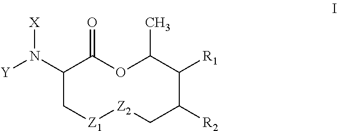

X is H or C(O)R₄;
Y is H, C(O)R₄, or Q;
Z₁ and Z₂ are independently selected from the group consisting of O and CH₂, with the proviso that Z₁ and Z₂ are not simultaneously O;
Q is

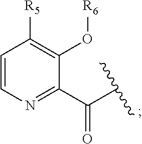

R₁ is selected from the group consisting of OR₃ and CH₂R₃;
R₂ is selected from the group consisting of OR₃, CH₂R₃, and hydrogen;
R₃ is selected from the group consisting of hydrogen, alkyl, alkenyl, aryl, Si(R₇)₃, and C(O)R₈ each optionally substituted with 0, 1, or multiple R₁₀;
R₄ is selected from the group consisting of alkyl, alkoxy, and benzyloxy, each optionally substituted with 0, 1, or multiple R₇;
R₅ is selected from the group consisting of hydrogen and alkoxy;
R₆ is selected from the group consisting of hydrogen, —C(O)R₉ and —CH₂OC(O)R₉;
R₇ is selected from the group consisting of alkyl, halo, and alkoxy;
R₈ is selected from the group consisting of alkyl, alkenyl, halo, haloalkyl, alkoxy, aryl, heteroaryl, heterocyclyl, and —C(O)R₇;
R₉ is selected from the group consisting of alkyl and alkoxy, each substituted with 0, 1, or multiple R₁₀;
R₁₀ is selected from the group consisting of alkyl, alkenyl, halo, haloalkyl, alkoxy, aryl, heteroaryl, heterocyclyl, thioalkyl, and —C(O)R₇.

Another embodiment of the present disclosure may include a fungicidal composition for the control or prevention of fungal attack comprising the compounds described above and a phytologically acceptable carrier material.

Yet another embodiment of the present disclosure may include a method for the control or prevention of fungal attack on a plant, the method including the steps of applying a fungicidally effective amount of one or more of the compounds described above to at least one of the fungus, the plant, and an area adjacent to the plant.

It will be understood by those skilled in the art that the following terms may include generic “R”-groups within their definitions, e.g., “the term alkoxy refers to an —OR substituent”. It is also understood that within the definitions for the following terms, these “R” groups are included for illustration purposes and should not be construed as limiting or being limited by substitutions about Formula I.

The term “alkyl” refers to a branched, unbranched, or saturated cyclic carbon chain, including, but not limited to, methyl, ethyl, propyl, butyl, isopropyl, isobutyl, tertiary butyl, pentyl, hexyl, cyclopropyl, cyclobutyl, cyclopentyl, cyclohexyl, and the like.

The term “alkenyl” refers to a branched, unbranched or cyclic carbon chain containing one or more double bonds including, but not limited to, ethenyl, propenyl, butenyl, isopropenyl, isobutenyl, cyclobutenyl, cyclopentenyl, cyclohexenyl, and the like.

The term “alkynyl” refers to a branched or unbranched carbon chain containing one or more triple bonds including, but not limited to, propynyl, butynyl, and the like.

The terms “aryl” and “Ar” refer to any aromatic ring, mono- or bi-cyclic, containing 0 heteroatoms.

The term “heterocyclyl” refers to any aromatic or non-aromatic ring, mono- or bi-cyclic, containing one or more heteroatoms.

The term “alkoxy” refers to an —OR substituent.

The term “acyloxy” refers to an —OC(O)R substituent.

The term “cyano” refers to a —C≡N substituent.

The term “hydroxyl” refers to an —OH substituent.

The term “amino” refers to a —N(R)₂ substituent.

The term “arylalkoxy” refers to —O(CH₂)ₙAr where n is an integer selected from the list 1, 2, 3, 4, 5, or 6.

The term “haloalkoxy” refers to an —OR—X substituent, wherein X is Cl, F, Br, or I, or any combination thereof.

The term “haloalkyl” refers to an alkyl, which is substituted with Cl, F, I, or Br or any combination thereof.

The term “halogen” or “halo” refers to one or more halogen atoms, defined as F, Cl, Br, and I.

The term “nitro” refers to a —NO₂ substituent.

The term thioalkyl refers to an —SR substituent.

Throughout the disclosure, reference to the compounds of Formula I is read as also including all stereoisomers, for example diastereomers, enantiomers, and mixtures thereof. In another embodiment, Formula (I) is read as also including salts or hydrates thereof. Exemplary salts include, but are not limited to: hydrochloride, hydrobromide, and hydroiodide.

It is also understood by those skilled in the art that additional substitution is allowable, unless otherwise noted, as long as the rules of chemical bonding and strain energy are satisfied and the product still exhibits fungicidal activity.

Another embodiment of the present disclosure is a use of a compound of Formula I, for protection of a plant against attack by a phytopathogenic organism or the treatment of a plant infested by a phytopathogenic organism, comprising the application of a compound of Formula I, or a composition comprising the compound to soil, a plant, a part of a plant, foliage, and/or roots.

Additionally, another embodiment of the present disclosure is a composition useful for protecting a plant against attack by a phytopathogenic organism and/or treatment of a plant infested by a phytopathogenic organism comprising a compound of Formula I and a phytologically acceptable carrier material.

### DETAILED DESCRIPTION

The compounds of the present disclosure may be applied by any of a variety of known techniques, either as the compounds or as formulations comprising the compounds. For example, the compounds may be applied to the roots or foliage of plants for the control of various fungi, without damaging the commercial value of the plants. The materials may be applied in the form of any of the generally used formulation types, for example, as solutions, dusts, wettable powders, flowable concentrate, or emulsifiable concentrates.

Preferably, the compounds of the present disclosure are applied in the form of a formulation, comprising one or more of the compounds of Formula I with a phytologically acceptable carrier. Concentrated formulations may be dispersed in water, or other liquids, for application, or formulations may be dust-like or granular, which may then be applied without further treatment. The formulations can be prepared according to procedures that are conventional in the agricultural chemical art.

The present disclosure contemplates all vehicles by which one or more of the compounds may be formulated for delivery and use as a fungicide. Typically, formulations are applied as aqueous suspensions or emulsions. Such suspensions or emulsions may be produced from water-soluble, water-suspendible, or emulsifiable formulations which are solids, usually known as wettable powders; or liquids, usually known as emulsifiable concentrates, aqueous suspensions, or suspension concentrates. As will be readily appreciated, any material to which these compounds may be added may be used, provided it yields the desired utility without significant interference with the activity of these compounds as antifungal agents.

Wettable powders, which may be compacted to form water-dispersible granules, comprise an intimate mixture of one or more of the compounds of Formula I, an inert carrier and surfactants. The concentration of the compound in the wettable powder may be from about 10 percent to about 90 percent by weight based on the total weight of the wettable powder, more preferably about 25 weight percent to about 75 weight percent. In the preparation of wettable powder formulations, the compounds may be compounded with any finely divided solid, such as prophyllite, talc, chalk, gypsum, Fuller's earth, bentonite, attapulgite, starch, casein, gluten, montmorillonite clays, diatomaceous earths, purified silicates or the like. In such operations, the finely divided carrier and surfactants are typically blended with the compound(s) and milled.

Emulsifiable concentrates of the compounds of Formula I may comprise a convenient concentration, such as from about 1 weight percent to about 50 weight percent of the compound, in a suitable liquid, based on the total weight of the concentrate. The compounds may be dissolved in an inert carrier, which is either a water-miscible solvent or a mixture of water-immiscible organic solvents, and emulsifiers. The concentrates may be diluted with water and oil to form spray mixtures in the form of oil-in-water emulsions. Useful organic solvents include aromatics, especially the high-boiling naphthalenic and olefinic portions of petroleum such as heavy aromatic naphtha. Other organic solvents may also be used, for example, terpenic solvents, including rosin derivatives, aliphatic ketones, such as cyclohexanone, and complex alcohols, such as 2-ethoxyethanol.

Emulsifiers which may be advantageously employed herein may be readily determined by those skilled in the art and include various nonionic, anionic, cationic and amphoteric emulsifiers, or a blend of two or more emulsifiers. Examples of nonionic emulsifiers useful in preparing the emulsifiable concentrates include the polyalkylene glycol ethers and condensation products of alkyl and aryl phenols, aliphatic alcohols, aliphatic amines or fatty acids with ethylene oxide, propylene oxides such as the ethoxylated alkyl phenols and carboxylic esters solubilized with the polyol or polyoxyalkylene. Cationic emulsifiers include quaternary ammonium compounds and fatty amine salts. Anionic emulsifiers include the oil-soluble salts (e.g., calcium) of alkylaryl sulphonic acids, oil-soluble salts or sulfated polyglycol ethers and appropriate salts of phosphated polyglycol ether.

Representative organic liquids which may be employed in preparing the emulsifiable concentrates of the compounds of the present disclosure are the aromatic liquids such as xylene, propyl benzene fractions; or mixed naphthalene fractions, mineral oils, substituted aromatic organic liquids such as dioctyl phthalate; kerosene; dialkyl amides of various fatty acids, particularly the dimethyl amides of fatty glycols and glycol derivatives such as the n-butyl ether, ethyl ether or methyl ether of diethylene glycol, the methyl ether of triethylene glycol, petroleum fractions or hydrocarbons such as mineral oil, aromatic solvents, paraffinic oils, and the like; vegetable oils such as soy bean oil, rape seed oil, olive oil, castor oil, sunflower seed oil, coconut oil, corn oil, cotton seed oil, linseed oil, palm oil, peanut oil, safflower oil, sesame oil, tung oil and the like; esters of the above vegetable oils; and the like. Mixtures of two or more organic liquids may also be employed in the preparation of the emulsifiable concentrate. Organic liquids include xylene, and propyl benzene fractions, with xylene being most preferred in some cases. Surface-active dispersing agents are typically employed in liquid formulations and in an amount of from 0.1 to 20 percent by weight based on the combined weight of the dispersing agent with one or more of the compounds. The formulations can also contain other compatible additives, for example, plant growth regulators and other biologically active compounds used in agriculture.

Aqueous suspensions comprise suspensions of one or more water-insoluble compounds of Formula I, dispersed in an aqueous vehicle at a concentration in the range from about 1 to about 50 weight percent, based on the total weight of the aqueous suspension. Suspensions are prepared by finely grinding one or more of the compounds, and vigorously mixing the ground material into a vehicle comprised of water and surfactants chosen from the same types discussed above. Other components, such as inorganic salts and synthetic or natural gums, may also be added to increase the density and viscosity of the aqueous vehicle.

The compounds of Formula I can also be applied as granular formulations, which are particularly useful for applications to the soil. Granular formulations generally contain from about 0.5 to about 10 weight percent, based on the total weight of the granular formulation of the compound(s), dispersed in an inert carrier which consists entirely or in large part of coarsely divided inert material such as attapulgite, bentonite, diatomite, clay or a similar inexpensive substance. Such formulations are usually prepared by dissolving the compounds in a suitable solvent and applying it to a granular carrier which has been preformed to the appropriate particle size, in the range of from about 0.5 to about 3 mm. A suitable solvent is a solvent in which the compound is substantially or completely soluble. Such formulations may also be prepared by making a dough or paste of the carrier and the compound and solvent, and crushing and drying to obtain the desired granular particle.

Dusts containing the compounds of Formula I may be prepared by intimately mixing one or more of the compounds in powdered form with a suitable dusty agricultural carrier, such as, for example, kaolin clay, ground volcanic rock, and the like. Dusts can suitably contain from about 1 to about 10 weight percent of the compounds, based on the total weight of the dust.

The formulations may additionally contain adjuvant surfactants to enhance deposition, wetting, and penetration of the compounds onto the target crop and organism. These adjuvant surfactants may optionally be employed as a component of the formulation or as a tank mix. The amount of adjuvant surfactant will typically vary from 0.01 to 1.0 percent by volume, based on a spray-volume of water, preferably 0.05 to 0.5 volume percent. Suitable adjuvant surfactants include, but are not limited to ethoxylated nonyl phenols, ethoxylated synthetic or natural alcohols, salts of the esters or sulphosuccinic acids, ethoxylated organosilicones, ethoxylated fatty amines, blends of surfactants with mineral or vegetable oils, crop oil concentrate (mineral oil (85%)+emulsifiers (15%)); nonylphenol ethoxylate; benzylcocoalkyldimethyl quaternary ammonium salt; blend of petroleum hydrocarbon, alkyl esters, organic acid, and anionic surfactant; C₉-C₁₁ alkylpolyglycoside; phosphated alcohol ethoxylate; natural primary alcohol (C₁₂-C₁₆) ethoxylate; di-sec-butylphenol EO-PO block copolymer; polysiloxane-methyl cap; nonylphenol ethoxylate+urea ammonium nitrate; emulsified methylated seed oil; tridecyl alcohol (synthetic) ethoxylate (8EO); tallow amine ethoxylate (15 EO); PEG (400) dioleate-99. The formulations may also include oil-in-water emulsions such as those disclosed in U.S. patent application Ser. No. 11/495,228, the disclosure of which is expressly incorporated by reference herein.

The formulations may optionally include combinations that contain other pesticidal compounds. Such additional pesticidal compounds may be fungicides, insecticides, herbicides, nematocides, miticides, arthropodicides, bactericides or combinations thereof that are compatible with the compounds of the present disclosure in the medium selected for application, and not antagonistic to the activity of the present compounds. Accordingly, in such embodiments, the other pesticidal compound is employed as a supplemental toxicant for the same or for a different pesticidal use. The compounds of Formula I and the pesticidal compound in the combination can generally be present in a weight ratio of from 1:100 to 100:1.

The compounds of the present disclosure may also be combined with other fungicides to form fungicidal mixtures and synergistic mixtures thereof. The fungicidal compounds of the present disclosure are often applied in conjunction with one or more other fungicides to control a wider variety of undesirable diseases. When used in conjunction with other fungicide(s), the presently claimed compounds may be formulated with the other fungicide(s), tank-mixed with the other fungicide(s) or applied sequentially with the other fungicide(s). Such other fungicides may include 2-(thiocyanatomethylthio)-benzothiazole, 2-phenylphenol, 8-hydroxyquinoline sulfate, ametoctradin, amisulbrom, antimycin, Ampelomyces quisqualis, azaconazole, azoxystrobin, Bacillus subtilis, Bacillus subtilis strain QST713, benalaxyl, benomyl, benthiavalicarb-isopropyl, benzovindiflupyr benzylaminobenzene-sulfonate (BABS) salt, bicarbonates, biphenyl, bismerthiazol, bitertanol, bixafen, blasticidin-S, borax, Bordeaux mixture, boscalid, bromuconazole, bupirimate, calcium polysulfide, captafol, captan, carbendazim, carboxin, carpropamid, carvone, chlazafenone, chloroneb, chlorothalonil, chlozolinate, Coniothyrium minitans, copper hydroxide, copper octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlorophen, diclocymet, diclomezine, dichloran, diethofencarb, difenoconazole, difenzoquation, diflumetorim, dimethomorph, dimoxystrobin, diniconazole, diniconazole-M, dinobuton, dinocap, diphenylamine, dithianon, dodemorph, dodemorph acetate, dodine, dodine free base, edifenphos, enestrobin, enestroburin, epoxiconazole, ethaboxam, ethoxyquin, etridiazole, famoxadone, fenamidone, fenarimol, fenbuconazole, fenfuram, fenhexamid, fenoxanil, fenpiclonil, fenpropidin, fenpropimorph, fenpyrazamine, fentin, fentin acetate, fentin hydroxide, ferbam, ferimzone, fluazinam, fludioxonil, flumorph, fluopicolide, fluopyram, fluoroimide, fluoxastrobin, fluquinconazole, flusilazole, flusulfamide, flutianil, flutolanil, flutriafol, fluxapyroxad, folpet, formaldehyde, fosetyl, fosetyl-aluminium, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), iodocarb, ipconazole, ipfenpyrazolone, iprobenfos, iprodione, iprovalicarb, isoprothiolane, isopyrazam, isotianil, kasugamycin, kasugamycin hydrochloride hydrate, kresoxim-methyl, laminarin, mancopper, mancozeb, mandipropamid, maneb, mefenoxam, mepanipyrim, mepronil, meptyl-dinocap, mercuric chloride, mercuric oxide, mercurous chloride, metalaxyl, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, nitrothal-isopropyl, nuarimol, octhilinone, ofurace, oleic acid (fatty acids), orysastrobin, oxadixyl, oxine-copper, oxpoconazole fumarate, oxycarboxin, pefurazoate, penconazole, pencycuron, penflufen, pentachlorophenol, pentachlorophenyl laurate, penthiopyrad, phenylmercury acetate, phosphonic acid, phthalide, picoxystrobin, polyoxin B, polyoxins, polyoxorim, potassium bicarbonate, potassium hydroxyquinoline sulfate, probenazole, prochloraz, procymidone, propamocarb, propamocarb hydrochloride, propiconazole, propineb, proquinazid, prothioconazole, pyraclostrobin, pyrametostrobin, pyraoxystrobin, pyrazophos, pyribencarb, pyributicarb, pyrifenox, pyrimethanil, pyriofenone, pyroquilon, quinoclamine, quinoxyfen, quintozene, Reynoutria sachalinensis extract, sedaxane, silthiofam, simeconazole, sodium 2-phenylphenoxide, sodium bicarbonate, sodium pentachlorophenoxide, spiroxamine, sulfur, SYP-Z048, tar oils, tebuconazole, tebufloquin, tecnazene, tetraconazole, thiabendazole, thifluzamide, thiophanate-methyl, thiram, tiadinil, tolclofos-methyl, tolylfluanid, triadimefon, triadimenol, triazoxide, tricyclazole, tridemorph, trifloxystrobin, triflumizole, triforine, triticonazole, validamycin, valifenalate, valiphenal, vinclozolin, zineb, ziram, zoxamide, Candida oleophila, Fusarium oxysporum, Gliocladium spp., Phlebiopsis gigantea, Streptomyces griseoviridis, Trichoderma spp., (RS)—N-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine 1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-1-enyl)phenyl thiocyanateme, ampropylfos, anilazine, azithiram, barium polysulfide, Bayer 32394, benodanil, benquinox, bentaluron, benzamacril; benzamacril-isobutyl, benzamorf, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, copper bis(3-phenylsalicylate), copper zinc chromate, cufraneb, cupric hydrazinium sulfate, cuprobam, cyclafuramid, cypendazole, cyprofuram, decafentin, dichlone, dichlozoline, diclobutrazol, dimethirimol, dinocton, dinosulfon, dinoterbon, dipyrithione, ditalimfos, dodicin, drazoxolon, EBP, ESBP, etaconazole, etem, ethirim, fenaminosulf, fenapanil, fenitropan, fluotrimazole, furcarbanil, furconazole, furconazole-cis, furmecyclox, furophanate, glyodine, griseofulvin, halacrinate, Hercules 3944, hexylthiofos, ICIA0858, isopamphos, isovaledione, mebenil, mecarbinzid, metazoxolon, methfuroxam, methylmercury dicyandiamide, metsulfovax, milneb, mucochloric anhydride, myclozolin, N-3,5-dichlorophenyl-succinimide, N-3-nitrophenylitaconimide, natamycin, N-ethylmercurio-4-toluenesulfonanilide, nickel bis(dimethyldithiocarbamate), OCH, phenylmercury dimethyldithiocarbamate, phenylmercury nitrate, phosdiphen, prothiocarb; prothiocarb hydrochloride, pyracarbolid, pyridinitril, pyroxychlor, pyroxyfur, quinacetol; quinacetol sulfate, quinazamid, quinconazole, rabenzazole, salicylanilide, SSF-109, sultropen, tecoram, thiadifluor, thicyofen, thiochlorfenphim, thiophanate, thioquinox, tioxymid, triamiphos, triarimol, triazbutil, trichlamide, urbacid, zarilamid, and any combinations thereof.

Additionally, the compounds described herein may be combined with other pesticides, including insecticides, nematocides, miticides, arthropodicides, bactericides or combinations thereof that are compatible with the compounds of the present disclosure in the medium selected for application, and not antagonistic to the activity of the present compounds to form pesticidal mixtures and synergistic mixtures thereof. The fungicidal compounds of the present disclosure may be applied in conjunction with one or more other pesticides to control a wider variety of undesirable pests. When used in conjunction with other pesticides, the presently claimed compounds may be formulated with the other pesticide(s), tank-mixed with the other pesticide(s) or applied sequentially with the other pesticide(s). Typical insecticides include, but are not limited to: 1,2-dichloropropane, abamectin, acephate, acetamiprid, acethion, acetoprole, acrinathrin, acrylonitrile, alanycarb, aldicarb, aldoxycarb, aldrin, allethrin, allosamidin, allyxycarb, alpha-cypermethrin, alpha-ecdysone, alpha-endosulfan, amidithion, aminocarb, amiton, amiton oxalate, amitraz, anabasine, athidathion, azadirachtin, azamethiphos, azinphos-ethyl, azinphos-methyl, azothoate, barium hexafluorosilicate, barthrin, bendiocarb, benfuracarb, bensultap, beta-cyfluthrin, beta-cypermethrin, bifenthrin, bioallethrin, bioethanomethrin, biopermethrin, bistrifluron, borax, boric acid, bromfenvinfos, bromocyclen, bromo-DDT, bromophos, bromophos-ethyl, bufencarb, buprofezin, butacarb, butathiofos, butocarboxim, butonate, butoxycarboxim, cadusafos, calcium arsenate, calcium polysulfide, camphechlor, carbanolate, carbaryl, carbofuran, carbon disulfide, carbon tetrachloride, carbophenothion, carbosulfan, cartap, cartap hydrochloride, chlorantraniliprole, chlorbicyclen, chlordane, chlordecone, chlordimeform, chlordimeform hydrochloride, chlorethoxyfos, chlorfenapyr, chlorfenvinphos, chlorfluazuron, chlormephos, chloroform, chloropicrin, chlorphoxim, chlorprazophos, chlorpyrifos, chlorpyrifos-methyl, chlorthiophos, chromafenozide, cinerin I, cinerin II, cinerins, cismethrin, cloethocarb, closantel, clothianidin, copper acetoarsenite, copper arsenate, copper naphthenate, copper oleate, coumaphos, coumithoate, crotamiton, crotoxyphos, crufomate, cryolite, cyanofenphos, cyanophos, cyanthoate, cyantraniliprole, cyclethrin, cycloprothrin, cyfluthrin, cyhalothrin, cypermethrin, cyphenothrin, cyromazine, cythioate, DDT, decarbofuran, deltamethrin, demephion, demephion-O, demephion-S, demeton, demeton-methyl, demeton-O, demeton-O-methyl, demeton-S, demeton-S-methyl, demeton-S-methylsulphon, diafenthiuron, dialifos, diatomaceous earth, diazinon, dicapthon, dichlofenthion, dichlorvos, dicresyl, dicrotophos, dicyclanil, dieldrin, diflubenzuron, dilor, dimefluthrin, dimefox, dimetan, dimethoate, dimethrin, dimethylvinphos, dimetilan, dinex, dinex-diclexine, dinoprop, dinosam, dinotefuran, diofenolan, dioxabenzofos, dioxacarb, dioxathion, disulfoton, dithicrofos, d-limonene, DNOC, DNOC-ammonium, DNOC-potassium, DNOC-sodium, doramectin, ecdysterone, emamectin, emamectin benzoate, EMPC, empenthrin, endosulfan, endothion, endrin, EPN, epofenonane, eprinomectin, esdepallethrine, esfenvalerate, etaphos, ethiofencarb, ethion, ethiprole, ethoate-methyl, ethoprophos, ethyl formate, ethyl-DDD, ethylene dibromide, ethylene dichloride, ethylene oxide, etofenprox, etrimfos, EXD, famphur, fenamiphos, fenazaflor, fenchlorphos, fenethacarb, fenfluthrin, fenitrothion, fenobucarb, fenoxacrim, fenoxycarb, fenpirithrin, fenpropathrin, fensulfothion, fenthion, fenthion-ethyl, fenvalerate, fipronil, flonicamid, flubendiamide, flucofuron, flucycloxuron, flucythrinate, flufenerim, flufenoxuron, flufenprox, fluvalinate, fonofos, formetanate, formetanate hydrochloride, formothion, formparanate, formparanate hydrochloride, fosmethilan, fospirate, fosthietan, furathiocarb, furethrin, gamma-cyhalothrin, gamma-HCH, halfenprox, halofenozide, HCH, HEOD, heptachlor, heptenophos, heterophos, hexaflumuron, HHDN, hydramethylnon, hydrogen cyanide, hydroprene, hyquincarb, imidacloprid, imiprothrin, indoxacarb, iodomethane, IPSP, isazofos, isobenzan, isocarbophos, isodrin, isofenphos, isofenphos-methyl, isoprocarb, isoprothiolane, isothioate, isoxathion, ivermectin, jasmolin I, jasmolin II, jodfenphos, juvenile hormone I, juvenile hormone II, juvenile hormone III, kelevan, kinoprene, lambda-cyhalothrin, lead arsenate, lepimectin, leptophos, lindane, lirimfos, lufenuron, lythidathion, malathion, malonoben, mazidox, mecarbam, mecarphon, menazon, mephosfolan, mercurous chloride, mesulfenfos, metaflumizone, methacrifos, methamidophos, methidathion, methiocarb, methocrotophos, methomyl, methoprene, methoxychlor, methoxyfenozide, methyl bromide, methyl isothiocyanate, methylchloroform, methylene chloride, metofluthrin, metolcarb, metoxadiazone, mevinphos, mexacarbate, milbemectin, milbemycin oxime, mipafox, mirex, molosultap, monocrotophos, monomehypo, monosultap, morphothion, moxidectin, naftalofos, naled, naphthalene, nicotine, nifluridide, nitenpyram, nithiazine, nitrilacarb, novaluron, noviflumuron, omethoate, oxamyl, oxydemeton-methyl, oxydeprofos, oxydisulfoton, para-dichlorobenzene, parathion, parathion-methyl, penfluron, pentachlorophenol, permethrin, phenkapton, phenothrin, phenthoate, phorate, phosalone, phosfolan, phosmet, phosnichlor, phosphamidon, phosphine, phoxim, phoxim-methyl, pirimetaphos, pirimicarb, pirimiphos-ethyl, pirimiphos-methyl, potassium arsenite, potassium thiocyanate, pp′-DDT, prallethrin, precocene I, precocene II, precocene III, primidophos, profenofos, profluralin, promacyl, promecarb, propaphos, propetamphos, propoxur, prothidathion, prothiofos, prothoate, protrifenbute, pyraclofos, pyrafluprole, pyrazophos, pyresmethrin, pyrethrin I, pyrethrin II, pyrethrins, pyridaben, pyridalyl, pyridaphenthion, pyrifluquinazon, pyrimidifen, pyrimitate, pyriprole, pyriproxyfen, quassia, quinalphos, quinalphos-methyl, quinothion, rafoxanide, resmethrin, rotenone, ryania, sabadilla, schradan, selamectin, silafluofen, silica gel, sodium arsenite, sodium fluoride, sodium hexafluorosilicate, sodium thiocyanate, sophamide, spinetoram, spinosad, spiromesifen, spirotetramat, sulcofuron, sulcofuron-sodium, sulfluramid, sulfotep, sulfoxaflor, sulfuryl fluoride, sulprofos, tau-fluvalinate, tazimcarb, TDE, tebufenozide, tebufenpyrad, tebupirimfos, teflubenzuron, tefluthrin, temephos, TEPP, terallethrin, terbufos, tetrachloroethane, tetrachlorvinphos, tetramethrin, tetramethylfluthrin, theta-cypermethrin, thiacloprid, thiamethoxam, thicrofos, thiocarboxime, thiocyclam, thiocyclam oxalate, thiodicarb, thiofanox, thiometon, thiosultap, thiosultap-disodium, thiosultap-monosodium, thuringiensin, tolfenpyrad, tralomethrin, transfluthrin, transpermethrin, triarathene, triazamate, triazophos, trichlorfon, trichlormetaphos-3, trichloronat, trifenofos, triflumuron, trimethacarb, triprene, vamidothion, vaniliprole, XMC, xylylcarb, zeta-cypermethrin, zolaprofos, and any combinations thereof.

Additionally, the compounds described herein may be combined with herbicides that are compatible with the compounds of the present disclosure in the medium selected for application, and not antagonistic to the activity of the present compounds to form pesticidal mixtures and synergistic mixtures thereof. The fungicidal compounds of the present disclosure may be applied in conjunction with one or more herbicides to control a wide variety of undesirable plants. When used in conjunction with herbicides, the presently claimed compounds may be formulated with the herbicide(s), tank-mixed with the herbicide(s) or applied sequentially with the herbicide(s). Typical herbicides include, but are not limited to: 4-CPA; 4-CPB; 4-CPP; 2,4-D; 3,4-DA; 2,4-DB; 3,4-DB; 2,4-DEB; 2,4-DEP; 3,4-DP; 2,3,6-TBA; 2,4,5-T; 2,4,5-TB; acetochlor, acifluorfen, aclonifen, acrolein, alachlor, allidochlor, alloxydim, allyl alcohol, alorac, ametridione, ametryn, amibuzin, amicarbazone, amidosulfuron, aminocyclopyrachlor, aminopyralid, amiprofos-methyl, amitrole, ammonium sulfamate, anilofos, anisuron, asulam, atraton, atrazine, azafenidin, azimsulfuron, aziprotryne, barban, BCPC, beflubutamid, benazolin, bencarbazone, benfluralin, benfuresate, bensulfuron, bensulide, bentazone, benzadox, benzfendizone, benzipram, benzobicyclon, benzofenap, benzofluor, benzoylprop, benzthiazuron, bicyclopyrone, bifenox, bilanafos, bispyribac, borax, bromacil, bromobonil, bromobutide, bromofenoxim, bromoxynil, brompyrazon, butachlor, butafenacil, butamifos, butenachlor, buthidazole, buthiuron, butralin, butroxydim, buturon, butylate, cacodylic acid, cafenstrole, calcium chlorate, calcium cyanamide, cambendichlor, carbasulam, carbetamide, carboxazole chlorprocarb, carfentrazone, CDEA, CEPC, chlomethoxyfen, chloramben, chloranocryl, chlorazifop, chlorazine, chlorbromuron, chlorbufam, chloreturon, chlorfenac, chlorfenprop, chlorflurazole, chlorflurenol, chloridazon, chlorimuron, chlornitrofen, chloropon, chlorotoluron, chloroxuron, chloroxynil, chlorpropham, chlorsulfuron, chlorthal, chlorthiamid, cinidon-ethyl, cinmethylin, cinosulfuron, cisanilide, clethodim, cliodinate, clodinafop, clofop, clomazone, clomeprop, cloprop, cloproxydim, clopyralid, cloransulam, CMA, copper sulfate, CPMF, CPPC, credazine, cresol, cumyluron, cyanatryn, cyanazine, cycloate, cyclosulfamuron, cycloxydim, cycluron, cyhalofop, cyperquat, cyprazine, cyprazole, cypromid, daimuron, dalapon, dazomet, delachlor, desmedipham, desmetryn, di-allate, dicamba, dichlobenil, dichloralurea, dichlormate, dichlorprop, dichlorprop-P, diclofop, diclosulam, diethamquat, diethatyl, difenopenten, difenoxuron, difenzoquat, diflufenican, diflufenzopyr, dimefuron, dimepiperate, dimethachlor, dimethametryn, dimethenamid, dimethenamid-P, dimexano, dimidazon, dinitramine, dinofenate, dinoprop, dinosam, dinoseb, dinoterb, diphenamid, dipropetryn, diquat, disul, dithiopyr, diuron, DMPA, DNOC, DSMA, EBEP, eglinazine, endothal, epronaz, EPTC, erbon, esprocarb, ethalfluralin, ethametsulfuron, ethidimuron, ethiolate, ethofumesate, ethoxyfen, ethoxysulfuron, etinofen, etnipromid, etobenzanid, EXD, fenasulam, fenoprop, fenoxaprop, fenoxaprop-P, fenoxasulfone, fenteracol, fenthiaprop, fentrazamide, fenuron, ferrous sulfate, flamprop, flamprop-M, flazasulfuron, florasulam, fluazifop, fluazifop-P, fluazolate, flucarbazone, flucetosulfuron, fluchloralin, flufenacet, flufenican, flufenpyr, flumetsulam, flumezin, flumiclorac, flumioxazin, flumipropyn, fluometuron, fluorodifen, fluoroglycofen, fluoromidine, fluoronitrofen, fluothiuron, flupoxam, flupropacil, flupropanate, flupyrsulfuron, fluridone, flurochloridone, fluroxypyr, flurtamone, fluthiacet, fomesafen, foramsulfuron, fosamine, furyloxyfen, glufosinate, glufosinate-P, glyphosate, halauxifen, halosafen, halosulfuron, haloxydine, haloxyfop, haloxyfop-P, hexachloroacetone, hexaflurate, hexazinone, imazamethabenz, imazamox, imazapic, imazapyr, imazaquin, imazethapyr, imazosulfuron, indanofan, indaziflam, iodobonil, iodomethane, iodosulfuron, ioxynil, ipazine, ipfencarbazone, iprymidam, isocarbamid, isocil, isomethiozin, isonoruron, isopolinate, isopropalin, isoproturon, isouron, isoxaben, isoxachlortole, isoxaflutole, isoxapyrifop, karbutilate, ketospiradox, lactofen, lenacil, linuron, MAA, MAMA, MCPA, MCPA-thioethyl, MCPB, mecoprop, mecoprop-P, medinoterb, mefenacet, mefluidide, mesoprazine, mesosulfuron, mesotrione, metam, metamifop, metamitron, metazachlor, metazosulfuron, metflurazon, methabenzthiazuron, methalpropalin, methazole, methiobencarb, methiozolin, methiuron, methometon, methoprotryne, methyl bromide, methyl isothiocyanate, methyldymron, metobenzuron, metobromuron, metolachlor, metosulam, metoxuron, metribuzin, metsulfuron, molinate, monalide, monisouron, monochloroacetic acid, monolinuron, monuron, morfamquat, MSMA, naproanilide, napropamide, naptalam, neburon, nicosulfuron, nipyraclofen, nitralin, nitrofen, nitrofluorfen, norflurazon, noruron, OCH, orbencarb, ortho-dichlorobenzene, orthosulfamuron, oryzalin, oxadiargyl, oxadiazon, oxapyrazon, oxasulfuron, oxaziclomefone, oxyfluorfen, parafluron, paraquat, pebulate, pelargonic acid, pendimethalin, penoxsulam, pentachlorophenol, pentanochlor, pentoxazone, perfluidone, pethoxamid, phenisopham, phenmedipham, phenmedipham-ethyl, phenobenzuron, phenylmercury acetate, picloram, picolinafen, pinoxaden, piperophos, potassium arsenite, potassium azide, potassium cyanate, pretilachlor, primisulfuron, procyazine, prodiamine, profluazol, profluralin, profoxydim, proglinazine, prometon, prometryn, propachlor, propanil, propaquizafop, propazine, propham, propisochlor, propoxycarbazone, propyrisulfuron, propyzamide, prosulfalin, prosulfocarb, prosulfuron, proxan, prynachlor, pydanon, pyraclonil, pyraflufen, pyrasulfotole, pyrazolynate, pyrazosulfuron, pyrazoxyfen, pyribenzoxim, pyributicarb, pyriclor, pyridafol, pyridate, pyriftalid, pyriminobac, pyrimisulfan, pyrithiobac, pyroxasulfone, pyroxsulam, quinclorac, quinmerac, quinoclamine, quinonamid, quizalofop, quizalofop-P, rhodethanil, rimsulfuron, saflufenacil, S-metolachlor, sebuthylazine, secbumeton, sethoxydim, siduron, simazine, simeton, simetryn, SMA, sodium arsenite, sodium azide, sodium chlorate, sulcotrione, sulfallate, sulfentrazone, sulfometuron, sulfosulfuron, sulfuric acid, sulglycapin, swep, TCA, tebutam, tebuthiuron, tefuryltrione, tembotrione, tepraloxydim, terbacil, terbucarb, terbuchlor, terbumeton, terbuthylazine, terbutryn, tetrafluron, thenylchlor, thiazafluron, thiazopyr, thidiazimin, thidiazuron, thiencarbazone-methyl, thifensulfuron, thiobencarb, tiocarbazil, tioclorim, topramezone, tralkoxydim, triafamone, tri-allate, triasulfuron, triaziflam, tribenuron, tricamba, triclopyr, tridiphane, trietazine, trifloxysulfuron, trifluralin, triflusulfuron, trifop, trifopsime, trihydroxytriazine, trimeturon, tripropindan, tritac, tritosulfuron, vernolate, and xylachlor.

Another embodiment of the present disclosure is a method for the control or prevention of fungal attack. This method comprises applying to the soil, plant, roots, foliage, or locus of the fungus, or to a locus in which the infestation is to be prevented (for example applying to cereal or grape plants), a fungicidally effective amount of one or more of the compounds of Formula I. The compounds are suitable for treatment of various plants at fungicidal levels, while exhibiting low phytotoxicity. The compounds may be useful both in a protectant and/or an eradicant fashion.

The compounds have been found to have significant fungicidal effect particularly for agricultural use. Many of the compounds are particularly effective for use with agricultural crops and horticultural plants.

It will be understood by those skilled in the art that the efficacy of the compound for the foregoing fungi establishes the general utility of the compounds as fungicides.

The compounds have broad ranges of activity against fungal pathogens. Exemplary pathogens may include, but are not limited to, causing agent of wheat leaf blotch (Zymoseptoria tritici), wheat brown rust (Puccinia triticina), wheat stripe rust (Puccinia striiformis), scab of apple (Venturia inaequalis), powdery mildew of grapevine (Uncinula necator), barley scald (Rhynchosporium secalis), blast of rice (Magnaporthe grisea), rust of soybean (Phakopsora pachyrhizi), glume blotch of wheat (Leptosphaeria nodorum), powdery mildew of wheat (Blumeria gramninis f sp. tritici), powdery mildew of barley (Blumeria gramninis f sp. hordei), powdery mildew of cucurbits (Erysiphe cichoracearum), anthracnose of cucurbits (Glomerella lagenarium), leaf spot of beet (Cercospora beticola), early blight of tomato (Alternaria solani), and spot blotch of barley (Cochliobolus sativus). The exact amount of the active material to be applied is dependent not only on the specific active material being applied, but also on the particular action desired, the fungal species to be controlled, and the stage of growth thereof, as well as the part of the plant or other product to be contacted with the compound. Thus, all the compounds, and formulations containing the same, may not be equally effective at similar concentrations or against the same fungal species.

The compounds are effective in use with plants in a disease-inhibiting and phytologically acceptable amount. The term “disease-inhibiting and phytologically acceptable amount” refers to an amount of a compound that kills or inhibits the plant disease for which control is desired, but is not significantly toxic to the plant. This amount will generally be from about 0.1 to about 1000 ppm (parts per million), with 1 to 500 ppm being preferred. The exact concentration of compound required varies with the fungal disease to be controlled, the type of formulation employed, the method of application, the particular plant species, climate conditions, and the like. A suitable application rate is typically in the range from about 0.10 to about 4 pounds/acre (about 0.01 to 0.45 grams per square meter, g/m²).

Any range or desired value given herein may be extended or altered without losing the effects sought, as is apparent to the skilled person for an understanding of the teachings herein.

The compounds of Formula I may be made using well-known chemical procedures. Intermediates not specifically mentioned in this disclosure are either commercially available, may be made by routes disclosed in the chemical literature, or may be readily synthesized from commercial starting materials utilizing standard procedures.

### General Schemes

The following schemes illustrate approaches to generating picolinamide compounds of Formula (I). The following descriptions and examples are provided for illustrative purposes and should not be construed as limiting in terms of substituents or substitution patterns. Additionally, the substitutions about Formula I as described in the Markush structure can often be identified early in the synthetic schemes. However, one skilled in the art will recognize that many of these substitutions are simply a part of the protection/deprotection strategy used to synthesize these compounds. As such, only the substitutions about the macrocycle, i.e., post cyclization, and those that are relevant to the final targets will be described in terms of the Markush structure.

The macrocycle of Formula 1.8, wherein X and Y are tert-butoxycarbonyl, R₁ and R₂ are OR₃, R₃ is hydrogen, Z₁ is oxygen, and Z₂ is methylene (—CH₂), can be prepared according to the method outlined in Scheme 1, Steps a-g. The bis(benzyloxy) substituted 3,4-dihydropyran of Formula 1.1 can be prepared from the corresponding bis(acetoxy) substituted 3,4-dihydropyran of Formula 1.0 by treating a solution of the acetoxy starting material (SM) in a polar, protic solvent like methanol (MeOH) with an alkali carbonate base, such as potassium carbonate (K₂CO₃), at an ambient temperature of about 21° C. to give the intermediate dihydroxy substituted 3,4-dihydropyran. Treating a solution of the dihydroxy intermediate in a polar solvent like N,N-dimethylformamide (DMF) at a reduced temperature of about 0° C. with a strong base, for example sodium hydride (NaH), affords the dianion which may be reacted with an electrophile, such as benzyl bromide (BnBr), at a temperature from about 0° C. to about 21° C. to give the bis(benzyloxy) compound of Formula 1.1, as shown in a. The acyclic diol of Formula 1.2 can be prepared via an oxymercuration-reduction sequence by treating the 3,4-dihdropyran of Formula 1.1 with mercuric acetate (Hg(OAc)₂) in aqueous (aq) tetrahydrofuran (THF) to give the acetoxymercury adduct, which undergoes reductive elimination by treating with sodium borohydride (NaBH₄) at a reduced temperature of about 0° C., as depicted in b. The secondary (2°) alcohol of Formula 1.4 can be prepared by treating a solution of the acyclic diol of Formula 1.2 in the presence of a Lewis acid, for example boron trifluoride-diethyl etherate (BF₃ OEt₂), with a protected aziridine, for example the tert-butyl carbamate (Boc) protected aziridine of Formula 1.3, in a halogenated organic solvent like dichloromethane (CH₂Cl₂), at a reduced temperature of about −78° C. to about 0° C., as shown in c. The seco acid of Formula 1.5 can be obtained by subjecting the methyl ester of Formula 1.4 to standard saponification conditions, for example by treating a solution of the ester in a mixture of water (H₂O) and a polar organic solvent, such as THF or MeOH, with a hydroxide base, for example lithium hydroxide (LiOH), at a temperature of about 21° C., as depicted in d. The compound of Formula 1.6, wherein X is hydrogen, Y is tert-butoxycarbonyl, R₁ and R₂ are OR₃, R₃ is benzyl, Z₁ is oxygen, and Z₂ is methylene (—CH₂), can be prepared by adding a solution of the seco acid of Formula 1.5 in a halogenated solvent like CH₂Cl₂ or an aromatic solvent like toluene to a mixture of a base, such as N,N-dimethylaminopyridine (DMAP), and an anhydride, such as 2-methyl-6-nitrobenzoic anhydride (MNBA), in either a halogenated solvent like CH₂Cl₂ or an aromatic solvent like toluene over a period of 4-12 hours (h), at a temperature between about 21° C. and about 70° C., as shown in e. The compound of Formula 1.7, wherein X and Y are tert-butoxycarbonyl and R₁, R₂, R₃, Z₁ and Z₂ are as defined above, can be prepared by treating a solution of the mono-Boc compound of Formula 1.6 in a polar, aprotic solvent like acetonitrile (CH₃CN) with di-tert-butyl dicarbonate (Boc₂O) in the presence of DMAP at a temperature of about 21° C., as shown in f The macrocycle of Formula 1.8, wherein R₁, R₂, R₃, X, Y, Z₁, and Z₂ are as previously defined, can be prepared by treating the compound of Formula 1.7 in a polar, aprotic solvent like THF with a metal catalyst, such as palladium on carbon (Pd/C), in the presence of hydrogen gas (H₂) at a pressure of about 600 pounds per square inch (psi) and an elevated temperature of about 40° C., as shown in g.

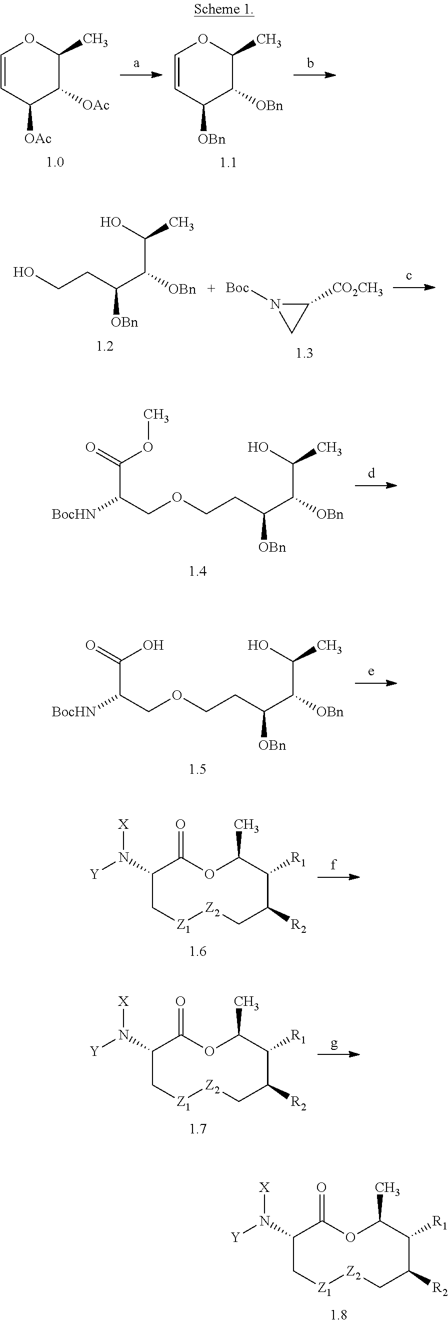

Macrocycles of Formula 2.3, wherein X and Y are tert-butoxycarbonyl, R₁ is OR₃, R₂ is as originally defined, R₃ is hydrogen, Z₁ is oxygen, and Z₂ is methylene, can be prepared according to the method outlined in Scheme 2, Steps a-d. The 3-benzyloxy substituted 3,4-dihydropyran of Formula 2.0 can be prepared from the corresponding bis(acetoxy) substituted 3,4-dihydropyran of Formula 1.0 by reacting a mixture of the acetoxy SM, a phase transfer catalyst, for example tetrabutylammonium iodide (NBu₄I), an alkali hydroxide base, for example sodium hydroxide (NaOH), and an electrophile, for example BnBr, in a mixture of H₂O and a non-miscible organic solvent, for example CH₂Cl₂ or, in some cases in which the electrophile is a liquid, such as BnBr, the electrophile may serve as the organic solvent. Stirring the above mixture at about 21° C. for a period of 4-7 days (d) affords the dihydropyran of Formula 2.0, as shown in a. The 4-hydroxy substituted 3,4 dihydropyran of Formula 2.1 can be prepared treating a solution of the benzyloxy compound of Formula 2.0 in a polar, protic solvent like MeOH with an alkali carbonate base, such as K₂CO₃, at an ambient temperature of about 21° C., as shown in b. Substituted 3,4-dihydropyrans of Formula 2.2, wherein R₂ is as originally defined, for example OR₃, wherein R₃ is alkyl, can be prepared by treating the alcohol of Formula 2.1 with a strong base, for example NaH, and an electrophile, for example an alkylating agent like an alkyl halide or sulfonate in an anhydrous, polar solvent like DMF at a reduced temperature of about 0° C., as shown in c. The macrocycles of Formula 2.3, wherein X, Y, R₁, R₂, R₃, Z₁, and Z₂ are as previously defined, can be prepared from compounds of Formula 2.2, wherein R₂ is as previously defined, according to the 6-step procedure outlined in Scheme 1, Steps b-g, as shown in d.

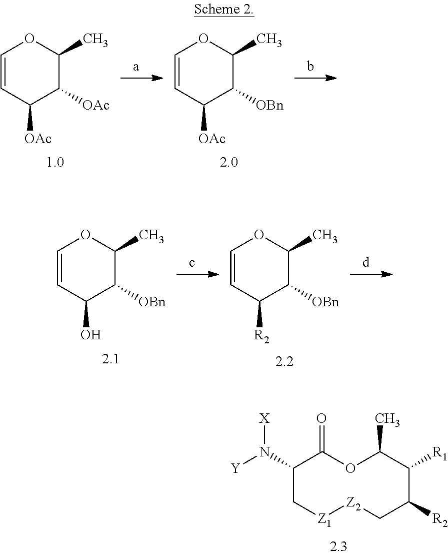

Compounds of Formulae 3.3-3.7, wherein R₁ is OR₃ and R₂ and R₃ are as originally defined, can be prepared by the method shown in Scheme 3, Steps a-f Compounds of Formula 3.1, wherein R₃ is as originally defined and R₂₀ is alkyl or alkoxy, can be prepared from compounds of Formula 3.0, wherein R₃ is as originally defined, by treatment with an alkoxy borane, such as pinacol borane, in the presence of a nickel catalyst, such as bis(cyclooctadiene)nickel(0) (Ni(cod)₂), as described by Ely, R. J.; Morken, J. P. J. Am. Chem. Soc. 2010, 132, 2534-2535, in a solvent such as toluene at a temperature between about 0° C. and 23° C. Alternatively, compounds of Formula 3.1, wherein R₃ is as originally defined and R₂₀ is alkyl or alkoxy, can be prepared as reported in Brown, H. C.; Bhat, K. S.; Randad, R. S. J. Org. Chem. 1989, 54, 1570. Compounds of Formula 3.3, wherein R₁ is OR₃, R₂ is as originally defined, and R₃ is hydrogen, can be prepared from compounds of Formula 3.1, wherein R₃ is as originally defined and R₂ is as defined above, by treatment with a benzyl (Bn) orpara-methoxybenzyl (PMB) protected, lactate-derived aldehyde such as compound 3.2, prepared as described in Cheng and Brookhart Angew. Chem. Int. Ed. 2012, 51, 9422-9424 (see Takai, K.; Heathcock, C. H. J. Org. Chem. 1985, 50, 3247-3251 for characterization of Bn aldehyde and Terashima et al. Bull. Chem. Soc. Jpn. 1989, 62, 3038-3040 for characterization of PMB aldehyde), as shown in b. Compounds of Formula 3.4, wherein R₁ is OR₃, R₂ is as defined above, and R₃ is acyl, can be prepared by treating the compound of Formula 3.3, wherein R₁ is OR₃, R₂ is as defined above, and R₃ is hydrogen, with an organic amine base, such as DMAP, triethylamine (NEt₃), or mixtures thereof, followed by an acyl halide at a temperature of about 21° C., as shown in c. Compounds of Formula 3.5, wherein R₁ is OR₃, R₂ is as defined above, and R₃ is aryl, can be prepared by treating a compound of Formula 3.3, wherein R₁ is OR₃, R₂ is as defined above, and R₃ is hydrogen, with a triarylbismuth reagent, prepared according to the methods described by Hassan, A. et. al. Organometallics 1996, 15, 5613-5621, Moiseev, D. V. et al. J. Organomet. Chem. 2005, 690, 3652-3663, or Sinclair, P. J. et al. Bioorg. Med. Chem. Lett. 1995, 5, 1035-1038, in the presence of a copper catalyst, such as diacetoxycopper, and an amine base, such as N,N-dicyclohexyl-methylamine, in an aprotic solvent like toluene at a temperature between about 21° C. and about 50° C., as shown in d. Compounds of Formula 3.6, wherein R₁ is OR₃, R₂ is as defined above, and R₃ is alkyl, can be prepared by treating a compound of Formula 3.3, wherein R₁ is OR₃, R₂ is as defined above, and R₃ is hydrogen, with a strong base, such as potassium tert-butoxide (KO^{t}Bu) or NaH, in a polar, aprotic solvent like THF, followed by treatment of the resultant anion with an alkyl halide or sulfonate, at a temperature from about 21° C. to about 40° C., as shown in e. Compounds of Formula 3.7, wherein R₁ is OR₃, R₂ is as defined above, and R₃ is silyl, can be prepared by treating a compound of Formula 3.3, wherein R₁ is OR₃, R₂ is as defined above, and R₃ is hydrogen, with an organic amine base like 2,6-lutidine, in an aprotic solvent like CH₂Cl₂ with a silylating reagent, for example triisopropylsilyl trifluoromethanesulfonate (TIPS-OTf) at a reduced temperature of about 0° C. to about 21° C., as shown in f.

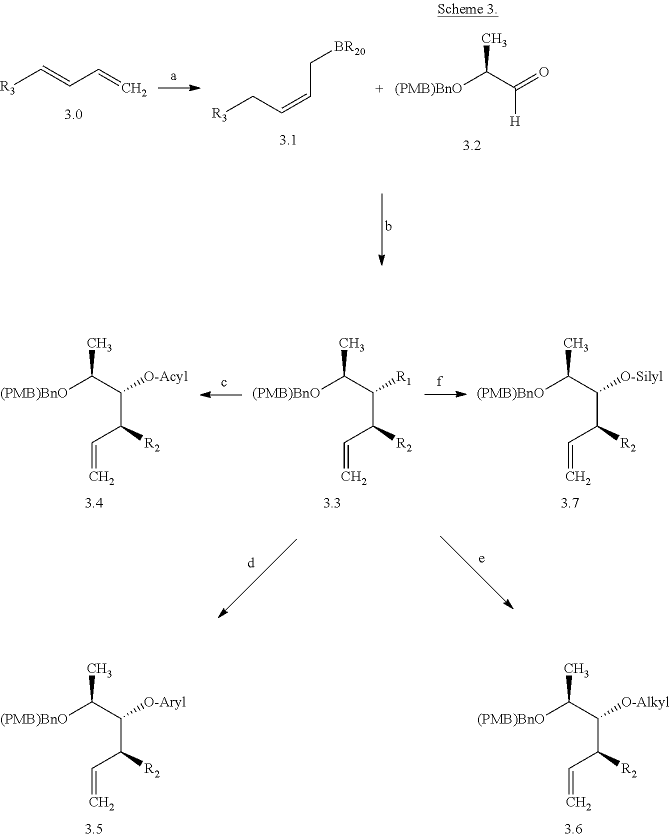

Macrocycles of Formula 4.6, wherein X is hydrogen, Y is tert-butoxycarbonyl, R₁ and R₂ are as originally defined, but not alkenyl or benzyl, Z₁ is oxygen, and Z₂ is methylene, can be prepared according to the method outlined in Scheme 4, Steps a-f Alcohols of Formula 4.1, wherein R₁ and R₂ are as defined above, can be prepared by reacting compounds of Formula 4.0, wherein R₁ and R₂ are as defined above, under standard hydroboration conditions, i.e., treating the compound of Formula 4.0 with a source of borane, for example borane THF complex (BH₃-THF), at a temperature of about 21° C. Following the hydroboration, oxidation of the intermediate boron species can be achieved by reacting with the conjugate base of hydrogen peroxide (NaO—OH), generated by deprotonating hydrogen peroxide (H₂O₂) with an alkali hydroxide base, for example NaOH, at a reduced temperature of about 0° C., as shown in a. Esters of Formula 4.3, wherein R₁ and R₂ are as defined above, can be prepared as shown in b by reacting compounds of Formula 4.1, wherein R₁ and R₂ are as previously defined, with either the methyl (Me) or Bn ester of the Boc protected aziridine of Formula 4.2, using the methodology described in Scheme 1, Step c. The secondary alcohols of Formula 4.4, wherein R₁ and R₂ are as defined above, can be prepared from the Me ester of Formula 4.3, wherein R₁ and R₂ are as previously defined, by reacting with H₂ in the presence of a metal catalyst, for example Pd/C (Degussa), in an aprotic solvent like ethyl acetate (EtOAc) at a temperature of about 21° C. and a pressure of about 1 atmosphere (Atm), as shown in c. The seco acid of Formula 4.5, wherein R₁ and R₂ are as defined above, can be prepared by subjecting the ester of Formula 4.4, wherein R₁ and R₂ are as previously defined, to the standard saponification conditions described in Scheme 1, Step d, as shown in d. Alternatively, the seco acid of Formula 4.5, wherein R₁ and R₂ are as defined above, can be prepared from the Bn ester of Formula 4.3, wherein R₁ and R₂ are as previously defined, using the hydrogenolysis conditions described in Step c, as shown in e. The macrocycles of Formula 4.6, wherein X, Y, R₁, R₂, Z₁, and Z₂ are as defined above, can be prepared by the addition a solution of the seco acid of Formula 4.5, wherein R₁ and R₂ are as previously defined, in a halogenated solvent such as CH₂Cl₂ or an aromatic solvent such as toluene to a mixture of a base, such as DMAP, and an anhydride, such as MNBA, in either a halogenated solvent such as CH₂Cl₂ or an aromatic solvent such as toluene over a period of 4-12 h, at a temperature between about 21° C. and about 70° C., as described in Scheme 1, Step e and shown in f.

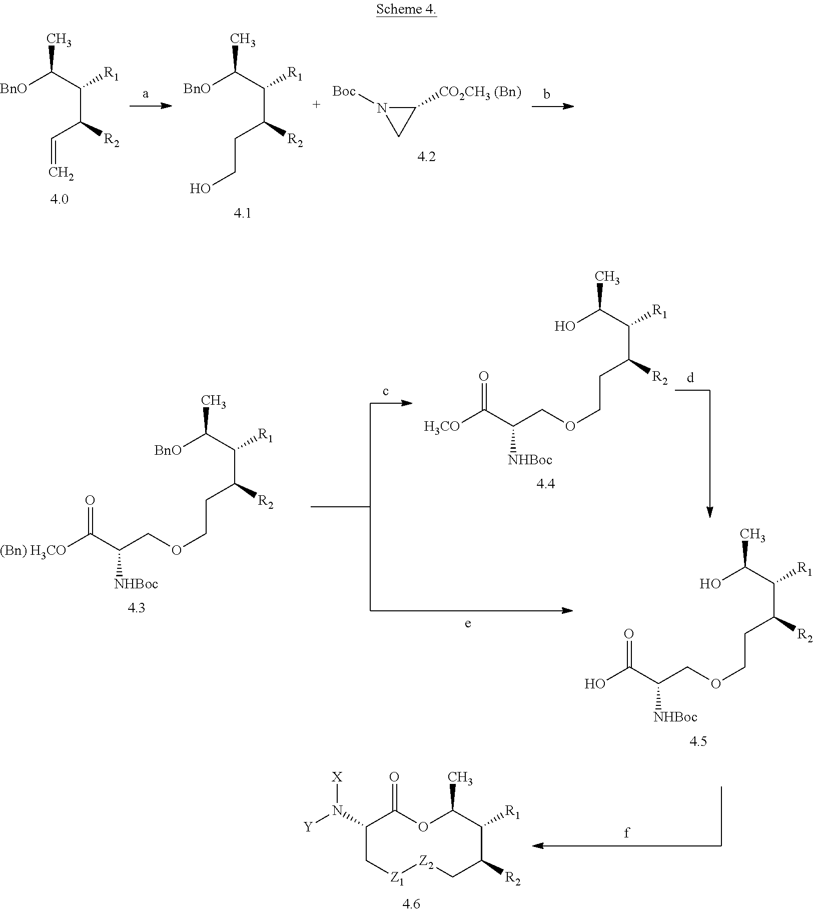

Macrocycles of Formula 5.7, wherein X is hydrogen, Y is tert-butoxycarbonyl, R₁ is OR₃, R₂ is as originally defined, but not alkenyl, R₃ is hydrogen, and Z₁ and Z₂ are methylene, can be prepared according to the method outlined in Scheme 5, Steps a-g. The aldehydes of Formula 5.0, wherein R₁ is OR₃, R₂ is as defined above, and R₃ is silyl, can be prepared by subjecting compounds of Formula 3.7, wherein R₁ and R₂ are as defined above, to oxidative conditions, for example treatment with sulfur trioxide pyridine complex (SO₃ pyridine) in the presence of an organic amine base, such as NEt₃, in a mixed solvent system, for example about 20% dimethylsulfoxide (DMSO) in CH₂Cl₂, at a reduced temperature of about 0° C., as shown in a. Alkenes of Formula 5.1, wherein R₁ and R₂ are as defined above, can be prepared from aldehydes of Formula 5.0, wherein R₁ and R₂ are as previously defined, using standard Wittig olefination conditions. For example, adding aldehydes of Formula 5.0 to an ylide, such as the triphenylphosphonium methylide, generated by treating a solution of methyltriphenylphosphonium bromide in a polar, aprotic solvent like THF with a stong base, such as KO^{t}Bu, at a temperature of about 22° C., at a reduced temperature of about 0° C., as shown in b. Alkenes of Formula 5.1, wherein R₁ and R₂ are as previously defined, can be further functionalized through a hydroboration-Suzuki sequence in which the alkene is treated with an organoborane, such as 9-borabicyclo[3.3.1]nonane (9-BBN), in a polar, aprotic solvent like THF at a temperature of about 22° C. The resulting alkylborane may be treated with a solution of a vinyl halide, such as the bromoacrylate of Formula 5.2 in a polar solvent like DMF, in the presence of a base, such as potassium phosphate (K₃PO₄) or K₂CO₃, and a palladium catalyst, such as [1,1′-bis(diphenylphosphino)ferrocene]dichloropalladium(II), complex with CH₂Cl₂ (Pd(dppf)Cl₂.CH₂Cl₂), to give the cross-coupled alkene products of Formula 5.3, wherein R₁ and R₂ are as defined above, as shown in c. The alkenes of Formula 5.3, wherein R₁ and R₂ are as previously defined, may be subjected to assymetric hydrogenation conditions, for example treatment with chiral catalysts, such as (S,S)-Et-Rh-Duphos, in a polar solvent like MeOH in the presence of H₂ at a pressure of about 200 p.s.i. to give the reduced products of Formula 5.4, wherein R₁ and R₂ are as defined above, as shown in d. The seco acids of Formula 5.5, wherein R₁ and R₂ are as defined above, can be prepared from the Bn-protected precursors of Formula 5.4, wherein R₁ and R₂ are as previously defined, by treating with a metal catalyst, such as Pd/C, in a polar solvent like EtOAc and reacting with H₂ at a temperature of about 22° C. and a pressure of about 1 Atm, as shown in e. The compounds of Formula 5.6, wherein X, Y, R₂, Z₁, and Z₂ are as defined above, R₁ is OR₃, and R₃ is silyl, can be prepared from the seco acids of Formula 5.5, wherein R₁ and R₂ are as previously defined, using the methodology described in Scheme 1, Step e, and shown in f. The macrocycles of Formula 5.7, wherein X, Y, R₁, R₂, R₃, Z₁, and Z₂ are as previously defined, can be prepared from the compounds of Formula 5.6, wherein X, Y, R₁, R₂, R₃, Z₁, and Z₂ are as previously defined, by treating with a fluoride source, such as tetra-n-butylammonium fluoride (TBAF), in a polar, aprotic solvent like THF at about 22° C., as shown in g.

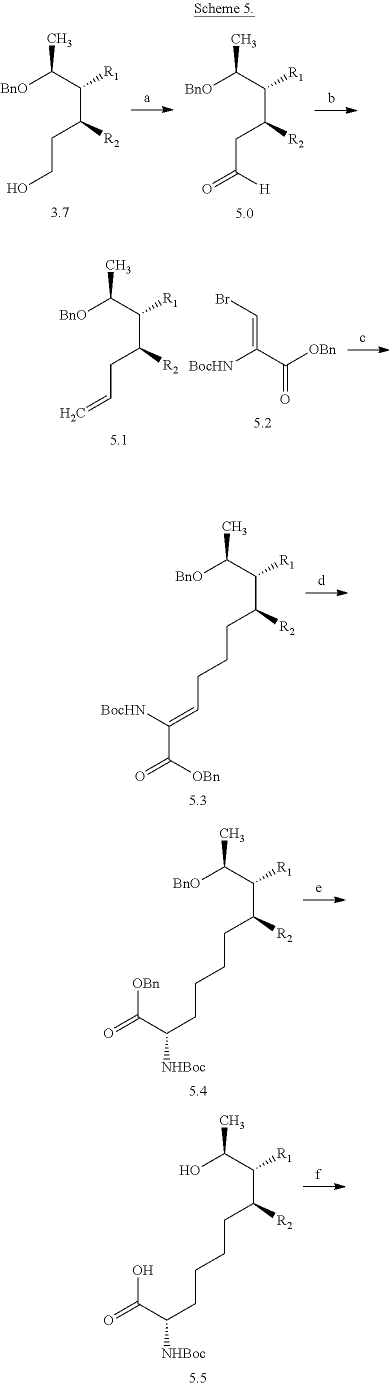

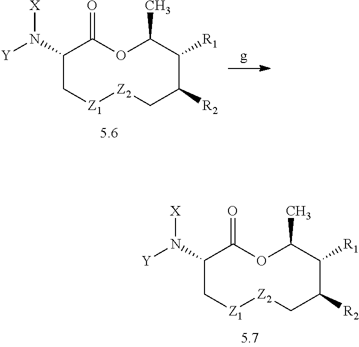

Macrocycles of Formula 6.10, wherein X is hydrogen, Y is tert-butoxycarbonyl, R₁ and R₂ are OR₃, R₃ is hydrogen, Z₁ is methylene and Z₂ is oxygen, can be prepared according to the method outlined in Scheme 6, Steps a-k. The dihydroxy substituted 3,4-dihydropyran of Formula 6.0 can be prepared from the corresponding bis(acetoxy) substituted 3,4-dihydropyran of Formula 1.0 by treating a solution of the acetoxy SM in a polar, protic solvent like MeOH with an alkali carbonate base, such as K₂CO₃, at an ambient temperature of about 22° C. to give the intermediate dihydroxy substituted 3,4-dihydropyran, as shown in a. The bis p-methoxybenzyl ether protected (OPMB) 3,4-dihydropyran of Formula 6.1 can be prepared by treating the compound of Formula 6.0 with a strong base, such as NaH, in a polar solvent like DMF and quenching the resultant dianion with 1-(bromomethyl)-4-methoxybenzene at a temperature from about 0° C. to about 22° C., as shown in b. It is noteworthy that the addition of a scavenger, such as diethylamine, to the completed reaction mixture at about 0° C. is required to consume the residual PMBBr and prevent the formation of the deleterious hydrogen bromide (HBr) that would form during work-up or purification. The tetrahydrofuran of Formula 6.2 can be prepared from 3,4-dihydropyrans of Formula 6.1 through ozonolysis with a reductive work-up, followed by saponification of the resultant formate ester and intramolecular cyclization between the newly formed aldehyde and alcohol moieties. For example, dihydropyrans of Formula 6.1 can be treated with ozone (O₃) in a solvent mixture such as CH₂Cl₂ and MeOH at a temperature of about −78° C., in the presence of a catalytic amount of an alkali carbonate base, such as sodium bicarbonate (NaHCO₃), and an indicator, such as 1-(4-(phenyldiazenyl)phenyl) azonaphthalen-2-ol (Sudan III), followed by the addition of dimethylsulfide ((CH₃)₂S) to give the intermediate, linear formate ester, which can be saponified and cyclized using the standard saponification conditions described in Scheme 1, Step d, as shown in c. The diol of Formula 6.3 can be prepared from the lactol of Formula 6.2 by treating with a hydride source, such as NaBH₄, in a polar, protic solvent like ethanol (EtOH) at a temperature of about 21° C., as shown in d. The alkenyl ether of Formula 6.4 can be prepared from the diol of Formula 6.3 by reacting with an alkyl halide, such as 1-bromo-3-methylbut-2-ene, in the presence of a base, such as about 3 molar (M) NaOH, and a phase transfer catalyst, such as N,N-dibutyl-N-methylbutan-1-aminium chloride, at about 21° C., as shown in e. The aldehyde of Formula 6.5 can be prepared from the alkenyl ether of Formula 6.4 using the ozonolysis conditions described is step c, as shown in f. The Boc-protected alkenyl ether of Formula 6.6 can be prepared from the aldehyde of Formula 6.5 using standard Homer-Wadsworth-Emmons conditions. For example, treating solutions of the aldehyde in an aprotic solvent like CH₂Cl₂ with a phosphonate, such as methyl 2-((tert-butoxycarbonyl)amino)-2-(dimethoxyphosphoryl)acetate, with a base, such as 1,8-diazabicyclo[5.4.0]undec-7-ene (DBU), at a temperature of about 21° C., as shown in g. The alkyl ether of Formula 6.7 can be prepared from the alkenyl ether of Formula 6.6 using the assymetric hydrogenation conditions described in Scheme 5, Step d, as shown in h. The seco acid of Formula 6.8 can be prepared from the methyl ester of Formula 6.7 using the saponification conditions described in Scheme 1, Step d, as shown in i. The macrocycle of Formula 6.9, wherein X is hydrogen, Y is tert-butoxycarbonyl, R₁ and R₂ are OR₃, R₃ is PMB, Z₁ is methylene and Z₂ is oxygen, can be prepared from the seco acid of Formula 6.8 using the conditions described in Scheme 1, Step e, as shown in j. The macrocycle of Formula 6.10, wherein X, Y, R₁, R₂, R₃, Z₁, and Z₂ are as previously defined, can be prepared from the macrocycle of Formula 6.9, wherein X, Y, R₁, R₂, R₃, Z₁, and Z₂ are as previously defined, by treating with an oxidant, such as ceric ammonium nitrate (CAN) or 2,3-dichloro-5,6-dicyano-p-benzoquinone (DDQ), in a mixed solvent system like about 10% H₂O in CH₃CN at about 0° C., as shown in k.

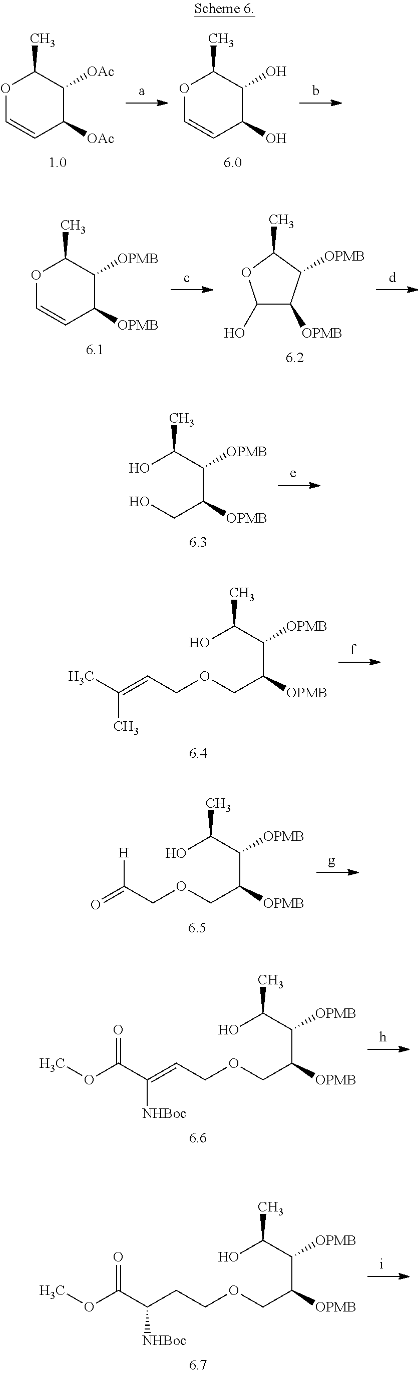

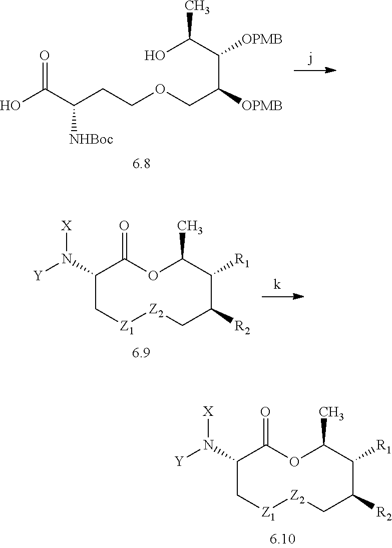

Macrocycles of Formula 7.4, wherein X and Y are tert-butoxycarbonyl, R₁ and R₂ are OR₃, R₃ is hydrogen, and Z₁ and Z₂ are methylene, can be prepared according to the method outlined in Scheme 7, Steps a-e. The tetrahydropyran of Formula 7.0 can be prepared via the hydration of the 2, 3-dihydropyran of Formula 1.1 catalyzed by a cation exchange resin, such as Dowex® 50WX4, in the presence of lithium bromide (LiBr) in aq CH₃CN, wherein the ratio of CH₃CN to H₂O is about 56:1 v/v, at a temperature of about 21° C., as shown in a. The alkenyl alcohol of Formula 7.1 can be prepared from the tetrahydropyran of Formula 7.0 using the Wittig olefination conditions described in Scheme 5, Step b, but employing n-butyllithium (n-BuLi) to generate the triphenylphosphonium methylide at about 0° C. and reacting the ylide with the tetrahydropyran at about −78° C., as shown in b. The acetate of Formula 7.2 can be prepared from the alcohol of Formula 7.1 by treating with an organic amine base, such as NEt₃, DMAP, or mixtures thereof, and acetic anhydride in a solvent like CH₂Cl₂ at a reduced temperature of about 0° C., as shown in c. The methyl ester of Formula 7.3 can be prepared from the acetate of Formula 7.2 using the hydroboration-Suzuki sequence described in Scheme 5, Step c, as shown in d. The macrocycle of Formula 7.4, wherein X, Y, R₁, R₂, R₃, Z₁ and Z₂ are as previously defined, can be prepared from the methyl ester of Formula 7.3, wherein X, Y, R₁, R₂, R₃, Z₁, and Z₂ are as previously defined, using the asymmetric hydrogenation conditions described in Scheme 5, Step d, the acetate cleavage conditions described in Scheme 6, Step a, and the saponification, lactonization, Boc protection, and hydrogenolysis conditions described in Scheme 1, Step d-g, as shown in e.

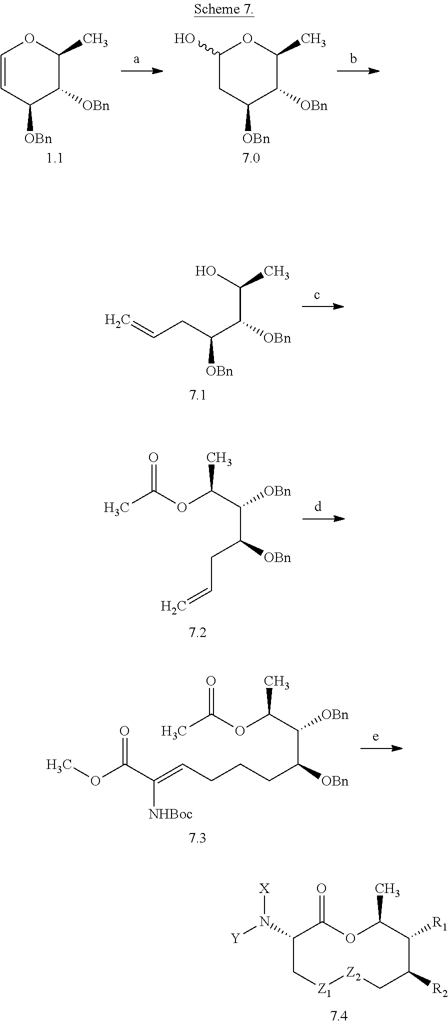

Macrocycles of Formulae 8.1-8.10, wherein X, Y, R₁, R₂, Z₁, and Z₂ are as originally defined, can be prepared according to the methods outlined in Scheme 8, Steps a-g. Compounds of Formula 8.0, wherein X is hydrogen, Y is tert-butoxycarbonyl, R₁ and R₂ are OR₃, R₃ is hydrogen, and Z₁ and Z₂ are methylene, can be subjected to the arylation conditions described in Scheme 3, Step d, to give the mixture of arylated compounds of Formulae 8.1-8.3, wherein X, Y, Z₁, and Z₂ are as previously defined and, where indicated, R₁ and R₂ are OR₃ and R₃ is hydrogen, as shown in a. Compounds of Formula 8.0, wherein X and Y are tert-butoxycarbonyl, R₁ and R₂ are OR₃, R₃ is hydrogen, and Z₁ and Z₂ are methylene, can be subjected to the acylation conditions described in Scheme 3, Step c, to give the mixture of acylated compounds of Formulae 8.4-8.5, wherein X, Y, Z₁, and Z₂ are as previously defined and, where indicated, R₁ and R₂ are OR₃ and R₃ is hydrogen, as shown in b. Compounds of Formula 8.0, wherein X and Y are tert-butoxycarbonyl, R₁ is OR₃, R₂ is as originally defined, R₃ is hydrogen, and Z₁ and Z₂ are methylene, may be treated with a symmetric or mixed carbonate, such as bis(2-methylallyl) carbonate or tert-butyl(2-methylallyl) carbonate, respectively, in the presence of a ligand, such as 1,1′-bis(diphenyl-phosphino)ferrocene (dppf), and a palladium catalyst, such as tris(dibenzylideneacetone)-dipalladium(0) (Pd₂(dba)₃), in a polar, aprotic solvent like THF at a temperature of about 60° C. to give compounds of Formula 8.6, wherein X, Y, R₂, Z₁ and Z₂ are as previously defined and R₁ is OR₃, wherein R₃ is alkenyl, such as an allylic moiety, as shown in c. Compounds of Formula 8.7, wherein X and Y are tert-butoxycarbonyl, R₁ is OR₃, R₂ is as originally defined, R₃ is an alkyl moiety, and Z₁ and Z₂ are methylene, can be prepared by treating compounds of Formula 8.6, wherein X, Y, R₁, R₂, R₃, Z₁ and Z₂ are as previously defined, with H₂ in the presence of a metal catalyst, such as Pd/C, in a polar solvent like EtOAc at a temperature of about 22° C. and a pressure of about 1 Atm, as shown in d. Compounds of Formula 8.8, wherein X and Y are tert-butoxycarbonyl, R₁ and R₂ are OR₃, R₃ is alkenyl, such as an allylic moiety, Z₁ is oxygen and Z₂ is methylene, can be prepared by subjecting compounds of Formula 8.0, wherein X, Y, R₁, and R₂ are as previously defined, Z₁ is oxygen, Z₂ is methylene, and R₃ is hydrogen, to the palladium mediated allylation conditions described in Step c, as shown in e. Compounds of Formula 8.9, wherein X and Y are tert-butoxycarbonyl, R₁ and R₂ are OR₃, R₃ is an alkyl moiety, Z₁ is oxygen and Z₂ is methylene, can be prepared by treating compounds of Formula 8.8, wherein X, Y, R₁, R₂, R₃, Z₁ and Z₂ are as previously defined, with H₂ in the presence of a metal catalyst, such as Pd/C, in a polar solvent like EtOAc at a temperature of about 40° C. and a pressure of about 600 p.s.i., as shown in f Compounds of Formula 8.0, wherein X is hydrogen, Y is tert-butoxycarbonyl, R₁ is OR₃, R₂ is as originally defined, R₃ is hydrogen, and Z₁ and Z₂ are methylene, can be treated with an amine base, such as N1,N1,N8,N8-tetramethylnaphthalene-1,8-diamine, and an alkylating agent, such as trimethyloxonium tetrafluoroborate (Meerwein salt), in an aprotic solvent like CH₂Cl₂ at about 0° C. to give compounds of Formula 8.10, wherein X, Y, R₁, R₂, Z₁ and Z₂ are as previously defined and R₃ is an alkyl group, e.g., methyl, as shown in g.

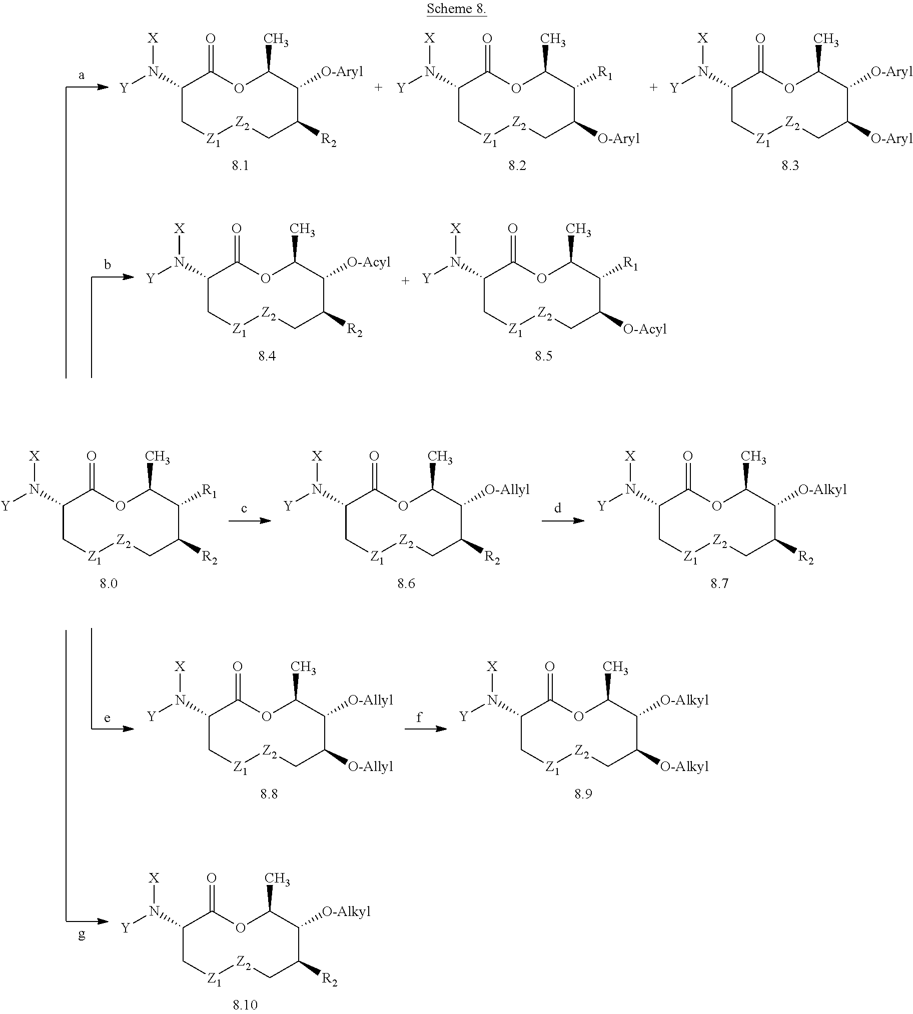

Compounds of Formula 9.10, wherein R₁ is set early in the synthesis and is as originally defined, but is not alkenyl or benzyl, R₂ is hydrogen, X is hydrogen, Y is tert-butoxycarbonyl, Z₁ is oxygen, and Z₂ is methylene, can be prepared according to the methods outlined in Scheme 9, Steps a-j. The compound of Formula 9.1, wherein R₁ is CH₂R₃ and R₃ is as originally defined, for example the propenyl moiety shown, can be prepared by treating the compound of Formula 9.0 with a strong base, such as lithium diisopropylamide (LDA), in a polar, aprotic solvent like THF, at a temperature between about −50° C. and −30° C., stirring at −30° C. for a period of about 1 h, and quenching the resulting lithium enolate with a solution of an electrophile, for example 1-bromo-3-methylbut-2-ene in a solvent like 1,2-dimethoxyethane, at about −78° C., as shown in a. The compound of Formula 9.2, wherein R₁ is CH₂R₃ and R₃ is the alkyl moiety shown, can be prepared from of the alkenyl compound of Formula 9.1, wherein R₁ is as previously defined, by treating with a metal catalyst, such as Pd/C, in a polar solvent like MeOH and reacting with H₂ at a temperature of about 22° C. and a pressure of about 1 Atm, as shown in b. The PMB protected alcohol of Formula 9.3, wherein R₁ is as defined above, can be prepared by treating the compound of Formula 9.2, wherein R₁ is as previously defined, with 4-methoxybenzyl 2,2,2-trichloroacetimidate in the presence of catalytic ((1S,4R)-7,7-dimethyl-2-oxobicyclo-[2.2.1]heptan-1-yl)methanesulfonic acid (camphorsulfonic acid, CSA) in an aprotic solvent like CH₂Cl₂ at a temperature between about 0° C. and 22° C., as shown in c. The aldehyde of Formula 9.4, wherein R₁ is as defined above, can be prepared from the ester of Formula 9.3, wherein R₁ is as previously defined, through a metal catalyzed hydrosilylation. For example, treating a mixture of the ester of Formula 9.3 and a metal catalyst, such as chlorobis(cyclooctene)iridium(I) dimer, with a reducing agent, such as diethylsilane (Et₂SiH₂), in a halogenated solvent like CH₂Cl₂ at about 0° C., as described by Cheng, C.; Brookhart, M. Angew. Chem. Int. Ed. 2012, 51, 9422-9424, affords the aldehyde of Formula 9.4, as shown in d. The aldehyde of Formula 9.4, wherein R₁ is as previously defined, can be treated with a nucleophile, such as a Grignard reagent like vinylmagnesium bromide, in a polar, aprotic solvent like THF at about −78° C. to give the alcohol of Formula 9.5, wherein R₁ is as defined above, as shown in e. The carbonate of Formula 9.6, wherein R₁ is as defined above, can be prepared by treating the alcohol of Formula 9.5, wherein R₁ is as previously defined, with a strong base, for example n-BuLi, in a polar, aprotic solvent like THF at about −78° C. and quenching the resultant anion with Boc₂O, as shown in f. The Bn ester of Formula 9.7, wherein R₁ is as defined above, can be prepared from the carbonate of Formula 9.6, wherein R₁ is as previously defined, through a metal catalyzed insertion of an alcohol into the olefin and subsequent displacement of the carbonate moiety. For example, treating a mixture of an alcohol, such as (S)-benzyl 2-((tert-butoxycarbonyl)amino)-3-hydroxypropanoate, a palladium catalyst, such as Pd₂(dba)₃, and a ligand, such as dppf, in a polar, aprotic solvent like THF with the carbonate of Formula 9.6, affords the ester of Formula 9.7, wherein R₁ is as previously defined, as shown in g. The alcohol of Formula 9.8, wherein R₁ is as defined above, can be prepared from the Bn ester of Formula 9.7, wherein R₁ is as previously defined, by treating with an oxidant, such as DDQ, in an aprotic solvent like CH₂Cl₂ at about 0° C., as shown in h. The seco acid of Formula 9.9, wherein R₁ is as defined above, can be prepared from the olefinic Bn ester of Formula 9.8, wherein R₁ is as previously defined, by treating with a metal catalyst, such as Pd/C, in a polar solvent like EtOAc and reacting with H₂ at a temperature of about 22° C. and a pressure of about 1 Atm, as shown in i. The macrocycle of Formula 9.10, wherein X, Y, R₁, R₂, R₃, Z₁ and Z₂ are as defined above, can be prepared from the seco acid of Formula 9.9, wherein R₁ is as previously defined, using the lactonization conditions described in Scheme 1, Step e, as shown in j.

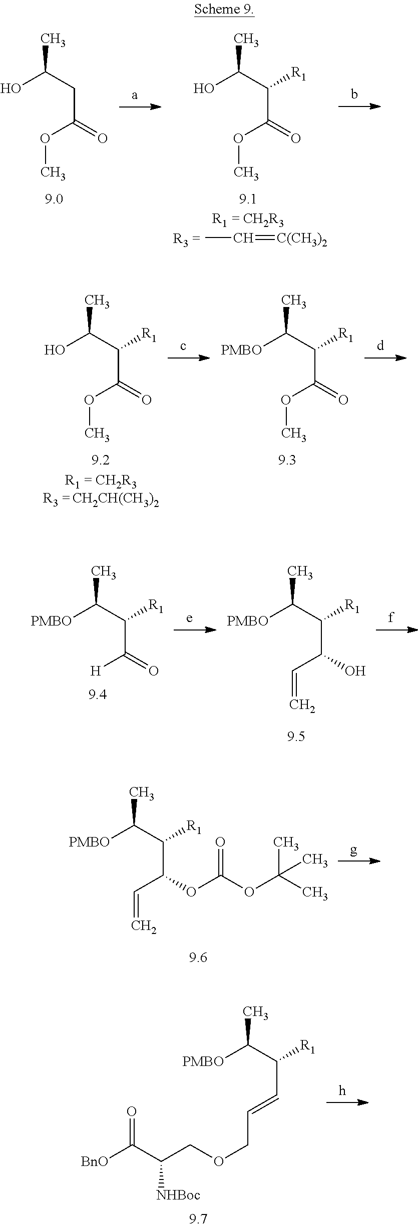

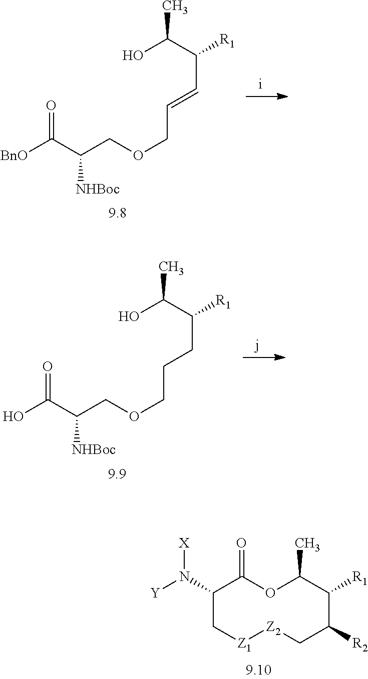

Macrocycles of Formula 10.6, wherein R₁ and R₂ are as originally defined, but not alkenyl, and are set early in the synthesis, can be prepared according to the methods outlined in Scheme 10, Steps a-f. For example, compounds of Formula 10.6, wherein R₁ is OR₃ and R₃ is alkyl, R₂ is CH₂R₃ and R₃ is aryl, X is hydrogen, Y is tert-butoxycarbonyl, Z₁ is methylene, and Z₂ is oxygen can be prepared using this method. Diols of Formula 10.0 (Meyer, K. G., et. al. Preparation of N-Macrocyclyl Picolinamides as fungicides U.S. Pat. No. 8,835,462, 2014) can be treated with a phase transfer catalyst, such as methyltributylammonium chloride, an aq solution of an alkali hydroxide base, such as NaOH, and an electrophile, such as 2-bromo-1,1-diethoxyethane, at about 110° C. to about 120° C. to give the compound of Formula 10.1, wherein R₁ and R₂ are as defined above, as shown in a. The acetal of Formula 10.1, wherein R₁ and R₂ are as previously defined, can be treated with an acid, such as 6 normal (N) aq hydrogen chloride (HCl), in an aprotic solvent like acetone to give the aldehyde of Formula 10.2, wherein R₁ and R₂ are as defined above, as shown in b. The Boc-protected alkenyl ether of Formula 10.3, wherein R₁ and R₂ are as defined above, can be prepared from the aldehyde of Formula 10.2, wherein R₁ and R₂ are as previously defined, using the Horner-Wadsworth-Emmons methodology described in Scheme 6, Step g, as shown in c. The Me ester of Formula 10.4, wherein R₁ and R₂ are as defined above, can be prepared from the alkenyl ether of Formula 10.3, wherein R₁ and R₂ are as previously defined, using slightly modified conditions of the asymmetric hydrogenation described in Scheme 5, Step d, i.e., the reaction was run in THF on a Paar shaker at 45 p.s.i., as shown in d. The seco acids of Formula 10.5, wherein R₁ and R₂ are as defined above, can be prepared from the esters of Formula 10.4, wherein R₁ and R₂ are as previously defined, using the saponification conditions described in Scheme 1, Step d, as shown in e. The macrocycle of Formula 10.6, wherein X, Y, R₁, R₂, Z₁ and Z₂ are as defined above, can be prepared from the seco acid of Formula 9.9, wherein R₁ and R₂ are as previously defined, using the lactonization conditions described in Scheme 1, Step e, as shown in f.

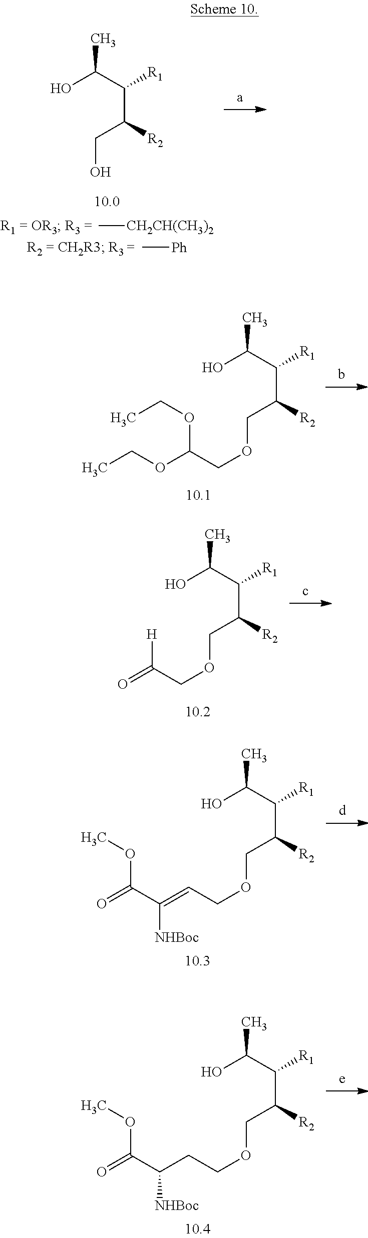

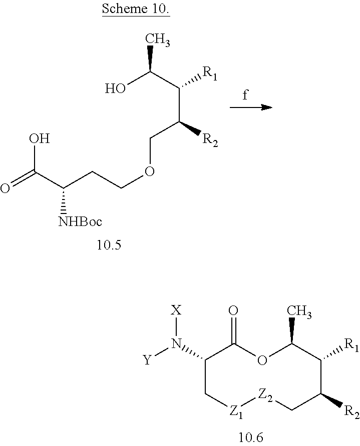

Compounds of Formulae 11.2 and 11.3 can be prepared through the methods shown in Scheme 11, Steps a-c. Compounds of Formula 11.2, wherein R₁, R₂, Z₁, Z₂ are as originally defined and X and Y are hydrogen, can be prepared from a variety of precursors, including, but not limited to, compounds of Formula 11.0, wherein R₁, R₂, Z₁, Z₂ are as originally defined and Y is tert-butoxycarbonyl, and compounds of Formula 11.1, wherein R₁, R₂, Z₁, Z₂ are as originally defined and X and Y are tert-butoxycarbonyl. Treating compounds of Formulas 11.0-11.1 with an acid, such as a 4.0 M HCl solution in dioxane, in a solvent such as CH₂Cl₂ affords the hydrochloride salt of compounds of Formula 11.2, which may be neutralized in situ in step c or neutralized prior to use to give the free amine, as shown in a. Alternatively, compounds of Formula 11.2, wherein R₁, R₂, Z₁, Z₂, X, and Y are as defined above, can be prepared from compounds of Formulas 11.0 and 11.1, wherein R₁, R₂, Z₁, Z₂, X, and Y are as previously defined, by treatment with TIPS-OTf in the presence of a base, such as 2,6-lutidine, in an aprotic solvent such as CH₂Cl₂, followed by treatment with a protic solvent such as MeOH, as shown in b. Compounds of Formula 11.3, wherein R₁, R₂, R₅, R₆, Z₁, and Z₂, are as originally defined, can be prepared from compounds of Formula 11.2, wherein R₁, R₂, Z₁, Z₂, X, and Y are as previously defined, by treatment with 3-hydroxypicolinic acid in the presence of an amine base, such as 4-methylmorpholine or NEt₃, and a peptide coupling reagent, such as O-(7-azabenzotriazol-1-yl)-N,N,N′,N′-tetramethyluronium hexafluorophosphate (HATU) or benzotriazol-1-yl-oxytripyrrolidinophosphonium hexafluorophosphate (PyBOP), in an aprotic solvent such as CH₂Cl₂, as shown in c.

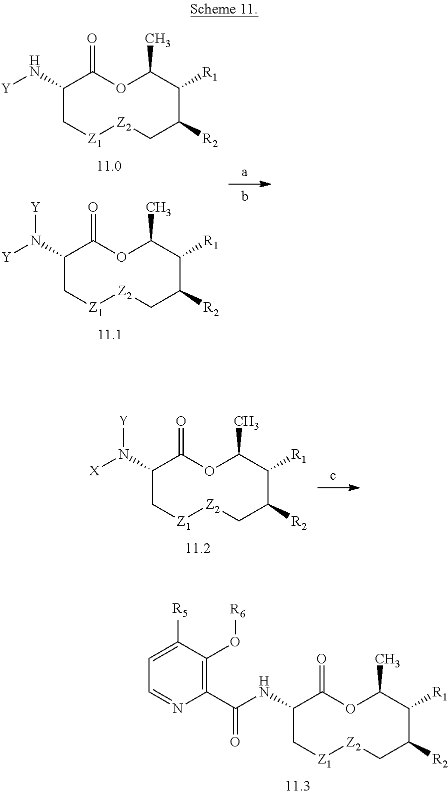

Compounds of Formula 12.0, wherein R₁, R₂, R₅, R₆, Z₁, and Z₂ are as originally defined, can be prepared by the method shown in Scheme 12. Compounds of Formula 12.0 can be prepared from compounds of Formula 11.3, wherein R₁, R₂, R₅, Z₁, and Z₂ are as previously defined and R₆ is hydrogen, by treatment with the appropriate alkyl halide with or without a reagent such as sodium iodide (NaI) and an alkali carbonate base such as sodium carbonate (Na₂CO₃) or K₂CO₃ in a solvent such as acetone or by treatment with an acyl halide in the presence of an amine base, such as pyridine, NEt₃, DMAP, or mixtures thereof, in an aprotic solvent such as CH₂Cl₂, as shown in a.

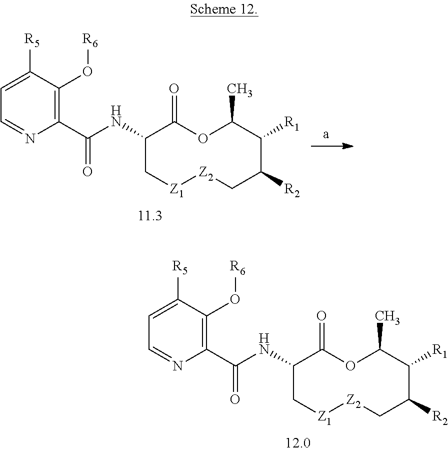

### EXAMPLES

### Example 1, Step 1: Preparation of (2S,3S,4S)-3,4-bis(benzyloxy)-2-methyl-3,4-dihydro-2H-pyran

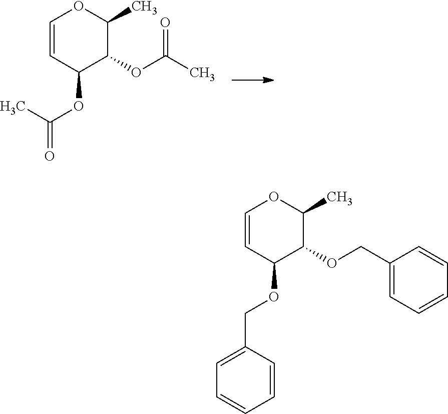 [SMILES: CC(=O)O[C@H]1[C@H](C)OC=C[C@@H]1OC(C)=O ; C[C@@H]1OC=C[C@H](OCc2ccccc2)[C@H]1OCc1ccccc1]

A suspension of (2S,3S,4S)-2-methyl-3,4-dihydro-2H-pyran-3,4-diyl diacetate (5.18 grams (g), 24.2 millimoles (mmol)) and K₂CO₃ (0.405 g, 2.93 mmol) in MeOH (25 milliliters (mL), 1 M was stirred at room temperature for 6.5 h. The solution was passed through a plug of silica gel (SiO₂) rinsing with EtOAc, and the filtrate was concentrated to give a white solid (3.02 g, 96%) that was used without purification. To a solution of the solid (2.80 g, 21.5 mmol) in DMF (42 mL, 0.5 M) at 0° C. (ice water bath) was added a 60% dispersion of NaH in mineral oil (2.15 g, 53.8 mmol) in four portions over a 20 minute (min) period. The resulting suspension was treated with BnBr (5.62 mL, 47.3 mmol) and the reaction mixture was stirred for 24 h while allowing to slowly warm to room temperature. The reaction mixture was carefully quenched with saturated (sat'd) aq ammonium chloride solution (NH₄Cl; 10 mL), diluted with EtOAc (50 mL), and washed with H₂O (2×25 mL). The organic phase was dried over sodium sulfate (Na₂SO₄), filtered, and concentrated to provide a yellow oil, which was purified by column chromatography (SiO₂, 0→50% EtOAc in hexanes) to give the title compound (5.99 g, 86%) as a clear, colorless oil: IR (Thin Film) 3063, 3030, 2870, 1645, 1453, 1236, 733, 696 (s) cm⁻¹; ¹H NMR (400 MHz, CDCl₃) δ 7.43-7.27 (m, 10H), 6.36 (dd, J=6.1, 1.3 Hz, 1H), 4.97-4.78 (m, 2H), 4.77-4.48 (m, 3H), 4.22 (ddd, J=6.5, 2.1, 1.5 Hz, 1H), 3.95 (dq, J=8.9, 6.4 Hz, 1H), 3.49 (dd, J=9.0, 6.5 Hz, 1H), 1.38 (d, J=6.4 Hz, 3H). ¹³C NMR (100 MHz, CDCl₃) δ 144.80, 138.41, 138.27, 128.42, 128.41, 127.97, 127.76, 127.64, 100.14, 79.53, 76.44, 74.07, 73.97, 70.54, 17.49.

### Example 1, Step 2: Preparation of (3S,4S,5S)-3,4-bis(benzyloxy)hexane-1,5-diol

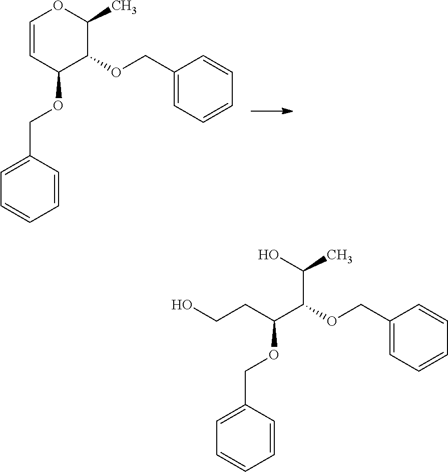 [SMILES: C[C@@H]1OC=C[C@H](OCc2ccccc2)[C@H]1OCc1ccccc1 ; C[C@H](O)[C@H](OCc1ccccc1)[C@H](CCO)OCc1ccccc1]

To a solution of (2S,3S,4S)-3,4-bis(benzyloxy)-2-methyl-3,4-dihydro-2H-pyran (5.59 g, 18.0 mmol) in THF (180 mL, 0.1 M) was added a solution of Hg(OAc)₂ (6.89 g, 21.6 mmol) in H₂O (90 mL) over a 7 min period via an addition funnel. The resulting clear, colorless solution was stirred at room temperature for 1 h, cooled to 0° C., and treated with NaBH₄ (2.04 g, 54.0 mmol) in portions over a 5 min period. The reaction mixture was stirred at 0° C. for 1 h, warmed to room temperature, and the majority of the THF and other volatile components were removed under reduced pressure. The residual aq was extracted with CH₂Cl₂ (200 mL, 2×100 mL) and the combined organic extracts were dried over Na₂SO₄, filtered, and concentrated to provide a gooey, white solid, which was shown to be a mixture of SM and product by ¹H NMR. The crude solid was dissolved in MeOH (90 mL), treated with NaBH₄ (2.00 g, 54.0 mmol) over a 10 min period, warmed to room temperature, stirred for 2 h, and quenched by pouring into ½ sat'd aq NH₄Cl solution (90 mL). The mixture was extracted with CH₂Cl₂ (90 mL, then 2×45 mL), and the combined organic extracts were dried over Na₂SO₄, filtered, and concentrated to provide a colorless oil, which was purified by column chromatography (SiO₂, 10→100% EtOAc in hexanes) to give the title compound (4.44 g, 75%) as a clear, colorless oil: IR (Thin Film) 3390, 3030, 2929, 2875, 1453, 1353, 1053, 735, 696 cm⁻¹; ¹H NMR (400 MHz, CDCl₃) δ 7.40-7.27 (m, 10H), 4.67 (d, J=11.3 Hz, 1H), 4.63 (s, 2H), 4.53 (d, J=11.3 Hz, 1H), 4.01 (p, J=6.0 Hz, 1H), 3.85 (dt, J=8.7, 4.4 Hz, 1H), 3.70 (dt, J=11.3, 5.8 Hz, 2H), 3.43 (dd, J=7.2, 4.6 Hz, 1H), 3.17 (s, 1H), 1.96 (dddd, J=14.5, 7.7, 5.2, 4.4 Hz, 1H), 1.89-1.76 (m, 2H), 1.26 (d, J=6.2 Hz, 3H); ¹³C NMR (101 MHz, CDCl₃) δ 137.90, 137.38, 128.64, 128.54, 128.32, 128.22, 128.07, 128.02, 81.19, 77.92, 73.74, 72.78, 67.56, 60.09, 32.55, 19.73.

### Example 1, Step 3: Preparation of (S)-methyl 3-((3S,4S,5S)-3,4-bis(benzyloxy)-5-hydroxyhexyloxy)-2-(tert-butoxycarbonylamino)propanoate

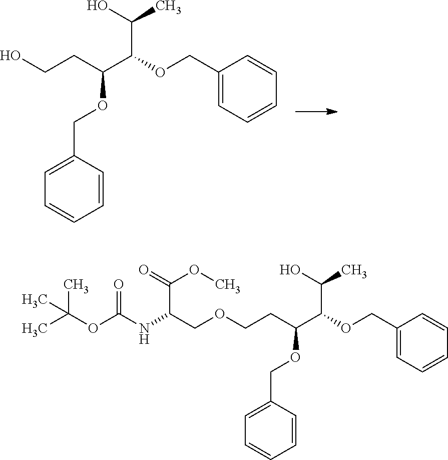 [SMILES: C[C@H](O)[C@H](OCc1ccccc1)[C@H](CCO)OCc1ccccc1 ; COC(=O)[C@H](COCC[C@H](OCc1ccccc1)[C@@H](OCc1ccccc1)[C@H](C)O)NC(=O)OC(C)(C)C]

To a solution of (S)-1-tert-butyl 2-methyl aziridine-1,2-dicarboxylate (1.06 g, 5.26 mmol) and (3S,4S,5S)-3,4-bis(benzyloxy)hexane-1,5-diol (3.48 g, 10.5 mmol) in CH₂Cl₂ (32 mL) at −78° C. (dry ice/acetone) was added BF₃.OEt₂ (130 microliters (μL), 1.05 mmol). The resulting solution was stirred at −78° C. for 1 h, warmed to 0° C. and stirred for 1 h, and quenched with ½ sat'd aq NaHCO₃ solution. The phases were separated and the aq phase was extracted with additional CH₂Cl₂ (2×30 mL). The combined organic extracts were dried over Na₂SO₄, filtered, and concentrated to provide a yellow oil, which was purified by column chromatography (SiO₂, 10→100% EtOAc in hexanes) to give the title compound (1.12 g, 40%) as a clear, colorless oil: IR (Thin Film) 3445, 2975, 2871, 1748, 1713, 1497, 1366, 1162, 1063, 733, 698 cm⁻¹; ¹H NMR (400 MHz, CDCl₃) δ 7.41-7.27 (m, 10H), 5.32 (d, J=8.6 Hz, 1H), 4.60 (m, 3H), 4.47 (d, J=11.4 Hz, 1H), 4.41 (d, J=8.7 Hz, 1H), 4.02-3.92 (m, 1H), 3.84-3.74 (m, 3H), 3.72 (s, 3H), 3.51 (dd, J=9.4, 3.3 Hz, 1H), 3.47 (dd, J=7.1, 5.1 Hz, 2H), 3.34 (dd, J=7.0, 4.6 Hz, 1H), 3.17 (d, J=3.7 Hz, 1H), 2.00-1.89 (m, 1H), 1.81-1.69 (m, 1H), 1.45 (s, 9H), 1.23 (d, J=6.4 Hz, 2H); ¹³C NMR (100 MHz, CDCl₃) δ 171.19, 155.46, 138.10, 137.62, 128.53, 128.47, 128.07, 81.14, 80.07, 77.22, 76.43, 73.46, 72.87, 70.72, 67.77, 67.49, 53.99, 52.47, 29.71, 28.33, 19.66; HRMS-ESI m/z [M+Na]⁺ calcd for C₂₉H₄₁O₈Na, 554.2724; found, 554.2711.

### Example 1, Step 4: (S)-3-((3S,4S,5S)-3,4-bis(benzyloxy)-5-hydroxyhexyloxy)-2-(tert-butoxycarbonylamino)propanoic acid

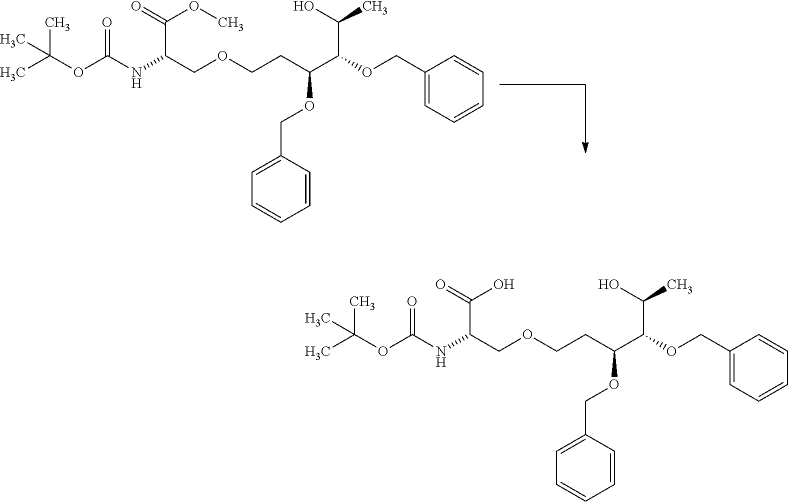

A solution of (S)-methyl 3-((3S,4S,5S)-3,4-bis(benzyloxy)-5-hydroxyhexyloxy)-2-(tert-butoxycarbonylamino)propanoate (3.24 g, 6.10 mmol) in THF (40 mL) and H₂O (20 mL) was treated with LiOH.H₂O (0.438 g, 18.3 mmol) and the reaction mixture was stirred for 15 h, poured into 1 N aq HCl (60 mL), and extracted with EtOAc (3×60 mL). The combined organic extracts were dried over magnesium sulfate (MgSO₄), filtered, and concentrated to provide the title compound (3.09 g, 98%) as a gooey, colorless material: ¹H NMR (400 MHz, CDCl₃) δ 7.38-7.26 (m, 10H), 5.37 (d, J=8.2 Hz, 1H), 4.61 (s, 2H), 4.57 (d, J=11.5 Hz, 1H), 4.48 (d, J=11.5 Hz, 1H), 4.41 (d, J=8.1 Hz, 1H), 3.99 (p, J=6.3 Hz, 1H), 3.80 (dd, J=9.1, 2.9 Hz, 1H), 3.77-3.71 (m, 1H), 3.51 (dd, J=9.2, 3.3 Hz, 1H), 3.49-3.41 (m, 2H), 3.36 (dd, J=6.4, 4.9 Hz, 1H), 1.93 (dtd, J=9.9, 7.5, 5.2 Hz, 1H), 1.75 (dt, J=13.9, 5.6 Hz, 1H), 1.44 (s, 9H), 1.22 (d, J=6.3 Hz, 5H); ¹³C NMR (101 MHz, CDCl₃) δ 173.90, 155.66, 138.17, 137.74, 128.51, 128.47, 128.33, 128.11, 128.02, 127.87, 81.38, 80.31, 73.77, 72.68, 70.58, 67.66, 67.59, 53.81, 29.61, 28.33, 19.29; ESIMS m/z 418.90 ([M+Na]⁺).

### Example 1, Step 5: Preparation of tert-butyl (3S,8R,9S,10S)-8,9-bis(benzyloxy)-10-methyl-2-oxo-1,5-dioxecan-3-ylcarbamate (Cmpd 181)

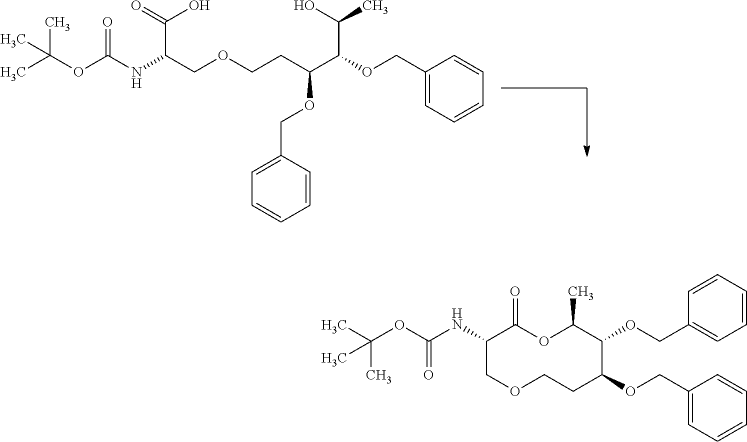

A solution of (S)-3-((3S,4S,5S)-3,4-bis(benzyloxy)-5-hydroxyhexyloxy)-2-(tert-butoxycarbonyl-amino)propanoic acid (2 g, 3.86 mmol) in toluene (42 mL) was added via a syringe pump over an 8 h period to a solution of MNBA (2.66 g, 7.73 mmol) and DMAP (2.83 g, 23.2 mmol) in toluene (1.25 L) at 45-50° C. with mechanical stirring. The mixture was stirred for an additional 4 h at 45-50° C., cooled to ambient temperature, and the liquid phase was decanted away from the precipitates and concentrated. The residue was purified by column chromatography (SiO₂, 5→40% acetone in hexanes) to give the title compound (776 milligrams (mg), 40%) as a hard white foam: ¹H NMR (400 MHz, CDCl₃) δ 7.37-7.19 (m, 10H), 5.59 (d, J=7.9 Hz, 1H), 5.32-5.22 (m, 1H), 5.02 (d, J=11.3 Hz, 1H), 4.91 (d, J=11.4 Hz, 1H), 4.56 (d, J=11.3 Hz, 1H), 4.52 (d, J=11.4 Hz, 1H), 4.48 (d, J=8.1 Hz, 1H), 3.78 (s, 2H), 3.63 (t, J=11.9 Hz, 1H), 3.58-3.52 (m, 1H), 3.36 (d, J=12.2 Hz, 1H), 3.28 (t, J=8.9 Hz, 1H), 2.11-2.00 (m, 1H), 1.60-1.51 (m, 1H), 1.44 (s, 9H), 1.31 (d, J=6.3 Hz, 3H); ¹³C NMR (100 MHz, CDCl₃) δ 169.74, 155.47, 138.81, 138.13, 128.39, 128.32, 128.07, 127.95, 127.76, 127.57, 84.60, 79.94, 77.94, 75.21, 75.18, 73.18, 67.37, 66.78, 55.34, 34.22, 28.31, 19.00; HRMS-ESI m/z [M+Na]⁺ calcd for C₂₈H₃₇NO₇Na, 522.2462; found, 522.2466.

### Example 1, Step 6: Preparation of tert-butyl N-tert-butoxycarbonyl-N-[(3S,8S,9S,10S)-8,9-dibenzyloxy-10-methyl-2-oxo-1,5-dioxecan-3-yl]carbamate (Cmpd 182)

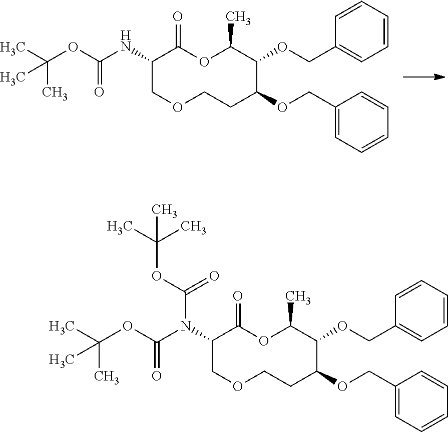 [SMILES: C[C@@H]1OC(=O)[C@@H](NC(=O)OC(C)(C)C)COCC[C@H](OCc2ccccc2)[C@H]1OCc1ccccc1 ; C[C@@H]1OC(=O)[C@@H](N(C(=O)OC(C)(C)C)C(=O)OC(C)(C)C)COCC[C@H](OCc2ccccc2)[C@H]1OCc1ccccc1]

To a solution of tert-butyl ((3S,8R,9S,10S)-8,9-bis(benzyloxy)-10-methyl-2-oxo-1,5-dioxecan-3-yl)carbamate (776 mg, 1.55 mmol) in CH₃CN (8 mL) were added DMAP (95 mg, 0.78 mmol) and Boc₂O (678 mg, 3.11 mmol) which resulted in gas evolution. The resulting solution was stirred at room temperature for 1 d, treated with additional Boc₂O (678 mg, 3.11 mmol) and DMAP (95 mg, 0.78 mmol), and stirred for an additional 24 h. The reaction mixture was concentrated and purified by column chromatography (SiO₂, 5→25% EtOAc in hexanes) to provide the title compound (286 mg, 31%): ¹H NMR (400 MHz, CDCl₃) δ 7.36-7.27 (m, 9H), 5.09 (dd, J=6.1, 2.3 Hz, 1H), 5.06 (dd, J=8.8, 6.3 Hz, 1H), 4.98 (d, J=11.2 Hz, 1H), 4.86 (d, J=11.4 Hz, 1H), 4.57 (d, J=11.2 Hz, 1H), 4.53 (d, J=11.5 Hz, 1H), 4.04-3.96 (m, 1H), 3.88 (dd, J=12.0, 6.1 Hz, 1H), 3.79-3.69 (m, 2H), 3.52-3.45 (m, 1H), 3.37 (t, J=8.4 Hz, 1H), 1.83-1.72 (m, 1H), 1.49 (s, 19H), 1.37 (d, J=6.3 Hz, 3H); ¹³C NMR (101 MHz, CDCl₃) δ 167.44, 152.62, 138.98, 138.23, 128.36, 128.32, 128.06, 127.75, 127.71, 127.47, 83.86, 82.81, 79.09, 77.22, 75.00, 74.56, 73.64, 69.84, 68.57, 59.10, 35.27, 27.92, 18.72.

### Example 1, Step 7: Preparation of tert-butyl N-tert-butoxycarbonyl-N-[(3S,8S,9R,10S)-8,9-dihydroxy-10-methyl-2-oxo-1,5-dioxecan-3-yl]carbamate (Cmpd 183)

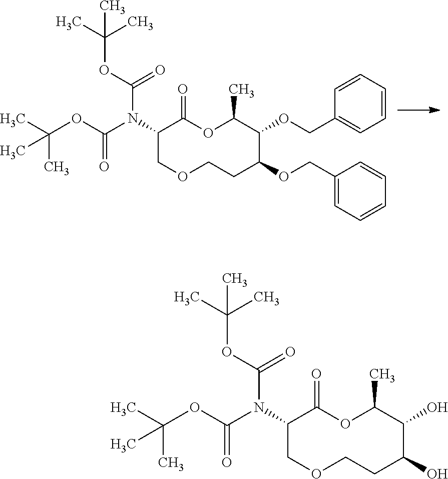 [SMILES: C[C@@H]1OC(=O)[C@@H](N(C(=O)OC(C)(C)C)C(=O)OC(C)(C)C)COCC[C@H](OCc2ccccc2)[C@H]1OCc1ccccc1 ; C[C@@H]1OC(=O)[C@@H](N(C(=O)OC(C)(C)C)C(=O)OC(C)(C)C)COCC[C@H](O)[C@H]1O]

To a solution of tert-butyl N-tert-butoxycarbonyl-N-[(3S,8S,9S,10S)-8,9-dibenzyloxy-10-methyl-2-oxo-1,5-dioxecan-3-yl]carbamate (191 mg, 0.32 mmol) in THF (10 mL) was added 10% Pd/C (50% H₂O, Degussa E101 NE/W; 68 mg, 0.032 mmol), and the resulting suspension was sealed in a stainless steel high-pressure reactor and pressurized to 600 psi with H₂. The reaction mixture was warmed to and stirred at 40° C. for 16 h, cooled to room temperature, filtered through a plug of Celite®, and concentrated to provide the title compound (134 mg, 100%) as a sticky oil: ¹H NMR (400 MHz, CDCl₃) δ 5.15 (dd, J=6.6, 1.8 Hz, 1H), 4.92 (dq, J=12.7, 6.3 Hz, 1H), 4.03 (dd, J=11.9, 1.8 Hz, 1H), 3.93-3.82 (m, 2H), 3.83-3.74 (m, 1H), 3.49 (ddd, J=11.8, 5.5, 3.1 Hz, 1H), 3.42 (td, J=8.2, 4.1 Hz, 1H), 3.02 (d, J=4.2 Hz, 1H), 2.35 (d, J=4.8 Hz, 1H), 2.15-2.03 (m, 1H), 1.69-1.60 (m, 1H), 1.59 (s, 3H), 1.50 (s, 19H), 1.44 (d, J=6.3 Hz, 4H); ¹³C NMR (101 MHz, CDCl₃) δ 167.36, 152.72, 82.92, 77.22, 75.42, 74.66, 70.07, 69.37, 68.37, 58.92, 35.41, 27.94, 18.78; HRMS-ESI m/z [M+Na]⁺ calcd for C₁₉H₃₃NNaO₉, 442.2048; found, 442.2049.

### Example 2, Step 1: Preparation of (2S,3S,4S)-3-(benzyloxy)-2-methyl-3,4-dihydro-2H-pyran-4-yl acetate

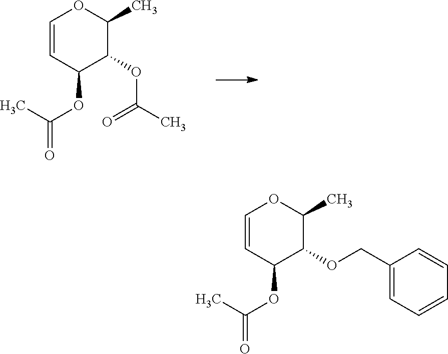 [SMILES: CC(=O)O[C@H]1[C@H](C)OC=C[C@@H]1OC(C)=O ; CC(=O)O[C@H]1C=CO[C@@H](C)[C@@H]1OCc1ccccc1]

A mixture of (2S,3S,4S)-2-methyl-3,4-dihydro-2H-pyran-3,4-diyl diacetate (24.5 g, 114 mmol), BnBr (29.9 mL, 252 mmol), NBu₄I (9.1 g, 24.6 mmol), and NaOH (50% aq, 91 g, 1100 mmol) was stirred under nitrogen (N₂) for 3 d. Another portion of NBu₄I (8.0 g, 22 mmol) was added and the mixture stirred for an additional 4 d. The reaction mixture was poured into H₂O (250 mL) and the phases were separated. The aq phase was extracted with CH₂Cl₂ (2×100 mL) and the combined organic phases were dried over Na₂SO₄, filtered, and concentrated to provide a yellow oil, which was purified by column chromatography (SiO₂, 2→25% acetone in hexanes) to give the title compound (19.58 g, 65%) as an oil: ¹H NMR (400 MHz, CDCl₃) δ 7.46-7.28 (m, 5H), 6.39 (dd, J=6.1, 1.3 Hz, 1H), 5.40 (ddd, J=6.2, 2.9, 1.4 Hz, 1H), 4.79-4.64 (m, 3H), 4.12-3.96 (m, 1H), 3.53 (dd, J=8.4, 6.3 Hz, 1H), 2.02 (s, 3H), 1.38 (d, J=6.5 Hz, 3H); ¹³C NMR (101 MHz, CDCl₃) δ 170.63, 145.81, 137.92, 128.45, 127.88, 99.17, 78.19, 73.95, 73.62, 71.00, 21.26, 17.25.

### Example 2, Step 2: Preparation of (2S,3R,4S)-3-(benzyloxy)-2-methyl-3,4-dihydro-2H-pyran-4-ol

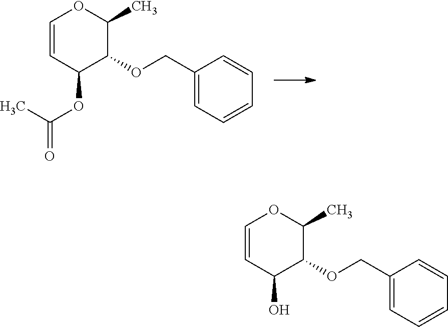 [SMILES: CC(=O)O[C@H]1C=CO[C@@H](C)[C@@H]1OCc1ccccc1 ; C[C@@H]1OC=C[C@H](O)[C@H]1OCc1ccccc1]

To a solution of (2S,3S,4S)-3-(benzyloxy)-2-methyl-3,4-dihydro-2H-pyran-4-yl acetate (19.5 g, 74.3 mmol) in MeOH (250 mL) was added K₂CO₃ (0.513 g, 3.71 mmol), and the resulting solution was stirred at room temperature for 5 h and filtered through a plug of SiO₂ to give the title compound (16.27 g, 99%) as a white, crystalline solid: ¹H NMR (400 MHz, CDCl₃) δ 7.41-7.26 (m, 5H), 6.30 (dd, J=6.0, 1.5 Hz, 1H), 4.83 (d, J=11.6 Hz, 1H), 4.77 (d, J=11.5 Hz, 1H), 4.68 (dd, J=6.0, 2.3 Hz, 1H), 4.38-4.29 (m, 1H), 3.89 (dq, J=9.6, 6.5 Hz, 1H), 3.27 (dd, J=9.6, 6.9 Hz, 1H), 1.95 (d, J=5.0 Hz, 1H), 1.40 (d, J=6.4 Hz, 3H); ¹³C NMR (101 MHz, CDCl₃) δ 144.59, 138.27, 128.58, 128.56, 127.97, 127.96, 103.22, 82.40, 74.24, 74.11, 69.96, 17.66.

### Example 2, Step 3: Preparation of (2S,3S,4S)-3-(benzyloxy)-4-butoxy-2-methyl-3,4-dihydro-2H-pyran

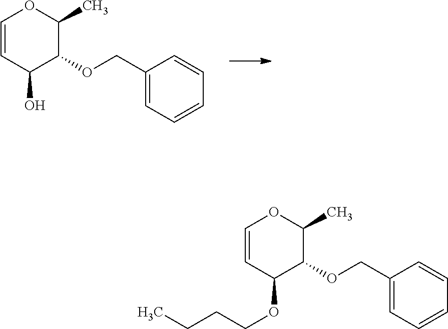 [SMILES: C[C@@H]1OC=C[C@H](O)[C@H]1OCc1ccccc1 ; CCCCO[C@H]1C=CO[C@@H](C)[C@@H]1OCc1ccccc1]

To a solution of (2S,3R,4S)-3-(benzyloxy)-2-methyl-3,4-dihydro-2H-pyran-4-ol (16.0 g, 72.6 mmol) in anhydrous DMF (291 mL) at 0° C. was added NaH (3.49 g, 145 mmol; 60% dispersion in mineral oil) in several portions over a 5 min period. The resulting slurry was stirred at 0° C. for 25 min, treated with 1-iodobutane (24.8 mL, 218 mmol), and allowed to warm to room temperature overnight. The reaction mixture was quenched with H₂O (10 mL), diluted with EtOAc (500 mL), and washed with H₂O (2×200 mL). The combined aq washes were extracted with EtOAc (2×200 mL), and the combined organic extracts were washed with sat'd aq sodium chloride (NaCl, brine; 2×200 mL), dried over MgSO₄, filtered, and concentrated to provide a yellow oil, which was purified by column chromatography (SiO₂, 0→30% acetone in hexanes) to give the title compound (14.8 g, 74%) as a yellow oil: ¹H NMR (400 MHz, CDCl₃) δ 7.45-7.27 (m, 5H), 6.34 (dd, J=6.1, 1.2 Hz, 1H), 4.89 (d, J=11.3 Hz, 1H), 4.82 (dd, J=6.1, 2.4 Hz, 1H), 4.70 (d, J=11.3 Hz, 1H), 4.05 (dt, J=6.6, 1.9 Hz, 1H), 3.92 (dq, J=9.0, 6.5 Hz, 1H), 3.61 (dt, J=9.1, 6.4 Hz, 1H), 3.53-3.42 (m, 1H), 3.39 (dd, J=9.1, 6.6 Hz, 1H), 1.70-1.51 (m, 2H), 1.47-1.38 (m, 2H), 1.36 (d, J=6.4 Hz, 3H), 0.93 (t, J=7.4 Hz, 3H); ¹³C NMR (101 MHz, CDCl₃) δ 144.51, 138.42, 128.39, 127.97, 127.72, 100.51, 79.46, 74.00, 73.93, 68.34, 32.29, 19.45, 17.53, 13.93.

### Example 3A: Preparation of 1-((2S,3R,4S)-4-(benzyloxy)-3-phenoxy-2-vinylpentyl)-4-fluorobenzene

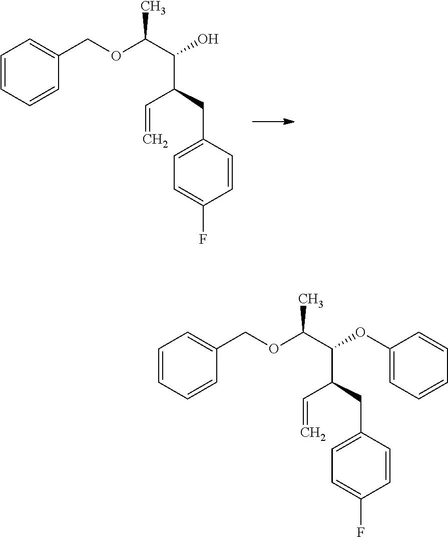 [SMILES: C=C[C@H](Cc1ccc(F)cc1)[C@@H](O)[C@H](C)OCc1ccccc1 ; C=C[C@H](Cc1ccc(F)cc1)[C@@H](Oc1ccccc1)[C@H](C)OCc1ccccc1]

To a solution of (2S,3R,4S)-2-(benzyloxy)-4-(4-fluorobenzyl)hex-5-en-3-ol (3.00 g, 9.54 mmol) in anhydrous toluene (48 mL) were added N-cyclohexyl-N-methylcyclohexanamine (3.04 mL, 14.3 mmol), Ph₃Bi(OAc)₂ (7.73 g, 14.3 mmol), and diacetoxycopper (0.347 g, 1.91 mmol), and the resulting blue suspension was heated to and stirred at 50° C. for 15 h. The reaction mixture was cooled to room temperature, filtered through a plug of Celite®, and concentrated to provide a blue suspension, which was purified by column chromatography (SiO₂, 1→5% EtOAc in hexanes) to give the title compound (2.77 g, 74%) as a clear, colorless oil: ¹H NMR (400 MHz, CDCl₃) δ 7.38-7.18 (m, 7H), 7.09-6.99 (m, 2H), 6.99-6.84 (m, 5H), 5.62 (dt, J=17.2, 9.7 Hz, 1H), 4.96 (dd, J=10.3, 1.7 Hz, 1H), 4.83 (d, J=17.2 Hz, 1H), 4.61 (d, J=11.6 Hz, 1H), 4.44 (d, J=11.6 Hz, 1H), 4.32 (t, J=5.5 Hz, 1H), 3.82 (p, J=6.1 Hz, 1H), 3.09 (dd, J=13.5, 4.1 Hz, 1H), 2.71 (dt, J=9.7, 5.0 Hz, 1H), 2.57 (dd, J=13.3, 9.8 Hz, 1H), 1.27 (d, J=6.2 Hz, 3H); ¹³C NMR (151 MHz, CDCl₃) δ 162.06, 160.45, 159.51, 138.48, 138.05, 135.87, 135.85, 130.84, 130.79, 129.44, 128.35, 127.67, 127.55, 120.92, 117.28, 116.31, 114.82, 114.68, 82.38, 75.70, 70.67, 48.55, 35.96, 15.11; ESIMS m/z 413.4 ([M+Na]⁺).

### Example 3B: Preparation of (2S,3R,4S)-2-(benzyloxy)-4-(4-fluorobenzyl)hex-5-en-3-yl isobutyrate

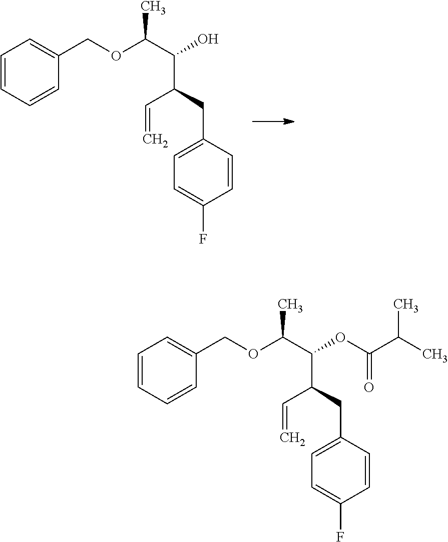 [SMILES: C=C[C@H](Cc1ccc(F)cc1)[C@@H](O)[C@H](C)OCc1ccccc1 ; C=C[C@H](Cc1ccc(F)cc1)[C@@H](OC(=O)C(C)C)[C@H](C)OCc1ccccc1]

To solution of (2S,3R,4S)-2-(benzyloxy)-4-(4-fluorobenzyl)hex-5-en-3-ol (3.00 g, 9.54 mmol), DMAP (1.75 g, 14.3 mmol), and NEt₃ (2.66 mL, 19.1 mmol) was added isobutyryl chloride (1.50 mL, 14.3 mmol) and the reaction mixture was stirred at room temperature for 20 h. The mixture was washed successively with 1 N HCl (40 mL), 0.1 N HCl (40 mL), and ½ sat'd aq NaHCO₃ (40 mL), dried over Na₂SO₄, filtered, and concentrated to provide the title compound (3.70 g, 96%) as a yellow oil: ¹H NMR (400 MHz, CDCl₃) δ 7.36-7.26 (m, 5H), 7.02 (ddd, J=8.3, 5.4, 2.5 Hz, 2H), 6.96-6.89 (m, 2H), 5.48 (ddd, J=17.2, 10.2, 9.2 Hz, 1H), 5.24 (dd, J=8.2, 4.0 Hz, 1H), 4.95 (dd, J=10.3, 1.6 Hz, 1H), 4.82-4.73 (m, 1H), 4.56 (d, J=11.6 Hz, 1H), 4.46 (d, J=11.6 Hz, 1H), 3.66 (qd, J=6.3, 4.0 Hz, 1H), 2.86 (dd, J=13.4, 3.5 Hz, 1H), 2.72-2.58 (m, 1H), 2.58-2.51 (m, 1H), 2.45 (dd, J=13.4, 10.2 Hz, 1H), 1.25-1.17 (m, 9H); ¹³C NMR (101 MHz, CDCl₃) δ 176.64, 161.29 (d, J=243.6 Hz), 138.46, 136.69, 135.30 (d, J=3.2 Hz), 130.67 (d, J=7.8 Hz), 128.31, 127.73, 127.50, 117.84, 114.81 (d, J=21.1 Hz), 74.49, 74.44, 70.46, 47.56, 36.21, 35.10, 34.35, 19.19, 19.16, 18.31, 14.38; ESIMS m/z 385.4 ([M+H]⁺).

### Example 3C: Preparation of ((((2S,3R,4S)-4-benzyl-3-isobutoxyhex-5-en-2-yl)oxy)methyl)-benzene

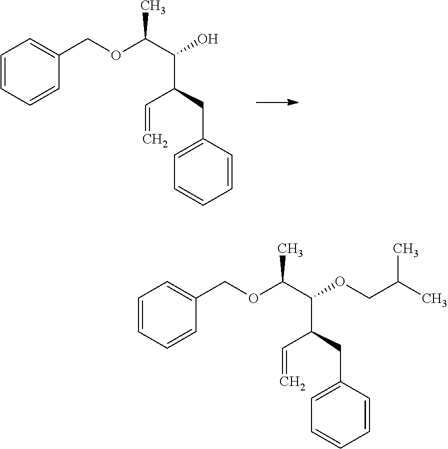 [SMILES: C=C[C@H](Cc1ccccc1)[C@@H](O)[C@H](C)OCc1ccccc1 ; C=C[C@H](Cc1ccccc1)[C@@H](OCC(C)C)[C@H](C)OCc1ccccc1]

To a solution of (2S,3R,4S)-4-benzyl-2-(benzyloxy)hex-5-en-3-ol (1.52 g, 5.13 mmol) in DMF (13 mL) were added isobutyl 4-methylbenzenesulfonate (2.93 g, 12.8 mmol) and KO^{t}Bu (1.44 g, 12.8 mmol), and the resulting dark green mixture was warmed to and stirred at 40° C. overnight. The reaction mixture was cooled to room temperature, quenched with H₂O, and extracted with diethyl ether (Et₂O; 3×). The combined organic extracts were washed with brine, dried over MgSO₄, filtered, and concentrated. The crude residue was purified by column chromatography (SiO₂, EtOAc in hexanes gradient) to afford the title compound (1.12 g, 62%) as a colorless oil: ¹H NMR (400 MHz, CDCl₃) δ 7.33 (d, J=4.4 Hz, 4H), 7.29-7.19 (m, 3H), 7.17-7.07 (m, 3H), 5.67-5.53 (m, 1H), 4.88 (dd, J=10.3, 1.9 Hz, 1H), 4.74 (dd, J=17.2, 1.9 Hz, 1H), 4.57 (d, J=11.7 Hz, 1H), 4.44 (d, J=11.8 Hz, 1H), 3.68-3.54 (m, 2H), 3.35-3.23 (m, 2H), 3.13 (q, J=8.9 Hz, 1H), 2.57-2.44 (m, 2H), 1.89 (m, 1H), 1.22 (d, J=6.2 Hz, 3H), 0.99-0.91 (m, 6H); ¹³C NMR (101 MHz, CDCl₃) δ 140.87, 139.14, 138.94, 129.45, 129.39, 128.29, 128.21, 127.91, 127.51, 127.37, 125.56, 116.35, 83.94, 79.37, 76.76, 70.53, 48.73, 36.87, 29.20, 19.68, 19.57, 14.38; ESIMS m/z 375.4 ([M+Na]⁺).

### Example 3D: Preparation of (((2S,3R,4S)-2-(benzyloxy)-4-vinylheptan-3-yl)oxy)triisopropyl-silane

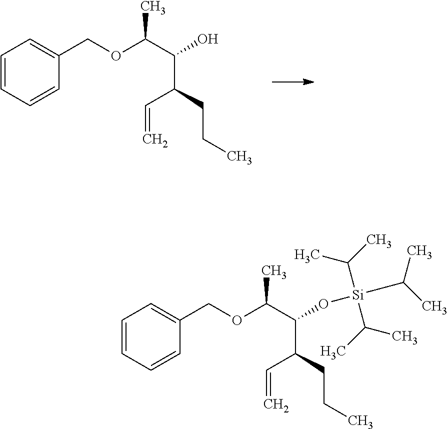 [SMILES: C=C[C@H](CCC)[C@@H](O)[C@H](C)OCc1ccccc1 ; C=C[C@H](CCC)[C@@H](O[Si](C(C)C)(C(C)C)C(C)C)[C@H](C)OCc1ccccc1]

To a solution of (2S,3R,4S)-2-(benzyloxy)-4-vinylheptan-3-ol (10.2 g, 41.1 mmol) in anhydrous CH₂Cl₂ (100 mL) at 0° C. were added 2,6-lutidine (6.22 mL, 53.4 mmol) and TIPS-OTf (13.4 mL, 49.3 mmol), and the reaction mixture was allowed to slowly warm to room temperature overnight. The mixture was poured into a well-stirred sat'd aq NaHCO₃ solution, stirred for 5 min, and the phases separated. The aq phase was extracted with CH₂Cl₂ (2×50 mL), and the combined organic phases were, dried over Na₂SO₄, filtered, and concentrated to provide a yellow oil, which was purified by column chromatography (SiO₂, 1-2% acetone in hexanes) to give the title compound (14.47 g, 87%) as a colorless oil: ¹H NMR (400 MHz, CDCl₃) δ 7.39-7.15 (m, 5H), 5.59 (ddd, J=17.2, 10.2, 9.1 Hz, 1H), 5.07-4.91 (m, 2H), 4.52 (d, J=11.8 Hz, 1H), 4.41 (d, J=11.8 Hz, 1H), 3.87 (dd, J=6.5, 2.8 Hz, 1H), 3.52 (qd, J=6.2, 2.9 Hz, 1H), 2.13 (dq, J=12.9, 5.0, 3.7 Hz, 1H), 1.62 (ddt, J=9.0, 5.9, 3.0 Hz, 1H), 1.35 (ddt, J=14.1, 7.2, 4.3 Hz, 1H), 1.28-1.19 (m, 1H), 1.19-1.15 (m, 4H), 1.15-1.10 (m, 3H), 1.09-1.04 (m, 18H), 0.86 (t, J=7.2 Hz, 3H); ¹³C NMR (101 MHz, CDCl₃) δ 139.71, 139.08, 128.13, 127.67, 127.23, 115.58, 78.23, 76.81, 70.50, 48.87, 32.74, 20.62, 18.43, 14.14, 13.76, 13.18; ESIMS m/z 427.4 ([M+Na]⁺).

### Example 4, Step 1: Preparation of (3S,4R,5S)-3-benzyl-5-(benzyloxy)-4-isobutoxyhexan-1-ol

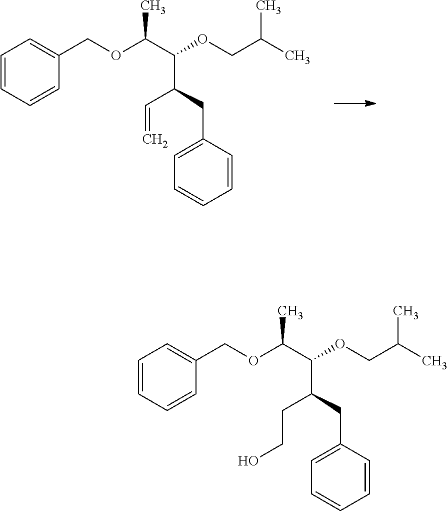 [SMILES: C=C[C@H](Cc1ccccc1)[C@@H](OCC(C)C)[C@H](C)OCc1ccccc1 ; CC(C)CO[C@H]([C@H](CCO)Cc1ccccc1)[C@H](C)OCc1ccccc1]

A round bottomed flask was charged with ((((2S,3R,4S)-4-benzyl-3-isobutoxyhex-5-en-2-yl)oxy)methyl)benzene (4.96 g, 14.1 mmol) and a solution of BH₃.THF (15.5 mL, 15.5 mmol, 1 M) was added at room temperature. The reaction mixture was allowed to stir for approximately 2 h, cooled to 0° C., and treated with 2N NaOH (30 mL) followed by H₂O₂ (5.80 mL, 56.3 mmol; 30%). The mixture was allowed to warm to room temperature overnight, carefully quenched by the addition of sat'd aq sodium bisulfite (NaHSO₃), and extracted with EtOAc (3×) The combined organics were washed with brine, dried over MgSO₄, filtered, and concentrated. The residue was purified by column chromatography (SiO₂, EtOAc in hexanes gradient) to afford the title compound (3.64 g, 70%) as a colorless oil: ¹H NMR (400 MHz, CDCl₃) δ 7.40-7.08 (m, 10H), 4.62 (d, J=11.4 Hz, 1H), 4.39 (d, J=11.4 Hz, 1H), 3.73 (dq, J=7.4, 6.1 Hz, 1H), 3.56 (ddt, J=16.7, 9.4, 5.6 Hz, 2H), 3.35-3.25 (m, 2H), 3.20 (dd, J=7.4, 2.6 Hz, 1H), 2.92 (dd, J=13.8, 5.7 Hz, 1H), 2.50 (dd, J=13.8, 9.2 Hz, 1H), 2.38-2.30 (m, 1H), 2.22-2.08 (m, 1H), 1.83 (dp, J=13.2, 6.6 Hz, 1H), 1.76-1.60 (m, 1H), 1.55 (dtd, J=14.3, 5.9, 4.2 Hz, 1H), 1.29 (d, J=6.0 Hz, 3H), 0.92 (dd, J=6.7, 1.5 Hz, 6H); ¹³C NMR (101 MHz, CDCl₃) δ 141.71, 138.57, 129.22, 128.38, 128.25, 127.86, 127.57, 125.75, 84.32, 75.84, 70.72, 62.03, 40.70, 37.87, 33.87, 29.07, 19.54, 19.52, 16.65; ESIMS m/z 393.3 ([M+Na]⁺).

### Example 4, Step 2A: Preparation of (S)-methyl 3-(((3S,4R,5S)-3-benzyl-5-(benzyloxy)-4-isobutoxyhexyl)oxy)-2-((tert-butoxycarbonyl)amino)propanoate

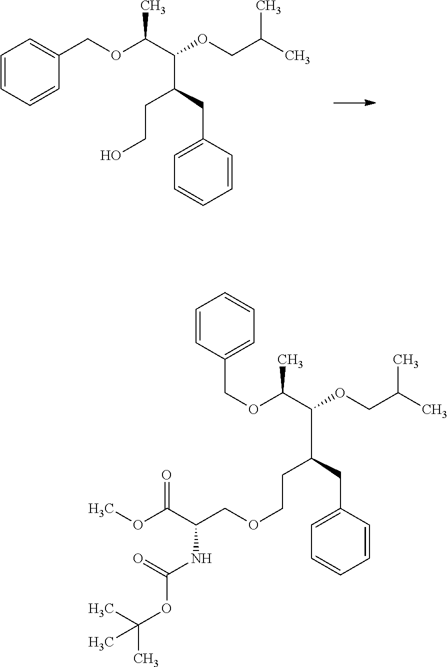

A round bottomed flask was charged with (S)-1-tert-butyl 2-methyl aziridine-1,2-dicarboxylate (74 mg, 0.37 mmol), (3S,4R,5S)-3-benzyl-5-(benzyloxy)-4-isobutoxyhexan-1-ol (139 mg, 0.376 mmol) and CH₂Cl₂ (1.8 mL) and the flask was briefly evacuated under vacuum and backfilled with N₂ (repeated 3×). The colorless solution was cooled to −78° C. in a dry ice/acetone bath and treated with BF₃.OEt₂ (7.0 μL, 0.059 mmol) to give a light-yellow solution. The reaction flask was moved to an ice bath and gradually warmed from 0° C. to room temperature over a 2 h period. The mixture was stirred at room temperature for an additional 3 h and quenched by the addition of 0.5 M aq sodium bisulfate (NaHSO₄) solution. The phases were separated and the aq phase was extracted with an additional portion of CH₂Cl₂. The combined organic phases were concentrated and the residue was purified by column chromatography (SiO₂) to afford the title compound (47.6 mg, 34% yield) as a colorless oil: ¹H NMR (400 MHz, CDCl₃) δ 7.38-7.05 (m, 10H), 5.45 (d, J=9.0 Hz, 1H), 4.63 (d, J=11.7 Hz, 1H), 4.37 (d, J=11.8 Hz, 1H), 4.35-4.29 (m, 1H), 3.73 (dd, J=9.4, 3.2 Hz, 1H), 3.68 (s, 3H), 3.60 (p, J=6.2 Hz, 1H), 3.44 (dd, J=9.4, 3.3 Hz, 1H), 3.37 (dd, J=8.6, 6.2 Hz, 1H), 3.31 (dt, J=6.6, 3.5 Hz, 2H), 3.23 (ddd, J=8.6, 5.6, 2.8 Hz, 2H), 2.91 (dd, J=13.9, 5.0 Hz, 1H), 2.42 (dd, J=13.9, 9.5 Hz, 1H), 2.19-2.07 (m, 1H), 1.83 (dp, J=13.2, 6.7 Hz, 1H), 1.70-1.56 (m, 1H), 1.53-1.47 (m, 1H), 1.45 (s, 9H), 1.28 (d, J=6.0 Hz, 3H), 0.92 (t, J=6.9 Hz, 6H); ¹³C NMR (101 MHz, CDCl₃) δ 171.25, 155.64, 141.83, 138.78, 129.22, 128.33, 128.16, 127.79, 127.47, 125.61, 84.12, 79.85, 79.32, 75.70, 70.62, 70.57, 54.07, 52.32, 39.18, 36.17, 30.55, 29.20, 28.35, 19.63, 19.52, 16.38.

### Example 4, Step 2B: Preparation of (S)-benzyl 3-(((3S,4R,5S)-5-(benzyloxy)-4-(cyclopropylmethoxy)-3-(4-fluorobenzyl)hexyl)oxy)-2-((tert-butoxycarbonyl)amino)propanoate

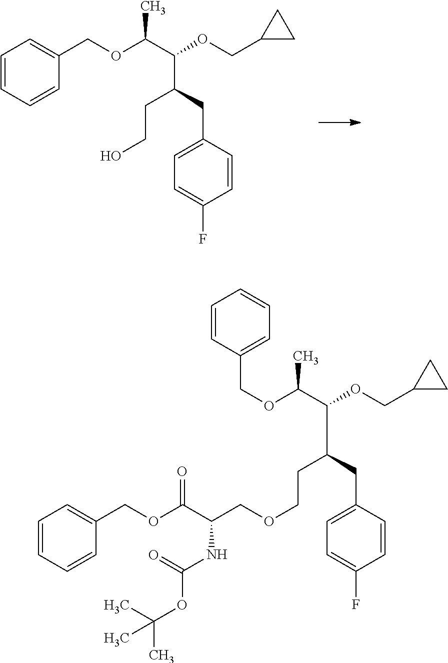 [SMILES: C[C@H](OCc1ccccc1)[C@H](OCC1CC1)[C@H](CCO)Cc1ccc(F)cc1 ; C[C@H](OCc1ccccc1)[C@H](OCC1CC1)[C@H](CCOC[C@H](NC(=O)OC(C)(C)C)C(=O)OCc1ccccc1)Cc1ccc(F)cc1]

To a solution of (3S,4R,5S)-5-(benzyloxy)-4-(cyclopropylmethoxy)-3-(4-fluorobenzyl)hexan-1-ol (2.72 g, 7.04 mmol) and (S)-2-benzyl 1-tert-butyl aziridine-1,2-dicarboxylate (2.15 g, 7.74 mmol) in CH₂Cl₂ (35 mL) was added BF₃.OEt₂ (0.089 mL, 0.70 mmol) at 0° C. under N₂, and the reaction mixture was stirred for 5 h and quenched by the addition of sat'd aq NaHCO₃. The phases were separated and the organics were dried by passing through a phase separator cartridge. The filtrate was concentrated to afford a crude oil which was purified by column chromatography (SiO₂, acetone in hexanes gradient) to afford the title compound (1.56 g, 33%) as a colorless oil: ¹H NMR (400 MHz, CDCl₃) δ 7.41-7.23 (m, 10H), 7.05-6.95 (m, 2H), 6.97-6.85 (m, 2H), 5.54 (d, J=8.8 Hz, 1H), 5.26-5.15 (m, 1H), 5.07 (d, J=12.5 Hz, 1H), 4.64 (d, J=11.7 Hz, 1H), 4.46-4.38 (m, 1H), 4.35 (d, J=11.7 Hz, 1H), 3.77 (dd, J=9.3, 3.1 Hz, 1H), 3.66-3.55 (m, 1H), 3.48 (dd, J=9.3, 3.2 Hz, 1H), 3.42-3.18 (m, 5H), 2.85 (dd, J=14.0, 5.0 Hz, 1H), 2.38 (dd, J=14.0, 9.6 Hz, 1H), 2.06 (dtd, J=10.1, 8.0, 4.6 Hz, 1H), 1.67-1.55 (m, 1H), 1.44 (s, 9H), 1.44-1.35 (m, 1H), 1.29 (d, J=6.0 Hz, 3H), 1.03 (dddd, J=11.5, 9.9, 5.0, 2.6 Hz, 1H), 0.54-0.43 (m, 2H), 0.21-0.12 (m, 2H); 19F NMR (376 MHz, CDCl₃) δ −117.90; ESIMS m/z 686.5 ([M+Na]⁺).

### Example 4, Step 3A: Preparation of (S)-methyl 3-(((3S,4R,5S)-3-benzyl-5-hydroxy-4-isobutoxyhexyl)oxy)-2-((tert-butoxycarbonyl)amino)propanoate

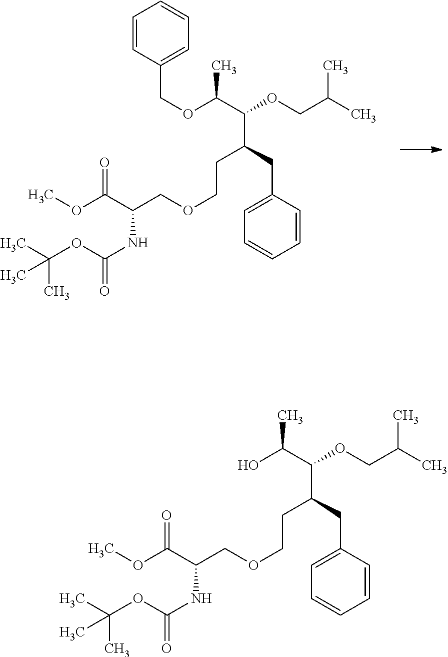

A round bottomed flask was charged with (S)-methyl 3-(((3S,4R,5S)-3-benzyl-5-(benzyloxy)-4-isobutoxyhexyl)oxy)-2-((tert-butoxycarbonyl)amino)propanoate (1.09 g, 1.91 mmol), EtOAc (9.5 mL), and 10% Pd/C (Degussa type, 50% H₂O, 0.203 g, 0.191 mmol), and the reaction flask was briefly evacuated under vacuum and backfilled with N₂. The flask was again evacuated under vacuum and was backfilled with H₂ (repeated 3×). The reaction mixture was placed under approximately 1 Atm of H₂ (balloon), stirred at room temperature for 3 h, filtered through a pad of Celite®, and concentrated. The crude oil was purified by column chromatography (SiO₂, EtOAc in hexanes gradient) to give the title compound (811 mg, 88%) as a colorless oil: ¹H NMR (400 MHz, CDCl₃) δ 7.34-7.22 (m, 2H), 7.23-7.11 (m, 3H), 5.47 (d, J=9.0 Hz, 1H), 4.36 (dt, J=9.1, 3.2 Hz, 1H), 3.96 (dt, J=11.9, 6.1 Hz, 1H), 3.79-3.70 (m, 1H), 3.73 (d, J=1.9 Hz, 3H), 3.47 (dd, J=9.4, 3.4 Hz, 1H), 3.38 (ddd, J=7.9, 6.1, 2.4 Hz, 2H), 3.27 (ddd, J=11.8, 8.9, 6.3 Hz, 2H), 3.16 (dd, J=6.0, 3.3 Hz, 1H), 3.06 (dd, J=13.8, 4.3 Hz, 1H), 2.41 (dd, J=13.9, 10.2 Hz, 1H), 2.21-2.09 (m, 1H), 1.92 (d, J=5.1 Hz, 1H), 1.90-1.80 (m, 1H), 1.66-1.51 (m, 2H), 1.46 (d, J=3.5 Hz, 9H), 1.28 (d, J=6.2 Hz, 3H), 1.00-0.89 (m, 6H); ¹³C NMR (101 MHz, CDCl₃) δ 171.39, 155.54, 141.60, 129.12, 128.27, 125.74, 84.72, 79.97, 79.16, 70.60, 70.00, 68.12, 53.98, 52.44, 38.59, 35.95, 30.14, 29.21, 28.35, 19.57, 19.48; HRMS-ESI (m/z) [M+H]⁺ calcd for C₂₆H₄₃NO₇, 481.304; found, 481.3046.

### Example 4 Step 3B: Preparation of (S)-2-((tert-butoxycarbonyl)amino)-3-(((3S,4R,5S)-4-(cyclopropylmethoxy)-3-(4-fluorobenzyl)-5-hydroxyhexyl)oxy)propanoic acid

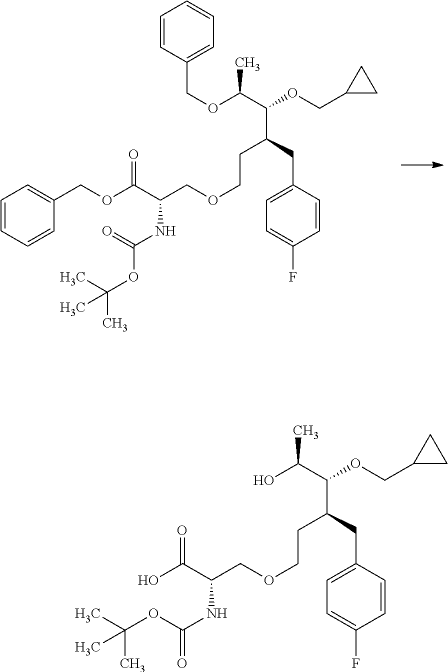 [SMILES: C[C@H](OCc1ccccc1)[C@H](OCC1CC1)[C@H](CCOC[C@H](NC(=O)OC(C)(C)C)C(=O)OCc1ccccc1)Cc1ccc(F)cc1 ; C[C@H](O)[C@H](OCC1CC1)[C@H](CCOC[C@H](NC(=O)OC(C)(C)C)C(=O)O)Cc1ccc(F)cc1]

To a solution of (S)-benzyl 3-(((3S,4R,5R)-5-(benzyloxy)-4-(cyclopropylmethoxy)-3-(4-fluorobenzyl)hexyl)oxy)-2-((tert-butoxycarbonyl)amino)propanoate (1.56 g, 2.35 mmol) in EtOAc (12 mL) was added Pd/C (10 wt %, 0.125 g, 0.118 mmol) and the reaction flask was fitted with a rubber septum. The flask was briefly evacuated under vacuum and backfilled with N₂ and then the evacuation under vacuum and backfill was repeated with H₂ (3×). The reaction mixture was placed under approximately 1 Atm of H₂ (balloon), stirred at room temperature overnight, filtered through a plug of Celite®, and concentrated to give the title compound (1.14 g, 100%) as a white solid: ¹H NMR (500 MHz, CDCl₃) δ 7.13 (ddd, J=8.9, 5.4, 2.7 Hz, 2H), 7.01-6.91 (m, 2H), 5.47-5.34 (m, 1H), 5.24 (s, 2H), 4.45-4.35 (m, 1H), 3.95 (p, J=6.3 Hz, 1H), 3.79 (dd, J=9.4, 3.2 Hz, 1H), 3.58 (dd, J=9.1, 3.1 Hz, 1H), 3.49-3.31 (m, 4H), 3.18 (dd, J=6.7, 3.5 Hz, 1H), 3.00 (dd, J=14.1, 4.4 Hz, 1H), 2.38 (dd, J=14.1, 10.4 Hz, 1H), 2.23-2.13 (m, 1H), 1.59-1.48 (m, 2H), 1.45 (d, J=3.1 Hz, 9H), 1.28 (d, J=6.2 Hz, 3H), 1.07 (dddd, J=13.3, 6.8, 5.0, 2.6 Hz, 1H), 0.57-0.46 (m, 2H), 0.20 (dt, J=5.8, 4.5 Hz, 2H); ¹⁹F NMR (471 MHz, CDCl₃) δ −117.68; ESIMS m/z 484.4 ([M+H]⁺).

### Example 4, Step 4: Preparation of (S)-3-(((3S,4R,5S)-3-benzyl-5-hydroxy-4-isobutoxyhexyl)oxy)-2-((tert-butoxycarbonyl)amino)propanoic acid

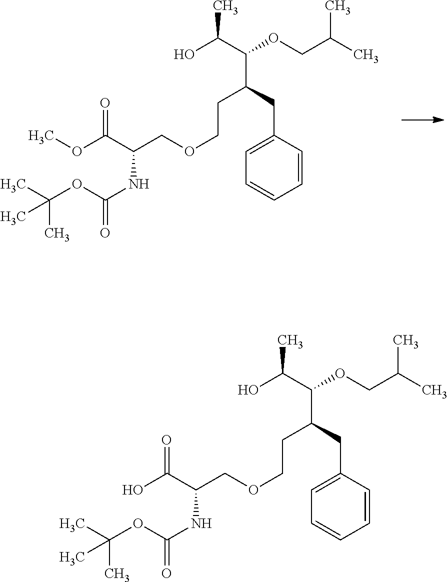

A round bottomed flask was charged with (S)-methyl 3-(((3S,4R,5S)-3-benzyl-5-hydroxy-4-isobutoxyhexyl)oxy)-2-((tert-butoxycarbonyl)amino)propanoate (811 mg, 1.68 mmol), THF (6.3 mL), H₂O (2.1 mL) and LiOH.H₂O (212 mg, 5.05 mmol), and the biphasic mixture was stirred at room temperature for approximately 2 h. The reaction mixture was quenched by the addition of 1N aq HCl, extracted with CH₂Cl₂ (3×), and the combined organic extracts were dried by passing through a phase separator cartridge and concentrated to give the title compound (596 mg, 76%) as a sticky, colorless oil: ¹H NMR (400 MHz, CDCl₃) δ 7.30-7.25 (m, 2H), 7.20-7.15 (m, 3H), 5.93 (s, 1H), 5.46 (d, J=8.3 Hz, 1H), 4.41 (d, J=8.3 Hz, 1H), 3.98-3.82 (m, 1H), 3.58 (dd, J=9.4, 3.1 Hz, 1H), 3.41 (ddd, J=8.5, 5.9, 4.0 Hz, 3H), 3.28-3.21 (m, 1H), 3.16 (dd, J=6.6, 3.3 Hz, 1H), 3.06-2.97 (m, 1H), 2.41 (dd, J=13.9, 9.9 Hz, 1H), 2.22 (dq, J=9.9, 5.2 Hz, 1H), 1.60-1.52 (m, 2H), 1.45 (d, J=1.8 Hz, 9H), 1.45 (s, 1H), 1.26 (d, J=6.3 Hz, 3H), 0.99-0.89 (m, 7H); ¹³C NMR (101 MHz, CDCl₃) δ 173.35, 155.62, 141.47, 129.12, 128.35, 125.81, 84.24, 80.07, 79.84, 70.93, 69.00, 68.71, 53.99, 37.97, 35.48, 30.22, 29.28, 28.34, 19.57, 19.47; ESIMS m/z 466.4 ([M−H]⁻).

### Example 5, Step 1: Preparation of (3S,4R,5S)-5-(benzyloxy)-3-propyl-4-((triisopropylsilyl)-oxy)hexanal

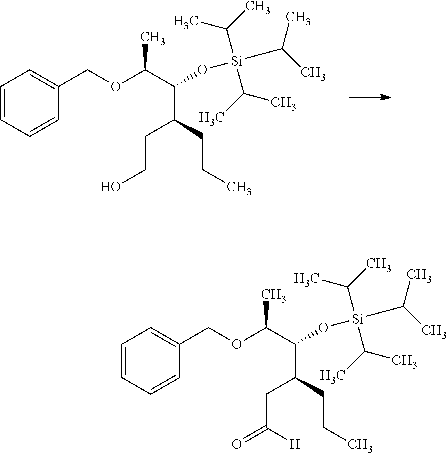 [SMILES: CCC[C@@H](CCO)[C@@H](O[Si](C(C)C)(C(C)C)C(C)C)[C@H](C)OCc1ccccc1 ; CCC[C@@H](CC=O)[C@@H](O[Si](C(C)C)(C(C)C)C(C)C)[C@H](C)OCc1ccccc1]

A solution of (3S,4R,5S)-5-(benzyloxy)-3-propyl-4-((triisopropylsilyl)oxy)hexan-1-ol (10.4 g, 24.6 mmol) in anhydrous CH₂Cl₂ (51 mL) and DMSO (10 mL) was cooled to 0° C. and treated with NEt₃ (10.29 mL, 73.8 mmol) followed by SO₃.pyridine complex (5.87 g, 36.9 mmol) in portions over a 5 min period. The reaction mixture was stirred at 0° C. and allowed to warm to room temperature overnight as the ice in the cooling bath melted. The mixture was concentrated and the residue was purified by column chromatography (SiO₂, 2→16% acetone in hexanes) to give the title compound (9.26 g, 89%) as a clear, colorless oil: ¹H NMR (400 MHz, CDCl₃) δ 9.72 (dd, J=2.8, 1.7 Hz, 1H), 7.37-7.22 (m, 5H), 4.57 (d, J=11.8 Hz, 1H), 4.40 (d, J=11.8 Hz, 1H), 4.02 (t, J=3.8 Hz, 1H), 3.51 (qd, J=6.2, 3.9 Hz, 1H), 2.68 (ddd, J=16.1, 4.7, 1.7 Hz, 1H), 2.41-2.28 (m, 1H), 2.19 (ddd, J=16.1, 8.5, 2.9 Hz, 1H), 1.46-1.35 (m, 1H), 1.35-1.25 (m, 3H), 1.22 (d, J=6.2 Hz, 3H), 1.16-1.00 (m, 21H), 0.85 (t, J=7.0 Hz, 3H); ¹³C NMR (101 MHz, CDCl₃) δ 202.94, 138.70, 128.23, 127.61, 127.39, 76.67, 75.99, 45.23, 39.10, 34.28, 21.06, 18.26, 15.98, 14.33, 12.91; ESIMS m/z 443.3 ([M+Na]⁺).

### Example 5, Step 2: Preparation of (((2S,3R,4S)-2-(benzyloxy)-4-propylhept-6-en-3-yl)oxy)triisopropylsilane

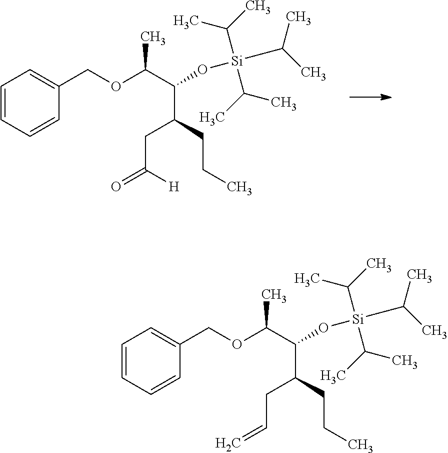 [SMILES: CCC[C@@H](CC=O)[C@@H](O[Si](C(C)C)(C(C)C)C(C)C)[C@H](C)OCc1ccccc1 ; C=CC[C@H](CCC)[C@@H](O[Si](C(C)C)(C(C)C)C(C)C)[C@H](C)OCc1ccccc1]

To a suspension of methyltriphenylphosphonium bromide (8.98 g, 25.1 mmol) in anhydrous THF (100 mL) was added KO^{t}Bu (2.70 g, 24.1 mmol) as a solid, and the resulting yellow suspension was stirred at room temperature for 30 min, cooled to 0° C. (ice water bath), and treated with a solution of (3S,4R,5S)-5-(benzyloxy)-3-propyl-4-((triisopropylsilyl)oxy)hexanal (9.2 g, 21.9 mmol) in anhydrous THF (10 mL). The reaction mixture was stirred at 0° C. for 5 min, removed from the cold bath, stirred at room temperature for 3 h, poured into brine (100 mL), and extracted with EtOAc (50 mL and 100 mL). The combined organic extracts were dried over MgSO₄, filtered, and concentrated to provide an oily, white solid, which was purified by column chromatography (SiO₂, 1→5% acetone in hexanes) to provide the title compound (7.06 g, 77%) as a clear, colorless oil: ¹H NMR (400 MHz, CDCl₃) δ 7.35-7.21 (m, 5H), 5.73 (dddd, J=16.7, 10.6, 7.8, 6.2 Hz, 1H), 5.01-4.96 (m, 1H), 4.95 (s, 1H), 4.55 (d, J=11.9 Hz, 1H), 4.43 (d, J=11.9 Hz, 1H), 3.98 (t, J=3.8 Hz, 1H), 3.55 (qd, J=6.2, 3.5 Hz, 1H), 2.47-2.32 (m, 1H), 1.87-1.71 (m, 1H), 1.63 (ddt, J=8.5, 6.4, 3.0 Hz, 1H), 1.41-1.25 (m, 4H), 1.21 (d, J=6.2 Hz, 3H), 1.08 (d, J=2.8 Hz, 21H), 0.84 (t, J=7.0 Hz, 3H); ¹³C NMR (101 MHz, CDCl₃) δ 139.06, 138.40, 128.16, 127.54, 127.23, 115.54, 76.60, 76.12, 70.33, 43.20, 34.71, 32.77, 21.29, 18.34, 15.62, 14.51, 13.01; ESIMS m/z 441.4 ([M+Na]⁺).

### Example 5, Step 3: Preparation of (7S,8R,9S,Z)-benzyl 9-(benzyloxy)-2-((tert-butoxycarbonyl)amino)-7-propyl-8-((triisopropylsilyl)oxy)dec-2-enoate

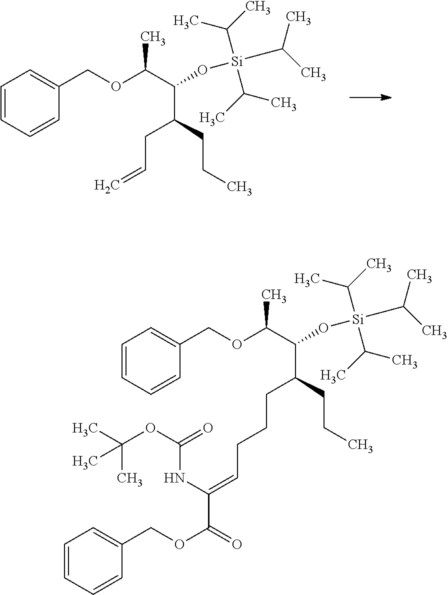 [SMILES: C=CC[C@H](CCC)[C@@H](O[Si](C(C)C)(C(C)C)C(C)C)[C@H](C)OCc1ccccc1 ; CCC[C@@H](CCC/C=C(\NC(=O)OC(C)(C)C)C(=O)OCc1ccccc1)[C@@H](O[Si](C(C)C)(C(C)C)C(C)C)[C@H](C)OCc1ccccc1]

To a 250 mL, oven-dried Schlenk flask were added (((2S,3R,4S)-2-(benzyloxy)-4-propylhept-6-en-3-yl)oxy)triisopropylsilane (7.01 g, 16.7 mmol) and a 0.5 M solution of 9-BBN in THF (50.2 mL, 25.1 mmol) under N₂, and the resulting solution was stirred at room temperature for 5 h. The mixture was treated with K₃PO₄ (3 M in H₂O, 10.0 mL, 30.1 mmol) followed by a solution of (Z)-benzyl 3-bromo-2-((tert-butoxycarbonyl)amino)acrylate (5.96 g, 16.7 mmol) in DMF (28 mL). The mixture was degassed by evacuating under vacuum and backfilling with N₂ (3×) and then treated with PdCl₂(dppf).CH₂Cl₂ adduct (0.684 g, 0.837 mmol). Following the catalyst addition, the degassing protocol was repeated (3×) and the reaction mixture was warmed to and stirred at 60° C. for 7 h. The mixture was cooled to room temperature, diluted with Et₂O (200 mL), washed with brine (100 mL), dried over MgSO₄, filtered, and concentrated to provide a brown oil, which was purified by iterative column chromatography (SiO₂, 1→5% methyl tert-butyl ether (MTBE) in CH₂Cl₂; SiO₂, 2→10% EtOAc in hexanes; SiO₂, 1→10% acetone in toluene) to provide the title compound (9.31 g, 80%) as a clear, colorless oil: ¹H NMR (400 MHz, CDCl₃) δ 7.40-7.33 (m, 5H), 7.31 (d, J=4.4 Hz, 4H), 7.28-7.21 (m, 1H), 6.59 (t, J=7.3 Hz, 1H), 5.92 (s, 1H), 5.20 (s, 2H), 4.54 (d, J=11.9 Hz, 1H), 4.40 (d, J=11.9 Hz, 1H), 3.91 (t, J=3.8 Hz, 1H), 3.50 (qd, J=6.2, 3.7 Hz, 1H), 2.16 (q, J=7.4 Hz, 2H), 1.58-1.46 (m, 3H), 1.44 (s, 9H), 1.41-1.33 (m, 2H), 1.33-1.26 (m, 2H), 1.25-1.21 (m, 1H), 1.19 (d, J=6.2 Hz, 3H), 1.07 (s, 22H), 0.84 (t, J=7.0 Hz, 3H); ¹³C NMR (101 MHz, CDCl₃) δ 164.81, 153.30, 139.03, 137.42, 135.71, 129.04, 128.54, 128.28, 128.26, 128.23, 128.17, 127.58, 127.25, 125.30, 80.44, 76.16, 70.35, 67.04, 43.38, 33.20, 30.28, 29.06, 28.20, 26.96, 21.46, 18.35, 15.68, 14.59, 13.06; ESIMS m/z 718.5 ([M+Na]⁺).

### Example 5, Step 4: Preparation of (2S,7S,8R,9S)-benzyl 9-(benzyloxy)-2-((tert-butoxycarbonyl)amino)-7-propyl-8-((triisopropylsilyl)oxy)decanoate

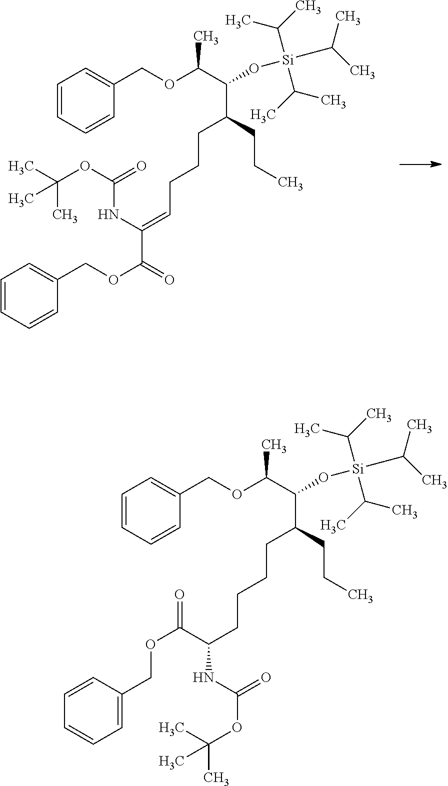 [SMILES: CCC[C@@H](CCC/C=C(\NC(=O)OC(C)(C)C)C(=O)OCc1ccccc1)[C@@H](O[Si](C(C)C)(C(C)C)C(C)C)[C@H](C)OCc1ccccc1 ; CCC[C@@H](CCCC[C@H](NC(=O)OC(C)(C)C)C(=O)OCc1ccccc1)[C@@H](O[Si](C(C)C)(C(C)C)C(C)C)[C@H](C)OCc1ccccc1]

A solution of (7S,8R,9S,Z)-benzyl 9-(benzyloxy)-2-((tert-butoxycarbonyl)amino)-7-propyl-8-((triisopropylsilyl)oxy)dec-2-enoate (9.24 g, 13.2 mmol) in MeOH (44 mL) was sparged with N₂ for 10 min, treated with (S,S)-Et-Rh-Duphos (0.144 g, 0.199 mmol), and the sparging continued for an additional 5 min following the addition. The solution was sealed in a stainless steel reactor and the reactor was pressurized to 200 psi with H₂. The reaction mixture was stirred at room temperature for 20 h, concentrated, and purified by column chromatography (SiO₂, 2→20% acetone in hexanes) to provide the title compound (8.28 g, 89%) as a clear, colorless oil: ¹H NMR (400 MHz, CDCl₃) δ 7.43-7.21 (m, 10H), 5.20 (d, J=12.4 Hz, 1H), 5.12 (d, J=12.4 Hz, 1H), 4.98 (d, J=8.3 Hz, 1H), 4.54 (d, J=11.9 Hz, 1H), 4.41 (d, J=11.9 Hz, 1H), 4.31 (q, J=7.5 Hz, 1H), 3.91 (t, J=3.6 Hz, 1H), 3.48 (qd, J=6.2, 3.6 Hz, 1H), 1.86-1.71 (m, 1H), 1.64-1.53 (m, 1H), 1.44 (s, 11H), 1.37-1.23 (m, 6H), 1.21 (d, J=2.6 Hz, 1H), 1.19 (d, J=6.2 Hz, 5H), 1.07 (s, 21H), 0.84 (t, J=7.0 Hz, 3H); ¹³C NMR (101 MHz, CDCl₃) δ 172.83, 155.36, 139.06, 135.50, 128.57, 128.37, 128.25, 128.17, 127.55, 127.24, 79.80, 76.10, 70.30, 66.91, 53.60, 43.48, 33.26, 32.69, 30.10, 28.34, 27.87, 25.90, 21.45, 18.35, 15.66, 14.61, 13.06; ESIMS m/z 698.4 ([M+H]⁺).

### Example 5, Step 5: Preparation of (7S,8R,9S)-2-((tert-butoxycarbonyl)amino)-9-hydroxy-7-propyl-8-((triisopropyl silyl)oxy)decanoic acid

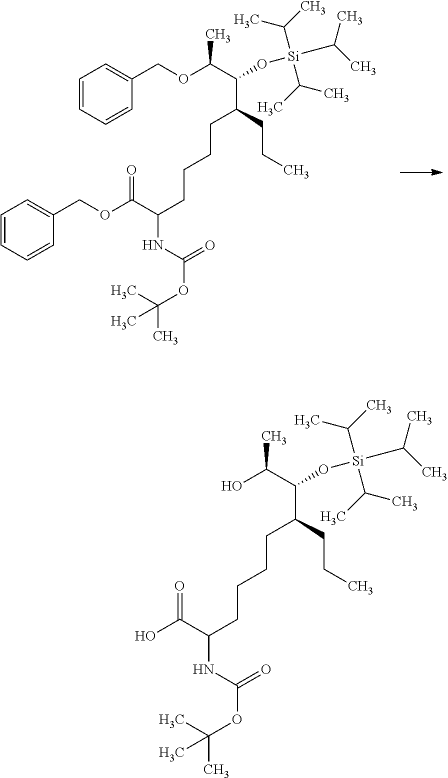 [SMILES: CCC[C@@H](CCCCC(NC(=O)OC(C)(C)C)C(=O)OCc1ccccc1)[C@@H](O[Si](C(C)C)(C(C)C)C(C)C)[C@H](C)OCc1ccccc1 ; CCC[C@@H](CCCCC(NC(=O)OC(C)(C)C)C(=O)O)[C@@H](O[Si](C(C)C)(C(C)C)C(C)C)[C@H](C)O]

The title compound was prepared from (7S,8R,9S)-benzyl 9-(benzyloxy)-2-((tert-butoxycarbonyl)amino)-7-propyl-8-((triisopropylsilyl)oxy)decanoate according to the methodology outlined in Example 4, Step 3B and was isolated as a hard foam in 95%: ¹H NMR (400 MHz, CDCl₃) δ 5.02 (d, J=8.1 Hz, 1H), 4.29 (d, J=5.2 Hz, 1H), 3.90 (qd, J=6.4, 3.6 Hz, 1H), 3.80 (t, J=3.1 Hz, 1H), 1.86 (s, 1H), 1.65 (dt, J=19.1, 9.7 Hz, 1H), 1.50 (s, 3H), 1.45 (s, 9H), 1.42-1.22 (m, 8H), 1.18 (d, J=6.4 Hz, 3H), 1.09 (s, 21H), 0.89 (t, J=7.1 Hz, 3H); 13C NMR (101 MHz, CDCl₃) δ 176.92, 155.65, 80.18, 77.97, 70.30, 53.43, 41.56, 33.05, 32.34, 30.71, 28.31, 27.83, 25.79, 21.38, 18.60, 18.30, 14.57, 13.05; HRMS-ESI (m/z) [M+Na]⁺ calcd for C₂₇H₅₅NNaO₆Si, 540.3691; found, 540.3718.

### Example 5, Step 6: Preparation of tert-butyl ((3S,8S,9R,10S)-10-methyl-2-oxo-8-propyl-9-((triisopropylsilyl)oxy)oxecan-3-yl)carbamate (Cmpd 231)

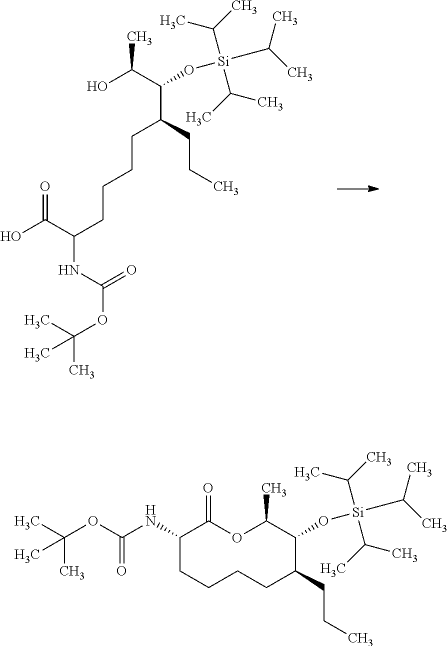

The title compound was prepared from (7S,8R,9S)-2-((tert-butoxycarbonyl)amino)-9-hydroxy-7-propyl-8-((triisopropylsilyl)oxy)decanoic acid according to the methodology outlined in Example 1, Step 5 and was isolated as a colorless oil in 14% yield: ¹H NMR (400 MHz, CDCl₃) δ 5.27 (d, J=6.5 Hz, 1H), 5.03 (t, J=6.6 Hz, 1H), 4.34 (s, 1H), 3.66 (t, J=6.5 Hz, 1H), 2.13-2.00 (m, 1H), 1.90 (dd, J=13.5, 6.4 Hz, 1H), 1.58 (ddd, J=12.4, 9.4, 5.8 Hz, 2H), 1.48 (s, 5H), 1.45 (s, 11H), 1.40 (dd, J=10.1, 2.7 Hz, 4H), 1.34 (d, J=6.6 Hz, 3H), 1.32-1.15 (m, 4H), 1.18-1.05 (m, 22H), 0.88 (t, J=7.1 Hz, 3H); ¹³C NMR (101 MHz, CDCl₃) δ 172.39, 155.23, 79.66, 79.56, 76.80, 53.26, 43.88, 35.58, 28.33, 27.69, 24.16, 22.16, 21.50, 18.84, 18.29, 18.20, 18.18, 14.29, 13.43; HRMS-ESI (m/z) [M+Na]⁺ calcd for C₂₇H₅₃NNaO₅Si, 522.3585; found, 522.3596.

### Example 5, Step 7: Preparation of tert-butyl ((3S,8S,9R,10S)-9-hydroxy-10-methyl-2-oxo-8-propyloxecan-3-yl)carbamate (Cmpd 230)

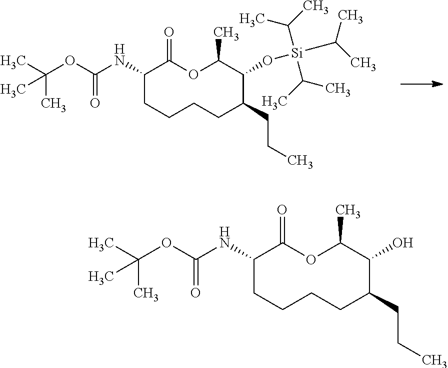

To a solution of tert-butyl ((3S,8S,9R,10S)-10-methyl-2-oxo-8-propyl-9-((triisopropylsilyl)oxy)-oxecan-3-yl)carbamate (749 mg, 1.50 mmol) in THF (15 mL) was added TBAF (1 M in THF, 2.2 mL, 2.2 mmol) and the resulting yellow solution was stirred for 3 h at room temperature. The reaction mixture was poured into brine (10 mL) and H₂O (10 mL), extracted with EtOAc (3×20 mL), and the extracts were combined, dried over MgSO₄, filtered, and concentrated to provide an oil which was purified by column chromatography (SiO₂, 2→20% acetone in hexanes) to provide the title compound (431 mg, 84%) as a hard, white foam: ¹H NMR (400 MHz, CDCl₃) δ 5.28 (s, 1H), 4.82 (dq, J=9.1, 6.2 Hz, 1H), 4.35 (s, 1H), 3.41-3.21 (m, 1H), 2.09 (s, 1H), 1.90 (dt, J=15.0, 5.3 Hz, 1H), 1.60 (dd, J=10.9, 6.2 Hz, 3H), 1.50 (d, J=20.9 Hz, 4H), 1.45 (s, 9H), 1.38 (d, J=6.2 Hz, 3H), 1.33-1.14 (m, 4H), 1.07 (d, J=14.1 Hz, 1H), 0.90 (t, J=7.0 Hz, 3H); ¹³C NMR (101 MHz, CDCl₃) δ 173.11, 155.20, 79.74, 75.73, 52.56, 43.95, 34.95, 28.35, 27.00, 26.76, 24.89, 21.77, 19.47, 18.75, 14.48; ESIMS m/z 366.3 ([M+Na]⁺).

### Example 6, Step 1: Preparation of (2S,3R,4S)-2-methyl-3,4-dihydro-2H-pyran-3,4-diol

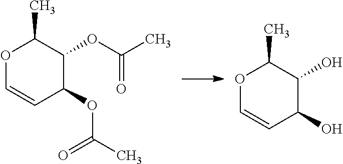

To a magnetically stirred solution of (2S,3S,4S)-2-methyl-3,4-dihydro-2H-pyran-3,4-diyl diacetate (32.16 g, 150 mmol) in MeOH (150 mL) was added K₂CO₃ (2.075 g, 15.01 mmol) and the resulting solution was stirred at 20° C. for 16 h. The reaction mixture was filtered through a 6×2 centimeter (cm) plug of SiO₂, rinsing with EtOAc (500 mL), and the solvent was removed in vacuo to provide the title compound (19.62 g, 100%) as a yellow solid: ¹H NMR (400 MHz, CDCl₃) δ 6.30 (dd, J=6.0, 1.7 Hz, 1H), 4.70 (dd, J=6.0, 2.0 Hz, 1H), 4.20 (dt, J=7.5, 1.9 Hz, 1H), 3.85 (dq, J=9.8, 6.3 Hz, 1H), 3.40 (dd, J=9.9, 7.4 Hz, 1H), 1.38 (d, J=6.3 Hz, 3H).

### Example 6, Step 2: Preparation of (2S,3S,4S)-3,4-bis((4-methoxybenzyl)oxy)-2-methyl-3,4-dihydro-2H-pyran

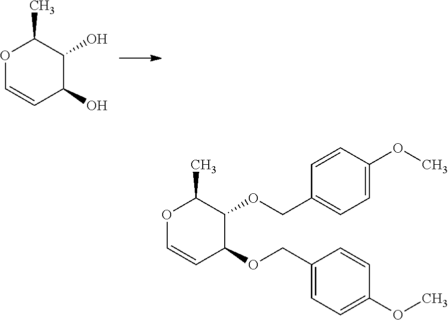

To a suspension of hexane-washed NaH (2.58 g, 64.5 mmol; 60% dispersion in mineral oil) in DMF (38 mL) was added a solution of (2S,3R,4S)-2-methyl-3,4-dihydro-2H-pyran-3,4-diol (3.00 g, 23.0 mmol) in DMF (8 mL) dropwise over a 30 min period at 0° C. The reaction mixture was stirred at 0° C. for an additional 30 min and treated with 1-(bromomethyl)-4-methoxybenzene (11.59 g, 57.6 mmol) dropwise over a 20 min period at 0° C. The resulting thick mixture was warmed to room temperature and stirred for 1 h, recooled to 0° C., and treated with diethylamine (4.77 mL, 46.1 mmol). The reaction mixture was warmed to room temperature, stirred for 1 h, and was then quenched via the addition of sat'd aq NH₄Cl (2 mL). The mixture was partitioned between Et₂O (100 mL) and H₂O (100 mL) and the phases were separated. The aq phase was extracted with Et₂O (2×50 mL), and the combined organics were washed with H₂O (100 mL) and brine (100 mL), dried over calcium chloride (CaCl₂), filtered, and concentrated to provide a yellow oil which was purified by column chromatography (SiO₂, 0→25% EtOAc in hexanes) to provide the title compound (8.14 g, 95%) as a clear, colorless oil: IR (Thin Film) 2934.35, 2900.28, 2835.31, 1611.79, 1511.95, 1243.91 cm⁻¹; ¹H NMR (400 MHz, DMSO-d₆) δ 7.31-7.18 (m, 5H), 6.95-6.85 (m, 5H), 6.38 (dd, J=6.0, 1.3 Hz, 1H), 4.90 (dd, J=6.1, 2.6 Hz, 1H), 4.67 (d, J=11.1 Hz, 1H), 4.60-4.51 (m, 2H), 4.44 (d, J=11.4 Hz, 1H), 4.08-4.00 (m, 1H), 3.89 (dq, J=8.4, 6.5 Hz, 1H), 3.74 (s, 3H), 3.74 (s, 3H), 3.38 (dd, J=8.4, 6.2 Hz, 1H), 1.26 (d, J=6.4 Hz, 3H); ¹³C NMR (101 MHz, DMSO-d₆) δ 158.70, 158.64, 144.07, 130.47, 130.38, 129.38, 129.28, 113.58, 113.53, 100.32, 78.33, 74.72, 73.08, 72.35, 69.04, 55.00 (2C), 17.11.

### Example 6, Step 3: Preparation of (3R,4S,5S)-3,4-bis((4-methoxybenzyl)oxy)-5-methyltetra-hydrofuran-2-ol

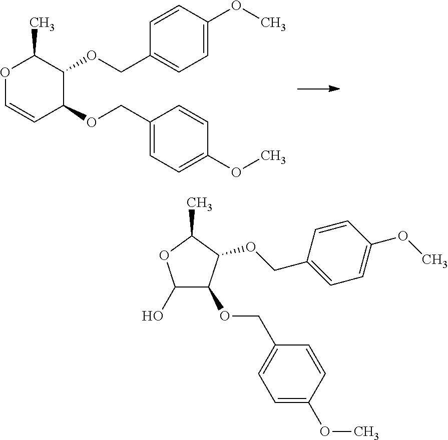

To a solution of (2S,3S,4S)-3,4-bis((4-methoxybenzyl)oxy)-2-methyl-3,4-dihydro-2H-pyran (1.00 g, 2.70 mmol) and NaHCO₃ (0.023 g, 0.27 mmol) in CH₂Cl₂ (8 mL) and MeOH (0.82 μL) was added Sudan III (50 uL of 1% in CH₂Cl₂) and the mixture was cooled to −78° C. Ozone was bubbled through the solution until the light pink/red color dissipated. The solution was purged with oxygen for 10 min, treated with (CH₃)₂S (397 μL, 5.40 mmol), warmed to room temperature, stirred for 1 h, and concentrated in vacuo. The crude residue was dissolved in a mixture of THF (8 mL) and H₂O (4 mL), treated with LiOH—H₂O (340 mg, 8.10 mmol), and the biphasic mixture was stirred vigorously for 2 h. The reaction mixture was poured into 1 N HCl (10 mL) and the phases were separated. The aq phase was extracted with EtOAc (3×15 mL) and the combined organics were washed with brine (20 mL), dried over Na₂SO₄, filtered and concentrated. The crude residue was purified by column chromatography (SiO₂, 0→50% EtOAc in hexanes) to provide the title compound (846 mg, 84%) as a pale-yellow oil: IR (Thin Film) 3410.81, 2932.59, 2835.96, 1611.49, 1512.32, 1245.16 cm⁻¹; ¹H NMR (400 MHz, CDCl₃) δ (major epimer) 7.31-7.24 (m, 2H), 7.24-7.18 (m, 2H), 6.88 (ddd, J=8.7, 6.1, 2.7 Hz, 4H), 5.35 (d, J=7.1 Hz, 1H), 4.59-4.52 (m, 1H), 4.52-4.42 (m, 3H), 4.32 (qd, J=6.5, 4.4 Hz, 1H), 3.97-3.88 (m, 1H), 3.86-3.77 (m, 6H), 3.72-3.61 (m, 1H), 3.14 (d, J=7.2 Hz, 1H), 1.29 (d, J=6.5 Hz, 3H); ¹³C NMR (101 MHz, CDCl₃) δ (major epimer) 159.42, 159.41, 129.77, 129.49, 129.47, 129.33, 113.90, 113.89, 100.92, 95.89, 86.95, 86.86, 78.74, 71.81, 71.61, 55.30, 19.39.

### Example 6 Step 4: Preparation of (2S,3S,4S)-2,3-bis((4-methoxybenzyl)oxy)pentane-1,4-diol

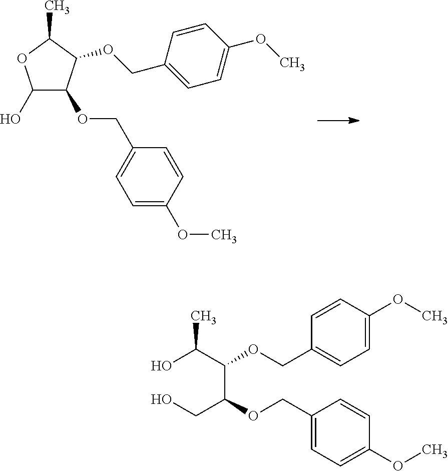 [SMILES: COc1ccc(CO[C@H]2[C@H](C)OC(O)[C@@H]2OCc2ccc(OC)cc2)cc1 ; COc1ccc(CO[C@@H]([C@H](C)O)[C@H](CO)OCc2ccc(OC)cc2)cc1]

To a solution of (3R,4S,5S)-3,4-bis((4-methoxybenzyl)oxy)-5-methyltetrahydrofuran-2-ol (800 mg, 2.14 mmol) in EtOH (8.5 mL) was added NaBH₄ (162 mg, 4.27 mmol), and the reaction mixture was stirred at ambient temperature for 1 h, quenched by the dropwise addition of sat'd aq NH₄Cl (1 mL), and partitioned between EtOAc (10 mL) and H₂O (10 mL). The phases were separated and the aq phase was extracted with EtOAc (2×10 mL). The combined organics were washed with brine (20 mL), dried over Na₂SO₄, filtered, and concentrated to provide the title compound (856 mg, 98%) as a clear, colorless oil: IR (Thin Film) 3413.12, 2933.21, 2836.01, 1611.49, 1512.33, 1244.88 cm⁻¹; ¹H NMR (400 MHz, CDCl₃) δ 7.26-7.22 (m, 4H), 6.91-6.85 (m, 4H), 4.60-4.54 (m, 4H), 3.98 (td, J=6.4, 4.2 Hz, 1H), 3.89-3.76 (m, 7H), 3.72 (ddt, J=7.2, 4.4, 2.2 Hz, 2H), 3.41 (dd, J=6.4, 4.0 Hz, 1H), 3.02 (d, J=4.6 Hz, 1H), 2.32-2.21 (m, 1H), 1.21 (d, J=6.4 Hz, 3H); ¹³C NMR (101 MHz, CDCl₃) δ 159.54, 159.46, 129.85, 129.80, 129.77, 129.62, 113.99, 113.92, 81.01, 78.81, 72.99, 72.21, 67.50, 61.41, 55.28, 19.73, 14.20.

### Example 6 Step 5: Preparation of (2S,3S,4S)-3,4-bis((4-methoxybenzyl)oxy)-5-((3-methylbut-2-en-1-yl)oxy)pentan-2-ol

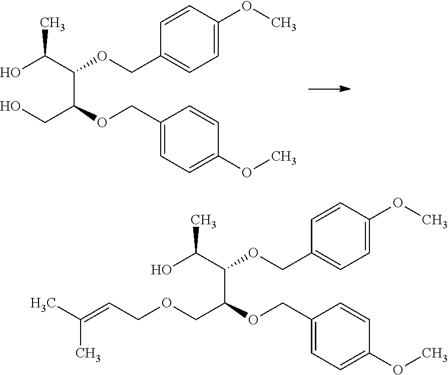

To a flask containing neat (2S,3S,4S)-2,3-bis((4-methoxybenzyl)oxy)pentane-1,4-diol (10.25 g, 27.2 mmol) was added a solution of NaOH (13.07 g, 327 mmol) in H₂O (109 mL), and the reaction mixture was treated with N,N-dibutyl-N-methylbutan-1-aminium chloride (1.284 g, 5.45 mmol) and 1-bromo-3-methylbut-2-ene (5.07 g, 34.0 mmol), and the heterogeneous mixture was stirred vigorously for 2 d at room temperature. The mixture was partitioned between EtOAC (100 mL) and H₂O (50 mL) and the phases were separated. The aq phase was extracted with EtOAc (2×100 mL), and the combined organics were washed with H₂O (100 mL) and brine (100 mL), dried over Na₂SO₄, filtered and concentrated. The crude residue was purified by column chromatography (SiO₂, 0→25% acetone in hexanes) to provide the title compound (8.837 g, 60%) as a pale-yellow oil: IR (Thin Film) 3465.03, 2909.95, 2835.93, 1611.77, 1512.49, 1245.29 cm⁻¹; ¹H NMR (400 MHz, CDCl₃) δ 7.31-7.25 (m, 2H), 7.25-7.18 (m, 2H), 6.91-6.81 (m, 4H), 5.33 (dddq, J=8.3, 5.7, 2.8, 1.4 Hz, 1H), 4.68 (d, J=11.4 Hz, 1H), 4.58-4.45 (m, 3H), 3.97 (dq, J=7.1, 1.0 Hz, 2H), 3.94-3.78 (m, 8H), 3.69-3.59 (m, 2H), 3.33 (dd, J=6.7, 4.0 Hz, 1H), 3.10 (d, J=4.8 Hz, 1H), 1.75 (q, J=1.1 Hz, 3H), 1.69-1.64 (m, 3H), 1.17 (d, J=6.3 Hz, 3H); ¹³C NMR (101 MHz, CDCl₃) δ 159.35, 159.30, 137.02, 130.31, 130.13, 129.80, 129.68, 120.97, 113.81, 113.78, 81.06, 77.98, 72.93, 72.37, 69.51, 67.85, 67.53, 55.27, 25.81, 19.91, 18.08.

### Example 6, Step 6: Preparation of 2-(((2S,3S,4S)-4-hydroxy-2,3-bis((4-methoxybenzyl)oxy)-pentyl)oxy)acetaldehyde

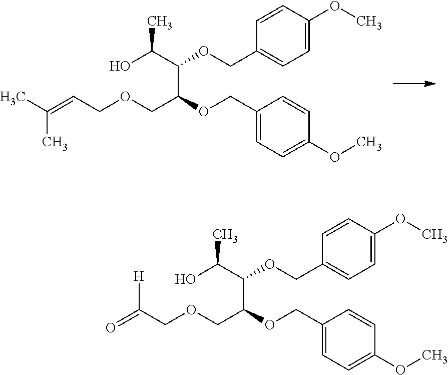 [SMILES: COc1ccc(CO[C@@H]([C@H](C)O)[C@H](COCC=C(C)C)OCc2ccc(OC)cc2)cc1 ; COc1ccc(CO[C@@H]([C@H](C)O)[C@H](COCC=O)OCc2ccc(OC)cc2)cc1]

To a solution of (2S,3S,4S)-3,4-bis((4-methoxybenzyl)oxy)-5-((3-methylbut-2-en-1-yl)oxy)pentan-2-ol (8.83 g, 19.86 mmol) in CH₂Cl₂ (60 mL) were added MeOH (6.0 mL), NaHCO₃ (167 mg, 1.99 mmol), and Sudan III (0.2 mg, 0.6 μmol). The mixture was cooled to −78° C. and O₃ was bubbled through the solution until the light pink/red color dissipated. The solution was purged with oxygen for 10 min, treated with (CH₃)₂S (2.92 μL, 39.7 mmol), warmed to room temperature, stirred for 1 h, and concentrated in vacuo. The reaction mixture was then concentrated and the residue purified by column chromatography (SiO₂, 0→60% EtOAc in hexanes) to provide the title compound (9.779 g, 100%) as a clear oil: ESI-MS m/z 441.2 ([M+Na]⁺).

### Example 6, Step 7: Preparation of (Z)-methyl 2-((tert-butoxycarbonyl)amino)-4-(((2S,3S,4S)-4-hydroxy-2,3-bis((4-methoxybenzyl)oxy)pentyl)oxy)but-2-enoate

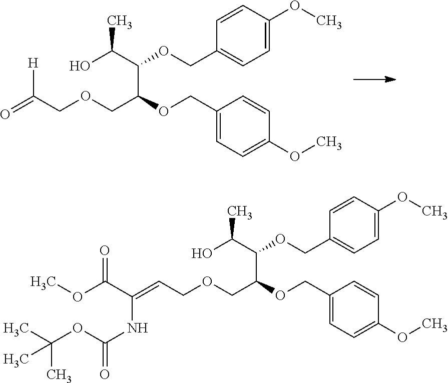

To a solution of 2-(((2S,3S,4S)-4-hydroxy-2,3-bis((4-methoxybenzyl)oxy)pentyl)oxy)-acetaldehyde (8.3 g, 19.8 mmol) in CH₂Cl₂ (198 mL) were added methyl 2-((tert-butoxycarbonyl)amino)-2-(dimethoxyphosphoryl)acetate (6.48 g, 21.8 mmol) followed by the dropwise addition of DBU (3.01 mL, 21.8 mmol). The reaction mixture was stirred overnight, quenched with H₂O (100 mL), and the phases were separated. The aq phase was extracted with CH₂Cl₂ (2×100 mL) and the combined organics were dried by passing through a phase separator cartridge and concentrated. The crude residue was purified by column chromatography (SiO₂, 0440% acetone in hexanes) to provide the title compound (11.7 g, 100%) as a clear oil: IR (Thin Film) 3408.38, 2933.25, 2837.28, 1715.78, 1512.55, 1243.84 cm⁻¹; ¹H NMR (400 MHz, CDCl₃) δ 7.27 (dd, J=6.8, 1.9 Hz, 2H), 7.25-7.19 (m, 2H), 6.92-6.82 (m, 4H), 6.54-6.44 (m, 2H), 4.67 (d, J=11.5 Hz, 1H), 4.59-4.50 (m, 3H), 4.15 (dd, J=5.8, 2.8 Hz, 2H), 3.94-3.85 (m, 1H), 3.85-3.78 (m, 10H), 3.72-3.63 (m, 2H), 3.33 (dd, J=6.7, 4.0 Hz, 1H), 3.00 (d, J=4.7 Hz, 1H), 1.46 (s, 9H), 1.17 (d, J=6.2 Hz, 3H); HRMS-ESI (m/z) [M+Na]⁺ calcd for C₃₁H₄₃NO₁₀Na, 613.2813; found, 613.2786.

### Example 6, Step 8: Preparation of (S)-methyl 2-((tert-butoxycarbonyl)amino)-4-(((2S,3S,4S)-4-hydroxy-2,3-bis((4-methoxybenzyl)oxy)pentyl)oxy)butanoate

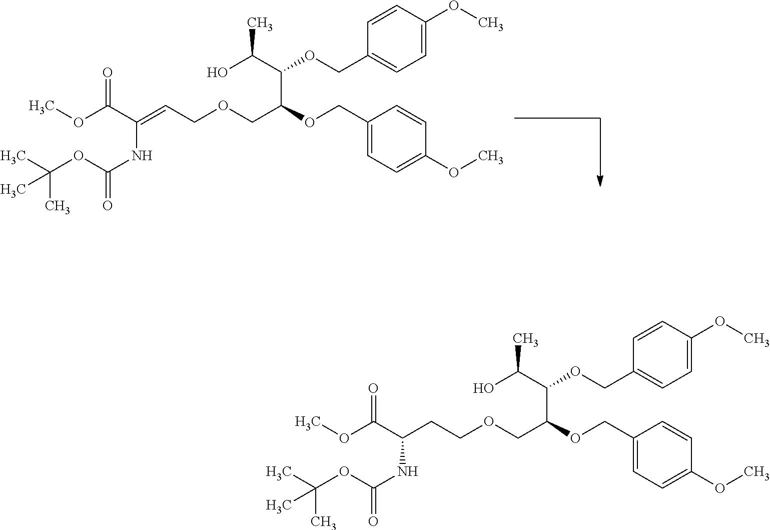

To a high pressure reactor were added (Z)-methyl 2-((tert-butoxycarbonyl)amino)-4-(((2S,3S,4S)-4-hydroxy-2,3-bis((4-methoxybenzyl)oxy)pentyl)oxy)but-2-enoate (11.7 g, 19.8 mmol) and MeOH (99 mL), and the resulting solution was sparged with N₂ for 45 min and treated with S,S-DuPhos-Rh (0.287 g, 0.397 mmol). The reactor was sealed, flushed with H₂, pressurized to 200 psi, and the mixture was stirred at room temperature for 60 h. The reactor was vented, the mixture was concentrated, and the residue purified by column chromatography (SiO₂, 0→100% EtOAc in hexanes) to provide the title compound (10.37 g, 79%) as clear, pale-yellow oil: IR (Thin Film) 3405.20, 2933.00, 1744.57, 1711.35, 1611.85, 1512.56, 1245.69 cm⁻¹; ¹H NMR (400 MHz, CDCl₃) δ 7.31-7.27 (m, 2H), 7.22 (d, J=8.6 Hz, 2H), 6.90-6.84 (m, 4H), 5.52 (d, J=8.2 Hz, 1H), 4.67 (d, J=11.5 Hz, 1H), 4.58-4.49 (m, 3H), 4.47-4.36 (m, 1H), 3.93-3.84 (m, 1H), 3.83-3.75 (m, 7H), 3.72 (s, 3H), 3.64-3.59 (m, 2H), 3.58-3.41 (m, 2H), 3.37-3.27 (m, 1H), 3.02-2.95 (m, 1H), 2.16-2.04 (m, 1H), 2.03-1.91 (m, 1H), 1.42 (s, 9H), 1.18 (d, J=6.3 Hz, 3H): HRMS-ESI (m/z) [M+Na]⁺ calcd for C₃₁H₄₅NO₁₀Na, 614.2936; found, 614.2920.

### Example 6, Step 9: Preparation of (S)-2-((tert-butoxycarbonyl)amino)-4-(((2S,3S,4S)-4-hydroxy-2,3-bis((4-methoxybenzyl)oxy)pentyl)oxy)butanoic acid

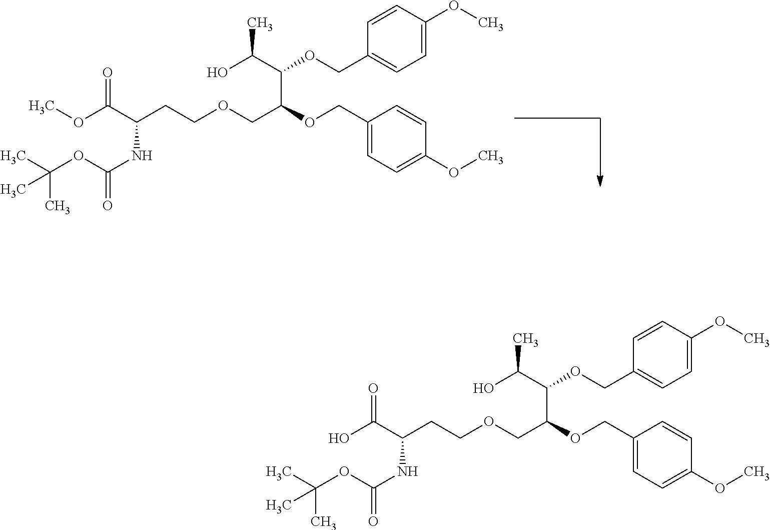

The title compound was prepared from (S)-methyl 2-((tert-butoxycarbonyl)amino)-4-(((2S,3S,4S)-4-hydroxy-2,3-bis((4-methoxybenzyl)oxy)pentyl)oxy)butanoate according to the methodology outlined in Example 1, Step 4 and was used directly in the next reaction.

### Example 6, Step 10: Preparation of tert-butyl ((3S,8S,9S,10S)-8,9-bis((4-methoxybenzyl)oxy)-10-methyl-2-oxo-1,6-dioxecan-3-yl)carbamate (Cmpd 225)

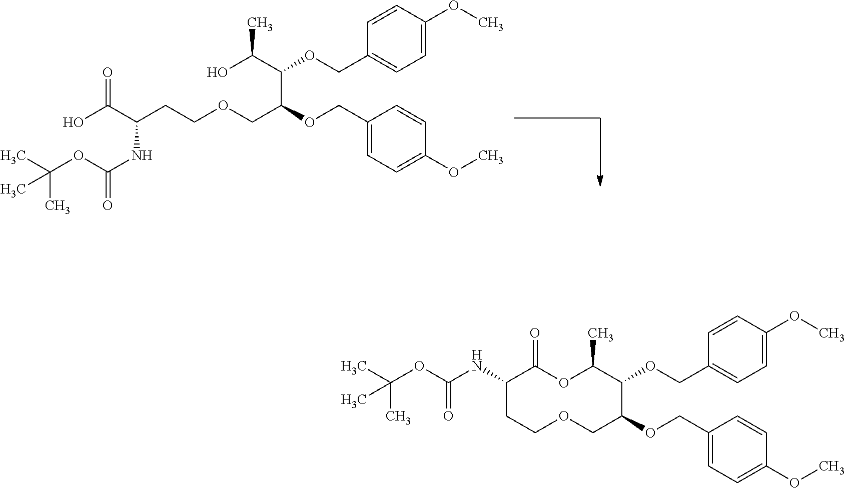 [SMILES: COc1ccc(CO[C@@H]([C@H](C)O)[C@H](COCC[C@H](NC(=O)OC(C)(C)C)C(=O)O)OCc2ccc(OC)cc2)cc1 ; COc1ccc(CO[C@H]2[C@H](C)OC(=O)[C@@H](NC(=O)OC(C)(C)C)CCOC[C@@H]2OCc2ccc(OC)cc2)cc1]

The title compound was prepared from (S)-2-((tert-butoxycarbonyl)amino)-4-(((2S,3S,4S)-4-hydroxy-2,3-bis((4-methoxybenzyl)oxy)pentyl)oxy)butanoic acid according to the methodology outlined in Example 1, Step 5 and was isolated as a colorless oil in 34% yield: ¹H NMR (400 MHz, CDCl₃) δ 7.25-7.18 (m, 4H), 6.89-6.82 (m, 4H), 5.49 (d, J=6.2 Hz, 1H), 5.03-4.94 (m, 1H), 4.84 (d, J=10.3 Hz, 1H), 4.58 (s, 2H), 4.53 (d, J=10.4 Hz, 1H), 4.30 (s, 1H), 3.88-3.77 (m, 7H), 3.61-3.31 (m, 5H), 2.56-2.41 (m, 1H), 1.79 (d, J=15.1 Hz, 1H), 1.44 (s, 9H), 1.35 (d, J=6.3 Hz, 3H); ¹³C NMR (101 MHz, CDCl₃) δ 172.09, 159.26, 159.25, 155.13, 130.41, 130.30, 129.63, 129.47, 113.82, 113.79, 83.60, 82.34, 79.80, 75.53, 72.81, 71.55, 69.01, 65.13, 55.28, 55.27, 50.99, 29.21, 28.34, 18.52; HRMS-ESI (m/z) [M+Na]⁺ calcd for C₃₀H₄₁NO₉Na, 582.2674; found, 582.2651.

### Example 6, Step 11: Preparation of tert-butyl ((3S,8S,9R,10S)-8,9-dihydroxy-10-methyl-2-oxo-1,6-dioxecan-3-yl)carbamate (Cmpd 246)

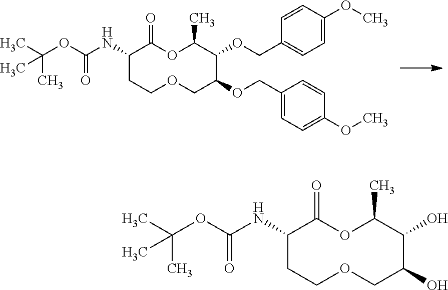

To a solution of tert-butyl ((3S,8S,9S,10S)-8,9-bis((4-methoxybenzyl)oxy)-10-methyl-2-oxo-1,6-dioxecan-3-yl)carbamate (2.54 g, 4.54 mmol) in CH₃CN (41 mL) at 0° C. were added H₂O (4.1 mL) and CAN (12.44 g, 22.69 mmol), and the mixture was warmed to room temperature, stirred for 1 h, and treated with Na₂SO₄ (30 g). The solids were removed by filtration through a pad of Celite® and the filter cake was washed with CH₂Cl₂ (3×100 mL). The organics were washed with NaHCO₃ (50 mL), and the aq wash was back extracted with CH₂Cl₂ (3×50 mL). The combined organics were washed with H₂O (200 mL) and brine (200 mL), dried over Na₂SO₄, filtered, and concentrated. The resulting residue was purified by column chromatography (SiO₂, 0→100% acetone in hexanes) to provide the title compound (1.002 g, 69%) as a white solid: IR (Thin Film) 3537.78, 3483.85, 3350.99, 2931.70, 1728.70, 1681.83, 1520.26, 1364.51, 1159.49 cm⁻¹; ¹H NMR (400 MHz, CDCl₃) δ 5.58 (s, 1H), 5.04-4.94 (m, 1H), 4.36-4.26 (m, 1H), 3.85 (t, J=11.5 Hz, 1H), 3.60-3.45 (m, 4H), 3.45-3.36 (m, 1H), 3.36-3.26 (m, 1H), 2.52-2.39 (m, 1H), 1.84-1.73 (m, 1H), 1.44 (s, 9H), 1.38 (d, J=6.3 Hz, 3H), 1.34 (d, J=1.1 Hz, 1H); HRMS-ESI (m/z) [M+Na]⁺ calcd for C₁₄H₂₅NO₇Na, 342.1523; found, 342.1528.

### Example 7, Step 1: Preparation of (2S,3S,4S)-3,4-bis(benzyloxy)hept-6-en-2-ol

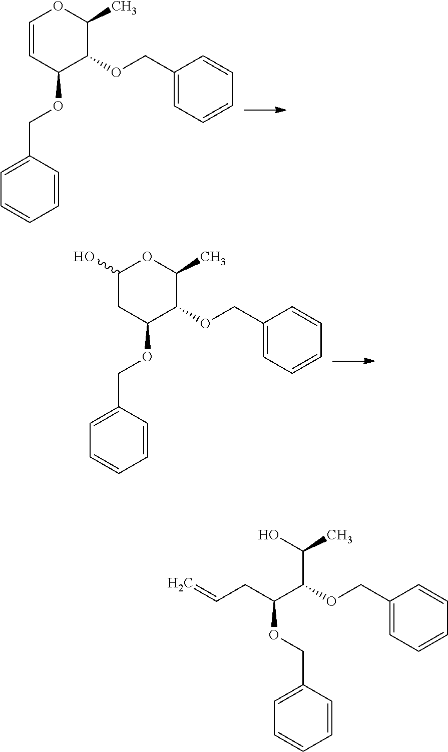 [SMILES: C[C@@H]1OC=C[C@H](OCc2ccccc2)[C@H]1OCc1ccccc1 ; C[C@@H]1OC(O)C[C@H](OCc2ccccc2)[C@H]1OCc1ccccc1 ; C=CC[C@H](OCc1ccccc1)[C@@H](OCc1ccccc1)[C@H](C)O]

To a solution of (2S,3S,4S)-3,4-bis(benzyloxy)-2-methyl-3,4-dihydro-2H-pyran (30 g, 90 mmol) in CH₃CN (899 mL) were added LiBr (23.42 g, 270 mmol), H₂O (16 mL, 899 mmol), and Dowex® 50WX4 (200 mesh, 2% by mass relative to SM; 600 mg, 1.80 mmol), in that order, and the mixture was stirred at 20° C. for 30 min and treated with NEt₃ (37.6 mL, 270 mmol). The mixture was filtered and the volume of the filtrate was reduced by about 75%. The concentrated solution was diluted with EtOAc (200 mL), washed with 1 N HCl (100 mL) and brine (100 mL), and the combined aq washes were extracted with EtOAc (30 mL). The organic extracts were combined, dried over Na₂SO₄, filtered, and concentrated to provide the intermediate alcohol, (4S,5S,6S)-4,5-bis(benzyloxy)-6-methyltetrahydro-2H-pyran-2-ol (29.25 g, 99%), as a yellow solid, which was immediately used in the next step. To a suspension of methyltriphenylphosphonium bromide (39.2 g, 110 mmol) in THF (400 mL) at 0° C. was added n-BuLi (49.3 mL, 123 mmol) dropwise over a 25 min period, and the resulting solution was stirred for 30 min at 0° C. The resulting dark orange/red reaction mixture was cooled to −78° C. and treated with a solution of the freshly prepared alcohol, (4S,5S,6S)-4,5-bis(benzyloxy)-6-methyltetrahydro-2H-pyran-2-ol (15 g, 45.7 mmol), in THF (50 mL), rinsing the flask and cannula with THF (20 mL). The resulting bright-yellow, heterogeneous mixture was allowed to slowly warm to ambient temperature overnight. The reaction mixture was quenched with H₂O (150 mL), extracted with Et₂O (300 mL), and the phases separated. The organic phase was sequentially washed with sat'd aq NH₄Cl (800 mL) and brine (800 mL), dried over MgSO₄, filtered, and concentrated. The resulting oil was purified by column chromatography (SiO₂, 1→50% EtOAc in hexanes) to provide the title compound (13.4 g, 76%) as a yellow liquid: ¹H NMR (400 MHz, CDCl₃) δ 7.38-7.27 (m, 10H), 5.82 (ddt, J=17.2, 10.1, 7.0 Hz, 1H), 5.14-5.03 (m, 2H), 4.64-4.59 (m, 3H), 4.57 (d, J=11.4 Hz, 1H), 4.05-3.95 (m, 1H), 3.70 (dt, J=7.6, 4.7 Hz, 1H), 3.36 (dd, J=6.5, 4.4 Hz, 1H), 3.04 (d, J=4.7 Hz, 1H), 2.52 (dddt, J=8.1, 6.2, 4.8, 1.3 Hz, 1H), 2.44-2.34 (m, 1H), 1.22-1.19 (m, 3H); ¹³C NMR (101 MHz, CDCl₃) δ 138.25, 137.72, 134.94, 128.48, 128.44, 128.18, 127.96, 127.95, 127.81, 117.35, 81.60, 79.47, 73.50, 72.52, 67.25, 34.35, 19.65; ESI-MS m/z 349.3 ([M+Na]⁺).

### Example 7, Step 2: Preparation of (2S,3S,4S)-3,4-bis(benzyloxy)hept-6-en-2-yl acetate

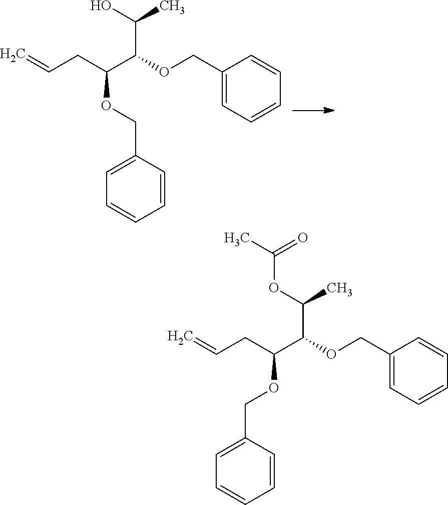 [SMILES: C=CC[C@H](OCc1ccccc1)[C@@H](OCc1ccccc1)[C@H](C)O ; C=CC[C@H](OCc1ccccc1)[C@@H](OCc1ccccc1)[C@H](C)OC(C)=O]

To a solution of (2S,3S,4S)-3,4-bis(benzyloxy)hept-6-en-2-ol (3.0 g, 9.2 mmol) in CH₂Cl₂ (37 mL) at 0° C. were added NEt₃ (1.60 mL, 11.5 mmol), DMAP (0.112 g, 0.919 mmol), and acetic anhydride (0.95 mL, 10.1 mmol), in that order, and the mixture was stirred at 0° C. for 1 h, diluted with CH₂Cl₂ (20 mL), and quenched with sat'd aq NH₄Cl (50 mL). The phases were separated and the organic phase was washed with sat'd aq NaHCO₃ (50 mL) and brine (50 mL), dried over Na₂SO₄, filtered, and concentrated. The resulting oil was purified by column chromatography (SiO₂, 0→30% EtOAc in hexanes) to provide the title compound (3.03 g, 89%) as a clear, pale-yellow oil: ¹H NMR (400 MHz, CDCl₃) δ 7.40-7.27 (m, 10H), 5.82 (ddt, J=17.2, 10.2, 7.0 Hz, 1H), 5.15-5.04 (m, 3H), 4.73 (d, J=11.5 Hz, 1H), 4.67 (d, J=11.5 Hz, 1H), 4.59 (ABq, J=12.0, 1.4 Hz, 2H), 3.60 (dd, J=5.6, 3.4 Hz, 1H), 3.52 (dt, J=6.3, 5.5 Hz, 1H), 2.48-2.39 (m, 1H), 2.39-2.30 (m, 1H), 1.98 (s, 3H), 1.32 (d, J=6.5 Hz, 3H); ¹³C NMR (101 MHz, CDCl₃) δ 170.23, 138.52, 138.38, 134.49, 128.28, 128.01, 127.98, 127.60, 127.58, 117.53, 81.88, 79.43, 74.38, 72.79, 71.31, 35.38, 21.34, 15.22; HRMS-ESI (m/z) [M+H]⁺ calcd for C₂₃H₂₉O₄, 369.2066; found, 369.2058.

### Example 7, Step 3: Preparation of (7S,8S,9S, Z)-methyl 9-acetoxy-7,8-bis(benzyloxy)-2-((tert-butoxycarbonyl)amino)ec-2-enoate

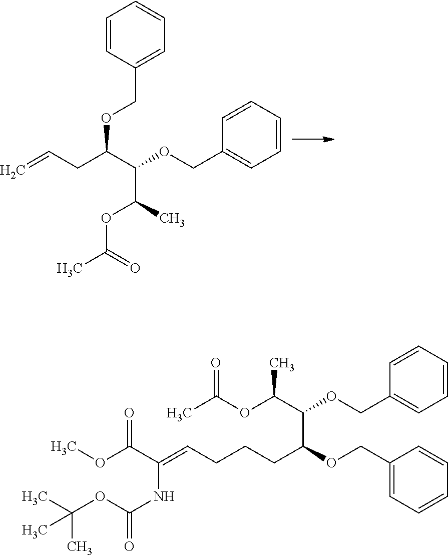 [SMILES: C=CC[C@@H](OCc1ccccc1)[C@H](OCc1ccccc1)[C@@H](C)OC(C)=O ; COC(=O)/C(=C/CCC[C@H](OCc1ccccc1)[C@@H](OCc1ccccc1)[C@H](C)OC(C)=O)NC(=O)OC(C)(C)C]

To a solution of (2S,3S,4S)-3,4-bis(benzyloxy)hept-6-en-2-yl acetate (516 mg, 1.40 mmol) in THF (3.6 mL) was added 9-BBN (3.64 mL, 1.82 mmol, 0.5 M in THF), and the resulting mixture was warmed to and stirred at 50° C. for 2.5 h, treated with additional 9-BBN (0.5 mL), and stirred at 50° C. for an additional 2 h. The reaction mixture was cooled to room temperature and treated with K₃PO₄ (0.934 mL, 2.80 mmol) followed by (Z)-methyl 3-bromo-2-((tert-butoxycarbonyl)-amino)acrylate (392 mg, 1.40 mmol) and PdCl₂(dppf) (51.2 mg, 0.070 mmol), and the resulting mixture was heated to and stirred at 55° C. overnight. The reaction mixture was cooled to room temperature, diluted with Et₂O (25 mL), and quenched by the addition of sat'd aq NaHCO₃ (30 mL). The phases were separated and the aq phase was extracted with Et₂O (2×20 mL). The combined organics were washed with H₂O (25 mL) and brine (3×25 mL), dried over MgSO₄, filtered, concentrated, and the crude oil purified by column chromatography (SiO₂, 0→30% EtOAc in hexanes) to afford the title compound (615 mg, 77%) as a yellow oil: ¹H NMR (400 MHz, CDCl₃) δ 7.38-7.23 (m, 10H), 6.50 (t, J=7.3 Hz, 1H), 6.07 (s, 1H), 5.08 (qd, J=6.5, 3.1 Hz, 1H), 4.73 (d, J=11.6 Hz, 1H), 4.70-4.60 (m, 2H), 4.52 (d, J=11.3 Hz, 1H), 3.76 (d, J=0.9 Hz, 3H), 3.62 (dd, J=6.0, 3.2 Hz, 1H), 3.48-3.38 (m, 1H), 2.17 (q, J=7.0 Hz, 2H), 1.98 (s, 3H), 1.68-1.36 (m, 13H), 1.31 (d, J=6.5 Hz, 3H); ¹³C NMR (101 MHz, CDCl₃) δ 170.30, 165.34, 153.22, 138.49, 138.46, 136.29, 128.31, 128.02, 128.00, 127.65, 127.60, 81.84, 80.48, 79.54, 77.21, 74.30, 73.09, 71.36, 53.42, 52.29, 30.68, 28.21, 24.27, 21.35, 15.11; HRMS-ESI (m/z) [M+Na]⁺ calcd for C₃₂H₄₃NO₈Na, 592.2886; found, 592.2904.

### Example 8A: Preparation of tert-butyl N-tert-butoxycarbonyl-N-[(3S,8S,9R,10S)-8-hydroxy-10-methyl-9-(2-methylallyloxy)-2-oxo-oxecan-3-yl]carbamate (Cmpd 196)

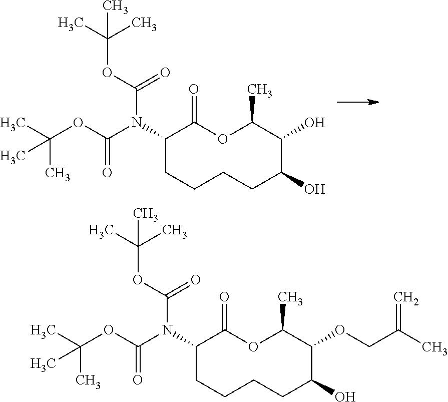 [SMILES: C[C@@H]1OC(=O)[C@@H](N(C(=O)OC(C)(C)C)C(=O)OC(C)(C)C)CCCC[C@H](O)[C@H]1O ; C=C(C)CO[C@H]1[C@H](C)OC(=O)[C@@H](N(C(=O)OC(C)(C)C)C(=O)OC(C)(C)C)CCCC[C@@H]1O]

To a solution of tert-butyl N-tert-butoxycarbonyl-N-[(3S,8S,9R,10S)-8,9-dihydroxy-10-methyl-2-oxo-oxecan-3-yl]carbamate (0.66 g., 1.6 mmol) in degassed THF (10 mL) were added tert-butyl (2-methylallyl) carbonate (0.299 g, 1.74 mmol), DPPF (0.088 g, 0.16 mmol) and Pd₂(dba)₃ (0.079 mmol, 0.072 g), and the red-brown solution was heated to and stirred at 60° C. for 75 min. The solution was cooled to room temperature, concentrated, and the residue purified by column chromatography (SiO₂, 0→10% acetone in hexanes) to provide the title compound (400 mg, 54%) as a light-yellow oil: ¹H NMR (400 MHz, CDCl₃) δ 5.00 (d, J=6.2 Hz, 2H), 4.89 (s, 1H), 4.71 (dq, J=13.7, 6.2 Hz, 1H), 4.17-4.01 (m, 2H), 3.63-3.49 (m, 1H), 3.15 (t, J=8.8 Hz, 1H), 2.62 (s, 1H), 2.08 (dd, J=15.3, 5.3 Hz, 1H), 2.03-1.90 (m, 1H), 1.80 (dd, J=13.6, 5.5 Hz, 1H), 1.75 (s, 3H), 1.57 (d, J=10.7 Hz, 4H), 1.55-1.49 (m, 18H), 1.49-1.45 (m, 1H), 1.42 (d, J=6.2 Hz, 3H); ¹³C NMR (151 MHz, CDCl₃) δ 169.36, 152.93, 141.78, 112.18, 85.69, 82.64, 73.41, 73.09, 58.29, 30.68, 28.45, 27.99, 24.00, 23.93, 19.68, 18.39; ESIMS m/z 494.4 ([M+Na]⁺).

### Example 8B: Preparation of [(2S,3S,4S,9S)-9-[bis(tert-butoxycarbonyl)amino]-3-hydroxy-2-methyl-10-oxo-oxecan-4-yl]benzoate and [(2S,3R,4S,9S)-9-[bis(tert-butoxycarbonyl)-amino]-4-hydroxy-2-methyl-10-oxo-oxecan-3-yl] benzoate (Cmpd 207 and Cmpd 206)

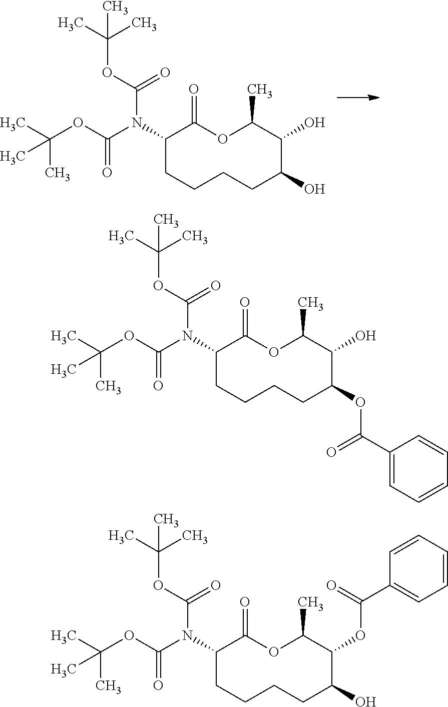

To a solution of tert-butyl N-tert-butoxycarbonyl-N-[(3S,8S,9R,10S)-8,9-dihydroxy-10-methyl-2-oxo-oxecan-3-yl]carbamate (500 mg, 1.20 mmol) in anhydrous pyridine (7.2 mL) at 0° C. were added DMAP (29.3 mg, 0.240 mmol) and benzoyl chloride (167 μL, 1.44 mmol), and the resulting solution was removed from the cold bath and warmed to and stirred at room temperature for 16 h. The reaction mixture was quenched with H₂O (2 mL) and stirred at room temperature for 15 min, partitioned between Et₂O (20 mL) and H₂O (20 mL), and the phases were separated. The aq phase was extracted with Et₂O (2×20 mL) and the combined organic extracts were washed with brine (20 mL), dried over MgSO₄, filtered, and concentrated to a yellow oil which was purified by column chromatography (SiO₂, 0→16% acetone in hexanes) to provide the title compounds:

(2S,3S,4S,9S)-9-((di-tert-butoxycarbonyl)amino)-3-hydroxy-2-methyl-10-oxooxecan-4-yl benzoate (501 mg, 80%) was isolated as a sticky solid: ¹H NMR (400 MHz, CDCl₃) δ 8.63 (d, J=4.2 Hz, 1H), 8.09-7.97 (m, 2H), 7.58 (tt, J=6.9, 1.3 Hz, 1H), 7.52-7.41 (m, 2H), 5.12-4.93 (m, 2H), 4.80 (dq, J=8.8, 6.2 Hz, 1H), 3.84 (t, J=8.7 Hz, 1H), 2.20-2.06 (m, 2H), 2.04-1.95 (m, 1H), 1.89-1.76 (m, 2H), 1.74-1.64 (m, 1H), 1.63-1.56 (m, 1H), 1.52 (s, 18H), 1.51-1.47 (m, 4H); ¹³C NMR (101 MHz, CDCl₃) δ 169.59, 166.64, 152.88, 149.59, 133.23, 130.03, 129.64, 128.46, 82.77, 78.63, 75.25, 73.51, 57.96, 28.55, 28.42, 28.00, 24.31, 22.84, 18.00; ESIMS m/z 520.4 ([M−H]⁻); and

(2S,3R,4S,9S)-9-((di-tert-butoxycarbonyl)amino)-4-hydroxy-2-methyl-10-oxooxecan-3-yl benzoate (20 mg, 3.2% yield) was isolated as a clear, colorless oil: ¹H NMR (400 MHz, CDCl₃) δ 8.12-8.02 (m, 2H), 7.63-7.54 (m, 1H), 7.51-7.43 (m, 2H), 5.18 (t, J=8.6 Hz, 1H), 5.00 (td, J=6.4, 2.5 Hz, 2H), 3.82 (ddd, J=8.4, 5.5, 2.9 Hz, 1H), 2.22-2.06 (m, 2H), 2.02-1.94 (m, 1H), 1.82-1.57 (m, 6H), 1.52 (s, 18H), 1.38 (d, J=6.3 Hz, 3H); ¹³C NMR (151 MHz, CDCl₃) δ 169.58, 166.25, 152.90, 133.45, 129.87, 129.65, 129.50, 128.54, 82.73, 77.95, 72.90, 71.69, 58.23, 34.68, 31.17, 28.73, 28.00, 25.29, 24.28, 22.85, 17.92; ESIMS m/z 520.4 ([M−H]⁻).

### Example 8C: Preparation of tert-butyl ((3S,8S,9S,10S)-10-methyl-2-oxo-8,9-diphenoxyoxecan-3-yl)carbamate, tert-butyl ((3S,8S,9R,10S)-8-hydroxy-10-methyl-2-oxo-9-phenoxyoxecan-3-yl)carbamate, and tert-butyl ((3S,8S,9S,10S)-9-hydroxy-10-methyl-2-oxo-8-phenoxyoxecan-3-yl)carbamate (Cmpd 233, Cmpd 235, and Cmpd 234)

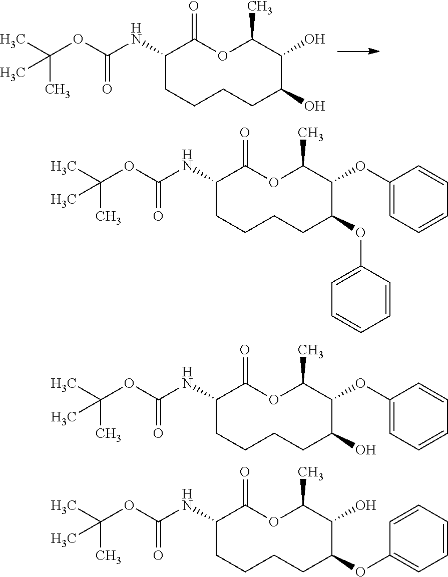

To a solution of tert-butyl ((3S,8S,9R,10S)-8,9-dihydroxy-10-methyl-2-oxooxecan-3-yl)carbamate (1.00 g, 3.15 mmol) in toluene (12.6 mL) was added triphenylbismuth diacetate (3.52 g, 6.30 mmol) and diacetoxycopper (0.114 g, 0.630 mmol), and the mixture was heated to and stirred at 40° C. for 16 h. The reaction mixture was cooled to room temperature, filtered through Celite®, rinsing with toluene (2×15 mL), and the filtrate concentrated. The crude oil was purified by column chromatography (SiO₂, 0→25% acetone in hexanes) to provide the title compounds:

tert-butyl ((3S,8S,9S,10S)-10-methyl-2-oxo-8,9-diphenoxyoxecan-3-yl)carbamate (697 mg, 47%) was isolated as a white solid: IR (Thin Film) 3435, 2976, 1711, 1491, 1167 cm⁻¹; ¹H NMR (400 MHz, CDCl₃) δ 7.24-7.13 (m, 4H), 6.99-6.84 (m, 4H), 6.72-6.66 (m, 2H), 5.32 (d, J=6.7 Hz, 1H), 5.21 (dq, J=9.3, 6.3 Hz, 1H), 4.47 (q, J=10.7, 9.7 Hz, 2H), 4.25 (t, J=7.5 Hz, 1H), 2.23-1.89 (m, 3H), 1.79-1.59 (m, 2H), 1.57-1.50 (m, 2H), 1.46 (s, 9H), 1.39 (d, J=6.3 Hz, 3H), 1.33-1.14 (m, 1H); ¹³C NMR (101 MHz, CDCl₃) δ 172.83, 159.40, 157.84, 155.12, 129.31, 129.23, 121.33, 120.85, 116.27, 115.70, 82.15, 80.15, 72.04, 52.56, 28.35, 28.02, 27.05, 22.67, 21.79, 21.40, 18.45;

tert-butyl ((3S,8S,9R,10S)-8-hydroxy-10-methyl-2-oxo-9-phenoxyoxecan-3-yl)carbamate (592 mg, 48%) was isolated as a white solid: IR (Thin Film) 3436, 2976, 1702, 1491, 1366, 1166 cm⁻¹; ¹H NMR (400 MHz, CDCl₃) δ 7.33-7.27 (m, 2H), 7.02-6.96 (m, 1H), 6.89-6.83 (m, 2H), 5.32 (d, J=6.8 Hz, 1H), 4.98 (dq, J=9.5, 6.2 Hz, 1H), 4.43 (s, 1H), 3.89 (t, J=8.2 Hz, 1H), 3.76 (td, J=8.6, 7.9, 1.1 Hz, 1H), 2.95 (d, J=1.4 Hz, 1H), 2.29-2.16 (m, 1H), 1.96-1.79 (m, 2H), 1.75-1.56 (m, 3H), 1.48-1.41 (m, 12H), 1.39-1.29 (m, 2H); HRMS-ESI (m/z) [M+H]⁺ calcd for C₂₁H₃₁NO₆Na, 416.2044; found, 416.2062; and

tert-butyl ((3S,8S,9S,10S)-9-hydroxy-10-methyl-2-oxo-8-phenoxyoxecan-3-yl)carbamate (58 mg, 4.7%) was isolated as a white solid: IR (Thin Film) 3431, 2976, 1703, 1493, 1366, 1166 cm⁻¹; ¹H NMR (400 MHz, CDCl₃) δ 7.33-7.27 (m, 2H), 7.09-6.93 (m, 3H), 5.35-5.23 (m, 1H), 5.08 (dq, J=9.3, 6.3 Hz, 1H), 4.47-4.37 (m, 1H), 4.16 (t, J=9.0 Hz, 1H), 3.68-3.55 (m, 1H), 2.35 (t, J=1.7 Hz, 1H), 2.19-2.06 (m, 1H), 1.99-1.86 (m, 2H), 1.70-1.50 (m, 3H), 1.49-1.35 (m, 1H), 1.45 (s, 9H), 1.28 (d, J=6.3 Hz, 3H), 1.23-1.12 (m, 1H); HRMS-ESI (m/z) [M+Na]⁺ calcd for C₂₁H₃₁NO₆Na, 416.2044; found, 416.2060.

### Example 8D: Preparation of tert-butyl ((3S,8S,9S,10S)-10-methyl-8,9-bis((2-methylallyl)oxy)-2-oxo-1,6-dioxecan-3-yl)carbamate (Cmpd 257)

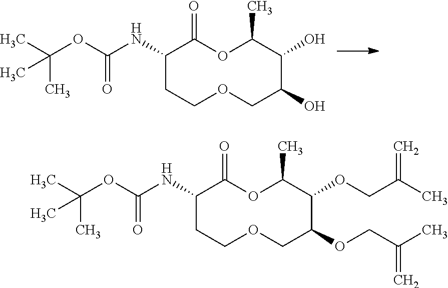 [SMILES: C[C@@H]1OC(=O)[C@@H](NC(=O)OC(C)(C)C)CCOC[C@H](O)[C@H]1O ; C=C(C)CO[C@H]1[C@H](C)OC(=O)[C@@H](NC(=O)OC(C)(C)C)CCOC[C@@H]1OCC(=C)C]

To a solution of tert-butyl ((3S,8S,9R,10S)-8,9-dihydroxy-10-methyl-2-oxo-1,6-dioxecan-3-yl)carbamate (290 mg, 0.908 mmol) in THF (9 mL) were added dppf (50 mg, 0.091 mmol) and Pd₂dba₃ (42 mg, 0.045 mmol), and the resulting solution was heated to 60° C., treated with methallyl tert-butyl carbonate (148 mg, 0.86 mmol), and stirred for 20 min at 60° C. The reaction mixture was treated with a second portion of methallyl tert-butyl carbonate (148 mg, 0.86 mmol), stirred for an additional 20 min at 60° C., treated with a third portion of methallyl tert-butylcarbonate (234 mg, 1.36 mmol), and stirred for an additional 40 min at 60° C. The reaction mixture was concentrated and the residue purified by column chromatography (SiO₂, 2→20% acetone in hexanes) to provide the title compound (229 mg, 47%) as a sticky, yellow wax: ¹H NMR (400 MHz, CDCl₃) δ 5.49 (d, J=6.3 Hz, 1H), 5.03-4.93 (m, 3H), 4.88-4.83 (m, 2H), 4.37-4.25 (m, 2H), 4.05-3.87 (m, 3H), 3.87-3.77 (m, 1H), 3.52 (dd, J=9.7, 7.8 Hz, 1H), 3.43 (dd, J=9.7, 1.6 Hz, 1H), 3.38-3.32 (m, 1H), 3.31-3.19 (m, 2H), 2.47 (t, J=13.4 Hz, 1H), 1.78 (d, J=16.7 Hz, 1H), 1.74 (t, J=1.1 Hz, 3H), 1.72 (t, J=1.1 Hz, 3H), 1.44 (s, 9H), 1.36 (d, J=6.3 Hz, 3H); ESIMS m/z 450.4 ([M+Na]⁺).

### Example 8E, Step 1: Preparation of tert-butyl N-tert-butoxycarbonyl-N-[(3S,8S,9S,10S)-10-methyl-8,9-bis(2-methylallyloxy)-2-oxo-1,5-dioxecan-3-yl]carbamate (Cmpd 194)

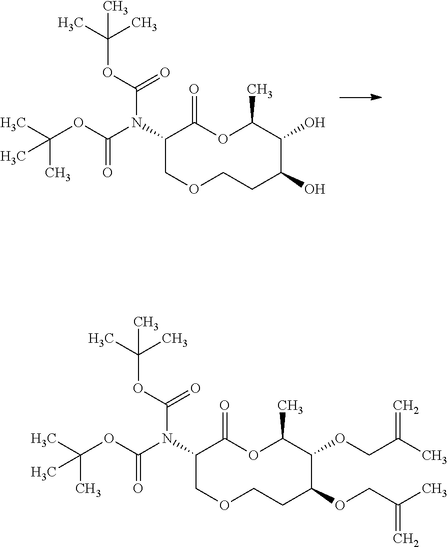 [SMILES: C[C@@H]1OC(=O)[C@@H](N(C(=O)OC(C)(C)C)C(=O)OC(C)(C)C)COCC[C@H](O)[C@H]1O ; C=C(C)CO[C@H]1[C@H](C)OC(=O)[C@@H](N(C(=O)OC(C)(C)C)C(=O)OC(C)(C)C)COCC[C@@H]1OCC(=C)C]

A solution of tert-butyl N-tert-butoxycarbonyl-N-[(3S,8S,9R,10S)-8,9-dihydroxy-10-methyl-2-oxo-1,5-dioxecan-3-yl]carbamate (124 mg, 0.296 mmol) in anhydrous THF (3 mL) was deoxygenated by brief application of vacuum and backfilling with N₂ (3×). The solution was treated with Pd₂dba₃ (13 mg, 0.015 mmol), dppf (16 mg, 0.030 mmol), and bis(2-methylallyl) carbonate (151 mg, 0.887 mmol), and the deoxygenation process was repeated. The reaction mixture was heated to and stirred at 60° C. for 18 h. The mixture was cooled to room temperature, diluted with H₂O (20 mL), extracted with EtOAc (3×20 mL), and the combined organic extracts were dried over MgSO₄, filtered, and concentrated to provide a black oil, which was shown to be a mixture of mono-allylated products (90 mg). This mixture of mono-allylated products was combined with a different lot of SM (131 mg, 0.312 mmol) and dissolved in anhydrous THF (5 mL). To this solution were added dimethylallyl carbonate (535 mg, 2.40 mmol), dppf (55 mg, 0.1 mmol), and Pd₂dba₃ (46 mg, 0.20 mmol) The resulting solution was heated to and stirred at 50° C. for 1.5 h, treated with a second portion of dimethylallyl carbonate (200 mg, 0.934 mmol), dppf (27 mg, 0.049 mmol) and Pd₂dba₃ (23 mg, 0.10 mmol), and then stirred at 50° C. for an additional 1 h. The reaction mixture was cooled to room temperature, concentrated, and purified by column chromatography (SiO₂, acetone in hexanes gradient) to provide the desired product as a mixture with dba. The mixture was purified a second time by column chromatography (SiO₂, 1.5→5% EtOAc in CH₂Cl₂) to give the title compound (116 mg, 36%) as a colorless oil: IR (Thin Film) 3076, 2979, 2935, 2872, 1759, 1708, 1456, 1367, 1317, 1248, 1145, 1123 cm⁻¹; ¹H NMR (400 MHz, CDCl₃) δ 5.09 (dd, J=6.0, 2.4 Hz, 1H), 5.03-4.91 (m, 3H), 4.85 (d, J=12.7 Hz, 2H), 4.35 (d, J=12.0 Hz, 1H), 4.21 (d, J=12.5 Hz, 1H), 4.05-3.97 (m, 1H), 3.88 (dt, J=12.0, 3.2 Hz, 3H), 1.56-1.47 (m, 18H), 3.79 (ddd, J=11.9, 9.4, 2.9 Hz, 1H), 3.59-3.45 (m, 2H), 3.22 (t, J=8.2 Hz, 1H), 2.05-1.93 (m, 1H), 1.74 (s, 3H), 1.71 (s, 4H), 1.38 (d, J=6.3 Hz, 3H); ¹³C NMR (101 MHz, CDCl₃) δ 167.51, 152.65, 142.77, 142.34, 112.15, 111.72, 83.94, 82.81, 78.71, 76.87, 76.22, 73.66, 70.05, 68.74, 59.19, 35.26, 27.98, 19.83, 19.71, 18.74.

### Example 8E, Step 2: Preparation of tert-butyl N-tert-butoxycarbonyl-N-[(3S,8S,9S,10S)-8,9-diisobutoxy-10-methyl-2-oxo-1,5-dioxecan-3-yl]carbamate

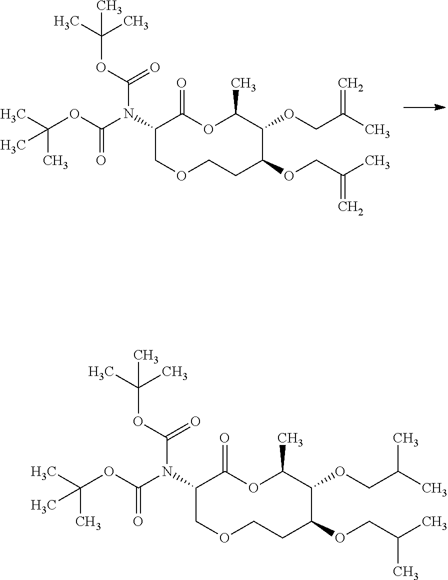

A high pressure steel reactor was charged with a solution of tert-butyl N-tert-butoxycarbonyl-N-[(3S,8S,9S,10S)-10-methyl-8,9-bis(2-methylallyloxy)-2-oxo-1,5-dioxecan-3-yl]carbamate (115 mg, 0.218 mmol) in EtOAc (10 mL) and Pd/C (10%, 23 mg, 0.022 mmol), and the reactor was pressurized with 600 psi H₂. The reaction mixture was warmed to and stirred at 40° C. for 16 h, cooled to room temperature, and filtered through a plug of Celite®. The filtrate was concentrated to provide the title compound contaminated with 20% of the mono-Boc impurity, tert-butyl ((3S,8S,9S,10S)-8,9-diisobutoxy-10-methyl-2-oxo-1,5-dioxecan-3-yl)carbamate, an inconsequential by-product that will converge upon deprotection (110 mg, 95%): IR (Thin Film) 2957, 2933, 2872, 1753, 1710, 1470, 1367, 1319, 1249, 1146, 1123, 1089 cm⁻¹; HRMS-ESI (m/z) [M+Na]⁺ calcd for C₂₇H₄₉NNaO₉, 554.3300; found, 554.3303.

### Example 8F: Preparation of (3S,6S,7S,8S)-8-butoxy-3-((tert-butoxycarbonyl)amino)-6-methyl-4-oxo-1,5-dioxecan-7-yl benzoate (Cmpd 193)

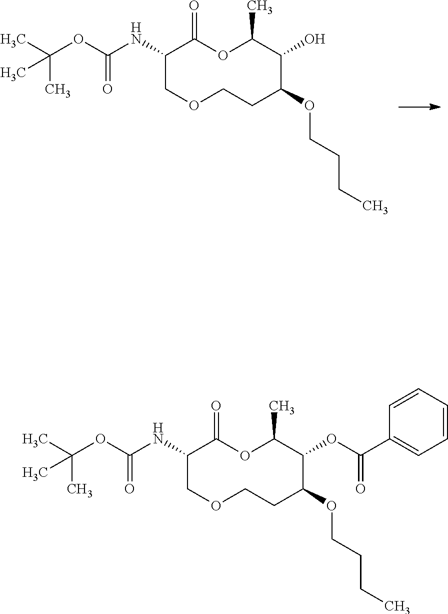

To a solution of tert-butyl ((3S,8S,9S,10S)-8-butoxy-9-hydroxy-10-methyl-2-oxo-1,5-dioxecan-3-yl)carbamate (131 mg, 0.349 mmol) in anhydrous pyridine (2.1 mL) at 0° C. were added DMAP (8.0 mg, 0.070 mmol) and benzoyl chloride (81 μL, 0.67 mmol), and the resulting solution was removed from the cold bath and stirred at room temperature overnight. The reaction mixture was quenched with H₂O (2 mL), stirred at room temperature for 15 min, partitioned between Et₂O (20 mL) and H₂O (20 mL), and the phases separated. The aq phase was extracted with Et₂O (2×20 mL), and the organic extracts were combined, washed with brine (20 mL), dried over MgSO₄, filtered, and concentrated to provide a yellow oil, which was purified by column chromatography (SiO₂; 5→35% EtOAc in hexanes) to give the title compound (151 mg, 90%) as a sticky solid: ¹H NMR (400 MHz, CDCl₃) δ 8.19-7.99 (m, 2H), 7.64-7.52 (m, 1H), 7.46 (td, J=7.6, 4.5 Hz, 2H), 5.73 (d, J=8.1 Hz, 1H), 5.62-5.42 (m, 1H), 5.05 (t, J=8.7 Hz, 1H), 4.61 (dd, J=8.1, 2.3 Hz, 1H), 3.98-3.70 (m, 3H), 3.61-3.32 (m, 4H), 2.32-2.10 (m, 1H), 1.79-1.57 (m, 1H), 1.54-1.43 (m, 9H), 1.38 (d, J=6.4 Hz, 3H), 1.34-0.97 (m, 4H), 0.66 (t, J=7.3 Hz, 3H); ¹³C NMR (101 MHz, CDCl₃) δ 169.92, 165.54, 155.58, 133.48, 133.18, 130.11, 129.82, 129.71, 128.43, 128.41, 80.04, 76.65, 75.87, 73.08, 71.97, 67.85, 67.27, 55.38, 33.78, 32.00, 28.33, 18.99, 18.75, 13.71; HRMS-ESI (m/z) [M+Na]⁺ calcd for C₂₅H₃₇NO₈Na, 502.2411; found, 502.2421.

### Example 8G, Step 1: Preparation of provide tert-butyl ((3S,8S,9R,10S)-10-methyl-9-((2-methylallyl)oxy)-2-oxo-8-propyloxecan-3-yl)carbamate (Cmpd 239)

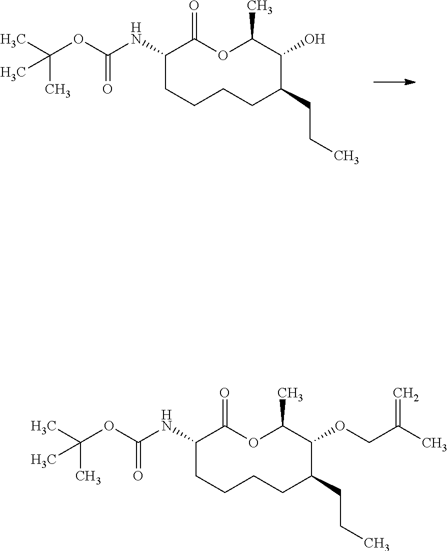

A solution of tert-butyl ((3S,8S,9R,10S)-9-hydroxy-10-methyl-2-oxo-8-propyloxecan-3-yl)carbamate (150 mg, 0.437 mmol), dppf (24 mg, 0.044 mmol), and tert-butyl(2-methylallyl) carbonate (150 mg, 0.873 mmol) in THF (4.4 mL) was degassed by evacuating the flask under vacuum and backfilling with N₂ (3×). The mixture was treated with Pd₂dba₃ (20 mg, 0.022 mmol) and the reaction mixture was warmed to and stirred at 60° C. for 1 h. The resulting orange solution was cooled to room temperature, concentrated, and the residue purified by column chromatography (SiO₂; 2→20% acetone in hexanes) to provide the title compound (140 mg, 81%) as a clear, colorless oil: ¹H NMR (400 MHz, CDCl₃) δ 5.35-5.21 (m, 1H), 5.01-4.96 (m, 1H), 4.96-4.89 (m, 1H), 4.88-4.83 (m, 1H), 4.34 (s, 1H), 3.94 (s, 2H), 3.00 (t, J=9.2 Hz, 1H), 2.05 (d, J=5.2 Hz, 1H), 1.97-1.83 (m, 1H), 1.75 (s, 3H), 1.62 (dtd, J=14.4, 6.0, 2.8 Hz, 1H), 1.54-1.46 (m, 3H), 1.45 (s, 9H), 1.43-1.38 (m, 3H), 1.36 (d, J=6.3 Hz, 3H), 1.31-1.22 (m, 1H), 1.21-1.03 (m, 3H), 0.88 (t, J=7.2 Hz, 3H); ¹³C NMR (101 MHz, CDCl₃) δ 172.88, 155.20, 141.97, 111.90, 85.05, 79.69, 76.40, 75.38, 52.77, 42.95, 34.51, 28.35, 27.35, 27.12, 24.94, 21.71, 20.14, 19.85, 18.53, 14.42; ESIMS m/z 420.4 ([M+Na]⁺).

### Example 8G, Step 2: Preparation of tert-butyl ((3S,8S,9R,10S)-9-isobutoxy-10-methyl-2-oxo-8-propyloxecan-3-yl)carbamate (Cmpd 245)

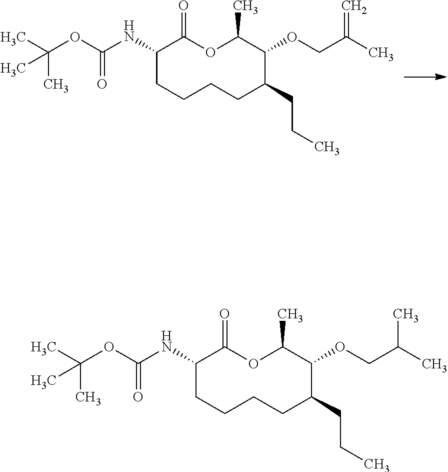

To a solution of tert-butyl ((3S,8S,9R,10S)-10-methyl-9-((2-methylallyl)oxy)-2-oxo-8-propyloxecan-3-yl)carbamate (128 mg, 0.322 mmol) in EtOAc (3.2 mL) was added Pd/C (5%, 34 mg, 0.016 mmol) and the reaction vessel was evacuated under vacuum and backfilled with H₂ (3×). The reaction mixture was placed under approximately 1 Atm of H₂ (balloon), stirred overnight at room temperature, filtered, and concentrated to provide the title compound (130 mg, 96%) as a clear, colorless oil: ¹H NMR (400 MHz, CDCl₃) δ 5.41-5.22 (m, 1H), 4.89 (dq, J=9.2, 6.3 Hz, 1H), 4.34 (s, 1H), 3.39-3.22 (m, 2H), 2.91 (t, J=9.3 Hz, 1H), 2.17-1.99 (m, 1H), 1.85 (ddq, J=19.8, 13.2, 6.6 Hz, 2H), 1.62 (tdd, J=12.9, 5.7, 3.7 Hz, 1H), 1.54-1.38 (m, 14H), 1.35 (d, J=6.3 Hz, 3H), 1.27 (tdd, J=16.2, 6.4, 2.9 Hz, 2H), 1.12 (dtt, J=19.0, 9.7, 4.0 Hz, 3H), 0.92 (d, J=6.7 Hz, 6H), 0.89 (t, J=7.3 Hz, 3H); ¹³C NMR (101 MHz, CDCl₃) δ 172.82, 155.16, 84.64, 79.85, 79.56, 79.35, 75.55, 52.76, 42.93, 34.37, 29.09, 28.31, 27.43, 27.05, 24.97, 21.63, 20.15, 19.50, 19.46, 18.51, 14.39; ESIMS m/z 422.34 ([M+Na]⁺).

### Example 8H: Preparation of tert-butyl ((3S,8R,9R,10S)-8-(cyclopentylmethyl)-9-methoxy-10-methyl-2-oxooxecan-3-yl)carbamate (Cmpd 274)

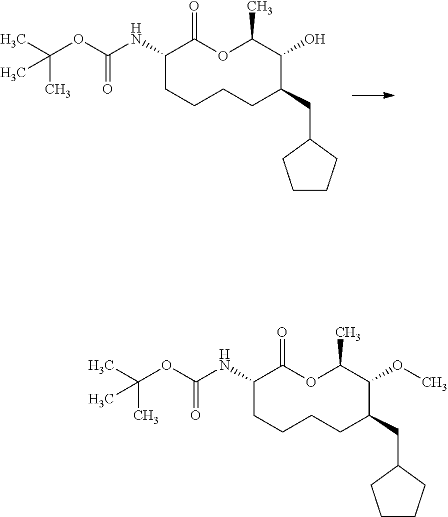 [SMILES: C[C@@H]1OC(=O)[C@@H](NC(=O)OC(C)(C)C)CCCC[C@H](CC2CCCC2)[C@H]1O ; CO[C@@H]1[C@@H](CC2CCCC2)CCCC[C@H](NC(=O)OC(C)(C)C)C(=O)O[C@H]1C]

To an oven-dried Schlenk flask was added a solution of tert-butyl ((3S,8R,9R,10S)-8-(cyclopentylmethyl)-9-hydroxy-10-methyl-2-oxooxecan-3-yl)carbamate (240 mg, 0.626 mmol) in anhydrous CH₂Cl₂ (6 mL), and the solution was cooled to 0° C. under N₂ and treated with N1,N1,N8,N8-tetramethylnaphthalene-1,8-diamine (201 mg, 0.939 mmol) and trimethyl-oxonium tetrafluoroborate (131 mg, 0.688 mmol). The resulting mixture was stirred for 30 min at 0° C., removed from the cold bath, allowed to warm to room temperature, and stirred at room temperature overnight. The mixture was diluted with CH₂Cl₂ (25 mL), washed with 1N HCl (2×10 mL), dried over Na₂SO₄, filtered, and concentrated. The residue was purified by column chromatography (SiO₂; 2→20% acetone in hexanes) to give the title compound (187 mg, 75%) as a sticky, colorless oil: ¹H NMR (400 MHz, CDCl₃) δ 5.29 (d, J=7.7 Hz, 1H), 4.88 (dq, J=9.1, 6.3 Hz, 1H), 4.33 (s, 1H), 3.43 (s, 3H), 2.82 (t, J=9.1 Hz, 1H), 2.05 (d, J=4.3 Hz, 1H), 1.88 (dt, J=16.1, 8.1 Hz, 2H), 1.82-1.64 (m, 3H), 1.59 (ddd, J=12.1, 7.0, 3.2 Hz, 3H), 1.55-1.46 (m, 5H), 1.45 (s, 9H), 1.37 (d, J=6.3 Hz, 3H), 1.19 (ddt, J=20.9, 11.2, 6.3 Hz, 3H), 1.12-0.97 (m, 3H); ¹³C NMR (101 MHz, CDCl₃) δ 172.95, 155.18, 87.19, 79.69, 75.20, 60.32, 52.78, 42.04, 38.48, 37.33, 33.86, 32.05, 28.34, 27.33, 27.13, 25.16, 25.07, 24.68, 21.88, 18.43; ESIMS m/z 420.4 ([M+Na]⁺).

### Example 81: Preparation of tert-butyl N-tert-butoxycarbonyl-N-[((3S,8S,9S,10S)-9-hydroxy-8-methoxy-10-methyl-2-oxooxecan-3-yl)]carbamate and tert-butyl N-tert-butoxycarbonyl-N-[((3S,8S,9S,10S)-8,9-dimethoxy-10-methyl-2-oxooxecan-3-yl)]carbamate (Cmpd 199 and Cmpd 200)

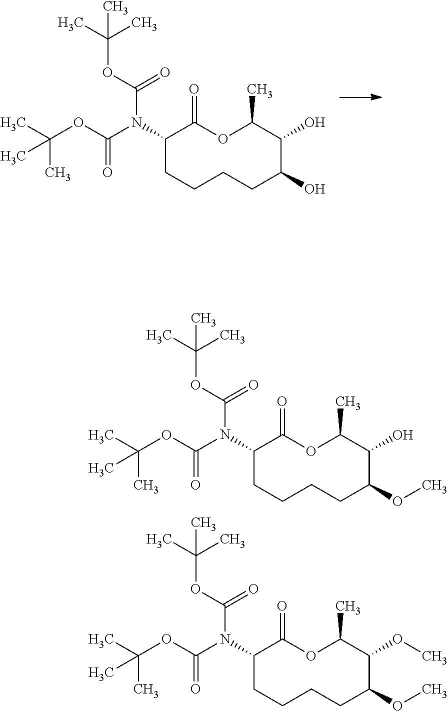

To a solution of tert-butyl N-tert-butoxycarbonyl-N-[(3S,8S,9R,10S)-8,9-dihydroxy-10-methyl-2-oxo-oxecan-3-yl]carbamate (0.468 g, 1.12 mmol) in anhydrous CH₂Cl₂ (11 mL) at 0° C. (icewater bath) were added N1,N1,N8,N8-tetramethylnaphthalene-1,8-diamine (0.721 g, 3.36 mmol) and trimethyloxonium tetrafluoroborate (0.332 g, 2.24 mmol), and the resulting suspension was stirred at 0° C. for 30 min, removed from the cold bath and warmed to room temperature, and stirred at room temperature for 2 h. The reaction mixture was poured into a mixture of H₂O (20 mL) and sat'd NaHCO₃ solution (20 mL) and extracted with CH₂Cl₂ (2×40 mL). The organic extracts were combined, washed with 1N HCl (40 mL), dried over Na₂SO₄, filtered, and concentrated to provide an oil which was purified by column chromatography (SiO₂) to provide the title compounds:

tert-butyl N-tert-butoxycarbonyl-N-[((3S,8S,9S,10S)-9-hydroxy-8-methoxy-10-methyl-2-oxooxecan-3-yl)]carbamate (280 mg, 58%) was isolated as a clear, colorless oil: IR (Thin Film) 3530.70, 2978.80, 2936.02, 1740.74, 1701.07, 1359.35, 1142.11 cm⁻¹; ¹H NMR (400 MHz, CDCl₃) δ 5.02 (t, J=4.7 Hz, 1H), 4.65 (dq, J=9.4, 6.1 Hz, 1H), 3.51-3.41 (m, 1H), 3.39 (s, 3H), 3.14 (d, J=0.9 Hz, 1H), 2.95 (ddd, J=8.4, 6.2, 2.0 Hz, 1H), 2.27-2.12 (m, 1H), 1.97-1.70 (m, 3H), 1.70-1.59 (m, 1H), 1.55-1.46 (m, 1H), 1.51 (s, 18H), 1.43 (d, J=6.1 Hz, 3H), 1.34-1.27 (m, 2H); HRMS-ESI (m/z) [M+Na]⁺ calcd for C₂₁H₃₇NO₈Na, 454.2411; found, 454.2418; and

tert-butyl N-tert-butoxycarbonyl-N-[((3S,8S,9S,10S)-8,9-dimethoxy-10-methyl-2-oxooxecan-3-yl)]carbamate (24 mg, 4.8%) was isolated as a clear, colorless oil: IR (Thin Film) 2978.32, 2933.32, 2826.06, 1741.76, 1702.58, 1366.30, 1104.85 cm⁻¹; ¹H NMR (400 MHz, CDCl₃) δ 4.82 (dd, J=6.9, 3.3 Hz, 1H), 4.71 (dq, J=7.7, 6.3 Hz, 1H), 3.51 (s, 3H), 3.41 (s, 3H), 3.26 (ddd, J=7.8, 5.9, 2.0 Hz, 1H), 3.15 (t, J=7.7 Hz, 1H), 2.25-2.12 (m, 1H), 2.03-1.92 (m, 1H), 1.82-1.60 (m, 4H), 1.51 (s, 18H), 1.40 (d, J=6.3 Hz, 3H), 1.34-1.23 (m, 2H); HRMS-ESI (m/z) [M+Na]⁺ calcd for C₂₂H₃₉NO₈Na, 468.2568; found, 468.2577.

### Example 8J: Preparation of tert-butyl N-tert-butoxycarbonyl-N-[((3aS,4S,7S,11aS)-2,2,4-trimethyl-6-oxooctahydro-3aH-[1,3]dioxolo[4,5-c]oxecin-7-yl)]carbamate (Cmpd 224)

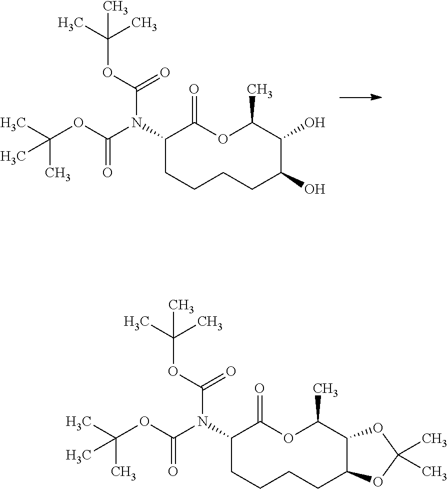 [SMILES: C[C@@H]1OC(=O)[C@@H](N(C(=O)OC(C)(C)C)C(=O)OC(C)(C)C)CCCC[C@H](O)[C@H]1O ; C[C@@H]1OC(=O)[C@@H](N(C(=O)OC(C)(C)C)C(=O)OC(C)(C)C)CCCC[C@@H]2OC(C)(C)O[C@@H]12]

To a solution of tert-butyl N-tert-butoxycarbonyl-N-[(3S,8S,9R,10S)-8,9-dihydroxy-10-methyl-2-oxo-oxecan-3-yl]carbamate (230 mg, 0.551 mmol) in 2,2-dimethoxypropane (2.0 mL, 16 mmol) was added p-toluenesulfonic acid (9.5 mg, 0.055 mmol), and the resulting mixture was stirred at room temperature for 2 d, The reaction mixture was concentrated and the resulting oil was purified by column chromatography (SiO₂, 0→20% acetone in hexanes) to provide the title compound (226 mg, 90%) as a clear oil: IR (Thin Film) 2980.26, 2935.02, 1739.97, 1703.53 cm⁻¹; ¹H NMR (300 MHz, CDCl₃) δ 5.02 (dd, J=7.5, 6.0 Hz, 1H), 4.74 (dq, J=9.4, 6.1 Hz, 1H), 3.95 (td, J=8.3, 3.7 Hz, 1H), 3.59 (dd, J=9.4, 7.8 Hz, 1H), 2.22-2.06 (m, 1H), 2.04-1.84 (m, 2H), 1.75-1.58 (m, 4H), 1.50 (s, 18H), 1.45-1.37 (m, 7H), 1.34 (s, 3H); HRMS-ESI (m/z) [M+Na]⁺ calcd for C₂₃H₃₉NO₈Na, 480.2568; found, 480.2541.

### Example 9, Step 1: Preparation of (S)-methyl 2-((S)-1-hydroxyethyl)-5-methylhex-4-enoate

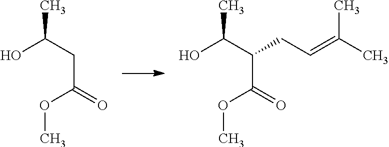

To a solution of diisopropylamine (19.9 mL, 142 mmol) in anhydrous THF (99 mL) at −50° C. was added n-BuLi (54.3 mL, 130 mmol, 2.5 M in hexanes), and the resulting solution was removed from the cold bath for 15 min and then cooled back to −50° C. To the freshly prepared LDA was added a solution of (S)-methyl 3-hydroxybutanoate (6.64 mL, 59.3 mmol) in THF (20.0 mL) dropwise over a 15 min period, and the mixture was allowed to warm to −30° C. over a 30 min period. The reaction mixture was stirred at −30° C. for 1 h, cooled to −78° C., and the resulting enolate was treated with a solution of 1-bromo-3-methylbut-2-ene (13.7 mL, 119 mmol) in anhydrous 1,2-dimethoxyethane (20.0 mL, 193 mmol) dropwise over a 15 min period. The mixture was stirred between −60° C. and −70° C. for 1 h, and the reaction flask was removed from the bath and stirring continued as the mixture was warmed to room temperature over a period of 1.5 h. The reaction was quenched by the addition of sat'd aq NH₄Cl (50 mL) and extracted with EtOAc (50 mL). The phases were separated and the aq phase was further extracted with EtOAc (2×50 mL), and the combined organic extracts were washed with brine (50 mL), dried over Na₂SO₄, filtered, and concentrated to dryness. The crude residue was purified by column chromatography (SiO₂; 0→40% EtOAc in hexanes) to afford the title compound (9.5 g, 86%) as a slightly yellow oil: IR (Thin Film) 3452, 2971, 2929, 1730, 1437, 1198, 1160 cm⁻¹; ¹H NMR (400 MHz, CDCl₃) δ 5.11-5.01 (m, 1H), 3.92 (p, J=6.3 Hz, 1H), 3.70 (s, 3H), 2.78 (s, 1H), 2.46-2.28 (m, 3H), 1.69 (d, J=1.4 Hz, 3H), 1.62 (s, 3H), 1.23 (d, J=6.4 Hz, 3H); ¹³C NMR (101 MHz, CDCl₃) δ 175.54, 134.14, 120.30, 67.78, 52.72, 51.52, 27.90, 25.73, 21.46, 17.64.

### Example 9, Step 2: Preparation of (S)-methyl 2-((S)-1-hydroxyethyl)-5-methylhexanoate

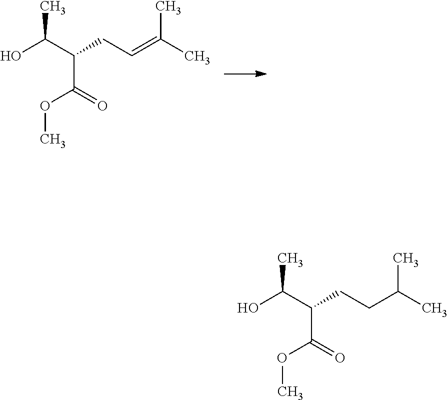

To a well stirred solution of (S)-methyl 2-((S)-1-hydroxyethyl)-5-methylhex-4-enoate (9.5 g, 51.0 mmol) in MeOH (51 mL) was added Pd/C (10%, 0.543 g, 5.10 mmol), and the mixture was placed under approximately 1 Atm (balloon) of H₂, and stirred at room temperature for 20 h. The reaction mixture was filtered through a plug of Celite® and the plug was washed with MeOH (20 mL). The filtrate and washings were combined and concentrated, and the concentrate was diluted with CH₂Cl₂ (50 mL), dried by passing through phase separator cartridge, and concentrated to give the title compound (9.45 g, 98%) as a slightly yellow oil: IR (Thin Film) 3451, 2954, 2871, 1736, 1719, 1169 cm⁻¹; ¹H NMR (400 MHz, CDCl₃) δ 3.91 (p, J=6.4 Hz, 1H), 3.72 (s, 3H), 2.77 (s, 1H), 2.36 (ddd, J=9.2, 6.3, 5.0 Hz, 1H), 1.72-1.45 (m, 3H), 1.28-1.05 (m, 5H), 0.88 (dd, J=6.6, 3.2 Hz, 6H); ¹³C NMR (75 MHz, CDCl₃) δ 176.13, 68.55, 53.29, 51.67, 36.55, 28.16, 27.37, 22.74, 22.44, 21.68.

### Example 9, Step 3: Preparation of (S)-methyl 2-((S)-1-((4-methoxybenzyl)oxy)ethyl)-5-methylhexanoate

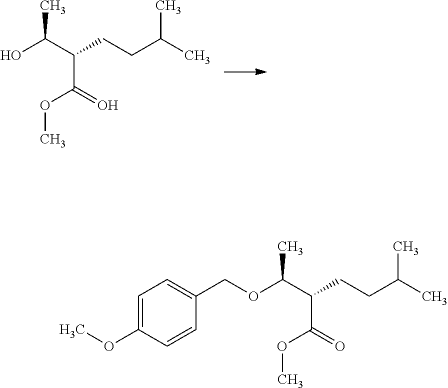 [SMILES: COC(=O)[C@@H](CCC(C)C)[C@H](C)O ; COC(=O)[C@@H](CCC(C)C)[C@H](C)OCc1ccc(OC)cc1]

To a solution of (S)-methyl 2-((S)-1-hydroxyethyl)-5-methylhexanoate (5.00 g, 26.6 mmol) and ((1S,4R)-7,7-dimethyl-2-oxobicyclo[2.2.1]heptan-1-yl)methanesulfonic acid (0.617 g, 2.66 mmol) in CH₂Cl₂ (53 mL) was added 4-methoxybenzyl 2,2,2-trichloroacetimidate (8.27 mL, 39.8 mmol) at 0° C., and the reaction mixture was removed from the cold bath, warmed to room temperature, and stirred for 17 h. The reaction mixture was diluted with hexanes (50 mL) and the resulting precipitate was removed by filtration and washed with hexanes (2×10 mL). To the combined filtrate and washings was added Celite® and the solvent was removed under reduced pressure. The resulting adsorbed material was purified by column chromatography (SiO₂; 0→35% EtOAc in hexanes) to afford the title compound (6.3 g, 77%) as a colorless oil: ¹H NMR (400 MHz, CDCl₃) δ 7.24-7.16 (m, 2H), 6.89-6.79 (m, 2H), 4.49 (d, J=11.2 Hz, 1H), 4.33 (d, J=11.1 Hz, 1H), 3.75 (s, 3H), 3.74-3.62 (m, 4H), 2.49 (ddd, J=10.7, 8.2, 4.0 Hz, 1H), 1.62-1.40 (m, 3H), 1.23-1.16 (m, 3H), 1.16-1.03 (m, 2H), 0.87 (d, J=3.9 Hz, 3H), 0.85 (d, J=3.9 Hz, 3H); ¹³C NMR (101 MHz, CDCl₃) δ 175.03, 159.10, 130.63, 129.14, 113.62, 76.16, 70.71, 55.11, 52.64, 51.25, 36.58, 27.97, 26.00, 22.69, 22.17, 17.08; ESIMS m/z 331 ([M+Na]⁺).

### Example 9, Step 4: Preparation of (3S,4R)-4-((S)-1-((4-methoxybenzyl)oxy)ethyl)-7-methyloct-1-en-3-ol and (3R,4R)-4-((S)-1-((4-methoxybenzyl)oxy)ethyl)-7-methyloct-1-en-3-ol

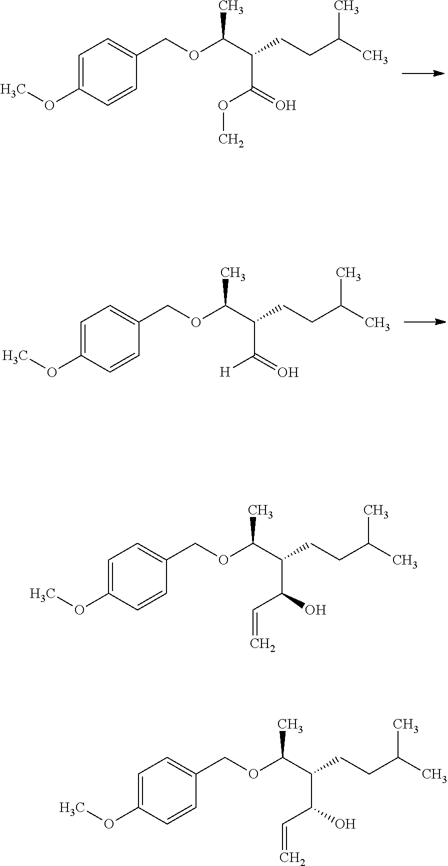

To a solution of (S)-methyl 2-((S)-1-((4-methoxybenzyl)oxy)ethyl)-5-methylhexanoate (6.00 g, 19.5 mmol) and chlorobis(cyclooctene)iridium(I) dimer (0.349 g, 0.389 mmol) in dry CH₂Cl₂ (20 mL) was slowly added Et₂SiH₂ (3.76 mL, 29.2 mmol) at 0° C., and the reaction mixture was removed from the cold bath and stirred at room temperature for 20 h under N₂. The reaction mixture was transferred via cannula to an ice-cooled mixture of Et₂O (60 mL) and 2 N HCl (20 mL) over a period of 15 min, and then warmed to and stirred at room temperature for 30 min. The phases were separated and the aq phase was further extracted with Et₂O (2×50 mL). The combined organics were washed with sat'd aq NaHCO₃ (25 mL) and brine (25 mL), dried over Na₂SO₄, filtered, treated with Celite®, and concentrated. The resulting adsorbed material was purified by column chromatography (SiO₂; 0→75% EtOAc in hexanes) to afford the intermediate aldehyde, (S)-2-((S)-1-((4-methoxybenzyl)oxy)ethyl)-5-methylhexanal, which was used immediately in the next step. The aldehyde was dissolved in THF (30 mL), and the solution was cooled to −78° C., treated slowly with vinylmagnesium bromide (29.2 mL, 29.2 mmol, 1 M in THF), stirred at −78° C. for 30 min, and warmed to and stirred at room temperature for 30 min. The reaction mixture was quenched by the addition of sat'd aq NH₄Cl (30 mL), the phases were separated, and the aq phase was further extracted with Et₂O (3×50 mL). The combined organics were dried over Na₂SO₄, filtered, and concentrated to dryness. The residue was dissolved in CH₂Cl₂ (20 mL), adsorbed to Celite®, and the adsorbed material was purified by column chromatography (SiO₂; 0→15% acetone in hexanes) to afford the title compounds:

(3S,4R)-4-((S)-1-((4-methoxybenzyl)oxy)ethyl)-7-methyloct-1-en-3-ol (2.35 g, 39%) was isolated as a colorless oil: ¹H NMR (400 MHz, CDCl₃) δ 7.27-7.19 (m, 2H), 6.91-6.82 (m, 2H), 5.84 (ddd, J=17.2, 10.6, 4.7 Hz, 1H), 5.29 (app dt, J=17.2, 1.9 Hz, 1H), 5.16 (app dt, J=10.6, 1.9 Hz, 1H), 4.58 (d, J=11.0 Hz, 1H), 4.53-4.45 (m, 1H), 4.27 (d, J=10.9 Hz, 1H), 3.83 (d, J=4.3 Hz, 1H), 3.79 (s, 3H), 3.76-3.65 (m, 1H), 1.52-1.26 (m, 7H), 1.20-1.06 (m, 2H), 0.85 (app dd, J=6.6, 2.2 Hz, 6H); ¹³C NMR (101 MHz, CDCl₃) δ 159.33, 139.16, 130.02, 129.54, 114.61, 113.88, 76.55, 72.08, 70.65, 55.26, 49.31, 37.35, 28.25, 23.51, 22.63, 22.52, 17.71; ESIMS m/z 329 ([M+Na+]⁺); and

(3R,4R)-4-((S)-1-((4-methoxybenzyl)oxy)ethyl)-7-methyloct-1-en-3-ol (1.48 g, 25%) was isolated as a colorless oil: ¹H NMR (400 MHz, CDCl₃) δ 7.30-7.22 (m, 2H), 6.91-6.83 (m, 2H), 5.89 (ddd, J=17.1, 10.3, 6.7 Hz, 1H), 5.24 (ddd, J=17.2, 1.8, 1.2 Hz, 1H), 5.12 (ddd, J=10.4, 1.8, 1.1 Hz, 1H), 4.58 (d, J=11.1 Hz, 1H), 4.35 (d, J=11.1 Hz, 1H), 4.22-4.13 (m, 1H), 3.80 (s, 3H), 3.70 (p, J=6.3 Hz, 1H), 3.66 (d, J=3.2 Hz, 1H), 1.56 (tt, J=6.8, 5.2 Hz, 1H), 1.49-1.24 (m, 6H), 1.19-1.08 (m, 2H), 0.84 (dd, J=6.7, 2.0 Hz, 6H); ¹³C NMR (101 MHz, CDCl₃) δ 159.24, 140.20, 130.22, 129.41, 115.17, 113.87, 78.10, 75.83, 70.43, 55.28, 48.98, 36.07, 28.53, 26.08, 22.52, 17.93; ESIMS m/z 329 ([M+Na]⁺).

### Example 9, Step 5: Preparation of tert-butyl ((3R,4S)-4-((S)-1-((4-methoxybenzyl)oxy)ethyl)-7-methyloct-1-en-3-yl) carbonate

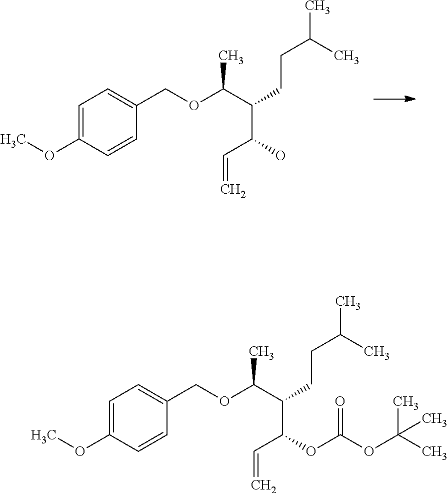

To a well-stirred solution of (3R,4R)-4-((S)-1-((4-methoxybenzyl)oxy)ethyl)-7-methyloct-1-en-3-ol (475 mg, 1.55 mmol) in THF (5.2 mL) at −78° C. was added n-BuLi (678 μL, 1.63 mmol, 2.4 M in hexanes), and the mixture was stirred at this temperature for 10 min, removed from the cold bath, and treated with a single portion of solid Boc₂O (372 mg, 1.71 mmol). The reaction mixture was warmed to and stirred at room temperature for 4 h and quenched by the addition of sat'd aq NH₄Cl (10 mL). The phases were separated and the aq phase was extracted with Et₂O (3×15 mL). The combined organics were dried over Na₂SO₄, filtered, and the filtrate was treated with Celite®, and concentrated. The adsorbed material was purified by column chromatography (SiO₂; 0→35% EtOAc in hexanes) to afford the title compound (370 mg, 59%) as a colorless oil: ¹H NMR (400 MHz, CDCl₃) δ 7.29-7.21 (m, 2H), 6.91-6.81 (m, 2H), 5.83 (ddd, J=17.0, 10.6, 6.2 Hz, 1H), 5.30-5.13 (m, 3H), 4.47 (d, J=11.4 Hz, 1H), 4.35 (d, J=11.4 Hz, 1H), 3.78 (s, 3H), 3.61 (qd, J=6.3, 4.7 Hz, 1H), 1.93-1.82 (m, 1H), 1.55-1.15 (m, 17H), 0.86 (app dd, J=6.6, 2.3 Hz, 6H); ¹³C NMR (101 MHz, CDCl₃) δ 158.98, 152.98, 135.25, 131.05, 129.05, 116.71, 113.67, 81.62, 77.94, 74.19, 69.93, 55.21, 46.64, 37.65, 28.32, 27.80, 23.49, 22.59, 22.53, 16.72; ESIMS m/z 429 ([M+Na]⁺).

### Example 9, Step 6: Preparation of (S)-benzyl 2-((tert-butoxycarbonyl)amino)-3-(((R,E)-4-((S)-1-((4-methoxybenzyl)oxy)ethyl)-7-methyloct-2-en-1-yl)oxy)propanoate

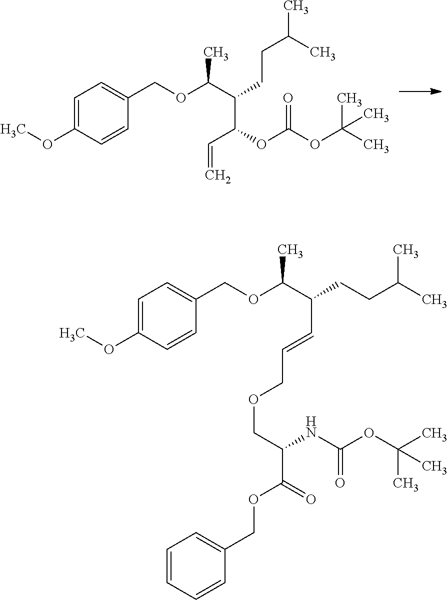

To a solution of (S)-benzyl 2-((tert-butoxycarbonyl)amino)-3-hydroxypropanoate (309 mg, 1.05 mmol), Pd₂(dba)₃ (57 mg, 0.063 mmol), and dppf (70 mg, 0.13 mmol) in THF (2 mL) was added a solution of tert-butyl ((3R,4S)-4-((S)-1-((4-methoxybenzyl)oxy)ethyl)-7-methyloct-1-en-3-yl)carbonate (340 mg, 0.836 mmol) in THF (3.6 mL), and the reaction mixture was heated to and stirred at 55° C. for 2.5 h. The mixture was cooled to room temperature, diluted with CH₂Cl₂ (5 mL), and the resulting solution was treated with Celite®. The solvent was removed under reduced pressure, and the adsorbed material was purified by column chromatography (SiO₂; 0→30% EtOAc in hexanes) to afford the title compound (300 mg, 62%): ¹H NMR (400 MHz, CDCl₃) δ 7.38-7.27 (m, 5H), 7.27-7.21 (m, 2H), 6.90-6.83 (m, 2H), 5.55-5.32 (m, 3H), 5.27 (d, J=12.5 Hz, 1H), 5.11 (d, J=12.4 Hz, 1H), 4.53-4.42 (m, 2H), 4.34 (d, J=11.5 Hz, 1H), 3.96-3.74 (m, 6H), 3.59 (dd, J=9.4, 3.3 Hz, 1H), 3.51-3.40 (m, 1H), 2.07-1.98 (m, 1H), 1.52-1.40 (m, 11H), 1.35-1.25 (m, 1H), 1.16-0.98 (m, 5H), 0.84 (app dd, J=6.9, 1.8 Hz, 6H); ¹³C NMR (101 MHz, CDCl₃) δ 170.71, 159.04, 155.56, 135.94, 135.59, 131.12, 129.16, 128.51, 128.25, 128.10, 127.55, 113.68, 79.90, 76.76, 72.11, 70.35, 69.44, 67.02, 55.26, 54.15, 48.59, 36.78, 28.33, 28.16, 28.11, 22.82, 22.49, 17.08; ESIMS m/z 606 ([M+Na]⁺).

### Example 9, Step 7: Preparation of (S)-benzyl 2-((tert-butoxycarbonyl)amino)-3-(((R,E)-4-((S)-1-hydroxyethyl)-7-methyloct-2-en-1-yl)oxy)propanoate

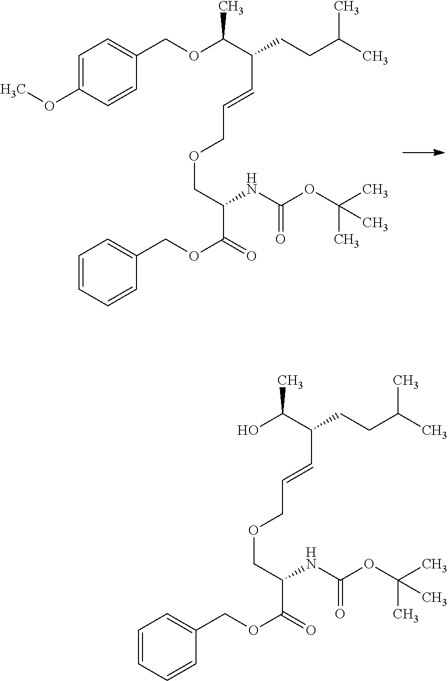 [SMILES: COc1ccc(CO[C@@H](C)[C@@H](/C=C/COC[C@H](NC(=O)OC(C)(C)C)C(=O)OCc2ccccc2)CCC(C)C)cc1 ; CC(C)CC[C@H](/C=C/COC[C@H](NC(=O)OC(C)(C)C)C(=O)OCc1ccccc1)[C@H](C)O]

To a solution of (S)-benzyl 2-((tert-butoxycarbonyl)amino)-3-(((R,E)-4-((S)-1-((4-methoxybenzyl)oxy)ethyl)-7-methyloct-2-en-1-yl)oxy)propanoate (295 mg, 0.505 mmol) in a mixture of H₂O (184 μL) and CH₂Cl₂ (1.84 mL) was added DDQ (120 mg, 0.531 mmol) at 0° C. The mixture was stirred vigorously at 0° C. for 1 h, treated with 1 N NaOH (531 μL, 0.531 mmol), and diluted with H₂O (6 mL). The phases were separated and the aq phase was extracted with CH₂Cl₂ (3×10 mL). The combined organics were washed with brine (8 mL), dried over Na₂SO₄, filtered, treated with Celite®, and concentrated under reduced pressure. The resulting adsorbed material was purified by column chromatography (SiO₂; 0→60% EtOAc/hexanes) to give the title compound (205 mg, 88%) as a colorless oil: ¹H NMR (400 MHz, CDCl₃) δ 7.39-7.29 (m, 5H), 5.49-5.37 (m, 3H), 5.28 (d, J=12.4 Hz, 1H), 5.12 (d, J=12.3 Hz, 1H), 4.52-4.43 (m, 1H), 4.04-3.75 (m, 3H), 3.70-3.58 (m, 2H), 1.93-1.81 (m, 1H), 1.69 (br s, 1H), 1.55-1.37 (m, 11H), 1.32-1.18 (m, 1H), 1.20-1.03 (m, 5H), 0.86 (app dd, J=6.6, 3.8 Hz, 6H); ¹³C NMR (101 MHz, CDCl₃) δ 170.67, 155.51, 135.52, 134.90, 129.08, 128.52, 128.29, 128.12, 79.97, 71.70, 69.88, 69.58, 67.08, 54.08, 50.80, 36.65, 28.59, 28.31, 28.03, 22.83, 22.35, 20.90; ESIMS m/z 486 ([M+Na]⁺).

### Example 9, Step 8: Preparation of (S)-2-((tert-butoxycarbonyl)amino)-3-(((R)-4-((S)-1-hydroxyethyl)-7-methyloctyl)oxy)propanoic acid

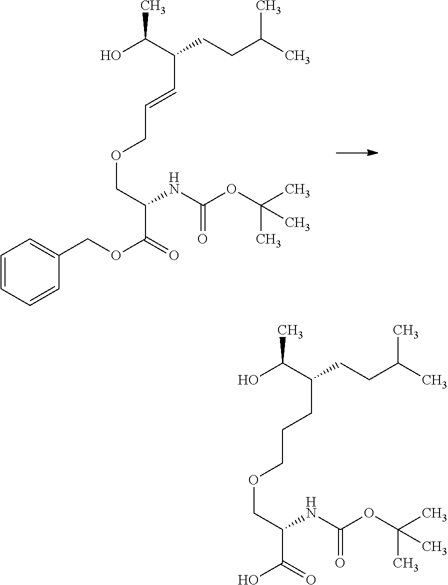 [SMILES: CC(C)CC[C@H](/C=C/COC[C@H](NC(=O)OC(C)(C)C)C(=O)OCc1ccccc1)[C@H](C)O ; CC(C)CC[C@H](CCCOC[C@H](NC(=O)OC(C)(C)C)C(=O)O)[C@H](C)O]

To a solution of (S)-benzyl 2-((tert-butoxycarbonyl)amino)-3-(((R,E)-4-((S)-1-hydroxyethyl)-7-methyloct-2-en-1-yl)oxy)propanoate (205 mg, 0.442 mmol) in EtOAc (2.2 mL) was added Pd/C (10%, 47 mg, 0.04 mmol), and the resulting mixture was placed under approximately 1 Atm of H₂ (balloon pressure) and stirred at room temperature for 18 h. The mixture was placed under a stream of N₂ to remove the H₂ and was then filtered through a plug of Celite®. The plug was washed with CH₂Cl₂ (2×5 mL) and the combined organics were concentrated to give the title compound (160 mg, 96%) as a colorless film: ¹H NMR (400 MHz, CDCl₃) δ 7.23-7.11 (m, 2H), 5.49 (d, J=8.5 Hz, 1H), 4.44 (dt, J=9.5, 3.4 Hz, 1H), 3.87 (dd, J=9.2, 3.4 Hz, 1H), 3.83-3.76 (m, 1H), 3.67 (dd, J=9.4, 3.4 Hz, 1H), 3.51 (dt, J=9.4, 5.8 Hz, 1H), 3.42 (ddt, J=9.4, 4.0, 2.3 Hz, 1H), 1.63-1.29 (m, 17H), 1.19-1.12 (m, 5H), 0.88 (app dd, J=6.6, 1.9 Hz, 6H); ¹³C NMR (101 MHz, CDCl₃) δ 174.03, 155.68, 80.13, 71.56, 70.56, 70.02, 53.90, 44.35, 36.18, 28.46, 28.32, 27.19, 26.65, 25.60, 22.72, 22.55, 20.01; ESIMS m/z 374 ([M−H]⁻).

### Example 9, Step 9: Preparation of tert-butyl ((3S,9R,10S)-9-isopentyl-10-methyl-2-oxo-1,5-dioxecan-3-yl)carbamate (Cmpd 253)

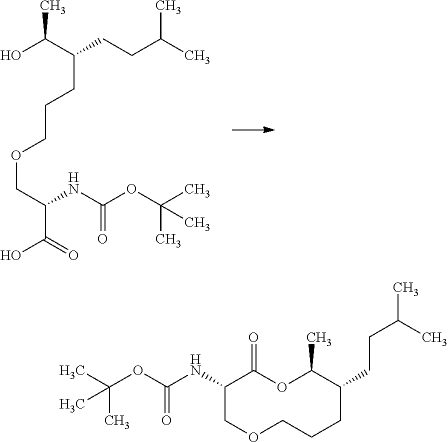 [SMILES: CC(C)CC[C@H](CCCOC[C@H](NC(=O)OC(C)(C)C)C(=O)O)[C@H](C)O ; CC(C)CC[C@@H]1CCCOC[C@H](NC(=O)OC(C)(C)C)C(=O)O[C@H]1C]

To a stirred solution of MNBA (279 mg, 0.810 mmol) and DMAP (297 mg, 2.43 mmol) in toluene (41 mL) was added via a syringe pump a solution of (S)-2-((tert-butoxycarbonyl)amino)-3-(((R)-4-((S)-1-hydroxyethyl)-7-methyloctyl)oxy)propanoic acid (152 mg, 0.405 mmol) in anhydrous toluene (22 mL, 0.02 M) over a 7 h period at 68° C., and the reaction mixture was stirred at 68° C. for an additional 1 h. The mixture was cooled to and stirred room temperature for 15 h and concentrated, and the residue was dissolved in CH₂Cl₂ (15 mL) and treated with Celite®. The solvent was evaporated under reduced pressure and the adsorbed material was purified by column chromatography (SiO₂; 0→40% EtOAc in hexanes) to give the title compound (49 mg, 34%) as a colorless oil: ¹H NMR (400 MHz, CDCl₃) δ 5.51 (d, J=7.9 Hz, 1H), 5.03 (dq, J=9.2, 6.2 Hz, 1H), 4.48 (ddd, J=8.0, 3.6, 1.7 Hz, 1H), 3.90-3.76 (m, 2H), 3.54 (ddd, J=11.4, 9.7, 3.4 Hz, 1H), 3.39 (ddd, J=11.4, 4.8, 3.6 Hz, 1H), 1.71-1.00 (m, 22H), 0.88 (app t, J=6.5 Hz, 6H); ¹³C NMR (101 MHz, CDCl₃) δ 170.37, 155.54, 79.74, 76.49, 69.20, 68.51, 55.36, 45.51, 35.63, 30.77, 28.33, 28.30, 27.81, 25.98, 22.87, 22.27, 19.88; ESIMS m/z 380 ([M+Na]⁺).

### Example 10, Step 1: Preparation of (2S,3R,4S)-4-benzyl-5-(2,2-diethoxyethoxy)-3-isobutoxypentan-2-ol

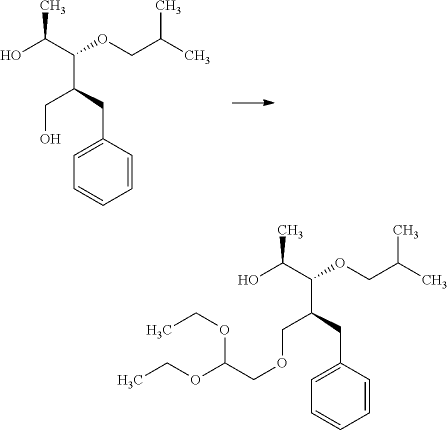 [SMILES: CC(C)CO[C@H]([C@H](CO)Cc1ccccc1)[C@H](C)O ; CCOC(COC[C@H](Cc1ccccc1)[C@@H](OCC(C)C)[C@H](C)O)OCC]

To a 1 dram vial equipped with a magnetic stir bar were added NaOH (0.18 g, 4.5 mmol), H₂O (1 mL), methyltributylammonium chloride (0.018 g, 0.075 mmol) and 2-bromo-1,1-diethoxy-ethane (0.092 g, 0.071 mL, 0.47 mmol), and the resulting mixture was treated with powdered (2S,3R,4S)-2-benzyl-3-isobutoxypentane-1,4-diol (0.10 g, 0.38 mmol). The vial was sealed with a screw cap and the mixture was warmed to 110° C. over a period of about 15 min, during which time the substrate melts, and stirred at 110° C. for an additional 15 min. The reaction mixture was cooled to room temperature and analyzed by thin layer chromatography (TLC; 2:1 hexanes in EtOAc developed with potassium permanganate (KMnO₄) or ceric ammonium molybdate) which indicated very little conversion. The reaction mixture was again heated to 110° C., stirred at 110° C. for 6 h, treated with additional 2-Bromo-1,1-diethoxyethane (0.028 g, 0.021 mL, 0.19 mmol), and warmed to and stirred at 120° C. for 4 h. The cooled reaction mixture was partitioned between H₂O and Et₂O and the phases were separated. The organic phase was dried over Na₂SO₄, filtered, concentrated, and the residue was purified by column chromatography (SiO₂; 10→50 EtOAc in hexanes) to give the title compound (72.7 mg, 50%) as a colorless oil: ¹H NMR (400 MHz, CDCl₃) δ 7.45-7.04 (m, 5H), 4.60 (t, J=5.3 Hz, 1H), 3.90 (td, J=6.5, 4.5 Hz, 1H), 3.69 (dqd, J=9.3, 7.0, 0.8 Hz, 2H), 3.62-3.47 (m, 3H), 3.46-3.36 (m, 2H), 3.34 (dd, J=9.5, 6.2 Hz, 1H), 3.24 (d, J=6.5 Hz, 2H), 3.18 (dd, J=6.8, 3.5 Hz, 1H), 2.98 (dd, J=13.7, 5.1 Hz, 1H), 2.88 (d, J=4.8 Hz, 1H), 2.67 (dd, J=13.7, 10.3 Hz, 1H), 2.13 (tdd, J=7.1, 3.4, 2.1 Hz, 1H), 1.82 (dp, J=13.2, 6.6 Hz, 1H), 1.26 (d, J=6.3 Hz, 3H), 1.21 (td, J=7.1, 3.8 Hz, 6H), 0.91 (dd, J=6.7, 1.2 Hz, 6H); ¹³C NMR (101 MHz, CDCl₃) δ 141.17, 129.12, 128.29, 125.84, 100.81, 83.93, 78.2, 71.4, 69.65, 68.63, 62.34, 62.3, 42.98, 34.41, 29.02, 19.74, 19.56, 19.5, 15.37, 15.29; ESIMS m/z 405 ([M+Na]⁺).

### Example 10, Step 2: Preparation of 2-(((2S,3R,4S)-2-benzyl-4-hydroxy-3-isobutoxypentyl)oxy)acetaldehyde

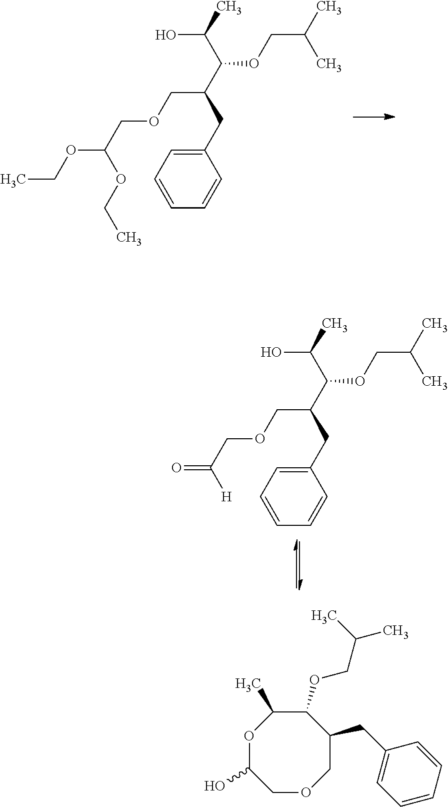 [SMILES: CCOC(COC[C@H](Cc1ccccc1)[C@@H](OCC(C)C)[C@H](C)O)OCC ; CC(C)CO[C@H]([C@H](COCC=O)Cc1ccccc1)[C@H](C)O ; CC(C)CO[C@@H]1[C@@H](Cc2ccccc2)COCC(O)O[C@H]1C]

To a solution of (2S,3R,4S)-4-benzyl-5-(2,2-diethoxyethoxy)-3-isobutoxypentan-2-ol (1.0 g, 2.6 mmol) in acetone (26 mL) was added 6 N HCl (5 mL), and the mixture was allowed to stir at room temperature for 24 h. The reaction was neutralized with sat'd aq NaHCO₃ solution, extracted with EtOAc (2×), and the combined organic extracts were dried over Na₂SO₄, filtered, and concentrated to give the title compound (0.824 g, 100%) as an apparent mixture of the desired aldehyde and diastereomeric hemi-acetals: ¹H NMR (400 MHz, CDCl₃) major aldehyde form δ 9.71-9.60 (m, 1H), 7.34-7.12 (m, 5H), 3.98 (dd, J=2.8, 0.8 Hz, 2H), 3.57-3.15 (m, 6H), 3.08 (dd, J=13.9, 4.7 Hz, 1H), 2.59 (dd, J=13.9, 10.5 Hz, 1H), 2.39 (d, J=4.2 Hz, 1H), 2.32-2.11 (m, 1H), 1.84 (dt, J=13.3, 6.7 Hz, 1H), 1.29 (d, J=6.4 Hz, 3H), 0.94 (dd, J=6.7, 3.2 Hz, 6H); ¹³C NMR (101 MHz, CDCl₃) δ 200.16, 140.89, 129.02, 128.39, 125.98, 82.81, 78.76, 76.37, 70.94, 68.37, 42.45, 33.48, 29.12, 19.56, 19.5; ESIMS m/z 309 ([M+H]⁺); α=0.748, [α]=22.0 (3.4 g/100 mL, CHCl₃).

### Example 10, Step 3: Preparation of (Z)-methyl 4-(((2S,3R,4S)-2-benzyl-4-hydroxy-3-isobutoxypentyl)oxy)-2-((tert-butoxycarbonyl)amino)but-2-enoate

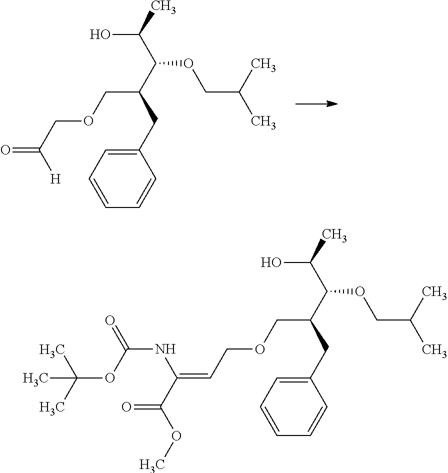 [SMILES: CC(C)CO[C@H]([C@H](COCC=O)Cc1ccccc1)[C@H](C)O ; COC(=O)/C(=C/COC[C@H](Cc1ccccc1)[C@@H](OCC(C)C)[C@H](C)O)NC(=O)OC(C)(C)C]

To a solution of 2-(((2S,3R,4S)-2-benzyl-4-hydroxy-3-isobutoxypentyl)oxy)-acetaldehyde (1.1 g, 3.6 mmol) and methyl 2-((tert-butoxycarbonyl)amino)-2-(dimethoxy-phosphoryl)acetate (1.06 g, 3.6 mmol) in CH₂Cl₂ (8 mL) was added DBU (0.57 g, 0.56 mL, 3.7 mmol) at −20° C. under N₂, and the reaction mixture was stirred while slowly warming to room temperature overnight. The mixture was diluted with EtOAc, washed with sat'd aq NH₄Cl solution, and the phases were separated. The organic phase was dried over Na₂SO₄, filtered, concentrated, and the residue was purified by column chromatography (SiO₂; 20% EtOAc in hexanes) to give the title compound (1.6 g, 94%) as a colorless oil: ¹H NMR (400 MHz, CDCl₃) δ 7.28 (m, 2H), 7.19 (m, 3H), 6.46 (s, 1H), 6.44 (s, 1H), 4.13-3.99 (m, 2H), 3.96-3.89 (m, 1H), 3.8 (s, 3H), 3.42-3.23 (m, 5H), 3.02 (dd, J=13.8, 4.8 Hz, 1H), 2.62-2.55 (m, 2H), 2.17 (d, J=3.9 Hz, 1H), 1.84 (dp, J=13.3, 6.7 Hz, 1H), 1.45 (s, 9H), 1.27 (d, J=6.3 Hz, 3H), 0.93 (dd, J=6.7, 2.3 Hz, 6H); ¹³C NMR (101 MHz, CDCl₃) δ 164.84, 152.95, 141.05, 129.08, 128.32, 125.86, 83.33, 80.99, 78.61, 69.54, 68.49, 67.91, 52.59, 42.74, 33.92, 29.06, 28.13, 19.55, 19.48. ESIMS m/z 478 ([M−H]⁻); α=0.633, [α]=22.21 (2.85 g/mL, CHCl₃).

### Example 10, Step 4: Preparation of (S)-methyl 4-(((2S,3R,4S)-2-benzyl-4-hydroxy-3-isobutoxypentyl)oxy)-2-((tert-butoxycarbonyl)amino)butanoate

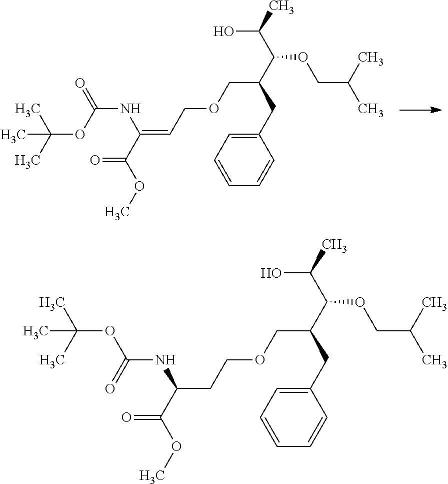

A solution of (Z)-methyl 4-(((2S,3R,4S)-2-benzyl-4-hydroxy-3-isobutoxypentyl)oxy)-2-((tert-butoxycarbonyl)amino)but-2-enoate (1.16 g, 2.42 mmol) in THF (10 mL) was transferred to a Parr shaker bottle and N₂ was bubbled through the solution for 10 min. The solution was treated with (+)-1,2-bis[(2S,5S)-2,5-diethylphospholano]benzene(cyclooctadiene)rhodium(I) tetrafluoroborate, ((S,S)-Et-DuPHOS-Rh; 0.080 g, 0.12 mmol) and the Parr bottle was evacuated under vacuum and backfilled with H₂ (3×). The mixture was placed under an H₂ atmosphere (45 psi) for 5 h, concentrated under reduced pressure, and the residue purified by column chromatography (SiO₂; 33% EtOAc in hexanes) to give a 9:1 mixture of diastereomers of the title compound (0.63 g, 54%) as a colorless oil: ¹H NMR (400 MHz, CDCl₃) δ 7.31-7.24 (m, 2H), 7.19 (m, 3H), 5.51 (d, J=7.8 Hz, 1H), 4.40 (q, J=6.5 Hz, 1H), 3.98-3.90 (m, 1H), 3.74 (s, 3H), 3.48-3.22 (m, 7H), 3.05 (dd, J=13.7, 4.3 Hz, 1H), 2.58-2.48 (m, 1H), 2.40 (d, J=4.8 Hz, 1H), 2.18-2.02 (m, 2H), 2.01-1.91 (m, 1H), 1.86 (dp, J=13.3, 6.7 Hz, 1H), 1.44 (s, 9H), 1.28 (d, J=6.3 Hz, 3H), 0.94 (dd, J=6.7, 3.5 Hz, 6H); ¹³C NMR (101 MHz, CDCl₃) δ 172.89, 155.43, 141.16, 129.13, 128.30, 125.83, 83.05, 79.91, 79.00, 70.06, 68.48, 67.44, 52.25, 51.90, 42.60, 33.56, 31.75, 29.15, 28.35, 19.59, 19.51, 19.21; ESIMS m/z 480 ([M−H]⁻); α=0.124, [α]=11.81 (1.05 g/mL, CHCl₃).

### Example 10, Step 5: Preparation of (S)-4-(((2S,3R,4S)-2-benzyl-4-hydroxy-3-isobutoxypentyl)oxy)-2-((tert-butoxycarbonyl)amino)butanoic acid

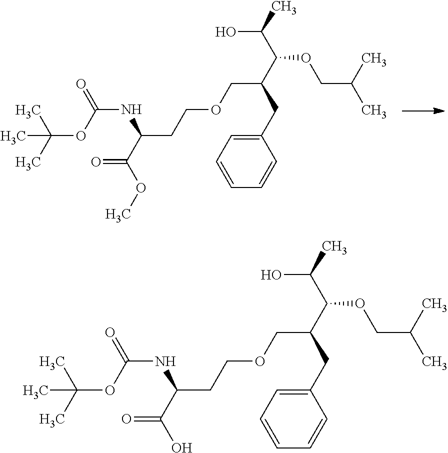

To a stirred solution of (S)-methyl 4-(((2S,3R,4S)-2-benzyl-4-hydroxy-3-isobutoxypentyl)oxy)-2-((tert-butoxycarbonyl)amino)butanoate (0.61 g, 1.27 mmol) in THF (12 mL) were added H₂O (6 mL) followed by LiOH.H₂O (0.16 g, 3.8 mmol), and the reaction mixture was stirred overnight at room temperature. The mixture was partitioned between EtOAc, brine, and 4 mL of 1 N HCl and the phases were separated. The aq phase was extracted with EtOAc, and the combined organic phases were dried over Na₂SO₄, filtered, and concentrated to give a 9:1 mixture of diastereomers of the title compound (0.584 g, 99%) as a colorless oil: ¹H NMR (400 MHz, CDCl₃) δ 7.31-7.24 (m, 2H), 7.19 (m, 3H), 6.58 (bs, 2H, COOH, OH), 5.60 (d, J=7.5 Hz, 1H), 4.39 (q, J=5.3 Hz, 1H), 4.00-3.87 (m, 1H), 3.49-3.35 (m, 3H), 3.27 (dd, J=8.7, 6.6 Hz, 4H), 3.02 (dd, J=13.9, 4.1 Hz, 1H), 2.51 (dd, J=13.6, 10.8 Hz, 1H), 2.18-2.03 (m, 3H), 1.85 (dp, J=13.2, 6.6 Hz, 1H), 1.44 (s, 9H), 1.27 (d, J=6.4 Hz, 3H), 0.93 (dd, J=6.7, 4.0 Hz, 6H); ¹³C NMR (101 MHz, CDCl₃) δ 175.73, 155.68, 141.03, 129.12, 128.32, 125.86, 82.97, 80.13, 79.21, 70.04, 68.74, 67.24, 51.77, 42.64, 33.48, 31.66, 29.13, 28.34, 19.58, 19.50, 19.10; ESIMS m/z 466 ([M−H]⁻); α=0.149, [α]=13.93 (1.07 g/mL, CHCl₃).

### Example 10, Step 6: Preparation of tert-butyl ((3S,8S,9R,10S)-8-benzyl-9-isobutoxy-10-methyl-2-oxo-1,6-dioxecan-3-yl)carbamate (Cmpd 222)

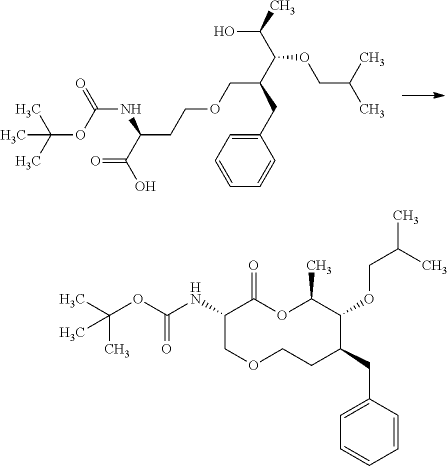

The title compound was prepared from (S)-4-(((2S,3R,4S)-2-benzyl-4-hydroxy-3-isobutoxypentyl)oxy)-2-((tert-butoxycarbonyl)amino)butanoic acid according to the methodology outlined in Example 1, Step 5 and was isolated as a colorless oil in 15% yield: ¹H NMR (400 MHz, CDCl₃) δ 7.31-7.25 (m, 2H), 7.22-7.14 (m, 3H), 5.50 (d, J=5.8 Hz, 1H), 5.04 (dq, J=9.5, 6.4 Hz, 1H), 4.29 (d, J=5.8 Hz, 1H), 3.67 (t, J=11.3 Hz, 1H), 3.43 (dd, J=8.3, 6.5 Hz, 1H), 3.34-3.19 (m, 4H), 3.17-3.11 (m, 1H), 3.03 (t, J'² 9.3 Hz, 1H), 2.50-2.39 (m, 1H), 2.14-1.98 (m, 2H), 1.88 (dt, J=13.2, 6.7 Hz, 1H), 1.76-1.64 (m, 1H), 1.43 (s, 9H), 1.39 (d, J=6.4 Hz, 3H), 0.95 (dd, J=6.7, 1.8 Hz, 6H); ESIMS m/z 472 [(M+Na)+].

### Example 11, Step 1: Preparation of (3S,8S,9R,10S)-3-amino-9-isobutoxy-10-methyl-8-propyloxecan-2-one hydrochloride (Cmpd 154)

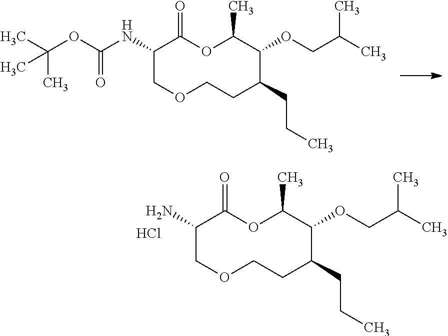 [SMILES: CCC[C@H]1CCOC[C@H](NC(=O)OC(C)(C)C)C(=O)O[C@@H](C)[C@@H]1OCC(C)C ; Cl ; CCC[C@H]1CCOC[C@H](N)C(=O)O[C@@H](C)[C@@H]1OCC(C)C]

To a vial containing tert-butyl ((3S,8S,9R,10S)-9-isobutoxy-10-methyl-2-oxo-8-propyloxecan-3-yl)carbamate (130 mg, 0.325 mmol) was added a solution of HCl in dioxane (1.63 mL, 6.51, 4 M) mmol) and the resulting solution was stirred at room temperature for 30 min. The reaction mixture was concentrated to provide the title compound (109 mg, 100%) as a white solid: ESIMS m/z 300.4 ([M+H]⁺).

### Example 11, Step 2: Preparation of 3-hydroxy-N-((3S,8S,9R,10S)-9-isobutoxy-10-methyl-2-oxo-8-propyloxecan-3-yl)-4-methoxypicolinamide (Cmpd 105)

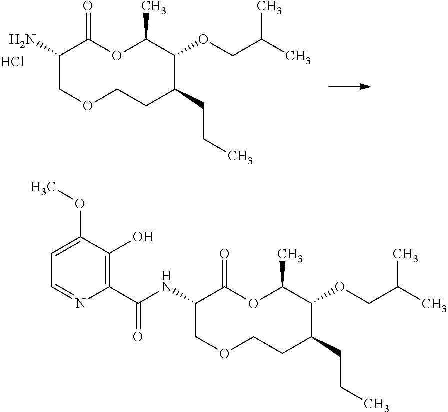 [SMILES: Cl ; CCC[C@H]1CCOC[C@H](N)C(=O)O[C@@H](C)[C@@H]1OCC(C)C ; CCC[C@H]1CCOC[C@H](NC(=O)c2nccc(OC)c2O)C(=O)O[C@@H](C)[C@@H]1OCC(C)C]

To a solution of (3S,8S,9R,10S)-3-amino-9-isobutoxy-10-methyl-8-propyloxecan-2-one hydrochloride (109 mg, 0.325 mmol) in anhydrous CH₂Cl₂ (3.3 mL) were added 3-hydroxy-4-methoxypicolinic acid (60.5 mg, 0.358 mmol), PyBOP (186 mg, 0.358 mmol), and ethyldiisopropyl amine (187 μL, 1.07 mmol), and the resulting homogeneous pink solution was stirred at room temperature for 2.5 h. The resulting yellow solution was concentrated and purified by column chromatography (SiO₂, 5→50% acetone in hexanes) to provide a mixture of product and acetone hydrate, the latter of which was removed by azeotropic distillation with toluene to provide the title compound (115 mg, 78%) as a white solid: ¹H NMR (400 MHz, CDCl₃) δ 12.17 (s, 1H), 8.71 (d, J=7.4 Hz, 1H), 7.99 (dd, J=5.2, 1.5 Hz, 1H), 6.87 (d, J=5.1 Hz, 1H), 4.95 (dq, J=12.8, 6.3 Hz, 1H), 4.74 (dt, J=7.6, 4.1 Hz, 1H), 3.93 (s, 3H), 3.32 (p, J=8.1 Hz, 2H), 2.95 (t, J=9.2 Hz, 1H), 2.30-2.15 (m, 1H), 2.15-1.96 (m, 1H), 1.84 (dp, J=13.1, 6.5 Hz, 1H), 1.64 (tt, J=10.0, 4.4 Hz, 1H), 1.59-1.48 (m, 4H), 1.48-1.31 (m, 5H), 1.31-1.07 (m, 4H), 0.99-0.84 (m, 9H); ¹³C NMR (101 MHz, CDCl₃) δ 171.87, 168.69, 155.28, 148.63, 140.53, 130.51, 128.98, 128.17, 125.25, 109.41, 84.55, 79.49, 75.95, 56.02, 51.48, 43.09, 34.33, 29.10, 27.32, 26.84, 24.90, 21.86, 20.17, 19.52, 19.49, 18.48, 14.43; HRMS-ESI (m/z) [M+H]⁺ calcd for C₂₄H₃₉N₂O₆, 451.2803; found, 451.2809.

### Example 11, Step 3A: Preparation of ((2-(((3S,8S,9R,10S)-9-isobutoxy-10-methyl-2-oxo-8-propyloxecan-3-yl)carbamoyl)-4-methoxypyridin-3-yl)oxy)methyl acetate (Cmpd 30)

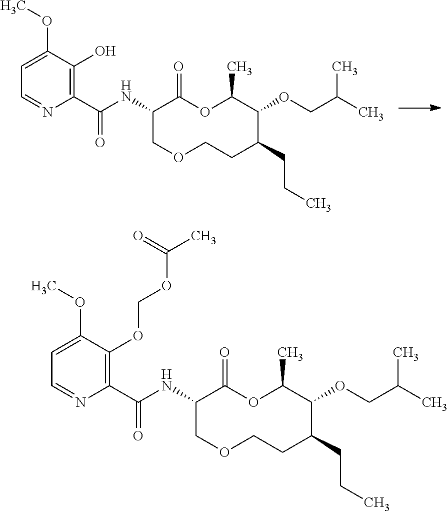 [SMILES: CCC[C@H]1CCOC[C@H](NC(=O)c2nccc(OC)c2O)C(=O)O[C@@H](C)[C@@H]1OCC(C)C ; CCC[C@H]1CCOC[C@H](NC(=O)c2nccc(OC)c2OCOC(C)=O)C(=O)O[C@@H](C)[C@@H]1OCC(C)C]

To a solution of 3-hydroxy-N-((3S,8S,9R,10S)-9-isobutoxy-10-methyl-2-oxo-8-propyloxecan-3-yl)-4-methoxypicolinamide (90.4 mg, 0.201 mmol) in anhydrous acetone (2 mL) were added powdered K₂CO₃ (55.5 mg, 0.401 mmol) and bromomethyl acetate (46.0 mg, 0.301 mmol), and the resulting mixture was warmed to and stirred vigorously at 50° C. overnight. The reaction mixture was cooled to room temperature, concentrated, and purified by column chromatography (SiO₂, 5→50% acetone in hexanes) to provide the title compound (81 mg, 77%) as a light-yellow solid: ¹H NMR (400 MHz, CDCl₃) δ 8.55 (d, J=7.3 Hz, 1H), 8.29 (d, J=5.4 Hz, 1H), 6.96 (d, J=5.4 Hz, 1H), 5.75 (q, J=6.4 Hz, 2H), 4.93 (dq, J=9.2, 6.3 Hz, 1H), 4.76 (ddd, J=7.6, 5.2, 3.5 Hz, 1H), 3.91 (s, 3H), 3.44-3.20 (m, 2H), 2.95 (t, J=9.3 Hz, 1H), 2.28-2.13 (m, 1H), 2.13-1.97 (m, 4H), 1.94-1.75 (m, 1H), 1.72-1.58 (m, 1H), 1.58-1.46 (m, 4H), 1.46-1.31 (m, 5H), 1.32-1.06 (m, 4H), 0.91 (dd, J=15.4, 7.0 Hz, 9H); ¹³C NMR (101 MHz, CDCl₃) δ 172.53, 170.20, 163.01, 160.24, 145.80, 143.83, 142.60, 109.54, 89.53, 84.56, 79.45, 75.71, 56.17, 51.72, 43.14, 34.31, 29.09, 27.28, 26.92, 24.91, 21.96, 20.84, 20.14, 19.52, 19.49, 18.48, 14.42; HRMS-ESI (m/z) [M+H]⁺ calcd for C₂₇H₄₃N₂O₈, 523.3014; found, 523.3027.

### Example 11, Step 3B: Preparation of (2S,3R,4R,9S)-9-(3-acetoxy-4-methoxypicolinamido)-4-(cyclopentylmethyl)-2-methyl-10-oxooxecan-3-yl isobutyrate (Cmpd 56)

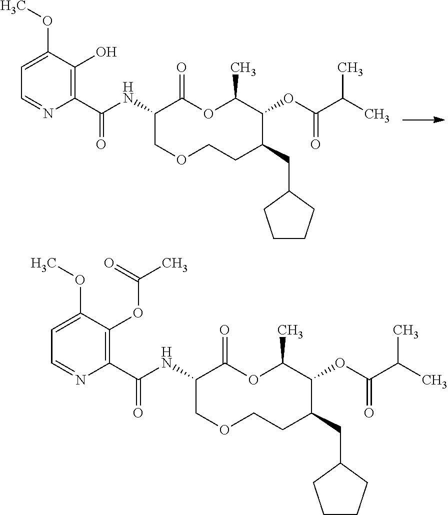 [SMILES: COc1ccnc(C(=O)N[C@H]2COCC[C@H](CC3CCCC3)[C@@H](OC(=O)C(C)C)[C@H](C)OC2=O)c1O ; COc1ccnc(C(=O)N[C@H]2COCC[C@H](CC3CCCC3)[C@@H](OC(=O)C(C)C)[C@H](C)OC2=O)c1OC(C)=O]

To a solution of (2S,3R,4R,9S)-4-(cyclopentylmethyl)-9-(3-hydroxy-4-methoxypicolinamido)-2-methyl-10-oxooxecan-3-yl isobutyrate (102 mg, 0.202 mmol) in anhydrous CH₂Cl₂ (2 mL) were added NEt₃ (56.3 μL, 0.404 mmol), DMAP (4.94 mg, 0.040 mmol), and acetyl chloride (21 μL, 0.30 mmol), and the resulting yellow solution was stirred overnight at room temperature. The reaction mixture was concentrated and purified by column chromatography (SiO₂, 5→50% acetone in hexanes) to provide the title compound (109 mg, 99%) as a white solid: ¹H NMR (400 MHz, CDCl₃) δ 8.70 (s, 1H), 8.36 (d, J=5.4 Hz, 1H), 7.01 (d, J=5.5 Hz, 1H), 5.11-4.99 (m, 1H), 4.82 (t, J=9.5 Hz, 1H), 4.77 (ddd, J=8.0, 5.3, 3.5 Hz, 1H), 3.91 (s, 3H), 2.58 (hept, J=7.0 Hz, 1H), 2.40 (s, 3H), 2.26-2.15 (m, 1H), 2.04 (d, J=5.2 Hz, 1H), 1.91-1.79 (m, 1H), 1.79-1.63 (m, 3H), 1.64-1.59 (m, 2H), 1.57-1.44 (m, 6H), 1.34-1.25 (m, 1H), 1.24-1.12 (m, 12H), 1.07-0.95 (m, 2H); ¹³C NMR (101 MHz, CDCl₃) δ 176.23, 172.53, 168.93, 162.45, 159.43, 146.74, 141.60, 137.46, 109.74, 73.84, 56.29, 51.50, 40.86, 38.45, 37.17, 34.32, 33.69, 32.10, 27.21, 26.97, 25.09, 25.02, 24.58, 21.90, 20.77, 19.07, 19.02, 17.97; HRMS-ESI (m/z) [M+H]⁺ calcd for C₂₉H₄₃N₂O₈, 547.3014; found, 547.3028.

### Example 11, Step 3C: Preparation of ((2-(((3S,8R,9R,10S)-8-(cyclopentylmethyl)-10-methyl-2-oxo-9-phenoxyoxecan-3-yl)carbamoyl)-4-methoxypyridin-3-yl)oxy)methyl 2-ethoxyacetate (Cmpd 51)

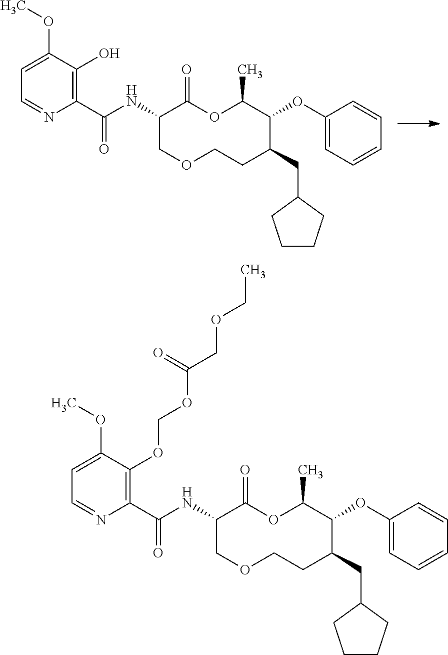

To a solution of N-((3S,8R,9R,10S)-8-(cyclopentylmethyl)-10-methyl-2-oxo-9-phenoxyoxecan-3-yl)-3-hydroxy-4-methoxypicolinamide (110 mg, 0.215 mmol) in anhydrous acetone (2.2 mL) were added K₂CO₃ (59.5 mg, 0.431 mmol), NaI (6.5 mg, 0.043 mmol), and chloromethyl 2-ethoxyacetate (49.3 mg, 0.323 mmol), and the resulting mixture was warmed to and vigorously stirred at 50° C. overnight. The reaction was cooled to room temperature, concentrated, and purified by column chromatography (SiO₂, 5→50% acetone in hexanes) to provide the title compound (85 mg, 63%) as a white solid: ¹H NMR (400 MHz, CDCl₃) δ 8.57 (d, J=7.3 Hz, 1H), 8.30 (d, J=5.4 Hz, 1H), 7.37-7.21 (m, 2H), 7.04-6.83 (m, 4H), 5.91-5.73 (m, 2H), 5.22-5.08 (m, 1H), 4.87-4.70 (m, 1H), 4.18-4.06 (m, 3H), 3.91 (s, 3H), 3.59 (q, J=7.0 Hz, 2H), 2.29-2.15 (m, 1H), 2.15-2.02 (m, 1H), 1.88 (dd, J=14.2, 6.6 Hz, 1H), 1.75-1.60 (m, 5H), 1.58-1.41 (m, 7H), 1.30 (s, 2H), 1.28 (d, J=6.3 Hz, 3H), 1.23 (t, J=7.0 Hz, 3H), 1.18 (dt, J=9.4, 4.7 Hz, 1H), 1.04-0.93 (m, 2H); ¹³C NMR (101 MHz, CDCl₃) δ 172.51, 170.06, 163.00, 160.19, 159.64, 145.88, 143.88, 142.46, 129.57, 120.82, 115.42, 109.66, 89.59, 82.47, 75.33, 67.80, 67.20, 56.22, 51.90, 42.13, 38.66, 37.54, 33.57, 32.09, 27.35, 25.10, 25.04, 24.59, 21.91, 18.73, 15.02; HRMS-ESI (m/z) [M+H]⁺ calcd for C₃₄H₄₇N₂O₉, 627.3276; found, 627.3285.

### Example 11, Step 3D: Preparation of ((4-methoxy-2-(((3S,8S,9R,10S)-8-(4-methoxybenzyl)-10-methyl-2-oxo-9-phenoxy-1,5-dioxecan-3-yl)carbamoyl)pyridin-3-yl)oxy)methyl isobutyrate (Cmpd 67)

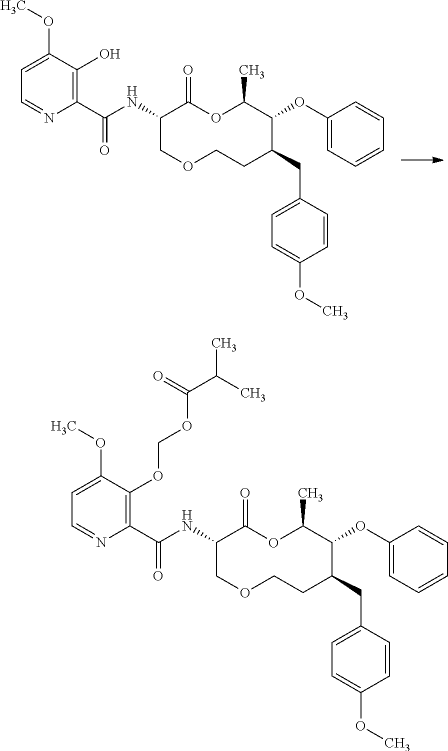 [SMILES: COc1ccc(C[C@H]2CCOC[C@H](NC(=O)c3nccc(OC)c3O)C(=O)O[C@@H](C)[C@@H]2Oc2ccccc2)cc1 ; COc1ccc(C[C@H]2CCOC[C@H](NC(=O)c3nccc(OC)c3OCOC(=O)C(C)C)C(=O)O[C@@H](C)[C@@H]2Oc2ccccc2)cc1]

To a solution of 3-hydroxy-4-methoxy-N-((3S,8S,9R,10S)-8-(4-methoxybenzyl)-10-methyl-2-oxo-9-phenoxy-1,5-dioxecan-3-yl)picolinamide (87 mg, 0.16 mmol) in anhydrous acetone (2 mL) were added K₂CO₃ (43.7 mg, 0.316 mmol), NaI (2.4 mg, 0.016 mmol), and chloromethyl isobutyrate (32.4 mg, 0.237 mmol), and the reaction mixture was warmed to and vigorously stirred at 45° C. overnight. The mixture was cooled and concentrated, and the residue was purified by column chromatography (SiO₂, 5→50% acetone in hexanes) to provide the title compound (69.6 mg, 68%) of as a white solid: ¹H NMR (500 MHz, CDCl₃) δ 8.75 (d, J=8.0 Hz, 1H), 8.31 (d, J=5.4 Hz, 1H), 7.31 (tt, J=7.3, 2.2 Hz, 2H), 7.08-7.00 (m, 2H), 6.99-6.95 (m, 1H), 6.95-6.91 (m, 3H), 6.80-6.76 (m, 2H), 5.83-5.71 (m, 2H), 5.41 (dq, J=9.2, 6.2 Hz, 1H), 5.01-4.94 (m, 1H), 4.11 (dd, J=8.9, 7.9 Hz, 1H), 3.91-3.87 (m, 4H), 3.83 (dd, J=10.5, 1.1 Hz, 1H), 3.76 (s, 3H), 3.23-3.07 (m, 2H), 2.89-2.75 (m, 1H), 2.54 (hept, J=7.0 Hz, 1H), 2.28 (dd, J=10.5, 7.4 Hz, 1H), 2.14 (dd, J=13.6, 10.7 Hz, 1H), 2.09-1.97 (m, 1H), 1.48-1.39 (m, 1H), 1.33 (d, J=6.3 Hz, 3H), 1.14 (dd, J=7.0, 0.6 Hz, 6H); ¹³C NMR (126 MHz, CDCl₃) δ 176.21, 169.40, 163.39, 160.24, 159.23, 157.84, 145.74, 144.19, 142.08, 132.23, 129.99, 129.68, 121.12, 115.36, 113.77, 109.56, 89.95, 82.85, 74.23, 68.53, 68.17, 56.12, 55.18, 53.93, 39.00, 37.64, 33.86, 32.28, 18.98, 18.68; HRMS-ESI (m/z) [M+H]⁺ calcd for C₃₅H₄₃O₁₀N₂, 651.2912; found, 651.2924.

### Example A: Evaluation of Fungicidal Activity: Leaf Blotch of Wheat (Zymoseptoria Tritici; Bayer Code SEPTTR)

Technical grades of materials were dissolved in acetone, which were then mixed with nine volumes of H₂O containing 110 ppm Triton X-100. The fungicide solutions were applied onto wheat seedlings using an automated booth sprayer to run-off. All sprayed plants were allowed to air dry prior to further handling. All fungicides were evaluated using the aforementioned method for their activity vs. all target diseases, unless stated otherwise. Wheat leaf blotch and brown rust activity were also evaluated using track spray applications, in which case the fungicides were formulated as EC formulations, containing 0.1% Trycol 5941 in the spray solutions.

Wheat plants (variety Yuma) were grown from seed in a greenhouse in 50% mineral soil/50% soil-less Metro mix until the first leaf was fully emerged, with 7-10 seedlings per pot. These plants were inoculated with an aqueous spore suspension of Zymoseptoria tritici either prior to or after fungicide treatments. After inoculation the plants were kept in 100% relative humidity (one day in a dark dew chamber followed by two to three days in a lighted dew chamber at 20° C.) to permit spores to germinate and infect the leaf. The plants were then transferred to a greenhouse set at 20° C. for disease to develop. When disease symptoms were fully expressed on the 1^{st} leaves of untreated plants, infection levels were assessed on a scale of 0 to 100 percent disease severity. Percent disease control was calculated using the ratio of disease severity on treated plants relative to untreated plants.

### Example B: Evaluation of Fungicidal Activity: Wheat Brown Rust (Puccinia triticina; Bayer Code PUCCRT)

Wheat plants (variety Yuma) were grown from seed in a greenhouse in 50% mineral soil/50% soil-less Metro mix until the first leaf was fully emerged, with 7-10 seedlings per pot. These plants were inoculated with an aqueous spore suspension of Puccinia triticina either prior to or after fungicide treatments. After inoculation the plants were kept in a dark dew room at 22° C. with 100% relative humidity overnight to permit spores to germinate and infect the leaf. The plants were then transferred to a greenhouse set at 24° C. for disease to develop. Fungicide formulation, application and disease assessment followed the procedures as described in the Example A.

### Example C: Evaluation of Fungicidal Activity: Wheat Glume Blotch (Leptosphaeria nodorum; Bayer Code LEPTNO)

Wheat plants (variety Yuma) were grown from seed in a greenhouse in 50% mineral soil/50% soil-less Metro mix until the first leaf was fully emerged, with 7-10 seedlings per pot. These plants were inoculated with an aqueous spore suspension of Leptosphaeria nodorum 24 hr after fungicide treatments. After inoculation the plants were kept in 100% relative humidity (one day in a dark dew chamber followed by two days in a lighted dew chamber at 20° C.) to permit spores to germinate and infect the leaf. The plants were then transferred to a greenhouse set at 20° C. for disease to develop. Fungicide formulation, application and disease assessment followed the procedures as described in the Example A.

### Example D: Evaluation of Fungicidal Activity: Apple Scab (Venturia inaequalis; Bayer Code VENTIN)

Apple seedlings (variety McIntosh) were grown in soil-less Metro mix, with one plant per pot. Seedlings with two expanding young leaves at the top (older leaves at bottom of the plants were trimmed) were used in the test. Plants were inoculated with a spore suspension of Venturia inaequalis 24 hr after fungicide treatment and kept in a 22° C. dew chamber with 100% relative humidity for 48 hr, and then moved to a greenhouse set at 20° C. for disease to develop. Fungicide formulation, application and disease assessment on the sprayed leaves followed the procedures as described in the Example A.

### Example E: Evaluation of Fungicidal Activity: Grape Powdery Mildew (Uncinula Necator; Bayer Code UNCINE)

Grape seedlings (variety Carignane) were grown in soil-less Metro mix, with one plant per pot, and used in the test when approximately one month old. Plants were inoculated 24 hr after fungicide treatment by shaking spores from infected leaves over test plants. Plants were maintained in a greenhouse set at 20° C. until disease was fully developed. Fungicide formulation, application and disease assessment on the sprayed leaves followed the procedures as described in the Example A.

### Example F: Evaluation of Fungicidal Activity: Powdery Mildew of Cucumber (Erysiphe cichoracearum; Bayer Code ERYSCI)

Cucumber seedlings (variety Bush Pickle) were grown in soil-less Metro mix, with one plant per pot, and used in the test when 12 to 14 days old. Plants were inoculated with a spore suspension 24 hr following fungicide treatments. After inoculation the plants remained in the greenhouse set at 20° C. until disease was fully expressed. Fungicide formulation, application and disease assessment on the sprayed leaves followed the procedures as described in the Example A.

### Example G: Evaluation of Fungicidal Activity: Leaf Spot of Sugar Beets (Cercospora Beticola; Bayer Code CERCBE)

Sugar beet plants (variety HH88) were grown in soil-less Metro mix and trimmed regularly to maintain a uniform plant size prior to test. Plants were inoculated with a spore suspension 24 hr after fungicide treatments. Inoculated plants were kept in a dew chamber at 22° C. for 48 hr then incubated in a greenhouse set at 24° C. under a clear plastic hood with bottom ventilation until disease symptoms were fully expressed. Fungicide formulation, application and disease assessment on the sprayed leaves followed the procedures as described in the Example A.

### Example H: Evaluation of Fungicidal Activity: Asian Soybean Rust (Phakopsora Pachyrhizi; Bayer Code PHAKPA)

Technical grades of materials were dissolved in acetone, which were then mixed with nine volumes of H₂O containing 0.011% Tween 20. The fungicide solutions were applied onto soybean seedlings using an automated booth sprayer to run-off. All sprayed plants were allowed to air dry prior to further handling.

Soybean plants (variety Williams 82) were grown in soil-less Metro mix, with one plant per pot. Two weeks old seedlings were used for testing. Plants were inoculated either 3 days prior to or 1 day after fungicide treatments. Plants were incubated for 24 h in a dark dew room at 22° C. and 100% relative humidity then transferred to a growth room at 23° C. for disease to develop. Disease severity was assessed on the sprayed leaves.

### Example I: Evaluation of Fungicidal Activity: Wheat Powdery Mildew (Blumeria Graminis f.sp. Tritici; Synonym: Erysiphe graminis f.sp. Tritici; Bayer Code ERYSGT)

Wheat plants (variety Yuma) were grown from seed in a greenhouse in 50% mineral soil/50% soil-less Metro mix until the first leaf was fully emerged, with 7-10 seedlings per pot. These plants were inoculated by dusting with infected stock plants 24 hr after fungicide treatments. After inoculation the plants were kept in a greenhouse set at 20° C. for disease to develop. Fungicide formulation, application and disease assessment on the sprayed leaves followed the procedures as described in the Example A.

### Example J: Evaluation of Fungicidal Activity: Barley Powdery Mildew (Blumeria Graminis f.sp. Hordei; Synonym: Erysiphe graminis f.sp. Hordei; Bayer Code ERYSGH)

Barley seedlings (variety Harrington) were propagated in soil-less Metro mix, with each pot having 8 to 12 plants, and used in the test when first leaf was fully emerged. Test plants were inoculated by dusting with infected stock plants 24 hr after fungicide treatments. After inoculation the plants were kept in a greenhouse set at 20° C. for disease to develop. Fungicide formulation, application and disease assessment on the sprayed leaves followed the procedures as described in the Example A.

### Example K: Evaluation of Fungicidal Activity: Barley Scald (Rhyncosporium Secalis; Bayer Code RHYNSE)

Barley seedlings (variety Harrington) were propagated in soil-less Metro mix, with each pot having 8 to 12 plants, and used in the test when first leaf was fully emerged. Test plants were inoculated by an aqueous spore suspension of Rhyncosporium secalis 24 hr after fungicide treatments. After inoculation the plants were kept in a dew room at 20° C. with 100% relative humidity for 48 hr. The plants were then transferred to a greenhouse set at 20° C. for disease to develop. Fungicide formulation, application and disease assessment on the sprayed leaves followed the procedures as described in the Example A.

### Example L: Evaluation of Fungicidal Activity: Rice Blast (Magnaporthe grisea; Anamorph: Pyricularia Oryzae; Bayer Code PYRIOR)

Rice seedlings (variety Japonica) were propagated in soil-less Metro mix, with each pot having 8 to 14 plants, and used in the test when 12 to 14 days old. Test plants were inoculated with an aqueous spore suspension of Pyricularia oryzae 24 hr after fungicide treatments. After inoculation the plants were kept in a dew room at 22° C. with 100% relative humidity for 48 hr to permit spores to germinate and infect the leaf. The plants were then transferred to a greenhouse set at 24° C. for disease to develop. Fungicide formulation, application and disease assessment on the sprayed leaves followed the procedures as described in the Example A.

### Example M: Evaluation of Fungicidal Activity: Tomato Early Blight (Alternaria Solani; Bayer Code ALTESO)

Tomato plants (variety Outdoor Girl) were propagated in soil-less Metro mix, with each pot having one plant, and used when 12 to 14 days old. Test plants were inoculated with an aqueous spore suspension of Alternaria solani 24 hr after fungicide treatments. After inoculation the plants were kept in 100% relative humidity (one day in a dark dew chamber followed by two to three days in a lighted dew chamber at 20° C.) to permit spores to germinate and infect the leaf. The plants were then transferred to a growth room at 22° C. for disease to develop. Fungicide formulation, application and disease assessment on the sprayed leaves followed the procedures as described in the Example A.

### Example N: Evaluation of Fungicidal Activity: Cucumber Anthracnose (Glomerella Lagenarium; Anamorph: Colletotrichum lagenarium; Bayer Code COLLLA)

Cucumber seedlings (variety Bush Pickle) were propagated in soil-less Metro mix, with each pot having one plant, and used in the test when 12 to 14 days old. Test plants were inoculated with an aqueous spore suspension of Colletotrichum lagenarium 24 hr after fungicide treatments. After inoculation the plants were kept in a dew room at 22° C. with 100% relative humidity for 48 hr to permit spores to germinate and infect the leaf. The plants were then transferred to a growth room set at 22° C. for disease to develop. Fungicide formulation, application and disease assessment on the sprayed leaves followed the procedures as described in the Example A.

TABLE 1
Compound Structure, Appearance, and Preparation Method

| Com- | | Prepared | |
|---|---|---|---|
| pound | | According | |
| Num- | | to | |
| ber | Structure | Example: | Appearance |
| 1 | 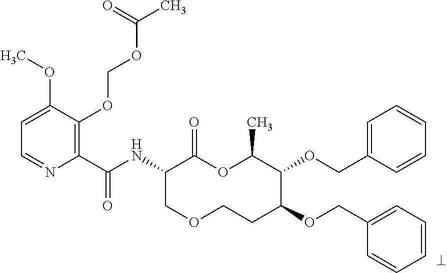 [SMILES: COc1ccnc(C(=O)N[C@H]2COCC[C@H](OCc3ccccc3)[C@@H](OCc3ccccc3)[C@H](C)OC2=O)c1OCOC(C)=O] | Example 11, Step 3A | Colorless Oil |
| 2 | 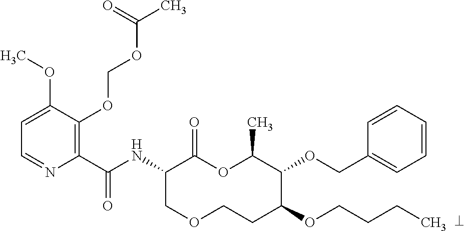 [SMILES: CCCCO[C@H]1CCOC[C@H](NC(=O)c2nccc(OC)c2OCOC(C)=O)C(=O)O[C@@H](C)[C@@H]1OCc1ccccc1] | Example 11, Step 3A | Glassy Solid |
| 3 | 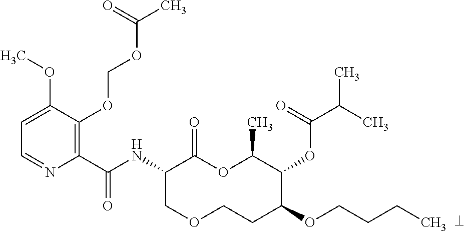 [SMILES: CCCCO[C@H]1CCOC[C@H](NC(=O)c2nccc(OC)c2OCOC(C)=O)C(=O)O[C@@H](C)[C@@H]1OC(=O)C(C)C] | Example 11, Step 3A | White Solid |
| 4 | 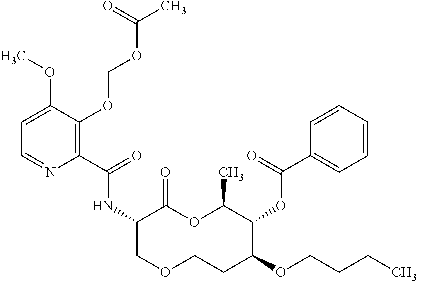 [SMILES: CCCCO[C@H]1CCOC[C@H](NC(=O)c2nccc(OC)c2OCOC(C)=O)C(=O)O[C@@H](C)[C@@H]1OC(=O)c1ccccc1] | Example 11, Step 3A | White Solid |
| 5 | 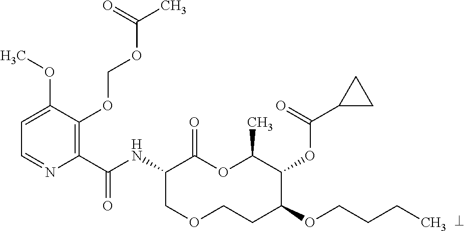 [SMILES: CCCCO[C@H]1CCOC[C@H](NC(=O)c2nccc(OC)c2OCOC(C)=O)C(=O)O[C@@H](C)[C@@H]1OC(=O)C1CC1] | Example 11, Step 3A | White Solid |
| 6 | 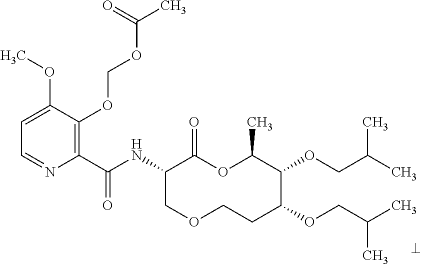 [SMILES: COc1ccnc(C(=O)N[C@H]2COCC[C@@H](OCC(C)C)[C@@H](OCC(C)C)[C@H](C)OC2=O)c1OCOC(C)=O] | Example 11, Step 3A | White Solid |
| 7 | 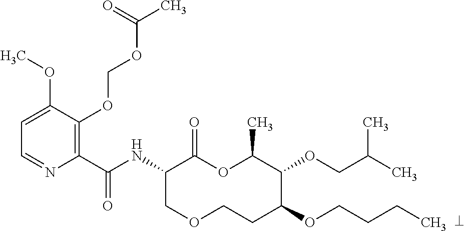 [SMILES: CCCCO[C@H]1CCOC[C@H](NC(=O)c2nccc(OC)c2OCOC(C)=O)C(=O)O[C@@H](C)[C@@H]1OCC(C)C] | Example 11, Step 3A | Gooey Solid |
| 8 | 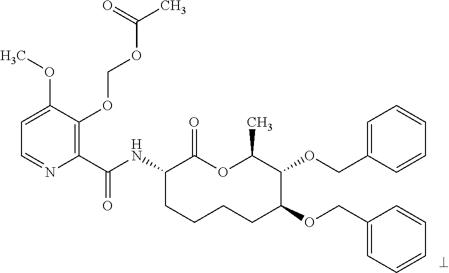 [SMILES: COc1ccnc(C(=O)N[C@H]2CCCC[C@H](OCc3ccccc3)[C@@H](OCc3ccccc3)[C@H](C)OC2=O)c1OCOC(C)=O] | Example 11, Step 3A | Light Yellow Oil |
| 9 | 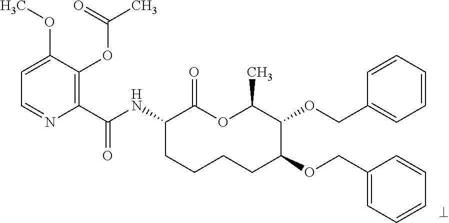 [SMILES: COc1ccnc(C(=O)N[C@H]2CCCC[C@H](OCc3ccccc3)[C@@H](OCc3ccccc3)[C@H](C)OC2=O)c1OC(C)=O] | Example 11, Step 3B | Sticky White Solid |
| 10 | 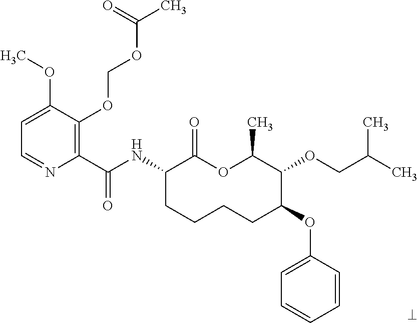 [SMILES: COc1ccnc(C(=O)N[C@H]2CCCC[C@H](Oc3ccccc3)[C@@H](OCC(C)C)[C@H](C)OC2=O)c1OCOC(C)=O] | Example 11, Step 3A | Clear Light Yellow Oil |
| 11 | 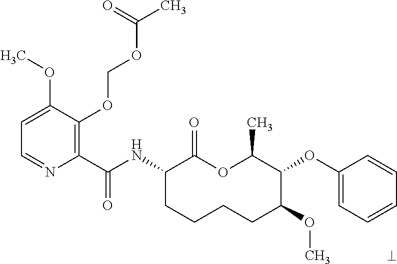 [SMILES: COc1ccnc(C(=O)N[C@H]2CCCC[C@H](OC)[C@@H](Oc3ccccc3)[C@H](C)OC2=O)c1OCOC(C)=O] | Example 11, Step 3A | Off White Clear Oil |
| 12 | 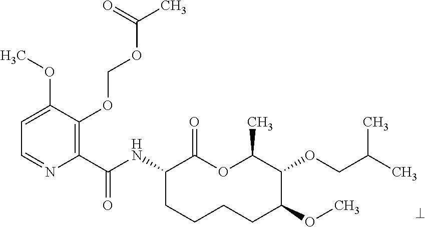 [SMILES: COc1ccnc(C(=O)N[C@H]2CCCC[C@H](OC)[C@@H](OCC(C)C)[C@H](C)OC2=O)c1OCOC(C)=O] | Example 11, Step 3A | Sticky Yellow Solid |
| 13 | 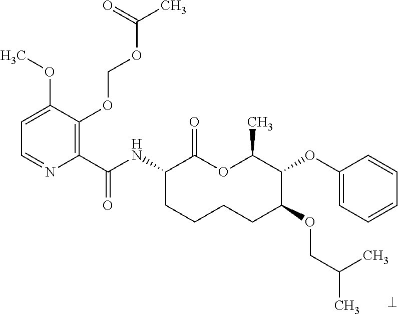 [SMILES: COc1ccnc(C(=O)N[C@H]2CCCC[C@H](OCC(C)C)[C@@H](Oc3ccccc3)[C@H](C)OC2=O)c1OCOC(C)=O] | Example 11, Step 3A | Clear Colorless Oil |
| 14 | 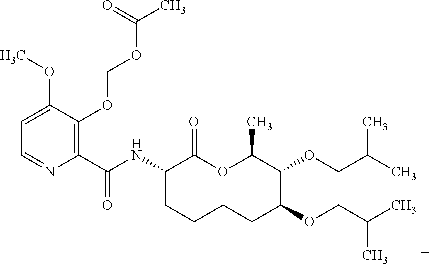 [SMILES: COc1ccnc(C(=O)N[C@H]2CCCC[C@H](OCC(C)C)[C@@H](OCC(C)C)[C@H](C)OC2=O)c1OCOC(C)=O] | Example 11, Step 3A | Clear Colorless Oil |
| 15 | 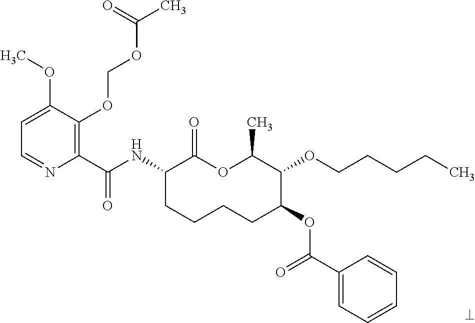 [SMILES: CCCCCO[C@H]1[C@H](C)OC(=O)[C@@H](NC(=O)c2nccc(OC)c2OCOC(C)=O)CCCC[C@@H]1OC(=O)c1ccccc1] | Example 11, Step 3A | Yellow Oil |
| 16 | 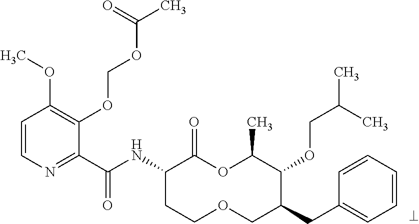 [SMILES: COc1ccnc(C(=O)N[C@H]2CCOC[C@H](Cc3ccccc3)[C@@H](OCC(C)C)[C@H](C)OC2=O)c1OCOC(C)=O] | Example 11, Step 3A | Off White Powder |
| 17 | 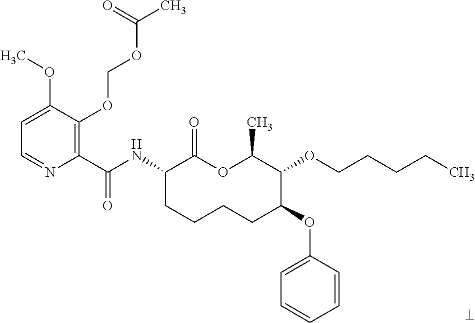 [SMILES: CCCCCO[C@H]1[C@H](C)OC(=O)[C@@H](NC(=O)c2nccc(OC)c2OCOC(C)=O)CCCC[C@@H]1Oc1ccccc1] | Example 11, Step 3A | Yellow Sticky Solid |
| 18 | 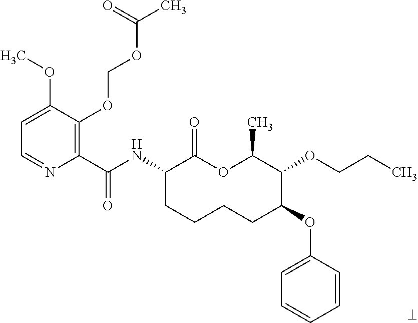 [SMILES: CCCO[C@H]1[C@H](C)OC(=O)[C@@H](NC(=O)c2nccc(OC)c2OCOC(C)=O)CCCC[C@@H]1Oc1ccccc1] | Example 11, Step 3A | Yellow Oil |
| 19 | 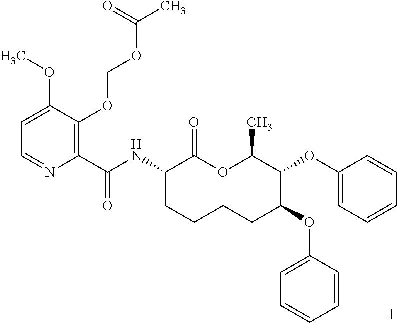 [SMILES: COc1ccnc(C(=O)N[C@H]2CCCC[C@H](Oc3ccccc3)[C@@H](Oc3ccccc3)[C@H](C)OC2=O)c1OCOC(C)=O] | Example 11, Step 3A | Clear Yellow Oil |
| 20 | 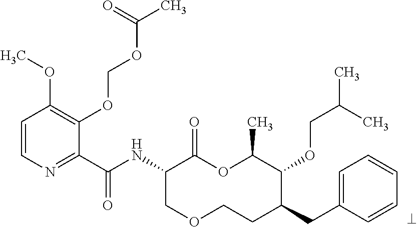 [SMILES: COc1ccnc(C(=O)N[C@H]2COCC[C@H](Cc3ccccc3)[C@@H](OCC(C)C)[C@H](C)OC2=O)c1OCOC(C)=O] | Example 11, Step 3A | Colorless Oil |
| 21 | 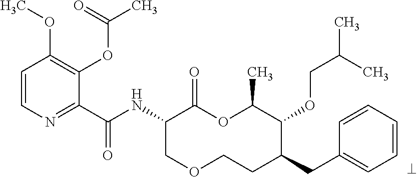 [SMILES: COc1ccnc(C(=O)N[C@H]2COCC[C@H](Cc3ccccc3)[C@@H](OCC(C)C)[C@H](C)OC2=O)c1OC(C)=O] | Example 11, Step 3B | Colorless Oil |
| 22 | 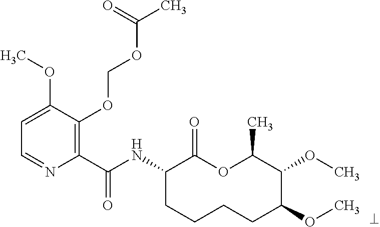 [SMILES: COc1ccnc(C(=O)N[C@H]2CCCC[C@H](OC)[C@@H](OC)[C@H](C)OC2=O)c1OCOC(C)=O] | Example 11, Step 3A | White Solid |
| 23 | 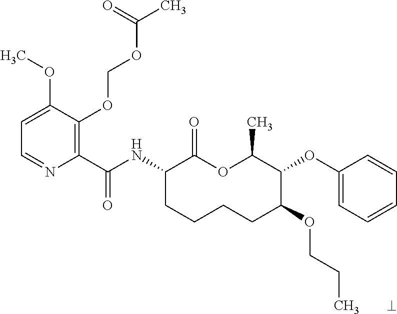 [SMILES: CCCO[C@H]1CCCC[C@H](NC(=O)c2nccc(OC)c2OCOC(C)=O)C(=O)O[C@@H](C)[C@@H]1Oc1ccccc1] | Example 11, Step 3A | White Foam |
| 24 | 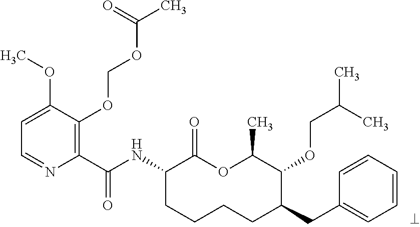 [SMILES: COc1ccnc(C(=O)N[C@H]2CCCC[C@H](Cc3ccccc3)[C@@H](OCC(C)C)[C@H](C)OC2=O)c1OCOC(C)=O] | Example 11, Step 3A | White Solid |
| 25 | 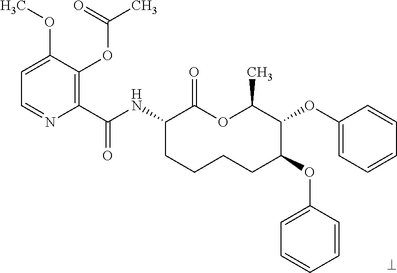 [SMILES: COc1ccnc(C(=O)N[C@H]2CCCC[C@H](Oc3ccccc3)[C@@H](Oc3ccccc3)[C@H](C)OC2=O)c1OC(C)=O] | Example 11, Step 3B | White Solid |
| 26 | 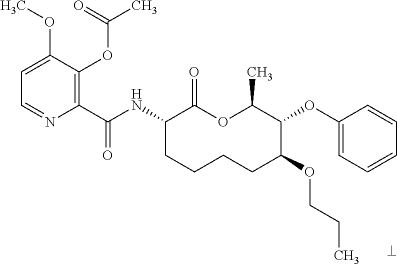 [SMILES: CCCO[C@H]1CCCC[C@H](NC(=O)c2nccc(OC)c2OC(C)=O)C(=O)O[C@@H](C)[C@@H]1Oc1ccccc1] | Example 11, Step 3B | White Solid |
| 27 | 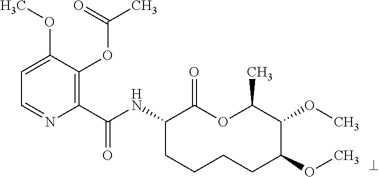 [SMILES: COc1ccnc(C(=O)N[C@H]2CCCC[C@H](OC)[C@@H](OC)[C@H](C)OC2=O)c1OC(C)=O] | Example 11, Step 3B | White Solid |
| 28 | 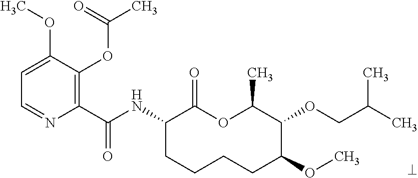 [SMILES: COc1ccnc(C(=O)N[C@H]2CCCC[C@H](OC)[C@@H](OCC(C)C)[C@H](C)OC2=O)c1OC(C)=O] | Example 11, Step 3B | White Solid |
| 29 | 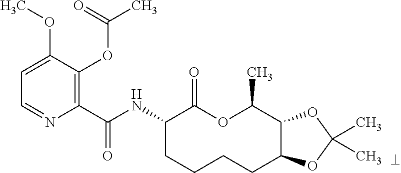 [SMILES: COc1ccnc(C(=O)N[C@H]2CCCC[C@@H]3OC(C)(C)O[C@H]3[C@H](C)OC2=O)c1OC(C)=O] | Example 11, Step 3B | White Solid |
| 30 | 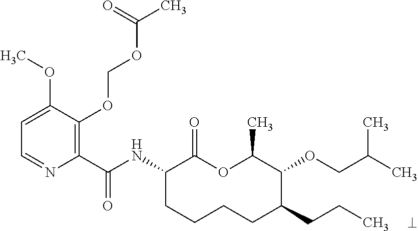 [SMILES: CCC[C@H]1CCCC[C@H](NC(=O)c2nccc(OC)c2OCOC(C)=O)C(=O)O[C@@H](C)[C@@H]1OCC(C)C] | Example 11, Step 3A | Light Yellow Solid |
| 31 | 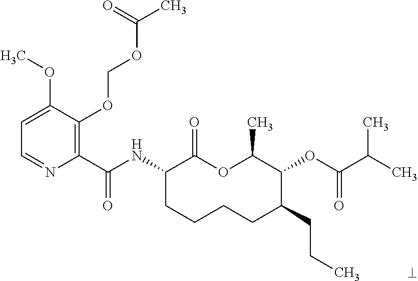 [SMILES: CCC[C@H]1CCCC[C@H](NC(=O)c2nccc(OC)c2OCOC(C)=O)C(=O)O[C@@H](C)[C@@H]1OC(=O)C(C)C] | Example 11, Step 3A | — |
| 32 | 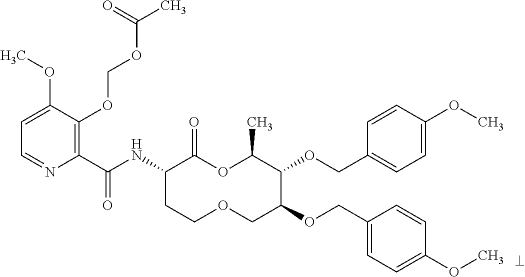 [SMILES: COc1ccc(CO[C@H]2[C@H](C)OC(=O)[C@@H](NC(=O)c3nccc(OC)c3OCOC(C)=O)CCOC[C@@H]2OCc2ccc(OC)cc2)cc1] | Example 11, Step 3A | White Solid |
| 33 | 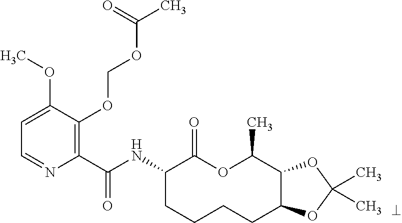 [SMILES: COc1ccnc(C(=O)N[C@H]2CCCC[C@@H]3OC(C)(C)O[C@H]3[C@H](C)OC2=O)c1OCOC(C)=O] | Example 11, Step 3A | Sticky Wax |
| 34 | 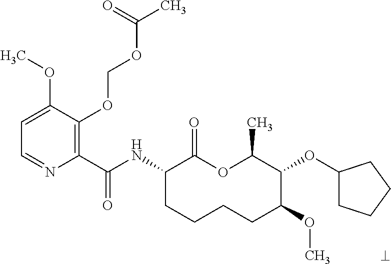 [SMILES: COc1ccnc(C(=O)N[C@H]2CCCC[C@H](OC)[C@@H](OC3CCCC3)[C@H](C)OC2=O)c1OCOC(C)=O] | Example 11, Step 3A | Colorless Semi- Solid |
| 35 | 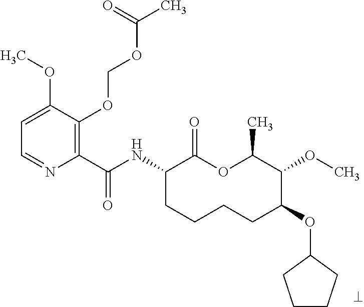 [SMILES: COc1ccnc(C(=O)N[C@H]2CCCC[C@H](OC3CCCC3)[C@@H](OC)[C@H](C)OC2=O)c1OCOC(C)=O] | Example 11, Step 3A | Pale Yellow Semi- Solid |
| 36 | 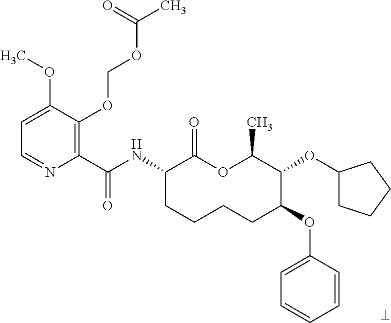 [SMILES: COc1ccnc(C(=O)N[C@H]2CCCC[C@H](Oc3ccccc3)[C@@H](OC3CCCC3)[C@H](C)OC2=O)c1OCOC(C)=O] | Example 11, Step 3A | Off White Solid |
| 37 | 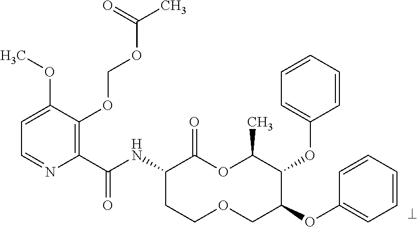 [SMILES: COc1ccnc(C(=O)N[C@H]2CCOC[C@H](Oc3ccccc3)[C@@H](Oc3ccccc3)[C@H](C)OC2=O)c1OCOC(C)=O] | Example 11, Step 3A | White Foam |
| 38 | 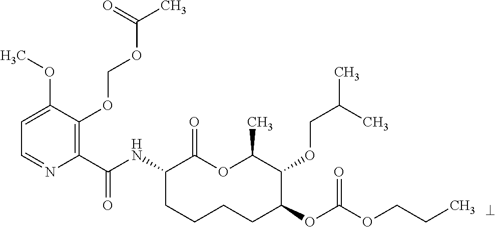 [SMILES: CCCOC(=O)O[C@H]1CCCC[C@H](NC(=O)c2nccc(OC)c2OCOC(C)=O)C(=O)O[C@@H](C)[C@@H]1OCC(C)C] | Example 11, Step 3A | White Solid |
| 39 | 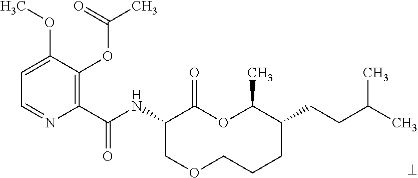 [SMILES: COc1ccnc(C(=O)N[C@H]2COCCC[C@@H](CCC(C)C)[C@H](C)OC2=O)c1OC(C)=O] | Example 11, Step 3B | White Powder |
| 40 | 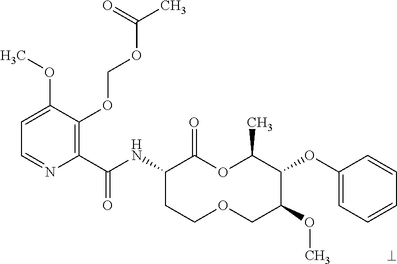 [SMILES: COc1ccnc(C(=O)N[C@H]2CCOC[C@H](OC)[C@@H](Oc3ccccc3)[C@H](C)OC2=O)c1OCOC(C)=O] | Example 11, Step 3A | White Solid |
| 41 | 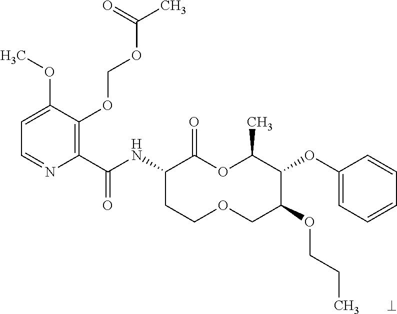 [SMILES: CCCO[C@H]1COCC[C@H](NC(=O)c2nccc(OC)c2OCOC(C)=O)C(=O)O[C@@H](C)[C@@H]1Oc1ccccc1] | Example 11, Step 3A | White Solid |
| 42 | 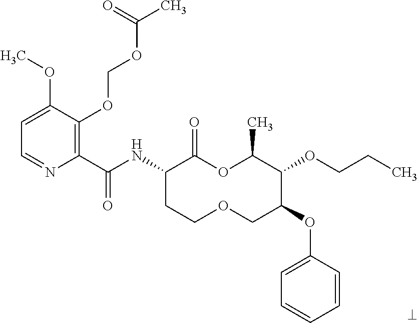 [SMILES: CCCO[C@H]1[C@H](C)OC(=O)[C@@H](NC(=O)c2nccc(OC)c2OCOC(C)=O)CCOC[C@@H]1Oc1ccccc1] | Example 11, Step 3A | White Solid |
| 43 | 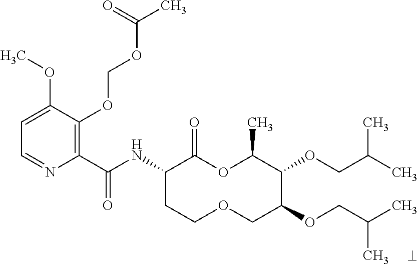 [SMILES: COc1ccnc(C(=O)N[C@H]2CCOC[C@H](OCC(C)C)[C@@H](OCC(C)C)[C@H](C)OC2=O)c1OCOC(C)=O] | Example 11, Step 3A | White Solid |
| 44 | 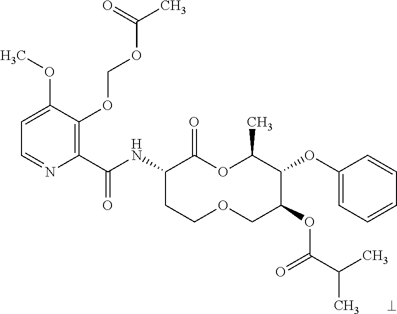 [SMILES: COc1ccnc(C(=O)N[C@H]2CCOC[C@H](OC(=O)C(C)C)[C@@H](Oc3ccccc3)[C@H](C)OC2=O)c1OCOC(C)=O] | Example 11, Step 3A | White Solid |
| 45 | 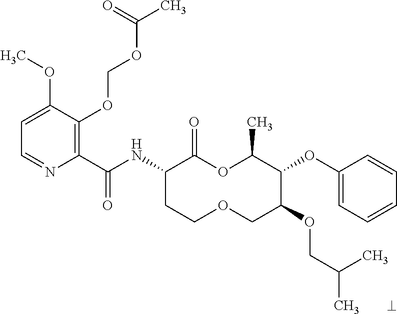 [SMILES: COc1ccnc(C(=O)N[C@H]2CCOC[C@H](OCC(C)C)[C@@H](Oc3ccccc3)[C@H](C)OC2=O)c1OCOC(C)=O] | Example 11, Step 3A | White Solid |
| 46 | 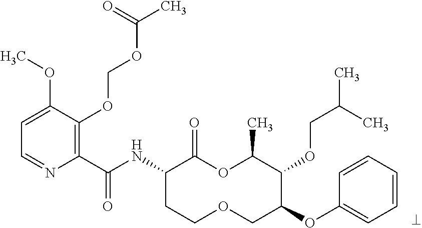 [SMILES: COc1ccnc(C(=O)N[C@H]2CCOC[C@H](Oc3ccccc3)[C@@H](OCC(C)C)[C@H](C)OC2=O)c1OCOC(C)=O] | Example 11, Step 3A | White Solid |
| 47 | 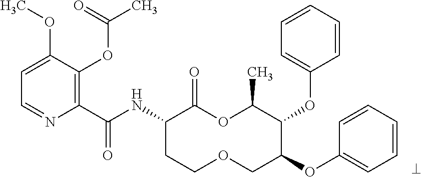 [SMILES: COc1ccnc(C(=O)N[C@H]2CCOC[C@H](Oc3ccccc3)[C@@H](Oc3ccccc3)[C@H](C)OC2=O)c1OC(C)=O] | Example 11, Step 3B | White Foam |
| 48 | 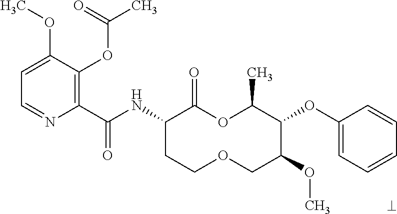 [SMILES: COc1ccnc(C(=O)N[C@H]2CCOC[C@H](OC)[C@@H](Oc3ccccc3)[C@H](C)OC2=O)c1OC(C)=O] | Example 11, Step 3B | White Foam |
| 49 | 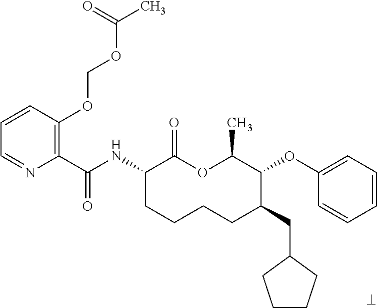 [SMILES: CC(=O)OCOc1cccnc1C(=O)N[C@H]1CCCC[C@H](CC2CCCC2)[C@@H](Oc2ccccc2)[C@H](C)OC1=O] | Example 11, Step 3A | White Solid |
| 50 | 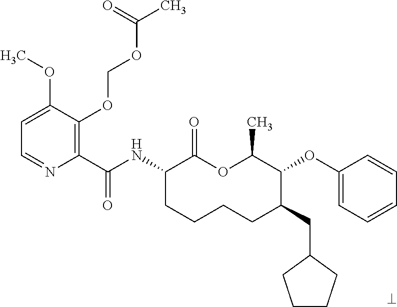 [SMILES: COc1ccnc(C(=O)N[C@H]2CCCC[C@H](CC3CCCC3)[C@@H](Oc3ccccc3)[C@H](C)OC2=O)c1OCOC(C)=O] | Example 11, Step 3A | White Solid |
| 51 | 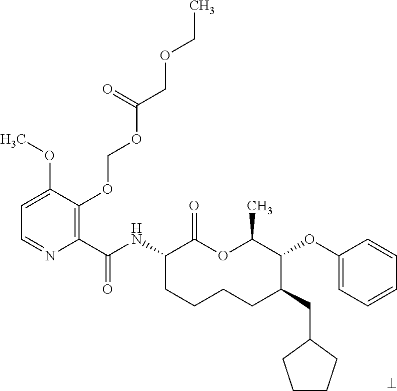 [SMILES: CCOCC(=O)OCOc1c(OC)ccnc1C(=O)N[C@H]1CCCC[C@H](CC2CCCC2)[C@@H](Oc2ccccc2)[C@H](C)OC1=O] | Example 11, Step 3C | White Solid |
| 52 | 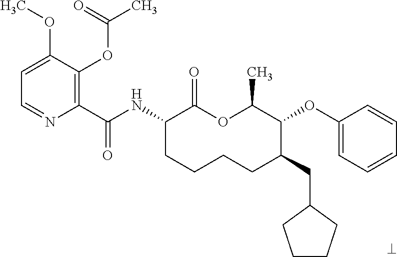 [SMILES: COc1ccnc(C(=O)N[C@H]2CCCC[C@H](CC3CCCC3)[C@@H](Oc3ccccc3)[C@H](C)OC2=O)c1OC(C)=O] | Example 11, Step 3B | White Solid |
| 53 | 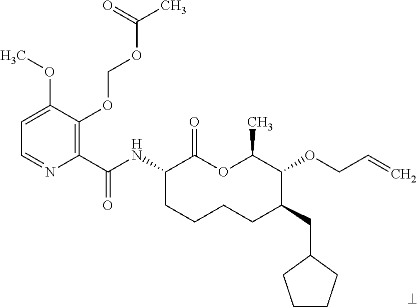 [SMILES: C=CCO[C@@H]1[C@@H](CC2CCCC2)CCCC[C@H](NC(=O)c2nccc(OC)c2OCOC(C)=O)C(=O)O[C@H]1C] | Example 11, Step 3A | White Solid |
| 54 | 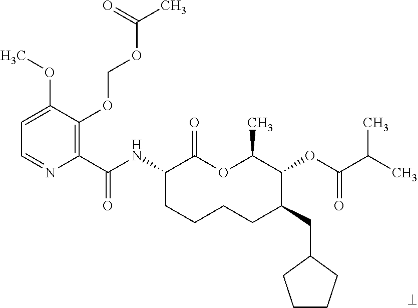 [SMILES: COc1ccnc(C(=O)N[C@H]2CCCC[C@H](CC3CCCC3)[C@@H](OC(=O)C(C)C)[C@H](C)OC2=O)c1OCOC(C)=O] | Example 11, Step 3A | White Solid |
| 55 | 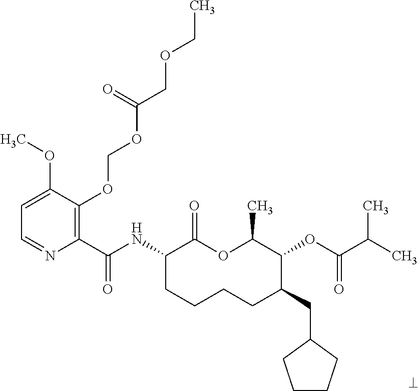 [SMILES: CCOCC(=O)OCOc1c(OC)ccnc1C(=O)N[C@H]1CCCC[C@H](CC2CCCC2)[C@@H](OC(=O)C(C)C)[C@H](C)OC1=O] | Example 11, Step 3C | — |
| 56 | 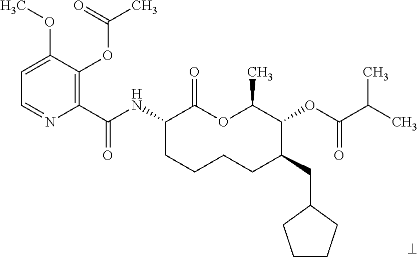 [SMILES: COc1ccnc(C(=O)N[C@H]2CCCC[C@H](CC3CCCC3)[C@@H](OC(=O)C(C)C)[C@H](C)OC2=O)c1OC(C)=O] | Example 11, Step 3B | White Solid |
| 57 | 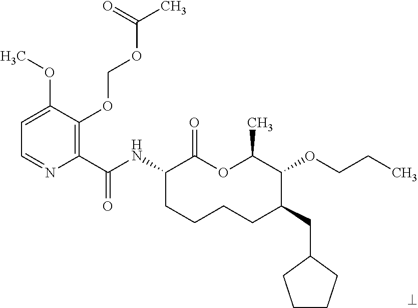 [SMILES: CCCO[C@@H]1[C@@H](CC2CCCC2)CCCC[C@H](NC(=O)c2nccc(OC)c2OCOC(C)=O)C(=O)O[C@H]1C] | Example 11, Step 3A | White Solid |
| 58 | 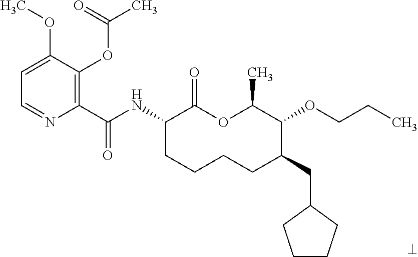 [SMILES: CCCO[C@@H]1[C@@H](CC2CCCC2)CCCC[C@H](NC(=O)c2nccc(OC)c2OC(C)=O)C(=O)O[C@H]1C] | Example 11, Step 3B | White Solid |
| 59 | 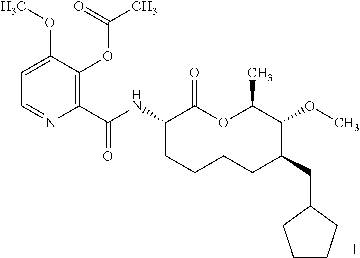 [SMILES: COc1ccnc(C(=O)N[C@H]2CCCC[C@H](CC3CCCC3)[C@@H](OC)[C@H](C)OC2=O)c1OC(C)=O] | Example 11, Step 3B | White Solid |
| 60 | 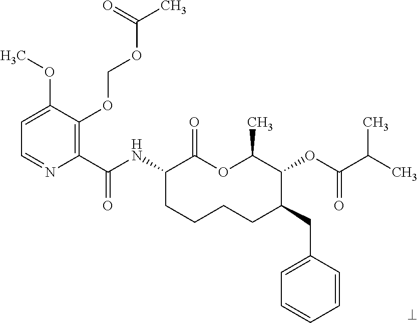 [SMILES: COc1ccnc(C(=O)N[C@H]2CCCC[C@H](Cc3ccccc3)[C@@H](OC(=O)C(C)C)[C@H](C)OC2=O)c1OCOC(C)=O] | Example 11, Step 3A | White Solid |
| 61 | 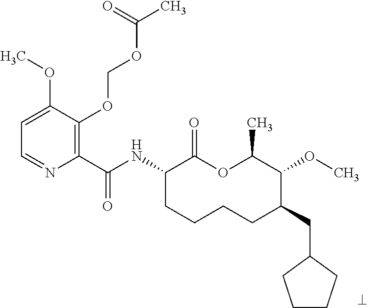 [SMILES: COc1ccnc(C(=O)N[C@H]2CCCC[C@H](CC3CCCC3)[C@@H](OC)[C@H](C)OC2=O)c1OCOC(C)=O] | Example 11, Step 3A | White Solid |
| 62 | 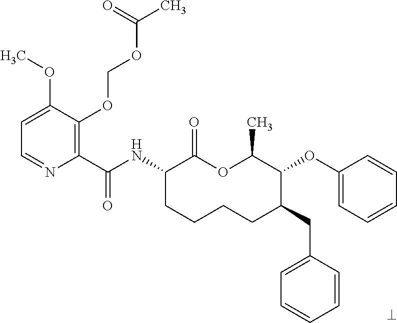 [SMILES: COc1ccnc(C(=O)N[C@H]2CCCC[C@H](Cc3ccccc3)[C@@H](Oc3ccccc3)[C@H](C)OC2=O)c1OCOC(C)=O] | Example 11, Step 3A | White Solid |
| 63 | 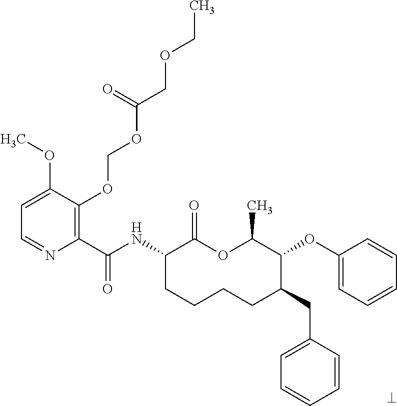 [SMILES: CCOCC(=O)OCOc1c(OC)ccnc1C(=O)N[C@H]1CCCC[C@H](Cc2ccccc2)[C@@H](Oc2ccccc2)[C@H](C)OC1=O] | Example 11, Step 3C | White Solid |
| 64 | 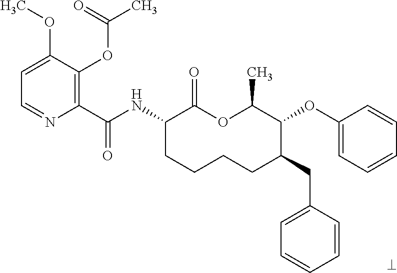 [SMILES: COc1ccnc(C(=O)N[C@H]2CCCC[C@H](Cc3ccccc3)[C@@H](Oc3ccccc3)[C@H](C)OC2=O)c1OC(C)=O] | Example 11, Step 3B | White Solid |
| 65 | 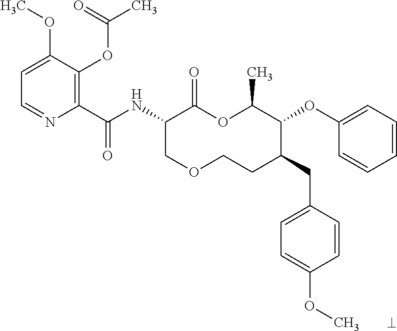 [SMILES: COc1ccc(C[C@H]2CCOC[C@H](NC(=O)c3nccc(OC)c3OC(C)=O)C(=O)O[C@@H](C)[C@@H]2Oc2ccccc2)cc1] | Example 11, Step 3B | White Solid |
| 66 | 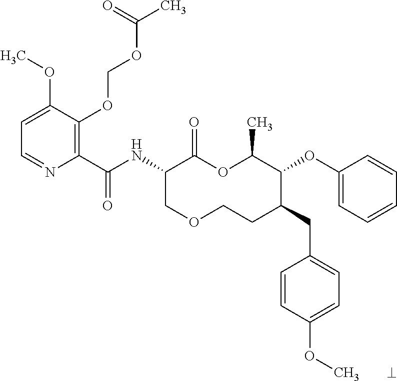 [SMILES: COc1ccc(C[C@H]2CCOC[C@H](NC(=O)c3nccc(OC)c3OCOC(C)=O)C(=O)O[C@@H](C)[C@@H]2Oc2ccccc2)cc1] | Example 11, Step 3A | White Solid |
| 67 | 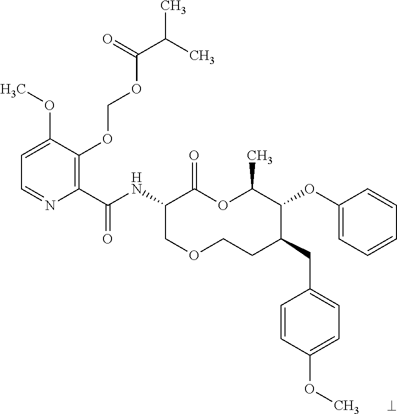 [SMILES: COc1ccc(C[C@H]2CCOC[C@H](NC(=O)c3nccc(OC)c3OCOC(=O)C(C)C)C(=O)O[C@@H](C)[C@@H]2Oc2ccccc2)cc1] | Example 11, Step 3D | White Solid |
| 68 | 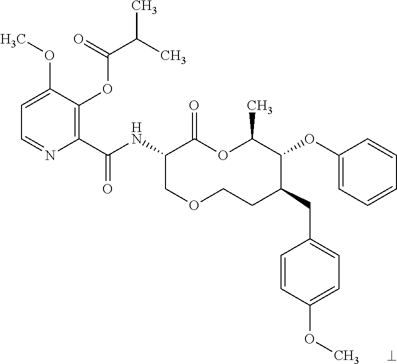 [SMILES: COc1ccc(C[C@H]2CCOC[C@H](NC(=O)c3nccc(OC)c3OC(=O)C(C)C)C(=O)O[C@@H](C)[C@@H]2Oc2ccccc2)cc1] | Example 11, Step 3B | White Solid |
| 69 | 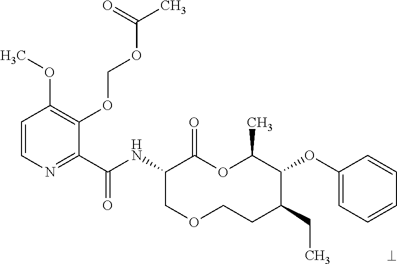 [SMILES: CC[C@H]1CCOC[C@H](NC(=O)c2nccc(OC)c2OCOC(C)=O)C(=O)O[C@@H](C)[C@@H]1Oc1ccccc1] | Example 11, Step 3A | Sticky Yellow Solid |
| 70 | 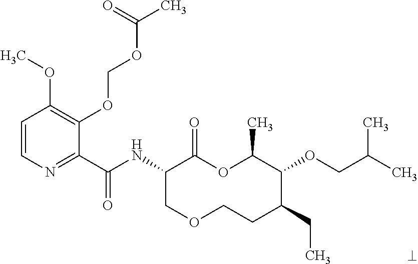 [SMILES: CC[C@H]1CCOC[C@H](NC(=O)c2nccc(OC)c2OCOC(C)=O)C(=O)O[C@@H](C)[C@@H]1OCC(C)C] | Example 11, Step 3A | Colorless Semi Solid |
| 71 | 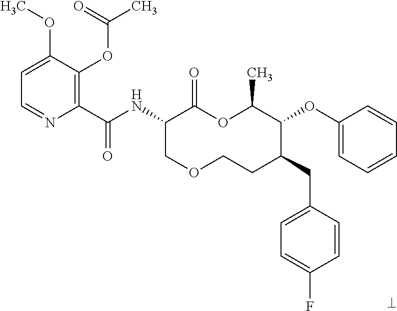 [SMILES: COc1ccnc(C(=O)N[C@H]2COCC[C@H](Cc3ccc(F)cc3)[C@@H](Oc3ccccc3)[C@H](C)OC2=O)c1OC(C)=O] | Example 11, Step 3B | White Solid |
| 72 | 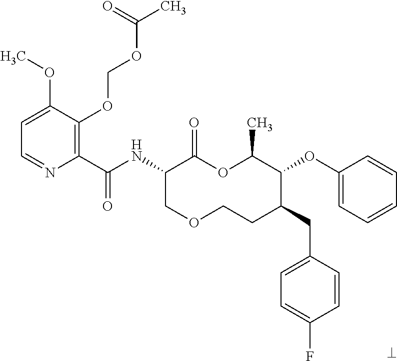 [SMILES: COc1ccnc(C(=O)N[C@H]2COCC[C@H](Cc3ccc(F)cc3)[C@@H](Oc3ccccc3)[C@H](C)OC2=O)c1OCOC(C)=O] | Example 11, Step 3A | White Solid |
| 73 | 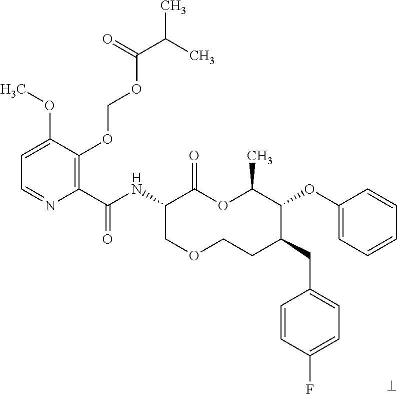 [SMILES: COc1ccnc(C(=O)N[C@H]2COCC[C@H](Cc3ccc(F)cc3)[C@@H](Oc3ccccc3)[C@H](C)OC2=O)c1OCOC(=O)C(C)C] | Example 11, Step 3D | White Solid |
| 74 | 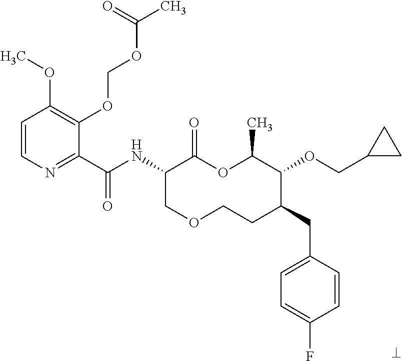 [SMILES: COc1ccnc(C(=O)N[C@H]2COCC[C@H](Cc3ccc(F)cc3)[C@@H](OCC3CC3)[C@H](C)OC2=O)c1OCOC(C)=O] | Example 11, Step 3A | Colorless Semi Solid |
| 75 | 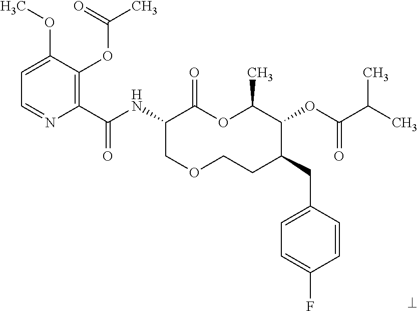 [SMILES: COc1ccnc(C(=O)N[C@H]2COCC[C@H](Cc3ccc(F)cc3)[C@@H](OC(=O)C(C)C)[C@H](C)OC2=O)c1OC(C)=O] | Example 11, Step 3B | White Solid |
| 76 | 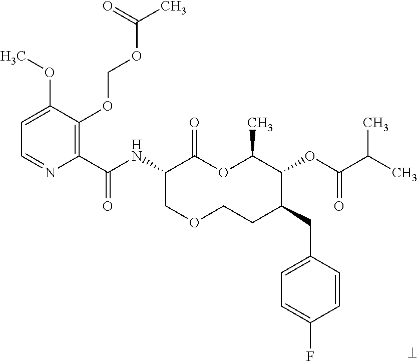 [SMILES: COc1ccnc(C(=O)N[C@H]2COCC[C@H](Cc3ccc(F)cc3)[C@@H](OC(=O)C(C)C)[C@H](C)OC2=O)c1OCOC(C)=O] | Example 11, Step 3A | White Solid |
| 77 | 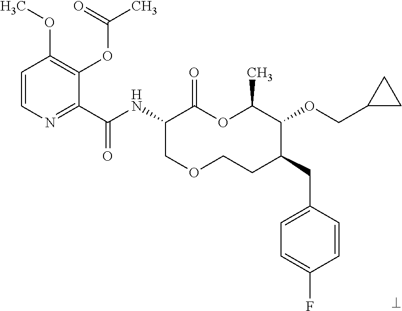 [SMILES: COc1ccnc(C(=O)N[C@H]2COCC[C@H](Cc3ccc(F)cc3)[C@@H](OCC3CC3)[C@H](C)OC2=O)c1OC(C)=O] | Example 11, Step 3B | White Solid |
| 78 | 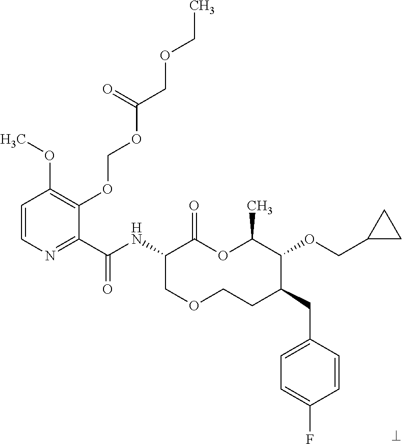 [SMILES: CCOCC(=O)OCOc1c(OC)ccnc1C(=O)N[C@H]1COCC[C@H](Cc2ccc(F)cc2)[C@@H](OCC2CC2)[C@H](C)OC1=O] | Example 11, Step 3C | Colorless Semi Solid |
| 79 | 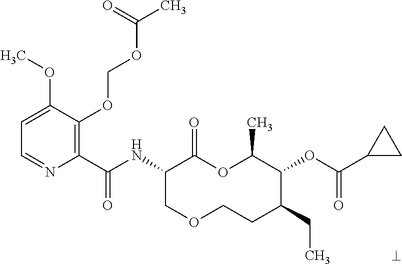 [SMILES: CC[C@H]1CCOC[C@H](NC(=O)c2nccc(OC)c2OCOC(C)=O)C(=O)O[C@@H](C)[C@@H]1OC(=O)C1CC1] | Example 11, Step 3A | Colorless Semi Solid |
| 80 | 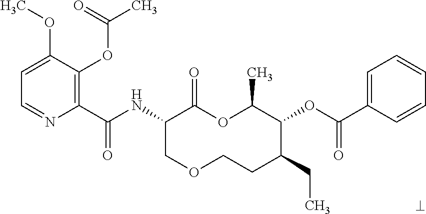 [SMILES: CC[C@H]1CCOC[C@H](NC(=O)c2nccc(OC)c2OC(C)=O)C(=O)O[C@@H](C)[C@@H]1OC(=O)c1ccccc1] | Example 11, Step 3B | Light Yellow Oil |
| 81 | 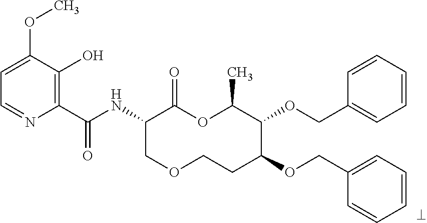 [SMILES: COc1ccnc(C(=O)N[C@H]2COCC[C@H](OCc3ccccc3)[C@@H](OCc3ccccc3)[C@H](C)OC2=O)c1O] | Example 11, Step 2 | Amorphous White Solid |
| 82 | 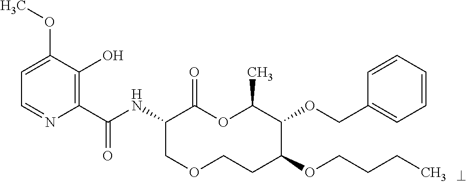 [SMILES: CCCCO[C@H]1CCOC[C@H](NC(=O)c2nccc(OC)c2O)C(=O)O[C@@H](C)[C@@H]1OCc1ccccc1] | Example 11, Step 2 | Sticky White Solid |
| 83 | 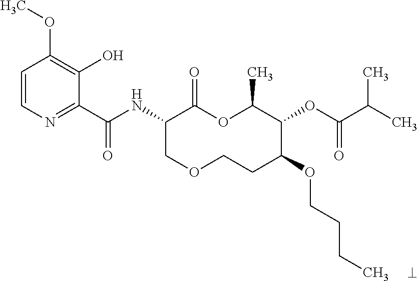 [SMILES: CCCCO[C@H]1CCOC[C@H](NC(=O)c2nccc(OC)c2O)C(=O)O[C@@H](C)[C@@H]1OC(=O)C(C)C] | Example 11, Step 2 | Sticky White Solid |
| 84 | 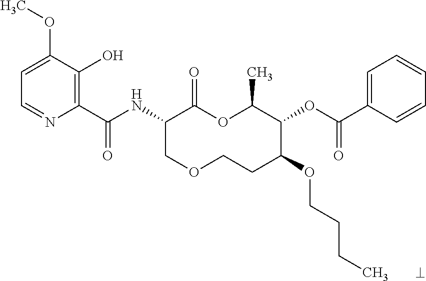 [SMILES: CCCCO[C@H]1CCOC[C@H](NC(=O)c2nccc(OC)c2O)C(=O)O[C@@H](C)[C@@H]1OC(=O)c1ccccc1] | Example 11, Step 2 | Sticky White Solid |
| 85 | 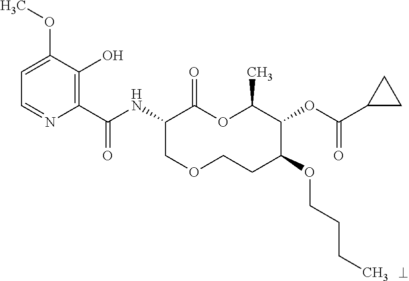 [SMILES: CCCCO[C@H]1CCOC[C@H](NC(=O)c2nccc(OC)c2O)C(=O)O[C@@H](C)[C@@H]1OC(=O)C1CC1] | Example 11, Step 2 | Fluffy White Solid |
| 86 | 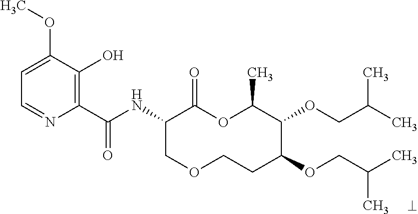 [SMILES: COc1ccnc(C(=O)N[C@H]2COCC[C@H](OCC(C)C)[C@@H](OCC(C)C)[C@H](C)OC2=O)c1O] | Example 11, Step 2 | Fluffy White Solid |
| 87 | 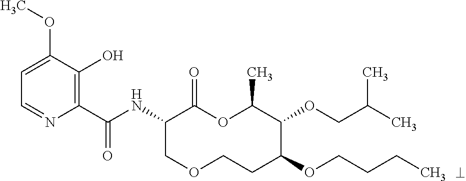 [SMILES: CCCCO[C@H]1CCOC[C@H](NC(=O)c2nccc(OC)c2O)C(=O)O[C@@H](C)[C@@H]1OCC(C)C] | Example 11, Step 2 | Sticky White Foam |
| 88 | 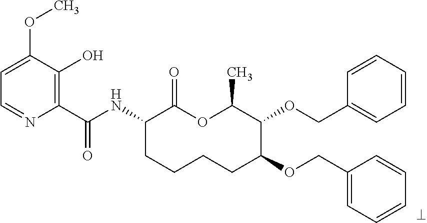 [SMILES: COc1ccnc(C(=O)N[C@H]2CCCC[C@H](OCc3ccccc3)[C@@H](OCc3ccccc3)[C@H](C)OC2=O)c1O] | Example 11, Step 2 | Sticky White Solid |
| 89 | 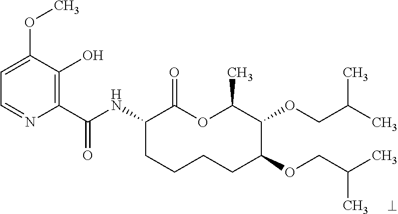 [SMILES: COc1ccnc(C(=O)N[C@H]2CCCC[C@H](OCC(C)C)[C@@H](OCC(C)C)[C@H](C)OC2=O)c1O] | Example 11, Step 2 | White Solid |
| 90 | 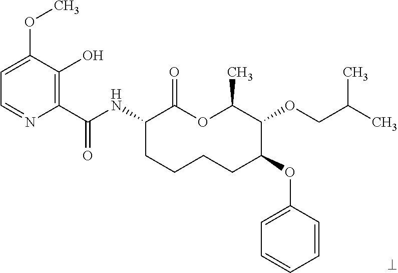 [SMILES: COc1ccnc(C(=O)N[C@H]2CCCC[C@H](Oc3ccccc3)[C@@H](OCC(C)C)[C@H](C)OC2=O)c1O] | Example 11, Step 2 | Sticky Clear Oil |
| 91 | 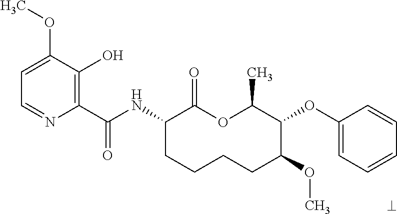 [SMILES: COc1ccnc(C(=O)N[C@H]2CCCC[C@H](OC)[C@@H](Oc3ccccc3)[C@H](C)OC2=O)c1O] | Example 11, Step 2 | Clear Colorless Oil |
| 92 | 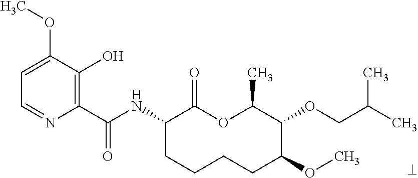 [SMILES: COc1ccnc(C(=O)N[C@H]2CCCC[C@H](OC)[C@@H](OCC(C)C)[C@H](C)OC2=O)c1O] | Example 11, Step 2 | Pale Yellow Solid |
| 93 | 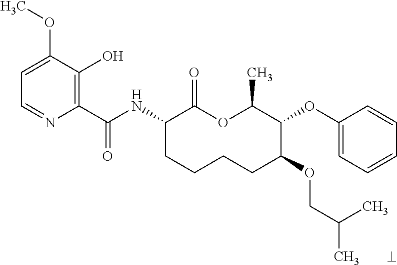 [SMILES: COc1ccnc(C(=O)N[C@H]2CCCC[C@H](OCC(C)C)[C@@H](Oc3ccccc3)[C@H](C)OC2=O)c1O] | Example 11, Step 2 | Sticky White Oil |
| 94 | 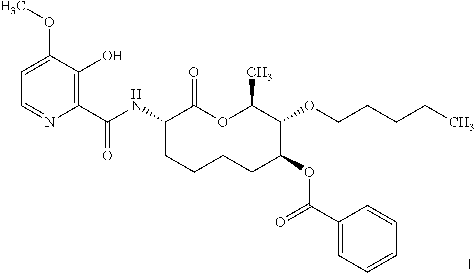 [SMILES: CCCCCO[C@H]1[C@H](C)OC(=O)[C@@H](NC(=O)c2nccc(OC)c2O)CCCC[C@@H]1OC(=O)c1ccccc1] | Example 11, Step 2 | Sticky Clear Oil |
| 95 | 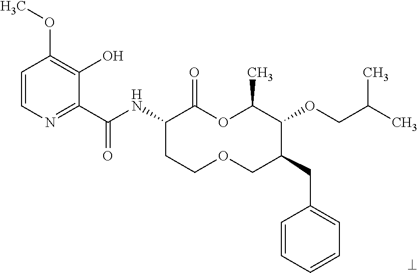 [SMILES: COc1ccnc(C(=O)N[C@H]2CCOC[C@H](Cc3ccccc3)[C@@H](OCC(C)C)[C@H](C)OC2=O)c1O] | Example 11, Step 2 | White Solid |
| 96 | 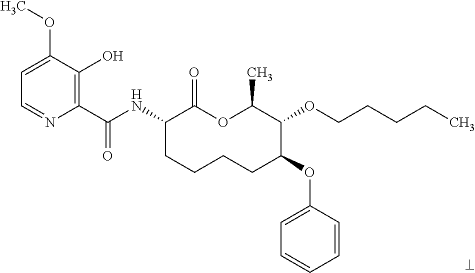 [SMILES: CCCCCO[C@H]1[C@H](C)OC(=O)[C@@H](NC(=O)c2nccc(OC)c2O)CCCC[C@@H]1Oc1ccccc1] | Example 11, Step 2 | Clear Colorless Oil |
| 97 | 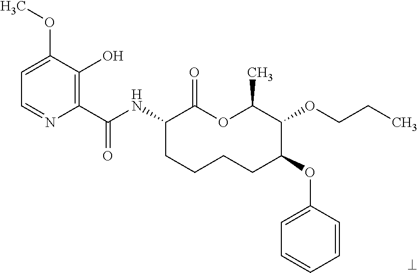 [SMILES: CCCO[C@H]1[C@H](C)OC(=O)[C@@H](NC(=O)c2nccc(OC)c2O)CCCC[C@@H]1Oc1ccccc1] | Example 11, Step 2 | Clear Colorless Oil |
| 98 | 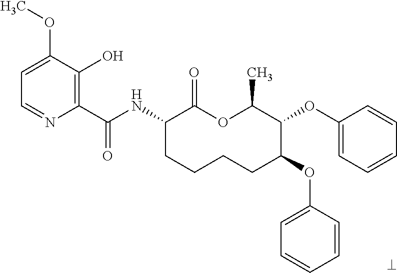 [SMILES: COc1ccnc(C(=O)N[C@H]2CCCC[C@H](Oc3ccccc3)[C@@H](Oc3ccccc3)[C@H](C)OC2=O)c1O] | Example 11, Step 2 | White Foam |
| 99 | 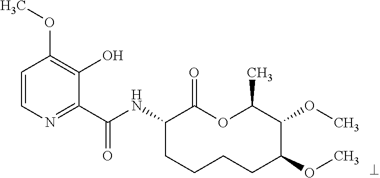 [SMILES: COc1ccnc(C(=O)N[C@H]2CCCC[C@H](OC)[C@@H](OC)[C@H](C)OC2=O)c1O] | Example 11, Step 2 | White Foam |
| 100 | 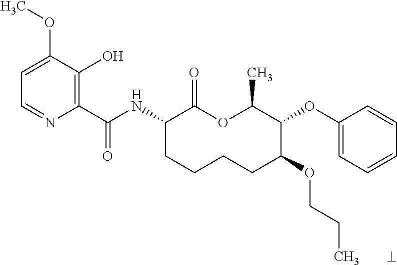 [SMILES: CCCO[C@H]1CCCC[C@H](NC(=O)c2nccc(OC)c2O)C(=O)O[C@@H](C)[C@@H]1Oc1ccccc1] | Example 11, Step 2 | White Foamy Solid |
| 101 | 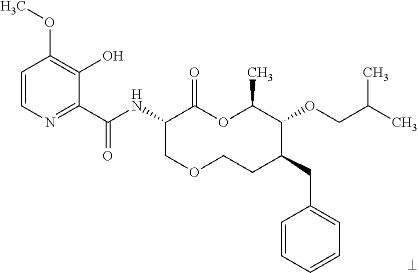 [SMILES: COc1ccnc(C(=O)N[C@H]2COCC[C@H](Cc3ccccc3)[C@@H](OCC(C)C)[C@H](C)OC2=O)c1O] | Example 11, Step 2 | White Solid |
| 102 | 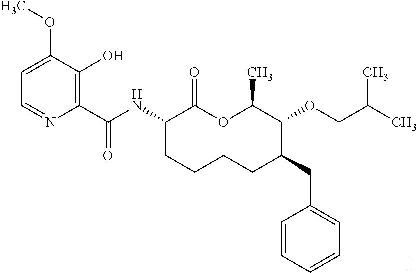 [SMILES: COc1ccnc(C(=O)N[C@H]2CCCC[C@H](Cc3ccccc3)[C@@H](OCC(C)C)[C@H](C)OC2=O)c1O] | Example 11, Step 2 | Colorless Oil |
| 103 | 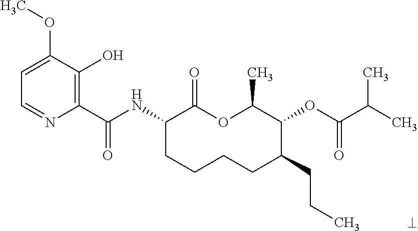 [SMILES: CCC[C@H]1CCCC[C@H](NC(=O)c2nccc(OC)c2O)C(=O)O[C@@H](C)[C@@H]1OC(=O)C(C)C] | Example 11, Step 2 | White Solid |
| 104 | 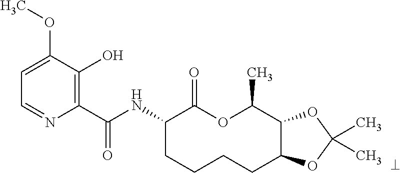 [SMILES: COc1ccnc(C(=O)N[C@H]2CCCC[C@@H]3OC(C)(C)O[C@H]3[C@H](C)OC2=O)c1O] | Example 11, Step 2 | White Solid |
| 105 | 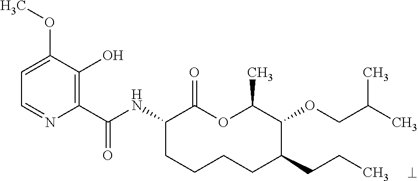 [SMILES: CCC[C@H]1CCCC[C@H](NC(=O)c2nccc(OC)c2O)C(=O)O[C@@H](C)[C@@H]1OCC(C)C] | Example 11, Step 2 | White Solid |
| 106 | 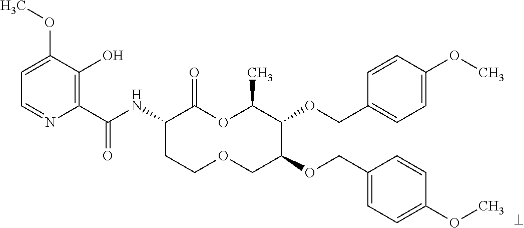 [SMILES: COc1ccc(CO[C@H]2[C@H](C)OC(=O)[C@@H](NC(=O)c3nccc(OC)c3O)CCOC[C@@H]2OCc2ccc(OC)cc2)cc1] | Example 11, Step 2 | Off White Solid |
| 107 | 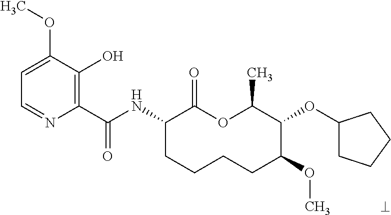 [SMILES: COc1ccnc(C(=O)N[C@H]2CCCC[C@H](OC)[C@@H](OC3CCCC3)[C@H](C)OC2=O)c1O] | Example 11, Step 2 | Off White Sticky Solid |
| 108 | 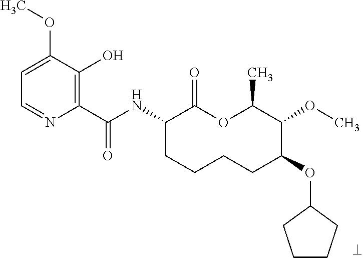 [SMILES: COc1ccnc(C(=O)N[C@H]2CCCC[C@H](OC3CCCC3)[C@@H](OC)[C@H](C)OC2=O)c1O] | Example 11, Step 2 | Off White Solid |
| 109 | 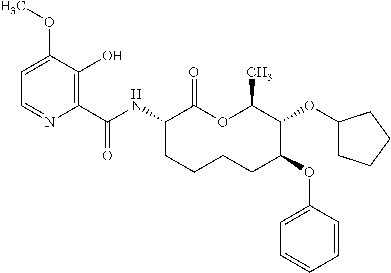 [SMILES: COc1ccnc(C(=O)N[C@H]2CCCC[C@H](Oc3ccccc3)[C@@H](OC3CCCC3)[C@H](C)OC2=O)c1O] | Example 11, Step 2 | White Foam |
| 110 | 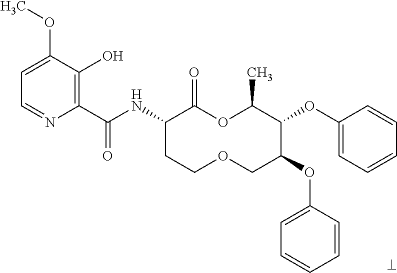 [SMILES: COc1ccnc(C(=O)N[C@H]2CCOC[C@H](Oc3ccccc3)[C@@H](Oc3ccccc3)[C@H](C)OC2=O)c1O] | Example 11, Step 2 | White Solid |
| 111 | 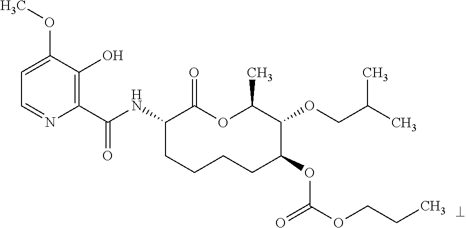 [SMILES: CCCOC(=O)O[C@H]1CCCC[C@H](NC(=O)c2nccc(OC)c2O)C(=O)O[C@@H](C)[C@@H]1OCC(C)C] | Example 11, Step 2 | White Foam |
| 112 | 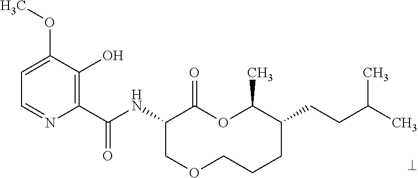 [SMILES: COc1ccnc(C(=O)N[C@H]2COCCC[C@@H](CCC(C)C)[C@H](C)OC2=O)c1O] | Example 11, Step 2 | Sticky White Solid |
| 113 | 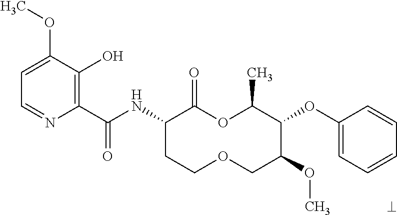 [SMILES: COc1ccnc(C(=O)N[C@H]2CCOC[C@H](OC)[C@@H](Oc3ccccc3)[C@H](C)OC2=O)c1O] | Example 11, Step 2 | Off White Glass |
| 114 | 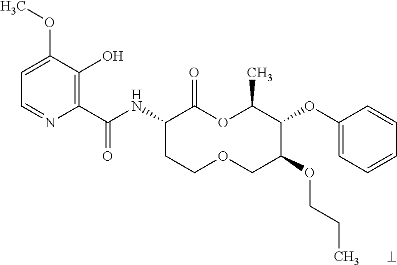 [SMILES: CCCO[C@H]1COCC[C@H](NC(=O)c2nccc(OC)c2O)C(=O)O[C@@H](C)[C@@H]1Oc1ccccc1] | Example 11, Step 2 | White Solid |
| 115 | 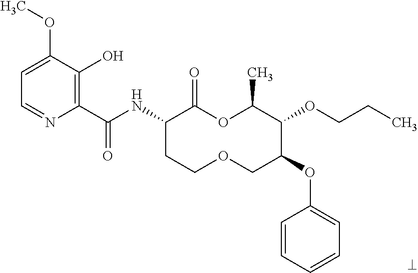 [SMILES: CCCO[C@H]1[C@H](C)OC(=O)[C@@H](NC(=O)c2nccc(OC)c2O)CCOC[C@@H]1Oc1ccccc1] | Example 11, Step 2 | White Solid |
| 116 | 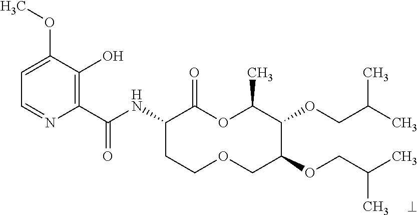 [SMILES: COc1ccnc(C(=O)N[C@H]2CCOC[C@H](OCC(C)C)[C@@H](OCC(C)C)[C@H](C)OC2=O)c1O] | Example 11, Step 2 | Yellow/ Off-White Solid |
| 117 | 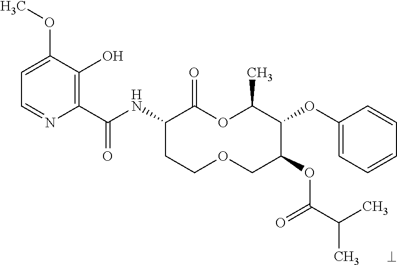 [SMILES: COc1ccnc(C(=O)N[C@H]2CCOC[C@H](OC(=O)C(C)C)[C@@H](Oc3ccccc3)[C@H](C)OC2=O)c1O] | Example 11, Step 2 | Off White Solid |
| 118 | 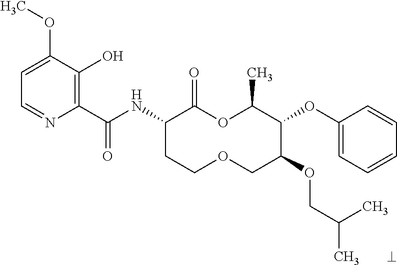 [SMILES: COc1ccnc(C(=O)N[C@H]2CCOC[C@H](OCC(C)C)[C@@H](Oc3ccccc3)[C@H](C)OC2=O)c1O] | Example 11, Step 2 | White Solid |
| 119 | 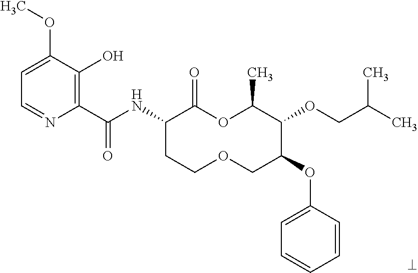 [SMILES: COc1ccnc(C(=O)N[C@H]2CCOC[C@H](Oc3ccccc3)[C@@H](OCC(C)C)[C@H](C)OC2=O)c1O] | Example 11, Step 2 | White Solid |
| 120 | 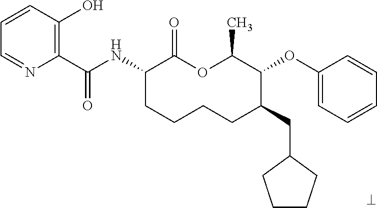 [SMILES: C[C@@H]1OC(=O)[C@@H](NC(=O)c2ncccc2O)CCCC[C@H](CC2CCCC2)[C@H]1Oc1ccccc1] | Example 11, Step 2 | White Solid |
| 121 | 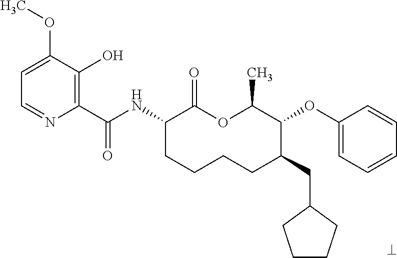 [SMILES: COc1ccnc(C(=O)N[C@H]2CCCC[C@H](CC3CCCC3)[C@@H](Oc3ccccc3)[C@H](C)OC2=O)c1O] | Example 11, Step 2 | White Solid |
| 122 | 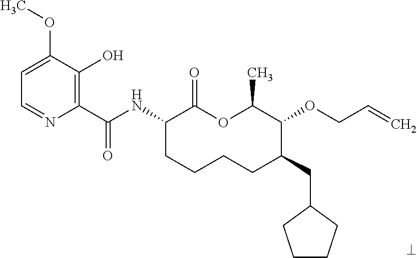 [SMILES: C=CCO[C@@H]1[C@@H](CC2CCCC2)CCCC[C@H](NC(=O)c2nccc(OC)c2O)C(=O)O[C@H]1C] | Example 11, Step 2 | White Solid |
| 123 | 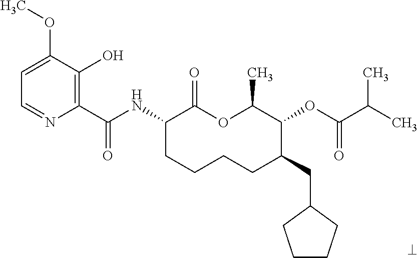 [SMILES: COc1ccnc(C(=O)N[C@H]2CCCC[C@H](CC3CCCC3)[C@@H](OC(=O)C(C)C)[C@H](C)OC2=O)c1O] | Example 11, Step 2 | Colorless Oil |
| 124 | 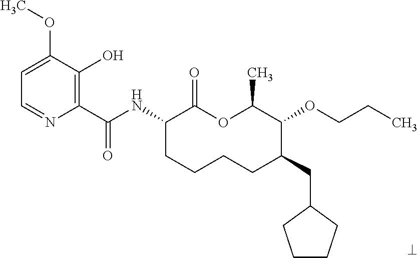 [SMILES: CCCO[C@@H]1[C@@H](CC2CCCC2)CCCC[C@H](NC(=O)c2nccc(OC)c2O)C(=O)O[C@H]1C] | Example 11, Step 2 | White Solid |
| 125 | 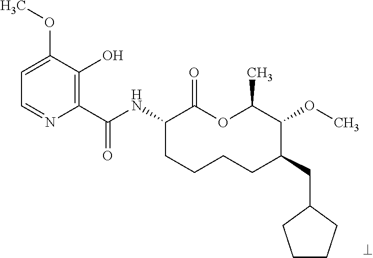 [SMILES: COc1ccnc(C(=O)N[C@H]2CCCC[C@H](CC3CCCC3)[C@@H](OC)[C@H](C)OC2=O)c1O] | Example 11, Step 2 | White Solid |
| 126 | 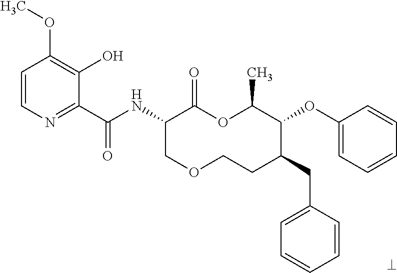 [SMILES: COc1ccnc(C(=O)N[C@H]2COCC[C@H](Cc3ccccc3)[C@@H](Oc3ccccc3)[C@H](C)OC2=O)c1O] | Example 11, Step 2 | White Solid |
| 127 | 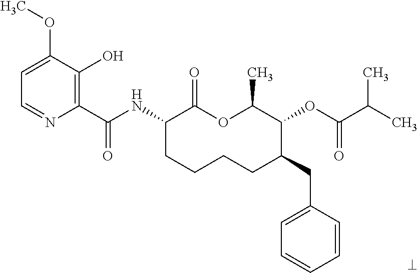 [SMILES: COc1ccnc(C(=O)N[C@H]2CCCC[C@H](Cc3ccccc3)[C@@H](OC(=O)C(C)C)[C@H](C)OC2=O)c1O] | Example 11, Step 2 | White Solid |
| 128 | 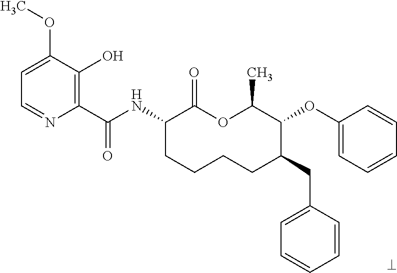 [SMILES: COc1ccnc(C(=O)N[C@H]2CCCC[C@H](Cc3ccccc3)[C@@H](Oc3ccccc3)[C@H](C)OC2=O)c1O] | Example 11, Step 2 | White Solid |
| 129 | 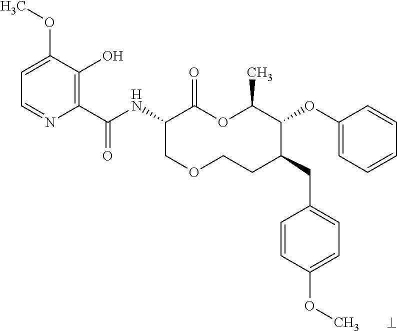 [SMILES: COc1ccc(C[C@H]2CCOC[C@H](NC(=O)c3nccc(OC)c3O)C(=O)O[C@@H](C)[C@@H]2Oc2ccccc2)cc1] | Example 11, Step 2 | White Solid |
| 130 | 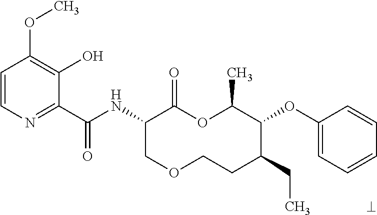 [SMILES: CC[C@H]1CCOC[C@H](NC(=O)c2nccc(OC)c2O)C(=O)O[C@@H](C)[C@@H]1Oc1ccccc1] | Example 11, Step 2 | Colorless Semi Solid |
| 131 | 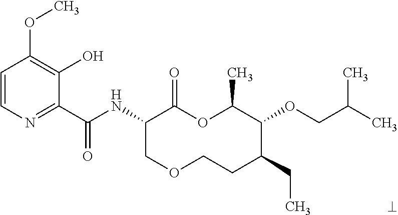 [SMILES: CC[C@H]1CCOC[C@H](NC(=O)c2nccc(OC)c2O)C(=O)O[C@@H](C)[C@@H]1OCC(C)C] | Example 11, Step 2 | Colorless Semi Solid |
| 132 | 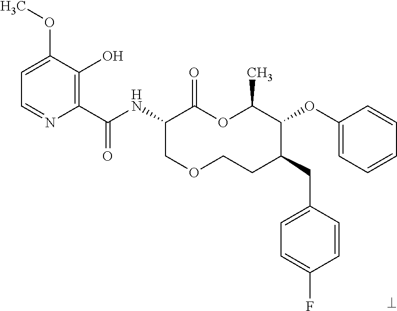 [SMILES: COc1ccnc(C(=O)N[C@H]2COCC[C@H](Cc3ccc(F)cc3)[C@@H](Oc3ccccc3)[C@H](C)OC2=O)c1O] | Example 11, Step 2 | White Solid |
| 133 | 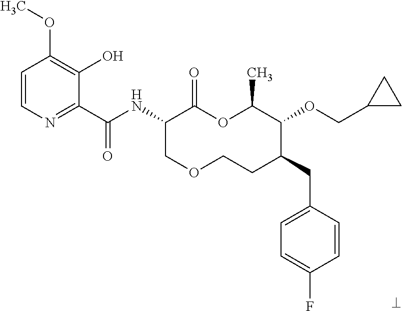 [SMILES: COc1ccnc(C(=O)N[C@H]2COCC[C@H](Cc3ccc(F)cc3)[C@@H](OCC3CC3)[C@H](C)OC2=O)c1O] | Example 11, Step 2 | White Solid |
| 134 | 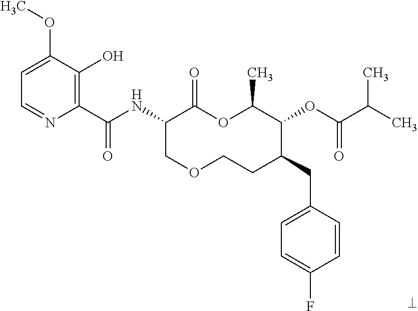 [SMILES: COc1ccnc(C(=O)N[C@H]2COCC[C@H](Cc3ccc(F)cc3)[C@@H](OC(=O)C(C)C)[C@H](C)OC2=O)c1O] | Example 11, Step 2 | White Solid |
| 135 | 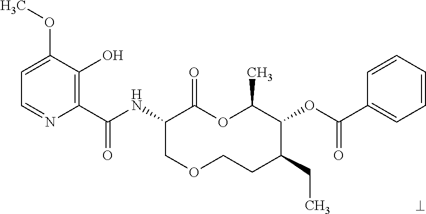 [SMILES: CC[C@H]1CCOC[C@H](NC(=O)c2nccc(OC)c2O)C(=O)O[C@@H](C)[C@@H]1OC(=O)c1ccccc1] | Example 11, Step 2 | White Solid |
| 136 | 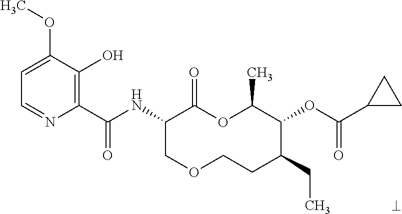 [SMILES: CC[C@H]1CCOC[C@H](NC(=O)c2nccc(OC)c2O)C(=O)O[C@@H](C)[C@@H]1OC(=O)C1CC1] | Example 11, Step 2 | White Solid |
| 137 | 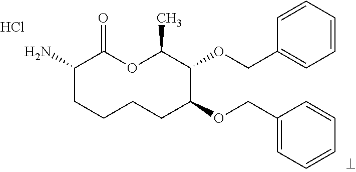 [SMILES: Cl ; C[C@@H]1OC(=O)[C@@H](N)CCCC[C@H](OCc2ccccc2)[C@H]1OCc1ccccc1] | Example 11, Step 1 | Light Yellow Solid |
| 138 | 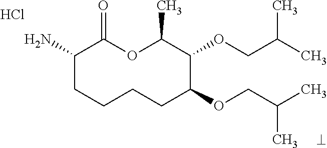 [SMILES: Cl ; CC(C)CO[C@H]1[C@H](C)OC(=O)[C@@H](N)CCCC[C@@H]1OCC(C)C] | Example 11, Step 1 | Chalky Yellow Solid |
| 139 | 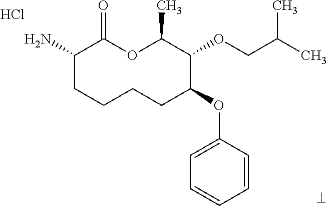 [SMILES: Cl ; CC(C)CO[C@H]1[C@H](C)OC(=O)[C@@H](N)CCCC[C@@H]1Oc1ccccc1] | Example 11, Step 1 | Chalky Off White Solid |
| 140 | 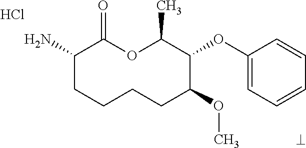 [SMILES: Cl ; CO[C@H]1CCCC[C@H](N)C(=O)O[C@@H](C)[C@@H]1Oc1ccccc1] | Example 11, Step 1 | Chalky Yellow Solid |
| 141 | 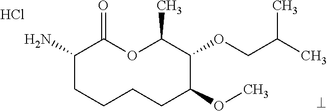 [SMILES: Cl ; CO[C@H]1CCCC[C@H](N)C(=O)O[C@@H](C)[C@@H]1OCC(C)C] | Example 11, Step 1 | White Solid |
| 142 | 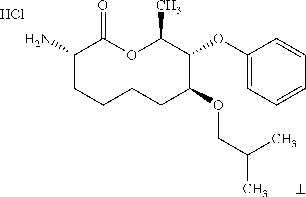 [SMILES: Cl ; CC(C)CO[C@H]1CCCC[C@H](N)C(=O)O[C@@H](C)[C@@H]1Oc1ccccc1] | Example 11, Step 1 | Chalky Off White Solid |
| 143 | 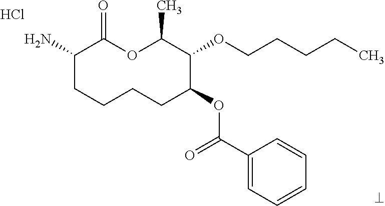 [SMILES: Cl ; CCCCCO[C@H]1[C@H](C)OC(=O)[C@@H](N)CCCC[C@@H]1OC(=O)c1ccccc1] | Example 11, Step 1 | Chalky White Solid |
| 144 | 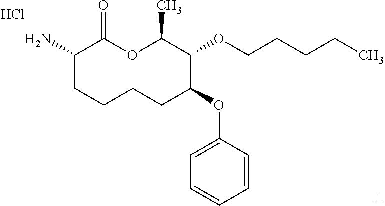 [SMILES: Cl ; CCCCCO[C@H]1[C@H](C)OC(=O)[C@@H](N)CCCC[C@@H]1Oc1ccccc1] | Example 11, Step 1 | Chalky White Solid |
| 145 | 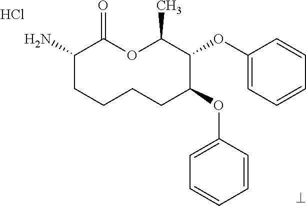 [SMILES: Cl ; C[C@@H]1OC(=O)[C@@H](N)CCCC[C@H](Oc2ccccc2)[C@H]1Oc1ccccc1] | Example 11, Step 1 | — |
| 146 | 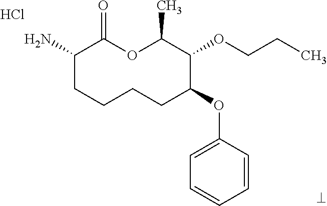 [SMILES: Cl ; CCCO[C@H]1[C@H](C)OC(=O)[C@@H](N)CCCC[C@@H]1Oc1ccccc1] | Example 11, Step 1 | Chalky Yellow Solid |
| 147 | 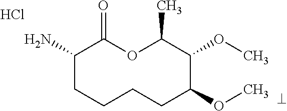 [SMILES: Cl ; CO[C@H]1[C@H](C)OC(=O)[C@@H](N)CCCC[C@@H]1OC] | Example 11, Step 1 | White Powder |
| 148 | 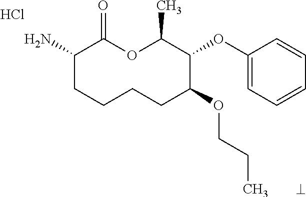 [SMILES: Cl ; CCCO[C@H]1CCCC[C@H](N)C(=O)O[C@@H](C)[C@@H]1Oc1ccccc1] | Example 11, Step 1 | White Solid |
| 149 | 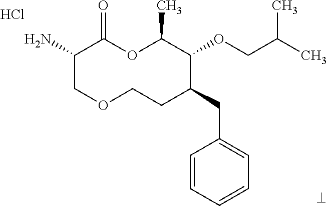 [SMILES: Cl ; CC(C)CO[C@@H]1[C@@H](Cc2ccccc2)CCOC[C@H](N)C(=O)O[C@H]1C] | Example 11, Step 1 | White Solid |
| 150 | 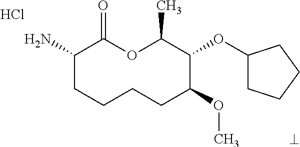 [SMILES: Cl ; CO[C@H]1CCCC[C@H](N)C(=O)O[C@@H](C)[C@@H]1OC1CCCC1] | Example 11, Step 1 | White Solid |
| 151 | 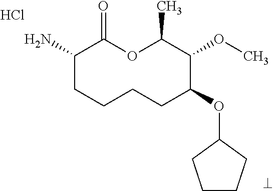 [SMILES: Cl ; CO[C@H]1[C@H](C)OC(=O)[C@@H](N)CCCC[C@@H]1OC1CCCC1] | Example 11, Step 1 | White Solid |
| 152 | 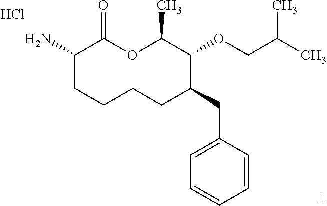 [SMILES: Cl ; CC(C)CO[C@@H]1[C@@H](Cc2ccccc2)CCCC[C@H](N)C(=O)O[C@H]1C] | Example 11, Step 1 | White Solid |
| 153 | 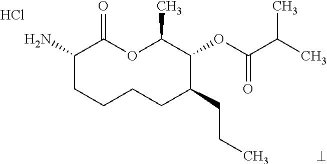 [SMILES: Cl ; CCC[C@H]1CCCC[C@H](N)C(=O)O[C@@H](C)[C@@H]1OC(=O)C(C)C] | Example 11, Step 1 | White Solid |
| 154 | 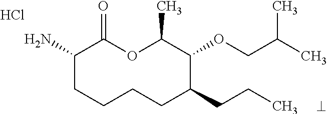 [SMILES: Cl ; CCC[C@H]1CCCC[C@H](N)C(=O)O[C@@H](C)[C@@H]1OCC(C)C] | Example 11, Step 1 | White Solid |
| 155 | 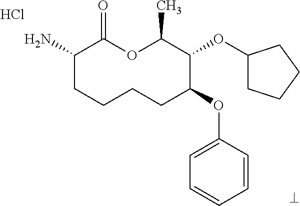 [SMILES: Cl ; C[C@@H]1OC(=O)[C@@H](N)CCCC[C@H](Oc2ccccc2)[C@H]1OC1CCCC1] | Example 11, Step 1 | White Solid |
| 156 | 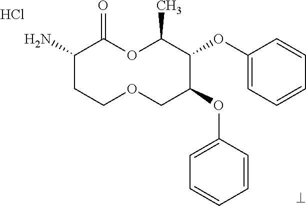 [SMILES: Cl ; C[C@@H]1OC(=O)[C@@H](N)CCOC[C@H](Oc2ccccc2)[C@H]1Oc1ccccc1] | Example 11, Step 1 | White Solid |
| 157 | 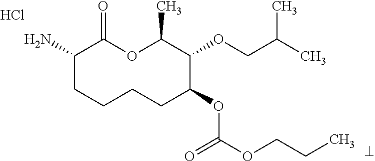 [SMILES: Cl ; CCCOC(=O)O[C@H]1CCCC[C@H](N)C(=O)O[C@@H](C)[C@@H]1OCC(C)C] | Example 11, Step 1 | White Solid |
| 158 | 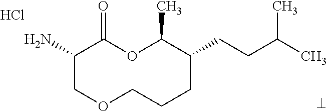 [SMILES: Cl ; CC(C)CC[C@@H]1CCCOC[C@H](N)C(=O)O[C@H]1C] | Example 11, Step 1 | White Powder |
| 159 | 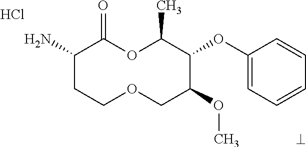 [SMILES: Cl ; CO[C@H]1COCC[C@H](N)C(=O)O[C@@H](C)[C@@H]1Oc1ccccc1] | Example 11, Step 1 | White Solid |
| 160 | 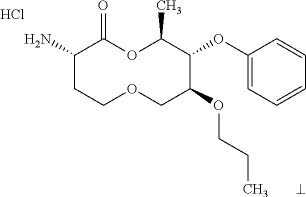 [SMILES: Cl ; CCCO[C@H]1COCC[C@H](N)C(=O)O[C@@H](C)[C@@H]1Oc1ccccc1] | Example 11, Step 1 | White Solid |
| 161 | 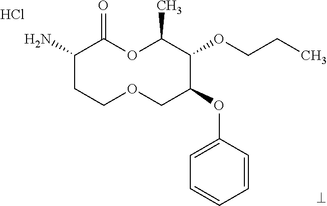 [SMILES: Cl ; CCCO[C@H]1[C@H](C)OC(=O)[C@@H](N)CCOC[C@@H]1Oc1ccccc1] | Example 11, Step 1 | White Solid |
| 162 | 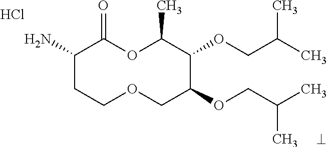 [SMILES: Cl ; CC(C)CO[C@H]1[C@H](C)OC(=O)[C@@H](N)CCOC[C@@H]1OCC(C)C] | Example 11, Step 1 | White Solid |
| 163 | 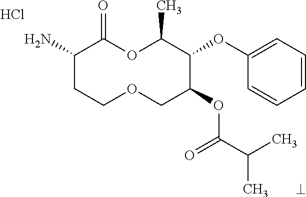 [SMILES: Cl ; CC(C)C(=O)O[C@H]1COCC[C@H](N)C(=O)O[C@@H](C)[C@@H]1Oc1ccccc1] | Example 11, Step 1 | White Solid |
| 164 | 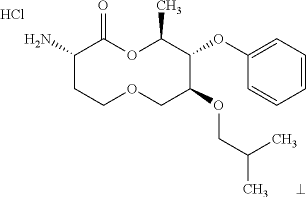 [SMILES: Cl ; CC(C)CO[C@H]1COCC[C@H](N)C(=O)O[C@@H](C)[C@@H]1Oc1ccccc1] | Example 11, Step 1 | White Solid |
| 165 | 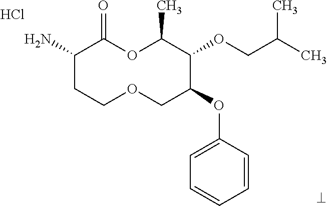 [SMILES: Cl ; CC(C)CO[C@H]1[C@H](C)OC(=O)[C@@H](N)CCOC[C@@H]1Oc1ccccc1] | Example 11, Step 1 | White Solid |
| 166 | 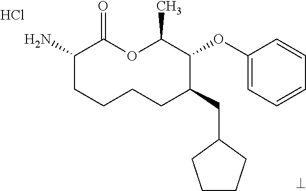 [SMILES: Cl ; C[C@@H]1OC(=O)[C@@H](N)CCCC[C@H](CC2CCCC2)[C@H]1Oc1ccccc1] | Example 11, Step 1 | White Solid |
| 167 | 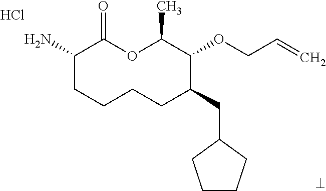 [SMILES: Cl ; C=CCO[C@@H]1[C@@H](CC2CCCC2)CCCC[C@H](N)C(=O)O[C@H]1C] | Example 11, Step 1 | White Solid |
| 168 | 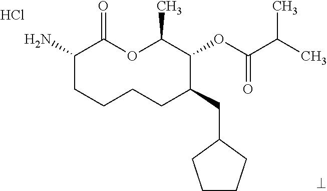 [SMILES: Cl ; CC(C)C(=O)O[C@@H]1[C@@H](CC2CCCC2)CCCC[C@H](N)C(=O)O[C@H]1C] | Example 11, Step 1 | White Solid |
| 169 | 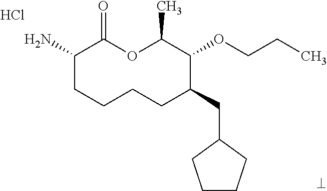 [SMILES: Cl ; CCCO[C@@H]1[C@@H](CC2CCCC2)CCCC[C@H](N)C(=O)O[C@H]1C] | Example 11, Step 1 | White Solid |
| 170 | 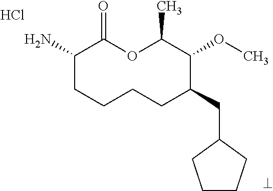 [SMILES: Cl ; CO[C@@H]1[C@@H](CC2CCCC2)CCCC[C@H](N)C(=O)O[C@H]1C] | Example 11, Step 1 | White Solid |
| 171 | 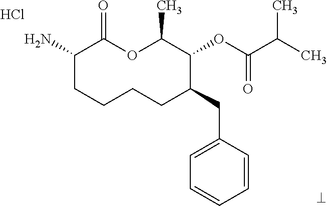 [SMILES: Cl ; CC(C)C(=O)O[C@@H]1[C@@H](Cc2ccccc2)CCCC[C@H](N)C(=O)O[C@H]1C] | Example 11, Step 1 | Oil |
| 172 | 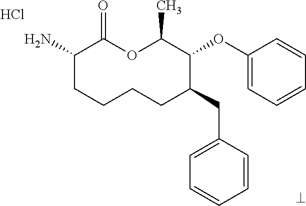 [SMILES: Cl ; C[C@@H]1OC(=O)[C@@H](N)CCCC[C@H](Cc2ccccc2)[C@H]1Oc1ccccc1] | Example 11, Step 1 | White Solid |
| 173 | 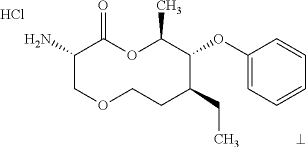 [SMILES: Cl ; CC[C@H]1CCOC[C@H](N)C(=O)O[C@@H](C)[C@@H]1Oc1ccccc1] | Example 11, Step 1 | White Solid |
| 174 | 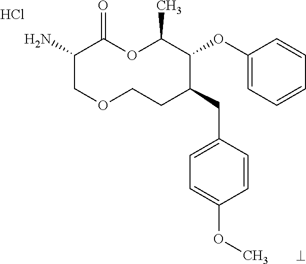 [SMILES: Cl ; COc1ccc(C[C@H]2CCOC[C@H](N)C(=O)O[C@@H](C)[C@@H]2Oc2ccccc2)cc1] | Example 11, Step 1 | Sticky Oil |
| 175 | 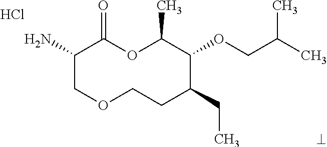 [SMILES: Cl ; CC[C@H]1CCOC[C@H](N)C(=O)O[C@@H](C)[C@@H]1OCC(C)C] | Example 11, Step 1 | White Solid |
| 176 | 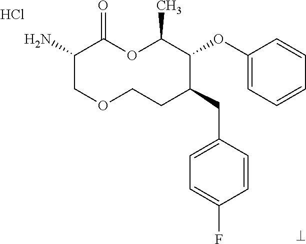 [SMILES: Cl ; C[C@@H]1OC(=O)[C@@H](N)COCC[C@H](Cc2ccc(F)cc2)[C@H]1Oc1ccccc1] | Example 11, Step 1 | Sticky Foam |
| 177 | 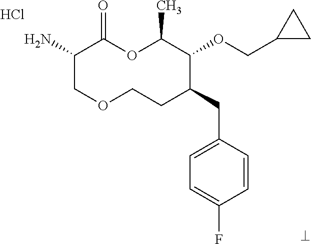 [SMILES: Cl ; C[C@@H]1OC(=O)[C@@H](N)COCC[C@H](Cc2ccc(F)cc2)[C@H]1OCC1CC1] | Example 11, Step 1 | White Solid |
| 178 | 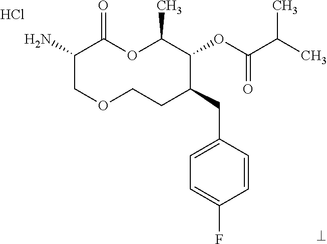 [SMILES: Cl ; CC(C)C(=O)O[C@@H]1[C@@H](Cc2ccc(F)cc2)CCOC[C@H](N)C(=O)O[C@H]1C] | Example 11, Step 1 | Oil |
| 179 | 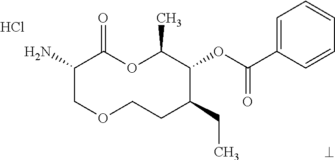 [SMILES: Cl ; CC[C@H]1CCOC[C@H](N)C(=O)O[C@@H](C)[C@@H]1OC(=O)c1ccccc1] | Example 11, Step 1 | White Solid |
| 180 | 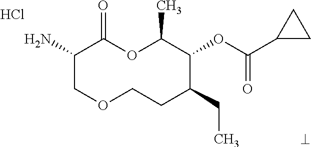 [SMILES: Cl ; CC[C@H]1CCOC[C@H](N)C(=O)O[C@@H](C)[C@@H]1OC(=O)C1CC1] | Example 11, Step 1 | White Solid |
| 181 | 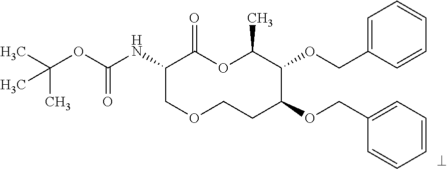 [SMILES: C[C@@H]1OC(=O)[C@@H](NC(=O)OC(C)(C)C)COCC[C@H](OCc2ccccc2)[C@H]1OCc1ccccc1] | Example 1, Steps 1-5 | White Foam |
| 182 | 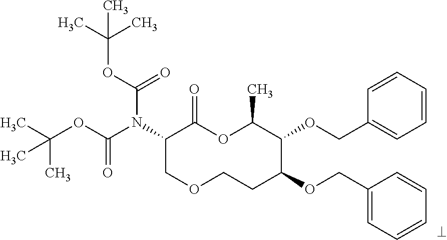 [SMILES: C[C@@H]1OC(=O)[C@@H](N(C(=O)OC(C)(C)C)C(=O)OC(C)(C)C)COCC[C@H](OCc2ccccc2)[C@H]1OCc1ccccc1] | Example 1, Step 1-6 | Colorless Oil |
| 183 | 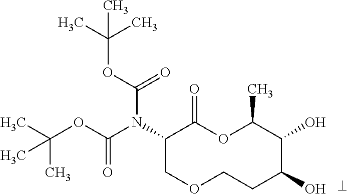 [SMILES: C[C@@H]1OC(=O)[C@@H](N(C(=O)OC(C)(C)C)C(=O)OC(C)(C)C)COCC[C@H](O)[C@H]1O] | Example 1, Steps 1-7 | Sticky White Solid |
| 184 | 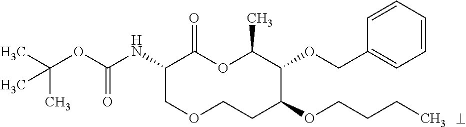 [SMILES: CCCCO[C@H]1CCOC[C@H](NC(=O)OC(C)(C)C)C(=O)O[C@@H](C)[C@@H]1OCc1ccccc1] | Example 2; Example 1, Steps 2-5 | Colorless Taffy |
| 185 | 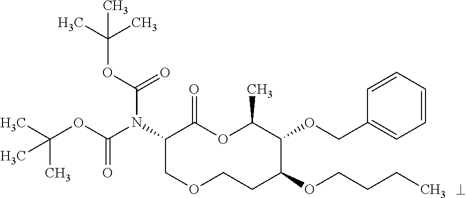 [SMILES: CCCCO[C@H]1CCOC[C@H](N(C(=O)OC(C)(C)C)C(=O)OC(C)(C)C)C(=O)O[C@@H](C)[C@@H]1OCc1ccccc1] | Example 2; Example 1, Steps 2-6 | Colorless Oil |
| 186 | 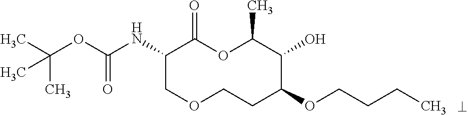 [SMILES: CCCCO[C@H]1CCOC[C@H](NC(=O)OC(C)(C)C)C(=O)O[C@@H](C)[C@@H]1O] | Example 2; Example 1, Steps 2-5, 7 | Sticky White Solid |
| 187 | 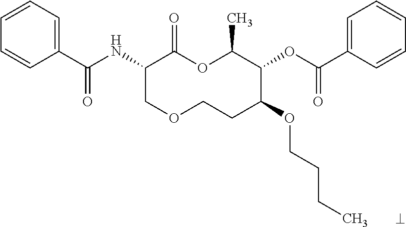 [SMILES: CCCCO[C@H]1CCOC[C@H](NC(=O)c2ccccc2)C(=O)O[C@@H](C)[C@@H]1OC(=O)c1ccccc1] | Example 2; Example 1, Steps 2-5, 7; Example 8B | Sticky White Solid |
| 188 | 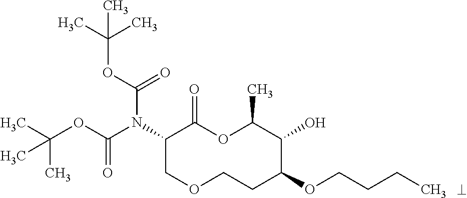 [SMILES: CCCCO[C@H]1CCOC[C@H](N(C(=O)OC(C)(C)C)C(=O)OC(C)(C)C)C(=O)O[C@@H](C)[C@@H]1O] | Example 2; Example 1, Steps 2-7 | Colorless Oil |
| 189 | 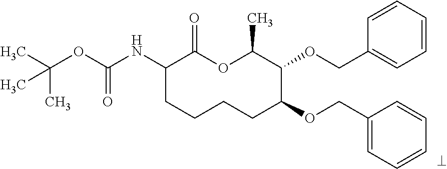 [SMILES: C[C@@H]1OC(=O)C(NC(=O)OC(C)(C)C)CCCC[C@H](OCc2ccccc2)[C@H]1OCc1ccccc1] | Example 7; Example 5, Step 4; Example 4, Step 4; Example 1, Step 5 | Clear Sticky Wax |
| 190 | 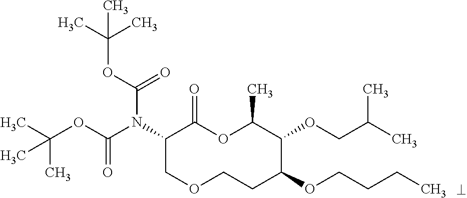 [SMILES: CCCCO[C@H]1CCOC[C@H](N(C(=O)OC(C)(C)C)C(=O)OC(C)(C)C)C(=O)O[C@@H](C)[C@@H]1OCC(C)C] | Example 2; Example 1, Steps 2-7; Example 8G | Colorless Oil |
| 191 | 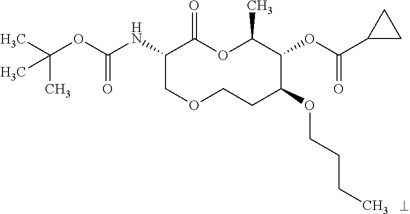 [SMILES: CCCCO[C@H]1CCOC[C@H](NC(=O)OC(C)(C)C)C(=O)O[C@@H](C)[C@@H]1OC(=O)C1CC1] | Example 2; Example 1, Steps 2-7; Example 8F | — |
| 192 | 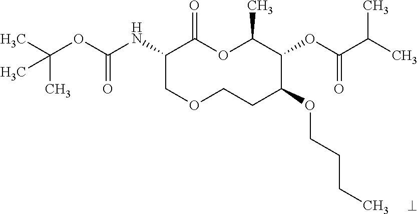 [SMILES: CCCCO[C@H]1CCOC[C@H](NC(=O)OC(C)(C)C)C(=O)O[C@@H](C)[C@@H]1OC(=O)C(C)C] | Example 2; Example 1, Steps 2-7; Example 8F | — |
| 193 | 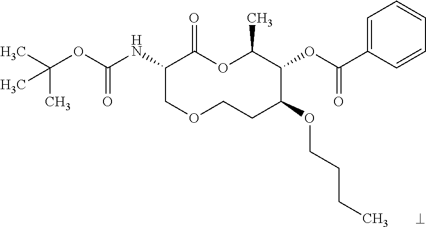 [SMILES: CCCCO[C@H]1CCOC[C@H](NC(=O)OC(C)(C)C)C(=O)O[C@@H](C)[C@@H]1OC(=O)c1ccccc1] | Example 2; Example 1, Steps 2-7; Example 8F | — |
| 194 | 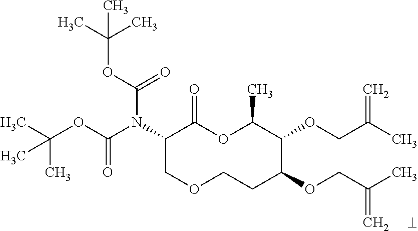 [SMILES: C=C(C)CO[C@H]1[C@H](C)OC(=O)[C@@H](N(C(=O)OC(C)(C)C)C(=O)OC(C)(C)C)COCC[C@@H]1OCC(=C)C] | Example 1, Steps 1-7; Example 8E, Step 1 | — |
| 195 | 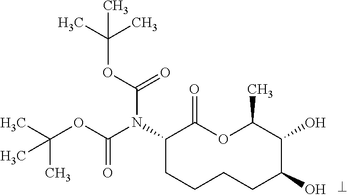 [SMILES: C[C@@H]1OC(=O)[C@@H](N(C(=O)OC(C)(C)C)C(=O)OC(C)(C)C)CCCC[C@H](O)[C@H]1O] | Example 7; Example 5, Step 4; Example 4, Step 4; Example 1, Steps 5-7 | — |
| 196 | 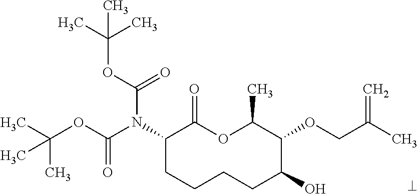 [SMILES: C=C(C)CO[C@H]1[C@H](C)OC(=O)[C@@H](N(C(=O)OC(C)(C)C)C(=O)OC(C)(C)C)CCCC[C@@H]1O] | Example 7; Example 5, Step 4; Example 4, Step 4; Example 1, Steps 5-7; Example 8A | Light Yellow Oil |
| 197 | 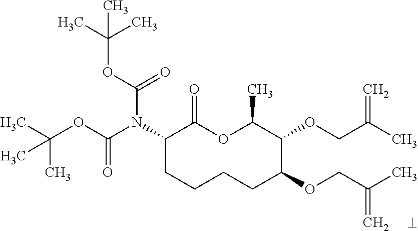 [SMILES: C=C(C)CO[C@H]1[C@H](C)OC(=O)[C@@H](N(C(=O)OC(C)(C)C)C(=O)OC(C)(C)C)CCCC[C@@H]1OCC(=C)C] | Example 7; Example 5, Step 4; Example 4, Step 4; Example 1, Steps 5-7; Example 8E, Step 1 | Light Yellow Oil |
| 198 | 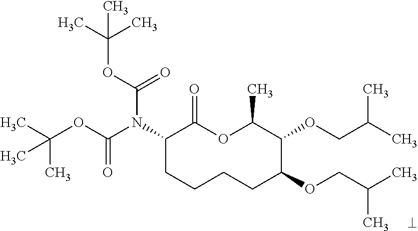 [SMILES: CC(C)CO[C@H]1[C@H](C)OC(=O)[C@@H](N(C(=O)OC(C)(C)C)C(=O)OC(C)(C)C)CCCC[C@@H]1OCC(C)C] | Example 7; Example 5, Step 4; Example 4, Step 4; Example 1, Steps 5-7; Example 8E | Light Yellow Oil |
| 199 | 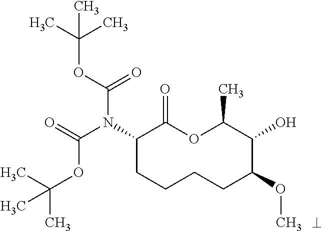 [SMILES: CO[C@H]1CCCC[C@H](N(C(=O)OC(C)(C)C)C(=O)OC(C)(C)C)C(=O)O[C@@H](C)[C@@H]1O] | Example 7; Example 5, Step 4; Example 4, Step 4; Example 1, Steps 5-7; Example 8I | Viscous Clear Oil |
| 200 | 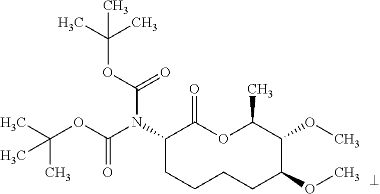 [SMILES: CO[C@H]1[C@H](C)OC(=O)[C@@H](N(C(=O)OC(C)(C)C)C(=O)OC(C)(C)C)CCCC[C@@H]1OC] | Example 7; Example 5, Step 4; Example 4, Step 4; Example 1, Steps 5-7; Example 8I | Viscous Clear Oil |
| 201 | 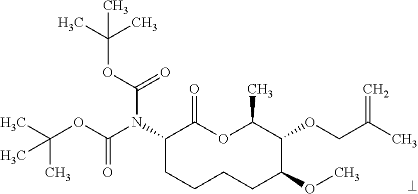 [SMILES: C=C(C)CO[C@H]1[C@H](C)OC(=O)[C@@H](N(C(=O)OC(C)(C)C)C(=O)OC(C)(C)C)CCCC[C@@H]1OC] | Example 7; Example 5, Step 4; Example 4, Step 4; Example 1, Steps 5-7; Example 8I; Example 8G, Step 1 | Colorless Clear Oil |
| 202 | 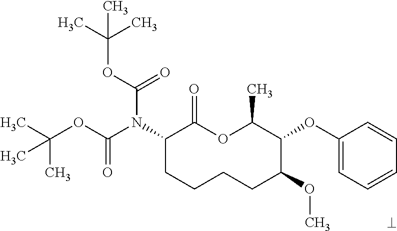 [SMILES: CO[C@H]1CCCC[C@H](N(C(=O)OC(C)(C)C)C(=O)OC(C)(C)C)C(=O)O[C@@H](C)[C@@H]1Oc1ccccc1] | Example 7; Example 5, Step 4; Example 4, Step 4; Example 1, Steps 5-7; Example 8C; Example 8H | Clear Colorless Oil |
| 203 | 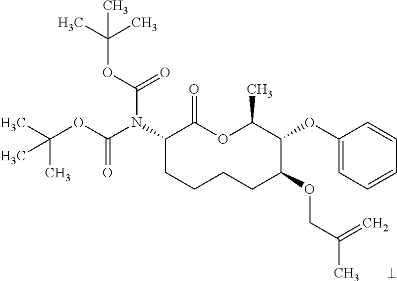 [SMILES: C=C(C)CO[C@H]1CCCC[C@H](N(C(=O)OC(C)(C)C)C(=O)OC(C)(C)C)C(=O)O[C@@H](C)[C@@H]1Oc1ccccc1] | Example 7; Example 5, Step 4; Example 4, Step 4; Example 1, Steps 5-7; Example 8C; Example 8G, Step 1 | Off White Oil |
| 204 | 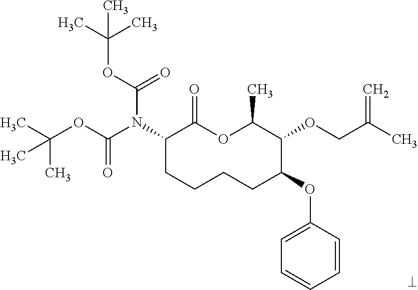 [SMILES: C=C(C)CO[C@H]1[C@H](C)OC(=O)[C@@H](N(C(=O)OC(C)(C)C)C(=O)OC(C)(C)C)CCCC[C@@H]1Oc1ccccc1] | Example 7; Example 5, Step 4; Example 4, Step 4; Example 1, Steps 5-7; Example 8A; Example 8C | — |
| 205 | 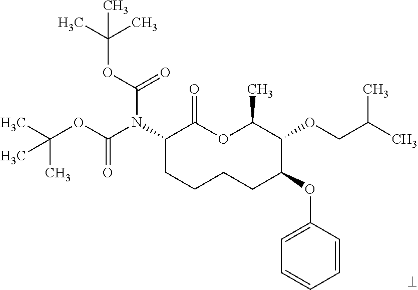 [SMILES: CC(C)CO[C@H]1[C@H](C)OC(=O)[C@@H](N(C(=O)OC(C)(C)C)C(=O)OC(C)(C)C)CCCC[C@@H]1Oc1ccccc1] | Example 7; Example 5, Step 4; Example 4, Step 4; Example 1, Steps 5-7; Example 8A; Example 8C; Example 8G, Step 2 | — |
| 206 | 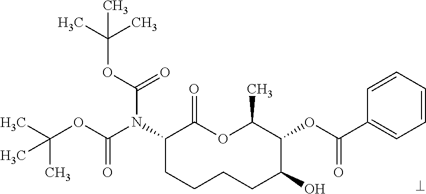 [SMILES: C[C@@H]1OC(=O)[C@@H](N(C(=O)OC(C)(C)C)C(=O)OC(C)(C)C)CCCC[C@H](O)[C@H]1OC(=O)c1ccccc1] | Example 7; Example 5, Step 4; Example 4, Step 4; Example 1, Steps 5-7; Example 8B | Clear Colorless Oil |
| 207 | 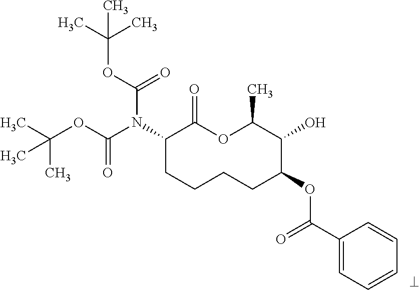 [SMILES: C[C@@H]1OC(=O)[C@@H](N(C(=O)OC(C)(C)C)C(=O)OC(C)(C)C)CCCC[C@H](OC(=O)c2ccccc2)[C@H]1O] | Example 7; Example 5, Step 4; Example 4, Step 4; Example 1, Steps 5-7; Example 8B | Sticky Solid |
| 208 | 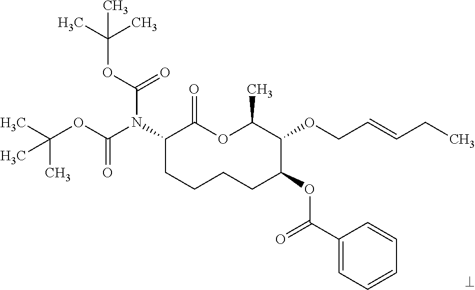 [SMILES: CC/C=C/CO[C@H]1[C@H](C)OC(=O)[C@@H](N(C(=O)OC(C)(C)C)C(=O)OC(C)(C)C)CCCC[C@@H]1OC(=O)c1ccccc1] | Example 7; Example 5, Step 4; Example 4, Step 4; Example 1, Steps 5-7; Example 8B; Example 8G Step 1 | Light Yellow Oil |
| 209 | 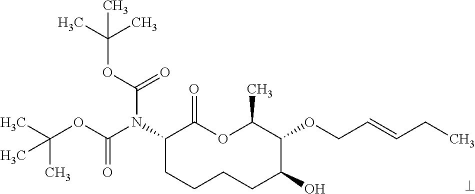 [SMILES: CC/C=C/CO[C@H]1[C@H](C)OC(=O)[C@@H](N(C(=O)OC(C)(C)C)C(=O)OC(C)(C)C)CCCC[C@@H]1O] | Example 7; Example 5, Step 4; Example 4, Step 4; Example 1, Steps 5-7; Example 8A | Yellow Oil |
| 210 | 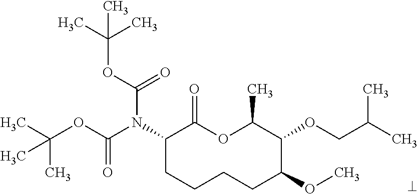 [SMILES: CO[C@H]1CCCC[C@H](N(C(=O)OC(C)(C)C)C(=O)OC(C)(C)C)C(=O)O[C@@H](C)[C@@H]1OCC(C)C] | Example 7; Example 5, Step 4; Example 4, Step 4; Example 1, Steps 5-7; Example 8I; Example 8G | Pale Yellow Solid |
| 211 | 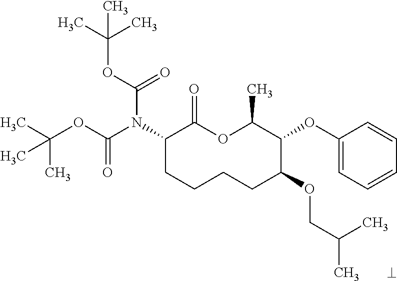 [SMILES: CC(C)CO[C@H]1CCCC[C@H](N(C(=O)OC(C)(C)C)C(=O)OC(C)(C)C)C(=O)O[C@@H](C)[C@@H]1Oc1ccccc1] | Example 7; Example 5, Step 4; Example 4, Step 4; Example 1, Steps 5-7; Example 8C; Example 8G | Light Yellow Oil |
| 212 | 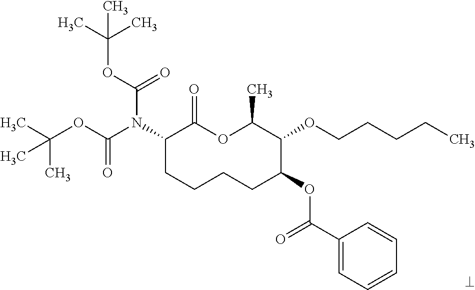 [SMILES: CCCCCO[C@H]1[C@H](C)OC(=O)[C@@H](N(C(=O)OC(C)(C)C)C(=O)OC(C)(C)C)CCCC[C@@H]1OC(=O)c1ccccc1] | Example 7; Example 5, Step 4; Example 4, Step 4; Example 1, Steps 5-7; Example 8B; Example 8G | Yellow Oil |
| 213 | 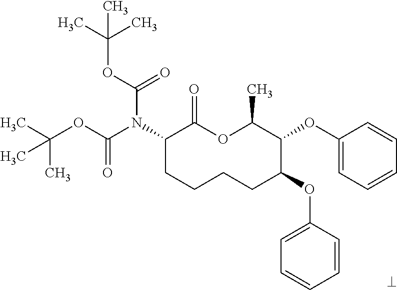 [SMILES: C[C@@H]1OC(=O)[C@@H](N(C(=O)OC(C)(C)C)C(=O)OC(C)(C)C)CCCC[C@H](Oc2ccccc2)[C@H]1Oc1ccccc1] | Example 7; Example 5, Step 4; Example 4, Step 4; Example 1, Steps 5-7; Example 8C | White Sticky Solid |
| 214 | 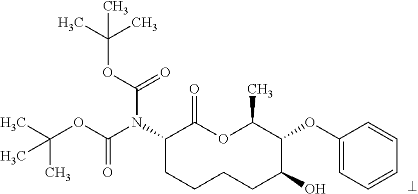 [SMILES: C[C@@H]1OC(=O)[C@@H](N(C(=O)OC(C)(C)C)C(=O)OC(C)(C)C)CCCC[C@H](O)[C@H]1Oc1ccccc1] | Example 7; Example 5, Step 4; Example 4, Step 4; Example 1, Steps 5-7; Example 8C | White Solid |
| 215 | 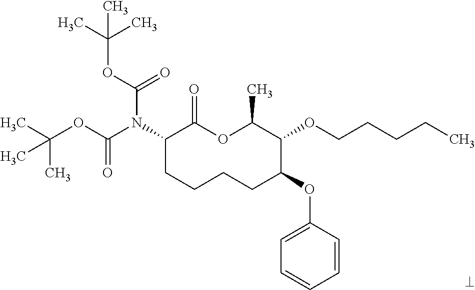 [SMILES: CCCCCO[C@H]1[C@H](C)OC(=O)[C@@H](N(C(=O)OC(C)(C)C)C(=O)OC(C)(C)C)CCCC[C@@H]1Oc1ccccc1] | Example 7; Example 5, Step 4; Example 4, Step 4; Example 1, Steps 5-7; Example 8A; Example 8G, Step 2; Example 8C | Yellow Oil |
| 216 | 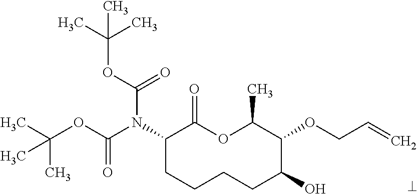 [SMILES: C=CCO[C@H]1[C@H](C)OC(=O)[C@@H](N(C(=O)OC(C)(C)C)C(=O)OC(C)(C)C)CCCC[C@@H]1O] | Example 7; Example 5, Step 4; Example 4, Step 4; Example 1, Steps 5-7; Example 8A | Yellow Sticky Oil |
| 217 | 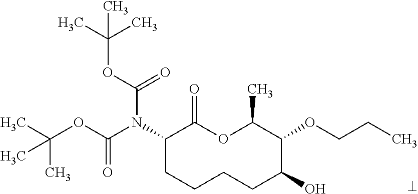 [SMILES: CCCO[C@H]1[C@H](C)OC(=O)[C@@H](N(C(=O)OC(C)(C)C)C(=O)OC(C)(C)C)CCCC[C@@H]1O] | Example 7; Example 5, Step 4; Example 4, Step 4; Example 1, Steps 5-7; Example 8A; Example 8G, Step 2 | Light Yellow Sticky Solid |
| 218 | 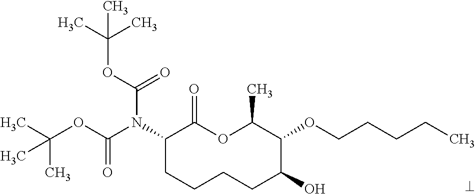 [SMILES: CCCCCO[C@H]1[C@H](C)OC(=O)[C@@H](N(C(=O)OC(C)(C)C)C(=O)OC(C)(C)C)CCCC[C@@H]1O] | Example 7; Example 5, Step 4; Example 4, Step 4; Example 1, Steps 5-7; Example 8A; Example 8G, Step 2 | — |
| 219 | 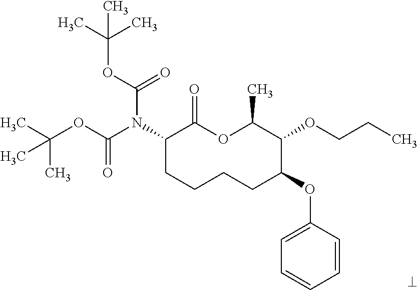 [SMILES: CCCO[C@H]1[C@H](C)OC(=O)[C@@H](N(C(=O)OC(C)(C)C)C(=O)OC(C)(C)C)CCCC[C@@H]1Oc1ccccc1] | Example 7; Example 5, Step 4; Example 4, Step 4; Example 1, Steps 5-7; Example 8A; Example 8G, Step 2; Example 8C | — |
| 221 | 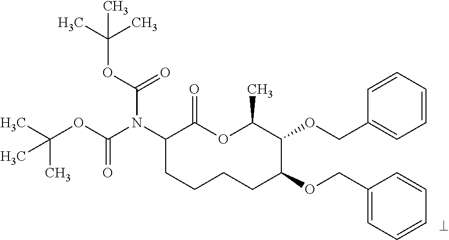 [SMILES: C[C@@H]1OC(=O)C(N(C(=O)OC(C)(C)C)C(=O)OC(C)(C)C)CCCC[C@H](OCc2ccccc2)[C@H]1OCc1ccccc1] | Example 7; Example 5, Step 4; Example 4, Step 4; Example 1, Steps 5, 6 | Clear Colorless Oil |
| 222 | 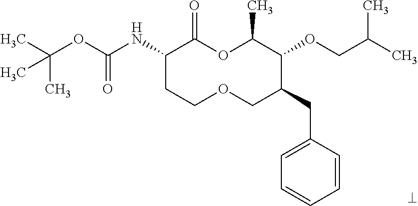 [SMILES: CC(C)CO[C@@H]1[C@@H](Cc2ccccc2)COCC[C@H](NC(=O)OC(C)(C)C)C(=O)O[C@H]1C] | Example 10, Steps 1-6 | Oil |
| 223 | 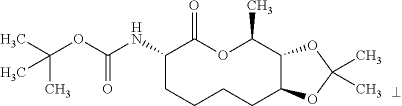 [SMILES: C[C@@H]1OC(=O)[C@@H](NC(=O)OC(C)(C)C)CCCC[C@@H]2OC(C)(C)O[C@@H]12] | Example 7; Example 5, Step 4; Example 4, Step 4; Example 1, Steps | White Solid |
| | | 5; | |
| | | Example | |
| | | 1, Step 7; | |
| | | Example | |
| | | 8J | |
| 224 | 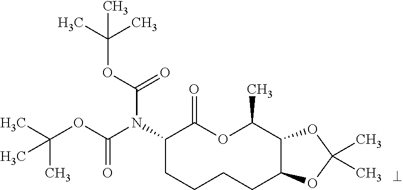 [SMILES: C[C@@H]1OC(=O)[C@@H](N(C(=O)OC(C)(C)C)C(=O)OC(C)(C)C)CCCC[C@@H]2OC(C)(C)O[C@@H]12] | Example 7; Example 5, Step 4; Example 4, Step 4; Example 1, Steps 5-7; Example 8J | Clear Sticky Oil |
| 225 | 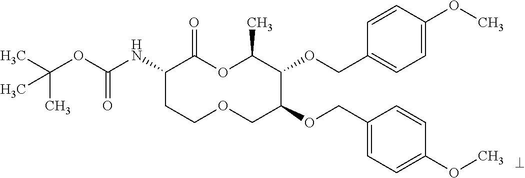 [SMILES: COc1ccc(CO[C@H]2[C@H](C)OC(=O)[C@@H](NC(=O)OC(C)(C)C)CCOC[C@@H]2OCc2ccc(OC)cc2)cc1] | Example 6, Steps 1-10 | Clear Colorless Oil |
| 226 | 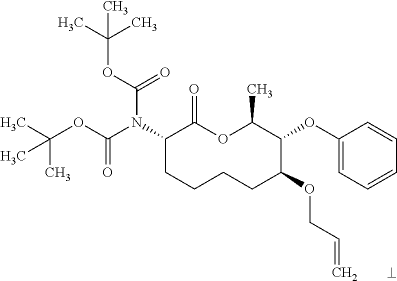 [SMILES: C=CCO[C@H]1CCCC[C@H](N(C(=O)OC(C)(C)C)C(=O)OC(C)(C)C)C(=O)O[C@@H](C)[C@@H]1Oc1ccccc1] | Example 7; Example 5, Step 4; Example 4, Step 4; Example 1, Steps 5-7; Example 8C; Example 8G, Step 1 | White Solid |
| 227 | 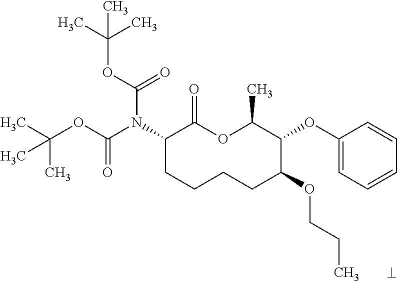 [SMILES: CCCO[C@H]1CCCC[C@H](N(C(=O)OC(C)(C)C)C(=O)OC(C)(C)C)C(=O)O[C@@H](C)[C@@H]1Oc1ccccc1] | Example 7; Example 5, Step 4; Example 4, Step 4; Example 1, Steps 5-7; Example 8C; Example 8G | Clear Sticky Oil |
| 228 | 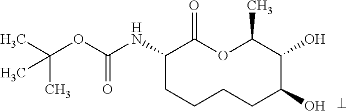 [SMILES: C[C@@H]1OC(=O)[C@@H](NC(=O)OC(C)(C)C)CCCC[C@H](O)[C@H]1O] | Example 7; Example 5, Step 4; Example 4, Step 4; Example 1, Step 5; | White Solid |
| | | Example | |
| | | 1, Step 7 | |
| 229 | 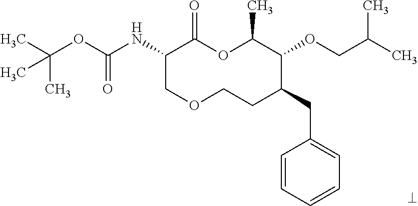 [SMILES: CC(C)CO[C@@H]1[C@@H](Cc2ccccc2)CCOC[C@H](NC(=O)OC(C)(C)C)C(=O)O[C@H]1C] | Example 3C; Example 4, Steps 1, 2A, 3A, 4; Example 1, Step 5 | Colorless Oil |
| 230 | 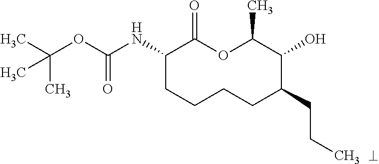 [SMILES: CCC[C@H]1CCCC[C@H](NC(=O)OC(C)(C)C)C(=O)O[C@@H](C)[C@@H]1O] | Example 3D; Example 5 | Whtie Solid |
| 231 | 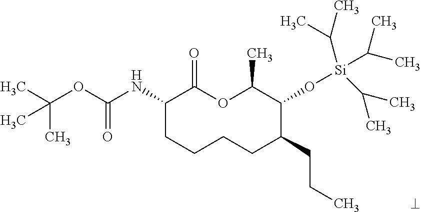 [SMILES: CCC[C@H]1CCCC[C@H](NC(=O)OC(C)(C)C)C(=O)O[C@@H](C)[C@@H]1O[Si](C(C)C)(C(C)C)C(C)C] | Example 3D; Example 5, Steps 1-6 | — |
| 232 | 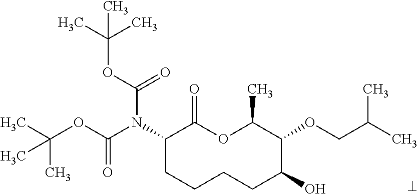 [SMILES: CC(C)CO[C@H]1[C@H](C)OC(=O)[C@@H](N(C(=O)OC(C)(C)C)C(=O)OC(C)(C)C)CCCC[C@@H]1O] | Example 7; Example 5, Step 4; Example 4, Step 4; Example 1, Steps 5-7; Example 8A; Example 8G, Step 2 | White Solid |
| 233 | 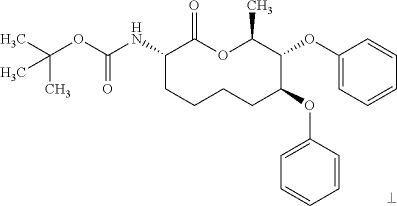 [SMILES: C[C@@H]1OC(=O)[C@@H](NC(=O)OC(C)(C)C)CCCC[C@H](Oc2ccccc2)[C@H]1Oc1ccccc1] | Example 7; Example 5, Step 4; Example 4, Step 4; Example 1, Step 5; Example 1, Step 7; Example 8C | White Solid |
| 234 | 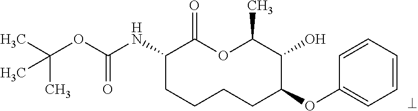 [SMILES: C[C@@H]1OC(=O)[C@@H](NC(=O)OC(C)(C)C)CCCC[C@H](Oc2ccccc2)[C@H]1O] | Example 7; Example 5, Step 4; Example 4, Step 4; Example 1, Step 5; | White Solid |
| | | Example | |
| | | 1, Step 7; | |
| | | Example | |
| | | 8C | |
| 235 | 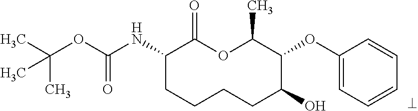 [SMILES: C[C@@H]1OC(=O)[C@@H](NC(=O)OC(C)(C)C)CCCC[C@H](O)[C@H]1Oc1ccccc1] | Example 7; Example 5, Step 4; Example 4, Step 4; Example 1, Step 5; | White Solid |
| | | Example | |
| | | 1, Step 7; | |
| | | Example | |
| | | 8C | |
| 236 | 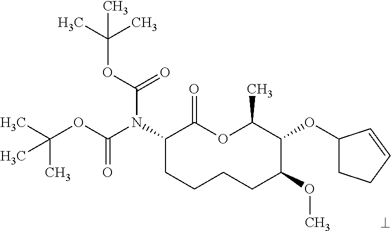 [SMILES: CO[C@H]1CCCC[C@H](N(C(=O)OC(C)(C)C)C(=O)OC(C)(C)C)C(=O)O[C@@H](C)[C@@H]1OC1C=CCC1] | Example 7; Example 5, Step 4; Example 4, Step 4; Example 1, Steps 5-7; Example 8I; Example 8G, Step 1 | Yellow Oil |
| 237 | 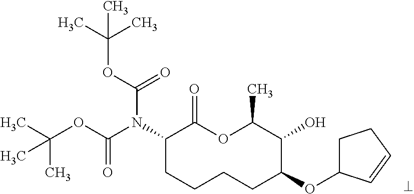 [SMILES: C[C@@H]1OC(=O)[C@@H](N(C(=O)OC(C)(C)C)C(=O)OC(C)(C)C)CCCC[C@H](OC2C=CCC2)[C@H]1O] | Example 7; Example 5, Step 4; Example 4, Step 4; Example 1, Steps 5-7; Example 8A | Yellow Solid |
| 238 | 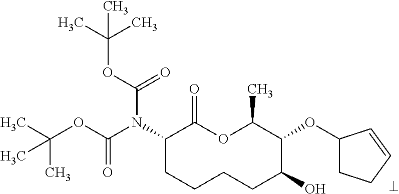 [SMILES: C[C@@H]1OC(=O)[C@@H](N(C(=O)OC(C)(C)C)C(=O)OC(C)(C)C)CCCC[C@H](O)[C@H]1OC1C=CCC1] | Example 7; Example 5, Step 4; Example 4, Step 4; Example 1, Steps 5-7; Example 8A | Yellow Solid |
| 239 | 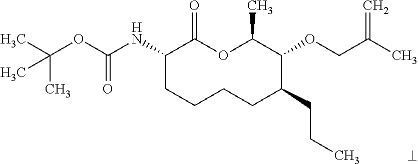 [SMILES: C=C(C)CO[C@@H]1[C@@H](CCC)CCCC[C@H](NC(=O)OC(C)(C)C)C(=O)O[C@H]1C] | Example 3D; Example 5; Example 8G, Step 1 | Colorless Oil |
| 240 | 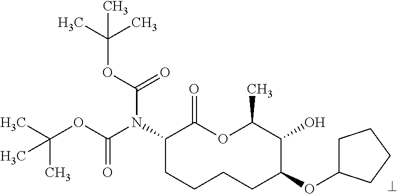 [SMILES: C[C@@H]1OC(=O)[C@@H](N(C(=O)OC(C)(C)C)C(=O)OC(C)(C)C)CCCC[C@H](OC2CCCC2)[C@H]1O] | Example 7; Example 5, Step 4; Example 4, Step 4; Example 1, Steps 5-7; Example 8A; Example 8G, Step 2 | White Solid |
| 241 | 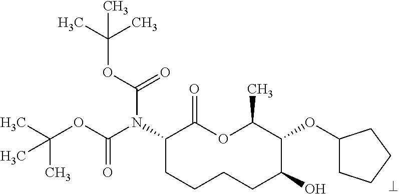 [SMILES: C[C@@H]1OC(=O)[C@@H](N(C(=O)OC(C)(C)C)C(=O)OC(C)(C)C)CCCC[C@H](O)[C@H]1OC1CCCC1] | Example 7; Example 5, Step 4; Example 4, Step 4; Example 1, Steps 5-7; Example 8A; Example 8G, Step 2 | White Solid |
| 242 | 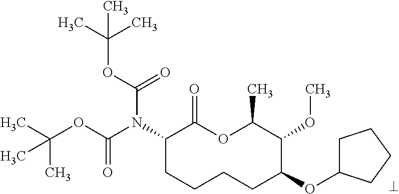 [SMILES: CO[C@H]1[C@H](C)OC(=O)[C@@H](N(C(=O)OC(C)(C)C)C(=O)OC(C)(C)C)CCCC[C@@H]1OC1CCCC1] | Example 7; Example 5, Step 4; Example 4, Step 4; Example 1, Steps 5-7; Example 8G; Example 8H | White Solid |
| 243 | 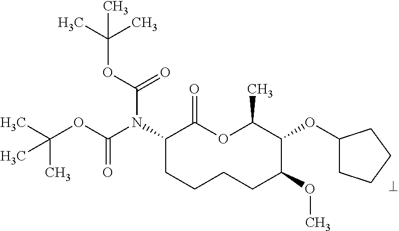 [SMILES: CO[C@H]1CCCC[C@H](N(C(=O)OC(C)(C)C)C(=O)OC(C)(C)C)C(=O)O[C@@H](C)[C@@H]1OC1CCCC1] | Example 7; Example 5, Step 4; Example 4, Step 4; Example 1, Steps 5-7; Example 8I; Example 8G | White Solid |
| 244 | 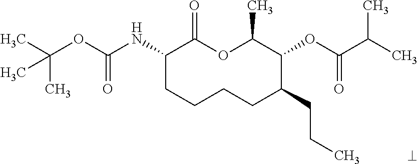 [SMILES: CCC[C@H]1CCCC[C@H](NC(=O)OC(C)(C)C)C(=O)O[C@@H](C)[C@@H]1OC(=O)C(C)C] | Example 3D; Example 5; Example 8F | Colorless Oil |
| 245 | 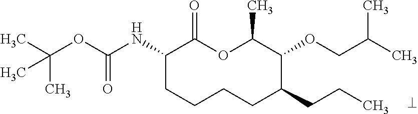 [SMILES: CCC[C@H]1CCCC[C@H](NC(=O)OC(C)(C)C)C(=O)O[C@@H](C)[C@@H]1OCC(C)C] | Example 3D; Example 5; Example 8G | Colorless Oil |
| 246 | 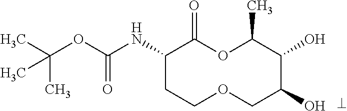 [SMILES: C[C@@H]1OC(=O)[C@@H](NC(=O)OC(C)(C)C)CCOC[C@H](O)[C@H]1O] | Example 6 | White Solid |
| 247 | 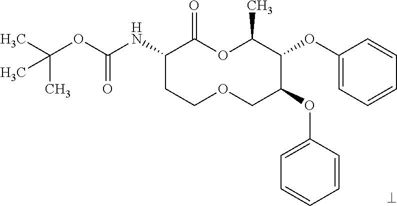 [SMILES: C[C@@H]1OC(=O)[C@@H](NC(=O)OC(C)(C)C)CCOC[C@H](Oc2ccccc2)[C@H]1Oc1ccccc1] | Example 6; Example 8C | White Solid |
| 248 | 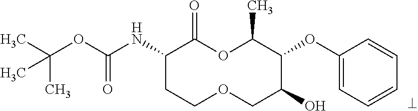 [SMILES: C[C@@H]1OC(=O)[C@@H](NC(=O)OC(C)(C)C)CCOC[C@H](O)[C@H]1Oc1ccccc1] | Example 6; Example 8C | White Solid |
| 249 | 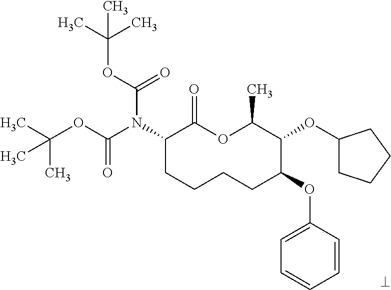 [SMILES: C[C@@H]1OC(=O)[C@@H](N(C(=O)OC(C)(C)C)C(=O)OC(C)(C)C)CCCC[C@H](Oc2ccccc2)[C@H]1OC1CCCC1] | Example 7; Example 5, Step 4; Example 4, Step 4; Example 1, Steps 5-7; Example 8A; Example 8G, Step 2; Example 8C | Sticky Wax |
| 250 | 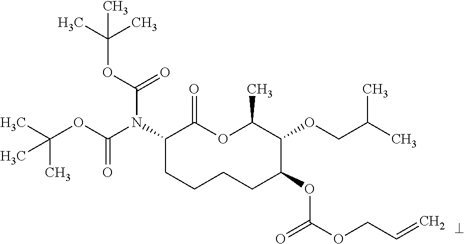 [SMILES: C=CCOC(=O)O[C@H]1CCCC[C@H](N(C(=O)OC(C)(C)C)C(=O)OC(C)(C)C)C(=O)O[C@@H](C)[C@@H]1OCC(C)C] | Example 7; Example 5, Step 4; Example 4, Step 4; Example 1, Steps 5-7; Example 8A; Example 8G, Step 2; Example 8G, Step 1 | Pale Yellow Oil |
| 251 | 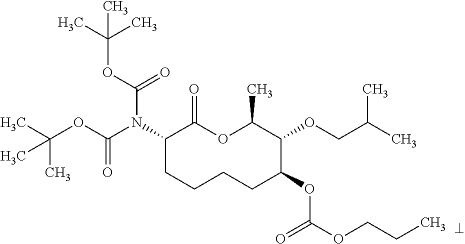 [SMILES: CCCOC(=O)O[C@H]1CCCC[C@H](N(C(=O)OC(C)(C)C)C(=O)OC(C)(C)C)C(=O)O[C@@H](C)[C@@H]1OCC(C)C] | Example 7; Example 5, Step 4; Example 4, Step 4; Example 1, Steps 5-7; Example 8A; Example 8G, Step 2; Example 8G | Clear Sticky Wax |
| 252 | 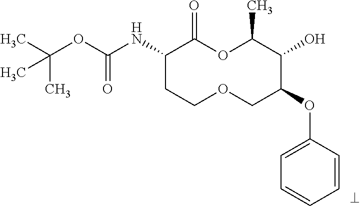 [SMILES: C[C@@H]1OC(=O)[C@@H](NC(=O)OC(C)(C)C)CCOC[C@H](Oc2ccccc2)[C@H]1O] | Example 6; Example 8C | White Foam |
| 253 | 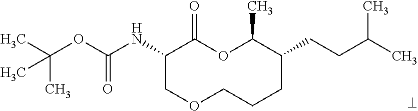 [SMILES: CC(C)CC[C@@H]1CCCOC[C@H](NC(=O)OC(C)(C)C)C(=O)O[C@H]1C] | Example 9; Steps 1-9 | Colorless Oil |
| 254 | 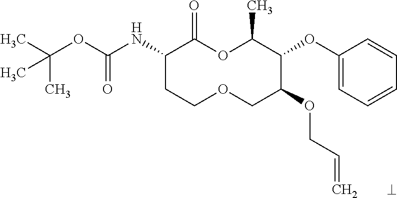 [SMILES: C=CCO[C@H]1COCC[C@H](NC(=O)OC(C)(C)C)C(=O)O[C@@H](C)[C@@H]1Oc1ccccc1] | Example 6; Example 8C; Example 8A | Sticky Wax |
| 255 | 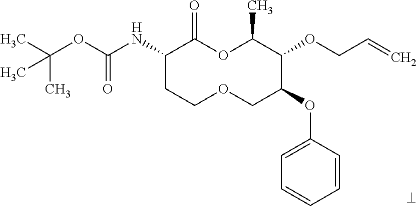 [SMILES: C=CCO[C@H]1[C@H](C)OC(=O)[C@@H](NC(=O)OC(C)(C)C)CCOC[C@@H]1Oc1ccccc1] | Example 6; Example 8A; Example 8C | Colorless Sticky Wax |
| 256 | 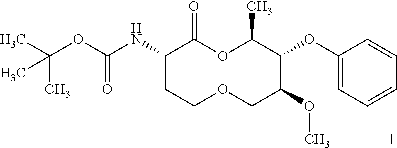 [SMILES: CO[C@H]1COCC[C@H](NC(=O)OC(C)(C)C)C(=O)O[C@@H](C)[C@@H]1Oc1ccccc1] | Example 6; Example 8C; Example 8H | White Solid |
| 257 | 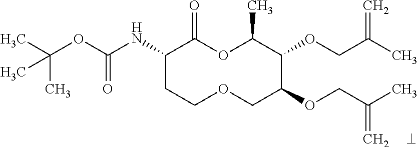 [SMILES: C=C(C)CO[C@H]1[C@H](C)OC(=O)[C@@H](NC(=O)OC(C)(C)C)CCOC[C@@H]1OCC(=C)C] | Example 6; Example 8D | Sticky Yellow Wax |
| 258 | 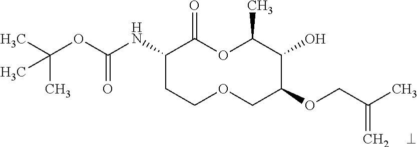 [SMILES: C=C(C)CO[C@H]1COCC[C@H](NC(=O)OC(C)(C)C)C(=O)O[C@@H](C)[C@@H]1O] | Example 6; Example 8A | Sticky Wax |
| 259 | 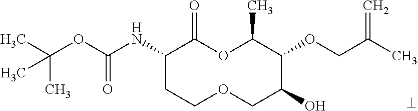 [SMILES: C=C(C)CO[C@H]1[C@H](C)OC(=O)[C@@H](NC(=O)OC(C)(C)C)CCOC[C@@H]1O] | Example 6; Example 8A | Sticky Wax |
| 260 | 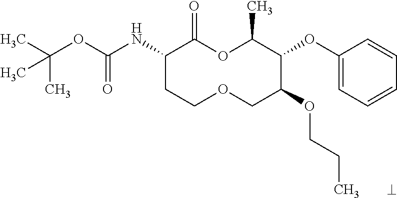 [SMILES: CCCO[C@H]1COCC[C@H](NC(=O)OC(C)(C)C)C(=O)O[C@@H](C)[C@@H]1Oc1ccccc1] | Example 6; Example 8C; Example 8A; Example 8G, Step 2 | White Solid |
| 261 | 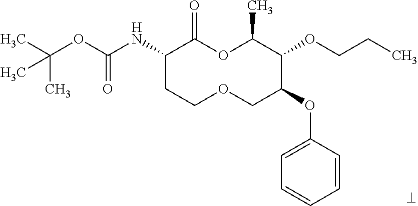 [SMILES: CCCO[C@H]1[C@H](C)OC(=O)[C@@H](NC(=O)OC(C)(C)C)CCOC[C@@H]1Oc1ccccc1] | Example 6; Example 8A; Example 8G, Step 2; Example 8C | White Semi Solid |
| 262 | 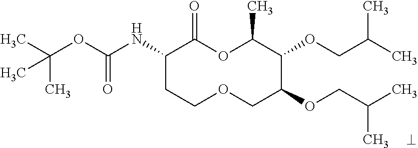 [SMILES: CC(C)CO[C@H]1[C@H](C)OC(=O)[C@@H](NC(=O)OC(C)(C)C)CCOC[C@@H]1OCC(C)C] | Example 6; Example 8D; Example 8E, Example 8G, Step 2 | White Solid |
| 263 | 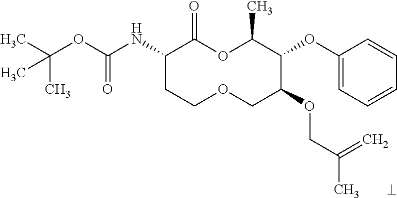 [SMILES: C=C(C)CO[C@H]1COCC[C@H](NC(=O)OC(C)(C)C)C(=O)O[C@@H](C)[C@@H]1Oc1ccccc1] | Example 6; Example 8C; Example 8A; | White Solid |
| 264 | 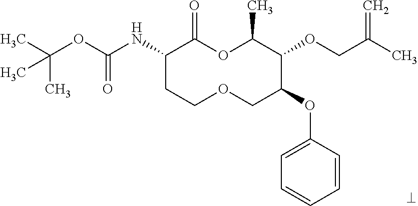 [SMILES: C=C(C)CO[C@H]1[C@H](C)OC(=O)[C@@H](NC(=O)OC(C)(C)C)CCOC[C@@H]1Oc1ccccc1] | Example 6; Example 8A; Example 8C | Sticky Wax |
| 265 | 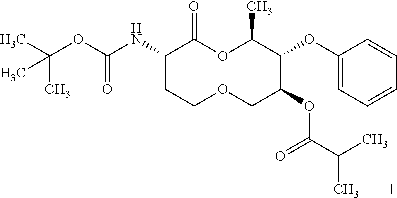 [SMILES: CC(C)C(=O)O[C@H]1COCC[C@H](NC(=O)OC(C)(C)C)C(=O)O[C@@H](C)[C@@H]1Oc1ccccc1] | Example 6; Example 8C; Example 8F | White Solid |
| 266 | 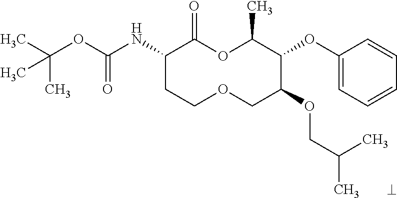 [SMILES: CC(C)CO[C@H]1COCC[C@H](NC(=O)OC(C)(C)C)C(=O)O[C@@H](C)[C@@H]1Oc1ccccc1] | Example 6; Example 8C; Example 8A; Example 8G, Step 2 | White Solid |
| 267 | 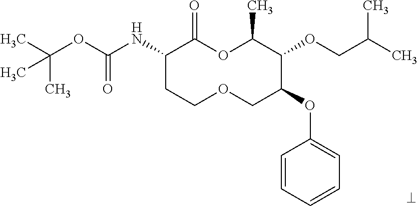 [SMILES: CC(C)CO[C@H]1[C@H](C)OC(=O)[C@@H](NC(=O)OC(C)(C)C)CCOC[C@@H]1Oc1ccccc1] | Example 6; Example 8A; Example 8C; Example 8G, Step 2 | Sticky Wax |
| 268 | 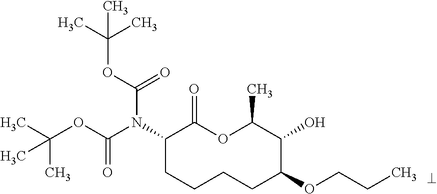 [SMILES: CCCO[C@H]1CCCC[C@H](N(C(=O)OC(C)(C)C)C(=O)OC(C)(C)C)C(=O)O[C@@H](C)[C@@H]1O] | Example 7; Example 5, Step 4; Example 4, Step 4; Example 1, Steps 5-7; Example 8A Example 8G, Step 2 | Sticky Wax |
| 269 | 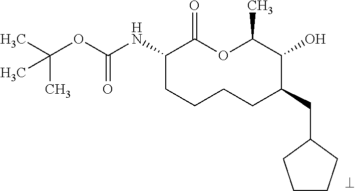 [SMILES: C[C@@H]1OC(=O)[C@@H](NC(=O)OC(C)(C)C)CCCC[C@H](CC2CCCC2)[C@H]1O] | Example 3D; Example 4, Step 1; Example 5 | White Solid |
| 270 | 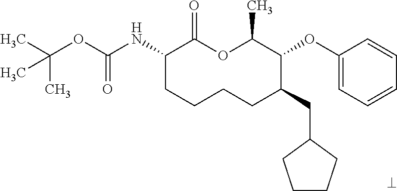 [SMILES: C[C@@H]1OC(=O)[C@@H](NC(=O)OC(C)(C)C)CCCC[C@H](CC2CCCC2)[C@H]1Oc1ccccc1] | Example 3D; Example 4, Step 1; Example 5; Example 8C | White Solid |
| 271 | 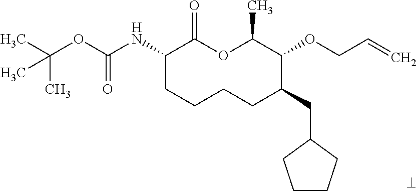 [SMILES: C=CCO[C@@H]1[C@@H](CC2CCCC2)CCCC[C@H](NC(=O)OC(C)(C)C)C(=O)O[C@H]1C] | Example 3D; Example 4, Step 1; Example 5; Example 8G, Step 1 | Colorless Oil |
| 272 | 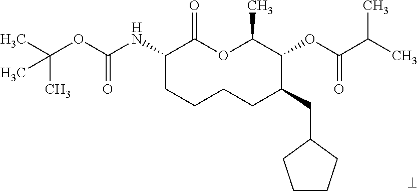 [SMILES: CC(C)C(=O)O[C@@H]1[C@@H](CC2CCCC2)CCCC[C@H](NC(=O)OC(C)(C)C)C(=O)O[C@H]1C] | Example 3D; Example 4, Step 1; Example 5; Example 8F | White Solid |
| 273 | 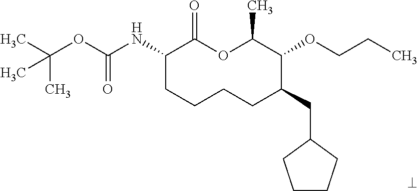 [SMILES: CCCO[C@@H]1[C@@H](CC2CCCC2)CCCC[C@H](NC(=O)OC(C)(C)C)C(=O)O[C@H]1C] | Example 3D; Example 4, Step 1; Example 5; Example 8G | Colorless Oil |
| 274 | 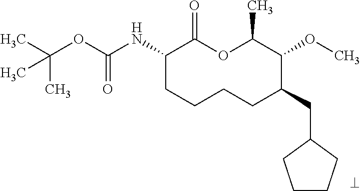 [SMILES: CO[C@@H]1[C@@H](CC2CCCC2)CCCC[C@H](NC(=O)OC(C)(C)C)C(=O)O[C@H]1C] | Example 3D; Example 4, Step 1; Example 5; Example 8H | Colorless Oil |
| 275 | 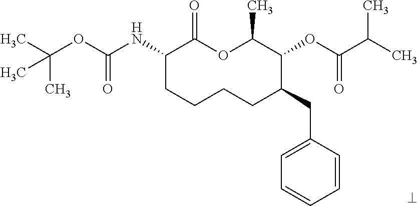 [SMILES: CC(C)C(=O)O[C@@H]1[C@@H](Cc2ccccc2)CCCC[C@H](NC(=O)OC(C)(C)C)C(=O)O[C@H]1C] | Example 3D; Example 4, Step 1; Example 5; Example 8F | White Solid |
| 276 | 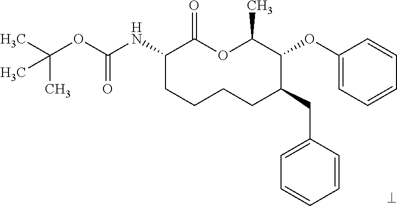 [SMILES: C[C@@H]1OC(=O)[C@@H](NC(=O)OC(C)(C)C)CCCC[C@H](Cc2ccccc2)[C@H]1Oc1ccccc1] | Example 3D; Example 4, Step 1; Example 5; Example 8C | Hard White Foam |
| 277 | 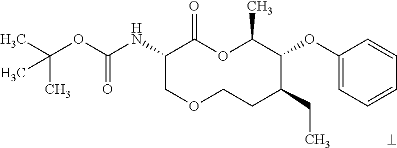 [SMILES: CC[C@H]1CCOC[C@H](NC(=O)OC(C)(C)C)C(=O)O[C@@H](C)[C@@H]1Oc1ccccc1] | Example 3A; Example 4, Steps 1, 2A, 3A, 4; Example 1, Step 5 | White Solid |
| 278 | 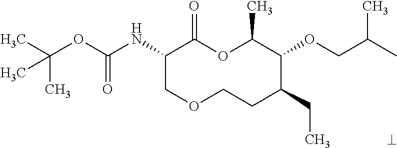 [SMILES: CC[C@H]1CCOC[C@H](NC(=O)OC(C)(C)C)C(=O)O[C@@H](C)[C@@H]1OCC(C)C] | Example 3C; Example 4, Steps 1, 2A, 3A, 4; Example 1, Step 5 | Colorless Semi Solid |
| 279 | 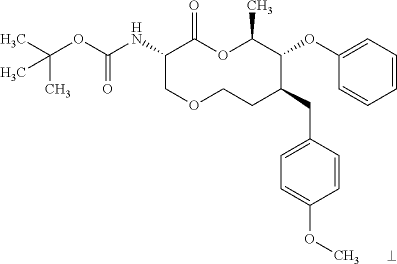 [SMILES: COc1ccc(C[C@H]2CCOC[C@H](NC(=O)OC(C)(C)C)C(=O)O[C@@H](C)[C@@H]2Oc2ccccc2)cc1] | Example 3A; Example 4, Steps 1, 2A, 3A, 4; Example 1, Step 5 | White Solid |
| 280 | 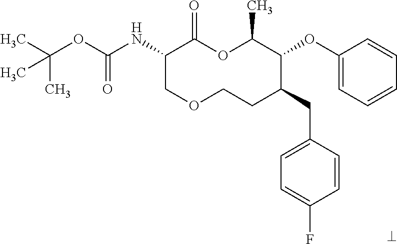 [SMILES: C[C@@H]1OC(=O)[C@@H](NC(=O)OC(C)(C)C)COCC[C@H](Cc2ccc(F)cc2)[C@H]1Oc1ccccc1] | Example 3A; Example 4, Steps 1, 2A, 3A, 4; Example 1, Step 5 | White Solid |
| 281 | 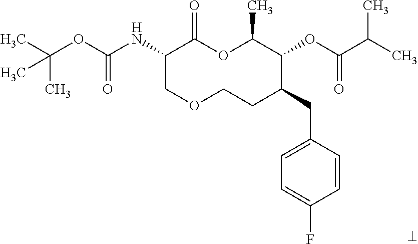 [SMILES: CC(C)C(=O)O[C@@H]1[C@@H](Cc2ccc(F)cc2)CCOC[C@H](NC(=O)OC(C)(C)C)C(=O)O[C@H]1C] | Example 3B; Example 4, Steps 1, 2B, 3B; Example 1, Step 5 | White Solid |
| 282 | 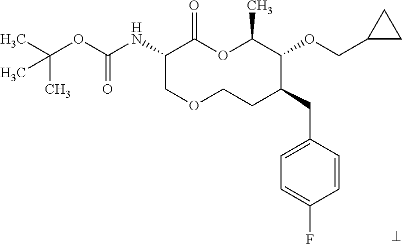 [SMILES: C[C@@H]1OC(=O)[C@@H](NC(=O)OC(C)(C)C)COCC[C@H](Cc2ccc(F)cc2)[C@H]1OCC1CC1] | Example 3C; Example 4, Steps 1, 2A, 3A, 4; Example 1, Step 5 | Colorless Semi Solid |
| 283 | 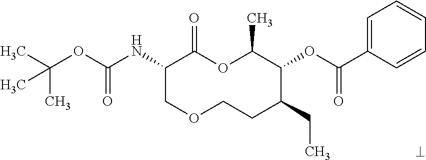 [SMILES: CC[C@H]1CCOC[C@H](NC(=O)OC(C)(C)C)C(=O)O[C@@H](C)[C@@H]1OC(=O)c1ccccc1] | Example 3B; Example 4, Steps 1, 2B, 3B; Example 1, Step 5 | White Solid |
| 284 | 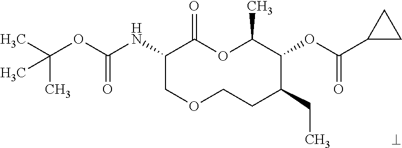 [SMILES: CC[C@H]1CCOC[C@H](NC(=O)OC(C)(C)C)C(=O)O[C@@H](C)[C@@H]1OC(=O)C1CC1] | Example 3B; Example 4, Steps 1, 2B, 3B; Example 1, Step 5 | Colorless Semi Solid |
| 285 | 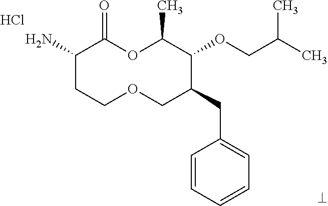 [SMILES: Cl ; CC(C)CO[C@@H]1[C@@H](Cc2ccccc2)COCC[C@H](N)C(=O)O[C@H]1C] | Example 11, Step 1 | Brown Foam |

TABLE 2
Analytical Data

| Cmpd | MP | IR | | NMR |
|---|---|---|---|---|
| No. | (° C.) | (cm⁻¹) | MASS | (¹H, ¹³C, ¹⁹F) |
| 1 | — | — | HRMS-ESI | ¹H NMR (400 MHz, CDCl₃) δ 8.71 (d, J = 8.1 Hz, |
| | | | (m/z) | 1H), 8.31 (d, J = 5.4 Hz, 1H), |
| | | | [M + H]⁺ | 7.41-7.20 (m, 10H), 6.95 (d, J = 5.4 Hz, 1H), 5.76 (d, J = 6.4 Hz, |
| | | | calcd for | 1H), 5.73 (d, J = 6.4 Hz, 1H), |
| | | | C₃₃H₃₉N₂O₁₀, | 5.32 (dq, J = 8.9, 6.3 Hz, 1H), 5.03 (d, J = 11.2 Hz, |
| | | | 623.2599; | 1H), 4.97-4.88 (m, 2H), 4.58 (d, J = 11.2 Hz, |
| | | | found, | 1H), 4.54 (d, J = 11.4 Hz, 1H), 3.90 (s, |
| | | | 623.2612 | 3H), 3.89 (d, J = 2.0 Hz, 2H), 3.70-3.57 (m, |
| | | | | 2H), 3.39 (dt, J = 12.1, 2.9 Hz, 1H), 3.32 (t, J = 8.9 Hz, |
| | | | | 1H), 2.19-2.07 (m, 1H), 2.06 (s, |
| | | | | 3H), 1.66-1.51 (m, 1H), 1.34 (d, J = 6.3 Hz, |
| | | | | 3H) |
| | | | | ¹³C NMR (101 MHz, CDCl₃) δ 170.24, |
| | | | | 169.28, 163.37, 160.26, 145.86, 143.96, |
| | | | | 142.40, 138.86, 138.15, 128.38, 128.31, |
| | | | | 128.10, 127.93, 127.76, 127.53, 109.66, |
| | | | | 89.54, 84.61, 78.10, 75.24, 75.19, 73.24, |
| | | | | 67.17, 66.89, 56.18, 54.00, 34.30, 20.87, |
| | | | | 19.02 |
| 2 | — | — | HRMS-ESI | ¹H NMR (400 MHz, CDCl₃) δ 8.73 (d, J = 7.9 Hz, |
| | | | (m/z) | 1H), 8.31 (d, J = 5.4 Hz, 1H), |
| | | | [M + H]⁺ | 7.41-7.27 (m, 5H), 6.95 (d, J = 5.5 Hz, 1H), 5.76 (d, J = 6.4 Hz, |
| | | | calcd for | 1H), 5.73 (d, J = 6.4 Hz, 1H), |
| | | | C₃₀H₄₁N₂O₁₀, | 5.25 (dq, J = 8.8, 6.3 Hz, 1H), 5.01 (d, J = 11.3 Hz, |
| | | | 589.2756; | 1H), 4.97-4.89 (m, 1H), 4.55 (d, J = 11.3 Hz, |
| | | | found, | 1H), 3.97-3.90 (m, 5H), 3.86 (ddd, J = 9.4, |
| | | | 589.2773 | 7.1, 6.2 Hz, 1H), 3.81-3.71 (m, 1H), |
| | | | | 3.45 (dq, J = 10.2, 7.0 Hz, 3H), 3.23 (t, J = 8.9 Hz, |
| | | | | 1H), 2.15-2.07 (m, 1H), 2.07 (s, |
| | | | | 3H), 1.57-1.42 (m, 3H), 1.38-1.32 (m, |
| | | | | 1H), 1.31 (d, J = 6.3 Hz, 3H), 1.30-1.25 (m, |
| | | | | 1H), 0.87 (t, J = 7.4 Hz, 3H) |
| | | | | ¹³C NMR (101 MHz, CDCl₃) δ 170.27, |
| | | | | 169.24, 163.38, 160.23, 145.87, 143.93, |
| | | | | 142.41, 138.35, 128.35, 127.96, 127.66, |
| | | | | 109.61, 89.54, 84.44, 78.29, 75.03, 73.25, |
| | | | | 73.09, 67.21, 67.13, 56.17, 54.03, 34.35, |
| | | | | 32.51, 20.88, 19.28, 18.98, 13.96 |
| 3 | — | — | HRMS-ESI | ¹H NMR (400 MHz, CDCl₃) δ 8.72 (d, J = 8.1 Hz, |
| | | | (m/z) | 1H), 8.31 (d, J = 5.4 Hz, 1H), 6.97 (d, J = 5.4 Hz, |
| | | | [M + H]⁺ | 1H), 5.89-5.65 (m, 2H), 5.37 (dq, J = 8.9, |
| | | | calcd for | 6.3 Hz, 1H), 4.99 (d, J = 8.1 Hz, 1H), |
| | | | C₂₇H₄₁N₂O₁₁, | 4.78 (t, J = 8.7 Hz, 1H), 3.93 (s, 2H), 3.92 (s, |
| | | | 569.2705; | 3H), 3.77 (dd, J = 11.8, 10.0 Hz, 1H), |
| | | | found, | 3.58 (dt, J = 9.1, 6.6 Hz, 1H), 3.52-3.36 (m, 3H), |
| | | | 569.2728 | 2.60 (hept, J = 7.0 Hz, 1H), 2.23-2.11 (m, |
| | | | | 1H), 2.07 (s, 3H), 1.62 (dt, J = 15.7, 3.0 Hz, |
| | | | | 1H), 1.56-1.38 (m, 2H), 1.38-1.27 (m, |
| | | | | 5H), 1.21 (t, J = 7.1 Hz, 6H), 0.89 (t, J = 7.3 Hz, |
| | | | | 3H) |
| | | | | ¹³C NMR (101 MHz, CDCl₃) δ 175.99, |
| | | | | 170.23, 169.35, 163.36, 160.25, 145.85, |
| | | | | 143.98, 142.28, 109.69, 89.48, 75.66, 75.58, |
| | | | | 72.65, 72.08, 67.81, 67.54, 56.19, 53.93, |
| | | | | 34.01, 33.82, 32.15, 20.85, 19.24, 19.19, |
| | | | | 18.60, 13.89 |
| 4 | — | — | HRMS-ESI | ¹H NMR (400 MHz, CDCl₃) δ 8.75 (d, J = 8.1 Hz, |
| | | | (m/z) | 1H), 8.32 (d, J = 5.4 Hz, 1H), 8.09 (dd, J = 8.1, |
| | | | [M + H]⁺ | 1.0 Hz, 2H), 7.65-7.54 (m, 1H), |
| | | | calcd for | 7.47 (t, J = 7.7 Hz, 2H), 6.97 (d, J = 5.4 Hz, 1H), |
| | | | C₃₀H₃₉N₂O₁₁, | 5.84-5.69 (m, 2H), 5.56 (dq, J = 8.6, 6.4 Hz, |
| | | | 603.2548; | 1H), 5.11-5.05 (m, 1H), 5.03 (dd, J = 8.1, |
| | | | found, | 2.3 Hz, 1H), 4.03-3.97 (m, 1H), |
| | | | 603.2544 | 3.97-3.93 (m, 1H), 3.92 (s, 3H), 3.79 (dd, J = 12.0, 10.2 Hz, |
| | | | | 1H), 3.56-3.47 (m, 3H), 3.42 (dt, J = 9.0, |
| | | | | 6.6 Hz, 1H), 2.35-2.17 (m, 1H), 2.08 (s, |
| | | | | 3H), 1.66 (dd, J = 5.9, 2.8 Hz, 1H), 1.40 (d, J = 6.4 Hz, |
| | | | | 3H), 1.34-1.22 (m, 1H), |
| | | | | 1.22-1.13 (m, 1H), 1.08 (dt, J = 14.5, 7.2 Hz, 2H), |
| | | | | 0.66 (t, J = 7.3 Hz, 3H) |
| | | | | ¹³C NMR (101 MHz, CDCl₃) δ 170.36, |
| | | | | 169.56, 165.66, 163.49, 160.37, 145.95, |
| | | | | 144.13, 142.41, 133.25, 129.93, 129.83, |
| | | | | 128.52, 109.79, 89.62, 76.71, 76.16, 73.15, |
| | | | | 72.15, 67.74, 67.51, 56.30, 54.09, 33.87, |
| | | | | 32.11, 20.97, 19.08, 18.85, 13.80 |
| 5 | — | — | HRMS-ESI | ¹H NMR (400 MHz, CDCl₃) δ 8.72 (d, J = 5.7 Hz, |
| | | | (m/z) | 1H), 8.31 (dd, J = 5.2, 3.1 Hz, 1H), |
| | | | [M + H]⁺ | 6.97 (dd, J = 5.4, 1.5 Hz, 1H), 5.85-5.64 (m, 2H), |
| | | | calcd for | 5.47-5.29 (m, 1H), 5.05-4.89 (m, 1H), |
| | | | C₂₇H₃₉N₂O₁₁, | 4.80 (td, J = 8.9, 2.5 Hz, 1H), 4.02-3.85 (m, 5H), |
| | | | 567.2548; | 3.75 (t, J = 11.9 Hz, 1H), 3.68-3.54 (m, 1H), |
| | | | found, | 3.54-3.29 (m, 3H), 2.14 (t, J = 13.6 Hz, 1H), |
| | | | 567.2555 | 2.07 (d, J = 2.8 Hz, 3H), 1.71-1.54 (m, 2H), |
| | | | | 1.46 (d, J = 6.7 Hz, 2H), 1.40-1.22 (m, 5H), |
| | | | | 1.12-1.00 (m, 2H), 0.96-0.83 (m, 5H) |
| | | | | ¹³C NMR (101 MHz, CDCl₃) δ 174.04, |
| | | | | 170.22, 169.35, 163.36, 160.25, 145.84, |
| | | | | 143.98, 142.27, 109.69, 89.48, 76.14, 75.76, |
| | | | | 72.92, 72.01, 67.59, 67.40, 56.19, 53.95, |
| | | | | 33.73, 32.21, 20.85, 19.23, 18.69, 13.94, |
| | | | | 12.79, 8.77, 8.52 |
| 6 | — | — | HRMS-ESI | ¹H NMR (400 MHz, CDCl₃) δ 8.73 (d, J = 7.9 Hz, |
| | | | (m/z) | 1H), 8.30 (d, J = 5.4 Hz, 1H), 6.96 (d, J = 5.4 Hz, |
| | | | [M + H]⁺ | 1H), 5.84-5.65 (m, 2H), 5.19 (dq, J = 8.9, |
| | | | calcd for | 6.3 Hz, 1H), 4.93 (dt, J = 8.0, 2.3 Hz, |
| | | | C₂₇H₄₃N₂O₁₀, | 1H), 3.91 (s, 5H), 3.80 (dd, J = 8.7, 5.9 Hz, |
| | | | 555.2912; | 1H), 3.73 (td, J = 12.3, 1.6 Hz, 1H), 3.64 (dd, |
| | | | found, | J = 9.2, 6.4 Hz, 1H), 3.42 (dt, J = 12.1, 2.8 Hz, |
| | | | 555.2914 | 1H), 3.33 (t, J = 7.4 Hz, 1H), 3.13 (ddd, J = 15.6, |
| | | | | 9.0, 7.1 Hz, 2H), 3.03 (t, J = 8.9 Hz, |
| | | | | 1H), 2.07 (m, 4H), 1.81 (ddt, J = 22.8, 13.3, |
| | | | | 6.7 Hz, 2H), 1.50-1.39 (m, 1H), 1.37 (d, J = 6.3 Hz, |
| | | | | 3H), 0.98-0.81 (m, 12H) |
| | | | | ¹³C NMR (101 MHz, CDCl₃) δ 170.32, |
| | | | | 169.35, 163.47, 160.31, 145.96, 143.99, |
| | | | | 142.51, 109.72, 89.61, 85.69, 80.59, 80.15, |
| | | | | 78.61, 73.49, 67.30, 56.27, 54.13, 34.40, |
| | | | | 29.17, 29.07, 20.95, 19.71, 19.54, 19.48, |
| | | | | 19.43, 19.11 |
| 7 | — | — | HRMS-ESI | ¹H NMR (400 MHz, CDCl₃) δ 8.73 (d, J = 7.9 Hz, |
| | | | (m/z) | 1H), 8.31 (d, J = 5.4 Hz, 1H), 6.96 (d, J = 5.4 Hz, |
| | | | [M + H]⁺ | 1H), 5.85-5.66 (m, 2H), 5.19 (dq, J = 8.8, |
| | | | calcd for | 6.3 Hz, 1H), 4.93 (dt, J = 8.0, 2.3 Hz, |
| | | | C₂₇H₄₃N₂O₁₀, | 1H), 3.91 (d, J = 1.9 Hz, 5H), 3.87-3.65 (m, |
| | | | 555.2912; | 3H), 3.42 (ddd, J = 13.9, 6.8, 2.5 Hz, 2H), |
| | | | found, | 3.33 (t, J = 7.4 Hz, 1H), 3.13 (dd, J = 8.7, 7.1 Hz, |
| | | | 555.2908 | 1H), 3.03 (t, J = 8.9 Hz, 1H), 2.07 (s, 4H), |
| | | | | 1.84 (dt, J = 13.2, 6.7 Hz, 1H), |
| | | | | 1.61-1.41 (m, 3H), 1.41-1.27 (m, 5H), 1.00-0.82 (m, |
| | | | | 9H) |
| | | | | ¹³C NMR (101 MHz, CDCl₃) δ 170.23, |
| | | | | 169.26, 163.37, 160.21, 145.86, 143.90, |
| | | | | 142.41, 109.62, 89.51, 85.56, 80.54, 78.07, |
| | | | | 73.43, 72.89, 67.20, 67.14, 56.17, 54.04, |
| | | | | 34.28, 32.47, 29.09, 20.85, 19.58, 19.39, |
| | | | | 19.27, 19.03, 13.99 |
| 8 | — | — | HRMS-ESI | ¹H NMR (400 MHz, CDCl₃) δ 8.52 (d, J = 7.3 Hz, |
| | | | (m/z) | 1H), 8.29 (d, J = 5.4 Hz, 1H), |
| | | | [M + H]⁺ | 7.45-7.21 (m, 10H), 6.95 (d, J = 5.4 Hz, 1H), 5.77 (d, J = 6.4 Hz, |
| | | | calcd for | 1H), 5.73 (d, J = 6.4 Hz, 1H), |
| | | | C₃₄H₄₁N₂O₉, | 5.09-4.99 (m, 1H), 4.98 (d, J = 10.7 Hz, 1H), |
| | | | 621.2807; | 4.76 (ddd, J = 7.4, 5.3, 3.5 Hz, 1H), |
| | | | found, | 4.68-4.56 (m, 3H), 3.90 (s, 3H), 3.48 (t, J = 8.8 Hz, 1H), |
| | | | 621.2844 | 3.39 (ddd, J = 8.4, 5.5, 3.0 Hz, 1H), |
| | | | | 2.16-2.08 (m, 1H), 2.07 (s, 3H), 2.06-1.99 (m, |
| | | | | 1H), 1.87 (td, J = 14.2, 13.0, 6.0 Hz, 1H), |
| | | | | 1.65-1.42 (m, 4H), 1.40 (d, J = 6.3 Hz, 3H), |
| | | | | 1.30-1.16 (m, 1H) |
| | | | | ¹³C NMR (101 MHz, CDCl₃) δ 172.40, |
| | | | | 170.27, 163.01, 160.28, 145.80, 143.94, |
| | | | | 142.54, 138.62, 138.29, 128.38, 128.34, |
| | | | | 128.04, 127.95, 127.70, 127.55, 109.57, |
| | | | | 89.57, 84.07, 82.52, 75.77, 72.64, 72.62, |
| | | | | 56.19, 51.90, 28.65, 27.17, 22.41, 21.69, |
| | | | | 20.88, 18.34 |
| 9 | — | — | HRMS-ESI | ¹H NMR (400 MHz, CDCl₃) δ 8.81-8.62 (m, |
| | | | (m/z) | 1H), 8.34 (d, J = 5.4 Hz, 1H), 7.41-7.21 (m, |
| | | | [M + H]⁺ | 10H), 6.99 (d, J = 5.5 Hz, 1H), 5.02 (dd, J = 6.3, |
| | | | calcd for | 2.8 Hz, 1H), 4.97 (d, J = 11.0 Hz, 1H), |
| | | | C₃₃H₃₉N₂O₈, | 4.73 (ddd, J = 7.8, 5.2, 3.6 Hz, 1H), |
| | | | 591.2701; | 4.67-4.61 (m, 2H), 4.59 (d, J = 11.5 Hz, 1H), |
| | | | found, | 3.89 (s, 3H), 3.46 (t, J = 8.8 Hz, 1H), 3.38 (ddd, J = 8.3, |
| | | | 591.2704 | 5.4, 2.9 Hz, 1H), 2.39 (s, 3H), |
| | | | | 2.11 (ddt, J = 11.3, 7.6, 3.7 Hz, 1H), |
| | | | | 2.07-1.97 (m, 1H), 1.91-1.77 (m, 1H), 1.64-1.45 (m, |
| | | | | 3H), 1.45-1.40 (m, 1H), 1.38 (d, J = 6.3 Hz, |
| | | | | 3H), 1.24-1.15 (m, 1H) |
| | | | | ¹³C NMR (101 MHz, CDCl₃) δ 172.28, |
| | | | | 168.91, 162.46, 159.43, 146.77, 141.54, |
| | | | | 138.62, 138.30, 137.45, 128.38, 128.34, |
| | | | | 128.04, 127.96, 127.71, 127.56, 109.79, |
| | | | | 84.08, 82.48, 75.75, 72.66, 72.63, 56.29, |
| | | | | 51.70, 28.70, 27.22, 22.44, 21.61, 20.77, |
| | | | | 18.34, 14.22 |
| 10 | — | — | HRMS-ESI | ¹H NMR (400 MHz, CDCl₃) δ 8.55 (d, J = 7.3 Hz, |
| | | | (m/z) | 1H), 8.34-8.20 (m, 1H), 7.33-7.18 (m, |
| | | | [M + H]⁺ | 2H), 7.01-6.84 (m, 4H), 5.79-5.75 (m, |
| | | | calcd for | 1H), 5.75-5.71 (m, 1H), 5.02 (dt, J = 12.6, |
| | | | C₃₀H₄₁N₂O₉, | 6.3 Hz, 1H), 4.79 (t, J = 7.6 Hz, 1H), 4.15 (t, J = 6.5 Hz, |
| | | | 573.2807; | 1H), 3.96-3.87 (m, 3H), |
| | | | found, | 3.67-3.59 (m, 1H), 3.43 (t, J = 8.8 Hz, 1H), |
| | | | 573.2821 | 3.38-3.25 (m, 1H), 2.22 (d, J = 3.8 Hz, 1H), |
| | | | | 2.12-2.00 (m, 4H), 2.00-1.89 (m, 1H), 1.68 (td, J = 14.9, |
| | | | | 13.0, 8.3 Hz, 2H), 1.60-1.48 (m, |
| | | | | 3H), 1.44 (d, J = 6.2 Hz, 3H), 1.29-1.15 (m, |
| | | | | 1H), 0.79 (d, J = 6.6 Hz, 3H), 0.75 (d, J = 6.6 Hz, |
| | | | | 3H) |
| | | | | ¹³C NMR (101 MHz, CDCl₃) δ 172.52, |
| | | | | 170.27, 163.04, 160.27, 158.04, 145.82, |
| | | | | 143.92, 142.52, 129.36, 120.59, 115.70, |
| | | | | 109.59, 89.55, 83.75, 80.89, 80.71, 72.44, |
| | | | | 56.19, 51.59, 28.94, 28.07, 26.97, 21.81, |
| | | | | 21.72, 20.88, 19.39, 19.31, 18.21 |
| 11 | — | — | HRMS-ESI | ¹H NMR (400 MHz, CDCl₃) δ 8.55 (d, J = 7.3 Hz, |
| | | | (m/z) | 1H), 8.30 (d, J = 5.4 Hz, 1H), |
| | | | [M + H]⁺ | 7.33-7.21 (m, 2H), 7.07-7.00 (m, 2H), 7.00-6.92 (m, |
| | | | calcd for | 2H), 5.77 (d, J = 6.4 Hz, 1H), 5.74 (d, J = 6.4 Hz, |
| | | | C₂₇H₃₅N₂O₉, | 1H), 5.14 (dq, J = 9.4, 6.3 Hz, 1H), |
| | | | 531.2337; | 4.79 (ddd, J = 7.4, 5.4, 3.5 Hz, 1H), 4.26 (t, J = 8.9 Hz, |
| | | | found, | 1H), 3.92 (s, 3H), 3.31 (s, 3H), 3.22 (tt, J = 5.9, |
| | | | 531.2340 | 3.1 Hz, 1H), 2.26-2.14 (m, 1H), |
| | | | | 2.14-2.08 (m, 1H), 2.07 (s, 3H), 1.97-1.83 (m, |
| | | | | 1H), 1.69 (dd, J = 12.7, 4.5 Hz, 2H), 1.52 (dt, |
| | | | | J = 8.7, 4.7 Hz, 2H), 1.34 (d, J = 6.3 Hz, 3H), |
| | | | | 1.33-1.24 (m, 1H) |
| | | | | ¹³C NMR (101 MHz, CDCl₃) δ 172.45, |
| | | | | 170.31, 163.03, 160.28, 159.61, 145.83, |
| | | | | 143.95, 142.49, 129.36, 121.24, 116.17, |
| | | | | 109.61, 89.55, 83.68, 82.97, 72.37, 58.54, |
| | | | | 56.20, 51.88, 28.03, 22.32, 21.84, 20.88, |
| | | | | 18.36 |
| 12 | — | — | HRMS-ESI | ¹H NMR (400 MHz, CDCl₃) δ 8.53 (d, J = 7.3 Hz, |
| | | | (m/z) | 1H), 8.29 (d, J = 5.4 Hz, 1H), 6.96 (d, J = 5.4 Hz, |
| | | | [M + H]⁺ | 1H), 5.76 (d, J = 6.4 Hz, 1H), 5.73 (d, |
| | | | calcd for | J = 6.4 Hz, 1H), 4.91 (dq, J = 9.3, 6.3 Hz, |
| | | | C₂₅H₃₉N₂O₉, | 1H), 4.74 (ddd, J = 7.4, 5.6, 3.4 Hz, 1H), |
| | | | 511.2650; | 3.91 (s, 3H), 3.64 (dd, J = 8.5, 6.5 Hz, 1H), 3.40 (s, |
| | | | found, | 3H), 3.31 (dd, J = 8.5, 6.5 Hz, 1H), 3.17 (t, J = 8.8 Hz, |
| | | | 511.2663 | 1H), 3.03 (ddd, J = 8.5, 5.9, 2.9 Hz, |
| | | | | 1H), 2.13 (ddt, J = 12.4, 8.8, 4.4 Hz, 1H), |
| | | | | 2.07 (s, 3H), 2.06-1.96 (m, 1H), 1.92-1.72 (m, |
| | | | | 2H), 1.61 (dtd, J = 15.6, 8.0, 7.6, 3.8 Hz, 2H), |
| | | | | 1.55-1.44 (m, 1H), 1.38 (d, J = 6.3 Hz, 3H), |
| | | | | 1.36-1.30 (m, 1H), 1.24 (qt, J = 13.4, 6.8 Hz, |
| | | | | 1H), 0.92 (d, J = 6.7 Hz, 6H) |
| | | | | ¹³C NMR (101 MHz, CDCl₃) δ 172.46, |
| | | | | 170.24, 162.97, 160.24, 145.78, 143.88, |
| | | | | 142.51, 109.56, 89.51, 84.87, 84.28, 80.66, |
| | | | | 72.71, 58.05, 56.17, 51.83, 29.09, 27.88, |
| | | | | 27.33, 22.23, 21.92, 20.85, 19.49, 19.46, |
| | | | | 18.16 |
| 13 | — | — | HRMS-ESI | ¹H NMR (400 MHz, CDCl₃) δ 8.55 (d, J = 7.3 Hz, |
| | | | (m/z) | 1H), 8.30 (d, J = 5.4 Hz, 1H), |
| | | | [M + H]⁺ | 7.34-7.18 (m, 2H), 7.11-6.99 (m, 2H), 6.99-6.86 (m, |
| | | | calcd for | 2H), 5.77 (d, J = 6.4 Hz, 1H), 5.74 (d, J = 6.4 Hz, |
| | | | C₃₀H₄₁N₂O₉, | 1H), 5.13 (dq, J = 9.3, 6.3 Hz, 1H), |
| | | | 573.2807; | 4.79 (ddd, J = 7.4, 5.5, 3.4 Hz, 1H), 4.29 (t, J = 8.9 Hz, |
| | | | found, | 1H), 3.91 (s, 3H), 3.29 (ddd, J = 8.6, 6.0, |
| | | | 573.2827 | 2.9 Hz, 1H), 3.22 (dd, J = 8.9, 6.4 Hz, 1H), |
| | | | | 3.16 (dd, J = 8.9, 6.5 Hz, 1H), 2.30-2.17 (m, |
| | | | | 1H), 2.11 (dd, J = 8.6, 3.6 Hz, 1H), 2.07 (s, |
| | | | | 3H), 1.91 (dt, J = 12.6, 4.9 Hz, 1H), |
| | | | | 1.78-1.61 (m, 2H), 1.56 (tt, J = 14.4, 7.3 Hz, 3H), |
| | | | | 1.34 (d, J = 6.3 Hz, 3H), 1.32-1.22 (m, 1H), |
| | | | | 0.72 (d, J = 6.7 Hz, 3H), 0.66 (d, J = 6.7 Hz, |
| | | | | 3H) |
| | | | | ¹³C NMR (101 MHz, CDCl₃) δ 172.47, |
| | | | | 170.26, 163.01, 160.27, 159.56, 145.80, |
| | | | | 143.93, 142.49, 129.17, 120.97, 116.04, |
| | | | | 109.61, 89.53, 82.49, 82.15, 77.71, 72.40, |
| | | | | 56.19, 51.82, 28.72, 28.21, 27.29, 22.34, |
| | | | | 21.90, 20.87, 19.28, 19.17, 18.34 |
| 14 | — | — | HRMS-ESI | ¹H NMR (400 MHz, CDCl₃) δ 8.52 (d, J = 7.3 Hz, |
| | | | (m/z) | 1H), 8.29 (d, J = 5.4 Hz, 1H), 6.96 (d, J = 5.4 Hz, |
| | | | [M + H]⁺ | 1H), 5.76 (d, J = 6.4 Hz, 1H), 5.73 (d, |
| | | | calcd for | J = 6.4 Hz, 1H), 4.90 (dq, J = 9.1, 6.3 Hz, |
| | | | C₂₈H₄₅N₂O₉, | 1H), 4.74 (ddd, J = 7.4, 5.7, 3.4 Hz, 1H), |
| | | | 553.3120; | 3.91 (s, 3H), 3.72 (dd, J = 8.4, 6.0 Hz, 1H), |
| | | | found, | 3.36-3.20 (m, 3H), 3.18 (d, J = 8.9 Hz, 1H), |
| | | | 553.3136 | 3.15-3.06 (m, 1H), 2.14 (dd, J = 7.5, 3.7 Hz, 1H), |
| | | | | 2.07 (s, 3H), 2.06-1.97 (m, 1H), 1.81 (tq, J = 13.2, |
| | | | | 6.7 Hz, 3H), 1.61 (ddt, J = 11.1, 8.5, 4.0 Hz, |
| | | | | 2H), 1.56-1.43 (m, 1H), 1.38 (d, J = 6.3 Hz, |
| | | | | 3H), 1.35 (d, J = 3.1 Hz, 1H), 1.23 (td, J = 8.9, |
| | | | | 4.2 Hz, 1H), 0.95-0.87 (m, 12H) |
| | | | | ¹³C NMR (101 MHz, CDCl₃) δ 172.50, |
| | | | | 170.25, 162.97, 160.25, 145.78, 143.88, |
| | | | | 142.55, 109.55, 89.54, 84.12, 83.27, 80.59, |
| | | | | 72.77, 56.17, 51.79, 29.10, 28.94, 28.15, |
| | | | | 27.34, 22.29, 22.02, 20.85, 19.61, 19.55, |
| | | | | 19.50, 19.38, 18.15 |
| 15 | — | — | HRMS-ESI | ¹H NMR (400 MHz, CDCl₃) δ 8.58 (d, J = 7.4 Hz, |
| | | | (m/z) | 1H), 8.30 (dd, J = 5.4, 1.1 Hz, 1H), |
| | | | [M + H]⁺ | 8.14-7.99 (m, 2H), 7.62-7.52 (m, 1H), |
| | | | calcd for | 7.50-7.38 (m, 2H), 6.96 (d, J = 5.5 Hz, 1H), 5.77 (d, J = 6.4 Hz, |
| | | | C₃₂H₄₃N₂O₁₀, | 1H), 5.75 (d, J = 6.4 Hz, 1H), 5.06 (dt, |
| | | | 615.2912; | J = 9.3, 6.3 Hz, 2H), 4.81 (ddd, J = 7.5, 5.3, |
| | | | found, | 3.6 Hz, 1H), 3.92 (s, 3H), 3.70-3.52 (m, |
| | | | 615.2926 | 2H), 3.45 (t, J = 9.0 Hz, 1H), 2.17 (s, 1H), |
| | | | | 2.13-1.96 (m, 5H), 1.83-1.75 (m, 1H), |
| | | | | 1.60 (dt, J = 18.9, 6.7 Hz, 3H), 1.43 (d, J = 6.3 Hz, |
| | | | | 3H), 1.41-1.19 (m, 3H), 1.15-1.03 (m, |
| | | | | 3H), 0.88-0.54 (m, 4H) |
| | | | | ¹³C NMR (101 MHz, CDCl₃) δ 172.24, |
| | | | | 170.28, 165.68, 163.07, 160.26, 145.85, |
| | | | | 143.87, 142.48, 132.88, 130.48, 129.53, |
| | | | | 128.36, 109.60, 89.53, 82.98, 74.06, 72.69, |
| | | | | 56.19, 51.71, 29.78, 28.94, 28.12, 26.97, |
| | | | | 22.37, 22.01, 21.62, 20.88, 18.05, 13.83 |
| 16 | — | — | ESIMS | ¹H NMR (400 MHz, CDCl₃) δ 8.70 (d, J = 6.4 Hz, |
| | | | m/z 572 | 1H), 8.27 (d, J = 4.9 Hz, 1H), 7.29 (m, |
| | | | ([M + H]⁺) | 2H), 7.23-7.16 (m, 3H), 6.94 (d, J = 5.3 Hz, |
| | | | | 1H), 5.79-5.69 (m, 2H), 5.10 (dq, J = 9.4, |
| | | | | 6.4 Hz, 1H), 4.71-4.65 (m, 1H), 3.90 (s, |
| | | | | 3H), 3.72 (t, J = 11.1 Hz, 1H), 3.45 (dd, J = 8.2, |
| | | | | 6.5 Hz, 1H), 3.39-3.22 (m, 4H), |
| | | | | 3.18 (dt, J = 12.4, 3.3 Hz, 1H), 3.07 (t, J = 9.3 Hz, |
| | | | | 1H), 2.54 (td, J = 11.5, 3.6 Hz, 1H), |
| | | | | 2.17-2.03 (m, 2H), 2.06 (s, 3H), 1.89 (dt, J = 13.1, |
| | | | | 6.5 Hz, 2H), 1.42 (d, J = 6.4 Hz, 3H), |
| | | | | 0.95 (dd, J = 6.7, 1.1 Hz, 6H) |
| | | | | ¹³C NMR (101 MHz, CDCl₃) δ 172.04, |
| | | | | 170.24, 163.16, 160.15, 145.90, 143.68, |
| | | | | 142.64, 140.04, 129.14, 128.42, 126.04, |
| | | | | 109.55, 89.49, 83.87, 78.74, 74.62, 68.62, |
| | | | | 65.14, 56.17, 50.19, 46.61, 35.12, 29.14, |
| | | | | 28.91, 20.87, 19.55, 19.52, 18.54 |
| 17 | — | — | HRMS-ESI | ¹H NMR (400 MHz, CDCl₃) δ 8.57 (d, J = 7.2 Hz, |
| | | | (m/z) | 1H), 8.29 (d, J = 5.4 Hz, 1H), |
| | | | [M + H]⁺ | 7.34-7.18 (m, 2H), 7.02-6.82 (m, 4H), 5.77 (d, J = 6.4 Hz, |
| | | | calcd for | 1H), 5.74 (d, J = 6.4 Hz, 1H), 5.02 (dq, J = 9.5, |
| | | | C₃₁H₄₃N₂O₉, | 6.2 Hz, 1H), 4.88-4.72 (m, 1H), |
| | | | 587.2963; | 4.22-4.09 (m, 1H), 3.90 (s, 3H), 3.83 (dt, J = 8.6, |
| | | | found, | 6.6 Hz, 1H), 3.57 (dt, J = 8.7, 6.7 Hz, 1H), |
| | | | 587.2994 | 3.51-3.38 (m, 1H), 2.33-2.12 (m, 1H), |
| | | | | 2.12-2.01 (m, 4H), 2.01-1.87 (m, 1H), |
| | | | | 1.78-1.63 (m, 1H), 1.53 (tt, J = 17.8, 9.5 Hz, 3H), |
| | | | | 1.46-1.34 (m, 5H), 1.31-1.11 (m, 5H), |
| | | | | 0.88-0.66 (m, 3H) |
| | | | | ¹³C NMR (101 MHz, CDCl₃) δ 172.51, |
| | | | | 170.23, 163.04, 160.27, 158.12, 145.82, |
| | | | | 143.91, 142.48, 129.37, 120.62, 115.70, |
| | | | | 115.54, 109.63, 89.50, 83.84, 80.91, 74.04, |
| | | | | 72.37, 56.20, 51.57, 29.86, 28.21, 28.12, |
| | | | | 22.45, 21.84, 21.70, 20.87, 18.16, 13.95 |
| 18 | — | — | HRMS-ESI | ¹H NMR (400 MHz, CDCl₃) δ 8.56 (d, J = 7.2 Hz, |
| | | | (m/z) | 1H), 8.38-8.18 (m, 1H), 7.29-7.22 (m, |
| | | | [M + H]⁺ | 2H), 7.06-6.86 (m, 4H), 5.77 (dd, J = 6.4, |
| | | | calcd for | 1.1 Hz, 1H), 5.74 (dd, J = 6.4, 1.0 Hz, 1H), |
| | | | C₂₉H₃₉N₂O₉, | 5.01 (dt, J = 12.6, 6.3 Hz, 1H), |
| | | | 559.2650; | 4.85-4.69 (m, 1H), 4.14 (t, J = 7.2 Hz, 1H), |
| | | | found, | 3.99-3.87 (m, 3H), 3.80 (q, J = 6.8 Hz, 1H), 3.54 (q, J = 7.8, |
| | | | 559.2683 | 7.3 Hz, 1H), 3.45 (t, J = 8.9 Hz, 1H), |
| | | | | 2.23 (s, 1H), 2.12-2.01 (m, 4H), 2.01-1.88 (m, |
| | | | | 1H), 1.77-1.63 (m, 1H), 1.63-1.50 (m, |
| | | | | 3H), 1.50-1.38 (m, 4H), 1.38-1.30 (m, |
| | | | | 1H), 1.30-1.15 (m, 1H), 0.83-0.74 (m, 3H) |
| | | | | ¹³C NMR (101 MHz, CDCl₃) δ 172.51, |
| | | | | 170.24, 163.03, 160.27, 158.12, 145.82, |
| | | | | 143.90, 142.48, 129.37, 120.62, 115.71, |
| | | | | 109.62, 89.50, 83.81, 80.98, 75.62, 72.37, |
| | | | | 56.19, 51.55, 28.09, 26.91, 23.33, 21.82, |
| | | | | 21.70, 20.85, 18.15, 10.54 |
| 19 | — | — | HRMS-ESI | ¹H NMR (400 MHz, CDCl₃) δ 8.59 (d, J = 7.3 Hz, |
| | | | (m/z) | 1H), 8.30 (d, J = 5.4 Hz, 1H), |
| | | | [M + H]⁺ | 7.27-7.21 (m, 2H), 7.21-7.15 (m, 2H), 7.02-6.85 (m, |
| | | | calcd for | 5H), 6.71 (dd, J = 8.7, 0.9 Hz, 2H), 5.77 (d, J = 6.4 Hz, |
| | | | C₃₂H₃₇N₂O₉, | 1H), 5.74 (d, J = 6.4 Hz, 1H), |
| | | | 593.2494; | 5.27-5.19 (m, 1H), 4.84 (ddd, J = 7.4, 5.2, 3.5 Hz, |
| | | | found, | 1H), 4.53 (t, J = 8.9 Hz, 1H), 4.36-4.22 (m, |
| | | | 593.2511 | 1H), 3.90 (s, 3H), 2.33-2.20 (m, 1H), |
| | | | | 2.15-2.02 (m, 5H), 1.79 (ddd, J = 14.3, 11.3, 5.4 Hz, |
| | | | | 1H), 1.74-1.64 (m, 1H), 1.64-1.53 (m, |
| | | | | 2H), 1.41 (d, J = 6.3 Hz, 3H), 1.35-1.21 (m, |
| | | | | 1H) |
| | | | | ¹³C NMR (101 MHz, CDCl₃) δ 172.50, |
| | | | | 170.28, 163.09, 160.31, 159.45, 157.86, |
| | | | | 145.85, 143.98, 142.46, 129.33, 129.25, |
| | | | | 121.35, 120.86, 116.30, 115.74, 109.67, |
| | | | | 89.54, 82.17, 80.32, 72.11, 56.22, 51.53, |
| | | | | 27.98, 26.90, 21.85, 21.71, 20.90, 18.43 |
| 20 | — | (Neat) | HRMS-ESI | ¹H NMR (400 MHz, CDCl₃) δ 8.66 (d, J = 8.0 Hz, |
| | | 3385, | (m/z) | 1H), 8.30 (d, J = 5.4 Hz, 1H), |
| | | 2956, | [M]⁺ | 7.32-7.09 (m, 5H), 6.94 (d, J = 5.4 Hz, 1H), |
| | | 2872, | calcd for | 5.78-5.67 (m, 2H), 5.19 (dq, J = 9.1, 6.3 Hz, 1H), |
| | | 1745, | C₃₀H₄₀N₂O₉, | 4.91 (ddd, J = 7.9, 3.4, 1.6 Hz, 1H), 3.90 (s, 3H), |
| | | 1676, | 572.2734; | 3.86-3.73 (m, 2H), 3.46 (dd, J = 8.4, 6.2 Hz, |
| | | 1504, | found, | 1H), 3.36-3.23 (m, 2H), 3.07-2.93 (m, |
| | | 1201, | 572.2773 | 2H), 2.68-2.56 (m, 1H), 2.21 (dd, J = 13.1, |
| | | 1085 | | 11.4 Hz, 1H), 2.14-1.97 (m, 4H), |
| | | | | 1.95-1.80 (m, 2H), 1.39 (d, J = 6.3 Hz, 3H), |
| | | | | 1.39-1.21 (m, 1H), 0.94 (dd, J = 6.7, 3.9 Hz, 6H) |
| | | | | ¹³C NMR (101 MHz, CDCl₃) δ 170.23, |
| | | | | 169.37, 163.38, 160.18, 145.86, 143.83, |
| | | | | 142.49, 140.85, 129.24, 128.36, 125.86, |
| | | | | 109.58, 89.53, 85.70, 80.67, 74.62, 68.86, |
| | | | | 68.35, 56.17, 53.97, 39.41, 38.64, 32.49, |
| | | | | 29.14, 20.86, 19.57, 19.52, 19.07 |
| 21 | — | (Neat) | HRMS-ESI | ¹H NMR (400 MHz, CDCl₃) δ 8.81 (d, J = 8.2 Hz, |
| | | 3386, | (m/z) | 1H), 8.35 (d, J = 5.5 Hz, 1H), |
| | | 2956, | [M]⁺ | 7.30-7.10 (m, 5H), 7.03-6.93 (m, 1H), 5.19 (dq, J = 9.3, |
| | | 2872, | calcd for | 6.3 Hz, 1H), 4.88 (ddd, J = 8.2, 3.6, 1.4 Hz, |
| | | 1770, | C₂₉H₃₈N₂O₈, | 1H), 3.88 (s, 3H), 3.83-3.67 (m, 2H), |
| | | 1743, | 542.2628; | 3.46 (dd, J = 8.4, 6.2 Hz, 1H), 3.36-3.22 (m, |
| | | 1678, | found, | 2H), 3.06-2.91 (m, 2H), 2.68-2.56 (m, |
| | | 1507, | 546.2650 | 1H), 2.37 (s, 3H), 2.28-2.14 (m, 1H), |
| | | 1085 | | 2.12-1.99 (m, 1H), 1.94-1.79 (m, 2H), 1.38 (d, J = 6.3 Hz, |
| | | | | 3H), 1.36-1.23 (m, 1H), 0.93 (dd, |
| | | | | J = 6.7, 3.7 Hz, 6H) |
| | | | | ¹³C NMR (101 MHz, CDCl₃) δ 169.24, |
| | | | | 168.80, 162.85, 159.32, 146.83, 141.42, |
| | | | | 140.84, 137.44, 129.25, 128.37, 125.87, |
| | | | | 109.78, 85.70, 80.66, 74.61, 68.87, 68.42, |
| | | | | 56.27, 53.76, 39.41, 38.65, 32.48, 29.14, |
| | | | | 20.72, 19.57, 19.52, 19.05 |
| 22 | — | (Neat) | HRMS-ESI | ¹H NMR (400 MHz, CDCl₃) δ 8.51 (d, J = 7.3 Hz, |
| | | 3335, | (m/z) | 1H), 8.28 (d, J = 5.4 Hz, 1H), 6.95 (d, J = 5.4 Hz, |
| | | 2953, | [M + H]⁺ | 1H), 5.76 (d, J = 6.4 Hz, 1H), 5.73 (d, |
| | | 1752, | calcd for | J = 6.4 Hz, 1H), 4.89 (dq, J = 8.9, 6.3 Hz, |
| | | 1740, | C₂₂H₃₃N₂O₉, | 1H), 4.75 (ddd, J = 7.1, 5.5, 3.4 Hz, 1H), |
| | | 1724, | 469.2181; | 3.91 (s, 3H), 3.57 (s, 3H), 3.42 (s, 3H), 3.11 (t, J = 8.7 Hz, |
| | | 1682, | found, | 1H), 3.03 (ddd, J = 8.5, 5.9, 2.9 Hz, |
| | | 1504, | 469.2201 | 1H), 2.21-1.99 (m, 2H), 2.07 (s, 3H), |
| | | 1206 | | 1.79 (ddt, J = 14.4, 8.7, 5.8 Hz, 1H), |
| | | | | 1.69-1.45 (m, 3H), 1.44-1.34 (m, 1H), 1.38 (d, J = 6.3 Hz, |
| | | | | 3H), 1.30-1.19 (m, 1H) |
| 23 | — | (Neat) | HRMS-ESI | ¹H NMR (400 MHz, CDCl₃) δ 8.53 (d, J = 7.3 Hz, |
| | | 3378, | (m/z) | 1H), 8.30 (d, J = 5.4 Hz, 1H), |
| | | 2938, | [M + H]⁺ | 7.29-7.22 (m, 2H), 7.05-7.00 (m, 2H), 6.97-6.91 (m, |
| | | 1737, | calcd for | 2H), 5.77 (d, J = 6.4 Hz, 1H), 5.74 (d, J = 6.4 Hz, |
| | | 1676, | C₂₉H₃₉N₂O₉, | 1H), 5.14 (dq, J = 9.3, 6.3 Hz, 1H), |
| | | 1492, | 559.2650; | 4.79 (ddd, J = 7.4, 5.4, 3.5 Hz, 1H), 4.27 (dd, J = 9.3, |
| | | 1195 | found, | 8.4 Hz, 1H), 3.91 (s, 3H), 3.44-3.26 (m, |
| | | | 559.2678 | 3H), 2.24-2.15 (m, 1H), 2.15-2.08 (m, |
| | | | | 1H), 2.07 (s, 3H), 1.98-1.85 (m, 1H), |
| | | | | 1.75-1.59 (m, 2H), 1.60-1.46 (m, 2H), |
| | | | | 1.39-1.28 (m, 3H), 1.34 (d, J = 6.4 Hz, 3H), 0.70 (t, J = 7.4 Hz, |
| | | | | 3H) |
| 24 | — | — | HRMS-ESI | ¹H NMR (400 MHz, CDCl₃) δ 8.50 (d, J = 7.4 Hz, |
| | | | (m/z) | 1H), 8.27 (d, J = 5.4 Hz, 1H), |
| | | | [M + H]⁺ | 7.38-7.22 (m, 2H), 7.18 (d, J = 7.1 Hz, 3H), 6.94 (d, J = 5.4 Hz, |
| | | | calcd for | 1H), 5.88-5.63 (m, 2H), 5.00 (dq, J = 9.2, |
| | | | C₃₁H₄₃N₂O₈, | 6.3 Hz, 1H), 4.87-4.64 (m, 1H), |
| | | | 571.3014; | 3.89 (s, 3H), 3.47-3.33 (m, 2H), 3.27 (dd, J = 13.2, |
| | | | found, | 3.1 Hz, 1H), 3.07 (t, J = 9.3 Hz, 1H), |
| | | | 571.3025 | 2.26-2.18 (m, 1H), 2.12 (dq, J = 8.0, 4.6 Hz, |
| | | | | 1H), 2.06 (s, 3H), 2.03-1.93 (m, 1H), |
| | | | | 1.87 (dq, J = 13.2, 6.6 Hz, 1H), 1.81-1.68 (m, |
| | | | | 1H), 1.42 (d, J = 6.3 Hz, 4H), 1.38 (d, J = 11.1 Hz, |
| | | | | 2H), 1.32-1.08 (m, 2H), |
| | | | | 0.98-0.93 (m, 6H), 0.93-0.84 (m, 1H) |
| | | | | ¹³C NMR (101 MHz, CDCl₃) δ 172.61, |
| | | | | 170.23, 163.00, 160.25, 145.80, 143.87, |
| | | | | 142.59, 141.05, 129.12, 128.25, 125.74, |
| | | | | 109.55, 89.57, 84.58, 79.69, 75.50, 56.18, |
| | | | | 51.66, 45.65, 38.26, 30.92, 29.16, 27.15, |
| | | | | 26.00, 24.38, 22.07, 20.87, 19.57, 19.53, |
| | | | | 18.46 |
| 25 | — | (Neat) | HRMS-ESI | ¹H NMR (400 MHz, CDCl₃) δ 8.76 (d, J = 7.4 Hz, |
| | | 3375, | (m/z) | 1H), 8.36 (d, J = 5.4 Hz, 1H), |
| | | 2942, | [M + H]⁺ | 7.25-7.15 (m, 4H), 7.03-6.85 (m, 5H), 6.73-6.68 (m, |
| | | 1770, | calcd for | 2H), 5.24 (dq, J = 9.4, 6.3 Hz, 1H), 4.82 (ddd, |
| | | 1735, | C₃₁H₃₅N₂O₈, | J = 7.6, 5.1, 3.5 Hz, 1H), 4.51 (dd, J = 9.4, 8.3 Hz, |
| | | 1677, | 563.2388; | 1H), 4.28 (ddd, J = 8.7, 7.1, 2.4 Hz, 1H), |
| | | 1491, | found, | 3.91 (s, 3H), 2.40 (s, 3H), 2.30-2.19 (m, |
| | | 1192 | 563.2406 | 1H), 2.13-2.01 (m, 2H), 1.84-1.53 (m, |
| | | | | 4H), 1.40 (d, J = 6.3 Hz, 3H), 1.36-1.23 (m, |
| | | | | 1H) |
| 26 | — | (Neat) | HRMS-ESI | ¹H NMR (400 MHz, CDCl₃) δ 8.73 (d, J = 7.5 Hz, |
| | | 3379, | (m/z) | 1H), 8.36 (d, J = 5.4 Hz, 1H), |
| | | 2938, | [M + H]⁺ | 7.29-7.23 (m, 2H), 7.05-6.99 (m, 3H), 6.93 (tt, J = 7.3, |
| | | 1770, | calcd for | 1.1 Hz, 1H), 5.14 (dq, J = 9.4, 6.3 Hz, 1H), |
| | | 1735, | C₂₈H₃₇N₂O₈, | 4.77 (ddd, J = 7.6, 5.2, 3.5 Hz, 1H), 4.26 (dd, |
| | | 1677, | 529.2544; | J = 9.4, 8.4 Hz, 1H), 3.91 (s, 3H), |
| | | 1492, | found, | 3.44-3.32 (m, 2H), 3.28 (ddd, J = 8.8, 5.9, 3.2 Hz, 1H), |
| | | 1193 | 529.2560 | 2.40 (s, 3H), 2.22-2.12 (m, 1H), |
| | | | | 2.12-2.01 (m, 1H), 1.96-1.83 (m, 1H), 1.71-1.58 (m, |
| | | | | 3H), 1.57-1.46 (m, 2H), 1.40-1.24 (m, |
| | | | | 2H), 1.33 (d, J = 6.3 Hz, 3H), 0.70 (t, J = 7.4 Hz, |
| | | | | 3H) |
| 27 | — | (Neat) | HRMS-ESI | ¹H NMR (400 MHz, CDCl₃) δ 8.71 (s, 1H), |
| | | 3380, | (m/z) | 8.35 (d, J = 5.5 Hz, 1H), 7.00 (d, J = 5.5 Hz, |
| | | 2941, | [M + H]⁺ | 1H), 4.89 (dq, J = 9.0, 6.3 Hz, 1H), 4.72 (ddd, |
| | | 1770, | calcd for | J = 7.6, 5.4, 3.4 Hz, 1H), 3.91 (s, 3H), 3.57 (s, |
| | | 1735, | C₂₁H₃₁N₂O₈, | 3H), 3.42 (s, 3H), 3.09 (t, J = 8.7 Hz, 1H), |
| | | 1504, | 439.2075; | 3.03 (ddd, J = 8.5, 5.9, 2.9 Hz, 1H), 2.40 (s, |
| | | 1194 | found, | 3H), 2.18-1.97 (m, 2H), 1.78 (ddt, J = 14.5, |
| | | | 439.2081 | 8.7, 5.7 Hz, 1H), 1.66-1.45 (m, 3H), |
| | | | | 1.42-1.33 (m, 1H), 1.37 (d, J = 6.3 Hz, 3H), |
| | | | | 1.28-1.15 (m, 1H) |
| 28 | — | (Neat) | HRMS-ESI | ¹H NMR (400 MHz, CDCl₃) δ 8.71 (s, 1H), |
| | | 3379, | (m/z) | 8.35 (d, J = 5.4 Hz, 1H), 7.00 (d, J = 5.5 Hz, |
| | | 2953, | [M + H]⁺ | 1H), 4.90 (dq, J = 9.3, 6.3 Hz, 1H), 4.71 (ddd, |
| | | 1771, | calcd for | J = 7.6, 5.5, 3.4 Hz, 1H), 3.91 (s, 3H), |
| | | 1734, | C₂₄H₃₇N₂O₈, | 3.64 (dd, J = 8.5, 6.4 Hz, 1H), 3.39 (s, 3H), |
| | | 1677, | 481.2544; | 3.30 (dd, J = 8.5, 6.5 Hz, 1H), 3.16 (dd, J = 9.3, 8.3 Hz, |
| | | 1504, | found, | 1H), 3.02 (ddd, J = 8.6, 5.9, 3.0 Hz, 1H), |
| | | 1196 | 481.2550 | 2.40 (s, 3H), 2.17-1.96 (m, 2H), |
| | | | | 1.90-1.71 (m, 2H), 1.65-1.54 (m, 2H), 1.52-1.43 (m, |
| | | | | 1H), 1.41-1.31 (m, 1H), 1.37 (d, J = 6.3 Hz, |
| | | | | 3H), 1.29-1.16 (m, 1H), 0.92 (d, J = 6.7 Hz, |
| | | | | 6H) |
| 29 | — | (Neat) | HRMS-ESI | ¹H NMR (400 MHz, CDCl₃) δ 8.59 (d, J = 7.4 Hz, |
| | | 3377, | (m/z) | 1H), 8.34 (d, J = 5.4 Hz, 1H), 7.01 (d, J = 5.5 Hz, |
| | | 2937, | [M + H]⁺ | 1H), 4.98 (dq, J = 9.2, 6.2 Hz, 1H), |
| | | 1770, | calcd for | 4.85 (ddd, J = 8.0, 6.9, 5.8 Hz, 1H), 3.91 (s, |
| | | 1740, | C₂₂H₃₁N₂O₈, | 3H), 3.86 (td, J = 7.7, 3.6 Hz, 1H), 3.59 (dd, J = 9.3, |
| | | 1676, | 451.2075; | 7.9 Hz, 1H), 2.40 (s, 3H), |
| | | 1504, | found, | 2.16-2.05 (m, 1H), 1.99-1.58 (m, 5H), 1.49-1.39 (m, |
| | | 1193 | 451.2084 | 7H), 1.36 (s, 3H), 1.33-1.23 (m, 1H) |
| 30 | — | — | HRMS-ESI | ¹H NMR (400 MHz, CDCl₃) δ 8.55 (d, J = 7.3 Hz, |
| | | | (m/z) | 1H), 8.29 (d, J = 5.4 Hz, 1H), 6.96 (d, J = 5.4 Hz, |
| | | | [M + H]⁺ | 1H), 5.75 (q, J = 6.4 Hz, 2H), |
| | | | calcd for | 4.93 (dq, J = 9.2, 6.3 Hz, 1H), 4.76 (ddd, J = 7.6, |
| | | | C₂₇H₄₃N₂O₈, | 5.2, 3.5 Hz, 1H), 3.91 (s, 3H), 3.44-3.20 (m, |
| | | | 523.3014; | 2H), 2.95 (t, J = 9.3 Hz, 1H), 2.28-2.13 (m, |
| | | | found, | 1H), 2.13-1.97 (m, 4H), 1.94-1.75 (m, |
| | | | 523.3027 | 1H), 1.72-1.58 (m, 1H), 1.58-1.46 (m, |
| | | | | 4H), 1.46-1.31 (m, 5H), 1.32-1.06 (m, |
| | | | | 4H), 0.91 (dd, J = 15.4, 7.0 Hz, 9H) |
| | | | | ¹³C NMR (101 MHz, CDCl₃) δ 172.53, |
| | | | | 170.20, 163.01, 160.24, 145.80, 143.83, |
| | | | | 142.60, 109.54, 89.53, 84.56, 79.45, 75.71, |
| | | | | 56.17, 51.72, 43.14, 34.31, 29.09, 27.28, |
| | | | | 26.92, 24.91, 21.96, 20.84, 20.14, 19.52, |
| | | | | 19.49, 18.48, 14.42 |
| 31 | — | — | HRMS-ESI | ¹H NMR (400 MHz, CDCl₃) δ 8.54 (d, J = 7.3 Hz, |
| | | | (m/z) | 1H), 8.30 (dd, J = 5.4, 0.9 Hz, 1H), |
| | | | [M + H]⁺ | 6.95 (d, J = 5.4 Hz, 1H), 5.75 (qd, J = 6.4, 0.9 Hz, |
| | | | calcd for | 2H), 5.03 (dq, J = 9.4, 6.2 Hz, 1H), 4.86 (t, J = 9.5 Hz, |
| | | | C₂₇H₄₁N₂O₉, | 1H), 4.81 (dt, J = 7.7, 4.4 Hz, 1H), |
| | | | 537.2807; | 3.91 (s, 3H), 2.59 (hept, J = 6.9 Hz, 1H), |
| | | | found, | 2.32-2.17 (m, 1H), 2.13-2.01 (m, 4H), |
| | | | 537.2823 | 1.76-1.64 (m, 1H), 1.64-1.44 (m, 4H), 1.25 (d, J = 3.7 Hz, |
| | | | | 3H), 1.22 (d, J = 6.3 Hz, 3H), |
| | | | | 1.20 (d, J = 7.0 Hz, 6H), 1.18-1.08 (m, 2H), |
| | | | | 0.85 (t, J = 6.9 Hz, 3H) |
| | | | | ¹³C NMR (101 MHz, CDCl₃) δ 176.17, |
| | | | | 172.58, 170.27, 163.02, 160.28, 145.78, |
| | | | | 143.97, 142.58, 109.55, 89.59, 76.39, 73.94, |
| | | | | 56.18, 51.66, 41.60, 34.36, 34.29, 26.91, |
| | | | | 24.78, 21.84, 20.87, 19.61, 19.01, 17.99, |
| | | | | 14.32 |
| 32 | — | (Neat) | HRMS-ESI | ¹H NMR (400 MHz, CDCl₃) δ 8.70 (d, J = 6.7 Hz, |
| | | 3344, | (m/z) | 1H), 8.28 (d, J = 5.3 Hz, 1H), |
| | | 2934, | [M + H]⁺ | 7.28-7.19 (m, 4H), 6.94 (d, J = 5.4 Hz, 1H), |
| | | 1749, | calcd for | 6.90-6.81 (m, 4H), 5.76 (d, J = 6.4 Hz, 1H), 5.73 (d, J = 6.4 Hz, |
| | | 1732, | C₃₅H₄₃N₂O₁₂, | 1H), 5.05 (dq, J = 9.1, 6.3 Hz, 1H), |
| | | 1681, | 683.2811; | 4.86 (d, J = 10.3 Hz, 1H), 4.69 (dt, J = 6.6, |
| | | 1512, | found, | 4.5 Hz, 1H), 4.59 (s, 2H), 4.55 (d, J = 10.3 Hz, |
| | | 1207, | 683.2819 | 1H), 3.95-3.83 (m, 1H), 3.90 (s, 3H), |
| | | 1033 | | 3.80 (d, J = 2.1 Hz, 6H), 3.62 (dd, J = 9.5, 7.9 Hz, |
| | | | | 1H), 3.54 (dd, J = 9.4, 1.5 Hz, 1H), |
| | | | | 3.49-3.36 (m, 3H), 2.58 (ddt, J = 14.8, 11.4, 3.5 Hz, |
| | | | | 1H), 2.06 (s, 3H), 2.02-1.94 (m, 1H), |
| | | | | 1.37 (d, J = 6.3 Hz, 3H) |
| 33 | — | (Neat) | HRMS-ESI | ¹H NMR (400 MHz, CDCl₃) δ 8.40 (d, J = 7.8 Hz, |
| | | 3376, | (m/z) | 1H), 8.28 (d, J = 5.4 Hz, 1H), 6.96 (d, J = 5.6 Hz, |
| | | 2937, | [M + H]⁺ | 1H), 5.75 (s, 2H), 4.98 (tt, J = 7.7, 5.7 Hz, |
| | | 1746, | calcd for | 1H), 4.87 (qd, J = 6.6, 5.9, 1.6 Hz, 1H), |
| | | 1676, | C₂₃H₃₃N₂O₉, | 3.92 (s, 3H), 3.88-3.80 (m, 1H), 3.60 (td, J = 8.5, |
| | | 1501, | 481.2181; | 7.8, 1.3 Hz, 1H), 2.18-2.09 (m, 1H), |
| | | 1196 | found, | 2.08 (s, 3H), 1.98-1.81 (m, 2H), |
| | | | 481.2181 | 1.81-1.57 (m, 3H), 1.50-1.40 (m, 7H), 1.37 (s, 3H), |
| | | | | 1.34-1.23 (m, 1H) |
| 34 | — | (Neat) | HRMS-ESI | ¹H NMR (400 MHz, CDCl₃) δ 8.52 (d, J = 7.3 Hz, |
| | | 3339, | (m/z) | 1H), 8.29 (d, J = 5.3 Hz, 1H), 6.95 (d, J = 5.4 Hz, |
| | | 2945, | [M + H]⁺ | 1H), 5.77 (d, J = 6.4 Hz, 1H), 5.73 (d, |
| | | 2877, | calcd for | J = 6.4 Hz, 1H), 4.87 (dq, J = 9.2, 6.3 Hz, |
| | | 1750, | C₂₆H₃₉N₂O₉, | 1H), 4.74 (ddd, J = 7.4, 5.1, 3.7 Hz, 1H), |
| | | 1727, | 523.265; | 4.30 (p, J = 4.7 Hz, 1H), 3.91 (s, 3H), 3.40 (s, 3H), |
| | | 1502, | found, | 3.29 (dd, J = 9.3, 8.2 Hz, 1H), 2.99 (ddd, J = 8.3, |
| | | 1202 | 523.2662 | 4.9, 3.2 Hz, 1H), 2.12-2.03 (m, 2H), |
| | | | | 2.07 (s, 3H), 1.83-1.40 (m, 13H), 1.37 (d, J = 6.3 Hz, |
| | | | | 3H), 1.35-1.25 (m, 1H) |
| 35 | — | (Neat) | HRMS-ESI | ¹H NMR (400 MHz, CDCl₃) δ 8.53 (d, J = 7.3 Hz, |
| | | 3345, | (m/z) | 1H), 8.29 (d, J = 5.4 Hz, 1H), 6.95 (d, J = 5.4 Hz, |
| | | 2944, | [M + H]⁺ | 1H), 5.77 (d, J = 6.4 Hz, 1H), 5.73 (d, |
| | | 1751, | calcd for | J = 6.4 Hz, 1H), 4.87 (dq, J = 9.2, 6.3 Hz, |
| | | 1728, | C₂₆H₃₉N₂O₉, | 1H), 4.76 (ddd, J = 7.4, 5.3, 3.4 Hz, 1H), |
| | | 1681, | 523.265; | 4.08 (td, J = 5.0, 2.7 Hz, 1H), 3.91 (s, 3H), 3.57 (s, |
| | | 1502, | found, | 3H), 3.16 (ddd, J = 8.9, 6.2, 3.0 Hz, 1H), |
| | | 1203, | 523.2658 | 3.04 (t, J = 8.8 Hz, 1H), 2.21-2.12 (m, 1H), |
| | | 1105 | | 2.10-2.00 (m, 1H), 2.07 (s, 3H), 1.84-1.42 (m, |
| | | | | 12H), 1.41-1.35 (m, 1H), 1.38 (d, J = 6.2 Hz, |
| | | | | 3H), 1.25-1.12 (m, 1H) |
| 36 | — | (Neat) | HRMS-ESI | ¹H NMR (400 MHz, CDCl₃) δ 8.55 (d, J = 7.3 Hz, |
| | | 3347, | (m/z) | 1H), 8.29 (d, J = 5.3 Hz, 1H), |
| | | 2941, | [M + H]⁺ | 7.29-7.23 (m, 2H), 6.98-6.86 (m, 4H), 5.77 (d, J = 6.4 Hz, |
| | | 1748, | calcd for | 1H), 5.74 (d, J = 6.4 Hz, 1H), 5.01 (dq, J = 9.3, |
| | | 1729, | C₃₁H₄₁N₂O₉, | 6.3 Hz, 1H), 4.79 (ddd, J = 7.3, 5.3, 3.4 Hz, |
| | | 1681, | 585.2807; | 1H), 4.34-4.27 (m, 1H), 4.12 (ddd, J = 8.6, |
| | | 1493, | found, | 5.8, 3.0 Hz, 1H), 3.91 (s, 3H), 3.55 (dd, J = 9.3, |
| | | 1198 | 585.2819 | 8.2 Hz, 1H), 2.21-2.10 (m, 1H), |
| | | | | 2.09-2.05 (m, 1H), 2.07 (s, 3H), 2.01-1.87 (m, |
| | | | | 1H), 1.72-1.36 (m, 12H), 1.43 (d, J = 6.3 Hz, |
| | | | | 3H), 1.36-1.24 (m, 1H) |
| 37 | — | (Neat) | HRMS-ESI | ¹H NMR (400 MHz, CDCl₃) δ 8.74 (d, J = 6.7 Hz, |
| | | 3374, | (m/z) | 1H), 8.31 (d, J = 5.3 Hz, 1H), |
| | | 2925, | [M + H]⁺ | 7.31-7.13 (m, 4H), 7.03-6.85 (m, 5H), 6.79-6.70 (m, |
| | | 1741, | calcd for | 2H), 5.77 (d, J = 6.4 Hz, 1H), 5.75 (d, J = 6.4 Hz, |
| | | 1675, | C₃₁H₃₅N₂O₁₀, | 1H), 5.37 (dq, J = 9.6, 6.3 Hz, 1H), |
| | | 1585, | 595.2286; | 4.77 (ddd, J = 6.7, 5.1, 4.0 Hz, 1H), 4.52 (dd, J = 9.7, |
| | | 1491, | found, | 8.6 Hz, 1H), 4.40-4.33 (m, 1H), |
| | | 1200 | 595.2297 | 4.01-3.88 (m, 1H), 3.92 (s, 3H), 3.82 (dd, J = 9.8, |
| | | | | 8.1 Hz, 1H), 3.61 (dd, J = 9.8, 1.5 Hz, 1H), |
| | | | | 3.45-3.37 (m, 1H), 2.71-2.59 (m, 1H), |
| | | | | 2.08 (s, 3H), 2.07-2.00 (m, 1H), 1.41 (d, J = 6.3 Hz, |
| | | | | 3H) |
| 38 | — | (Neat) | HRMS-ESI | ¹H NMR (400 MHz, CDCl₃) δ 8.55 (d, J = 7.3 Hz, |
| | | 3377, | (m/z) | 1H), 8.29 (d, J = 5.4 Hz, 1H), 6.96 (d, J = 5.4 Hz, |
| | | 2959, | [M + H]⁺ | 1H), 5.77 (d, J = 6.4 Hz, 2H), 5.73 (d, |
| | | 1739, | calcd for | J = 6.4 Hz, 1H), 4.98 (dq, J = 9.3, 6.3 Hz, |
| | | 1677, | C₂₈H₄₃N₂O₁₁, | 1H), 4.79 (ddd, J = 8.0, 4.9, 3.4 Hz, 1H), |
| | | 1501, | 583.2870; | 4.61 (ddd, J = 9.1, 6.4, 3.0 Hz, 1H), 4.09 (t, J = 6.8 Hz, |
| | | 1255, | found, | 2H), 3.91 (s, 3H), 3.50 (dd, J = 8.2, 6.5 Hz, |
| | | 1198 | 583.2861 | 1H), 3.34 (dd, J = 8.3, 6.3 Hz, 1H), |
| | | | | 3.31-3.24 (m, 1H), 2.14 (ddt, J = 16.3, 8.4, 4.8 Hz, |
| | | | | 1H), 2.09-2.01 (m, 1H), 2.07 (s, 3H), |
| | | | | 1.98-1.87 (m, 1H), 1.86-1.62 (m, 4H), |
| | | | | 1.62-1.47 (m, 2H), 1.40 (d, J = 6.3 Hz, 3H), |
| | | | | 1.28-1.16 (m, 1H), 0.97 (t, J = 7.5 Hz, 3H), 0.88 (d, J = 6.7 Hz, |
| | | | | 3H), 0.88 (d, J = 6.8 Hz, 3H) |
| 39 | 57-62 | — | HRMS-ESI | ¹H NMR (600 MHz, CDCl₃) δ 8.92 (d, J = 8.1 Hz, |
| | | | (m/z) | 1H), 8.40-8.36 (m, 1H), 7.02 (d, J = 5.4 Hz, |
| | | | [M + H]⁺ | 1H), 5.10 (dq, J = 9.7, 6.3 Hz, 1H), |
| | | | calcd for | 4.94 (ddd, J = 8.1, 3.8, 1.8 Hz, 1H), |
| | | | C₂₃H₃₅N₂O₇, | 3.99-3.88 (m, 5H), 3.59 (ddd, J = 11.2, 10.1, 3.0 Hz, |
| | | | 451.2439; | 1H), 3.45 (ddd, J = 11.5, 5.0, 3.5 Hz, 1H), |
| | | | found, | 2.42 (d, J = 1.0 Hz, 3H), 1.72-1.60 (m, 2H), |
| | | | 451.2446 | 1.57-1.49 (m, 1H), 1.47-1.35 (m, 4H), |
| | | | | 1.34-1.27 (m, 4H), 1.24-1.12 (m, 2H), 0.92 (d, J = 6.6 Hz, |
| | | | | 3H), 0.91 (d, J = 6.6 Hz, 3H) |
| 40 | — | (Neat) | HRMS-ESI | ¹H NMR (400 MHz, CDCl₃) δ 8.71 (d, J = 6.7 Hz, |
| | | 3342, | (m/z) | 1H), 8.30 (d, J = 5.4 Hz, 1H), |
| | | 2934, | [M + H]⁺ | 7.31-7.25 (m, 2H), 7.04-6.99 (m, 2H), 6.99-6.93 (m, |
| | | 1749, | calcd for | 2H), 5.77 (d, J = 6.4 Hz, 1H), 5.74 (d, J = 6.4 Hz, |
| | | 1681, | C₂₆H₃₃N₂O₁₀, | 1H), 5.20 (dq, J = 9.7, 6.3 Hz, 1H), |
| | | 1492, | 533.213; | 4.73 (ddd, J = 6.7, 5.1, 4.1 Hz, 1H), 4.27 (dd, J = 9.7, |
| | | 1203 | found, | 8.5 Hz, 1H), 3.96-3.86 (m, 1H), 3.92 (s, |
| | | | 533.2136 | 3H), 3.63 (dd, J = 9.7, 8.2 Hz, 1H), 3.54 (dd, J = 9.7, |
| | | | | 1.8 Hz, 1H), 3.45 (ddd, J = 12.4, 4.3, |
| | | | | 2.7 Hz, 1H), 3.35 (s, 3H), 3.34-3.27 (m, |
| | | | | 1H), 2.65-2.54 (m, 1H), 2.07 (s, 3H), |
| | | | | 2.05-1.95 (m, 1H), 1.34 (d, J = 6.3 Hz, 3H) |
| 41 | — | (Neat) | HRMS-ESI | ¹H NMR (400 MHz, CDCl₃) δ 8.71 (d, J = 6.7 Hz, |
| | | 3364, | (m/z) | 1H), 8.30 (d, J = 5.4 Hz, 1H), |
| | | 2935, | [M + H]⁺ | 7.31-7.20 (m, 2H), 7.04-6.99 (m, 2H), 6.98-6.90 (m, |
| | | 1739, | calcd for | 2H), 5.77 (d, J = 6.4 Hz, 1H), 5.74 (d, J = 6.3 Hz, |
| | | 1677, | C₂₈H₃₇N₂O₁₀, | 1H), 5.20 (dq, J = 9.7, 6.3 Hz, 1H), |
| | | 1492, | 561.2448; | 4.76-4.68 (m, 1H), 4.28 (dd, J = 9.7, 8.5 Hz, 1H), |
| | | 1201 | found, | 3.96-3.86 (m, 1H), 3.92 (s, 3H), 3.65 (dd, J = 9.7, |
| | | | 561.2457 | 8.5 Hz, 1H), 3.55-3.50 (m, 1H), |
| | | | | 3.50-3.35 (m, 4H), 2.66-2.53 (m, 1H), 2.07 (s, |
| | | | | 3H), 2.06-1.94 (m, 1H), 1.39-1.28 (m, |
| | | | | 2H), 1.33 (d, J = 6.4 Hz, 3H), 0.69 (t, J = 7.4 Hz, |
| | | | | 3H) |
| 42 | — | (Neat) | HRMS-ESI | ¹H NMR (400 MHz, CDCl₃) δ 8.73 (d, J = 6.7 Hz, |
| | | 3347, | (m/z) | 1H), 8.30 (d, J = 5.4 Hz, 1H), |
| | | 2934, | [M + H]⁺ | 7.31-7.25 (m, 2H), 6.98-6.91 (m, 4H), 5.77 (d, J = 6.3 Hz, |
| | | 1750, | calcd for | 1H), 5.74 (d, J = 6.4 Hz, 1H), 5.13 (dq, J = 9.8, |
| | | 1731, | C₂₈H₃₇N₂O₁₀, | 6.3 Hz, 1H), 4.73 (ddd, J = 6.7, 4.8, 3.8 Hz, |
| | | 1681, | 561.2443; | 1H), 4.25-4.16 (m, 1H), 3.94-3.90 (m, |
| | | 1493, | found, | 1H), 3.92 (s, 3H), 3.90-3.80 (m, 1H), |
| | | 1206 | 561.2455 | 3.70 (dd, J = 9.6, 8.1 Hz, 1H), 3.59-3.51 (m, 1H), |
| | | | | 3.52-3.40 (m, 2H), 3.39-3.30 (m, 1H), |
| | | | | 2.70-2.52 (m, 1H), 2.07 (s, 3H), 2.04-1.96 (m, |
| | | | | 1H), 1.49 (ddq, J = 14.4, 7.3, 4.2, 3.7 Hz, 2H), |
| | | | | 1.43 (d, J = 6.3 Hz, 3H), 0.83 (t, J = 7.4 Hz, |
| | | | | 3H) |
| 43 | — | (Neat) | HRMS-ESI | ¹H NMR (400 MHz, CDCl₃) δ 8.69 (d, J = 6.7 Hz, |
| | | 3359, | (m/z) | 1H), 8.29 (d, J = 5.3 Hz, 1H), 6.95 (d, J = 5.4 Hz, |
| | | 2955, | [M + H]⁺ | 1H), 5.76 (d, J = 6.7 Hz, 1H), 5.73 (d, |
| | | 1733, | calcd for | J = 6.4 Hz, 1H), 4.98 (dq, J = 8.7, 6.4 Hz, |
| | | 1680, | C₂₇H₄₃N₂O_{10,} | 1H), 4.72-4.61 (m, 1H), 3.91 (s, 3H), |
| | | 1205 | 555.2912; | 3.90-3.81 (m, 1H), 3.70 (dd, J = 8.4, 6.1 Hz, 1H), |
| | | | found, | 3.53 (dd, J = 9.6, 7.5 Hz, 1H), 3.46-3.09 (m, |
| | | | 555.2916 | 7H), 2.64-2.50 (m, 1H), 2.07 (s, 3H), |
| | | | | 2.01-1.91 (m, 1H), 1.88-1.71 (m, 2H), 1.37 (d, J = 6.3 Hz, |
| | | | | 3H), 0.95-0.87 (m, 12H) |
| 44 | — | (Neat) | HRMS-ESI | ¹H NMR (400 MHz, CDCl₃) δ 8.69 (d, J = 6.8 Hz, |
| | | 2976, | (m/z) | 1H), 8.31 (d, J = 5.4 Hz, 1H), |
| | | 1736, | [M + H]⁺ | 7.32-7.21 (m, 2H), 7.00-6.90 (m, 4H), 5.76 (d, J = 6.4 Hz, |
| | | 1676, | calcd for | 1H), 5.74 (d, J = 6.3 Hz, 1H), 5.25 (dq, J = 9.7, |
| | | 1492, | C₂₉H₃₇N₂O₁₁, | 6.4 Hz, 1H), 5.10-5.04 (m, 1H), |
| | | 1200 | 589.2392; | 4.75 (ddd, J = 7.0, 5.5, 4.0 Hz, 1H), 4.53 (dd, J = 9.7, |
| | | | found, | 8.6 Hz, 1H), 3.95-3.87 (m, 1H), 3.92 (s, |
| | | | 589.24 | 3H), 3.74 (dd, J = 9.8, 8.2 Hz, 1H), |
| | | | | 3.50-3.42 (m, 2H), 2.62-2.50 (m, 1H), |
| | | | | 2.24-2.13 (m, 1H), 2.07 (s, 3H), 2.06-1.98 (m, 1H), |
| | | | | 1.36 (d, J = 6.3 Hz, 3H), 0.92 (d, J = 7.0 Hz, |
| | | | | 3H), 0.85 (d, J = 7.0 Hz, 3H) |
| 45 | — | (Neat) | HRMS-ESI | ¹H NMR (400 MHz, CDCl₃) δ 8.72 (d, J = 6.7 Hz, |
| | | 2955, | (m/z) | 1H), 8.30 (d, J = 5.4 Hz, 1H), |
| | | 1742, | [M + H]⁺ | 7.30-7.19 (m, 2H), 7.06-6.83 (m, 4H), 5.77 (d, J = 6.4 Hz, |
| | | 1677, | calcd for | 1H), 5.74 (d, J = 6.3 Hz, 1H), 5.21 (dq, J = 9.6, |
| | | 1492, | C₂₉H₃₉N₂O₁₀, | 6.3 Hz, 1H), 4.76-4.69 (m, 1H), |
| | | 1201 | 575.2599; | 4.33-4.22 (m, 1H), 3.96-3.85 (m, 1H), 3.92 (s, |
| | | | found, | 3H), 3.69-3.61 (m, 1H), 3.53 (dd, J = 9.7, |
| | | | 575.2604 | 1.8 Hz, 1H), 3.46-3.40 (m, 1H), |
| | | | | 3.40-3.33 (m, 1H), 3.28 (dd, J = 8.9, 6.4 Hz, 1H), |
| | | | | 3.22 (dd, J = 8.9, 6.4 Hz, 1H), 2.67-2.55 (m, 1H), |
| | | | | 2.07 (s, 3H), 2.06-1.96 (m, 1H), 1.57 (hept, |
| | | | | J = 6.6 Hz, 1H), 1.32 (d, J = 6.3 Hz, 3H), |
| | | | | 0.70 (d, J = 6.7 Hz, 3H), 0.65 (d, J = 6.7 Hz, 3H) |
| 46 | — | (Neat) | HRMS-ESI | ¹H NMR (400 MHz, CDCl₃) δ 8.72 (d, J = 6.7 Hz, |
| | | 2956, | (m/z) | 1H), 8.30 (d, J = 5.3 Hz, 1H), |
| | | 1733, | [M + H]⁺ | 7.31-7.24 (m, 2H), 7.00-6.87 (m, 4H), 5.77 (d, J = 6.3 Hz, |
| | | 1680, | calcd for | 1H), 5.74 (d, J = 6.4 Hz, 1H), 5.14 (dq, J = 9.7, |
| | | 1493, | C₂₉H₃₉N₂O₁₀, | 6.3 Hz, 1H), 4.73 (ddd, J = 6.6, 4.9, 3.9 Hz, |
| | | 1204 | 575.2599; | 1H), 4.26-4.13 (m, 1H), 3.96-3.86 (m, |
| | | | found, | 1H), 3.92 (s, 3H), 3.73-3.66 (m, 2H), |
| | | | 575.2610 | 3.51-3.45 (m, 1H), 3.42 (dd, J = 9.8, 8.6 Hz, 1H), |
| | | | | 3.38-3.29 (m, 2H), 2.66-2.55 (m, 1H), |
| | | | | 2.07 (s, 3H), 2.04-1.95 (m, 1H), 1.73 (hept, J = 6.7 Hz, |
| | | | | 1H), 1.42 (d, J = 6.4 Hz, 3H), 0.83 (d, |
| | | | | J = 6.7 Hz, 3H), 0.79 (d, J = 6.7 Hz, 3H) |
| 47 | — | (Neat) | HRMS-ESI | ¹H NMR (400 MHz, CDCl₃) δ 8.94 (d, J = 6.9 Hz, |
| | | 3375, | (m/z) | 1H), 8.36 (d, J = 5.4 Hz, 1H), |
| | | 2937, | [M + H]⁺ | 7.31-7.15 (m, 4H), 7.07-6.86 (m, 5H), 6.78-6.70 (m, |
| | | 1769, | calcd for | 2H), 5.36 (dq, J = 9.7, 6.3 Hz, 1H), 4.74 (ddd, |
| | | 1740, | C₃₀H₃₃N₂O₉, | J = 6.9, 5.0, 4.0 Hz, 1H), 4.51 (dd, J = 9.7, 8.6 Hz, |
| | | 1491, | 565.2181; | 1H), 4.41-4.33 (m, 1H), 3.97-3.87 (m, |
| | | 1198 | found, | 1H), 3.92 (s, 3H), 3.80 (dd, J = 9.9, 8.0 Hz, |
| | | | 565.2187 | 1H), 3.61 (dd, J = 9.8, 1.4 Hz, 1H), |
| | | | | 3.44-3.36 (m, 1H), 2.68-2.56 (m, 1H), 2.40 (s, |
| | | | | 3H), 2.05-1.95 (m, 1H), 1.40 (d, J = 6.3 Hz, |
| | | | | 3H) |
| 48 | — | (Neat) | HRMS-ESI | ¹H NMR (400 MHz, CDCl₃) δ 8.90 (d, J = 6.9 Hz, |
| | | 3375, | (m/z) | 1H), 8.35 (d, J = 5.4 Hz, 1H), |
| | | 2936, | [M + H]⁺ | 7.31-7.24 (m, 2H), 7.04-6.93 (m, 4H), 5.20 (dq, J = 9.7, |
| | | 1769, | calcd for | 6.3 Hz, 1H), 4.69 (ddd, J = 7.0, 5.1, 4.0 Hz, |
| | | 1737, | C₂₅H₃₁N₂O₉, | 1H), 4.26 (dd, J = 9.7, 8.4 Hz, 1H), |
| | | 1675, | 503.2024; | 3.91 (s, 4H), 3.66-3.51 (m, 2H), 3.47-3.41 (m, |
| | | 1492, | found, | 1H), 3.35 (s, 3H), 3.33-3.26 (m, 1H), |
| | | 1196 | 503.2033 | 2.62-2.51 (m, 1H), 2.40 (s, 3H), 2.02-1.91 (m, |
| | | | | 1H), 1.33 (d, J = 6.4 Hz, 3H) |
| 49 | — | — | HRMS-ESI | ¹H NMR (400 MHz, CDCl₃) δ 8.50 (d, J = 7.2 Hz, |
| | | | (m/z) | 1H), 8.40 (dd, J = 4.5, 1.3 Hz, 1H), |
| | | | [M + H]⁺ | 7.53 (dd, J = 8.4, 1.3 Hz, 1H), 7.43 (dd, J = 8.4, 4.5 Hz, |
| | | | calcd for | 1H), 7.34-7.24 (m, 2H), 6.99-6.88 (m, |
| | | | C₃₁H₄₁N₂O₇, | 3H), 5.93-5.76 (m, 2H), 5.15 (dq, J = 9.1, |
| | | | 553.2908; | 6.3 Hz, 1H), 4.83 (ddd, J = 7.3, 5.2, 3.5 Hz, |
| | | | found, | 1H), 4.12 (t, J = 9.1 Hz, 1H), 2.30-2.14 (m, |
| | | | 553.2910 | 2H), 2.12 (s, 3H), 1.98-1.82 (m, 1H), |
| | | | | 1.78-1.57 (m, 8H), 1.57-1.44 (m, 5H), 1.32 (d, J = 8.7 Hz, |
| | | | | 1H), 1.28 (d, J = 6.3 Hz, 3H), |
| | | | | 1.19 (ddd, J = 13.5, 9.4, 4.9 Hz, 1H), |
| | | | | 1.05-0.94 (m, 2H) |
| | | | | ¹³C NMR (101 MHz, CDCl₃) δ 172.61, |
| | | | | 169.69, 163.07, 159.63, 153.26, 143.35, |
| | | | | 140.40, 129.57, 127.40, 127.08, 120.83, |
| | | | | 115.42, 87.30, 82.49, 75.35, 51.99, 42.14, |
| | | | | 38.68, 37.55, 33.56, 32.10, 27.37, 27.11, |
| | | | | 25.10, 25.04, 24.64, 21.86, 20.90, 18.74 |
| 50 | — | — | HRMS-ESI | ¹H NMR (400 MHz, CDCl₃) δ 8.56 (d, J = 7.3 Hz, |
| | | | (m/z) | 1H), 8.31 (d, J = 5.4 Hz, 1H), |
| | | | [M + H]⁺ | 7.33-7.27 (m, 2H), 6.98-6.89 (m, 4H), 5.77 (d, J = 6.4 Hz, |
| | | | calcd for | 1H), 5.74 (d, J = 6.4 Hz, 1H), 5.13 (dt, J = 9.1, |
| | | | C₃₂H₄₃N₂O₈, | 6.3 Hz, 1H), 4.85-4.74 (m, 1H), 4.12 (t, |
| | | | 583.3014; | J = 9.1 Hz, 1H), 3.92 (s, 3H), 2.29-2.16 (m, |
| | | | found, | 1H), 2.16-2.09 (m, 1H), 2.08 (s, 3H), |
| | | | 583.3021 | 1.89 (dq, J = 12.5, 7.5, 6.1 Hz, 1H), |
| | | | | 1.76-1.60 (m, 6H), 1.57-1.40 (m, 6H), 1.30 (s, 2H), |
| | | | | 1.28 (d, J = 6.3 Hz, 3H), 1.19 (ddd, J = 13.7, |
| | | | | 9.3, 5.2 Hz, 1H), 1.06-0.91 (m, 2H) |
| | | | | ¹³C NMR (101 MHz, CDCl₃) δ 172.55, |
| | | | | 170.29, 163.05, 160.28, 159.64, 145.83, |
| | | | | 143.92, 142.61, 129.57, 120.81, 115.42, |
| | | | | 109.55, 89.60, 82.46, 75.32, 56.18, 51.91, |
| | | | | 42.14, 38.65, 37.54, 33.56, 32.09, 27.33, |
| | | | | 25.10, 25.04, 24.58, 21.92, 20.89, 18.72 |
| 51 | — | — | HRMS-ESI | ¹H NMR (400 MHz, CDCl₃) δ 8.57 (d, J = 7.3 Hz, |
| | | | (m/z) | 1H), 8.30 (d, J = 5.4 Hz, 1H), |
| | | | [M + H]⁺ | 7.37-7.21 (m, 2H), 7.04-6.83 (m, 4H), 5.91-5.73 (m, |
| | | | calcd for | 2H), 5.22-5.08 (m, 1H), 4.87-4.70 (m, |
| | | | C₃₄H₄₇N₂O₉, | 1H), 4.18-4.06 (m, 3H), 3.91 (s, 3H), |
| | | | 627.3276; | 3.59 (q, J = 7.0 Hz, 2H), 2.29-2.15 (m, 1H), |
| | | | found, | 2.15-2.02 (m, 1H), 1.88 (dd, J = 14.2, 6.6 Hz, |
| | | | 627.3285 | 1H), 1.75-1.60 (m, 5H), 1.58-1.41 (m, |
| | | | | 7H), 1.30 (s, 2H), 1.28 (d, J = 6.3 Hz, 3H), |
| | | | | 1.23 (t, J = 7.0 Hz, 3H), 1.18 (dt, J = 9.4, 4.7 Hz, |
| | | | | 1H), 1.04-0.93 (m, 2H) |
| | | | | ¹³C NMR (101 MHz, CDCl₃) δ 172.51, |
| | | | | 170.06, 163.00, 160.19, 159.64, 145.88, |
| | | | | 143.88, 142.46, 129.57, 120.82, 115.42, |
| | | | | 109.66, 89.59, 82.47, 75.33, 67.80, 67.20, |
| | | | | 56.22, 51.90, 42.13, 38.66, 37.54, 33.57, |
| | | | | 32.09, 27.35, 25.10, 25.04, 24.59, 21.91, |
| | | | | 18.73, 15.02 |
| 52 | — | — | HRMS-ESI | ¹H NMR (400 MHz, CDCl₃) δ 8.74 (s, 1H), |
| | | | (m/z) | 8.36 (d, J = 5.4 Hz, 1H), 7.34-7.22 (m, 2H), |
| | | | [M + H]⁺ | 7.01 (d, J = 5.5 Hz, 1H), 6.93 (dd, J = 11.7, |
| | | | calcd for | 7.6 Hz, 3H), 5.15 (dt, J = 9.2, 6.4 Hz, 1H), |
| | | | C₃₁H₄₁N₂O₇, | 4.86-4.69 (m, 1H), 4.11 (t, J = 9.1 Hz, 1H), |
| | | | 553.2908; | 3.91 (s, 3H), 2.40 (s, 3H), 2.26-2.14 (m, |
| | | | found, | 1H), 2.13-2.01 (m, 1H), 1.97-1.82 (m, |
| | | | 553.2923 | 1H), 1.78-1.60 (m, 5H), 1.57-1.41 (m, |
| | | | | 7H), 1.30 (s, 2H), 1.26 (d, J = 6.3 Hz, 3H), |
| | | | | 1.23-1.13 (m, 1H), 1.03-0.94 (m, 2H) |
| | | | | ¹³C NMR (101 MHz, CDCl₃) δ 172.42, |
| | | | | 168.93, 162.49, 159.64, 159.42, 146.78, |
| | | | | 141.59, 137.44, 129.56, 120.80, 115.42, |
| | | | | 109.74, 82.48, 75.32, 56.29, 51.69, 42.12, |
| | | | | 38.70, 37.55, 33.56, 32.10, 27.40, 27.23, |
| | | | | 25.10, 25.04, 24.64, 21.81, 20.77, 18.71 |
| 53 | — | — | HRMS-ESI | ¹H NMR (400 MHz, CDCl₃) δ 8.54 (d, J = 7.3 Hz, |
| | | | (m/z) | 1H), 8.29 (d, J = 5.4 Hz, 1H), 6.96 (d, J = 5.4 Hz, |
| | | | [M + H]⁺ | 1H), 6.01-5.84 (m, 1H), 5.77 (d, J = 6.4 Hz, |
| | | | calcd for | 1H), 5.74 (d, J = 6.4 Hz, 1H), |
| | | | C₂₉H₄₃N₂O₈, | 5.28 (dd, J = 17.2, 1.6 Hz, 1H), 5.17 (dd, J = 10.4, |
| | | | 547.3014; | 1.4 Hz, 1H), 4.97 (dq, J = 9.1, 6.3 Hz, 1H), |
| | | | found, | 4.75 (ddd, J = 7.5, 5.4, 3.4 Hz, 1H), |
| | | | 547.3027 | 4.16-4.01 (m, 2H), 3.91 (s, 3H), 3.02 (t, J = 9.2 Hz, |
| | | | | 1H), 2.16 (ddt, J = 10.9, 7.0, 3.6 Hz, 1H), |
| | | | | 2.07 (s, 4H), 1.97-1.83 (m, 1H), 1.83-1.72 (m, |
| | | | | 1H), 1.68 (ddd, J = 11.5, 7.8, 3.7 Hz, 1H), |
| | | | | 1.65-1.43 (m, 10H), 1.39 (d, J = 6.3 Hz, |
| | | | | 3H), 1.23 (td, J = 12.1, 9.8, 4.5 Hz, 3H), |
| | | | | 1.14-0.99 (m, 2H) |
| | | | | ¹³C NMR (101 MHz, CDCl₃) δ 172.57, |
| | | | | 170.24, 163.02, 160.25, 145.81, 143.85, |
| | | | | 142.60, 134.33, 116.84, 109.54, 89.55, 85.45, |
| | | | | 75.46, 73.86, 56.18, 51.83, 42.16, 38.57, |
| | | | | 37.38, 33.84, 32.03, 27.26, 25.11, 25.04, |
| | | | | 24.64, 22.06, 20.87, 18.50 |
| 54 | — | — | HRMS-ESI | ¹H NMR (400 MHz, CDCl₃) δ 8.53 (d, J = 7.3 Hz, |
| | | | (m/z) | 1H), 8.30 (d, J = 5.4 Hz, 1H), 6.95 (d, J = 5.4 Hz, |
| | | | [M + H]⁺ | 1H), 5.77 (d, J = 6.4 Hz, 1H), 5.74 (d, |
| | | | calcd for | J = 6.4 Hz, 1H), 5.04 (dq, J = 9.5, 6.2 Hz, |
| | | | C₃₀H₄₅N₂O₉, | 1H), 4.88-4.73 (m, 2H), 3.91 (s, 3H), |
| | | | 577.3120; | 2.58 (hept, J = 7.0 Hz, 1H), 2.30-2.16 (m, 1H), |
| | | | found, | 2.08 (s, 4H), 1.85 (dt, J = 14.1, 7.8 Hz, 1H), |
| | | | 577.3122 | 1.79-1.64 (m, 3H), 1.60-1.44 (m, 8H), |
| | | | | 1.27 (d, J = 8.0 Hz, 2H), 1.22 (d, J = 6.3 Hz, 3H), |
| | | | | 1.20 (dd, J = 7.0, 1.3 Hz, 6H), 1.18-1.10 (m, |
| | | | | 2H), 1.00 (dt, J = 18.3, 7.2 Hz, 2H) |
| | | | | ¹³C NMR (101 MHz, CDCl₃) δ 176.23, |
| | | | | 172.65, 170.30, 163.01, 160.28, 145.79, |
| | | | | 143.97, 142.59, 109.54, 89.60, 73.84, 56.19, |
| | | | | 51.73, 40.88, 38.41, 37.17, 34.32, 33.69, |
| | | | | 32.10, 27.16, 26.93, 25.09, 25.02, 24.53, |
| | | | | 21.99, 20.89, 19.07, 19.02, 17.99 |
| 55 | — | — | HRMS-ESI | ¹H NMR (400 MHz, CDCl₃) δ 8.54 (d, J = 7.4 Hz, |
| | | | (m/z) | 1H), 8.30 (d, J = 5.4 Hz, 1H), 6.95 (d, J = 5.4 Hz, |
| | | | [M + H]⁺ | 1H), 5.84 (d, J = 6.4 Hz, 1H), 5.82 (d, |
| | | | calcd for | J = 6.4 Hz, 1H), 5.15-4.96 (m, 1H), 4.83 (t, |
| | | | C₃₂H₄₉N₂O₁₀, | J = 9.5 Hz, 1H), 4.80-4.75 (m, 1H), 4.10 (s, |
| | | | 621.3382; | 2H), 3.91 (s, 3H), 3.59 (q, J = 7.0 Hz, 2H), |
| | | | found, | 2.58 (hept, J = 6.9 Hz, 1H), 2.29-2.16 (m, |
| | | | 621.3389 | 1H), 2.06 (d, J = 5.6 Hz, 1H), 1.84 (dd, J = 13.5, |
| | | | | 7.9 Hz, 1H), 1.80-1.60 (m, 4H), |
| | | | | 1.58-1.43 (m, 7H), 1.27 (d, J = 8.3 Hz, 2H), |
| | | | | 1.25-1.21 (m, 6H), 1.21-1.18 (m, 8H), |
| | | | | 1.07-0.95 (m, 2H) |
| | | | | ¹³C NMR (101 MHz, CDCl₃) δ 176.23, |
| | | | | 172.61, 170.07, 162.96, 160.19, 145.84, |
| | | | | 143.92, 142.44, 109.65, 89.58, 73.85, 67.79, |
| | | | | 67.20, 56.22, 51.72, 40.87, 38.41, 37.17, |
| | | | | 34.32, 33.69, 32.10, 26.94, 25.09, 25.02, |
| | | | | 24.53, 21.99, 19.07, 19.02, 17.99, 15.01 |
| 56 | — | — | HRMS-ESI | ¹H NMR (400 MHz, CDCl₃) δ 8.70 (s, 1H), |
| | | | (m/z) | 8.36 (d, J = 5.4 Hz, 1H), 7.01 (d, J = 5.5 Hz, |
| | | | [M + H]⁺ | 1H), 5.11-4.99 (m, 1H), 4.82 (t, J = 9.5 Hz, |
| | | | calcd for | 1H), 4.77 (ddd, J = 8.0, 5.3, 3.5 Hz, 1H), |
| | | | C₂₉H₄₃N₂O₈, | 3.91 (s, 3H), 2.58 (hept, J = 7.0 Hz, 1H), 2.40 (s, |
| | | | 547.3014; | 3H), 2.26-2.15 (m, 1H), 2.04 (d, J = 5.2 Hz, |
| | | | found, | 1H), 1.91-1.79 (m, 1H), 1.79-1.63 (m, |
| | | | 547.3028 | 3H), 1.64-1.59 (m, 2H), 1.57-1.44 (m, |
| | | | | 6H), 1.34-1.25 (m, 1H), 1.24-1.12 (m, |
| | | | | 12H), 1.07-0.95 (m, 2H) |
| | | | | ¹³C NMR (101 MHz, CDCl₃) δ 176.23, |
| | | | | 172.53, 168.93, 162.45, 159.43, 146.74, |
| | | | | 141.60, 137.46, 109.74, 73.84, 56.29, 51.50, |
| | | | | 40.86, 38.45, 37.17, 34.32, 33.69, 32.10, |
| | | | | 27.21, 26.97, 25.09, 25.02, 24.58, 21.90, |
| | | | | 20.77, 19.07, 19.02, 17.97 |
| 57 | — | — | HRMS-ESI | ¹H NMR (400 MHz, CDCl₃) δ 8.54 (d, J = 7.2 Hz, |
| | | | (m/z) | 1H), 8.30 (d, J = 5.4 Hz, 1H), 6.95 (d, J = 5.4 Hz, |
| | | | [M + H]⁺ | 1H), 5.77 (d, J = 6.4 Hz, 2H), 5.74 (d, |
| | | | calcd for | J = 6.4 Hz, 2H), 5.05-4.86 (m, 1H), |
| | | | C₂₉H₄₅N₂O₈, | 4.81-4.63 (m, 1H), 3.91 (s, 3H), 3.58-3.41 (m, |
| | | | 549.3170; | 2H), 2.93 (t, J = 9.2 Hz, 1H), 2.14 (d, J = 3.5 Hz, |
| | | | found, | 1H), 2.07 (s, 3H), 2.06-1.99 (m, 1H), |
| | | | 549.3171 | 1.91 (s, 1H), 1.82-1.64 (m, 2H), 1.60 (d, J = 7.0 Hz, |
| | | | | 3H), 1.57-1.43 (m, 7H), 1.38 (d, J = 6.3 Hz, |
| | | | | 3H), 1.27-1.15 (m, 3H), |
| | | | | 1.15-1.00 (m, 2H), 0.94 (t, J = 7.4 Hz, 3H) |
| | | | | ¹³C NMR (101 MHz, CDCl₃) δ 172.62, |
| | | | | 170.28, 163.02, 160.25, 145.81, 142.67, |
| | | | | 109.50, 89.62, 85.24, 75.70, 74.67, 56.17, |
| | | | | 51.84, 42.28, 38.43, 37.38, 33.90, 32.05, |
| | | | | 27.24, 25.15, 25.07, 23.40, 22.10, 20.89, |
| | | | | 18.43, 10.75 |
| 58 | — | — | HRMS-ESI | ¹H NMR (400 MHz, CDCl₃) δ 8.72 (s, 1H), |
| | | | (m/z) | 8.35 (d, J = 5.4 Hz, 1H), 7.00 (d, J = 5.5 Hz, |
| | | | [M + H]⁺ | 1H), 4.94 (dq, J = 9.3, 6.2 Hz, 1H), 4.72 (ddd, |
| | | | calcd for | J = 8.0, 5.3, 3.4 Hz, 1H), 3.91 (s, 3H), |
| | | | C₂₈H₄₃N₂O₇, | 3.49 (ddt, J = 24.1, 8.5, 6.7 Hz, 2H), 2.92 (t, J = 9.2 Hz, |
| | | | 519.3065; | 1H), 2.40 (s, 3H), 2.22-2.09 (m, |
| | | | found, | 1H), 2.04 (d, J = 6.7 Hz, 1H), 1.96-1.84 (m, |
| | | | 519.3069 | 1H), 1.72 (ddt, J = 28.1, 17.0, 8.3 Hz, 2H), |
| | | | | 1.64-1.59 (m, 3H), 1.58-1.44 (m, 8H), |
| | | | | 1.43-1.39 (m, 1H), 1.37 (d, J = 6.3 Hz, 3H), |
| | | | | 1.20 (ddd, J = 14.4, 10.1, 4.5 Hz, 3H), |
| | | | | 1.14-1.01 (m, 2H), 0.93 (t, J = 7.4 Hz, 3H) |
| | | | | ¹³C NMR (101 MHz, CDCl₃) δ, 172.50, |
| | | | | 168.92, 162.45, 159.39, 146.76, 141.64, |
| | | | | 137.41, 109.69, 85.25, 75.70, 74.65, 56.27, |
| | | | | 51.62, 42.26, 38.48, 37.38, 33.90, 32.06, |
| | | | | 27.29, 25.15, 25.07, 24.73, 23.39, 22.01, |
| | | | | 20.77, 18.42, 10.75 |
| 59 | — | — | HRMS-ESI | ¹H NMR (400 MHz, CDCl₃) δ 8.72 (s, 1H), |
| | | | (m/z) | 8.35 (d, J = 5.5 Hz, 1H), 7.00 (d, J = 5.5 Hz, |
| | | | [M + H]⁺ | 1H), 4.93 (dq, J = 9.2, 6.3 Hz, 1H), 4.72 (ddd, |
| | | | calcd for | J = 7.8, 5.5, 3.4 Hz, 1H), 3.91 (s, 3H), 3.44 (s, |
| | | | C₂₆H₃₉N₂O₇, | 3H), 2.85 (t, J = 9.2 Hz, 1H), 2.40 (s, 3H), |
| | | | 491.2759; | 2.22-2.09 (m, 1H), 2.09-1.97 (m, 1H), |
| | | | found, | 1.88 (s, 1H), 1.82-1.65 (m, 2H), 1.61 (d, J = 3.3 Hz, |
| | | | 491.2752 | 2H), 1.57-1.45 (m, 6H), 1.43-1.33 (m, |
| | | | | 4H), 1.29-1.15 (m, 3H), 1.15-0.99 (m, 3H) |
| | | | | ¹³C NMR (101 MHz, CDCl₃) δ 172.53, |
| | | | | 168.92, 162.45, 159.40, 146.76, 141.63, |
| | | | | 137.42, 109.71, 87.12, 75.35, 60.39, 56.28, |
| | | | | 51.54, 42.26, 38.48, 37.32, 33.90, 32.07, |
| | | | | 27.28, 27.01, 25.17, 25.09, 24.70, 22.12, |
| | | | | 20.77, 18.39 |
| 60 | — | — | HRMS-ESI | ¹H NMR (400 MHz, CDCl₃) δ 8.50 (d, J = 7.4 Hz, |
| | | | (m/z) | 1H), 8.28 (d, J = 5.4 Hz, 1H), |
| | | | [M + H]⁺ | 7.30-7.24 (m, 2H), 7.18 (t, J = 7.3 Hz, 1H), 7.13 (d, J = 7.0 Hz, |
| | | | calcd for | 2H), 6.94 (d, J = 5.4 Hz, 1H), 5.76 (d, |
| | | | C₃₁H₄₁N₂O₉, | J = 6.4 Hz, 1H), 5.73 (d, J = 6.4 Hz, 1H), |
| | | | 585.2807; | 5.17-5.05 (m, 1H), 4.98 (t, J = 9.5 Hz, 1H), |
| | | | found, | 4.84-4.74 (m, 1H), 3.91 (s, 3H), 2.78 (dd, J = 13.6, |
| | | | 585.2814 | 3.9 Hz, 1H), 2.56 (hept, J = 6.9 Hz, 1H), |
| | | | | 2.23 (dd, J = 13.6, 11.1 Hz, 1H), |
| | | | | 2.20-2.13 (m, 1H), 2.07 (s, 3H), 2.04-1.96 (m, 1H), |
| | | | | 1.89 (d, J = 9.6 Hz, 1H), 1.59-1.50 (m, 1H), |
| | | | | 1.39 (d, J = 3.7 Hz, 2H), 1.35 (d, J = 6.3 Hz, |
| | | | | 2H), 1.27 (d, J = 6.2 Hz, 3H), 1.20 (d, J = 7.0 Hz, |
| | | | | 6H), 0.96 (d, J = 5.2 Hz, 1H) |
| | | | | ¹³C NMR (101 MHz, CDCl₃) δ 176.27, |
| | | | | 172.63, 170.28, 163.00, 160.28, 145.77, |
| | | | | 143.98, 142.52, 140.06, 128.98, 128.35, |
| | | | | 126.00, 109.55, 89.59, 73.66, 56.18, 51.62, |
| | | | | 43.56, 38.11, 34.23, 27.07, 25.88, 24.28, |
| | | | | 21.90, 20.88, 19.04, 19.00, 18.00 |
| 61 | — | — | HRMS-ESI | ¹H NMR (400 MHz, CDCl₃) δ 8.54 (d, J = 7.3 Hz, |
| | | | (m/z) | 1H), 8.30 (d, J = 5.4 Hz, 1H), 6.95 (d, J = 5.4 Hz, |
| | | | [M + H]⁺ | 1H), 5.77 (d, J = 6.4 Hz, 1H), 5.74 (d, |
| | | | calcd for | J = 6.4 Hz, 1H), 4.93 (dq, J = 9.2, 6.3 Hz, |
| | | | C₂₇H₄₁N₂O₈, | 1H), 4.79-4.71 (m, 1H), 3.91 (s, 3H), |
| | | | 521.2857; | 3.45 (s, 3H), 2.86 (t, J = 9.2 Hz, 1H), |
| | | | found, | 2.27-2.11 (m, 1H), 2.07 (s, 3H), 2.04 (d, J = 8.0 Hz, |
| | | | 521.2869 | 1H), 1.89 (d, J = 12.6 Hz, 1H), 1.74 (ddd, J = 14.2, |
| | | | | 11.4, 5.6 Hz, 2H), 1.66-1.60 (m, 2H), |
| | | | | 1.58-1.45 (m, 7H), 1.44-1.34 (m, 4H), |
| | | | | 1.31-1.24 (m, 1H), 1.24-1.16 (m, 2H), |
| | | | | 1.13-1.01 (m, 2H) |
| | | | | ¹³C NMR (101 MHz, CDCl₃) δ 172.66, |
| | | | | 170.28, 163.03, 160.27, 145.81, 143.90, |
| | | | | 142.65, 109.51, 89.61, 87.11, 75.36, 60.41, |
| | | | | 56.17, 51.76, 42.29, 38.44, 37.31, 33.90, |
| | | | | 32.06, 27.24, 26.96, 25.17, 25.09, 24.64, |
| | | | | 22.22, 20.89, 18.40 |
| 62 | — | — | HRMS-ESI | ¹H NMR (400 MHz, CDCl₃) δ 8.54 (d, J = 7.4 Hz, |
| | | | (m/z) | 1H), 8.28 (d, J = 5.4 Hz, 1H), |
| | | | [M + H]⁺ | 7.37-7.19 (m, 4H), 7.18-7.09 (m, 3H), 6.96 (dd, J = 15.1, |
| | | | calcd for | 6.7 Hz, 4H), 5.76 (d, J = 6.4 Hz, 1H), |
| | | | C₃₃H₃₉N₂O₈, | 5.73 (d, J = 6.4 Hz, 1H), 5.20 (dq, J = 9.1, 6.3 Hz, |
| | | | 591.2701; | 1H), 4.79 (ddd, J = 7.5, 5.4, 3.4 Hz, 1H), |
| | | | found, | 4.28 (t, J = 9.2 Hz, 1H), 3.98-3.82 (m, 3H), |
| | | | 591.2709 | 3.12 (dd, J = 13.4, 3.4 Hz, 1H), |
| | | | | 2.25-2.14 (m, 2H), 2.06 (s, 3H), 2.05-1.99 (m, 1H), |
| | | | | 1.59 (dq, J = 11.7, 5.9, 5.2 Hz, 1H), |
| | | | | 1.49-1.38 (m, 2H), 1.35-1.25 (m, 6H), |
| | | | | 1.07-0.98 (m, 1H) |
| | | | | ¹³C NMR (101 MHz, CDCl₃) δ 172.55, |
| | | | | 170.26, 163.06, 160.28, 159.37, 145.83, |
| | | | | 143.91, 142.53, 140.50, 129.69, 129.04, |
| | | | | 128.25, 125.84, 121.08, 115.38, 109.62, |
| | | | | 89.55, 81.96, 75.14, 56.20, 51.73, 45.43, |
| | | | | 38.64, 27.13, 26.14, 24.36, 21.93, 20.88, |
| | | | | 18.73 |
| 63 | — | — | HRMS-ESI | ¹H NMR (400 MHz, CDCl₃) δ 8.54 (d, J = 7.4 Hz, |
| | | | (m/z) | 1H), 8.28 (d, J = 5.4 Hz, 1H), |
| | | | [M + H]⁺ | 7.35-7.21 (m, 5H), 7.21-7.07 (m, 3H), 7.03-6.90 (m, |
| | | | calcd for | 4H), 5.84 (d, J = 6.4 Hz, 1H), 5.81 (d, J = 6.4 Hz, |
| | | | C₃₅H₄₃N₂O₉, | 1H), 5.19 (dq, J = 9.1, 6.3 Hz, 1H), |
| | | | 635.2963; | 4.85-4.73 (m, 1H), 4.28 (t, J = 9.2 Hz, 1H), 4.09 (s, |
| | | | found, | 2H), 3.90 (s, 3H), 3.59 (q, J = 7.0 Hz, 2H), |
| | | | 635.2965 | 3.12 (dd, J = 13.4, 3.5 Hz, 1H), |
| | | | | 2.26-2.12 (m, 2H), 2.02 (dt, J = 9.7, 5.0 Hz, 2H), |
| | | | | 1.69 (s, 1H), 1.64-1.54 (m, 1H), 1.42 (dq, J = 11.6, |
| | | | | 4.3 Hz, 2H), 1.33 (d, J = 6.3 Hz, 3H), |
| | | | | 1.22 (t, J = 7.0 Hz, 3H), 1.07-0.94 (m, 1H) |
| | | | | ¹³C NMR (101 MHz, CDCl₃) δ 172.52, |
| | | | | 170.05, 162.98, 160.20, 159.37, 145.86, |
| | | | | 143.88, 142.40, 140.50, 129.68, 129.04, |
| | | | | 128.24, 125.83, 121.08, 115.38, 109.68, |
| | | | | 89.57, 81.98, 75.16, 67.80, 67.19, 56.22, |
| | | | | 51.71, 45.41, 38.64, 27.12, 26.15, 24.36, |
| | | | | 21.91, 18.73, 15.02 |
| 64 | — | — | HRMS-ESI | ¹H NMR (600 MHz, CDCl₃) δ 8.71 (s, 1H), |
| | | | (m/z) | 8.34 (d, J = 5.5 Hz, 1H), 7.33-7.27 (m, 2H), |
| | | | [M + H]⁺ | 7.24 (t, J = 7.5 Hz, 2H), 7.15 (t, J = 7.4 Hz, |
| | | | calcd for | 1H), 7.12 (d, J = 7.2 Hz, 2H), 7.00 (d, J = 5.5 Hz, |
| | | | C₃₂H₃₇N₂O₇, | 1H), 6.99-6.93 (m, 3H), 5.19 (dq, J = 9.1, |
| | | | 561.2595; | 6.3 Hz, 1H), 4.76 (ddd, J = 8.6, 5.3, 3.5 Hz, |
| | | | found, | 1H), 4.26 (t, J = 9.2 Hz, 1H), 3.90 (s, 3H), |
| | | | 561.2601 | 3.11 (dd, J = 13.5, 3.6 Hz, 1H), 2.39 (s, 3H), |
| | | | | 2.20 (dd, J = 13.5, 11.2 Hz, 1H), |
| | | | | 2.17-2.12 (m, 1H), 2.00 (dd, J = 9.4, 5.4 Hz, 2H), |
| | | | | 1.66-1.57 (m, 1H), 1.48-1.39 (m, 2H), 1.31 (d, J = 6.3 Hz, |
| | | | | 5H), 1.04-0.95 (m, 1H) |
| | | | | ¹³C NMR (151 MHz, CDCl₃) δ 172.43, |
| | | | | 168.90, 162.48, 159.42, 159.37, 146.76, |
| | | | | 141.56, 140.51, 129.67, 129.04, 128.24, |
| | | | | 125.83, 121.06, 115.38, 109.75, 82.00, 75.15, |
| | | | | 56.29, 51.50, 45.39, 38.68, 27.17, 26.19, |
| | | | | 24.41, 21.82, 20.76, 18.72 |
| 65 | — | — | HRMS-ESI | ¹H NMR (500 MHz, CDCl₃) δ 8.86 (d, J = 7.3 Hz, |
| | | | (m/z) | 1H), 8.38 (d, J = 5.4 Hz, 1H), 7.31 (t, J = 7.9 Hz, |
| | | | [M + H]⁺ | 2H), 7.04 (d, J = 8.5 Hz, 2H), 7.01 (d, |
| | | | calcd for | J = 5.5 Hz, 1H), 6.97 (t, J = 7.3 Hz, 1H), |
| | | | C₃₂H₃₇N₂O₉, | 6.92 (d, J = 8.5 Hz, 2H), 6.78 (d, J = 8.5 Hz, 2H), |
| | | | 593.2494; | 5.40 (dq, J = 12.5, 6.3 Hz, 1H), 4.94 (dd, J = 8.0, |
| | | | found, | 3.2 Hz, 1H), 4.10 (t, J = 8.4 Hz, 1H), |
| | | | 593.2506 | 3.91 (s, 3H), 3.87 (dd, J = 10.4, 3.8 Hz, 1H), |
| | | | | 3.79 (d, J = 10.4 Hz, 1H), 3.76 (s, 3H), 3.12 (td, J = 13.5, |
| | | | | 12.6, 4.2 Hz, 2H), 2.80 (t, J = 11.0 Hz, |
| | | | | 1H), 2.38 (s, 3H), 2.28 (s, 1H), |
| | | | | 2.20-2.07 (m, 1H), 2.02 (t, J = 13.1 Hz, 1H), |
| | | | | 1.49-1.38 (m, 1H), 1.32 (d, J = 6.3 Hz, 3H) |
| | | | | ¹³C NMR (126 MHz, CDCl₃) δ 169.27, |
| | | | | 168.84, 162.87, 159.36, 159.22, 157.84, |
| | | | | 146.84, 141.39, 137.50, 132.23, 129.99, |
| | | | | 129.67, 121.11, 115.35, 113.77, 109.80, |
| | | | | 82.83, 74.22, 68.54, 68.22, 56.27, 55.18, |
| | | | | 53.73, 38.99, 37.64, 32.28, 20.72, 18.95 |
| 66 | — | — | HRMS-ESI | ¹H NMR (500 MHz, CDCl₃) δ 8.70 (d, J = 8.0 Hz, |
| | | | (m/z) | 1H), 8.32 (d, J = 5.4 Hz, 1H), 7.31 (t, J = 7.7 Hz, |
| | | | [M + H]⁺ | 2H), 7.03 (d, J = 8.4 Hz, 2H), |
| | | | calcd for | 7.01-6.90 (m, 4H), 6.78 (d, J = 8.2 Hz, 2H), |
| | | | C₃₃H₃₉N₂O₁₀, | 5.81-5.69 (m, 2H), 5.41 (dt, J = 12.6, 6.4 Hz, 1H), |
| | | | 623.2599; | 4.98 (dd, J = 7.9, 3.3 Hz, 1H), 4.11 (t, J = 8.4 Hz, |
| | | | found, | 1H), 3.91 (s, 3H), 3.91-3.87 (m, 1H), |
| | | | 623.2605 | 3.83 (d, J = 10.4 Hz, 1H), 3.76 (s, 3H), |
| | | | | 3.19-3.07 (m, 2H), 2.80 (t, J = 11.5 Hz, 1H), |
| | | | | 2.28 (s, 1H), 2.13 (dd, J = 13.2, 11.0 Hz, 1H), |
| | | | | 2.06 (s, 3H), 2.05-1.96 (m, 1H), 1.43 (dd, J = 11.6, |
| | | | | 3.6 Hz, 1H), 1.33 (d, J = 6.3 Hz, 3H) |
| | | | | ¹³C NMR (126 MHz, CDCl₃) δ 170.26, |
| | | | | 169.40, 163.41, 160.22, 159.22, 157.84, |
| | | | | 145.88, 143.93, 142.43, 132.23, 129.98, |
| | | | | 129.68, 121.13, 115.36, 113.77, 109.61, |
| | | | | 89.54, 82.84, 74.23, 68.53, 68.16, 56.17, |
| | | | | 55.18, 53.94, 39.00, 37.64, 32.27, 20.87, |
| | | | | 18.98 |
| 67 | — | — | HRMS-ESI | ¹H NMR (500 MHz, CDCl₃) δ 8.75 (d, J = 8.0 Hz, |
| | | | (m/z) | 1H), 8.31 (d, J = 5.4 Hz, 1H), 7.31 (tt, J = 7.3, |
| | | | [M + H]⁺ | 2.2 Hz, 2H), 7.08-7.00 (m, 2H), |
| | | | calcd for | 6.99-6.95 (m, 1H), 6.95-6.91 (m, 3H), |
| | | | C₃₅H₄₃O₁₀N₂, | 6.80-6.76 (m, 2H), 5.83-5.71 (m, 2H), 5.41 (dq, J = 9.2, |
| | | | 651.2912; | 6.2 Hz, 1H), 5.01-4.94 (m, 1H), |
| | | | found, | 4.11 (dd, J = 8.9, 7.9 Hz, 1H), 3.91-3.87 (m, 4H), |
| | | | 651.2924 | 3.83 (dd, J = 10.5, 1.1 Hz, 1H), 3.76 (s, 3H), |
| | | | | 3.23-3.07 (m, 2H), 2.89-2.75 (m, 1H), |
| | | | | 2.54 (hept, J = 7.0 Hz, 1H), 2.28 (dd, J = 10.5, 7.4 Hz, |
| | | | | 1H), 2.14 (dd, J = 13.6, 10.7 Hz, 1H), |
| | | | | 2.09-1.97 (m, 1H), 1.48-1.39 (m, 1H), |
| | | | | 1.33 (d, J = 6.3 Hz, 3H), 1.14 (dd, J = 7.0, 0.6 Hz, |
| | | | | 6H) |
| | | | | ¹³C NMR (126 MHz, CDCl₃) δ 76.21, 169.40, |
| | | | | 163.39, 160.24, 159.23, 157.84, 145.74, |
| | | | | 144.19, 142.08, 132.23, 129.99, 129.68, |
| | | | | 121.12, 115.36, 113.77, 109.56, 89.95, 82.85, |
| | | | | 74.23, 68.53, 68.17, 56.12, 55.18, 53.93, |
| | | | | 39.00, 37.64, 33.86, 32.28, 18.98, 18.68 |
| 68 | — | — | HRMS-ESI | ¹H NMR (500 MHz, CDCl₃) δ 8.81 (d, J = 6.9 Hz, |
| | | | (m/z) | 1H), 8.37 (d, J = 5.3 Hz, 1H), 7.30 (t, J = 7.7 Hz, |
| | | | [M + H]⁺ | 2H), 7.04 (d, J = 8.3 Hz, 2H), |
| | | | calcd for | 7.01-6.95 (m, 2H), 6.92 (d, J = 8.0 Hz, 2H), |
| | | | C₃₄H₄₁N₂O₉, | 6.78 (d, J = 8.2 Hz, 2H), 5.40 (p, J = 6.3 Hz, 1H), |
| | | | 621.2807; | 5.04-4.88 (m, 1H), 4.10 (t, J = 8.3 Hz, 1H), |
| | | | found, | 3.89 (s, 3H), 3.88-3.84 (m, 1H), 3.80 (s, |
| | | | 621.2818 | 1H), 3.76 (s, 3H), 3.18-3.07 (m, 2H), |
| | | | | 2.93 (p, J = 6.9 Hz, 1H), 2.80 (t, J = 11.4 Hz, 1H), |
| | | | | 2.28 (s, 1H), 2.20-2.09 (m, 1H), 2.02 (t, J = 12.9 Hz, |
| | | | | 1H), 1.43 (dd, J = 11.6, 3.9 Hz, 1H), |
| | | | | 1.35 (d, J = 7.0 Hz, 6H), 1.31 (d, J = 6.3 Hz, |
| | | | | 3H) |
| | | | | ¹³C NMR (126 MHz, CDCl₃) δ 174.61, |
| | | | | 169.36, 162.84, 159.34, 159.23, 157.84, |
| | | | | 146.74, 141.73, 137.69, 132.24, 129.99, |
| | | | | 129.67, 121.10, 115.36, 113.77, 109.64, |
| | | | | 82.84, 74.17, 68.53, 68.33, 56.28, 55.18, |
| | | | | 53.67, 38.99, 37.64, 34.67, 33.94, 32.29, |
| | | | | 18.96, 18.83 |
| 69 | — | — | HRMS-ESI | ¹H NMR (400 MHz, CDCl₃) δ 8.77 (d, J = 7.9 Hz, |
| | | | (m/z) | 1H), 8.31 (d, J = 5.3 Hz, 1H), |
| | | | [M]⁺ | 7.33-7.23 (m, 2H), 7.01-6.86 (m, 4H), 5.81-5.72 (m, |
| | | | calcd for | 2H), 5.36 (dq, J = 9.1, 6.3 Hz, 1H), 5.00 (ddd, |
| | | | C₂₇H₃₄N₂O₉, | J = 8.0, 3.5, 1.4 Hz, 1H), 4.12-3.92 (m, 3H), |
| | | | 530.2264; | 3.91 (s, 3H), 3.73 (td, J = 11.7, 2.4 Hz, 1H), |
| | | | found, | 3.48 (dt, J = 12.0, 3.6 Hz, 1H), 2.08 (s, 3H), |
| | | | 530.2264 | 2.01 (ddt, J = 14.2, 9.1, 3.0 Hz, 1H), |
| | | | | 1.93-1.81 (m, 1H), 1.76-1.57 (m, 1H), |
| | | | | 1.39 (dddd, J = 18.0, 9.3, 4.6, 2.7 Hz, 1H), 1.32 (d, |
| | | | | J = 6.3 Hz, 3H), 1.23-1.09 (m, 1H), 0.90 (t, |
| | | | | J = 7.4 Hz, 3H) |
| | | | | ¹³C NMR (126 MHz, CDCl₃) δ 170.25, |
| | | | | 169.43, 163.42, 160.23, 159.32, 145.88, |
| | | | | 143.92, 142.40, 129.59, 120.93, 115.20, |
| | | | | 109.64, 89.53, 82.88, 74.53, 68.75, 68.10, |
| | | | | 56.18, 54.02, 38.60, 32.40, 25.14, 20.88, |
| | | | | 18.96, 12.72 |
| 70 | — | — | HRMS-ESI | ¹H NMR (400 MHz, CDCl₃) δ 8.74 (d, J = 7.9 Hz, |
| | | | (m/z) | 1H), 8.30 (d, J = 5.3 Hz, 1H), 6.95 (d, J = 5.4 Hz, |
| | | | [M]⁺ | 1H), 5.80-5.70 (m, 2H), 5.12 (dq, J = 9.0, |
| | | | calcd for | 6.3 Hz, 1H), 4.94 (ddd, J = 7.9, 3.5, 1.5 Hz, |
| | | | C₂₅H₃₈N₂O₉, | 1H), 4.00-3.88 (m, 2H), 3.91 (s, 3H), |
| | | | 510.2577; | 3.71 (ddd, J = 11.8, 10.7, 2.5 Hz, 1H), |
| | | | found, | 3.46-3.38 (m, 1H), 3.37 (dd, J = 8.4, 6.3 Hz, 1H), |
| | | | 510.2582 | 3.22 (dd, J = 8.4, 6.5 Hz, 1H), 2.90 (dd, J = 9.0, |
| | | | | 7.3 Hz, 1H), 2.07 (s, 3H), 1.92-1.67 (m, |
| | | | | 3H), 1.68-1.57 (m, 1H), 1.36 (d, J = 6.3 Hz, |
| | | | | 3H), 1.33-1.11 (m, 2H), 1.00-0.87 (m, 9H) |
| | | | | ¹³C NMR (126 MHz, CDCl₃) δ 170.25, |
| | | | | 169.44, 163.40, 160.20, 145.86, 143.87, |
| | | | | 142.48, 109.58, 89.56, 85.70, 80.53, 75.07, |
| | | | | 69.34, 68.62, 56.16, 54.08, 38.67, 33.02, |
| | | | | 29.14, 25.04, 20.88, 19.54, 19.48, 19.07, |
| | | | | 12.83 |
| 71 | — | — | HRMS-ESI | ¹H NMR (500 MHz, CDCl₃) δ 8.86 (d, J = 7.5 Hz, |
| | | | (m/z) | 1H), 8.38 (d, J = 5.4 Hz, 1H), |
| | | | [M + H]⁺ | 7.34-7.28 (m, 2H), 7.10-7.05 (m, 2H), 7.01 (d, J = 5.5 Hz, |
| | | | calcd for | 1H), 7.00-6.96 (m, 1H), 6.96-6.87 (m, |
| | | | C₃₁H₃₄FN₂O₈, | 4H), 5.39 (dq, J = 9.0, 6.3 Hz, 1H), 4.95 (dd, J = 8.2, |
| | | | 581.2294; | 2.8 Hz, 1H), 4.11 (dd, J = 9.0, 7.5 Hz, |
| | | | found, | 1H), 3.91 (s, 3H), 3.88 (dd, J = 10.5, 3.9 Hz, |
| | | | 581.2305 | 1H), 3.82 (d, J = 10.2 Hz, 1H), 3.18 (dt, J = 12.0, |
| | | | | 3.7 Hz, 1H), 3.10 (dd, J = 13.7, 5.3 Hz, |
| | | | | 1H), 2.92-2.83 (m, 1H), 2.38 (s, 3H), |
| | | | | 2.36-2.28 (m, 1H), 2.20 (dd, J = 13.6, 10.1 Hz, |
| | | | | 1H), 2.02 (td, J = 11.4, 3.1 Hz, 1H), |
| | | | | 1.48-1.40 (m, 1H), 1.31 (d, J = 6.3 Hz, 3H) |
| | | | | ¹³C NMR (126 MHz, CDCl₃) δ 169.31, |
| | | | | 168.83, 162.88, 161.32 (d, J = 243.7 Hz), |
| | | | | 159.38, 159.09, 146.86, 141.36, 137.52, |
| | | | | 136.03, 130.35 (d, J = 7.7 Hz), 129.71, |
| | | | | 121.21, 115.26, 115.10 (d, J = 21.1 Hz), |
| | | | | 109.83, 82.60, 74.16, 68.42, 56.28, 53.72, |
| | | | | 38.76, 37.63, 32.42, 31.59, 20.72, 18.88 |
| 72 | — | — | HRMS-ESI | ¹H NMR (500 MHz, CDCl₃) δ 8.70 (d, J = 8.0 Hz, |
| | | | (m/z) | 1H), 8.32 (d, J = 5.4 Hz, 1H), |
| | | | [M + H]⁺ | 7.36-7.28 (m, 2H), 7.11-7.04 (m, 2H), 7.00-6.95 (m, |
| | | | calcd for | 2H), 6.95-6.88 (m, 4H), 5.78-5.72 (m, |
| | | | C₃₂H₃₆FN₂O₉, | 2H), 5.40 (dq, J = 9.0, 6.3 Hz, 1H), 4.98 (ddd, |
| | | | 611.2399; | J = 8.0, 3.6, 1.2 Hz, 1H), 4.12 (dd, J = 9.0, 7.6 Hz, |
| | | | found, | 1H), 3.92 (s, 3H), 3.91-3.88 (m, 1H), |
| | | | 611.2407 | 3.86 (dd, J = 10.5, 1.2 Hz, 1H), 3.19 (dt, J = 11.9, |
| | | | | 3.7 Hz, 1H), 3.11 (dd, J = 13.7, 5.3 Hz, |
| | | | | 1H), 2.87 (td, J = 11.7, 2.2 Hz, 1H), 2.31 (dtd, |
| | | | | J = 9.9, 7.7, 6.5, 2.8 Hz, 1H), 2.20 (dd, J = 13.7, |
| | | | | 10.1 Hz, 1H), 2.06 (s, 3H), |
| | | | | 2.06-1.99 (m, 1H), 1.48-1.40 (m, 1H), 1.33 (d, J = 6.3 Hz, |
| | | | | 3H) |
| | | | | ¹³C NMR (126 MHz, CDCl₃) δ 170.26, |
| | | | | 169.45, 163.41, 161.32 (d, J = 243.9 Hz), |
| | | | | 160.24, 159.09, 145.89, 143.95, 142.39, |
| | | | | 136.02 (d, J = 3.1 Hz), 130.35 (d, J = 7.7 Hz), |
| | | | | 129.72, 121.22, 115.10 (d, J = 21.0 Hz), |
| | | | | 109.64, 89.53, 82.62, 74.17, 68.41, 56.18, |
| | | | | 53.93, 38.77, 37.63, 32.42, 20.87, 18.91 |
| 73 | — | — | HRMS-ESI | ¹H NMR (500 MHz, CDCl₃) δ 8.76 (d, J = 8.0 Hz, |
| | | | (m/z) | 1H), 8.32 (d, J = 5.4 Hz, 1H), 7.31 (tt, J = 7.5, |
| | | | [M + H]⁺ | 2.2 Hz, 2H), 7.08 (ddd, J = 8.3, 5.2, 2.5 Hz, |
| | | | calcd for | 2H), 7.01-6.96 (m, 1H), 6.96-6.88 (m, |
| | | | C₃₄H₄₀FN₂O₉, | 5H), 5.82-5.72 (m, 2H), 5.40 (dq, J = 9.0, |
| | | | 639.2712; | 6.3 Hz, 1H), 4.98 (ddd, J = 8.0, 3.7, 1.2 Hz, |
| | | | found, | 1H), 4.12 (dd, J = 9.0, 7.5 Hz, 1H), |
| | | | 639.2721 | 3.93-3.83 (m, 5H), 3.19 (dt, J = 12.0, 3.7 Hz, 1H), |
| | | | | 3.11 (dd, J = 13.6, 5.3 Hz, 1H), 2.88 (td, J = 11.7, |
| | | | | 2.2 Hz, 1H), 2.55 (hept, J = 7.0 Hz, 1H), |
| | | | | 2.32 (dtd, J = 10.0, 7.6, 6.5, 2.8 Hz, 1H), |
| | | | | 2.20 (dd, J = 14.0, 10.5 Hz, 1H), 2.03 (td, J = 11.3, |
| | | | | 5.5 Hz, 1H), 1.49-1.39 (m, 1H), 1.33 (d, J = 6.3 Hz, |
| | | | | 3H), 1.14 (d, J = 7.0 Hz, 3H), 1.14 (d, |
| | | | | J = 7.0 Hz, 3H) |
| | | | | ¹³C NMR (126 MHz, CDCl₃) δ 176.21, |
| | | | | 169.44, 163.39, 161.32 (d, J = 243.6 Hz), |
| | | | | 160.25, 159.09, 145.75, 144.21, 142.04, |
| | | | | 136.02 (d, J = 3.2 Hz), 130.35 (d, J = 7.7 Hz), |
| | | | | 129.72, 121.21, 115.26, 115.10 (d, J = 21.1 Hz), |
| | | | | 109.58, 89.94, 82.62, 74.16, 68.41, |
| | | | | 56.13, 53.92, 38.77, 37.63, 33.86, 32.42, |
| | | | | 18.91, 18.68 |
| 74 | — | — | HRMS-ESI | ¹H NMR (500 MHz, CDCl₃) δ 8.66 (d, J = 7.9 Hz, |
| | | | (m/z) | 1H), 8.30 (d, J = 5.4 Hz, 1H), |
| | | | [M]⁺ | 7.19-7.08 (m, 2H), 6.99-6.89 (m, 3H), 5.76-5.69 (m, |
| | | | calcd for | 2H), 5.21 (dq, J = 9.2, 6.3 Hz, 1H), 4.92 (dt, J = 8.0, |
| | | | C₃₀H₃₇FN₂O₉, | 2.5 Hz, 1H), 3.90 (s, 3H), 3.80 (d, J = 2.6 Hz, |
| | | | 588.2483; | 2H), 3.55-3.45 (m, 1H), 3.38 (dd, J = 9.8, |
| | | | found, | 6.9 Hz, 1H), 3.30 (dd, J = 13.4, 4.4 Hz, |
| | | | 588.2490 | 1H), 3.07 (dt, J = 12.0, 3.8 Hz, 1H), 3.01 (dd, |
| | | | | J = 9.2, 7.5 Hz, 1H), 2.75-2.64 (m, 1H), |
| | | | | 2.25-2.14 (m, 1H), 2.05 (s, 3H), 2.02 (ddd, J = 11.2, |
| | | | | 7.5, 4.8 Hz, 1H), 1.87 (ddt, J = 14.5, |
| | | | | 11.1, 3.3 Hz, 1H), 1.40 (d, J = 6.3 Hz, 3H), |
| | | | | 1.30 (dddd, J = 15.3, 7.0, 4.3, 2.4 Hz, 1H), |
| | | | | 1.16-1.04 (m, 1H), 0.62-0.50 (m, 2H), |
| | | | | 0.27-0.16 (m, 2H) |
| | | | | ¹⁹F NMR (471 MHz, CDCl₃) δ −117.63 (td, J = 9.1, |
| | | | | 4.6 Hz) |
| 75 | — | — | HRMS-ESI | ¹H NMR (500 MHz, CDCl₃) δ 8.83 (d, J = 7.7 Hz, |
| | | | (m/z) | 1H), 8.37 (d, J = 5.4 Hz, 1H), |
| | | | [M + H]⁺ | 7.15-7.07 (m, 2H), 7.01 (d, J = 5.5 Hz, 1H), |
| | | | calcd for | 6.99-6.89 (m, 2H), 5.33 (dq, J = 10.3, 7.2, 6.8 Hz, 1H), |
| | | | C₂₉H₃₆FN₂O₉, | 5.01-4.89 (m, 1H), 4.79 (dd, J = 9.4, 7.3 Hz, |
| | | | 575.2399; | 1H), 3.91 (s, 3H), 3.89-3.78 (m, 2H), |
| | | | found, | 3.14 (dt, J = 11.9, 3.8 Hz, 1H), 2.91-2.79 (m, |
| | | | 575.2405 | 2H), 2.53 (h, J = 7.0 Hz, 1H), 2.38 (s, 3H), |
| | | | | 2.29-2.19 (m, 1H), 2.15 (dd, J = 12.9, 10.4 Hz, |
| | | | | 1H), 1.94 (ddt, J = 13.9, 6.6, 3.3 Hz, 1H), |
| | | | | 1.36 (ddt, J = 14.5, 6.9, 2.5 Hz, 1H), 1.26 (d, J = 6.3 Hz, |
| | | | | 3H), 1.19 (d, J = 3.3 Hz, 3H), |
| | | | | 1.17 (d, J = 3.4 Hz, 3H) |
| | | | | ¹³C NMR (126 MHz, CDCl₃) δ 176.53, |
| | | | | 169.42, 168.84, 162.86, 161.41 (d, J = 244.2 Hz), |
| | | | | 159.39, 146.83, 141.33, 137.54, |
| | | | | 135.38 (d, J = 3.2 Hz), 130.38 (d, J = 7.7 Hz), |
| | | | | 115.25 (d, J = 21.1 Hz), 109.84, 72.75, 68.93, 68.66, |
| | | | | 56.29, 53.74, 36.91, 36.82, 34.11, 31.70, |
| | | | | 20.72, 19.09, 18.84, 18.50 |
| 76 | — | — | HRMS-ESI | ¹H NMR (500 MHz, CDCl₃) δ 8.66 (d, J = 8.1 Hz, |
| | | | (m/z) | 1H), 8.31 (d, J = 5.4 Hz, 1H), |
| | | | [M + H]⁺ | 7.15-7.08 (m, 2H), 6.96 (dp, J = 5.0, 3.0 Hz, 3H), |
| | | | calcd for | 5.74 (s, 2H), 5.34 (dq, J = 9.4, 6.3 Hz, 1H), |
| | | | C₃₀H₃₈FN₂O₁₀, | 4.98 (ddd, J = 8.1, 3.1, 1.9 Hz, 1H), 4.80 (dd, J = 9.4, |
| | | | 605.2505; | 7.3 Hz, 1H), 3.91 (s, 3H), 3.90-3.81 (m, |
| | | | found, | 2H), 3.15 (dt, J = 11.9, 3.7 Hz, 1H), |
| | | | 605.2516 | 2.92-2.80 (m, 2H), 2.54 (hept, J = 7.0 Hz, 1H), |
| | | | | 2.29-2.11 (m, 2H), 2.06 (s, 3H), |
| | | | | 2.00-1.91 (m, 1H), 1.36 (dtd, J = 11.0, 6.9, 2.6 Hz, 1H), |
| | | | | 1.27 (d, J = 6.3 Hz, 3H), 1.19 (d, J = 3.1 Hz, |
| | | | | 3H), 1.17 (d, J = 3.1 Hz, 3H) |
| | | | | ¹³C NMR (126 MHz, CDCl₃) δ 176.53, |
| | | | | 170.26, 169.56, 163.39, 161.41 (d, J = 244.1 Hz), |
| | | | | 160.25, 145.87, 143.99, 142.36, |
| | | | | 135.38 (d, J = 3.2 Hz), 130.38 (d, J = 7.7 Hz), |
| | | | | 115.25 (d, J = 21.1 Hz), 109.64, 89.54, 72.77, 68.87, |
| | | | | 68.66, 56.19, 53.96, 36.91, 36.83, 34.12, |
| | | | | 31.70, 29.28, 20.87, 19.09, 18.85, 18.52. |
| 77 | — | — | HRMS-ESI | ¹H NMR (500 MHz, CDCl₃) δ 8.81 (d, J = 8.1 Hz, |
| | | | (m/z) | 1H), 8.35 (d, J = 5.4 Hz, 1H), |
| | | | [M]⁺ | 7.19-7.07 (m, 2H), 7.03-6.90 (m, 3H), 5.20 (dq, J = 9.2, |
| | | | calcd for | 6.3 Hz, 1H), 4.88 (ddd, J = 8.2, 3.2, 1.5 Hz, |
| | | | C₂₉H₃₅FN₂O₈, | 1H), 3.89 (s, 3H), 3.83-3.73 (m, 2H), |
| | | | 558.2377; | 3.49 (dd, J = 9.8, 6.9 Hz, 1H), 3.37 (dd, J = 9.8, |
| | | | found, | 6.9 Hz, 1H), 3.29 (dd, J = 13.4, 4.4 Hz, |
| | | | 558.2383 | 1H), 3.06 (dt, J = 12.0, 3.8 Hz, 1H), 3.00 (dd, |
| | | | | J = 9.2, 7.5 Hz, 1H), 2.77-2.65 (m, 1H), |
| | | | | 2.37 (s, 3H), 2.25-2.13 (m, 1H), |
| | | | | 2.09-1.98 (m, 1H), 1.86 (ddt, J = 14.5, 11.1, 3.4 Hz, |
| | | | | 1H), 1.39 (d, J = 6.3 Hz, 3H), 1.36-1.21 (m, |
| | | | | 1H), 1.17-0.99 (m, 1H), 0.62-0.50 (m, |
| | | | | 2H), 0.27-0.15 (m, 2H) |
| | | | | ¹⁹F NMR (471 MHz, CDCl₃) δ −117.60 (ddd, |
| | | | | J = 14.0, 9.0, 5.4 Hz) |
| 78 | — | — | HRMS-ESI | ¹H NMR (500 MHz, CDCl₃) δ 8.66 (d, J = 7.9 Hz, |
| | | | (m/z) | 1H), 8.30 (d, J = 5.4 Hz, 1H), |
| | | | [M]⁺ | 7.19-7.08 (m, 2H), 7.01-6.88 (m, 3H), 5.81 (s, 2H), |
| | | | calcd for | 5.21 (dq, J = 9.1, 6.3 Hz, 1H), 4.90 (ddd, J = 8.1, |
| | | | C₃₂H₄₁FN₂O₁₀, | 3.2, 1.9 Hz, 1H), 4.08 (s, 2H), 3.90 (s, |
| | | | 632.2745; | 3H), 3.84-3.75 (m, 2H), 3.57 (q, J = 7.0 Hz, |
| | | | found, | 2H), 3.49 (dd, J = 9.8, 6.9 Hz, 1H), 3.38 (dd, J = 9.8, |
| | | | 632.2752 | 6.9 Hz, 1H), 3.30 (dd, J = 13.4, 4.3 Hz, |
| | | | | 1H), 3.06 (dt, J = 12.0, 3.8 Hz, 1H), 3.01 (dd, |
| | | | | J = 9.1, 7.6 Hz, 1H), 2.70 (td, J = 11.5, 11.1, |
| | | | | 2.3 Hz, 1H), 2.25-2.16 (m, 1H), 2.03 (tdq, J = 11.2, |
| | | | | 7.3, 3.7, 3.3 Hz, 1H), 1.87 (ddt, J = 14.5, |
| | | | | 11.1, 3.3 Hz, 1H), 1.40 (d, J = 6.2 Hz, |
| | | | | 3H), 1.37-1.26 (m, 1H), 1.22 (t, J = 7.0 Hz, |
| | | | | 3H), 1.16-1.04 (m, 1H), 0.62-0.52 (m, |
| | | | | 2H), 0.27-0.18 (m, 2H) |
| | | | | ¹⁹F NMR (471 MHz, CDCl₃) δ −117.61 (ddd, |
| | | | | J = 14.1, 9.3, 5.4 Hz) |
| 79 | — | — | HRMS-ESI | ¹H NMR (400 MHz, CDCl₃) δ 8.73 (d, J = 8.0 Hz, |
| | | | (m/z) | 1H), 8.30 (d, J = 5.4 Hz, 1H), 6.96 (d, J = 5.4 Hz, |
| | | | [M]⁺ | 1H), 5.80-5.71 (m, 2H), 5.26 (dq, J = 9.2, |
| | | | calcd for | 6.3 Hz, 1H), 4.99 (ddd, J = 8.1, 3.0, 2.1 Hz, |
| | | | C₂₅H₃₄N₂O₁₀, | 1H), 4.74 (dd, J = 9.2, 7.7 Hz, 1H), |
| | | | 522.2213; | 4.00-3.94 (m, 2H), 3.91 (s, 3H), 3.71 (ddd, J = 11.7, |
| | | | found, | 10.4, 2.6 Hz, 1H), 3.44 (ddd, J = 11.9, |
| | | | 522.2221 | 4.6, 3.3 Hz, 1H), 2.07 (s, 3H), 1.90 (ddt, J = 14.1, |
| | | | | 10.4, 3.4 Hz, 1H), 1.84-1.75 (m, 1H), |
| | | | | 1.65 (tt, J = 8.0, 4.6 Hz, 1H), 1.53-1.39 (m, |
| | | | | 1H), 1.38-1.31 (m, 1H), 1.28 (d, J = 6.4 Hz, |
| | | | | 3H), 1.17 (dp, J = 14.3, 7.3 Hz, 1H), |
| | | | | 1.08-0.97 (m, 2H), 0.90 (td, J = 7.5, 3.8 Hz, 5H) |
| | | | | ¹³C NMR (101 MHz, CDCl₃) δ 174.38, |
| | | | | 170.25, 169.52, 163.39, 160.24, 145.84, |
| | | | | 143.97, 142.39, 109.63, 89.55, 76.86, 73.29, |
| | | | | 69.01, 68.72, 56.18, 54.05, 36.90, 31.42, |
| | | | | 24.29, 20.87, 18.53, 12.89, 11.93, 8.55, 8.30 |
| 80 | — | — | HRMS-ESI | ¹H NMR (400 MHz, CDCl₃) δ 8.92 (d, J = 8.5 Hz, |
| | | | (m/z) | 1H), 8.38 (dd, J = 5.4, 2.5 Hz, 1H), |
| | | | [M]⁺ | 8.11-8.03 (m, 2H), 7.65-7.55 (m, 1H), |
| | | | calcd for | 7.53-7.43 (m, 2H), 7.02 (dd, J = 5.5, 1.6 Hz, 1H), |
| | | | C₂₇H₃₂N₂O₉, | 5.43 (dq, J = 9.3, 6.3 Hz, 1H), 5.06-4.95 (m, 2H), |
| | | | 528.2108; | 4.03-3.94 (m, 2H), 3.91 (s, 3H), |
| | | | found, | 3.82-3.71 (m, 1H), 3.49 (ddd, J = 11.9, 4.8, 3.0 Hz, 1H), |
| | | | 528.2106 | 2.40 (s, 3H), 1.97 (dddd, J = 17.6, 10.2, 6.9, |
| | | | | 3.7 Hz, 2H), 1.59-1.36 (m, 2H), 1.32 (d, J = 6.4 Hz, |
| | | | | 3H), 1.24-1.12 (m, 1H), 0.89 (t, J = 7.4 Hz, |
| | | | | 3H) |
| | | | | ¹³C NMR (101 MHz, CDCl₃) δ 169.46, |
| | | | | 168.85, 165.96, 162.87, 159.39, 146.83, |
| | | | | 141.35, 137.53, 133.32, 129.73, 129.65, |
| | | | | 128.56, 109.85, 77.54, 73.30, 69.13, 68.95, |
| | | | | 56.29, 53.87, 53.80, 37.14, 31.61, 29.28, |
| | | | | 24.53, 20.73, 18.59, 12.04 |
| 81 | — | — | HRMS-ESI | ¹H NMR (400 MHz, CDCl₃) δ 12.02 (s, 1H), |
| | | | (m/z) | 8.84 (d, J = 8.4 Hz, 1H), 8.04 (d, J = 5.2 Hz, |
| | | | [M + H]⁺ | 1H), 7.38-7.27 (m, 10H), 6.88 (d, J = 5.2 Hz, |
| | | | calcd for | 1H), 5.43-5.26 (m, 1H), 5.04 (d, J = 11.2 Hz, |
| | | | C₃₀H₃₅N₂O₈, | 1H), 4.94 (d, J = 11.4 Hz, 1H), |
| | | | 551.2388; | 4.90 (dd, J = 8.3, 2.1 Hz, 1H), 4.59 (d, J = 11.2 Hz, |
| | | | found, | 1H), 4.55 (d, J = 11.4 Hz, 1H), 3.95 (s, 3H), |
| | | | 551.2378 | 3.93-3.83 (m, 2H), 3.63 (q, J = 8.1, 6.9 Hz, |
| | | | | 2H), 3.40 (dt, J = 12.2, 3.0 Hz, 1H), 3.32 (t, J = 8.9 Hz, |
| | | | | 1H), 2.10 (t, J = 12.8 Hz, 1H), |
| | | | | 1.66-1.57 (m, 1H), 1.35 (d, J = 6.3 Hz, 3H) |
| | | | | ¹³C NMR (101 MHz, CDCl₃) δ 168.99, |
| | | | | 168.73, 155.27, 148.70, 140.67, 138.75, |
| | | | | 138.08, 130.42, 128.40, 128.34, 128.12, |
| | | | | 128.02, 127.79, 127.59, 109.49, 84.54, 77.82, |
| | | | | 75.25, 75.20, 73.48, 66.85, 66.75, 56.07, |
| | | | | 53.69, 38.61, 34.22, 19.01. |
| 82 | — | — | HRMS-ESI | ¹H NMR (400 MHz, CDCl₃) δ 12.02 (s, 1H), |
| | | | (m/z) | 8.86 (d, J = 8.3 Hz, 1H), 8.02 (d, J = 5.2 Hz, |
| | | | [M + Na]⁺ | 1H), 7.41-7.27 (m, 5H), 6.87 (d, J = 5.2 Hz, |
| | | | calcd for | 1H), 5.35-5.17 (m, 1H), 5.02 (d, J = 11.3 Hz, |
| | | | C₂₇H₃₆N₂NaO₈, | 1H), 4.96-4.85 (m, 1H), 4.56 (d, J = 11.3 Hz, |
| | | | 539.2364; | 1H), 4.01-3.83 (m, 6H), 3.77 (td, J = 12.4, |
| | | | found, | 1.8 Hz, 1H), 3.52-3.36 (m, 3H), |
| | | | 539.2404 | 3.24 (t, J = 8.9 Hz, 1H), 2.15-2.02 (m, 1H), |
| | | | | 1.57-1.45 (m, 3H), 1.38-1.25 (m, 5H), 0.88 (t, J = 7.4 Hz, |
| | | | | 3H) |
| | | | | ¹³C NMR (101 MHz, CDCl₃) δ 169.01, |
| | | | | 168.69, 155.28, 148.71, 140.68, 138.29, |
| | | | | 130.42, 128.37, 127.98, 127.70, 109.49, |
| | | | | 84.39, 78.23, 75.06, 73.49, 73.13, 67.16, |
| | | | | 67.09, 66.85, 56.06, 53.74, 34.31, 32.52, |
| | | | | 19.29, 18.98, 13.96. |
| 83 | — | (Neat) | — | ¹H NMR (400 MHz, CDCl₃) δ 12.02 (s, 1H), |
| | | 3379, | | 8.84 (d, J = 8.3 Hz, 1H), 8.12-7.92 (m, 1H), |
| | | 2961, | | 6.89 (d, J = 5.2 Hz, 1H), 5.51-5.25 (m, 1H), |
| | | 2935, | | 5.04-4.89 (m, 1H), 4.79 (t, J = 8.7 Hz, 1H), |
| | | 2873, | | 4.06-3.87 (m, 5H), 3.79 (t, J = 11.6 Hz, 1H), |
| | | 1738, | | 3.70-3.53 (m, 1H), 3.53-3.32 (m, 3H), |
| | | 1650, | | 2.71-2.49 (m, 1H), 2.30-2.06 (m, 1H), 1.63 (dt, |
| | | 1576, | | J = 15.6, 3.2 Hz, 1H), 1.56-1.38 (m, 2H), |
| | | 1527, | | 1.38-1.27 (m, 5H), 1.22 (t, J = 7.0 Hz, 6H), |
| | | 1481, | | 0.89 (t, J = 7.3 Hz, 3H) |
| | | 1438, | | ¹³C NMR (101 MHz, CDCl₃) δ 175.98, |
| | | 1333, | | 169.00, 168.81, 155.29, 148.72, 140.67, |
| | | 1262, | | 130.31, 109.55, 75.60, 75.56, 72.67, 72.33, |
| | | 1241, | | 67.60, 67.47, 56.06, 53.65, 34.01, 33.77, |
| | | 1184, | | 32.15, 19.23, 19.19, 18.59, 13.90 |
| | | 1141, | | |
| | | 1078, | | |
| | | 799 | | |
| 84 | — | — | HRMS-ESI | ¹H NMR (400 MHz, CDCl₃) δ 12.02 (d, J = 0.5 Hz, |
| | | | (m/z) | 1H), 8.87 (d, J = 8.3 Hz, 1H), |
| | | | [M + H]⁺ | 8.20-8.07 (m, 2H), 8.03 (d, J = 5.2 Hz, 1H), |
| | | | calcd for | 7.64-7.54 (m, 1H), 7.54-7.40 (m, 2H), 6.89 (d, J = 5.2 Hz, |
| | | | C₂₇H₃₅N₂O₉, | 1H), 5.57 (dq, J = 8.9, 6.4 Hz, 1H), |
| | | | 531.2337; | 5.15-5.04 (m, 1H), 5.01 (ddd, J = 8.4, 3.6, |
| | | | found, | 1.2 Hz, 1H), 4.08-3.96 (m, 2H), 3.95 (s, |
| | | | 531.2342 | 3H), 3.81 (td, J = 12.0, 2.1 Hz, 1H), |
| | | | | 3.57-3.47 (m, 3H), 3.43 (dt, J = 9.0, 6.6 Hz, 1H), |
| | | | | 2.26 (ddt, J = 15.2, 11.8, 3.1 Hz, 1H), |
| | | | | 1.77-1.63 (m, 1H), 1.42 (d, J = 6.4 Hz, 3H), |
| | | | | 1.36-1.01 (m, 4H), 0.67 (t, J = 7.3 Hz, 3H) |
| | | | | ¹³C NMR (101 MHz, CDCl₃) δ, 169.04, |
| | | | | 168.92, 165.56, 155.33, 148.76, 140.69, |
| | | | | 133.18, 130.37, 129.79, 129.73, 128.44, |
| | | | | 109.56, 76.53, 76.07, 73.06, 72.32, 67.47, |
| | | | | 67.30, 56.09, 53.71, 33.71, 32.02, 18.99, |
| | | | | 18.72, 13.71 |
| 85 | — | — | HRMS-ESI | ¹H NMR (400 MHz, CDCl₃) δ 12.00 (s, 1H), |
| | | | (m/z) | 8.85 (d, J = 8.3 Hz, 1H), 8.03 (d, J = 5.2 Hz, |
| | | | [M + H]⁺ | 1H), 6.88 (d, J = 5.2 Hz, 1H), 5.40 (dq, J = 9.1, |
| | | | calcd for | 6.4 Hz, 1H), 4.96 (dd, J = 8.4, 2.2 Hz, |
| | | | C₂₄H₃₅N₂O₉, | 1H), 4.81 (t, J = 8.8 Hz, 1H), 4.01-3.88 (m, |
| | | | 495.2337; | 5H), 3.77 (dd, J = 12.2, 10.0 Hz, 1H), |
| | | | found, | 3.64 (dt, J = 9.1, 6.7 Hz, 1H), 3.52-3.43 (m, 2H), |
| | | | 495.2340 | 3.43-3.35 (m, 1H), 2.14 (dd, J = 14.8, 12.4 Hz, |
| | | | | 1H), 1.65 (tq, J = 9.7, 5.0 Hz, 2H), |
| | | | | 1.55-1.42 (m, 2H), 1.40-1.29 (m, 5H), 1.06 (dt, J = 7.5, |
| | | | | 3.5 Hz, 2H), 0.95-0.85 (m, 5H) |
| | | | | ¹³C NMR (101 MHz, CDCl₃) δ 174.09, |
| | | | | 169.03, 168.83, 155.32, 148.75, 140.69, |
| | | | | 130.38, 109.53, 76.10, 75.76, 73.01, 72.31, |
| | | | | 67.49, 67.27, 56.09, 53.69, 33.72, 32.24, |
| | | | | 19.26, 18.71, 13.97, 12.81, 8.83, 8.58. |
| 86 | — | — | HRMS-ESI | ¹H NMR (400 MHz, CDCl₃) δ |
| | | | (m/z) | 12.17-11.86 (m, 1H), 8.86 (d, J = 8.3 Hz, 1H), 8.02 (d, J = 5.2 Hz, |
| | | | [M + H]⁺ | 1H), 6.87 (d, J = 5.2 Hz, 1H), 5.19 (dt, |
| | | | calcd for | J = 8.9, 6.3 Hz, 1H), 4.97-4.83 (m, 1H), |
| | | | C₂₄H₃₉N₂O₈, | 3.95 (s, 3H), 3.93-3.87 (m, 2H), 3.80 (dd, J = 8.7, |
| | | | 483.2701; | 5.9 Hz, 1H), 3.78-3.71 (m, 1H), |
| | | | found, | 3.65 (dd, J = 9.2, 6.4 Hz, 1H), 3.43 (dt, J = 12.2, |
| | | | 483.2708 | 3.0 Hz, 1H), 3.36-3.29 (m, 1H), 3.16 (dd, J = 9.2, |
| | | | | 6.9 Hz, 1H), 3.12 (dd, J = 8.7, 7.2 Hz, |
| | | | | 1H), 3.04 (t, J = 8.9 Hz, 1H), 2.05 (t, J = 12.9 Hz, |
| | | | | 1H), 1.81 (dp, J = 20.1, 6.7 Hz, 2H), |
| | | | | 1.46 (dd, J = 15.6, 6.8 Hz, 1H), 1.38 (d, J = 6.3 Hz, |
| | | | | 3H), 0.97-0.90 (m, 6H), 0.90-0.85 (m, 6H) |
| | | | | ¹³C NMR (101 MHz, CDCl₃) δ 169.00, |
| | | | | 168.71, 155.27, 148.70, 140.67, 130.45, |
| | | | | 109.47, 85.58, 80.54, 80.12, 78.48, 73.66, |
| | | | | 67.26, 66.87, 56.06, 53.75, 34.28, 29.09, |
| | | | | 28.99, 19.62, 19.46, 19.39, 19.34, 19.03 |
| 87 | — | — | HRMS-ESI | ¹H NMR (400 MHz, CDCl₃) δ |
| | | | (m/z) | 12.15-11.92 (m, 1H), 8.86 (d, J = 8.2 Hz, 1H), 8.02 (d, J = 5.2 Hz, |
| | | | [M + H]⁺ | 1H), 6.88 (d, J = 5.2 Hz, 1H), |
| | | | calcd for | 5.20 (dq, J = 8.8, 6.3 Hz, 1H), 4.98-4.82 (m, 1H), |
| | | | C₂₄H₃₉N₂O₈, | 4.02-3.90 (m, 5H), 3.90-3.67 (m, 3H), |
| | | | 483.2682; | 3.49-3.38 (m, 2H), 3.33 (t, J = 7.4 Hz, 1H), |
| | | | found, | 3.14 (dd, J = 8.7, 7.1 Hz, 1H), 3.04 (t, J = 8.9 Hz, |
| | | | 483.2701 | 1H), 2.13-1.96 (m, 1H), 1.91-1.75 (m, |
| | | | | 1H), 1.62-1.40 (m, 3H), 1.38 (d, J = 6.3 Hz, |
| | | | | 3H), 1.36-1.28 (m, 2H), 0.98-0.83 (m, 9H) |
| | | | | ¹³C NMR (101 MHz, CDCl₃) δ 168.98, |
| | | | | 168.71, 155.25, 148.68, 140.66, 130.40, |
| | | | | 109.49, 85.52, 80.55, 78.01, 73.66, 72.91, |
| | | | | 67.16, 66.83, 56.04, 53.73, 34.24, 32.47, |
| | | | | 29.08, 19.57, 19.38, 19.27, 19.01, 13.99 |
| 88 | — | — | HRMS-ESI | ¹H NMR (400 MHz, CDCl₃) δ 12.12 (s, 1H), |
| | | | (m/z) | 8.67 (d, J = 7.5 Hz, 1H), 8.00 (d, J = 5.2 Hz, |
| | | | [M + H]⁺ | 1H), 7.40-7.26 (m, 10H), 6.86 (d, J = 5.2 Hz, |
| | | | calcd for | 1H), 5.10-5.01 (m, 1H), 4.98 (d, J = 10.8 Hz, |
| | | | C₃₁H₃₇N₂O₇, | 1H), 4.80-4.71 (m, 1H), |
| | | | 549.2595; | 4.70-4.55 (m, 3H), 3.94 (s, 3H), 3.48 (t, J = 8.7 Hz, |
| | | | found, | 1H), 3.40 (ddd, J = 8.4, 5.5, 3.0 Hz, 1H), |
| | | | 549.2592 | 2.23-2.12 (m, 1H), 2.12-2.01 (m, 1H), |
| | | | | 1.93-1.78 (m, 1H), 1.64-1.47 (m, 3H), |
| | | | | 1.46-1.38 (m, 4H), 1.33-1.20 (m, 1H) |
| | | | | ¹³C NMR (101 MHz, CDCl₃) δ 171.76, |
| | | | | 168.71, 155.36, 148.71, 140.58, 138.57, |
| | | | | 138.25, 130.52, 128.40, 128.36, 128.05, |
| | | | | 127.98, 127.74, 127.60, 109.46, 84.01, 82.34, |
| | | | | 75.75, 72.88, 72.70, 56.08, 51.67, 28.71, |
| | | | | 27.07, 22.38, 21.59, 18.35 |
| 89 | — | — | HRMS-ESI | ¹H NMR (400 MHz, CDCl₃) δ 12.13 (s, 1H), |
| | | | (m/z) | 8.68 (d, J = 7.5 Hz, 1H), 8.00 (d, J = 5.2 Hz, |
| | | | [M + H]⁺ | 1H), 6.87 (d, J = 5.2 Hz, 1H), 4.92 (dq, J = 9.1, |
| | | | calcd for | 6.3 Hz, 1H), 4.72 (ddd, J = 7.6, 5.6, 3.4 Hz, |
| | | | C₂₅H₄₀N₂O₇, | 1H), 3.94 (s, 3H), 3.72 (dd, J = 8.4, 6.0 Hz, |
| | | | 481.2908; | 1H), 3.32-3.16 (m, 4H), 3.12 (ddd, J = 8.4, |
| | | | found, | 6.0, 2.8 Hz, 1H), 2.25-2.14 (m, 1H), |
| | | | 481.2911 | 2.06 (ddt, J = 14.6, 7.8, 4.8 Hz, 1H), |
| | | | | 1.90-1.73 (m, 3H), 1.61 (ddt, J = 11.0, 8.3, 4.0 Hz, |
| | | | | 2H), 1.53 (ddt, J = 16.0, 8.2, 4.6 Hz, 1H), |
| | | | | 1.45-1.33 (m, 4H), 1.31-1.18 (m, 1H), |
| | | | | 0.96-0.86 (m, 12H) |
| | | | | ¹³C NMR (101 MHz, CDCl₃) δ 171.89, |
| | | | | 168.66, 155.33, 148.69, 140.55, 130.53, |
| | | | | 109.42, 84.12, 83.19, 80.62, 73.04, 56.06, |
| | | | | 51.56, 29.12, 28.97, 28.20, 27.28, 22.28, |
| | | | | 21.95, 19.63, 19.56, 19.51, 19.40, 18.17 |
| 90 | — | — | HRMS-ESI | ¹H NMR (400 MHz, CDCl₃) δ 12.11 (s, 1H), |
| | | | (m/z) | 8.70 (d, J = 7.5 Hz, 1H), 8.01 (d, J = 5.2 Hz, |
| | | | [M + H]⁺ | 1H), 7.40-7.18 (m, 2H), 7.06-6.79 (m, |
| | | | calcd for | 4H), 5.05 (dq, J = 9.3, 6.3 Hz, 1H), 4.78 (ddd, |
| | | | C₂₇H₃₇N₂O₇, | J = 8.6, 5.3, 3.5 Hz, 1H), 4.25-4.08 (m, 1H), |
| | | | 501.2595; | 3.95 (s, 3H), 3.64 (dd, J = 8.4, 6.4 Hz, 1H), |
| | | | found, | 3.49-3.38 (m, 1H), 3.34 (dd, J = 8.4, 6.5 Hz, |
| | | | 501.2624 | 1H), 2.33-2.19 (m, 1H), 2.07 (dq, J = 15.0, |
| | | | | 5.4 Hz, 1H), 2.01-1.89 (m, 1H), 1.69 (tt, J = 13.2, |
| | | | | 6.5 Hz, 2H), 1.56 (dq, J = 11.2, 6.5 Hz, |
| | | | | 3H), 1.45 (d, J = 63 Hz, 3H), 1.30-1.21 (m, |
| | | | | 1H), 0.80 (d, J = 6.7 Hz, 3H), 0.75 (d, J = 6.7 Hz, |
| | | | | 3H) |
| | | | | ¹³C NMR (101 MHz, CDCl₃) δ 171.89, |
| | | | | 168.72, 158.00, 155.35, 148.70, 140.60, |
| | | | | 130.48, 129.39, 120.65, 115.69, 109.47, |
| | | | | 83.71, 80.78, 80.71, 72.68, 56.08, 51.34, |
| | | | | 28.94, 28.08, 26.89, 21.77, 21.64, 19.39, |
| | | | | 19.31, 18.20 |
| 91 | — | — | HRMS-ESI | ¹H NMR (400 MHz, CDCl₃) δ |
| | | | (m/z) | 12.14-12.00 (m, 1H), 8.70 (d, J = 7.5 Hz, 1H), 8.01 (d, J = 5.2 Hz, |
| | | | [M + H]⁺ | 1H), 7.35-7.18 (m, 2H), 7.03 (dt, J = 9.3, |
| | | | calcd for | 1.8 Hz, 2H), 6.99-6.91 (m, 1H), 6.88 (d, |
| | | | C₂₄H₃₁N₂O₇, | J = 5.2 Hz, 1H), 5.17 (dq, J = 9.3, 6.3 Hz, |
| | | | 459.2126; | 1H), 4.78 (ddd, J = 7.6, 5.4, 3.5 Hz, 1H), |
| | | | found, | 4.27 (t, J = 8.9 Hz, 1H), 3.94 (s, 3H), 3.31 (s, 3H), |
| | | | 459.2121 | 3.23 (ddd, J = 8.7, 5.9, 3.1 Hz, 1H), 2.21 (ddd, |
| | | | | J = 15.1, 7.7, 3.8 Hz, 1H), 2.11 (ddt, J = 14.8, |
| | | | | 8.2, 4.9 Hz, 1H), 1.98-1.83 (m, 1H), 1.69 (tt, |
| | | | | J = 14.7, 6.1 Hz, 2H), 1.54 (ddq, J = 16.6, 7.7, |
| | | | | 4.3, 3.9 Hz, 2H), 1.36 (d, J = 6.3 Hz, 3H), |
| | | | | 1.34-1.25 (m, 1H) |
| | | | | ¹³C NMR (101 MHz, CDCl₃) δ 171.81, |
| | | | | 168.71, 159.56, 155.36, 148.71, 140.60, |
| | | | | 130.46, 129.37, 121.29, 116.17, 109.50, |
| | | | | 83.58, 82.92, 72.61, 58.56, 56.08, 51.61, |
| | | | | 28.05, 27.22, 22.28, 21.76, 18.35 |
| 92 | — | (Neat) | HRMS-ESI | ¹H NMR (400 MHz, CDCl₃) δ 12.12 (d, J = 0.6 Hz, |
| | | 3360, | (m/z) | 1H), 8.67 (d, J = 7.5 Hz, 1H), 8.00 (d, |
| | | 2954, | [M + Na]⁺ | J = 5.2 Hz, 1H), 6.92-6.83 (m, 1H), |
| | | 1723, | calcd for | 4.93 (dq, J = 9.3, 6.3 Hz, 1H), 4.73 (ddd, J = 7.5, |
| | | 1649, | C₂₂H₃₄N₂O₇Na, | 5.6, 3.4 Hz, 1H), 3.94 (s, 3H), 3.64 (dd, J = 8.5, |
| | | 1525, | 461.2258; | 6.4 Hz, 1H), 3.40 (s, 3H), 3.31 (dd, J = 8.5, |
| | | 1098 | found, | 6.5 Hz, 1H), 3.18 (dd, J = 9.3, 8.3 Hz, |
| | | | 461.2277 | 1H), 3.04 (ddd, J = 8.5, 5.9, 3.0 Hz, 1H), |
| | | | | 2.23-1.99 (m, 2H), 1.90-1.73 (m, 2H), |
| | | | | 1.71-1.45 (m, 3H), 1.43-1.33 (m, 1H), 1.39 (d, J = 6.3 Hz, |
| | | | | 3H), 1.26 (td, J = 8.6, 8.0, 4.2 Hz, |
| | | | | 1H), 0.92 (d, J = 6.7 Hz, 6H) |
| 93 | — | — | HRMS-ESI | ¹H NMR (400 MHz, CDCl₃) δ 12.12 (s, 1H), |
| | | | (m/z) | 8.70 (d, J = 7.5 Hz, 1H), 8.01 (d, J = 5.2 Hz, |
| | | | [M + H]⁺ | 1H), 7.25 (tt, J = 7.5, 2.2 Hz, 2H), 7.02 (dd, J = 8.8, |
| | | | calcd for | 0.9 Hz, 2H), 6.97-6.88 (m, 1H), |
| | | | C₂₇H₃₇N₂O₇, | 6.87 (d, J = 5.2 Hz, 1H), 5.16 (dq, J = 9.3, 6.3 Hz, |
| | | | 501.2595; | 1H), 4.78 (ddd, J = 7.6, 5.5, 3.4 Hz, 1H), |
| | | | found, | 4.30 (t, J = 8.8 Hz, 1H), 3.94 (s, 3H), 3.29 (tt, J = 6.0, |
| | | | 501.2613 | 2.9 Hz, 1H), 3.22 (dd, J = 8.9, 6.4 Hz, |
| | | | | 1H), 3.17 (dd, J = 8.9, 6.5 Hz, 1H), 2.23 (ddt, |
| | | | | J = 14.9, 7.5, 3.6 Hz, 1H), 2.10 (ddt, J = 14.8, |
| | | | | 8.0, 4.8 Hz, 1H), 1.90 (ddd, J = 14.5, 8.6, 4.3 Hz, |
| | | | | 1H), 1.68 (ddp, J = 12.4, 8.9, 4.2 Hz, 2H), |
| | | | | 1.62-1.48 (m, 3H), 1.35 (d, J = 6.3 Hz, 3H), |
| | | | | 1.33-1.24 (m, 1H), 0.73 (d, J = 6.7 Hz, 3H), |
| | | | | 0.66 (d, J = 6.7 Hz, 3H) |
| | | | | ¹³C NMR (101 MHz, CDCl₃) δ 171.84, |
| | | | | 168.70, 159.52, 155.35, 148.71, 140.58, |
| | | | | 130.47, 129.20, 121.03, 116.04, 109.48, |
| | | | | 82.46, 82.06, 77.78, 72.66, 56.07, 51.57, |
| | | | | 28.74, 28.25, 27.22, 22.31, 21.82, 19.29, |
| | | | | 19.18, 18.34 |
| 94 | — | — | HRMS-ESI | ¹H NMR (400 MHz, CDCl₃) δ |
| | | | (m/z) | 12.15-12.06 (m, 1H), 8.73 (d, J = 7.6 Hz, 1H), |
| | | | [M + H]⁺ | 8.12-8.04 (m, 2H), 8.04-7.98 (m, 1H), 7.57 (tt, J = 6.9, |
| | | | calcd for | 1.3 Hz, 1H), 7.51-7.41 (m, 2H), 6.88 (d, J = 5.2 Hz, |
| | | | C₂₉H₃₉N₂O₈, | 1H), 5.16-4.99 (m, 2H), |
| | | | 543.2701; | 4.86-4.72 (m, 1H), 3.95 (s, 3H), 3.71-3.54 (m, 2H), |
| | | | found, | 3.46 (t, J = 9.0 Hz, 1H), 2.22 (dtt, J = 14.6, |
| | | | 543.2700 | 9.9, 5.9 Hz, 1H), 2.15-1.96 (m, 2H), |
| | | | | 1.82-1.70 (m, 1H), 1.68-1.54 (m, 3H), 1.45 (d, J = 6.3 Hz, |
| | | | | 3H), 1.33 (dddd, J = 22.3, 17.8, 9.2, |
| | | | | 2.4 Hz, 4H), 1.17-1.01 (m, 3H), |
| | | | | 0.85-0.59 (m, 3H) |
| | | | | ¹³C NMR (101 MHz, CDCl₃) δ 171.59, |
| | | | | 168.75, 165.66, 155.32, 148.66, 140.63, |
| | | | | 132.91, 130.44, 129.53, 128.37, 109.48, |
| | | | | 82.94, 76.45, 74.05, 72.93, 56.07, 51.47, |
| | | | | 29.77, 28.95, 28.12, 22.37, 22.00, 21.51, |
| | | | | 18.05, 13.83 |
| 95 | 197-198 | — | ESIMS | ¹H NMR (400 MHz, CDCl₃) δ 12.08 (s, 1H), |
| | | | m/z 501 | 8.88 (d, J = 6.8 Hz, 1H), 7.99 (d, J = 5.1 Hz, |
| | | | ([M + H]⁺) | 1H), 7.32-7.25 (m, 2H), 7.20 (t, J = 6.5 Hz, |
| | | | | 3H), 6.85 (d, J = 5.1 Hz, 1H), 5.16-5.06 (m, |
| | | | | 1H), 4.70-4.62 (m, 1H), 3.93 (s, 3H), 3.73 (t, |
| | | | | J = 10.9 Hz, 1H), 3.45 (dd, J = 8.2, 6.5 Hz, |
| | | | | 1H), 3.39-3.24 (m, 4H), 3.23-3.17 (m, |
| | | | | 1H), 3.08 (t, J = 9.3 Hz, 1H), 2.56 (td, J = 11.6, |
| | | | | 3.8 Hz, 1H), 2.17-2.02 (m, 2H), |
| | | | | 1.89 (dt, J = 13.1, 6.5 Hz, 2H), 1.43 (d, J = 6.4 Hz, |
| | | | | 3H), 0.96 (dd, J = 6.7, 1.1 Hz, 6H) |
| | | | | ¹³C NMR (101 MHz, CDCl₃) δ 171.42, |
| | | | | 168.80, 155.26, 148.61, 140.64, 139.98, |
| | | | | 130.50, 129.14, 128.44, 126.08, 109.43, |
| | | | | 83.82, 78.79, 74.85, 68.77, 65.03, 56.06, |
| | | | | 49.91, 46.59, 35.10, 29.14, 28.99, 19.55, |
| | | | | 19.52, 18.54 |
| 96 | — | — | HRMS-ESI | ¹H NMR (400 MHz, CDCl₃) δ 12.11 (s, 1H), |
| | | | (m/z) | 8.71 (d, J = 7.4 Hz, 1H), 8.01 (d, J = 5.2 Hz, |
| | | | [M + H]⁺ | 1H), 7.36-7.20 (m, 2H), 6.99-6.80 (m, |
| | | | calcd for | 4H), 5.04 (dq, J = 9.4, 6.3 Hz, 1H), 4.78 (ddd, |
| | | | C₂₈H₃₉N₂O₇, | J = 7.6, 5.3, 3.5 Hz, 1H), 4.24-4.08 (m, 1H), |
| | | | 515.2752; | 3.94 (s, 3H), 3.82 (dt, J = 8.7, 6.6 Hz, 1H), |
| | | | found, | 3.57 (dt, J = 8.7, 6.7 Hz, 1H), 3.49-3.40 (m, |
| | | | 515.2770 | 1H), 2.34-2.17 (m, 1H), 2.07 (dq, J = 15.0, |
| | | | | 5.4 Hz, 1H), 2.01-1.88 (m, 1H), |
| | | | | 1.77-1.64 (m, 1H), 1.62-1.51 (m, 3H), 1.51-1.35 (m, |
| | | | | 5H), 1.21 (ttd, J = 13.0, 6.7, 2.4 Hz, 5H), |
| | | | | 0.80 (t, J = 7.0 Hz, 3H) |
| | | | | ¹³C NMR (101 MHz, CDCl₃) δ 171.90, |
| | | | | 168.73, 158.09, 155.35, 148.70, 140.60, |
| | | | | 130.48, 129.40, 120.69, 115.71, 115.55, |
| | | | | 109.47, 83.81, 80.83, 74.07, 72.63, 56.08, |
| | | | | 51.32, 29.86, 28.22, 28.12, 22.46, 21.80, |
| | | | | 21.63, 18.17, 13.96 |
| 97 | — | — | HRMS-ESI | ¹H NMR (400 MHz, CDCl₃) δ 12.11 (s, 1H), |
| | | | (m/z) | 8.70 (d, J = 7.4 Hz, 1H), 8.01 (dd, J = 5.2, 1.3 Hz, |
| | | | [M + H]⁺ | 1H), 7.36-7.13 (m, 2H), 7.00-6.82 (m, |
| | | | calcd for | 4H), 5.04 (dq, J = 9.4, 6.3 Hz, 1H), 4.78 (ddd, |
| | | | C₂₆H₃₅N₂O₇, | J = 1.1, 5.3, 3.5 Hz, 1H), 4.21-4.08 (m, 1H), |
| | | | 487.2439; | 3.94 (s, 3H), 3.80 (dt, J = 8.7, 6.7 Hz, 1H), |
| | | | found, | 3.54 (dt, J = 8.7, 6.7 Hz, 1H), 3.50-3.41 (m, |
| | | | 487.2454 | 1H), 2.31-2.17 (m, 1H), 2.07 (dq, J = 15.1, |
| | | | | 5.4 Hz, 1H), 2.01-1.85 (m, 1H), |
| | | | | 1.80-1.64 (m, 1H), 1.57 (dt, J = 11.8, 6.0 Hz, 3H), |
| | | | | 1.50-1.38 (m, 4H), 1.38-1.16 (m, 2H), 0.79 (t, J = 7.4 Hz, |
| | | | | 3H) |
| | | | | ¹³C NMR (101 MHz, CDCl₃) δ 171.90, |
| | | | | 168.73, 158.09, 155.36, 148.71, 140.60, |
| | | | | 129.42, 120.70, 115.73, 109.47, 83.78, 80.91, |
| | | | | 75.66, 72.64, 56.08, 51.32, 28.10, 26.87, |
| | | | | 23.35, 21.79, 21.64, 18.17, 10.56 |
| 98 | — | (Neat) | HRMS-ESI | ¹H NMR (400 MHz, CDCl₃) δ 12.08 (s, 1H), |
| | | 3369, | (m/z) | 8.72 (d, J = 7.4 Hz, 1H), 8.02 (d, J = 5.2 Hz, |
| | | 2941, | [M + H]⁺ | 1H), 7.29-7.13 (m, 4H), 7.01-6.85 (m, |
| | | 1735, | calcd for | 5H), 6.76-6.67 (m, 2H), 5.27 (dq, J = 9.3, |
| | | 1649, | C₂₉H₃₃N₂O₇, | 6.3 Hz, 1H), 4.83 (ddd, J = 7.6, 5.3, 3.4 Hz, |
| | | 1490, | 521.2282; | 1H), 4.52 (t, J = 8.8 Hz, 1H), 4.30 (ddd, J = 8.7, |
| | | 1228, | found, | 7.0, 2.5 Hz, 1H), 3.94 (s, 3H), |
| | | 1194 | 521.2304 | 2.37-2.25 (m, 1H), 2.19-2.03 (m, 2H), 1.86-1.55 (m, |
| | | | | 4H), 1.43 (d, J = 6.3 Hz, 3H), 1.39-1.27 (m, |
| | | | | 1H) |
| 99 | — | (Neat) | HRMS-ESI | ¹H NMR (400 MHz, CDCl₃) δ 12.12 (d, J = 0.6 Hz, |
| | | 3367, | (m/z) | 1H), 8.67 (d, J = 7.5 Hz, 1H), |
| | | 2940, | [M + H]⁺ | 8.00 (dd, J = 5.2, 0.8 Hz, 1H), 6.87 (d, J = 5.2 Hz, |
| | | 2828, | calcd for | 1H), 4.92 (dq, J = 9.0, 6.3 Hz, 1H), 4.74 (ddd, |
| | | 1732, | C₁₉H₂₉N₂O₇, | J = 7.5, 5.5, 3.4 Hz, 1H), 3.95 (s, 3H), 3.58 (s, |
| | | 1649, | 397.1969; | 3H), 3.43 (s, 3H), 3.11 (t, J = 8.6 Hz, 1H), |
| | | 1524, | found, | 3.04 (ddd, J = 8.4, 5.9, 2.9 Hz, 1H), 2.19 (ddt, |
| | | 1101 | 397.1974 | J = 15.3, 7.8, 3.9 Hz, 1H), 2.13-2.01 (m, |
| | | | | 1H), 1.79 (ddt, J = 14.3, 8.6, 5.8 Hz, 1H), |
| | | | | 1.71-1.48 (m, 3H), 1.45-1.35 (m, 1H), 1.40 (d, J = 6.3 Hz, |
| | | | | 3H), 1.31-1.20 (m, 1H) |
| 100 | — | (Neat) | HRMS-ESI | ¹H NMR (400 MHz, CDCl₃) δ 12.11 (d, J = 0.6 Hz, |
| | | 3370, | (m/z) | 1H), 8.70 (d, J = 7.5 Hz, 1H), 8.02 (d, |
| | | 2937, | [M + Na]⁺ | J = 5.2 Hz, 1H), 7.30-7.23 (m, 2H), |
| | | 2874, | calcd for | 7.06-7.00 (m, 2H), 6.97-6.91 (m, 1H), 6.88 (dd, J = 5.3, |
| | | 1734, | C₂₆H₃₄N₂O₇Na, | 0.7 Hz, 1H), 5.16 (dq, J = 9.3, 6.3 Hz, |
| | | 1649, | 509.2258; | 1H), 4.78 (ddd, J = 7.5, 5.4, 3.5 Hz, 1H), |
| | | 1524, | found, | 4.28 (t, J = 8.9 Hz, 1H), 3.95 (s, 3H), |
| | | 1195 | 509.2281 | 3.45-3.33 (m, 2H), 3.30 (ddd, J = 8.8, 6.0, 3.2 Hz, 1H), |
| | | | | 2.28-2.17 (m, 1H), 2.17-2.05 (m, 1H), |
| | | | | 1.97-1.85 (m, 1H), 1.75-1.61 (m, 2H), |
| | | | | 1.61-1.49 (m, 2H), 1.40-1.25 (m, 6H), 0.70 (t, J = 7.4 Hz, |
| | | | | 3H) |
| 101 | — | (Neat) | HRMS-ESI | ¹H NMR (400 MHz, CDCl₃) δ 12.09 (s, 1H), |
| | | 2956, | (m/z) | 8.83 (d, J = 8.2 Hz, 1H), 8.01 (d, J = 5.2 Hz, |
| | | 2872, | [M]⁺ | 1H), 7.31-7.09 (m, 5H), 6.87 (d, J = 5.2 Hz, |
| | | 1743, | calcd for | 1H), 5.20 (dq, J = 9.0, 6.3 Hz, 1H), 4.88 (ddd, |
| | | 1650, | C₂₇H₃₆N₂O₇, | J = 8.2, 3.7, 1.3 Hz, 1H), 3.93 (s, 3H), |
| | | 1527, | 500.2523; | 3.89-3.72 (m, 2H), 3.47 (dd, J = 8.4, 6.2 Hz, 1H), |
| | | 1262, | found, | 3.32 (dd, J = 13.1, 4.1 Hz, 1H), 3.27 (dd, J = 8.4, |
| | | 1084 | 500.2538 | 6.6 Hz, 1H), 3.03 (dt, J = 12.1, 3.7 Hz, |
| | | | | 1H), 2.98 (dd, J = 9.1, 7.6 Hz, 1H), |
| | | | | 2.68-2.57 (m, 1H), 2.22 (dd, J = 13.2, 11.4 Hz, |
| | | | | 1H), 2.12-1.99 (m, 1H), 1.93-1.78 (m, |
| | | | | 2H), 1.40 (d, J = 6.3 Hz, 3H), 1.33 (dddt, J = 13.4, |
| | | | | 6.7, 3.8, 2.5 Hz, 1H), 0.94 (dd, J = 6.7, |
| | | | | 3.8 Hz, 6H) |
| | | | | ¹³C NMR (101 MHz, CDCl₃) δ 168.89, |
| | | | | 168.76, 155.43, 148.77, 140.77, 140.47, |
| | | | | 130.25, 129.24, 128.40, 125.91, 109.44, |
| | | | | 85.69, 80.70, 74.86, 68.92, 67.93, 56.10, |
| | | | | 53.73, 39.38, 38.63, 32.47, 29.14, 19.57, |
| | | | | 19.52, 19.07 |
| 102 | — | — | HRMS-ESI | ¹H NMR (400 MHz, CDCl₃) δ 12.13 (s, 1H), |
| | | | (m/z) | 8.65 (d, J = 1.5 Hz, 1H), 7.97 (d, J = 5.2 Hz, |
| | | | [M + H]⁺ | 1H), 7.35-7.22 (m, 2H), 7.18 (d, J = 7.2 Hz, |
| | | | calcd for | 3H), 6.84 (d, J = 5.2 Hz, 1H), 5.03 (dq, J = 9.2, |
| | | | C₂₈H₃₉N₂O₆, | 6.3 Hz, 1H), 4.72 (ddd, J = 7.7, 5.5, 3.4 Hz, |
| | | | 499.2803; | 1H), 3.92 (s, 3H), 3.48-3.34 (m, 2H), |
| | | | found, | 3.27 (dd, J = 13.2, 3.3 Hz, 1H), 3.07 (t, J = 9.3 Hz, |
| | | | 499.2827 | 1H), 2.28-2.10 (m, 2H), 1.99 (ddd, J = 11.2, |
| | | | | 9.6, 5.4 Hz, 1H), 1.88 (dp, J = 13.2, |
| | | | | 6.6 Hz, 1H), 1.82-1.67 (m, 1H), 1.43 (d, J = 6.3 Hz, |
| | | | | 4H), 1.38 (dt, J = 12.5, 5.8 Hz, 2H), |
| | | | | 1.34-1.14 (m, 3H), 0.95 (dd, J = 6.7, 1.4 Hz, |
| | | | | 6H) |
| | | | | ¹³C NMR (101 MHz, CDCl₃) δ 171.94, |
| | | | | 168.70, 155.34, 148.72, 141.00, 140.53, |
| | | | | 130.58, 129.11, 128.27, 125.78, 109.45, |
| | | | | 84.64, 79.71, 75.74, 56.04, 51.46, 45.56, |
| | | | | 38.33, 29.17, 27.10, 26.18, 24.42, 21.96, |
| | | | | 19.55, 19.52, 18.47 |
| 103 | — | — | HRMS-ESI | ¹H NMR (400 MHz, CDCl₃) δ 12.14 (s, 1H), |
| | | | (m/z) | 8.70 (d, J = 7.4 Hz, 1H), 8.00 (dd, J = 5.2, 2.6 Hz, |
| | | | [M + H]⁺ | 1H), 6.89 (d, J = 5.2 Hz, 1H), 5.05 (dq, J = 12.3, |
| | | | calcd for | 6.2 Hz, 1H), 4.87 (t, J = 9.5 Hz, 1H), |
| | | | C₂₄H₃₇N₂O₇, | 4.80 (dq, J = 7.9, 4.7, 4.1 Hz, 1H), 3.94 (s, |
| | | | 465.2595; | 3H), 2.60 (hept, J = 7.0 Hz, 1H), |
| | | | found, | 2.36-2.21 (m, 1H), 2.16-2.01 (m, 1H), 1.58 (tt, J = 25.8, |
| | | | 465.2622 | 11.5 Hz, 5H), 1.30 (s, 3H), 1.24 (d, J = 6.3 Hz, |
| | | | | 4H), 1.21 (d, J = 7.0 Hz, 6H), |
| | | | | 1.19-1.14 (m, 1H), 0.92-0.80 (m, 4H) |
| | | | | ¹³C NMR (101 MHz, CDCl₃) δ 176.07, |
| | | | | 171.86, 168.68, 155.29, 148.65, 140.50, |
| | | | | 130.43, 109.44, 74.13, 56.01, 51.35, 41.50, |
| | | | | 34.32, 34.22, 31.52, 26.87, 26.74, 24.69, |
| | | | | 22.59, 21.71, 19.55, 18.95, 17.93, 14.28, |
| | | | | 14.07 |
| 104 | — | (Neat) | HRMS-ESI | ¹H NMR (400 MHz, CDCl₃) δ 12.06 (s, 1H), |
| | | 3367, | (m/z) | 8.54 (d, J = 7.8 Hz, 1H), 7.99 (d, J = 5.1 Hz, |
| | | 2937, | [M + H]⁺ | 1H), 6.87 (d, J = 5.2 Hz, 1H), 5.00 (dq, J = 9.2, |
| | | 1740, | calcd for | 6.2 Hz, 1H), 4.90-4.80 (m, 1H), 3.94 (s, |
| | | 1649, | C₂₀H₂₉N₂O₇, | 3H), 3.91-3.81 (m, 1H), 3.60 (dd, J = 9.3, |
| | | 1525, | 409.1969; | 7.9 Hz, 1H), 2.23-2.09 (m, 1H), |
| | | 1262, | found, | 2.03-1.84 (m, 2H), 1.82-1.60 (m, 3H), 1.52-1.39 (m, |
| | | 1195 | 409.2005 | 1H), 1.43 (d, J = 6.4 Hz, 3H), 1.42 (s, 3H), |
| | | | | 1.37 (s, 3H), 1.36-1.23 (m, 1H) |
| 105 | — | — | HRMS-ESI | ¹H NMR (400 MHz, CDCl₃) δ 12.17 (s, 1H), |
| | | | (m/z) | 8.71 (d, J = 7.4 Hz, 1H), 7.99 (dd, J = 5.2, 1.5 Hz, |
| | | | [M + H]⁺ | 1H), 6.87 (d, J = 5.1 Hz, 1H), 4.95 (dq, J = 12.8, |
| | | | calcd for | 6.3 Hz, 1H), 4.74 (dt, J = 7.6, 4.1 Hz, |
| | | | C₂₄H₃₉N₂O₆, | 1H), 3.93 (s, 3H), 3.32 (p, J = 8.1 Hz, 2H), |
| | | | 451.2803; | 2.95 (t, J = 9.2 Hz, 1H), 2.30-2.15 (m, 1H), |
| | | | found, | 2.15-1.96 (m, 1H), 1.84 (dp, J = 13.1, 6.5 Hz, |
| | | | 451.2809 | 1H), 1.64 (tt, J = 10.0, 4.4 Hz, 1H), |
| | | | | 1.59-1.48 (m, 4H), 1.48-1.31 (m, 5H), |
| | | | | 1.31-1.07 (m, 4H), 0.99-0.84 (m, 9H) |
| | | | | ¹³C NMR (101 MHz, CDCl₃) δ 171.87, |
| | | | | 168.69, 155.28, 148.63, 140.53, 130.51, |
| | | | | 128.98, 128.17, 125.25, 109.41, 84.55, 79.49, |
| | | | | 75.95, 56.02, 51.48, 43.09, 34.33, 29.10, |
| | | | | 27.32, 26.84, 24.90, 21.86, 20.17, 19.52, |
| | | | | 19.49, 18.48, 14.43 |
| 106 | — | — | HRMS-ESI | ¹H NMR (400 MHz, CDCl₃) δ 12.06 (s, 1H), |
| | | | (m/z) | 8.87 (d, J = 6.9 Hz, 1H), 7.99 (d, J = 5.2 Hz, |
| | | | [M + H]⁺ | 1H), 7.27-7.19 (m, 4H), 6.89-6.83 (m, |
| | | | calcd for | 5H), 5.06 (dq, J = 9.2, 6.3 Hz, 1H), 4.86 (d, J = 10.3 Hz, |
| | | | C₃₂H₃₉N₂O₁₀, | 1H), 4.72-4.64 (m, 1H), 4.60 (s, |
| | | | 611.2599; | 2H), 4.55 (d, J = 10.3 Hz, 1H), 3.94 (s, 3H), |
| | | | found, | 3.93-3.83 (m, 1H), 3.81 (s, 3H), 3.80 (s, |
| | | | 611.2601 | 3H), 3.66-3.52 (m, 2H), 3.51-3.38 (m, |
| | | | | 3H), 2.66-2.53 (m, 1H), 2.04-1.92 (m, |
| | | | | 1H), 1.38 (d, J = 6.3 Hz, 3H) |
| | | | | ¹³C NMR (101 MHz, CDCl₃) δ 171.26, |
| | | | | 168.85, 159.29, 159.27, 155.30, 148.64, |
| | | | | 140.67, 130.47, 130.33, 130.24, 129.69, |
| | | | | 129.50, 113.84, 113.81, 109.48, 83.53, 82.29, |
| | | | | 75.58, 72.85, 71.88, 69.22, 65.34, 56.08, |
| | | | | 55.30, 55.29, 49.95, 29.02, 18.52 |
| 107 | — | (Neat) | HRMS-ESI | ¹H NMR (400 MHz, CDCl₃) δ 12.13 (s, 1H), |
| | | 3370, | (m/z) | 8.67 (d, J = 7.5 Hz, 1H), 8.00 (d, J = 5.1 Hz, |
| | | 2942, | [M + H]⁺ | 1H), 6.87 (d, J = 5.2 Hz, 1H), 4.90 (dq, J = 9.3, |
| | | 1733, | calcd for | 6.4 Hz, 1H), 4.73 (ddd, J = 7.4, 5.3, 3.5 Hz, |
| | | 1649, | C₂₃H₃₅N₂O₇, | 1H), 4.30 (p, J = 4.6 Hz, 1H), 3.94 (s, |
| | | 1524, | 451.2439; | 3H), 3.41 (s, 3H), 3.30 (t, J = 8.7 Hz, 1H), |
| | | 1051 | found, | 2.99 (ddd, J = 8.2, 5.1, 3.3 Hz, 1H), |
| | | | 451.2447 | 2.19-2.01 (m, 2H), 1.85-1.63 (m, 9H), |
| | | | | 1.63-1.43 (m, 3H), 1.42-1.28 (m, 2H), 1.38 (d, J = 6.3 Hz, |
| | | | | 3H) |
| 108 | — | (Neat) | HRMS-ESI | ¹H NMR (400 MHz, CDCl₃) δ 12.13 (s, 1H), |
| | | 3361, | (m/z) | 8.68 (d, J = 7.4 Hz, 1H), 8.00 (d, J = 5.3 Hz, |
| | | 2944, | [M + H]⁺ | 1H), 6.87 (d, J = 5.2 Hz, 1H), 4.90 (dq, J = 9.2, |
| | | 1723, | calcd for | 6.4 Hz, 1H), 4.74 (ddd, J = 8.0, 5.4, 3.4 Hz, |
| | | 1650, | C₂₃H₃₅N₂O₇, | 1H), 4.14-4.05 (m, 1H), 3.94 (s, 3H), |
| | | 1525, | 451.2439; | 3.57 (s, 3H), 3.17 (ddd, J = 8.9, 6.4, 3.0 Hz, |
| | | 1129 | found, | 1H), 3.05 (t, J = 8.7 Hz, 1H), 2.26-2.14 (m, |
| | | | 451.244 | 1H), 2.12-2.00 (m, 1H), 1.86-1.46 (m, |
| | | | | 12H), 1.45-1.31 (m, 1H), 1.39 (d, J = 6.3 Hz, |
| | | | | 3H), 1.29-1.12 (m, 1H) |
| 109 | — | (Neat) | HRMS-ESI | ¹H NMR (400 MHz, CDCl₃) δ 12.11 (d, J = 0.6 Hz, |
| | | 3369, | (m/z) | 1H), 8.69 (d, J = 7.5 Hz, 1H), 8.01 (d, |
| | | 2944, | [M + H]⁺ | J = 5.2 Hz, 1H), 7.32-7.22 (m, 2H), |
| | | 1734, | calcd for | 6.96-6.85 (m, 4H), 5.03 (dq, J = 9.3, 6.3 Hz, 1H), |
| | | 1649, | C₂₈H₃₇N₂O₇, | 4.78 (ddd, J = 7.4, 5.3, 3.5 Hz, 1H), |
| | | 1524, | 513.2595; | 4.35-4.28 (m, 1H), 4.13 (ddd, J = 8.5, 5.8, 3.2 Hz, |
| | | 1239 | found, | 1H), 3.94 (s, 3H), 3.56 (dd, J = 9.2, 8.2 Hz, |
| | | | 513.2605 | 1H), 2.24-2.14 (m, 1H), 2.13-2.02 (m, |
| | | | | 1H), 2.01-1.87 (m, 2H), 1.76-1.24 (m, |
| | | | | 12H), 1.44 (d, J = 6.3 Hz, 3H) |
| 110 | — | (Neat) | HRMS-ESI | ¹H NMR (400 MHz, CDCl₃) δ 12.04 (s, 1H), |
| | | 3345, | (m/z) | 8.91 (d, J = 6.9 Hz, 1H), 8.03 (d, J = 5.2 Hz, |
| | | 2936, | [M + H]⁺ | 1H), 7.29-7.17 (m, 4H), 7.02-6.87 (m, |
| | | 1733, | calcd for | 5H), 6.77-6.72 (m, 2H), 5.38 (dq, J = 9.6, |
| | | 1649, | C₂₈H₃₁N₂O₈, | 6.3 Hz, 1H), 4.76 (ddd, J = 7.0, 5.1, 3.9 Hz, |
| | | 1483, | 523.2075; | 1H), 4.52 (dd, J = 9.6, 8.6 Hz, 1H), |
| | | 1266, | found, | 4.43-4.32 (m, 1H), 4.01-3.92 (m, 1H), 3.95 (s, |
| | | 1210 | 523.2081 | 3H), 3.81 (dd, J = 9.8, 8.0 Hz, 1H), 3.64 (dd, J = 9.8, |
| | | | | 1.4 Hz, 1H), 3.44 (ddd, J = 12.4, 4.2, |
| | | | | 3.0 Hz, 1H), 2.73-2.59 (m, 1H), |
| | | | | 2.11-1.99 (m, 1H), 1.42 (d, J = 6.3 Hz, 3H) |
| 111 | — | (Neat) | HRMS-ESI | ¹H NMR (400 MHz, CDCl₃) δ 12.09 (s, 1H), |
| | | 3368, | (m/z) | 8.69 (d, J = 7.5 Hz, 1H), 8.01 (d, J = 5.4 Hz, |
| | | 2958, | [M + H]⁺ | 1H), 6.87 (d, J = 5.2 Hz, 1H), 5.00 (dq, J = 9.3, |
| | | 1739, | calcd for | 6.3 Hz, 1H), 4.77 (ddd, J = 7.4, 5.1, 3.5 Hz, |
| | | 1649, | C₂₅H₃₉N₂O₉, | 1H), 4.62 (ddd, J = 9.1, 6.4, 3.2 Hz, 1H), |
| | | 1525, | 511.265; | 4.09 (t, J = 6.7 Hz, 2H), 3.95 (s, 3H), |
| | | 1255 | found, | 3.50 (dd, J = 8.3, 6.4 Hz, 1H), 3.34 (dd, J = 8.3, 6.3 Hz, |
| | | | 511.2646 | 1H), 3.31-3.25 (m, 1H), 2.23-2.02 (m, |
| | | | | 2H), 2.01-1.87 (m, 2H), 1.85-1.46 (m, |
| | | | | 7H), 1.41 (d, J = 6.2 Hz, 3H), 0.97 (t, J = 7.4 Hz, |
| | | | | 3H), 0.89 (d, J = 6.7 Hz, 3H), 0.88 (d, J = 6.7 Hz, |
| | | | | 3H) |
| 112 | — | (Neat) | HRMS-ESI | ¹H NMR (400 MHz, CDCl₃) δ 12.09 (s, 1H), |
| | | 3376, | (m/z) | 8.88 (d, J = 8.2 Hz, 1H), 8.00 (d, J = 5.2 Hz, |
| | | 2932, | [M + H]⁺ | 1H), 6.87 (d, J = 5.2 Hz, 1H), 5.09 (dq, J = 9.4, |
| | | 2869, | calcd for | 6.2 Hz, 1H), 4.91 (ddd, J = 8.4, 3.7, 1.7 Hz, |
| | | 1737, | C₂₁H₃₃N₂O₆, | 1H), 4.03-3.88 (m, 5H), 3.59 (ddd, J = 11.5, |
| | | 1649, | 409.2333; | 9.8, 3.3 Hz, 1H), 3.43 (ddd, J = 11.5, |
| | | 1526, | found, | 4.7, 3.4 Hz, 1H), 1.77-1.06 (m, 13H), |
| | | 1241, | 409.2334 | 0.89 (appt, J = 6.4 Hz, 6H) |
| | | 1117 | | |
| 113 | — | (Neat) | HRMS-ESI | ¹H NMR (400 MHz, CDCl₃) δ 12.06 (s, 1H), |
| | | 3371, | (m/z) | 8.88 (d, J = 7.0 Hz, 1H), 8.01 (d, J = 5.2 Hz, |
| | | 2930, | [M + H]⁺ | 1H), 7.31-7.25 (m, 2H), 7.04-6.92 (m, |
| | | 1721, | calcd for | 3H), 6.88 (d, J = 5.2 Hz, 1H), 5.22 (dq, J = 9.7, |
| | | 1641, | C₂₃H₂₉N₂O₈, | 6.3 Hz, 1H), 4.71 (ddd, J = 7.0, 5.2, 4.0 Hz, |
| | | 1242, | 461.1918; | 1H), 4.28 (dd, J = 9.7, 8.4 Hz, 1H), |
| | | 1211 | found, | 3.98-3.87 (m, 1H), 3.94 (s, 3H), 3.63 (dd, J = 9.8, |
| | | | 461.1925 | 8.1 Hz, 1H), 3.59-3.52 (m, 1H), 3.48 (ddd, J = 12.2, |
| | | | | 4.6, 3.0 Hz, 1H), 3.36 (s, 3H), |
| | | | | 3.34-3.28 (m, 1H), 2.67-2.55 (m, 1H), |
| | | | | 2.07-1.97 (m, 1H), 1.35 (d, J = 6.3 Hz, 3H) |
| 114 | — | (Neat) | HRMS-ESI | ¹H NMR (400 MHz, CDCl₃) δ 12.06 (s, 1H), |
| | | 3345, | (m/z) | 8.89 (d, J = 6.9 Hz, 1H), 8.01 (d, J = 5.2 Hz, |
| | | 2925, | [M + H]⁺ | 1H), 7.31-7.22 (m, 2H), 7.06-6.98 (m, |
| | | 1720, | calcd for | 2H), 6.98-6.91 (m, 1H), 6.88 (d, J = 5.2 Hz, |
| | | 1645, | C₂₅H₃₃N₂O₈, | 1H), 5.22 (dq, J = 9.6, 6.3 Hz, 1H), |
| | | 1206 | 489.2231; | 4.75-4.68 (m, 1H), 4.30 (dd, J = 9.7, 8.4 Hz, 1H), |
| | | | found, | 3.98-3.88 (m, 1H), 3.94 (s, 3H), 3.65 (dd, J = 9.7, |
| | | | 489.2239 | 8.4 Hz, 1H), 3.55 (dd, J = 9.6, 1.8 Hz, |
| | | | | 1H), 3.53-3.36 (m, 4H), 2.70-2.54 (m, |
| | | | | 1H), 2.07-1.95 (m, 1H), 1.42-1.28 (m, |
| | | | | 2H), 1.34 (d, J = 6.4 Hz, 3H), 0.70 (t, J = 7.4 Hz, |
| | | | | 3H) |
| 115 | — | — | ESIMS | ¹H NMR (400 MHz, CDCl₃) δ 12.06 (d, J = 0.6 Hz, |
| | | | m/z 489.4 | 1H), 8.90 (d, J = 6.9 Hz, 1H), 8.01 (d, |
| | | | ([M + H]⁺) | J = 5.2 Hz, 1H), 7.32-7.24 (m, 2H), |
| | | | | 6.99-6.92 (m, 3H), 6.87 (d, J = 5.2 Hz, 1H), |
| | | | | 5.15 (dq, J = 9.8, 6.3 Hz, 1H), 4.71 (ddd, J = 6.9, |
| | | | | 5.0, 3.9 Hz, 1H), 4.26-4.18 (m, 1H), 3.94 (s, |
| | | | | 3H), 3.94-3.88 (m, 1H), 3.88-3.81 (m, |
| | | | | 1H), 3.70 (dd, J = 9.7, 8.1 Hz, 1H), |
| | | | | 3.59-3.48 (m, 2H), 3.44 (dd, J = 9.8, 8.6 Hz, 1H), |
| | | | | 3.40-3.33 (m, 1H), 2.69-2.54 (m, 1H), |
| | | | | 2.05-1.95 (m, 1H), 1.55-1.45 (m, 2H), 1.44 (d, J = 6.3 Hz, |
| | | | | 3H), 0.83 (t, J = 7.4 Hz, 3H) |
| | | | | ¹³C NMR (101 MHz, CDCl₃) δ 171.17, |
| | | | | 168.90, 157.57, 155.32, 148.66, 140.69, |
| | | | | 130.47, 129.57, 121.13, 115.53, 109.50, |
| | | | | 83.56, 80.16, 75.71, 71.66, 68.46, 65.27, |
| | | | | 56.08, 49.90, 28.79, 23.40, 18.43, 10.62 |
| 116 | — | (Neat) | HRMS-ESI | ¹H NMR (400 MHz, CDCl₃) δ 12.08 (s, 1H), |
| | | 3363, | (m/z) | 8.87 (d, J = 7.0 Hz, 1H), 8.00 (d, J = 5.2 Hz, |
| | | 2955, | [M + H]⁺ | 1H), 6.87 (d, J = 5.2 Hz, 1H), 5.05-4.95 (m, |
| | | 1728, | calcd for | 1H), 4.71-4.61 (m, 1H), 3.94 (s, 3H), |
| | | 1649, | C₂₄H₃₉N₂O₈, | 3.92-3.82 (m, 1H), 3.70 (dd, J = 8.5, 6.0 Hz, 1H), |
| | | 1525, | 483.2701; | 3.57-3.50 (m, 1H), 3.48-3.41 (m, 1H), |
| | | 1099 | found, | 3.41-3.08 (m, 6H), 2.66-2.53 (m, 1H), |
| | | | 483.2705 | 2.04-1.90 (m, 1H), 1.89-1.76 (m, 2H), 1.38 (d, J = 6.3 Hz, |
| | | | | 3H), 0.97-0.85 (m, 12H) |
| 117 | — | (Neat) | HRMS-ESI | ¹H NMR (400 MHz, CDCl₃) δ 12.03 (s, 1H), |
| | | 3357, | (m/z) | 8.86 (d, J = 7.1 Hz, 1H), 8.02 (d, J = 5.2 Hz, |
| | | 2935, | [M + H]⁺ | 1H), 7.30-7.23 (m, 2H), 6.98-6.92 (m, |
| | | 1739, | calcd for | 3H), 6.88 (d, J = 5.2 Hz, 1H), 5.26 (dq, J = 9.6, |
| | | 1722, | C₂₆H₃₃N₂O₉, | 6.5 Hz, 1H), 5.11-5.05 (m, 1H), |
| | | 1643, | 517.2181; | 4.73 (ddd, J = 7.1, 5.7, 4.0 Hz, 1H), |
| | | 1203 | found, | 4.58-4.50 (m, 1H), 3.95 (s, 3H), 3.95-3.87 (m, 1H), |
| | | | 517.2189 | 3.75 (dd, J = 9.8, 8.0 Hz, 1H), 3.53-3.45 (m, |
| | | | | 2H), 2.63-2.52 (m, 1H), 2.25-2.14 (m, |
| | | | | 1H), 2.11-2.00 (m, 1H), 1.37 (d, J = 6.4 Hz, |
| | | | | 3H), 0.93 (d, J = 7.0 Hz, 3H), 0.86 (d, J = 7.0 Hz, |
| | | | | 3H) |
| 118 | — | (Neat) | HRMS-ESI | ¹H NMR (400 MHz, CDCl₃) δ 12.06 (s, 1H), |
| | | 3352, | (m/z) | 8.89 (d, J = 6.9 Hz, 1H), 8.01 (d, J = 5.2 Hz, |
| | | 2953, | [M + H]⁺ | 1H), 7.29-7.22 (m, 2H), 7.03-6.98 (m, |
| | | 2845, | calcd for | 2H), 6.96-6.91 (m, 1H), 6.88 (d, J = 5.2 Hz, |
| | | 1736, | C₂₆H₃₅N₂O₈, | 1H), 5.22 (dq, J = 9.6, 6.3 Hz, 1H), 4.71 (ddd, |
| | | 1528, | 503.2388; | J = 7.0, 5.1, 4.0 Hz, 1H), 4.30 (dd, J = 9.7, 8.4 Hz, |
| | | 1209, | found, | 1H), 3.96-3.88 (m, 1H), 3.94 (s, 3H), |
| | | 1107 | 503.2398 | 3.65 (dd, J = 9.7, 8.3 Hz, 1H), 3.55 (dd, J = 9.7, |
| | | | | 1.8 Hz, 1H), 3.46 (ddd, J = 12.3, 4.5, 3.0 Hz, |
| | | | | 1H), 3.42-3.34 (m, 1H), 3.29 (dd, J = 8.9, |
| | | | | 6.4 Hz, 1H), 3.23 (dd, J = 8.9, 6.4 Hz, |
| | | | | 1H), 2.69-2.57 (m, 1H), 2.06-1.97 (m, |
| | | | | 1H), 1.58 (hept, J = 6.7 Hz, 1H), 1.34 (d, J = 6.3 Hz, |
| | | | | 3H), 0.71 (d, J = 6.7 Hz, 3H), 0.66 (d, |
| | | | | J = 6.7 Hz, 3H) |
| 119 | — | — | ESIMS | ¹H NMR (400 MHz, CDCl₃) δ 12.06 (s, 1H), |
| | | | m/z 503.4 | 8.90 (d, J = 6.9 Hz, 1H), 8.02 (d, J = 5.2 Hz, |
| | | | ([M + H]⁺) | 1H), 7.32-7.26 (m, 2H), 6.99-6.91 (m, |
| | | | | 3H), 6.88 (d, J = 5.2 Hz, 1H), 5.15 (dq, J = 9.8, |
| | | | | 6.3 Hz, 1H), 4.71 (ddd, J = 6.9, 5.0, 3.9 Hz, |
| | | | | 1H), 4.27-4.19 (m, 1H), 3.94 (s, 3H), |
| | | | | 3.94-3.87 (m, 1H), 3.73-3.66 (m, 2H), |
| | | | | 3.51 (dd, J = 9.6, 1.4 Hz, 1H), 3.46-3.41 (m, 1H), |
| | | | | 3.41-3.32 (m, 2H), 2.69-2.54 (m, 1H), |
| | | | | 2.05-1.95 (m, 1H), 1.73 (hept, J = 6.7 Hz, 1H), |
| | | | | 1.43 (d, J = 6.3 Hz, 3H), 0.84 (d, J = 6.7 Hz, |
| | | | | 3H), 0.80 (d, J = 6.7 Hz, 3H) |
| | | | | ¹³C NMR (101 MHz, CDCl₃) δ 171.18, |
| | | | | 168.89, 157.46, 155.32, 148.67, 140.69, |
| | | | | 130.47, 129.55, 121.08, 115.48, 109.50, |
| | | | | 83.41, 80.69, 80.06, 71.72, 68.42, 65.28, |
| | | | | 56.08, 49.91, 29.00, 28.81, 19.45, 19.38, |
| | | | | 18.47 |
| 120 | — | — | HRMS-ESI | ¹H NMR (400 MHz, CDCl₃) δ 11.88 (s, 1H), |
| | | | (m/z) | 8.70 (d, J = 7.5 Hz, 1H), 8.12 (dd, J = 4.3, 1.5 Hz, |
| | | | [M + H]⁺ | 1H), 7.36 (dd, J = 8.5, 4.3 Hz, 1H), |
| | | | calcd for | 7.33-7.26 (m, 3H), 6.98-6.90 (m, 3H), 5.17 (dq, J = 9.1, |
| | | | C₂₈H₃₇N₂O₅, | 6.3 Hz, 1H), 4.79 (ddd, J = 8.6, 5.3, 3.4 Hz, |
| | | | 481.2697; | 1H), 4.13 (t, J = 9.0 Hz, 1H), 2.24 (ddt, J = 11.5, |
| | | | found, | 7.7, 3.7 Hz, 1H), 2.16-2.04 (m, 1H), |
| | | | 481.2699 | 1.90 (dt, J = 14.1, 7.3 Hz, 1H), |
| | | | | 1.75-1.59 (m, 6H), 1.51 (ddt, J = 25.7, 15.3, 5.8 Hz, |
| | | | | 6H), 1.34 (dd, J = 13.6, 6.3 Hz, 2H), 1.29 (d, J = 6.3 Hz, |
| | | | | 3H), 1.19 (ddd, J = 13.7, 9.3, 5.0 Hz, |
| | | | | 1H), 1.06-0.93 (m, 2H) |
| | | | | ¹³C NMR (101 MHz, CDCl₃) δ 171.92, |
| | | | | 168.41, 159.59, 157.76, 139.81, 131.22, |
| | | | | 129.59, 128.78, 126.03, 120.88, 115.41, |
| | | | | 82.45, 75.57, 51.63, 42.06, 38.69, 37.56, |
| | | | | 33.56, 32.11, 27.41, 27.13, 25.10, 25.04, |
| | | | | 24.58, 21.79, 18.73 |
| 121 | — | — | HRMS-ESI | ¹H NMR (400 MHz, CDCl₃) δ 12.14 (d, J = 0.5 Hz, |
| | | | (m/z) | 1H), 8.71 (d, J = 7.5 Hz, 1H), 8.02 (d, |
| | | | [M + H]⁺ | J = 5.2 Hz, 1H), 7.35-7.27 (m, 2H), |
| | | | calcd for | 7.01-6.90 (m, 3H), 6.88 (d, J = 5.2 Hz, 1H), |
| | | | C₂₉H₃₉N₂O₆, | 5.16 (dq, J = 9.2, 6.3 Hz, 1H), 4.84-4.74 (m, 1H), |
| | | | 511.2803; | 4.12 (t, J = 9.0 Hz, 1H), 3.95 (s, 3H), |
| | | | found, | 2.24 (dq, J = 10.8, 3.5 Hz, 1H), 2.18-2.04 (m, |
| | | | 511.2806 | 1H), 1.98-1.83 (m, 1H), 1.66 (ddt, J = 19.5, |
| | | | | 8.2, 5.2 Hz, 6H), 1.57-1.41 (m, 6H), 1.33 (d, |
| | | | | J = 6.1 Hz, 2H), 1.29 (d, J = 6.3 Hz, 3H), |
| | | | | 1.19 (ddd, J = 13.6, 9.4, 5.0 Hz, 1H), |
| | | | | 1.06-0.94 (m, 2H) |
| | | | | ¹³C NMR (101 MHz, CDCl₃) δ 171.90, |
| | | | | 168.74, 159.60, 155.36, 148.71, 140.59, |
| | | | | 130.54, 129.59, 120.87, 115.41, 109.44, |
| | | | | 99.98, 82.44, 75.56, 56.08, 51.67, 42.06, |
| | | | | 38.68, 37.56, 33.57, 32.11, 27.38, 27.13, |
| | | | | 25.10, 25.04, 24.57, 21.81, 18.73 |
| 122 | — | — | HRMS-ESI | ¹H NMR (400 MHz, CDCl₃) δ 12.15 (s, 1H), |
| | | | (m/z) | 8.69 (d, J = 7.5 Hz, 1H), 8.01 (d, J = 5.2 Hz, |
| | | | [M + H]⁺ | 1H), 6.87 (d, J = 5.2 Hz, 1H), 6.02-5.80 (m, |
| | | | calcd for | 1H), 5.29 (dq, J = 17.2, 1.6 Hz, 1H), 5.17 (dd, |
| | | | C₂₆H₃₉N₂O₆, | J = 10.4, 1.6 Hz, 1H), 4.99 (dq, J = 9.1, 6.3 Hz, |
| | | | 475.2803; | 1H), 4.79-4.69 (m, 1H), 4.17-4.02 (m, |
| | | | found, | 2H), 3.95 (s, 3H), 3.02 (t, J = 9.1 Hz, 1H), |
| | | | 475.2818 | 2.20 (dq, J = 10.9, 3.6 Hz, 1H), |
| | | | | 2.14-2.01 (m, 1H), 2.01-1.83 (m, 1H), 1.83-1.64 (m, |
| | | | | 2H), 1.64-1.47 (m, 10H), 1.40 (d, J = 6.3 Hz, |
| | | | | 3H), 1.29-1.18 (m, 3H), 1.15-1.00 (m, |
| | | | | 2H) |
| | | | | ¹³C NMR (101 MHz, CDCl₃) δ 171.94, |
| | | | | 168.71, 155.33, 148.68, 140.56, 134.30, |
| | | | | 130.57, 116.92, 109.41, 85.44, 75.73, 73.89, |
| | | | | 56.07, 51.61, 42.11, 38.62, 37.41, 33.85, |
| | | | | 32.06, 27.33, 27.14, 25.13, 25.06, 24.63, |
| | | | | 21.97, 18.52 |
| 123 | — | — | HRMS-ESI | ¹H NMR (400 MHz, CDCl₃) δ 12.13 (d, J = 0.5 Hz, |
| | | | (m/z) | 1H), 8.68 (d, J = 7.5 Hz, 1H), 8.01 (d, |
| | | | [M + H]⁺ | J = 5.2 Hz, 1H), 6.88 (d, J = 5.2 Hz, 1H), |
| | | | calcd for | 5.06 (dq, J = 9.4, 6.3 Hz, 1H), 4.83 (t, J = 9.4 Hz, |
| | | | C₂₇H₄₁N₂O₇, | 1H), 4.81-4.74 (m, 1H), 3.95 (s, 3H), |
| | | | 505.2908; | 2.58 (hept, J = 7.0 Hz, 1H), 2.26 (dq, J = 11.2, 3.9 Hz, |
| | | | found, | 1H), 2.13-2.02 (m, 1H), 1.84 (dq, J = 15.9, |
| | | | 505.2912 | 8.0 Hz, 1H), 1.79-1.60 (m, 5H), |
| | | | | 1.59-1.46 (m, 7H), 1.30 (td, J = 9.1, 8.4, 3.5 Hz, |
| | | | | 2H), 1.24 (d, J = 6.3 Hz, 3H), 1.20 (dd, J = 7.0, |
| | | | | 1.4 Hz, 6H), 1.16 (dd, J = 9.4, 4.4 Hz, |
| | | | | 1H), 1.09-0.94 (m, 2H) |
| | | | | ¹³C NMR (101 MHz, CDCl₃) δ 176.23, |
| | | | | 172.00, 168.70, 155.37, 148.72, 140.55, |
| | | | | 130.54, 109.44, 74.10, 56.08, 51.47, 40.84, |
| | | | | 38.43, 37.17, 34.32, 33.69, 32.10, 27.09, |
| | | | | 26.96, 25.09, 25.02, 24.50, 21.90, 19.07, |
| | | | | 19.02, 17.98 |
| 124 | — | — | HRMS-ESI | ¹H NMR (400 MHz, CDCl₃) δ 12.16 (d, J = 0.5 Hz, |
| | | | (m/z) | 1H), 8.69 (d, J = 7.4 Hz, 1H), 8.01 (d, |
| | | | [M + H]⁺ | J = 5.2 Hz, 1H), 6.87 (d, J = 5.2 Hz, 1H), |
| | | | calcd for | 4.96 (dq, J = 9.2, 6.3 Hz, 1H), 4.77-4.69 (m, 1H), |
| | | | C₂₆H₄₁N₂O₆, | 3.94 (s, 3H), 3.50 (ddt, J = 23.2, 8.6, 6.7 Hz, |
| | | | 477.2959; | 2H), 2.93 (t, J = 9.2 Hz, 1H), 2.28-2.13 (m, |
| | | | found, | 1H), 2.13-1.99 (m, 1H), 2.00-1.83 (m, |
| | | | 477.2975 | 1H), 1.72 (ddt, J = 29.4, 12.2, 5.9 Hz, 2H), |
| | | | | 1.65-1.47 (m, 12H), 1.39 (d, J = 6.3 Hz, |
| | | | | 3H), 1.21 (tt, J = 10.0, 4.7 Hz, 3H), 1.07 (dtd, |
| | | | | J = 15.2, 11.5, 7.5 Hz, 2H), 0.94 (t, J = 7.4 Hz, |
| | | | | 3H) |
| | | | | ¹³C NMR (101 MHz, CDCl₃) δ 171.97, |
| | | | | 168.70, 155.33, 148.68, 140.55, 130.58, |
| | | | | 109.40, 85.24, 75.94, 74.70, 56.06, 51.60, |
| | | | | 42.20, 38.47, 37.39, 33.90, 32.07, 27.32, |
| | | | | 27.13, 25.15, 25.08, 24.66, 23.39, 21.99, |
| | | | | 18.43, 10.75 |
| 125 | — | — | HRMS-ESI | ¹H NMR (400 MHz, CDCl₃) δ 12.15 (s, 1H), |
| | | | (m/z) | 8.69 (d, J = 7.4 Hz, 1H), 8.01 (d, J = 5.2 Hz, |
| | | | [M + H]⁺ | 1H), 6.87 (d, J = 5.2 Hz, 1H), 4.95 (dq, J = 9.1, |
| | | | calcd for | 6.3 Hz, 1H), 4.82-4.61 (m, 1H), 3.95 (s, |
| | | | C₂₄H₃₇N₂O₆, | 3H), 3.45 (s, 3H), 2.87 (t, J = 9.2 Hz, 1H), |
| | | | 449.2646; | 2.27-2.15 (m, 1H), 2.13-2.00 (m, 1H), |
| | | | found, | 1.87 (dd, J = 13.9, 6.6 Hz, 1H), 1.83-1.65 (m, |
| | | | 449.2639 | 2H), 1.60 (dt, J = 10.1, 3.1 Hz, 3H), 1.52 (dd, |
| | | | | J = 14.1, 4.5 Hz, 5H), 1.45-1.35 (m, 4H), |
| | | | | 1.25 (ddd, J = 21.6, 10.9, 5.8 Hz, 3H), |
| | | | | 1.08 (ddd, J = 20.9, 10.3, 6.1 Hz, 2H) |
| | | | | ¹³C NMR (101 MHz, CDCl₃) δ 172.00, |
| | | | | 168.70, 155.33, 148.68, 140.56, 130.57, |
| | | | | 109.41, 87.09, 75.59, 60.44, 56.07, 51.52, |
| | | | | 42.21, 38.47, 37.33, 33.89, 32.07, 25.17, |
| | | | | 25.09, 22.11, 18.40 |
| 126 | — | (Neat) | HRMS-ESI | ¹H NMR (400 MHz, CDCl₃) δ 12.03 (d, J = 0.6 Hz, |
| | | 3373, | (m/z) | 1H), 8.83 (d, J = 8.3 Hz, 1H), 8.04 (d, |
| | | 2934, | [M]⁺ | J = 5.2 Hz, 1H), 7.37-7.09 (m, 7H), |
| | | 1746, | calcd for | 7.03-6.84 (m, 4H), 5.43 (dq, J = 9.1, 6.3 Hz, 1H), |
| | | 1650, | C₂₉H₃₂N₂O₇, | 4.95 (ddd, J = 8.3, 3.7, 1.2 Hz, 1H), 4.13 (t, J = 7.2 Hz, |
| | | 1529, | 520.221; | 1H), 3.94 (s, 3H), 2.11-1.98 (m, |
| | | 1491, | found, | 1H), 3.90 (d, J = 3.8 Hz, 1H), 3.83 (dd, J = 10.5, |
| | | 1238, | 520.2208 | 1.2 Hz, 1H), 3.23-3.09 (m, 2H), |
| | | 1210 | | 2.75 (td, J = 11.8, 2.3 Hz, 1H), 2.34 (dqt, J = 10.1, |
| | | | | 4.9, 2.5 Hz, 1H), 2.20 (dd, J = 13.4, 10.6 Hz, |
| | | | | 1H), 1.52-1.40 (m, 1H), 1.35 (d, J = 6.3 Hz, |
| | | | | 3H) |
| 127 | — | — | HRMS-ESI | ¹H NMR (400 MHz, CDCl₃) δ 12.11 (d, J = 0.5 Hz, |
| | | | (m/z) | 1H), 8.64 (d, J = 7.6 Hz, 1H), 8.00 (d, |
| | | | [M + H]⁺ | J = 5.2 Hz, 1H), 7.29 (d, J = 7.1 Hz, 2H), |
| | | | calcd for | 7.19 (t, J = 7.3 Hz, 1H), 7.16-7.10 (m, 2H), |
| | | | C₂₈H₃₇N₂O₇, | 6.87 (d, J = 5.1 Hz, 1H), 5.13 (dq, J = 9.4, 6.2 Hz, |
| | | | 513.2595; | 1H), 4.98 (t, J = 9.5 Hz, 1H), 4.78 (ddd, J = 7.8, |
| | | | found, | 5.3, 3.4 Hz, 1H), 3.94 (s, 3H), 2.79 (dd, J = 13.6, |
| | | | 513.2596 | 3.8 Hz, 1H), 2.57 (hept, J = 7.0 Hz, |
| | | | | 1H), 2.23 (dd, J = 13.6, 11.1 Hz, 2H), |
| | | | | 2.01 (dq, J = 14.9, 5.1 Hz, 1H), 1.89 (s, 1H), |
| | | | | 1.62-1.50 (m, 3H), 1.41-1.29 (m, 2H), 1.28 (d, J = 6.2 Hz, |
| | | | | 3H), 1.20 (d, J = 7.0 Hz, 6H), |
| | | | | 1.03-0.90 (m, 1H) |
| | | | | ¹³C NMR (101 MHz, CDCl₃) δ 176.27, |
| | | | | 171.98, 168.69, 155.36, 148.71, 140.54, |
| | | | | 139.98, 130.49, 128.98, 128.38, 126.05, |
| | | | | 109.43, 73.92, 56.07, 51.35, 43.50, 38.12, |
| | | | | 34.23, 26.98, 25.92, 24.28, 21.79, 19.04, |
| | | | | 19.00, 18.01 |
| 128 | — | — | HRMS-ESI | ¹H NMR (400 MHz, CDCl₃) δ 12.12 (s, 1H), |
| | | | (m/z) | 8.68 (d, J = 7.5 Hz, 1H), 8.00 (d, J = 5.2 Hz, |
| | | | [M + H]⁺ | 1H), 7.35-7.27 (m, 2H), 7.27-7.21 (m, |
| | | | calcd for | 2H), 7.20-7.08 (m, 3H), 7.02-6.93 (m, |
| | | | C₃₀H₃₅N₂O₆, | 3H), 6.86 (d, J = 5.2 Hz, 1H), 5.22 (dq, J = 9.1, |
| | | | 519.2490; | 6.3 Hz, 1H), 4.83-4.69 (m, 1H), 4.28 (t, |
| | | | found, | J = 9.2 Hz, 1H), 3.93 (s, 3H), 3.13 (dd, J = 13.4, |
| | | | 519.2499 | 3.5 Hz, 1H), 2.28-2.13 (m, 2H), |
| | | | | 2.11-1.96 (m, 2H), 1.64-1.53 (m, 2H), |
| | | | | 1.53-1.40 (m, 2H), 1.34 (d, J = 6.3 Hz, 4H), |
| | | | | 1.08-0.99 (m, 1H) |
| | | | | ¹³C NMR (101 MHz, CDCl₃) δ 171.90, |
| | | | | 168.73, 159.33, 155.36, 148.71, 140.58, |
| | | | | 140.44, 130.51, 129.70, 129.03, 128.27, |
| | | | | 125.88, 121.13, 115.37, 109.46, 81.95, 75.39, |
| | | | | 56.08, 51.48, 45.34, 38.67, 31.60, 27.04, |
| | | | | 26.21, 24.36, 22.67, 21.81, 18.74 |
| 129 | — | — | HRMS-ESI | ¹H NMR (400 MHz, CDCl₃) δ 12.03 (s, 1H), |
| | | | (m/z) | 8.83 (d, J = 8.1 Hz, 1H), 8.04 (d, J = 5.2 Hz, |
| | | | [M + H]⁺ | 1H), 7.31 (t, J = 8.0 Hz, 2H), 7.04 (d, J = 8.5 Hz, |
| | | | calcd for | 2H), 6.98 (t, J = 13 Hz, 1H), 6.93 (d, J = 8.6 Hz, |
| | | | C₃₀H₃₅N₂O₈, | 2H), 6.88 (d, J = 5.3 Hz, 1H), 6.79 (d, |
| | | | 551.2388; | J = 8.6 Hz, 2H), 5.42 (dd, J = 9.0, 6.3 Hz, |
| | | | found, | 1H), 4.94 (dd, J = 8.3, 3.5 Hz, 1H), 4.11 (t, J = 8.3 Hz, |
| | | | 551.2394 | 1H), 3.95 (s, 3H), 3.94-3.89 (m, |
| | | | | 1H), 3.83 (d, J = 10.5 Hz, 1H), 3.76 (s, 3H), |
| | | | | 3.20-3.08 (m, 2H), 2.79 (t, J = 10.9 Hz, 1H), |
| | | | | 2.35-2.21 (m, 1H), 2.21-2.09 (m, 1H), |
| | | | | 2.09-1.96 (m, 1H), 1.47 (s, 1H), 1.34 (d, J = 6.3 Hz, |
| | | | | 3H) |
| | | | | ¹³C NMR (126 MHz, CDCl₃) δ 169.05, |
| | | | | 168.84, 159.19, 157.88, 155.29, 148.73, |
| | | | | 140.67, 132.15, 130.43, 129.99, 129.70, |
| | | | | 121.18, 115.36, 113.80, 109.50, 82.83, 74.47, |
| | | | | 68.58, 67.79, 56.07, 55.19, 53.66, 38.98, |
| | | | | 37.64, 32.26, 18.99 |
| 130 | — | — | HRMS-ESI | ¹H NMR (400 MHz, CDCl₃) δ 12.06 (s, 1H), |
| | | | (m/z) | 8.90 (d, J = 8.2 Hz, 1H), 8.02 (d, J = 5.2 Hz, |
| | | | [M]⁺ | 1H), 7.33-7.22 (m, 2H), 7.00-6.83 (m, |
| | | | calcd for | 4H), 5.37 (dq, J = 9.0, 6.3 Hz, 1H), 4.97 (ddd, |
| | | | C₂₄H₃₀N₂O₇, | J = 8.3, 3.6, 1.2 Hz, 1H), 4.11-3.95 (m, 3H), |
| | | | 458.2053; | 3.94 (s, 3H), 3.75 (td, J = 11.7, 2.3 Hz, 1H), |
| | | | found, | 3.49 (dt, J = 12.0, 3.6 Hz, 1H), 2.01 (ddt, J = 14.6, |
| | | | 458.2059 | 11.4, 3.2 Hz, 1H), 1.94-1.78 (m, 1H), |
| | | | | 1.66 (tt, J = 13.7, 7.4 Hz, 1H), 1.46-1.36 (m, |
| | | | | 1H), 1.33 (d, J = 6.3 Hz, 3H), 1.25-1.08 (m, |
| | | | | 1H), 0.91 (t, J = 7.4 Hz, 3H) |
| | | | | ¹³C NMR (101 MHz, CDCl₃) δ 169.07, |
| | | | | 168.88, 159.29, 155.28, 148.71, 140.69, |
| | | | | 130.43, 129.61, 120.99, 115.20, 109.53, |
| | | | | 82.86, 74.77, 68.81, 67.75, 56.07, 53.75, |
| | | | | 38.58, 32.38, 25.15, 18.96, 12.74 |
| 131 | — | — | HRMS-ESI | ¹H NMR (400 MHz, CDCl₃) δ 12.07 (d, J = 0.6 Hz, |
| | | | (m/z) | 1H), 8.86 (d, J = 8.2 Hz, 1H), 8.01 (d, |
| | | | [M]⁺ | J = 5.2 Hz, 1H), 6.90-6.84 (m, 1H), |
| | | | calcd for | 5.13 (dq, J = 9.0, 6.3 Hz, 1H), 4.91 (dt, J = 8.3, 2.5 Hz, |
| | | | C₂₂H₃₄N₂O₇, | 1H), 3.94 (d, J = 1.5 Hz, 5H), 3.73 (ddd, J = 11.7, |
| | | | 438.2366; | 10.7, 2.6 Hz, 1H), 3.48-3.38 (m, |
| | | | found, | 1H), 3.37 (dd, J = 8.3, 6.2 Hz, 1H), 3.22 (dd, J = 8.4, |
| | | | 438.2366 | 6.6 Hz, 1H), 2.91 (dd, J = 9.0, 7.3 Hz, |
| | | | | 1H), 1.94-1.56 (m, 4H), 1.37 (d, J = 6.4 Hz, |
| | | | | 3H), 1.34-1.11 (m, 2H), 1.00-0.89 (m, 9H) |
| | | | | ¹³C NMR (101 MHz, CDCl₃) δ 169.03, |
| | | | | 168.86, 155.24, 148.67, 140.64, 130.48, |
| | | | | 109.46, 85.67, 80.56, 75.30, 69.40, 68.27, |
| | | | | 56.05, 53.79, 38.63, 33.01, 29.14, 25.04, |
| | | | | 19.54, 19.48, 19.07, 12.84 |
| 132 | — | — | HRMS-ESI | ¹H NMR (500 MHz, CDCl₃) δ 12.02 (s, 1H), |
| | | | (m/z) | 8.84 (d, J = 8.3 Hz, 1H), 8.04 (d, J = 5.2 Hz, |
| | | | [M + H]⁺ | 1H), 7.31 (t, J = 7.8 Hz, 2H), 7.09 (dd, J = 8.1, |
| | | | calcd for | 5.7 Hz, 2H), 7.02-6.78 (m, 6H), |
| | | | C₂₉H₃₂FN₂O₇, | 5.40 (dt, J = 12.6, 6.4 Hz, 1H), 4.95 (dd, J = 8.2, |
| | | | 539.2188; | 3.2 Hz, 1H), 4.12 (t, J = 8.2 Hz, 1H), |
| | | | found, | 3.99-3.90 (m, 4H), 3.86 (d, J = 10.5 Hz, 1H), |
| | | | 539.2197 | 3.26-3.15 (m, 1H), 3.12 (dd, J = 13.7, 5.3 Hz, |
| | | | | 1H), 2.88 (t, J = 11.4 Hz, 1H), 2.31 (d, J = 5.2 Hz, |
| | | | | 1H), 2.24-2.18 (m, 1H), 2.03 (dd, J = 14.3, |
| | | | | 11.6 Hz, 1H), 1.50-1.40 (m, 1H), |
| | | | | 1.34 (d, J = 6.3 Hz, 3H) |
| | | | | ¹³C NMR (126 MHz, CDCl₃) δ 169.05, |
| | | | | 168.88, 161.34 (d, J = 243.9 Hz), 159.05, |
| | | | | 155.30, 148.74, 140.69, 135.94 (d, J = 3.1 Hz), |
| | | | | 130.40, 130.35 (d, J = 7.8 Hz), 115.26, |
| | | | | 115.14 (d, J = 21.1 Hz), 109.52, 82.60, 74.40, |
| | | | | 68.46, 68.03, 56.08, 53.65, 38.74, 37.63, |
| | | | | 32.39, 30.94, 18.91 |
| 133 | — | — | HRMS-ESI | ¹H NMR (500 MHz, CDCl₃) δ 12.03 (s, 1H), |
| | | | (m/z) | 8.79 (d, J = 8.2 Hz, 1H), 8.02 (d, J = 5.2 Hz, |
| | | | [M]⁺ | 1H), 7.19-7.11 (m, 2H), 7.01-6.90 (m, |
| | | | calcd for | 2H), 6.86 (d, J = 5.2 Hz, 1H), 5.22 (dq, J = 9.2, |
| | | | C₂₇H₃₃FN₂O₇, | 6.3 Hz, 1H), 4.88 (ddd, J = 8.3, 3.7, 1.3 Hz, |
| | | | 516.2272; | 1H), 3.94 (s, 3H), 3.87-3.74 (m, 2H), |
| | | | found, | 3.50 (dd, J = 9.8, 6.9 Hz, 1H), 3.38 (dd, J = 9.8, |
| | | | 516.2280 | 6.9 Hz, 1H), 3.30 (dd, J = 13.4, 4.4 Hz, |
| | | | | 1H), 3.07 (dt, J = 12.0, 3.8 Hz, 1H), 3.01 (dd, |
| | | | | J = 9.2, 7.6 Hz, 1H), 2.75-2.64 (m, 1H), |
| | | | | 2.25-2.14 (m, 1H), 2.09-1.98 (m, 1H), |
| | | | | 1.87 (ddt, J = 14.5, 11.1, 3.3 Hz, 1H), 1.41 (d, J = 6.3 Hz, |
| | | | | 3H), 1.35-1.27 (m, 1H), |
| | | | | 1.16-1.04 (m, 1H), 0.63-0.51 (m, 2H), 0.28-0.17 (m, |
| | | | | 2H) |
| | | | | ¹⁹F NMR (471 MHz, CDCl₃) δ −105.43, |
| | | | | −141.28 (m) |
| 134 | — | — | HRMS-ESI | ¹H NMR (400 MHz, CDCl₃) δ 12.01 (d, J = 0.5 Hz, |
| | | | (m/z) | 1H), 8.80 (d, J = 8.4 Hz, 1H), 8.03 (d, |
| | | | [M + H]⁺ | J = 5.2 Hz, 1H), 7.19-7.07 (m, 2H), |
| | | | calcd for | 7.07-6.92 (m, 2H), 6.88 (d, J = 5.1 Hz, 1H), |
| | | | C₂₇H₃₄FN₂O₈, | 5.34 (dq, J = 9.4, 6.3 Hz, 1H), 4.95 (ddd, J = 8.4, |
| | | | 533.2294; | 3.5, 1.2 Hz, 1H), 4.80 (dd, J = 9.4, 7.2 Hz, |
| | | | found, | 1H), 3.95 (s, 3H), 3.93-3.88 (m, 1H), |
| | | | 533.2302 | 3.86 (dd, J = 10.6, 1.2 Hz, 1H), 3.16 (dt, J = 11.9, |
| | | | | 4.0 Hz, 1H), 2.95-2.80 (m, 2H), 2.55 (hept, J = 7.0 Hz, |
| | | | | 1H), 2.27-2.09 (m, 2H), |
| | | | | 2.04-1.90 (m, 1H), 1.44-1.33 (m, 1H), 1.29 (d, J = 6.3 Hz, |
| | | | | 3H), 1.20 (d, J = 2.5 Hz, 3H), |
| | | | | 1.18 (d, J = 2.6 Hz, 3H) |
| | | | | ¹³C NMR (101 MHz, CDCl₃) δ 176.49, |
| | | | | 169.02, 168.99, 161.40 (d, J = 244.4 Hz), |
| | | | | 155.29, 148.73, 140.66, 135.32 (d, J = 3.2 Hz), |
| | | | | 130.37 (d, J = 7.6 Hz), 115.25 (d, J = 21.1 Hz), |
| | | | | 109.54, 76.69, 73.01, 68.70, 68.50, |
| | | | | 56.06, 53.66, 36.88, 36.82, 34.09, 31.67, |
| | | | | 19.08, 18.83, 18.49 |
| 135 | — | — | HRMS-ESI | ¹H NMR (500 MHz, CDCl₃) δ 12.05 (d, J = 0.5 Hz, |
| | | | (m/z) | 1H), 8.88 (d, J = 8.3 Hz, 1H), |
| | | | [M]⁺ | 8.11-8.05 (m, 2H), 8.03 (d, J = 5.2 Hz, 1H), |
| | | | calcd for | 7.65-7.55 (m, 1H), 7.52-7.42 (m, 2H), 6.88 (d, J = 5.2 Hz, |
| | | | C₂₅H₃₀N₂O₈, | 1H), 5.45 (dq, J = 9.2, 6.3 Hz, 1H), |
| | | | 486.2002; | 5.06-4.95 (m, 2H), 4.08-3.98 (m, 2H), |
| | | | found, | 3.95 (s, 3H), 3.79 (ddd, J = 12.4, 10.0, 2.6 Hz, 1H), |
| | | | 486.2004 | 3.52 (ddd, J = 11.9, 4.9, 3.2 Hz, 1H), |
| | | | | 2.04-1.88 (m, 2H), 1.60-1.39 (m, 2H), 1.35 (d, J = 6.3 Hz, |
| | | | | 3H), 1.28-1.18 (m, 1H), 0.90 (t, J = 7.4 Hz, |
| | | | | 3H) |
| | | | | ¹³C NMR (101 MHz, CDCl₃) δ 169.05, |
| | | | | 169.03, 165.96, 155.30, 148.74, 140.67, |
| | | | | 133.36, 130.42, 129.74, 129.60, 128.58, |
| | | | | 109.52, 77.45, 73.58, 69.22, 68.60, 56.07, |
| | | | | 53.80, 37.13, 31.60, 24.52, 18.60, 12.05 |
| 136 | — | — | HRMS-ESI | ¹H NMR (500 MHz, CDCl₃) δ 12.03 (s, 1H), |
| | | | (m/z) | 8.85 (d, J = 8.3 Hz, 1H), 8.02 (d, J = 5.2 Hz, |
| | | | [M]⁺ | 1H), 6.87 (d, J = 5.2 Hz, 1H), 5.30-5.22 (m, |
| | | | calcd for | 1H), 4.96 (ddd, J = 8.5, 3.3, 1.7 Hz, 1H), |
| | | | C₂₂H₃₀N₂O₈, | 4.75 (dd, J = 9.2, 7.7 Hz, 1H), 4.02-3.95 (m, 2H), |
| | | | 450.2002; | 3.95 (s, 3H), 3.73 (ddd, J = 11.8, 10.5, 2.6 Hz, |
| | | | found, | 1H), 3.46 (ddd, J = 11.9, 4.7, 3.4 Hz, 1H), |
| | | | 450.2007 | 1.85 (dddt, J = 46.8, 14.0, 10.4, 3.5 Hz, 2H), |
| | | | | 1.70-1.59 (m, 1H), 1.46 (tt, J = 13.3, 7.4 Hz, |
| | | | | 1H), 1.39-1.32 (m, 1H), 1.29 (d, J = 6.3 Hz, |
| | | | | 3H), 1.18 (dp, J = 14.5, 7.4 Hz, 1H), 1.03 (dtt, |
| | | | | J = 6.5, 4.9, 2.9 Hz, 2H), 0.94-0.87 (m, 5H) |
| | | | | ¹³C NMR (126 MHz, CDCl₃) δ 174.40, |
| | | | | 169.04, 168.96, 155.29, 148.72, 140.65, |
| | | | | 130.43, 109.50, 73.55, 69.09, 68.39, 56.07, |
| | | | | 53.76, 36.88, 31.41, 24.30, 18.52, 12.88, |
| | | | | 11.94, 8.58, 8.33 |
| 137 | — | — | ESIMS | — |
| | | | m/z 398.3 | |
| | | | ([M + H]⁺) | |
| 138 | — | — | ESIMS | — |
| | | | m/z 330.3 | |
| | | | ([M + H]⁺) | |
| 139 | — | — | ESIMS | — |
| | | | m/z 350.3 | |
| | | | ([M + H]⁺) | |
| 140 | — | — | ESIMS | — |
| | | | m/z 308.3 | |
| | | | ([M + H]⁺) | |
| 141 | — | 2873, | HRMS-ESI | ¹H NMR (400 MHz, CDCl₃) δ 8.82 (s, 3H), |
| | | 1745, | (m/z) | 4.94-4.81 (m, 1H), 4.23 (s, 1H), 3.63 (dd, J = 8.5, |
| | | 1232, | [M + H]⁺ | 6.4 Hz, 1H), 3.38 (s, 3H), 3.27 (dd, J = 8.5, |
| | | 1095 | calcd for | 6.4 Hz, 1H), 3.12 (t, J = 8.8 Hz, 1H), |
| | | | C₁₅H₃₀NO₄, | 3.03-2.94 (m, 1H), 2.23 (s, 2H), 1.83 (dq, J = 13.1, |
| | | | 288.2175; | 6.7 Hz, 1H), 1.77-1.63 (m, 3H), |
| | | | found, | 1.63-1.49 (m, 1H), 1.42-1.30 (m, 1H), 1.37 (d, J = 6.2 Hz, |
| | | | 288.2199 | 3H), 1.29-1.17 (m, 1H), 0.91 (d, J = 6.8 Hz, |
| | | | | 6H) |
| 142 | — | — | ESIMS | — |
| | | | m/z 350.3 | |
| | | | ([M + H]⁺) | |
| 143 | — | — | ESIMS | — |
| | | | m/z 392.3 | |
| | | | ([M + H]⁺) | |
| 144 | — | — | ESIMS | — |
| | | | m/z 364.4 | |
| | | | ([M + H]⁺) | |
| 145 | — | — | ESIMS | — |
| | | | m/z 270.3 | |
| | | | ([M + H]⁺) | |
| 146 | — | — | ESIMS | — |
| | | | m/z 336.4 | |
| | | | ([M + H]⁺) | |
| 147 | — | (Neat) | — | ¹H NMR (400 MHz, CDCl₃) δ 8.82 (s, 3H), |
| | | 2878, | | 4.90-4.78 (m, 1H), 4.24 (s, 1H), 3.55 (s, |
| | | 1744, | | 3H), 3.41 (s, 3H), 3.09-2.93 (m, 2H), |
| | | 1232, | | 2.22 (d, J = 4.8 Hz, 2H), 1.70 (m, 3H), |
| | | 1095 | | 1.62-1.49 (m, 1H), 1.44-1.32 (m, 1H), 1.37 (d, J = 6.3 Hz, |
| | | | | 3H), 1.28-1.14 (m, 1H) |
| | | | | ¹³C NMR (101 MHz, CDCl₃) δ 169.38, 86.05, |
| | | | | 84.46, 73.44, 61.24, 58.23, 52.77, 28.20, |
| | | | | 25.10, 22.53, 21.34, 18.01 |
| 148 | — | (Neat) | HRMS-ESI | ¹H NMR (400 MHz, CDCl₃) δ 8.89 (s, 3H), |
| | | 2932, | (m/z) | 7.31-7.21 (m, 2H), 7.05-6.97 (m, 2H), |
| | | 2859, | [M + H]⁺ | 6.97-6.91 (m, 1H), 5.12 (dq, J = 9.2, 6.2 Hz, 1H), |
| | | 1740, | calcd for | 4.30 (t, J = 4.2 Hz, 1H), 4.24 (t, J = 8.7 Hz, |
| | | 1492, | C₁₉H₃₀NO₄, | 1H), 3.37 (qt, J = 9.1, 6.6 Hz, 2H), |
| | | 1230 | 336.2175; | 3.30-3.24 (m, 1H), 2.38-2.19 (m, 2H), 1.92-1.47 (m, |
| | | | found, | 6H), 1.41-1.22 (m, 5H), 0.69 (t, J = 7.4 Hz, |
| | | | 336.2205 | 3H) |
| 149 | — | (Neat) | HRMS-ESI | ¹H NMR (400 MHz, CDCl₃) δ 8.61 (s, 3H), |
| | | 2955, | (m/z) | 7.35-7.21 (m, 2H), 7.21-7.05 (m, 3H), |
| | | 2872, | [M]⁺ | 5.15 (dt, J = 12.2, 6.4 Hz, 1H), 4.46 (s, 1H), |
| | | 1744, | calcd for | 4.13 (d, J = 11.7 Hz, 1H), 3.81-3.64 (m, 1H), |
| | | 1233, | C₂₀H₃₁NO₄, | 3.45 (dd, J = 8.4, 6.2 Hz, 1H), 3.33-3.18 (m, |
| | | 1074 | 349.2253; | 2H), 3.03-2.88 (m, 2H), 2.58 (t, J = 11.2 Hz, |
| | | | found, | 1H), 2.18 (dd, J = 13.2, 11.2 Hz, 1H), |
| | | | 349.2255 | 2.01-1.91 (m, 1H), 1.85 (dq, J = 13.2, 6.6 Hz, 1H), |
| | | | | 1.75 (t, J = 13.1 Hz, 1H), 1.38 (d, J = 6.1 Hz, |
| | | | | 3H), 1.33-1.18 (m, 1H), 0.93 (dd, J = 6.7, |
| | | | | 3.3 Hz, 6H) |
| 150 | — | — | HRMS-ESI | — |
| | | | (m/z) | |
| | | | [M + H]⁺ | |
| | | | calcd for | |
| | | | C₁₆H₃₀NO₄, | |
| | | | 300.2169; | |
| | | | found, | |
| | | | 300.2173 | |
| 151 | — | — | HRMS-ESI | — |
| | | | (m/z) | |
| | | | [M + H]⁺ | |
| | | | calcd for | |
| | | | C₁₆H₃₀NO₄, | |
| | | | 300.2169; | |
| | | | found, | |
| | | | 300.2174 | |
| 152 | — | — | ESIMS | — |
| | | | m/z 348.4 | |
| | | | ([M + H]⁺) | |
| 153 | — | — | ESIMS | — |
| | | | m/z 314.35 | |
| | | | ([M + H]⁺) | |
| 154 | — | — | ESIMS | — |
| | | | m/z 300.36 | |
| | | | ([M + H]⁺) | |
| 155 | — | — | ESIMS | — |
| | | | m/z 362.3 | |
| | | | ([M + H]⁺) | |
| 156 | — | — | ESIMS | — |
| | | | m/z 372.3 | |
| | | | ([M + H]⁺) | |
| 157 | — | — | HRMS-ESI | — |
| | | | (m/z) | |
| | | | [M + H]⁺ | |
| | | | calcd for | |
| | | | C₁₈H₃₄NO₆, | |
| | | | 360.2381; | |
| | | | found, | |
| | | | 360.2384 | |
| 158 | — | — | ESIMS | — |
| | | | m/z 258 | |
| | | | ([M + H]⁺) | |
| 159 | — | — | ESIMS | — |
| | | | m/z 310.3 | |
| | | | ([M + H]⁺) | |
| 160 | — | — | ESIMS | — |
| | | | m/z 338.3 | |
| | | | ([M + H]⁺) | |
| 161 | — | — | ESIMS | — |
| | | | m/z 338.3 | |
| | | | ([M + H]⁺) | |
| 162 | — | — | ESIMS | — |
| | | | m/z 332.4 | |
| | | | ([M + H]⁺) | |
| 163 | — | — | ESIMS | — |
| | | | m/z 366.3 | |
| | | | ([M + H]⁺) | |
| 164 | — | — | ESIMS | — |
| | | | m/z 352.3 | |
| | | | ([M + H]⁺) | |
| 165 | — | — | ESIMS | — |
| | | | m/z 352.3 | |
| | | | ([M + H]⁺) | |
| 166 | — | — | ESIMS | — |
| | | | m/z 360.4 | |
| | | | ([M + H]⁺) | |
| 167 | — | — | ESIMS | — |
| | | | m/z 324.4 | |
| | | | ([M + H]⁺) | |
| 168 | — | — | ESIMS | — |
| | | | m/z 354.4 | |
| | | | ([M + H]⁺) | |
| 169 | — | — | ESIMS | — |
| | | | m/z 326.4 | |
| | | | ([M + H]⁺) | |
| 170 | — | — | ESIMS | — |
| | | | m/z 298.4 | |
| | | | ([M + H]⁺) | |
| 171 | — | — | ESIMS | — |
| | | | m/z 362.4 | |
| | | | ([M + H]⁺) | |
| 172 | — | — | ESIMS | — |
| | | | m/z 368.3 | |
| | | | ([M + H]⁺) | |
| 173 | — | — | ESIMS | — |
| | | | m/z 308 | |
| | | | ([M + H]⁺) | |
| 174 | — | — | ESIMS | — |
| | | | m/z 400.3 | |
| | | | ([M + H]⁺) | |
| 175 | — | — | ESIMS | — |
| | | | m/z 288 | |
| | | | ([M + H]⁺) | |
| 176 | — | — | ESIMS | — |
| | | | m/z 388.3 | |
| | | | ([M + H]⁺) | |
| 177 | — | — | ESIMS | — |
| | | | m/z 366 | |
| | | | ([M + H]⁺) | |
| 178 | — | — | ESIMS | — |
| | | | m/z 382.3 | |
| | | | ([M + H]⁺) | |
| 179 | — | — | ESIMS | — |
| | | | m/z 336.6 | |
| | | | ([M + H]⁺) | |
| 180 | — | — | ESIMS | — |
| | | | m/z 300.5 | |
| | | | ([M + H]⁺) | |
| 181 | — | — | HRMS-ESI | ¹H NMR (400 MHz, CDCl₃) δ 7.39-7.26 (m, |
| | | | (m/z) | 10H), 5.47 (d, J = 7.9 Hz, 1H), |
| | | | [M + Na]⁺ | 5.36-5.20 (m, 1H), 5.03 (d, J = 11.2 Hz, 1H), 4.91 (d, J = 11.4 Hz, |
| | | | calcd for | 1H), 4.57 (d, J = 11.2 Hz, 1H), |
| | | | C₂₈H₃₇NNaO₇, | 4.51 (d, J = 11.4 Hz, 1H), 4.47 (d, J = 8.1 Hz, |
| | | | 522.2462; | 1H), 3.78 (s, 2H), 3.66-3.50 (m, 2H), |
| | | | found, | 3.37 (d, J = 12.3 Hz, 1H), 3.28 (t, J = 8.9 Hz, 1H), |
| | | | 522.2466 | 2.06 (t, J = 12.5 Hz, 1H), 1.53 (d, J = 6.9 Hz, |
| | | | | 1H), 1.45 (s, 9H), 1.32 (d, J = 6.3 Hz, 3H) |
| | | | | ¹³C NMR (101 MHz, CDCl₃) δ 169.73, |
| | | | | 155.46, 138.79, 138.12, 128.37, 128.31, |
| | | | | 128.06, 127.94, 127.75, 127.56, 84.59, 79.94, |
| | | | | 77.93, 75.20, 75.17, 73.17, 67.36, 66.77, |
| | | | | 55.34, 36.62, 34.22, 28.31, 24.68, 19.00 |
| 182 | — | — | HRMS-ESI | δ 7.36-7.27 (m, 10H), 5.09 (dd, J = 6.1, 2.3 Hz, |
| | | | (m/z) | 1H), 5.06 (dd, J = 8.8, 6.3 Hz, 1H), |
| | | | [M + Na]⁺ | 4.98 (d, J = 11.2 Hz, 1H), 4.86 (d, J = 11.4 Hz, |
| | | | calcd for | 1H), 4.57 (d, J = 11.2 Hz, 1H), 4.53 (d, J = 11.5 Hz, |
| | | | C₃₃H₄₅NNaO₉, | 1H), 4.04-3.96 (m, 1H), 3.88 (dd, J = 12.0, |
| | | | 622.3006; | 6.1 Hz, 1H), 3.79-3.69 (m, 2H), |
| | | | found, | 3.52-3.45 (m, 1H), 3.37 (t, J = 8.4 Hz, 1H), |
| | | | 622.2987 | 1.83-1.72 (m, 1H), 1.49 (m, 19H), 1.37 (d, J = 6.3 Hz, |
| | | | | 3H) |
| | | | | ¹³C NMR (101 MHz, CDCl₃) δ 167.44, |
| | | | | 152.62, 138.98, 138.23, 128.36, 128.32, |
| | | | | 128.06, 127.75, 127.71, 127.47, 83.86, 82.81, |
| | | | | 79.09, 77.22, 75.00, 74.56, 73.64, 69.84, |
| | | | | 68.57, 59.10, 35.27, 27.92, 18.72 |
| 183 | — | — | HRMS-ESI | ¹H NMR (400 MHz, CDCl₃) δ 5.15 (dd, J = 6.6, |
| | | | (m/z) | 1.8 Hz, 1H), 4.97-4.82 (m, 1H), |
| | | | [M + Na]⁺ | 4.03 (dd, J = 11.9, 1.8 Hz, 1H), 3.97-3.83 (m, |
| | | | calcd for | 2H), 3.78 (dt, J = 9.5, 4.8 Hz, 1H), 3.49 (ddd, |
| | | | C₁₉H₃₃NNaO₉, | J = 11.8, 5.5, 3.1 Hz, 1H), 3.42 (td, J = 8.2, |
| | | | 442.2048; | 4.1 Hz, 1H), 3.02 (d, J = 4.2 Hz, 1H), 2.35 (d, |
| | | | found, | J = 4.8 Hz, 1H), 2.09 (td, J = 11.4, 10.9, 4.8 Hz, |
| | | | 442.2049 | 1H), 1.72-1.60 (m, 1H), 1.50 (s, 18H), |
| | | | | 1.44 (d, J = 6.3 Hz, 3H) |
| | | | | ¹³C NMR (101 MHz, CDCl₃) δ 167.34, |
| | | | | 152.70, 82.91, 75.41, 74.65, 70.07, 69.36, |
| | | | | 68.37, 58.92, 35.41, 27.93, 18.78, 17.70 |
| 184 | — | — | HRMS-ESI | ¹H NMR (400 MHz, CDCl₃) δ 7.43-7.27 (m, |
| | | | (m/z) | 5H), 5.52 (d, J = 7.7 Hz, 1H), 5.21 (dq, J = 8.8, |
| | | | [M + Na]⁺ | 6.3 Hz, 1H), 5.00 (d, J = 11.3 Hz, 1H), |
| | | | calcd for | 4.54 (d, J = 11.4 Hz, 1H), 4.50-4.41 (m, |
| | | | C₂₅H₃₉NNaO₇, | 1H), 3.90-3.77 (m, 3H), 3.72 (td, J = 12.4, |
| | | | 488.2623; | 1.9 Hz, 1H), 3.50-3.39 (m, 2H), |
| | | | found, | 3.39-3.30 (m, 1H), 3.20 (t, J = 8.9 Hz, 1H), |
| | | | 488.2619 | 2.11-1.96 (m, 1H), 1.49 (ddd, J = 10.9, 6.7, 3.1 Hz, 3H), |
| | | | | 1.44 (d, J = 1.6 Hz, 9H), 1.41-1.35 (m, 1H), |
| | | | | 1.35-1.30 (m, 1H), 1.29 (d, J = 6.3 Hz, 3H), |
| | | | | 0.86 (t, J = 7.4 Hz, 3H) |
| | | | | ¹³C NMR (101 MHz, CDCl₃) δ 169.71, |
| | | | | 155.47, 138.34, 128.34, 127.92, 127.65, |
| | | | | 84.45, 79.90, 78.08, 75.00, 73.19, 73.05, |
| | | | | 67.42, 67.01, 55.37, 34.29, 32.46, 28.31, |
| | | | | 19.26, 18.98, 13.94 |
| 185 | — | — | HRMS-ESI | ¹H NMR (400 MHz, CDCl₃) δ 7.40-7.27 (m, |
| | | | (m/z) | 5H), 5.08 (dd, J = 6.0, 2.3 Hz, 1H), |
| | | | [M + Na]⁺ | 5.05-4.98 (m, 1H), 4.96 (d, J = 11.4 Hz, 1H), |
| | | | calcd for | 4.54 (d, J = 11.3 Hz, 1H), 4.02 (dd, J = 11.9, 2.3 Hz, |
| | | | C₃₀H₄₇NNaO₉, | 1H), 3.88 (dd, J = 11.9, 6.1 Hz, 1H), |
| | | | 588.3143; | 3.84-3.73 (m, 2H), 3.57-3.41 (m, 3H), |
| | | | found, | 3.33-3.24 (m, 1H), 2.05-1.94 (m, 1H), |
| | | | 588.3156 | 1.76-1.62 (m, 1H), 1.56-1.47 (m, 19H), 1.44 (d, J = 1.8 Hz, |
| | | | | 1H), 1.43-1.35 (m, 1H), 1.33 (d, J = 6.2 Hz, |
| | | | | 3H), 1.31-1.25 (m, 1H), 0.87 (t, J = 7.4 Hz, |
| | | | | 3H) |
| | | | | ¹³C NMR (101 MHz, CDCl₃) δ 167.45, |
| | | | | 152.62, 138.43, 128.32, 127.92, 127.61, |
| | | | | 83.64, 82.79, 78.82, 74.78, 73.61, 72.24, |
| | | | | 69.94, 68.69, 59.13, 35.22, 32.42, 28.31, |
| | | | | 27.98, 27.92, 19.31, 18.68, 13.93 |
| 186 | — | — | HRMS-ESI | ¹H NMR (400 MHz, CDCl₃) δ 5.49 (d, J = 7.3 Hz, |
| | | | (m/z) | 1H), 5.11 (dq, J = 8.4, 6.3 Hz, 1H), |
| | | | [M + Na]⁺ | 4.51 (d, J = 7.5 Hz, 1H), 3.86-3.74 (m, 3H), |
| | | | calcd for | 3.63 (dt, J = 9.0, 6.5 Hz, 1H), 3.52-3.42 (m, 2H), |
| | | | C₁₈H₃₃NNaO₇, | 3.42-3.38 (m, 1H), 3.38-3.24 (m, 2H), |
| | | | 398.2149; | 2.14-1.96 (m, 1H), 1.67-1.48 (m, 3H), 1.45 (s, |
| | | | found, | 9H), 1.41 (d, J = 6.3 Hz, 3H), 1.39-1.30 (m, |
| | | | 398.2157 | 2H), 0.91 (t, J = 7.3 Hz, 3H) |
| | | | | ¹³C NMR (101 MHz, CDCl₃) δ 169.71, |
| | | | | 155.44, 79.99, 75.12, 74.88, 71.53, 68.27, |
| | | | | 68.13, 55.16, 33.64, 32.04, 28.31, 19.27, |
| | | | | 18.92, 13.83 |
| 187 | — | (Neat) | — | ¹H NMR (400 MHz, CDCl₃) δ 8.20-8.00 (m, |
| | | 2958, | | 2H), 7.96-7.77 (m, 2H), 7.71-7.38 (m, |
| | | 2933, | | 6H), 7.13 (d, J = 7.5 Hz, 1H), 5.58 (dq, J = 8.9, |
| | | 2871, | | 6.4 Hz, 1H), 5.22-5.00 (m, 2H), 3.98 (d, |
| | | 1732, | | J = 2.4 Hz, 2H), 3.79 (td, J = 12.0, 1.9 Hz, |
| | | 1659, | | 1H), 3.61-3.34 (m, 4H), 2.25 (ddt, J = 15.1, |
| | | 1518, | | 11.9, 3.0 Hz, 1H), 1.68 (ddt, J = 15.5, 5.6, 2.3 Hz, |
| | | 1485, | | 1H), 1.41 (d, J = 6.4 Hz, 3H), |
| | | 1274, | | 1.33-1.03 (m, 4H), 0.67 (t, J = 7.3 Hz, 3H) |
| | | 1200, | | ¹³C NMR (101 MHz, CDCl₃) δ 169.84, |
| | | 1161, | | 167.08, 165.52, 133.72, 133.20, 131.87, |
| | | 1136, | | 129.82, 129.74, 128.59, 128.44, 127.19, |
| | | 1087, | | 76.51, 76.11, 73.05, 72.25, 67.66, 67.44, |
| | | 1070, | | 54.54, 33.70, 32.02, 19.00, 18.72, 13.72 |
| | | 1028, | | |
| | | 708, | | |
| | | 557 | | |
| 188 | — | — | HRMS-ESI | ¹H NMR (400 MHz, CDCl₃) δ 5.16 (dd, J = 6.9, |
| | | | (m/z) | 1.9 Hz, 1H), 4.92 (dq, J = 8.7, 6.3 Hz, |
| | | | [M + Na]⁺ | 1H), 4.05 (dd, J = 12.0, 1.9 Hz, 1H), |
| | | | calcd for | 3.90-3.81 (m, 2H), 3.65-3.56 (m, 2H), |
| | | | C₂₃H₄₁NNaO₉, | 3.54-3.44 (m, 3H), 3.44-3.36 (m, 1H), 1.85 (dt, J = 8.0, |
| | | | 498.2682; | 5.3 Hz, 2H), 1.48 (d, J = 1.7 Hz, 20H), |
| | | | found, | 1.42 (d, J = 6.3 Hz, 3H), 1.36 (dd, J = 15.1, |
| | | | 498.2674 | 7.4 Hz, 2H), 0.91 (t, J = 7.4 Hz, 3H) |
| | | | | ¹³C NMR (101 MHz, CDCl₃) δ 167.47, |
| | | | | 152.68, 82.85, 75.24, 74.31, 70.92, 70.32, |
| | | | | 69.36, 58.88, 34.03, 31.94, 27.94, 19.27, |
| | | | | 18.41, 13.82 |
| 189 | — | — | ESIMS | ¹H NMR (400 MHz, CDCl₃) δ 7.39-7.27 (m, |
| | | | m/z 398.3 | 10H), 5.25 (d, J = 7.2 Hz, 1H), |
| | | | ([M—Boc + H]⁺) | 5.04-4.89 (m, 2H), 4.69-4.53 (m, 3H), 4.34 (s, 1H), |
| | | | | 3.43 (t, J = 8.8 Hz, 1H), 3.35 (s, 1H), |
| | | | | 2.08-1.95 (m, 1H), 1.97-1.73 (m, 2H), |
| | | | | 1.60-1.47 (m, 4H), 1.45 (s, 9H), 1.37 (d, J = 6.3 Hz, |
| | | | | 3H), 1.35-1.24 (m, 1H) |
| | | | | ¹³C NMR (150 MHz, CDCl₃) δ 172.7, 155.1, |
| | | | | 138.6, 138.3, 128.3, 128.3, 128.0, 127.9, |
| | | | | 127.7, 127.5, 84.1, 82.2, 79.8, 75.7, 72.6, |
| | | | | 72.5, 53.0, 28.8, 28.3, 27.3, 22.4, 21.3, 18.40 |
| 190 | — | — | HRMS-ESI | ¹H NMR (400 MHz, CDCl₃) δ 5.08 (dd, J = 6.0, |
| | | | (m/z) | 2.4 Hz, 1H), 4.94 (dq, J = 8.7, 6.3 Hz, |
| | | | [M + Na]⁺ | 1H), 4.01 (dd, J = 11.9, 2.4 Hz, 1H), 3.88 (dd, |
| | | | calcd for | J = 11.9, 6.1 Hz, 1H), 3.75 (dtd, J = 14.6, 6.0, |
| | | | C₂₇H₄₉NNaO₉, | 5.2, 3.2 Hz, 3H), 3.53-3.35 (m, 3H), |
| | | | 554.3300; | 3.17-3.06 (m, 2H), 2.02-1.90 (m, 1H), 1.83 (dq, J = 13.3, |
| | | | found, | 6.8, 6.1 Hz, 1H), 1.50 (s, 21H), |
| | | | 554.3315. | 1.36 (d, J = 6.3 Hz, 3H), 1.35-1.28 (m, 2H), |
| | | | | 0.94-0.87 (m, 9H) |
| | | | | ¹³C NMR (101 MHz, CDCl₃) δ 167.49, |
| | | | | 152.63, 84.72, 82.76, 80.27, 78.68, 73.79, |
| | | | | 72.12, 69.98, 68.73, 59.18, 44.51, 35.15, |
| | | | | 32.40, 29.07, 27.99, 27.93, 19.57, 19.41, |
| | | | | 19.32, 18.67, 13.97 |
| 191 | — | — | HRMS-ESI | ¹H NMR (400 MHz, CDCl₃) δ 5.56 (d, J = 8.1 Hz, |
| | | | (m/z) | 1H), 5.34 (dq, J = 9.0, 6.4 Hz, 1H), |
| | | | [M + Na]⁺ | 4.76 (t, J = 8.8 Hz, 1H), 4.52 (d, J = 8.1 Hz, 1H), |
| | | | calcd for | 3.87-3.78 (m, 2H), 3.74 (td, J = 12.0, 2.0 Hz, |
| | | | C₂₂H₃₇NNaO₈, | 1H), 3.61 (dt, J = 9.1, 6.7 Hz, 1H), |
| | | | 466.2411; | 3.48-3.37 (m, 2H), 3.33 (ddd, J = 8.5, 6.4, 2.1 Hz, |
| | | | found, | 1H), 2.19-1.98 (m, 1H), 1.64 (ddd, J = 12.7, |
| | | | 466.2418 | 8.1, 4.6 Hz, 1H), 1.60-1.49 (m, 2H), |
| | | | | 1.49-1.41 (m, 10H), 1.38-1.27 (m, 5H), |
| | | | | 1.10-1.01 (m, 2H), 0.94-0.85 (m, 5H) |
| | | | | ¹³C NMR (101 MHz, CDCl₃) δ 174.12, |
| | | | | 169.92, 155.54, 80.05, 76.23, 75.69, 73.02, |
| | | | | 72.02, 67.87, 67.37, 55.42, 33.79, 32.28, |
| | | | | 28.39, 19.32, 18.78, 14.02, 12.87, 8.86, 8.60 |
| 192 | — | — | HRMS-ESI | ¹H NMR (400 MHz, CDCl₃) δ 5.57 (d, J = 7.9 Hz, |
| | | | (m/z) | 1H), 5.32 (dq, J = 8.9, 6.4 Hz, 1H), |
| | | | [M + H]⁺ | 4.75 (t, J = 8.7 Hz, 1H), 4.53 (d, J = 8.1 Hz, 1H), |
| | | | calcd for | 3.88-3.80 (m, 2H), 3.75 (td, J = 11.9, 2.2 Hz, |
| | | | C₂₂H₄₀NO₈, | 1H), 3.56 (dt, J = 9.1, 6.6 Hz, 1H), |
| | | | 446.2748; | 3.48-3.29 (m, 3H), 2.59 (hept, J = 7.0 Hz, 1H), |
| | | | found, | 2.19-2.04 (m, 1H), 1.65-1.54 (m, 1H), |
| | | | 446.2773 | 1.51-1.38 (m, 11H), 1.37-1.30 (m, 1H), |
| | | | | 1.30-1.26 (m, 4H), 1.21 (t, J = 7.0 Hz, 6H), 0.88 (t, |
| | | | | J = 7.3 Hz, 3H) |
| | | | | ¹³C NMR (101 MHz, CDCl₃) δ 176.05, |
| | | | | 169.91, 155.53, 80.04, 75.75, 75.51, 72.74, |
| | | | | 72.08, 68.10, 67.51, 55.40, 34.10, 33.88, |
| | | | | 32.22, 28.39, 19.31, 19.27, 18.68, 13.98 |
| 193 | — | — | HRMS-ESI | ¹H NMR (400 MHz, CDCl₃) δ 8.19-7.99 (m, |
| | | | (m/z) | 2H), 7.64-7.52 (m, 1H), 7.46 (td, J = 7.6, 4.5 Hz, |
| | | | [M + H]⁺ | 2H), 5.73 (d, J = 8.1 Hz, 1H), |
| | | | calcd for | 5.62-5.42 (m, 1H), 5.05 (t, J = 8.7 Hz, 1H), 4.61 (dd, J = 8.1, |
| | | | C₂₅H₃₈NO₈, | 2.3 Hz, 1H), 3.98-3.70 (m, 3H), |
| | | | 480.2592; | 3.61-3.32 (m, 4H), 2.32-2.10 (m, 1H), |
| | | | found, | 1.79-1.57 (m, 1H), 1.54-1.43 (m, 9H), 1.38 (d, J = 6.4 Hz, |
| | | | 480.2604 | 3H), 1.34-0.97 (m, 4H), 0.66 (t, J = 7.3 Hz, |
| | | | | 3H) |
| | | | | ¹³C NMR (101 MHz, CDCl₃) δ 169.92, |
| | | | | 165.54, 155.58, 133.48, 133.18, 130.11, |
| | | | | 129.82, 129.71, 128.43, 128.41, 80.04, 76.65, |
| | | | | 75.87, 73.08, 71.97, 67.85, 67.27, 55.38, |
| | | | | 33.78, 32.00, 28.33, 18.99, 18.75, 13.71 |
| 194 | — | (Neat) | — | ¹H NMR (400 MHz, CDCl₃) δ 5.09 (dd, J = 6.0, |
| | | 3076, | | 2.4 Hz, 1H), 5.03-4.91 (m, 3H), 4.85 (d, |
| | | 2979, | | J = 12.7 Hz, 2H), 4.35 (d, J = 12.0 Hz, 1H), |
| | | 2935, | | 4.21 (d, J = 12.5 Hz, 1H), 4.05-3.97 (m, |
| | | 2872, | | 1H), 3.88 (dt, J = 12.0, 3.2 Hz, 3H), |
| | | 1759, | | 3.79 (ddd, J = 11.9, 9.4, 2.9 Hz, 1H), |
| | | 1708, | | 3.59-3.45 (m, 2H), 3.22 (t, J = 8.2 Hz, 1H), |
| | | 1456, | | 2.05-1.93 (m, 1H), 1.74 (s, 3H), 1.71 (s, 4H), 1.38 (d, J = 6.3 Hz, |
| | | 1367, | | 3H) |
| | | 1317, | | ¹³C NMR (101 MHz, CDCl₃) δ 167.51, |
| | | 1248, | | 152.65, 142.77, 142.34, 112.15, 111.72, |
| | | 1145, | | 83.94, 82.81, 78.71, 76.87, 76.22, 73.66, |
| | | 1123 | | 70.05, 68.74, 59.19, 35.26, 27.98, 19.83, |
| | | | | 19.71, 18.74 |
| 195 | — | — | — | — |
| 196 | — | — | ESIMS | ¹H NMR (400 MHz, CDCl₃) δ 5.00 (d, J = 6.2 Hz, |
| | | | m/z 494.4 | 2H), 4.89 (s, 1H), 4.71 (dq, J = 13.7, 6.2 Hz, |
| | | | ([M + Na]⁺) | 1H), 4.17-4.01 (m, 2H), 3.63-3.49 (m, |
| | | | | 1H), 3.15 (t, J = 8.8 Hz, 1H), 2.62 (s, 1H), |
| | | | | 2.08 (dd, J = 15.3, 5.3 Hz, 1H), |
| | | | | 2.03-1.90 (m, 1H), 1.80 (dd, J = 13.6, 5.5 Hz, 1H), |
| | | | | 1.75 (s, 3H), 1.57 (d, J = 10.7 Hz, 4H), |
| | | | | 1.55-1.49 (m, 18H), 1.49-1.45 (m, 1H), 1.42 (d, J = 6.2 Hz, |
| | | | | 3H) |
| | | | | ¹³C NMR (151 MHz, CDCl₃) δ 169.36, |
| | | | | 152.93, 141.78, 112.18, 85.69, 82.64, 73.41, |
| | | | | 73.09, 58.29, 30.68, 28.45, 27.99, 24.00, |
| | | | | 23.93, 19.68, 18.39 |
| 197 | — | — | ESIMS | ¹H NMR (400 MHz, CDCl₃) δ 5.01-4.93 (m, |
| | | | m/z 548.4 | 2H), 4.87-4.80 (m, 3H), 4.77 (q, J = 6.6 Hz, |
| | | | ([M + Na]⁺) | 1H), 4.21 (d, J = 11.9 Hz, 1H), |
| | | | | 3.99-3.89 (m, 3H), 3.51-3.42 (m, 1H), 3.34 (t, J = 7.2 Hz, |
| | | | | 1H), 2.29-2.12 (m, 1H), 2.04-1.88 (m, |
| | | | | 1H), 1.86-1.75 (m, 1H), 1.73 (s, 6H), 1.65 (t, |
| | | | | J = 9.5 Hz, 4H), 1.59-1.52 (m, 1H), 1.50 (s, |
| | | | | 18H), 1.42 (d, J = 6.4 Hz, 3H) |
| | | | | ¹³C NMR (101 MHz, CDCl₃) δ 169.89, |
| | | | | 152.65, 142.70, 142.36, 111.98, 111.67, |
| | | | | 82.74, 82.64, 81.58, 76.46, 73.99, 73.07, |
| | | | | 58.79, 29.26, 27.99, 27.02, 25.47, 22.20, |
| | | | | 19.74, 19.64, 17.55 |
| 198 | — | — | ESIMS | ¹H NMR (400 MHz, CDCl₃) δ 4.80 (dd, J = 7.1, |
| | | | m/z 552.4 | 3.1 Hz, 1H), 4.77-4.66 (m, 1H), |
| | | | ([M + Na]⁺) | 3.56 (dd, J = 8.5, 6.3 Hz, 1H), 3.39-3.32 (m, 1H), |
| | | | | 3.32-3.16 (m, 4H), 2.33-2.13 (m, 1H), |
| | | | | 1.99-1.89 (m, 1H), 1.78 (ddq, J = 18.8, 12.4, 6.8, |
| | | | | 6.4 Hz, 3H), 1.65 (s, 3H), 1.59 (d, J = 9.3 Hz, |
| | | | | 1H), 1.51 (s, 18H), 1.49-1.43 (m, 1H), |
| | | | | 1.40 (d, J = 6.4 Hz, 3H), 0.93-0.84 (m, 12H) |
| | | | | ¹³C NMR (101 MHz, CDCl₃) δ 169.99, |
| | | | | 152.64, 83.12, 82.56, 82.31, 79.67, 73.34, |
| | | | | 60.34, 58.77, 29.31, 28.97, 28.93, 27.96, |
| | | | | 27.38, 26.64, 25.54, 22.00, 19.52, 19.49, |
| | | | | 19.46, 19.37, 17.53 |
| 199 | — | (Neat) | HRMS-ESI | ¹H NMR (400 MHz, CDCl₃) δ 5.02 (t, J = 4.7 Hz, |
| | | 3530, | (m/z) | 1H), 4.65 (dq, J = 9.4, 6.1 Hz, 1H), |
| | | 2978, | [M + Na]⁺ | 3.51-3.41 (m, 1H), 3.39 (s, 3H), 3.14 (d, J = 0.9 Hz, |
| | | 2936, | calcd for | 1H), 2.95 (ddd, J = 8.4, 6.2, 2.0 Hz, 1H), |
| | | 1740, | C₂₁H₃₇NO₈Na, | 2.27-2.12 (m, 1H), 1.97-1.70 (m, 3H), |
| | | 1701, | 454.2411; | 1.70-1.59 (m, 1H), 1.55-1.46 (m, 1H), 1.51 (s, |
| | | 1359, | found, | 18H), 1.43 (d, J = 6.1 Hz, 3H), 1.34-1.27 (m, |
| | | 1142 | 454.2418 | 2H) |
| 200 | — | (Neat) | HRMS-ESI | ¹H NMR (400 MHz, CDCl₃) δ 4.82 (dd, J = 6.9, |
| | | 2978, | (m/z) | 3.3 Hz, 1H), 4.71 (dq, J = 7.7, 6.3 Hz, |
| | | 2933, | [M + Na]⁺ | 1H), 3.51 (s, 3H), 3.41 (s, 3H), 3.26 (ddd, J = 7.8, |
| | | 2826, | calcd for | 5.9, 2.0 Hz, 1H), 3.15 (t, J = 7.7 Hz, 1H), |
| | | 1741, | C₂₂H₃₉NO₈Na, | 2.25-2.12 (m, 1H), 2.03-1.92 (m, 1H), |
| | | 1702, | 468.2568; | 1.82-1.60 (m, 4H), 1.51 (s, 18H), 1.40 (d, J = 6.3 Hz, |
| | | 1366, | found, | 3H), 1.34-1.23 (m, 2H) |
| | | 1104 | 468.2577 | |
| 201 | — | — | ESIMS | ¹H NMR (400 MHz, CDCl₃) δ 4.96 (s, 1H), |
| | | | m/z 508.4 | 4.86 (s, 1H), 4.82 (dd, J = 6.9, 3.3 Hz, 1H), |
| | | | ([M + Na]⁺) | 4.74 (q, J = 6.8, 6.3 Hz, 1H), 4.19 (d, J = 11.9 Hz, |
| | | | | 1H), 3.95 (d, J = 11.8 Hz, 1H), 3.39 (s, |
| | | | | 3H), 3.36-3.27 (m, 2H), 2.28-2.12 (m, |
| | | | | 1H), 2.07-1.91 (m, 1H), 1.84-1.71 (m, |
| | | | | 4H), 1.70-1.63 (m, 2H), 1.58 (d, J = 1.1 Hz, |
| | | | | 3H), 1.51 (s, 18H), 1.41 (d, J = 6.4 Hz, 3H) |
| | | | | ¹³C NMR (101 MHz, CDCl₃) δ 169.94, |
| | | | | 152.72, 142.45, 111.99, 84.33, 82.85, 82.66, |
| | | | | 72.98, 58.72, 57.85, 29.27, 27.99, 26.76, |
| | | | | 25.34, 22.07, 19.75, 17.62 |
| 202 | — | — | ESIMS | ¹H NMR (400 MHz, CDCl₃) δ 7.32-7.20 (m, |
| | | | m/z 530.4 | 2H), 7.05-6.91 (m, 3H), 4.94 (p, J = 6.4 Hz, |
| | | | ([M + Na]⁺) | 1H), 4.86 (dd, J = 6.9, 3.3 Hz, 1H), 4.31 (t, J = 7.3 Hz, |
| | | | | 1H), 3.49-3.43 (m, 1H), 3.33 (s, |
| | | | | 3H), 2.25 (dd, J = 15.0, 6.6 Hz, 1H), 1.99 (dd, |
| | | | | J = 15.6, 3.5 Hz, 1H), 1.87 (dd, J = 13.4, 6.4 Hz, |
| | | | | 1H), 1.72 (d, J = 10.6 Hz, 3H), 1.55 (s, |
| | | | | 2H), 1.51 (s, 18H), 1.40 (d, J = 6.4 Hz, 3H) |
| | | | | ¹³C NMR (101 MHz, CDCl₃) δ 169.83, |
| | | | | 159.02, 152.65, 129.40, 121.24, 116.16, |
| | | | | 82.97, 82.74, 81.54, 73.03, 58.77, 58.20, |
| | | | | 34.67, 29.24, 28.01, 26.62, 25.41, 22.05, |
| | | | | 17.57 |
| 203 | — | — | ESIMS | ¹H NMR (400 MHz, CDCl₃) δ 7.32-7.19 (m, |
| | | | m/z 570.4 | 2H), 7.04-6.88 (m, 3H), 4.95 (p, J = 6.5 Hz, |
| | | | ([M + Na]⁺) | 1H), 4.91-4.81 (m, 2H), 4.78 (s, 1H), 4.36 (t, |
| | | | | J = 7.1 Hz, 1H), 3.93 (d, J = 12.3 Hz, 1H), |
| | | | | 3.84 (d, J = 12.3 Hz, 1H), 3.69-3.58 (m, |
| | | | | 1H), 2.38-2.21 (m, 1H), 1.98 (dd, J = 15.3, |
| | | | | 3.3 Hz, 1H), 1.86 (dt, J = 14.2, 6.3 Hz, 1H), |
| | | | | 1.82-1.63 (m, 4H), 1.60 (s, 3H), |
| | | | | 1.55-1.53 (m, 1H), 1.51 (s, 18H), 1.41 (d, J = 6.4 Hz, |
| | | | | 3H) |
| | | | | ¹³C NMR (101 MHz, CDCl₃) δ 169.84, |
| | | | | 158.84, 152.60, 142.45, 129.36, 121.14, |
| | | | | 116.03, 111.88, 82.75, 81.23, 80.33, 74.14, |
| | | | | 73.10, 58.79, 29.24, 28.01, 26.70, 25.54, |
| | | | | 22.10, 19.48, 17.53 |
| 204 | — | — | ESIMS | ¹H NMR (400 MHz, CDCl₃) δ 7.32-7.22 (m, |
| | | | m/z 570.4 | 2H), 7.00-6.86 (m, 3H), 4.91-4.82 (m, |
| | | | ([M + Na]⁺) | 3H), 4.80 (s, 1H), 4.43-4.36 (m, 1H), |
| | | | | 4.19 (d, J = 11.6 Hz, 1H), 3.99 (d, J = 11.7 Hz, |
| | | | | 1H), 3.55 (t, J = 7.4 Hz, 1H), 2.15 (dd, J = 11.5, |
| | | | | 4.9 Hz, 1H), 2.10-1.98 (m, 1H), |
| | | | | 1.92 (dt, J = 14.8, 7.5 Hz, 1H), 1.85-1.66 (m, |
| | | | | 3H), 1.65 (s, 3H), 1.63-1.58 (m, 1H), |
| | | | | 1.51 (s, 18H), 1.48 (d, J = 6.4 Hz, 3H), 1.42 (dd, J = 7.6, |
| | | | | 3.6 Hz, 1H) |
| | | | | ¹³C NMR (101 MHz, CDCl₃) δ 169.88, |
| | | | | 157.83, 152.72, 142.19, 129.44, 120.74, |
| | | | | 115.77, 112.39, 82.70, 82.46, 80.58, 72.70, |
| | | | | 58.44, 28.95, 28.00, 26.99, 25.20, 21.81, |
| | | | | 19.72, 17.74 |
| 205 | — | — | ESIMS | ¹H NMR (400 MHz, CDCl₃) δ 7.31-7.21 (m, |
| | | | m/z 572.4 | 2H), 6.97-6.84 (m, 3H), 4.93-4.77 (m, |
| | | | ([M + Na]⁺) | 2H), 4.34 (td, J = 7.1, 1.8 Hz, 1H), 3.54 (dd, J = 8.5, |
| | | | | 6.4 Hz, 1H), 3.46 (t, J = 7.7 Hz, 1H), |
| | | | | 3.32 (dd, J = 8.5, 6.5 Hz, 1H), 2.21-2.08 (m, |
| | | | | 1H), 2.04-1.99 (m, 1H), 1.96-1.83 (m, |
| | | | | 1H), 1.81-1.66 (m, 3H), 1.63-1.54 (m, |
| | | | | 2H), 1.51 (s, 18H), 1.47 (d, J = 6.3 Hz, 3H), |
| | | | | 1.43-1.39 (m, 1H), 0.81 (d, J = 6.7 Hz, 3H), |
| | | | | 0.78 (d, J = 6.7 Hz, 3H) |
| 206 | — | — | ESIMS | ¹H NMR (400 MHz, CDCl₃) δ 8.12-8.02 (m, |
| | | | m/z 520.4 | 2H), 7.63-7.54 (m, 1H), 7.51-7.43 (m, |
| | | | ([M − H]⁻) | 2H), 5.18 (t, J = 8.6 Hz, 1H), 5.00 (td, J = 6.4, |
| | | | | 2.5 Hz, 2H), 3.82 (ddd, J = 8.4, 5.5, 2.9 Hz, |
| | | | | 1H), 2.22-2.06 (m, 2H), 2.02-1.94 (m, |
| | | | | 1H), 1.82-1.57 (m, 6H), 1.52 (s, 18H), |
| | | | | 1.38 (d, J = 6.3 Hz, 3H) |
| | | | | ¹³C NMR (151 MHz, CDCl₃) δ 169.58, |
| | | | | 166.25, 152.90, 133.45, 129.87, 129.65, |
| | | | | 129.50, 128.54, 82.73, 77.95, 72.90, 71.69, |
| | | | | 58.23, 34.68, 31.17, 28.73, 28.00, 25.29, |
| | | | | 24.28, 22.85, 17.92 |
| 207 | — | — | ESIMS | ¹H NMR (400 MHz, CDCl₃) δ 8.63 (d, J = 4.2 Hz, |
| | | | m/z 520.4 | 1H), 8.09-7.97 (m, 2H), 7.58 (tt, J = 6.9, |
| | | | ([M − H]⁻) | 1.3 Hz, 1H), 7.52-7.41 (m, 2H), |
| | | | | 5.12-4.93 (m, 2H), 4.80 (dq, J = 8.8, 6.2 Hz, 1H), |
| | | | | 3.84 (t, J = 8.7 Hz, 1H), 2.20-2.06 (m, 2H), |
| | | | | 2.04-1.95 (m, 1H), 1.89-1.76 (m, 2H), |
| | | | | 1.74-1.64 (m, 1H), 1.63-1.56 (m, 1H), 1.52 (s, |
| | | | | 18H), 1.51-1.47 (m, 4H) |
| | | | | ¹³C NMR (101 MHz, CDCl₃) δ 169.59, |
| | | | | 166.64, 152.88, 149.59, 133.23, 130.03, |
| | | | | 129.64, 128.46, 82.77, 78.63, 75.25, 73.51, |
| | | | | 57.96, 28.55, 28.42, 28.00, 24.31, 22.84, |
| | | | | 18.00 |
| 208 | — | — | ESIMS | ¹H NMR (400 MHz, CDCl₃) δ 8.05 (d, J = 7.2 Hz, |
| | | | m/z 612.4 | 2H), 7.57 (t, J = 7.4 Hz, 1H), 7.45 (t, J = 7.7 Hz, |
| | | | ([M + Na]⁺) | 2H), 5.62-5.49 (m, 1H), |
| | | | | 5.41-5.32 (m, 1H), 5.29-5.19 (m, 1H), 4.91-4.78 (m, |
| | | | | 2H), 4.05 (d, J = 5.2 Hz, 1H), 3.55 (t, J = 7.4 Hz, |
| | | | | 1H), 2.22 (d, J = 6.1 Hz, 1H), 1.94 (dq, J = 14.3, |
| | | | | 7.4, 7.0 Hz, 4H), 1.84-1.62 (m, 4H), |
| | | | | 1.62-1.56 (m, 5H), 1.52 (s, 18H), 1.47 (d, J = 6.4 Hz, |
| | | | | 3H) |
| | | | | ¹³C NMR (101 MHz, CDCl₃) δ 169.67, |
| | | | | 165.60, 152.58, 136.53, 132.87, 130.47, |
| | | | | 129.53, 128.38, 124.79, 82.77, 81.02, 76.10, |
| | | | | 73.45, 73.24, 58.62, 29.17, 27.99, 27.58, |
| | | | | 25.25, 25.10, 22.23, 17.65, 13.11 |
| 209 | — | — | ESIMS | ¹H NMR (400 MHz, CDCl₃) δ 5.79 (dt, J = 15.4, |
| | | | m/z 508.4 | 6.2 Hz, 1H), 5.62-5.50 (m, 1H), |
| | | | ([M + Na]⁺) | 4.99 (t, J = 5.0 Hz, 1H), 4.66 (dq, J = 9.2, 6.2 Hz, |
| | | | | 1H), 4.19-4.08 (m, 2H), 3.55-3.44 (m, |
| | | | | 1H), 3.14 (t, J = 9.0 Hz, 1H), 2.55 (s, 1H), |
| | | | | 2.14-2.02 (m, 3H), 2.02-1.89 (m, 1H), |
| | | | | 1.88-1.75 (m, 1H), 1.75-1.60 (m, 3H), |
| | | | | 1.57-1.53 (m, 2H), 1.51 (s, 18H), 1.42 (d, J = 6.2 Hz, |
| | | | | 3H), 1.01 (t, J = 7.4 Hz, 3H) |
| | | | | ¹³C NMR (101 MHz, CDCl₃) δ 169.35, |
| | | | | 152.90, 137.25, 124.57, 84.94, 82.60, 74.41, |
| | | | | 73.24, 73.21, 58.24, 30.62, 28.41, 27.96, |
| | | | | 25.24, 23.99, 23.84, 18.35, 13.20 |
| 210 | — | (Neat) | HRMS-ESI | ¹H NMR (400 MHz, CDCl₃) δ 4.81 (dd, J = 6.9, |
| | | 2974, | (m/z) | 3.4 Hz, 1H), 4.77-4.65 (m, 1H), |
| | | 1735, | [M + Na]⁺ | 3.53 (dd, J = 8.6, 6.5 Hz, 1H), 3.39 (s, 3H), |
| | | 1686, | calcd for | 3.29 (dd, J = 8.6, 6.5 Hz, 1H), 3.25-3.15 (m, 2H), |
| | | 1366, | C₂₅H₄₅NO₈Na, | 2.25-2.12 (m, 1H), 2.05-1.92 (m, 1H), |
| | | 1106 | 510.3037; | 1.87-1.71 (m, 2H), 1.70-1.63 (m, 2H), |
| | | | found, | 1.62-1.57 (m, 1H), 1.51 (s, 18H), 1.44 (d, J = 10.2 Hz, |
| | | | 510.3050 | 1H), 1.40 (d, J = 6.3 Hz, 3H), |
| | | | | 1.33-1.23 (m, 1H), 0.90 (dd, J = 6.7, 0.8 Hz, 6H) |
| 211 | — | — | ESIMS | ¹H NMR (400 MHz, CDCl₃) δ 7.25 (dt, J = 8.6, |
| | | | m/z 512.4 | 6.9 Hz, 2H), 7.01-6.84 (m, 3H), 4.95 (p, |
| | | | ([M + Na]⁺) | J = 6.4 Hz, 1H), 4.86 (dd, J = 7.0, 2.9 Hz, |
| | | | | 1H), 4.34 (t, J = 6.9 Hz, 1H), 3.56 (t, J = 5.9 Hz, |
| | | | | 1H), 3.26 (dd, J = 8.8, 6.5 Hz, 1H), |
| | | | | 3.15 (dd, J = 8.8, 6.3 Hz, 1H), 2.30 (dt, J = 15.4, |
| | | | | 7.4 Hz, 1H), 2.01-1.92 (m, 1H), 1.85 (dt, J = 14.0, |
| | | | | 6.6 Hz, 1H), 1.79-1.60 (m, 5H), |
| | | | | 1.59-1.54 (m, 1H), 1.51 (s, 18H), 1.42 (d, J = 6.4 Hz, |
| | | | | 3H), 0.79 (d, J = 6.7 Hz, 3H), 0.75 (d, J = 6.7 Hz, |
| | | | | 3H) |
| | | | | ¹³C NMR (101 MHz, CDCl₃) δ 169.82, |
| | | | | 158.85, 152.54, 129.27, 121.04, 116.03, |
| | | | | 82.66, 81.10, 77.25, 73.10, 58.84, 29.27, |
| | | | | 28.79, 27.98, 26.51, 25.66, 21.99, 19.34, |
| | | | | 19.24, 17.44, 14.18 |
| 212 | — | — | ESIMS | ¹H NMR (400 MHz, CDCl₃) δ 8.11-7.98 (m, |
| | | | m/z 614.4 | 2H), 7.56 (tt, J = 6.8, 1.2 Hz, 1H), |
| | | | ([M + Na]⁺) | 7.50-7.39 (m, 2H), 5.30-5.19 (m, 1H), 5.00-4.75 (m, |
| | | | | 2H), 3.57 (q, J = 6.5 Hz, 2H), 3.48 (t, J = 7.4 Hz, |
| | | | | 1H), 2.32-2.13 (m, 1H), 2.04-1.91 (m, |
| | | | | 3H), 1.88-1.59 (m, 4H), 1.52 (s, 18H), |
| | | | | 1.47 (d, J = 6.4 Hz, 3H), 1.44-1.35 (m, 2H), |
| | | | | 1.20-1.06 (m, 4H), 0.75 (t, J = 7.0 Hz, 3H) |
| | | | | ¹³C NMR (101 MHz, CDCl₃) δ 169.64, |
| | | | | 165.60, 152.57, 132.86, 130.44, 129.48, |
| | | | | 128.45, 128.35, 82.70, 82.04, 76.06, 73.26, |
| | | | | 73.05, 58.57, 29.70, 29.12, 28.11, 27.99, |
| | | | | 27.96, 25.18, 22.35, 17.57, 13.85 |
| 213 | — | — | ESIMS | ¹H NMR (400 MHz, CDCl₃) δ 7.34-7.10 (m, |
| | | | m/z 592.4 | 4H), 7.07-6.84 (m, 4H), 6.78 (d, J = 8.4 Hz, |
| | | | ([M + Na]⁺) | 2H), 5.05 (p, J = 6.2 Hz, 1H), 4.93 (dd, J = 6.4, |
| | | | | 2.9 Hz, 1H), 4.66-4.42 (m, 2H), |
| | | | | 2.30-2.14 (m, 1H), 2.14-1.95 (m, 2H), |
| | | | | 1.94-1.70 (m, 3H), 1.66-1.54 (m, 2H), 1.52 (s, 18H), |
| | | | | 1.49 (d, J = 6.4 Hz, 3H) |
| | | | | ¹³C NMR (101 MHz, CDCl₃) δ 169.71, |
| | | | | 158.79, 157.77, 152.67, 129.40, 129.36, |
| | | | | 121.42, 120.91, 116.31, 115.72, 82.78, 81.05, |
| | | | | 79.52, 72.79, 58.46, 34.68, 28.89, 28.03, |
| | | | | 26.93, 25.30, 25.23, 21.90, 17.76 |
| 214 | — | (Neat) | HRMS-ESI | ¹H NMR (400 MHz, CDCl₃) δ 7.36-7.26 (m, |
| | | 3540, | (m/z) | 2H), 7.10-6.94 (m, 3H), 5.01 (t, J = 4.9 Hz, |
| | | 2978, | [M + Na]⁺ | 1H), 4.88 (dq, J = 8.9, 6.2 Hz, 1H), 4.20 (t, J = 8.8 Hz, |
| | | 2935, | calcd for | 1H), 3.84-3.73 (m, 1H), 2.27 (d, J = 2.0 Hz, |
| | | 1741, | C₂₆H₃₉NO₈Na, | 1H), 2.22-1.86 (m, 3H), |
| | | 1701, | 516.2568; | 1.85-1.57 (m, 4H), 1.54-1.45 (m, 19H), 1.32 (d, J = 6.3 Hz, |
| | | 1366, | found, | 3H) |
| | | 1142 | 516.2572 | |
| 215 | — | — | — | ¹H NMR (400 MHz, CDCl₃) δ 7.34-7.18 (m, |
| | | | | 2H), 6.99-6.80 (m, 3H), 4.87 (dd, J = 6.5, |
| | | | | 3.5 Hz, 1H), 4.81 (dd, J = 8.0, 6.4 Hz, 1H), |
| | | | | 4.41-4.26 (m, 1H), 3.73 (dt, J = 8.8, 6.6 Hz, |
| | | | | 1H), 3.55 (dt, J = 8.8, 6.6 Hz, 1H), 3.47 (t, J = 7.8 Hz, |
| | | | | 1H), 2.21-2.09 (m, 1H), |
| | | | | 2.09-1.98 (m, 1H), 1.96-1.85 (m, 1H), 1.83-1.68 (m, |
| | | | | 2H), 1.65-1.53 (m, 3H), 1.51 (d, J = 1.7 Hz, |
| | | | | 18H), 1.47 (d, J = 6.3 Hz, 3H), |
| | | | | 1.45-1.39 (m, 3H), 1.24-1.17 (m, 3H), 0.83-0.78 (m, |
| | | | | 3H) |
| | | | | ¹³C NMR (101 MHz, CDCl₃) δ 169.91, |
| | | | | 158.07, 152.76, 129.38, 120.63, 115.76, |
| | | | | 83.00, 82.66, 80.74, 73.30, 72.87, 58.36, |
| | | | | 34.67, 29.84, 28.86, 28.23, 27.99, 27.19, |
| | | | | 25.62, 25.28, 25.02, 22.47, 21.92, 17.74, |
| | | | | 14.12, 13.97 |
| 216 | — | — | ESIMS | ¹H NMR (400 MHz, CDCl₃) δ 6.05-5.80 (m, |
| | | | m/z 480.4 | 1H), 5.30 (dd, J = 17.2, 1.5 Hz, 1H), |
| | | | ([M + Na]⁺) | 5.25-5.15 (m, 1H), 4.99 (t, J = 4.9 Hz, 1H), |
| | | | | 4.68 (dq, J = 8.9, 6.2 Hz, 1H), 4.35-4.09 (m, 2H), |
| | | | | 3.58-3.43 (m, 1H), 3.16 (t, J = 8.9 Hz, 1H), |
| | | | | 2.61 (s, 1H), 2.15-2.03 (m, 1H), |
| | | | | 2.03-1.90 (m, 1H), 1.87-1.76 (m, 1H), 1.76-1.53 (m, |
| | | | | 5H), 1.51 (s, 18H), 1.42 (d, J = 6.2 Hz, 3H) |
| | | | | ¹³C NMR (101 MHz, CDCl₃) δ 169.32, |
| | | | | 152.86, 134.10, 117.41, 85.16, 82.56, 74.37, |
| | | | | 73.28, 73.07, 58.23, 30.61, 28.40, 27.94, |
| | | | | 23.96, 23.79, 18.28 |
| 217 | — | — | ESIMS | ¹H NMR (400 MHz, CDCl₃) δ 5.01 (dt, J = 9.8, |
| | | | m/z 482.4 | 4.9 Hz, 1H), 4.66 (ddd, J = 9.4, 7.1, 4.0 Hz, |
| | | | ([M + Na]⁺) | 1H), 3.62 (ddt, J = 8.7, 6.6, 2.1 Hz, 2H), |
| | | | | 3.54-3.41 (m, 1H), 3.07 (t, J = 8.9 Hz, 1H), |
| | | | | 2.60 (s, 1H), 2.14-2.03 (m, 1H), |
| | | | | 2.01-1.89 (m, 1H), 1.78 (td, J = 11.9, 10.3, 6.3 Hz, 2H), |
| | | | | 1.69-1.53 (m, 6H), 1.51 (s, 18H), 1.42 (d, J = 6.2 Hz, |
| | | | | 3H), 0.94 (t, J = 7.4 Hz, 3H) |
| | | | | ¹³C NMR (101 MHz, CDCl₃) δ 169.32, |
| | | | | 152.90, 85.42, 82.57, 75.39, 73.27, 73.24, |
| | | | | 58.25, 30.74, 28.40, 27.96, 23.98, 23.95, |
| | | | | 23.39, 18.33, 10.54 |
| 218 | — | — | ESIMS | ¹H NMR (400 MHz, CDCl₃) δ 5.00 (s, 1H), |
| | | | m/z 510.4 | 4.78-4.55 (m, 1H), 4.25-4.05 (m, 1H), |
| | | | ([M + Na]⁺) | 3.65 (t, J = 6.5 Hz, 2H), 3.57-3.44 (m, 1H), |
| | | | | 3.07 (t, J = 8.9 Hz, 1H), 2.61 (s, 1H), |
| | | | | 2.16-2.06 (m, 1H), 2.03-1.91 (m, 1H), 1.88-1.75 (m, |
| | | | | 1H), 1.59 (dd, J = 14.5, 7.7 Hz, 6H), 1.51 (s, |
| | | | | 18H), 1.42 (d, J = 6.2 Hz, 3H), |
| | | | | 1.37-1.30 (m, 3H), 1.29-1.24 (m, 1H), 0.99-0.85 (m, |
| | | | | 3H) |
| | | | | ¹³C NMR (101 MHz, CDCl₃) δ 169.30, |
| | | | | 152.88, 85.45, 82.55, 73.85, 73.25, 73.21, |
| | | | | 58.24, 30.73, 29.88, 28.38, 28.23, 27.94, |
| | | | | 23.97, 23.94, 22.48, 18.32, 13.96 |
| 219 | — | — | ESIMS | ¹H NMR (400 MHz, CDCl₃) δ 7.30-7.21 (m, |
| | | | m/z 558.4 | 2H), 6.94-6.86 (m, 3H), 4.87 (dd, J = 6.5, |
| | | | ([M + Na]⁺) | 3.4 Hz, 1H), 4.85-4.77 (m, 1H), |
| | | | | 4.42-4.27 (m, 1H), 3.71 (dt, J = 8.7, 6.6 Hz, 1H), |
| | | | | 3.57-3.44 (m, 2H), 2.21-2.10 (m, 1H), |
| | | | | 2.10-1.98 (m, 1H), 1.90 (tt, J = 14.0, 6.8 Hz, 1H), |
| | | | | 1.80-1.67 (m, 3H), 1.65-1.55 (m, 2H), 1.51 (s, |
| | | | | 18H), 1.47 (d, J = 6.3 Hz, 3H), |
| | | | | 1.46-1.39 (m, 2H), 0.81 (t, J = 7.4 Hz, 3H) |
| | | | | ¹³C NMR (101 MHz, CDCl₃) δ 169.89, |
| | | | | 158.08, 152.75, 129.39, 120.64, 115.77, |
| | | | | 82.97, 82.64, 80.76, 74.86, 72.87, 58.36, |
| | | | | 31.58, 28.86, 27.99, 27.19, 25.03, 23.33, |
| | | | | 21.93, 17.73, 10.57 |
| 221 | — | (Neat) | HRMS-ESI | ¹H NMR (300 MHz, CDCl₃) δ 7.31 (d, J = 5.0 Hz, |
| | | 2978, | (m/z) | 10H), 4.90-4.75 (m, 3H), |
| | | 2933, | [M + Na]⁺ | 4.68-4.53 (m, 3H), 3.67-3.56 (m, 1H), 3.51 (t, J = 7.6 Hz, |
| | | 1741, | calcd for | 1H), 2.31-2.09 (m, 1H), 2.10-1.75 (m, |
| | | 1701, | C₃₄H₄₇NO₈Na, | 2H), 1.74-1.57 (m, 4H), 1.50 (s, 18H), |
| | | 1365 | 620.3194; | 1.51-1.45 (m, 1H), 1.43 (d, J = 6.4 Hz, 3H) |
| | | | found, | |
| | | | 620.3194 | |
| 222 | — | — | ESIMS | ¹H NMR (400 MHz, CDCl₃) δ 7.26 (s, 2H), |
| | | | m/z 472 | 7.22-7.14 (m, 3H), 5.50 (d, J = 5.8 Hz, 1H), |
| | | | ([M + Na]⁺) | 5.04 (dq, J = 9.5, 6.4 Hz, 1H), 4.29 (d, J = 5.8 Hz, |
| | | | | 1H), 3.67 (t, J = 11.3 Hz, 1H), 3.43 (dd, J = 8.3, |
| | | | | 6.5 Hz, 1H), 3.34-3.19 (m, 4H), |
| | | | | 3.17-3.11 (m, 1H), 3.03 (t, J = 9.3 Hz, 1H), |
| | | | | 2.50-2.39 (m, 1H), 2.14-1.98 (m, 2H), 1.88 (dt, |
| | | | | J = 13.2, 6.7 Hz, 1H), 1.76-1.64 (m, 1H), |
| | | | | 1.43 (s, 9H), 1.39 (d, J = 6.4 Hz, 3H), |
| | | | | 0.95 (dd, J = 6.7, 1.8 Hz, 6H) |
| 223 | — | (Neat) | HRMS-ESI | ¹H NMR (400 MHz, CDCl₃) δ 5.11 (d, J = 7.8 Hz, |
| | | 3357, | (m/z) | 1H), 4.94 (dq, J = 9.3, 6.2 Hz, 1H), |
| | | 2981, | [M + Na]⁺ | 4.45 (d, J = 7.0 Hz, 1H), 3.87-3.76 (m, 1H), |
| | | 2935, | calcd for | 3.56 (dd, J = 9.3, 7.9 Hz, 1H), 2.09-1.95 (m, 1H), |
| | | 2877, | C₁₈H₃₁NO₆Na, | 1.96-1.83 (m, 1H), 1.78-1.52 (m, 4H), |
| | | 1746, | 380.2044; | 1.45 (s, 9H), 1.41 (dd, J = 3.5, 2.7 Hz, 7H), |
| | | 1714 | found, | 1.35 (d, J = 0.8 Hz, 3H), 1.23-1.12 (m, 1H) |
| | | | 380.2028 | |
| 224 | — | (Neat) | HRMS-ESI | ¹H NMR (300 MHz, CDCl₃) δ 5.02 (dd, J = 7.5, |
| | | 2980, | (m/z) | 6.0 Hz, 1H), 4.74 (dq, J = 9.4, 6.1 Hz, |
| | | 2935, | [M + Na]⁺ | 1H), 3.95 (td, J = 8.3, 3.7 Hz, 1H), 3.59 (dd, J = 9.4, |
| | | 1739, | calcd for | 7.8 Hz, 1H), 2.22-2.06 (m, 1H), |
| | | 1703 | C₂₃H₃₉NO₈Na, | 2.04-1.84 (m, 2H), 1.75-1.58 (m, 4H), 1.50 (s, |
| | | | 480.2568; | 18H), 1.45-1.37 (m, 7H), 1.34 (s, 3H) |
| | | | found, | |
| | | | 480.2541 | |
| 225 | — | (Neat) | HRMS-ESI | ¹H NMR (400 MHz, CDCl₃) δ 7.25-7.18 (m, |
| | | 3357, | (m/z) | 4H), 6.89-6.82 (m, 4H), 5.49 (d, J = 6.2 Hz, |
| | | 2934, | [M + Na]⁺ | 1H), 5.03-4.94 (m, 1H), 4.84 (d, J = 10.3 Hz, |
| | | 2867, | calcd for | 1H), 4.58 (s, 2H), 4.53 (d, J = 10.4 Hz, |
| | | 1745, | C₃₀H₄₁NO₉Na, | 1H), 4.30 (s, 1H), 3.88-3.77 (m, 7H), |
| | | 1710, | 582.2674; | 3.61-3.31 (m, 5H), 2.56-2.41 (m, 1H), 1.79 (d, J = 15.1 Hz, |
| | | 1683, | found, | 1H), 1.44 (s, 9H), 1.35 (d, J = 6.3 Hz, |
| | | 1513, | 582.2651 | 3H) |
| | | 1247, | | |
| | | 1082 | | |
| 226 | — | — | — | ¹H NMR (400 MHz, CDCl₃) δ 7.31-7.21 (m, |
| | | | | 2H), 7.01-6.90 (m, 3H), 5.74 (ddt, J = 17.2, |
| | | | | 10.4, 5.5 Hz, 1H), 5.12 (dq, J = 17.2, 1.7 Hz, |
| | | | | 1H), 5.09-5.00 (m, 1H), 4.99-4.90 (m, |
| | | | | 1H), 4.87 (dd, J = 6.7, 3.4 Hz, 1H), 4.34 (t, J = 7.4 Hz, |
| | | | | 1H), 4.02 (ddt, J = 12.8, 5.7, 1.5 Hz, |
| | | | | 1H), 3.95 (ddt, J = 12.8, 5.3, 1.5 Hz, 1H), |
| | | | | 3.61 (td, J = 6.7, 5.8, 2.3 Hz, 1H), 2.30-2.15 (m, |
| | | | | 1H), 1.99 (dd, J = 15.5, 4.0 Hz, 1H), 1.85 (dt, |
| | | | | J = 13.7, 6.5 Hz, 1H), 1.77-1.61 (m, 4H), |
| | | | | 1.54-1.47 (m, 1H), 1.51 (s, 18H), 1.41 (d, J = 6.4 Hz, |
| | | | | 3H) |
| 227 | — | (Neat) | HRMS-ESI | ¹H NMR (400 MHz, CDCl₃) δ 7.30-7.21 (m, |
| | | 2977, | (m/z) | 2H), 7.02-6.90 (m, 3H), 4.98-4.89 (m, |
| | | 2934, | [M + Na]⁺ | 1H), 4.86 (dd, J = 6.9, 3.2 Hz, 1H), 4.32 (t, J = 7.2 Hz, |
| | | 1742, | calcd for | 1H), 3.54 (ddd, J = 7.3, 6.1, 2.3 Hz, |
| | | 1703, | C₂₉H₄₅NO₈Na, | 1H), 3.44 (dt, J = 9.0, 6.6 Hz, 1H), 3.34 (dt, J = 9.0, |
| | | 1366, | 558.3043; | 6.5 Hz, 1H), 2.34-2.19 (m, 1H), |
| | | 1142, | found, | 2.07-1.91 (m, 1H), 1.92-1.78 (m, 1H), |
| | | 1112 | 558.3046 | 1.78-1.61 (m, 4H), 1.51 (s, 18H), 1.44-1.36 (m, |
| | | | | 5H), 1.33-1.23 (m, 1H), 0.77 (t, J = 7.4 Hz, |
| | | | | 3H) |
| 228 | — | (Neat) | HRMS-ESI | ¹H NMR (400 MHz, CDCl₃) δ 5.32 (s, 1H), |
| | | 3436, | (m/z) | 4.86 (dq, J = 9.1, 6.2 Hz, 1H), 4.49-4.33 (m, |
| | | 2939, | [M + Na]⁺ | 1H), 3.38 (td, J = 8.9, 3.0 Hz, 1H), |
| | | 1737, | calcd for | 3.34-3.25 (m, 1H), 2.94 (dd, J = 3.3, 1.2 Hz, 1H), |
| | | 1682, | C₁₅H₂₇NO₆Na, | 2.41-2.33 (m, 1H), 2.17-2.04 (m, 1H), |
| | | 1365, | 340.1731; | 1.96-1.77 (m, 2H), 1.67-1.48 (m, 4H), 1.45 (s, 9H), |
| | | 1165, | found, | 1.40 (d, J = 6.2 Hz, 3H), 1.11-0.97 (m, 1H) |
| | | 1045 | 340.1749 | |
| 229 | — | (Neat) | HRMS-ESI | ¹H NMR (400 MHz, CDCl₃) δ 7.30-7.20 (m, |
| | | 3448, | (m/z) | 2H), 7.21-7.10 (m, 3H), 5.46 (d, J = 7.9 Hz, |
| | | 2957, | [M]⁺ | 1H), 5.16 (dq, J = 9.1, 6.3 Hz, 1H), 4.45 (ddd, |
| | | 1748, | calcd for | J = 8.1, 3.1, 1.5 Hz, 1H), 3.74-3.62 (m, 2H), |
| | | 1713, | C₂₅H₃₉NO₆, | 3.45 (dd, J = 8.4, 6.2 Hz, 1H), 3.34-3.25 (m, |
| | | 1496, | 449.2777; | 1H), 3.25 (dd, J = 8.4, 6.6 Hz, 1H), 3.00 (dt, J = 12.0, |
| | | 1365, | found, | 3.9 Hz, 1H), 2.93 (dd, J = 9.1, 7.6 Hz, |
| | | 1165, | 449.2800 | 1H), 2.59 (td, J = 12.1, 11.7, 2.4 Hz, 1H), |
| | | 1093 | | 2.18 (dd, J = 13.2, 11.4 Hz, 1H), 2.07-1.92 (m, |
| | | | | 1H), 1.94-1.72 (m, 2H), 1.42 (s, 9H), |
| | | | | 1.37 (d, J = 6.3 Hz, 3H), 1.34-1.23 (m, 1H), |
| | | | | 0.93 (dd, J = 6.7, 4.2 Hz, 6H) |
| 230 | — | — | ESIMS | ¹H NMR (400 MHz, CDCl₃) δ 5.28 (s, 1H), |
| | | | m/z 366.3 | 4.82 (dq, J = 9.1, 6.2 Hz, 1H), 4.35 (s, 1H), |
| | | | ([M + Na]⁺) | 3.41-3.21 (m, 1H), 2.09 (s, 1H), 1.90 (dt, J = 15.0, |
| | | | | 5.3 Hz, 1H), 1.60 (dd, J = 10.9, 6.2 Hz, |
| | | | | 3H), 1.50 (d, J = 20.9 Hz, 4H), 1.45 (s, 9H), |
| | | | | 1.38 (d, J = 6.2 Hz, 3H), 1.33-1.14 (m, 4H), |
| | | | | 1.07 (d, J = 14.1 Hz, 1H), 0.90 (t, J = 7.0 Hz, |
| | | | | 3H) |
| | | | | ¹³C NMR (101 MHz, CDCl₃) δ 173.11, |
| | | | | 155.20, 79.74, 75.73, 52.56, 43.95, 34.95, |
| | | | | 28.35, 27.00, 26.76, 24.89, 21.77, 19.47, |
| | | | | 18.75, 14.48 |
| 231 | — | — | ESIMS | ¹H NMR (400 MHz, CDCl₃) δ 5.27 (d, J = 6.5 Hz, |
| | | | m/z 522.4 | 1H), 5.03 (t, J = 6.6 Hz, 1H), 4.34 (s, 1H), |
| | | | ([M + Na]⁺) | 3.66 (t, J = 6.5 Hz, 1H), 2.13-2.00 (m, 1H), |
| | | | | 1.90 (dd, J = 13.5, 6.4 Hz, 1H), 1.58 (ddd, J = 12.4, |
| | | | | 9.4, 5.8 Hz, 2H), 1.48 (s, 5H), 1.45 (s, |
| | | | | 11H), 1.40 (dd, J = 10.1, 2.7 Hz, 4H), 1.34 (d, |
| | | | | J = 6.6 Hz, 3H), 1.32-1.15 (m, 4H), |
| | | | | 1.18-1.05 (m, 22H), 0.88 (t, J = 7.1 Hz, 3H) |
| | | | | ¹³C NMR (101 MHz, CDCl₃) δ 172.39, |
| | | | | 155.23, 79.66, 79.56, 76.80, 53.26, 43.88, |
| | | | | 35.58, 28.33, 27.69, 24.16, 22.16, 21.50, |
| | | | | 18.84, 18.29, 18.20, 18.18, 14.29, 13.43 |
| 232 | — | (Neat) | HRMS-ESI | ¹H NMR (400 MHz, CDCl₃) δ 5.01 (t, J = 4.9 Hz, |
| | | 3549, | (m/z) | 1H), 4.67 (dq, J = 9.1, 6.2 Hz, 1H), |
| | | 2948, | [M + Na]⁺ | 3.55-3.37 (m, 3H), 3.05 (t, J = 8.9 Hz, 1H), 2.56 (d, |
| | | 2878, | calcd for | J = 1.3 Hz, 1H), 2.14-1.74 (m, 4H), |
| | | 1757, | C₂₄H₄₃NO₈Na, | 1.75-1.58 (m, 1H), 1.58-1.42 (m, 4H), 1.51 (s, |
| | | 1727, | 496.2881; | 18H), 1.41 (d, J = 6.2 Hz, 3H), 0.93 (d, J = 6.7 Hz, |
| | | 1688, | found, | 3H), 0.92 (d, J = 6.7 Hz, 3H) |
| | | 1364, | 496.2900 | |
| | | 1091 | | |
| 233 | — | (Neat) | — | ¹H NMR (400 MHz, CDCl₃) δ 7.24-7.13 (m, |
| | | 3435, | | 4H), 6.99-6.84 (m, 4H), 6.72-6.66 (m, |
| | | 2975, | | 2H), 5.32 (d, J = 6.7 Hz, 1H), 5.21 (dq, J = 9.3, |
| | | 1711, | | 6.3 Hz, 1H), 4.47 (q, J = 10.7, 9.7 Hz, |
| | | 1491, | | 2H), 4.25 (t, J = 7.5 Hz, 1H), 2.23-1.89 (m, |
| | | 1166 | | 3H), 1.79-1.59 (m, 2H), 1.57-1.50 (m, |
| | | | | 2H), 1.46 (s, 9H), 1.39 (d, J = 6.3 Hz, 3H), |
| | | | | 1.33-1.14 (m, 1H) |
| | | | | ¹³C NMR (101 MHz, CDCl₃) δ 172.83, |
| | | | | 159.40, 157.84, 155.12, 129.31, 129.23, |
| | | | | 121.33, 120.85, 116.27, 115.70, 82.15, 80.15, |
| | | | | 72.04, 52.56, 28.35, 28.02, 27.05, 22.67, |
| | | | | 21.79, 21.40, 18.45 |
| 234 | — | (Neat) | HRMS-ESI | ¹H NMR (400 MHz, CDCl₃) δ 7.33-7.27 (m, |
| | | 3431, | (m/z) | 2H), 7.09-6.93 (m, 3H), 5.35-5.23 (m, |
| | | 2976, | [M + Na]⁺ | 1H), 5.08 (dq, J = 9.3, 6.3 Hz, 1H), |
| | | 1702, | calcd for | 4.47-4.37 (m, 1H), 4.16 (t, J = 9.0 Hz, 1H), |
| | | 1492, | C₂₁H₃₁NO₆Na, | 3.68-3.55 (m, 1H), 2.35 (t, J = 1.7 Hz, 1H), |
| | | 1365, | 416.2044; | 2.19-2.06 (m, 1H), 1.99-1.86 (m, 2H), |
| | | 1166 | found, | 1.70-1.50 (m, 3H), 1.49-1.35 (m, 1H), 1.45 (s, 9H), |
| | | | 416.2060 | 1.28 (d, J = 6.3 Hz, 3H), 1.23-1.12 (m, 1H) |
| 235 | — | (Neat) | HRMS-ESI | ¹H NMR (400 MHz, CDCl₃) δ 7.33-7.27 (m, |
| | | 3436, | (m/z) | 2H), 7.02-6.96 (m, 1H), 6.89-6.83 (m, |
| | | 2976, | [M + H]⁺ | 2H), 5.32 (d, J = 6.8 Hz, 1H), 4.98 (dq, J = 9.5, |
| | | 1701, | calcd for | 6.2 Hz, 1H), 4.43 (s, 1H), 3.89 (t, J = 8.2 Hz, |
| | | 1491, | C₂₁H₃₁NO₆Na, | 1H), 3.76 (td, J = 8.6, 7.9, 1.1 Hz, 1H), |
| | | 1365, | 416.2044; | 2.95 (d, J = 1.4 Hz, 1H), 2.29-2.16 (m, 1H), |
| | | 1166 | found, | 1.96-1.79 (m, 2H), 1.75-1.56 (m, 3H), |
| | | | 416.2062 | 1.48-1.41 (m, 12H), 1.39-1.29 (m, 2H) |
| 236 | — | (Neat) | HRMS-ESI | ¹H NMR (400 MHz, CDCl₃) δ 6.01-5.93 (m, |
| | | 2980, | (m/z) | 1H), 5.92-5.82 (m, 1H), 4.86-4.74 (m, |
| | | 1739, | [M + Na]⁺ | 2H), 4.74-4.59 (m, 1H), 3.49-3.35 (m, |
| | | 1695, | calcd for | 4H), 3.30-3.15 (m, 1H), 2.54-2.36 (m, |
| | | 1364, | C₂₆H₄₃NO₈Na, | 1H), 2.28-2.03 (m, 3H), 2.02-1.71 (m, |
| | | 1116, | 520.2881; | 4H), 1.70-1.61 (m, 2H), 1.58 (q, J = 3.1 Hz, |
| | | 1055 | found, | 1H), 1.51 (s, 15H), 1.48-1.43 (m, 4H), |
| | | | 520.2903 | 1.43-1.36 (m, 3H) |
| 237 | — | — | HRMS-ESI | — |
| | | | (m/z) | |
| | | | [M + Na]⁺ | |
| | | | calcd for | |
| | | | C₂₅H₄₁NNaO₈, | |
| | | | 506.2724; | |
| | | | found, | |
| | | | 506.2762. | |
| 238 | — | — | HRMS-ESI | — |
| | | | (m/z) | |
| | | | [M + Na]⁺ | |
| | | | calcd for | |
| | | | C₂₅H₄₁NNaO₈, | |
| | | | 506.2724; | |
| | | | found, | |
| | | | 506.2742 | |
| 239 | — | — | ESIMS | ¹H NMR (400 MHz, CDCl₃) δ 5.35-5.21 (m, |
| | | | m/z 420.4 | 1H), 5.01-4.96 (m, 1H), 4.96-4.89 (m, |
| | | | ([M + Na]⁺) | 1H), 4.88-4.83 (m, 1H), 4.34 (s, 1H), |
| | | | | 3.94 (s, 2H), 3.00 (t, J = 9.2 Hz, 1H), 2.05 (d, J = 5.2 Hz, |
| | | | | 1H), 1.97-1.83 (m, 1H), 1.75 (s, |
| | | | | 3H), 1.62 (dtd, J = 14.4, 6.0, 2.8 Hz, 1H), |
| | | | | 1.54-1.46 (m, 3H), 1.45 (s, 9H), 1.43-1.38 (m, |
| | | | | 3H), 1.36 (d, J = 6.3 Hz, 3H), 1.31-1.22 (m, |
| | | | | 1H), 1.21-1.03 (m, 3H), 0.88 (t, J = 7.2 Hz, |
| | | | | 3H) |
| | | | | ¹³C NMR (101 MHz, CDCl₃) δ 172.88, |
| | | | | 155.20, 141.97, 111.90, 85.05, 79.69, 76.40, |
| | | | | 75.38, 52.77, 42.95, 34.51, 28.35, 27.35, |
| | | | | 27.12, 24.94, 21.71, 20.14, 19.85, 18.53, |
| | | | | 14.42 |
| 240 | — | (Neat) | HRMS-ESI | ¹H NMR (400 MHz, CDCl₃) δ 4.94 (t, J = 4.8 Hz, |
| | | 3576, | (m/z) | 1H), 4.58 (dq, J = 9.2, 6.1 Hz, 1H), |
| | | 2932, | [M + Na]⁺ | 3.99 (m, 1H), 3.32 (m, 1H), 3.07-2.99 (m, 2H), |
| | | 1739, | calcd for | 2.11 (m, 1H), 1.83 (m, 1H), 1.78-1.38 (m, |
| | | 1698, | C₂₅H₄₃NO₈Na, | 13H), 1.44 (s, 18H), 1.35 (d, J = 6.2, 3H), |
| | | 1348, | 508.2881; | 1.29-1.17 (m, 1H) |
| | | 1111 | found, | |
| | | | 508.2895 | |
| 241 | — | (Neat) | HRMS-ESI | ¹H NMR (400 MHz, CDCl₃) δ 4.92 (t, J = 5.0 Hz, |
| | | 3530, | (m/z) | 1H), 4.56 (dq, J = 8.8, 6.3 Hz, 1H), |
| | | 2948, | [M + Na]⁺ | 4.08 (qd, J = 5.6, 5.1, 4.0 Hz, 1H), 3.44-3.35 (m, |
| | | 1757, | calcd for | 1H), 3.10 (t, J = 8.7 Hz, 1H), 2.42 (d, J = 1.3 Hz, |
| | | 1748, | C₂₅H₄₃NO₈Na, | 1H), 2.01-1.87 (m, 2H), 1.78-1.55 (m, |
| | | 1696, | 508.2881; | 11H), 1.52-1.45 (m, 3H), 1.43 (s, 18H), |
| | | 1352, | found, | 1.35 (d, J = 6.3 Hz, 3H) |
| | | 119 | 508.2897 | |
| 242 | — | (Neat) | HRMS-ESI | ¹H NMR (400 MHz, CDCl₃) δ 4.75 (dd, J = 6.7, |
| | | 2939, | (m/z) | 3.4 Hz, 1H), 4.61 (dq, J = 7.9, 6.3 Hz, |
| | | 1735, | [M + Na]⁺ | 1H), 3.98 (qd, J = 4.7, 3.4 Hz, 1H), 3.44 (s, |
| | | 1686, | calcd for | 3H), 3.34-3.25 (m, 1H), 3.01 (t, J = 7.8 Hz, |
| | | 1366, | C₂₆H₄₅NO₈Na, | 1H), 2.15-2.00 (m, 1H), 1.98-1.86 (m, |
| | | 1108 | 522.3037; | 1H), 1.72-1.50 (m, 10H), 1.49-1.40 (m, |
| | | | found, | 2H), 1.43 (s, 18H), 1.39-1.29 (m, 2H), |
| | | | 522.3010 | 1.32 (d, J = 6.3 Hz, 3H) |
| 243 | — | (Neat) | HRMS-ESI | ¹H NMR (400 MHz, CDCl₃) δ 4.80 (dd, J = 6.9, |
| | | 2940, | (m/z) | 3.3 Hz, 1H), 4.67 (dq, J = 7.3, 6.4 Hz, |
| | | 1737, | [M + H]⁺ | 1H), 4.20 (dt, J = 8.4, 4.2 Hz, 1H), 3.39 (s, |
| | | 1688, | calcd for | 3H), 3.34 (t, J = 7.4 Hz, 1H), 3.22 (ddd, J = 7.6, |
| | | 1365, | C₂₆H₄₅NO₈Na, | 5.5, 2.3 Hz, 1H), 2.29-2.14 (m, 1H), |
| | | 1105 | 522.3037; | 1.97-1.87 (m, 1H), 1.81-1.59 (m, 10H), |
| | | | found, | 1.55-1.42 (m, 4H), 1.51 (s, 18H), 1.39 (d, J = 6.4 Hz, |
| | | | 522.3044 | 3H) |
| 244 | — | — | ESIMS | ¹H NMR (400 MHz, CDCl₃) δ 5.31 (s, 1H), |
| | | | m/z 414.4 | 4.99 (dq, J = 9.4, 6.3 Hz, 1H), 4.82 (t, J = 9.5 Hz, |
| | | | ([M + H]⁺) | 1H), 4.39 (s, 1H), 2.58 (hept, J = 7.0 Hz, |
| | | | | 1H), 2.21-2.04 (m, 1H), 1.91 (dq, J = 15.1, |
| | | | | 4.8 Hz, 1H), 1.57 (ddd, J = 21.2, 8.5, 4.1 Hz, |
| | | | | 4H), 1.45 (s, 10H), 1.39-1.30 (m, 1H), |
| | | | | 1.30-1.16 (m, 12H), 1.16-1.04 (m, 2H), 0.84 (t, |
| | | | | J = 7.0 Hz, 3H) |
| | | | | ¹³C NMR (101 MHz, CDCl₃) δ 176.00, |
| | | | | 172.77, 155.08, 79.60, 76.39, 73.73, 52.70, |
| | | | | 41.39, 34.34, 34.20, 28.28, 27.00, 24.76, |
| | | | | 21.51, 19.56, 18.92, 18.91, 17.94, 14.21 |
| 245 | — | — | ESIMS | ¹H NMR (400 MHz, CDCl₃) δ 5.41-5.22 (m, |
| | | | m/z 422.34 | 1H), 4.89 (dq, J = 9.2, 6.3 Hz, 1H), 4.34 (s, |
| | | | ([M + Na]⁺) | 1H), 3.39-3.22 (m, 2H), 2.91 (t, J = 9.3 Hz, |
| | | | | 1H), 2.17-1.99 (m, 1H), 1.85 (ddq, J = 19.8, |
| | | | | 13.2, 6.6 Hz, 2H), 1.62 (tdd, J = 12.9, 5.7, 3.7 Hz, |
| | | | | 1H), 1.54-1.38 (m, 14H), 1.35 (d, J = 6.3 Hz, |
| | | | | 3H), 1.27 (tdd, J = 16.2, 6.4, 2.9 Hz, |
| | | | | 2H), 1.12 (dtt, J = 19.0, 9.7, 4.0 Hz, 3H), |
| | | | | 0.92 (d, J = 6.7 Hz, 6H), 0.89 (t, J = 7.3 Hz, 3H) |
| | | | | ¹³C NMR (101 MHz, CDCl₃) δ 172.82, |
| | | | | 155.16, 84.64, 79.85, 79.56, 79.35, 75.55, |
| | | | | 52.76, 42.93, 34.37, 29.09, 28.31, 27.43, |
| | | | | 27.05, 24.97, 21.63, 20.15, 19.50, 19.46, |
| | | | | 18.51, 14.39 |
| 246 | — | (Neat) | HRMS-ESI | ¹H NMR (400 MHz, CDCl₃) δ 5.58 (s, 1H), |
| | | 3537, | (m/z) | 5.04-4.94 (m, 1H), 4.36-4.26 (m, 1H), |
| | | 3483, | [M + Na]⁺ | 3.85 (t, J = 11.5 Hz, 1H), 3.60-3.45 (m, 4H), |
| | | 3350, | calcd for | 3.45-3.36 (m, 1H), 3.36-3.26 (m, 1H), |
| | | 2931, | C₁₄H₂₅NO₇Na, | 2.52-2.39 (m, 1H), 1.84-1.73 (m, 1H), 1.44 (s, |
| | | 1728, | 342.1523; | 9H), 1.38 (d, J = 6.3 Hz, 3H), 1.34 (d, J = 1.1 Hz, |
| | | 1681, | found, | 1H) |
| | | 1520, | 342.1528 | |
| | | 1364, | | |
| | | 1159 | | |
| 247 | — | (Neat) | HRMS-ESI | ¹H NMR (400 MHz, CDCl₃) δ 7.30-7.23 (m, |
| | | 3421, | (m/z) | 2H), 7.23-7.15 (m, 2H), 6.97 (ddt, J = 8.3, |
| | | 2977, | [M + Na]⁺ | 4.7, 1.1 Hz, 3H), 6.94-6.86 (m, 1H), |
| | | 1742, | calcd for | 6.76-6.67 (m, 2H), 5.54 (d, J = 6.3 Hz, 1H), |
| | | 1707, | C₂₆H₃₃NNaO₇, | 5.31 (dq, J = 9.7, 6.3 Hz, 1H), 4.48 (dd, J = 9.6, 8.6 Hz, |
| | | 1587, | 494.2149; | 1H), 4.42-4.30 (m, 2H), 3.98-3.82 (m, |
| | | 1490, | found, | 1H), 3.76 (dd, J = 9.8, 8.1 Hz, 1H), 3.58 (dd, J = 9.8, |
| | | 1158 | 494.2157 | 1.5 Hz, 1H), 3.37 (dt, J = 12.4, 3.5 Hz, |
| | | | | 1H), 2.62-2.46 (m, 1H), 1.85 (dt, J = 14.8, |
| | | | | 4.2 Hz, 1H), 1.46 (s, 9H), 1.38 (d, J = 6.3 Hz, |
| | | | | 3H) |
| 248 | — | (Neat) | HRMS-ESI | ¹H NMR (400 MHz, CDCl₃) δ 7.34-7.22 (m, |
| | | 3370, | (m/z) | 2H), 7.06-6.94 (m, 3H), 5.49 (d, J = 6.6 Hz, |
| | | 2929, | [M + Na]⁺ | 1H), 5.14 (dq, J = 9.6, 6.4 Hz, 1H), 4.32 (q, J = 5.2 Hz, |
| | | 1750, | calcd for | 1H), 4.24 (t, J = 9.1 Hz, 1H), |
| | | 1685, | C₂₀H₂₉NNaO₇, | 3.87 (td, J = 11.4, 10.8, 1.9 Hz, 1H), 3.78 (td, J = 8.7, |
| | | 1516 | 418.1836; | 2.3 Hz, 1H), 3.68-3.58 (m, 2H), |
| | | | found, | 3.43-3.30 (m, 1H), 2.57-2.38 (m, 2H), |
| | | | 418.1848 | 1.89-1.76 (m, 1H), 1.44 (s, 9H), 1.26 (d, J = 6.4 Hz, 3H) |
| 249 | — | (Neat) | HRMS-ESI | ¹H NMR (400 MHz, CDCl₃) δ 7.31-7.20 (m, |
| | | 2976, | (m/z) | 3H), 6.93-6.88 (m, 2H), 4.89-4.76 (m, |
| | | 1742, | [M + Na]⁺ | 2H), 4.34 (ddd, J = 7.4, 6.4, 2.3 Hz, 1H), |
| | | 1703, | calcd for | 4.27-4.17 (m, 1H), 3.57 (app, J = 7.3 Hz, 1H), |
| | | 1366, | C₃₁H₄₇NNaO₈, | 2.25-2.12 (m, 1H), 2.04-1.81 (m, 2H), |
| | | 1143 | 584.3194; | 1.81-1.37 (m, 13H), 1.51 (s, 18H), 1.47 (d, J = 6.4 Hz, |
| | | | found, | 3H) |
| | | | 584.3175 | |
| 250 | — | (Neat) | ESIMS | ¹H NMR (400 MHz, CDCl₃) δ 5.94 (ddt, J = 17.0, |
| | | 2977, | m/z 580.4 | 10.3, 5.8 Hz, 1H), 5.39-5.32 (m, 1H), |
| | | 1742, | ([M + Na]⁺) | 5.29-5.23 (m, 1H), 4.89 (dd, J = 6.1, 3.9 Hz, |
| | | 1702, | | 1H), 4.80-4.71 (m, 2H), 4.64-4.59 (m, |
| | | 1366, | | 2H), 3.44 (dd, J = 8.4, 6.5 Hz, 1H), |
| | | 1250 | | 3.34-3.27 (m, 2H), 2.17-1.95 (m, 2H), |
| | | | | 1.94-1.57 (m, 7H), 1.51 (s, 18H), 1.41 (d, J = 6.3 Hz, |
| | | | | 3H), 0.87 (d, J = 6.7 Hz, 3H), 0.86 (d, J = 6.7 Hz, |
| | | | | 3H) |
| 251 | — | (Neat) | HRMS-ESI | ¹H NMR (400 MHz, CDCl₃) δ 4.89 (dd, J = 6.2, |
| | | 2975, | (m/z) | 3.9 Hz, 1H), 4.76 (qd, J = 5.8, 2.8 Hz, |
| | | 1741, | [M + Na]⁺ | 2H), 4.08 (t, J = 6.7 Hz, 2H), 3.44 (dd, J = 8.4, |
| | | 1702, | calcd for | 6.5 Hz, 1H), 3.34-3.28 (m, 2H), |
| | | 1253 | C₂₈H₄₉NNaO₁₀, | 2.18-1.95 (m, 2H), 1.93-1.55 (m, 9H), 1.51 (s, |
| | | | 582.3249; | 18H), 1.41 (d, J = 6.3 Hz, 3H), 0.96 (t, J = 7.4 Hz, |
| | | | found, | 3H), 0.87 (d, J = 6.7, 3H), 0.87 (d, J = 6.7, |
| | | | 582.3243 | 3H) |
| 252 | — | (Neat) | HRMS-ESI | ¹H NMR (400 MHz, CDCl₃) δ 7.32-7.27 (m, |
| | | 3423, | (m/z) | 2H), 7.00 (tt, J = 7.4, 1.1 Hz, 1H), |
| | | 2976, | [M + Na]⁺ | 6.96-6.91 (m, 2H), 5.54 (d, J = 6.3 Hz, 1H), 5.18 (dq, J = 9.2, |
| | | 2930, | calcd for | 6.3 Hz, 1H), 4.38-4.31 (m, 1H), |
| | | 1739, | C₂₀H₂₉NNaO₇, | 4.08 (td, J = 8.1, 7.7, 1.4 Hz, 1H), 3.89 (td, J = 12.0, |
| | | 1704, | 418.1839; | 2.2 Hz, 1H), 3.74 (t, J = 9.0 Hz, 1H), |
| | | 1492, | found, | 3.59 (dd, J = 9.6, 7.8 Hz, 1H), 3.51 (dd, J = 9.6, |
| | | 1364, | 418.1836 | 1.4 Hz, 1H), 3.27 (dt, J = 12.4, 3.5 Hz, |
| | | 1158, | | 1H), 3.03 (s, 1H), 2.56-2.45 (m, 1H), |
| | | 1042 | | 1.89-1.77 (m, 1H), 1.46-1.42 (m, 12H) |
| 253 | — | — | ESIMS | ¹H NMR (400 MHz, CDCl₃) δ 5.51 (d, J = 7.9 Hz, |
| | | | m/z 380 | 1H), 5.03 (dq, J = 9.2, 6.2 Hz, 1H), |
| | | | ([M + Na]⁺) | 4.48 (ddd, J = 8.0, 3.6, 1.7 Hz, 1H), |
| | | | | 3.90-3.76 (m, 2H), 3.54 (ddd, J = 11.4, 9.7, 3.4 Hz, 1H), |
| | | | | 3.39 (ddd, J = 11.4, 4.8, 3.6 Hz, 1H), |
| | | | | 1.71-1.00 (m, 22H), 0.88 (app t, J = 6.5 Hz, 6H) |
| | | | | ¹³C NMR (101 MHz, CDCl₃) δ 170.37, |
| | | | | 155.54, 79.74, 76.49, 69.20, 68.51, 55.36, |
| | | | | 45.51, 35.63, 30.77, 28.33, 28.30, 27.81, |
| | | | | 25.98, 22.87, 22.27, 19.88 |
| 254 | — | — | ESIMS | ¹H NMR (400 MHz, CDCl₃) δ 7.29-7.22 (m, |
| | | | m/z 436.4 | 2H), 7.03-6.91 (m, 3H), 5.72-5.57 (m, |
| | | | ([M + H]⁺) | 1H), 5.51 (d, J = 6.4 Hz, 1H), 5.15 (dq, J = 9.7, |
| | | | | 6.3 Hz, 1H), 5.11-4.98 (m, 2H), |
| | | | | 4.39-4.22 (m, 2H), 4.06-3.93 (m, 2H), |
| | | | | 3.92-3.80 (m, 1H), 3.61 (dd, J = 9.7, 8.5 Hz, 1H), |
| | | | | 3.55-3.32 (m, 3H), 2.51 (ddd, J = 15.1, 9.3, 5.7 Hz, |
| | | | | 1H), 1.86-1.76 (m, 1H), 1.45 (s, 9H), |
| | | | | 1.31 (d, J = 6.3 Hz, 3H) |
| 255 | — | — | ESIMS | ¹H NMR (400 MHz, CDCl₃) δ 7.32-7.23 (m, |
| | | | m/z 458.3 | 2H), 7.00-6.87 (m, 3H), 5.90-5.76 (m, |
| | | | ([M + Na]⁺) | 1H), 5.52 (d, J = 6.3 Hz, 1H), 5.19 (dq, J = 17.2, |
| | | | | 1.6 Hz, 1H), 5.14-5.05 (m, 2H), |
| | | | | 4.44-4.36 (m, 1H), 4.36-4.30 (m, 1H), |
| | | | | 4.24-4.17 (m, 1H), 4.17-4.10 (m, 1H), 3.93-3.81 (m, |
| | | | | 1H), 3.65 (dd, J = 9.7, 8.2 Hz, 1H), 3.47 (dd, J = 9.8, |
| | | | | 8.6 Hz, 2H), 3.36-3.25 (m, 1H), |
| | | | | 2.58-2.44 (m, 1H), 1.87-1.76 (m, 1H), 1.45 (s, |
| | | | | 9H), 1.40 (d, J = 6.3 Hz, 3H) |
| 256 | — | (Neat) | HRMS-ESI | ¹H NMR (400 MHz, CDCl₃) δ 7.30-7.23 (m, |
| | | 3368, | (m/z) | 2H), 7.03-6.90 (m, 3H), 5.51 (d, J = 6.3 Hz, |
| | | 2977, | [M + Na]⁺ | 1H), 5.15 (dq, J = 9.7, 6.3 Hz, 1H), |
| | | 1747, | calcd for | 4.37-4.29 (m, 1H), 4.23 (dd, J = 9.7, 8.5 Hz, 1H), |
| | | 1684, | C₂₁H₃₁NNaO₇, | 3.91-3.82 (m, 1H), 3.63-3.53 (m, 1H), |
| | | 1520, | 432.1993; | 3.50 (dd, J = 9.9, 1.8 Hz, 1H), 3.44-3.37 (m, 1H), |
| | | 1098 | found, | 3.33 (s, 3H), 3.31-3.24 (m, 1H), |
| | | | 432.198 | 2.58-2.42 (m, 1H), 1.87-1.74 (m, 1H), 1.45 (s, 9H), |
| | | | | 1.31 (d, J = 6.4 Hz, 3H) |
| 257 | — | — | ESIMS | ¹H NMR (400 MHz, CDCl₃) δ 5.49 (d, J = 6.3 Hz, |
| | | | m/z 450.4 | 1H), 5.03-4.93 (m, 3H), 4.88-4.83 (m, |
| | | | ([M + Na]⁺) | 2H), 4.37-4.25 (m, 2H), 4.05-3.87 (m, |
| | | | | 3H), 3.87-3.77 (m, 1H), 3.52 (dd, J = 9.7, |
| | | | | 7.8 Hz, 1H), 3.43 (dd, J = 9.7, 1.6 Hz, 1H), |
| | | | | 3.38-3.32 (m, 1H), 3.31-3.19 (m, 2H), |
| | | | | 2.47 (t, J = 13.4 Hz, 1H), 1.78 (d, J = 16.7 Hz, 1H), |
| | | | | 1.74 (t, J = 1.1 Hz, 3H), 1.72 (t, J = 1.1 Hz, |
| | | | | 3H), 1.44 (s, 9H), 1.36 (d, J = 6.3 Hz, 3H) |
| 258 | — | — | ESIMS | ¹H NMR (400 MHz, CDCl₃) δ 5.48 (d, J = 6.2 Hz, |
| | | | m/z 396.3 | 1H), 5.05 (dq, J = 9.2, 6.3 Hz, 1H), |
| | | | ([M + Na]⁺) | 4.97 (dq, J = 2.1, 1.1 Hz, 1H), 4.91 (dp, J = 3.0, 1.0 Hz, |
| | | | | 1H), 4.31 (q, J = 5.1 Hz, 1H), 4.07 (d, J = 11.8 Hz, |
| | | | | 1H), 3.89 (d, J = 12.2 Hz, 2H), |
| | | | | 3.56-3.41 (m, 3H), 3.35-3.27 (m, 1H), |
| | | | | 3.20-3.13 (m, 1H), 3.11 (d, J = 1.1 Hz, 1H), |
| | | | | 2.52-2.41 (m, 1H), 1.87-1.77 (m, 1H), 1.74 (dd, J = 1.6, |
| | | | | 0.8 Hz, 3H), 1.44 (s, 9H), 1.38 (d, J = 6.3 Hz, |
| | | | | 3H) |
| 259 | — | — | ESIMS | ¹H NMR (400 MHz, CDCl₃) δ 5.45 (d, J = 6.5 Hz, |
| | | | m/z 396.3 | 1H), 5.05-4.95 (m, 2H), 4.94-4.89 (m, |
| | | | ([M + Na]⁺) | 1H), 4.35-4.25 (m, 1H), 4.16 (d, J = 12.0 Hz, |
| | | | | 1H), 4.03 (d, J = 12.0 Hz, 1H), |
| | | | | 3.90-3.80 (m, 1H), 3.62-3.42 (m, 3H), 3.33 (ddd, |
| | | | | J = 12.2, 4.6, 3.0 Hz, 1H), 3.17 (dd, J = 9.6, |
| | | | | 8.5 Hz, 1H), 2.66 (d, J = 1.8 Hz, 1H), |
| | | | | 2.48-2.37 (m, 1H), 1.85-1.76 (m, 1H), |
| | | | | 1.77-1.74 (m, 3H), 1.44 (s, 9H), 1.39 (d, J = 6.4 Hz, 3H) |
| 260 | — | (Neat) | HRMS-ESI | ¹H NMR (400 MHz, CDCl₃) δ 7.29-7.21 (m, |
| | | 3364, | (m/z) | 2H), 7.03-6.96 (m, 2H), 6.96-6.89 (m, |
| | | 2932, | [M + Na]⁺ | 1H), 5.52 (d, J = 6.3 Hz, 1H), 5.15 (dq, J = 9.7, |
| | | 1745, | calcd for | 6.3 Hz, 1H), 4.38-4.29 (m, 1H), |
| | | 1686, | C₂₃H₃₅NNaO₇, | 4.25 (dd, J = 9.7, 8.5 Hz, 1H), 3.92-3.80 (m, 1H), |
| | | 1518, | 460.2306; | 3.64-3.56 (m, 1H), 3.52-3.43 (m, 2H), |
| | | 1492, | found, | 3.43-3.32 (m, 3H), 2.57-2.44 (m, 1H), |
| | | 1363, | 460.2293 | 1.86-1.76 (m, 1H), 1.45 (s, 9H), 1.38-1.24 (m, |
| | | 1092 | | 2H), 1.31 (d, J = 6.3 Hz, 3H), 0.68 (t, J = 7.4 Hz, |
| | | | | 3H) |
| 261 | — | — | ESIMS | ¹H NMR (400 MHz, CDCl₃) δ 7.30-7.23 (m, |
| | | | m/z 460.4 | 2H), 6.97-6.89 (m, 3H), 5.53 (d, J = 6.2 Hz, |
| | | | ([M + Na]⁺) | 1H), 5.07 (dq, J = 9.8, 6.3 Hz, 1H), |
| | | | | 4.38-4.30 (m, 1H), 4.22-4.13 (m, 1H), |
| | | | | 3.90-3.79 (m, 2H), 3.65 (dd, J = 9.7, 8.1 Hz, 1H), |
| | | | | 3.57-3.48 (m, 1H), 3.48-3.43 (m, 1H), 3.39 (dd, J = 9.8, |
| | | | | 8.7 Hz, 1H), 3.35-3.27 (m, 1H), |
| | | | | 2.58-2.43 (m, 1H), 1.85-1.76 (m, 1H), |
| | | | | 1.53-1.42 (m, 2H), 1.44 (s, 9H), 1.40 (d, J = 6.3 Hz, |
| | | | | 3H), 0.82 (t, J = 7.4 Hz, 3H) |
| | | | | ¹³C NMR (101 MHz, CDCl₃) δ 172.02, |
| | | | | 157.59, 155.15, 129.53, 121.07, 115.53, |
| | | | | 83.59, 80.15, 79.86, 75.63, 71.34, 68.20, |
| | | | | 65.05, 50.95, 28.98, 28.34, 23.40, 18.44, |
| | | | | 10.62 |
| 262 | — | (Neat) | HRMS-ESI | ¹H NMR (400 MHz, CDCl₃) δ 5.51 (d, J = 6.2 Hz, |
| | | 3363, | (m/z) | 1H), 4.92 (dq, J = 9.0, 6.4 Hz, 1H), |
| | | 2954, | [M + Na]⁺ | 4.29 (dq, J = 6.4, 4.0 Hz, 1H), 3.89-3.77 (m, 1H), |
| | | 1746, | calcd for | 3.68 (dd, J = 8.2, 6.0 Hz, 1H), 3.48 (dd, J = 9.6, |
| | | 1685, | C₂₂H₄₁NNaO₇, | 7.4 Hz, 1H), 3.42-3.27 (m, 4H), |
| | | 1516, | 454.2775; | 3.27-3.19 (m, 1H), 3.19-3.03 (m, 2H), |
| | | 1362, | found, | 2.55-2.33 (m, 1H), 1.90-1.69 (m, 3H), 1.44 (s, 9H), |
| | | 1092, | 454.2773 | 1.34 (d, J = 6.5 Hz, 3H), 0.97-0.83 (m, 12H) |
| | | 1067 | | |
| 263 | — | (Neat) | HRMS-ESI | ¹H NMR (400 MHz, CDCl₃) δ 7.30-7.19 (m, |
| | | 3379, | (m/z) | 2H), 7.04-6.98 (m, 2H), 6.98-6.87 (m, |
| | | 2871, | [M + Na]⁺ | 1H), 5.52 (d, J = 6.3 Hz, 1H), 5.16 (dq, J = 9.6, |
| | | 1757, | calcd for | 6.3 Hz, 1H), 4.77-4.73 (m, 2H), |
| | | 1692, | C₂₄H₃₅NNaO₇, | 4.37-4.26 (m, 2H), 3.96-3.81 (m, 3H), 3.63 (dd, J = 9.8, |
| | | 1518, | 472.2306; | 8.3 Hz, 1H), 3.52 (dd, J = 9.7, 1.8 Hz, |
| | | 1493, | found, | 1H), 3.47-3.35 (m, 2H), 2.52 (ddd, J = 15.0, |
| | | 1158, | 472.2298 | 9.4, 5.7 Hz, 1H), 1.87-1.76 (m, 1H), 1.50 (s, |
| | | 1093 | | 3H), 1.45 (s, 9H), 1.30 (d, J = 6.4 Hz, 3H) |
| 264 | — | — | ESIMS | ¹H NMR (300 MHz, CDCl₃) δ 7.32-7.20 (m, |
| | | | m/z 472.4 | 2H), 7.01-6.84 (m, 3H), 5.53 (d, J = 6.2 Hz, |
| | | | ([M + Na]⁺) | 1H), 5.11 (dq, J = 9.6, 6.3 Hz, 1H), |
| | | | | 4.93-4.82 (m, 1H), 4.85-4.74 (m, 1H), |
| | | | | 4.40-4.29 (m, 2H), 4.29-4.17 (m, 1H), 4.00 (d, J = 11.3 Hz, |
| | | | | 1H), 3.93-3.77 (m, 1H), 3.66 (dd, J = 9.7, |
| | | | | 8.1 Hz, 1H), 3.53-3.40 (m, 2H), |
| | | | | 3.31 (d, J = 12.3, 3.5 Hz, 1H), 2.62-2.42 (m, 1H), |
| | | | | 1.89-1.73 (m, 1H), 1.68-1.63 (m, 3H), |
| | | | | 1.45 (s, 9H), 1.41 (d, J = 6.3 Hz, 3H) |
| | | | | ¹³C NMR (75 MHz, CDCl₃) δ 172.20, 157.55, |
| | | | | 155.37, 142.36, 129.77, 121.37, 115.76, |
| | | | | 112.66, 83.67, 80.43, 80.10, 78.07, 71.43, |
| | | | | 68.30, 65.29, 51.19, 29.22, 28.56, 20.05, |
| | | | | 18.75 |
| 265 | — | (Neat) | ESIMS | ¹H NMR (300 MHz, CDCl₃) δ 7.30-7.19 (m, |
| | | 3384, | m/z 488.4 | 2H), 6.98-6.87 (m, 3H), 5.51 (d, J = 6.5 Hz, |
| | | 2975, | ([M + Na]⁺) | 1H), 5.17 (dq, J = 12.8, 6.5 Hz, 1H), 5.03 (td, |
| | | 1752, | | J = 8.4, 1.8 Hz, 1H), 4.54-4.43 (m, 1H), |
| | | 1729, | | 4.40-4.26 (m, 1H), 3.85 (ddd, J = 12.5, 10.7, |
| | | 1692, | | 2.1 Hz, 1H), 3.76-3.62 (m, 1H), |
| | | 1156 | | 3.51-3.35 (m, 2H), 2.55-2.37 (m, 1H), 2.14 (h, J = 7.0 Hz, |
| | | | | 1H), 1.90-1.75 (m, 1H), 1.43 (s, 9H), |
| | | | | 1.32 (d, J = 6.4 Hz, 3H), 0.90 (d, J = 7.0 Hz, |
| | | | | 3H), 0.82 (d, J = 7.0 Hz, 3H) |
| 266 | — | (Neat) | ESIMS | ¹H NMR (400 MHz, CDCl₃) δ 7.30-7.20 (m, |
| | | 3364, | m/z 474.4 | 2H), 7.03-6.95 (m, 2H), 6.95-6.89 (m, |
| | | 2931, | ([M + Na]⁺) | 1H), 5.53 (d, J = 6.3 Hz, 1H), 5.15 (dq, J = 9.6, |
| | | 1745, | | 6.3 Hz, 1H), 4.38-4.29 (m, 1H), |
| | | 1684, | | 4.25 (dd, J = 9.7, 8.5 Hz, 1H), 3.93-3.77 (m, 1H), |
| | | 1519, | | 3.60 (dd, J = 9.7, 8.4 Hz, 1H), 3.49 (dd, J = 9.5, |
| | | 1155 | | 1.8 Hz, 1H), 3.42-3.31 (m, 2H), |
| | | | | 3.27 (dd, J = 8.9, 6.4 Hz, 1H), 3.21 (dd, J = 8.9, 6.4 Hz, |
| | | | | 1H), 2.57-2.45 (m, 1H), 1.87-1.77 (m, |
| | | | | 1H), 1.56 (hept, J = 6.6 Hz, 1H), 1.45 (s, 9H), |
| | | | | 1.30 (d, J = 6.3 Hz, 3H), 0.69 (d, J = 6.7 Hz, |
| | | | | 3H), 0.65 (d, J = 6.7 Hz, 3H) |
| 267 | — | — | ESIMS | ¹H NMR (400 MHz, CDCl₃) δ 7.31-7.23 (m, |
| | | | m/z 474.4 | 2H), 6.97-6.87 (m, 3H), 5.53 (d, J = 6.3 Hz, |
| | | | ([M + Na]⁺) | 1H), 5.08 (dq, J = 9.8, 6.3 Hz, 1H), |
| | | | | 4.38-4.30 (m, 1H), 4.21-4.14 (m, 1H), |
| | | | | 3.91-3.80 (m, 1H), 3.66 (ddd, J = 14.1, 9.0, 7.2 Hz, 2H), |
| | | | | 3.52-3.26 (m, 4H), 2.57-2.45 (m, 1H), |
| | | | | 1.86-1.77 (m, 1H), 1.77-1.65 (m, 1H), 1.45 (s, |
| | | | | 9H), 1.40 (d, J = 6.3 Hz, 3H), 0.82 (d, J = 6.7 Hz, |
| | | | | 3H), 0.78 (d, J = 6.7 Hz, 3H) |
| | | | | ¹³C NMR (101 MHz, CDCl₃) δ 172.02, |
| | | | | 157.48, 155.15, 129.51, 121.02, 115.48, |
| | | | | 83.43, 80.61, 80.05, 79.86, 71.39, 68.17, |
| | | | | 65.05, 50.96, 28.99, 28.34, 19.44, 19.37, |
| | | | | 18.48 |
| 268 | — | — | ESIMS | — |
| | | | m/z 482.5 | |
| | | | ([M + Na]⁺) | |
| 269 | — | — | ESIMS | ¹H NMR (400 MHz, CDCl₃) δ 5.31 (d, J = 6.5 Hz, |
| | | | m/z 406.4 | 1H), 4.83 (dq, J = 9.1, 6.2 Hz, 1H), |
| | | | ([M + Na]⁺) | 4.34 (s, 1H), 3.27 (q, J = 8.7 Hz, 1H), 2.08 (s, 1H), |
| | | | | 1.89 (dp, J = 15.8, 4.9 Hz, 2H), 1.71 (ddt, J = 26.1, |
| | | | | 11.2, 6.4 Hz, 3H), 1.64-1.56 (m, 3H), |
| | | | | 1.51 (ddd, J = 11.1, 8.2, 5.5 Hz, 6H), 1.45 (s, |
| | | | | 9H), 1.38 (d, J = 6.2 Hz, 3H), 1.33-1.24 (m, |
| | | | | 2H), 1.24-1.15 (m, 1H), 1.06 (dtd, J = 28.3, |
| | | | | 8.2, 4.2 Hz, 3H) |
| | | | | ¹³C NMR (101 MHz, CDCl₃) δ 173.18, |
| | | | | 155.18, 79.75, 75.69, 52.62, 43.37, 39.09, |
| | | | | 37.31, 33.81, 32.33, 28.35, 27.29, 26.86, |
| | | | | 25.12, 25.07, 24.67, 21.94, 18.74 |
| 270 | — | — | ESIMS | ¹H NMR (400 MHz, CDCl₃) δ 7.30-7.23 (m, |
| | | | m/z 482.4 | 2H), 6.96-6.87 (m, 3H), 5.31 (d, J = 6.6 Hz, |
| | | | ([M + Na]⁺) | 1H), 5.10 (dq, J = 9.2, 6.3 Hz, 1H), 4.39 (s, |
| | | | | 1H), 4.07 (t, J = 8.9 Hz, 1H), 2.08 (d, J = 9.4 Hz, |
| | | | | 1H), 1.90 (dq, J = 21.5, 7.5, 6.9 Hz, 2H), |
| | | | | 1.74-1.59 (m, 4H), 1.56-1.47 (m, 5H), |
| | | | | 1.45 (s, 9H), 1.43 (t, J = 3.4 Hz, 2H), |
| | | | | 1.35-1.27 (m, 2H), 1.27-1.24 (m, 3H), 1.23-1.11 (m, |
| | | | | 2H), 1.03-0.91 (m, 2H) |
| | | | | ¹³C NMR (101 MHz, CDCl₃) δ 172.86, |
| | | | | 159.61, 129.56, 120.81, 115.39, 82.56, 75.17, |
| | | | | 52.96, 41.85, 37.58, 33.52, 32.11, 28.35, |
| | | | | 25.09, 25.03, 18.74 |
| 271 | — | — | ESIMS | ¹H NMR (400 MHz, CDCl₃) δ 5.89 (ddt, J = 17.2, |
| | | | m/z 446.4 | 10.5, 5.3 Hz, 1H), 5.35-5.22 (m, 2H), |
| | | | ([M + Na]⁺) | 5.16 (dq, J = 10.4, 1.2 Hz, 1H), 4.93 (dq, J = 9.2, |
| | | | | 6.3 Hz, 1H), 4.34 (s, 1H), 4.14-3.99 (m, |
| | | | | 2H), 2.97 (t, J = 9.1 Hz, 1H), 2.04 (s, 1H), |
| | | | | 1.89 (dt, J = 14.0, 7.3 Hz, 2H), 1.75 (ddd, J = 14.4, |
| | | | | 11.0, 7.1 Hz, 1H), 1.70-1.64 (m, 1H), |
| | | | | 1.64 (s, 1H), 1.58 (ddt, J = 13.1, 9.6, 3.9 Hz, |
| | | | | 3H), 1.53-1.46 (m, 5H), 1.44 (d, J = 4.0 Hz, |
| | | | | 9H), 1.42-1.38 (m, 2H), 1.36 (d, J = 6.3 Hz, |
| | | | | 3H), 1.25-1.13 (m, 2H), 1.04 (dddt, J = 16.4, |
| | | | | 12.6, 8.4, 4.7 Hz, 2H) |
| | | | | ¹³C NMR (101 MHz, CDCl₃) δ 172.90, |
| | | | | 155.18, 134.35, 116.82, 85.55, 79.69, 75.33, |
| | | | | 73.78, 52.89, 41.90, 38.63, 37.43, 33.82, |
| | | | | 32.06, 28.35, 27.48, 25.12, 25.05, 21.73, |
| | | | | 18.55 |
| 272 | — | — | ESIMS | ¹H NMR (400 MHz, CDCl₃) δ 5.36-5.22 (m, |
| | | | m/z 476.5 | 1H), 5.00 (dq, J = 9.4, 6.3 Hz, 1H), 4.79 (t, J = 9.4 Hz, |
| | | | ([M + Na]⁺) | 1H), 4.38 (s, 1H), 2.57 (hept, J = 7.0 Hz, |
| | | | | 1H), 2.14 (d, J = 26.9 Hz, 1H), 1.91 (d, J = 14.0 Hz, |
| | | | | 1H), 1.82 (dd, J = 14.3, 7.3 Hz, |
| | | | | 1H), 1.69 (ddt, J = 23.3, 12.0, 6.0 Hz, 3H), |
| | | | | 1.59-1.47 (m, 7H), 1.45 (s, 10H), |
| | | | | 1.31-1.24 (m, 1H), 1.22-1.17 (m, 9H), 1.14 (dd, J = 8.4, |
| | | | | 4.5 Hz, 2H), 1.05-0.88 (m, 3H) |
| | | | | ¹³C NMR (101 MHz, CDCl₃) δ 176.23, |
| | | | | 172.96, 73.71, 52.79, 40.66, 37.20, 34.31, |
| | | | | 33.65, 32.10, 28.35, 25.09, 25.01, 19.05, |
| | | | | 19.01, 18.02 |
| 273 | — | — | ESIMS | ¹H NMR (400 MHz, CDCl₃) δ 5.30 (d, J = 4.5 Hz, |
| | | | m/z 448.4 | 1H), 4.90 (dq, J = 9.2, 6.3 Hz, 1H), |
| | | | ([M + Na]⁺) | 4.34 (s, 1H), 3.55-3.40 (m, 2H), 2.89 (t, J = 9.2 Hz, |
| | | | | 1H), 2.13-1.99 (m, 1H), 1.96-1.82 (m, |
| | | | | 2H), 1.80-1.71 (m, 1H), 1.71-1.63 (m, |
| | | | | 2H), 1.63-1.54 (m, 6H), 1.54-1.46 (m, |
| | | | | 5H), 1.45 (s, 9H), 1.36 (d, J = 6.3 Hz, 3H), |
| | | | | 1.23-1.13 (m, 2H), 1.06 (dtt, J = 15.7, 8.1, |
| | | | | 4.1 Hz, 3H), 0.93 (t, J = 7.4 Hz, 3H) |
| | | | | ¹³C NMR (101 MHz, CDCl₃) δ 172.93, |
| | | | | 155.18, 85.35, 79.67, 75.53, 74.59, 52.88, |
| | | | | 41.98, 38.50, 37.41, 33.86, 32.06, 28.35, |
| | | | | 27.48, 25.14, 25.06, 24.74, 23.39, 21.73, |
| | | | | 18.47, 10.74 |
| 274 | — | — | ESIMS | ¹H NMR (400 MHz, CDCl₃) δ 5.29 (d, J = 7.7 Hz, |
| | | | m/z 420.4 | 1H), 4.88 (dq, J = 9.1, 6.3 Hz, 1H), |
| | | | ([M + Na]⁺) | 4.33 (s, 1H), 3.43 (s, 3H), 2.82 (t, J = 9.1 Hz, 1H), |
| | | | | 2.05 (d, J = 4.3 Hz, 1H), 1.88 (dt, J = 16.1, |
| | | | | 8.1 Hz, 2H), 1.82-1.64 (m, 3H), 1.59 (ddd, J = 12.1, |
| | | | | 7.0, 3.2 Hz, 3H), 1.55-1.46 (m, 5H), |
| | | | | 1.45 (s, 9H), 1.37 (d, J = 6.3 Hz, 3H), |
| | | | | 1.19 (ddt, J = 20.9, 11.2, 6.3 Hz, 3H), |
| | | | | 1.12-0.97 (m, 3H) |
| | | | | ¹³C NMR (101 MHz, CDCl₃) δ 172.95, |
| | | | | 155.18, 87.19, 79.69, 75.20, 60.32, 52.78, |
| | | | | 42.04, 38.48, 37.33, 33.86, 32.05, 28.34, |
| | | | | 27.33, 27.13, 25.16, 25.07, 24.68, 21.88, |
| | | | | 18.43 |
| 275 | — | — | ESIMS | ¹H NMR (400 MHz, CDCl₃) δ 7.30-7.24 (m, |
| | | | m/z 484.5 | 2H), 7.17 (dd, J = 8.4, 6.3 Hz, 1H), |
| | | | ([M + Na]⁺) | 7.14-7.09 (m, 2H), 5.32-5.21 (m, 1H), 5.07 (dq, J = 9.5, |
| | | | | 6.2 Hz, 1H), 4.94 (t, J = 9.5 Hz, 1H), |
| | | | | 4.38 (s, 1H), 2.77 (dd, J = 13.6, 3.8 Hz, 1H), |
| | | | | 2.55 (hept, J = 7.0 Hz, 1H), 2.21 (dd, J = 14.2, |
| | | | | 10.4 Hz, 1H), 2.07 (dd, J = 15.1, 8.9 Hz, 1H), |
| | | | | 1.92-1.79 (m, 2H), 1.59-1.48 (m, 1H), |
| | | | | 1.44 (s, 9H), 1.39-1.29 (m, 2H), 1.25 (d, J = 6.2 Hz, |
| | | | | 3H), 1.23-1.21 (m, 1H), 1.19 (d, J = 7.0 Hz, |
| | | | | 6H), 1.00-0.89 (m, 1H), 0.89-0.76 (m, |
| | | | | 1H) |
| | | | | ¹³C NMR (101 MHz, CDCl₃) δ 176.24, |
| | | | | 172.95, 155.10, 140.03, 128.95, 128.34, |
| | | | | 126.01, 79.77, 73.53, 52.65, 43.40, 38.15, |
| | | | | 34.21, 28.33, 28.29, 27.18, 26.01, 24.33, |
| | | | | 21.56, 19.10, 19.02, 18.98, 18.03 |
| 276 | — | — | ESIMS | ¹H NMR (400 MHz, CDCl₃) δ 7.35-7.26 (m, |
| | | | m/z 490.5 | 2H), 7.26-7.20 (m, 2H), 7.15 (dd, J = 8.4, |
| | | | ([M + Na]⁺) | 6.2 Hz, 1H), 7.13-7.06 (m, 2H), |
| | | | | 7.00-6.91 (m, 3H), 5.39-5.22 (m, 1H), 5.15 (dq, J = 9.2, |
| | | | | 6.3 Hz, 1H), 4.38 (s, 1H), 4.23 (t, J = 9.2 Hz, |
| | | | | 1H), 3.10 (dd, J = 13.4, 3.5 Hz, 1H), |
| | | | | 2.25-2.14 (m, 1H), 2.06 (s, 1H), 1.96 (s, 1H), |
| | | | | 1.92-1.81 (m, 1H), 1.59-1.49 (m, 1H), |
| | | | | 1.44 (s, 9H), 1.41-1.33 (m, 2H), 1.33-1.22 (m, |
| | | | | 5H), 1.00-0.83 (m, 1H) |
| | | | | ¹³C NMR (101 MHz, CDCl₃) δ 172.89, |
| | | | | 159.35, 155.16, 140.51, 129.67, 129.01, |
| | | | | 128.24, 125.84, 121.07, 115.36, 82.04, 79.80, |
| | | | | 75.02, 52.75, 45.23, 38.69, 28.35, 27.26, |
| | | | | 26.30, 24.40, 21.58, 18.75 |
| 277 | — | — | ESIMS | ¹H NMR (400 MHz, CDCl₃) δ 7.30-7.24 (m, |
| | | | m/z 430 | 2H), 6.96-6.85 (m, 3H), 5.55 (d, J = 7.9 Hz, |
| | | | ([M + Na]⁺) | 1H), 5.32 (dq, J = 9.0, 6.3 Hz, 1H), 4.54 (ddd, |
| | | | | J = 8.1, 3.3, 1.4 Hz, 1H), 4.01 (dd, J = 9.1, 7.6 Hz, |
| | | | | 1H), 3.92-3.82 (m, 2H), 3.71 (td, J = 11.7, |
| | | | | 2.4 Hz, 1H), 3.46 (dt, J = 12.0, 3.7 Hz, |
| | | | | 1H), 1.96 (ddt, J = 14.7, 11.5, 3.2 Hz, 1H), |
| | | | | 1.87-1.74 (m, 1H), 1.68-1.56 (m, 1H), |
| | | | | 1.46 (s, 9H), 1.38-1.33 (m, 1H), 1.29 (d, J = 6.3 Hz, |
| | | | | 3H), 1.15 (dt, J = 13.6, 7.3 Hz, 1H), |
| | | | | 0.89 (t, J = 7.3 Hz, 3H) |
| | | | | ¹³C NMR (101 MHz, CDCl₃) δ 169.89, |
| | | | | 159.31, 155.52, 129.58, 120.94, 115.19, |
| | | | | 82.87, 79.90, 74.45, 68.71, 68.38, 55.36, |
| | | | | 38.47, 32.42, 28.33, 25.09, 18.94, 12.74 |
| 278 | — | — | ESIMS | ¹H NMR (400 MHz, CDCl₃) δ 5.51 (d, J = 7.7 Hz, |
| | | | m/z 388 | 1H), 5.08 (dq, J = 9.0, 6.3 Hz, 1H), |
| | | | ([M + H]⁺) | 4.47 (ddd, J = 7.9, 3.5, 1.5 Hz, 1H), |
| | | | | 3.88-3.76 (m, 2H), 3.70 (ddd, J = 11.7, 10.6, 2.6 Hz, |
| | | | | 1H), 3.44-3.31 (m, 2H), 3.20 (dd, J = 8.4, |
| | | | | 6.5 Hz, 1H), 2.87 (dd, J = 9.0, 7.3 Hz, 1H), |
| | | | | 1.91-1.65 (m, 3H), 1.64-1.49 (m, 1H), |
| | | | | 1.44 (s, 9H), 1.34 (d, J = 6.3 Hz, 3H), |
| | | | | 1.31-1.10 (m, 2H), 0.97-0.87 (m, 9H) |
| | | | | ¹³C NMR (101 MHz, CDCl₃) δ 169.88, |
| | | | | 155.52, 85.67, 80.52, 79.78, 74.97, 69.29, |
| | | | | 68.89, 55.38, 38.52, 33.02, 29.12, 28.31, |
| | | | | 24.98, 19.52, 19.46, 19.04, 12.84 |
| 279 | — | — | ESIMS | ¹H NMR (400 MHz, CDCl₃) δ 7.36-7.27 (m, |
| | | | m/z 500.4 | 2H), 7.07-7.00 (m, 2H), 7.00-6.94 (m, |
| | | | ([M + H]⁺) | 1H), 6.94-6.88 (m, 2H), 6.81-6.74 (m, |
| | | | | 2H), 5.50 (d, J = 8.0 Hz, 1H), 5.38 (dq, J = 9.0, |
| | | | | 6.3 Hz, 1H), 4.51 (dd, J = 8.0, 2.7 Hz, |
| | | | | 1H), 4.07 (dd, J = 8.9, 7.7 Hz, 1H), 3.76 (s, |
| | | | | 5H), 3.11 (ddd, J = 13.4, 8.7, 4.2 Hz, 2H), |
| | | | | 2.76 (t, J = 10.7 Hz, 1H), 2.20 (dd, J = 12.5, |
| | | | | 5.3 Hz, 1H), 2.11 (dd, J = 13.3, 10.7 Hz, 1H), |
| | | | | 1.97 (t, J = 12.9 Hz, 1H), 1.44 (s, 9H), |
| | | | | 1.40 (dd, J = 7.8, 3.8 Hz, 1H), 1.31 (d, J = 6.3 Hz, |
| | | | | 3H) |
| | | | | ¹³C NMR (126 MHz, DMSO-d₆) δ 165.14, |
| | | | | 154.46, 153.11, 150.71, 127.48, 125.18, |
| | | | | 124.93, 116.39, 110.59, 109.03, 78.09, 75.16, |
| | | | | 69.35, 63.68, 50.54, 50.44, 34.05, 32.81, |
| | | | | 27.58, 23.57, 14.20 |
| 280 | — | — | ESIMS | ¹H NMR (400 MHz, CDCl₃) δ 7.30 (tt, J = 7.5, |
| | | | m/z 510.4 | 2.4 Hz, 2H), 7.08-7.02 (m, 2H), |
| | | | ([M + Na]⁺) | 7.00-6.95 (m, 1H), 6.94-6.86 (m, 4H), 5.49 (d, J = 7.9 Hz, |
| | | | | 1H), 5.37 (dq, J = 9.0, 6.3 Hz, 1H), |
| | | | | 4.52 (dd, J = 8.0, 2.4 Hz, 1H), 4.08 (dd, J = 9.0, |
| | | | | 7.3 Hz, 1H), 3.79 (dd, J = 10.4, 3.5 Hz, |
| | | | | 1H), 3.75 (dd, J = 10.3, 1.1 Hz, 1H), 3.16 (dt, |
| | | | | J = 12.0, 3.7 Hz, 1H), 3.09 (dd, J = 13.2, 4.8 Hz, |
| | | | | 1H), 2.83 (t, J = 10.4 Hz, 1H), |
| | | | | 2.33-2.21 (m, 1H), 2.17 (dd, J = 13.2, 10.2 Hz, 1H), |
| | | | | 2.07-1.90 (m, 1H), 1.45 (s, 9H), 1.40 (dd, J = 6.9, |
| | | | | 3.9 Hz, 1H), 1.31 (d, J = 6.3 Hz, 3H) |
| | | | | ¹³C NMR (126 MHz, CDCl₃) δ 169.91, |
| | | | | 162.29, 160.35, 159.06, 155.44, 135.99, |
| | | | | 130.29 (d, J = 7.7 Hz), 129.71, 121.23, |
| | | | | 115.25, 115.11 (d, J = 21.1 Hz), 82.62, 79.95, |
| | | | | 74.05, 68.67, 68.30, 55.27, 38.60, 37.57, |
| | | | | 32.44, 28.31, 18.88 |
| 281 | — | — | ESIMS | ¹H NMR (600 MHz, CDCl₃) δ 7.10 (ddd, J = 8.3, |
| | | | m/z 482.4 | 5.2, 2.5 Hz, 2H), 7.00-6.93 (m, 2H), |
| | | | ([M + H]⁺) | 5.46 (d, J = 8.0 Hz, 1H), 5.32 (dq, J = 9.3, 6.3 Hz, |
| | | | | 1H), 4.77 (dd, J = 9.3, 7.1 Hz, 1H), |
| | | | | 4.53 (d, J = 8.1 Hz, 1H), 3.77 (d, J = 2.2 Hz, 2H), |
| | | | | 3.17-3.10 (m, 1H), 2.87 (d, J = 8.5 Hz, 1H), |
| | | | | 2.86-2.79 (m, 1H), 2.55 (hept, J = 7.0 Hz, |
| | | | | 1H), 2.21-2.10 (m, 2H), 1.96-1.88 (m, |
| | | | | 1H), 1.46 (s, 9H), 1.39-1.31 (m, 1H), |
| | | | | 1.27 (d, J = 6.4 Hz, 3H), 1.19 (dd, J = 7.0, 3.5 Hz, |
| | | | | 6H) |
| | | | | ¹³C NMR (151 MHz, CDCl₃) δ 176.51, |
| | | | | 170.04, 161.42 (d, J = 244.2 Hz), 155.43, |
| | | | | 135.36 (d, J = 3.2 Hz), 130.33 (d, J = 7.7 Hz), |
| | | | | 115.26 (d, J = 21.1 Hz), 79.99, 72.68, 69.17, |
| | | | | 68.58, 55.34, 36.86, 36.71, 34.12, 31.71, |
| | | | | 28.31, 19.09, 18.84, 18.49 |
| 282 | — | — | HRMS-ESI | ¹H NMR (500 MHz, CDCl₃) δ 7.16-7.08 (m, |
| | | | (m/z) | 2H), 6.99-6.90 (m, 2H), 5.45 (d, J = 7.9 Hz, |
| | | | [M]⁺ | 1H), 5.17 (dq, J = 9.1, 6.3 Hz, 1H), 4.46 (dt, J = 8.1, |
| | | | calcd for | 2.3 Hz, 1H), 3.70 (d, J = 2.5 Hz, 2H), |
| | | | C₂₅H₃₆FNO₆, | 3.48 (dd, J = 9.8, 6.9 Hz, 1H), 3.36 (dd, J = 9.8, |
| | | | 465.2527; | 6.9 Hz, 1H), 3.28 (dd, J = 13.4, 4.4 Hz, |
| | | | found, | 1H), 3.04 (dt, J = 12.0, 3.8 Hz, 1H), 2.97 (dd, |
| | | | 465.253 | J = 9.2, 7.5 Hz, 1H), 2.66 (td, J = 11.5, 2.5 Hz, |
| | | | | 1H), 2.22-2.14 (m, 1H), 1.97 (tdt, J = 11.1, |
| | | | | 7.3, 3.3 Hz, 1H), 1.82 (ddt, J = 14.8, |
| | | | | 11.3, 3.3 Hz, 1H), 1.42 (s, 9H), 1.38 (d, J = 6.3 Hz, |
| | | | | 3H), 1.32-1.21 (m, 1H), |
| | | | | 1.14-1.03 (m, 1H), 0.61-0.50 (m, 2H), 0.25-0.17 (m, |
| | | | | 2H) |
| | | | | ¹⁹F NMR (471 MHz, CDCl₃) δ −112.44, |
| | | | | −125.45 (m) |
| 283 | — | — | ESIMS | ¹H NMR (400 MHz, CDCl₃) δ 8.11-8.03 (m, |
| | | | m/z 436.4 | 2H), 7.60 (ddt, J = 8.0, 6.9, 1.3 Hz, 1H), |
| | | | ([M + H]⁺) | 7.52-7.43 (m, 2H), 5.55 (d, J = 8.0 Hz, 1H), |
| | | | | 5.40 (dq, J = 9.3, 6.4 Hz, 1H), 4.98 (dd, J = 9.3, 7.4 Hz, |
| | | | | 1H), 4.57 (dt, J = 8.2, 2.4 Hz, 1H), |
| | | | | 3.89 (d, J = 2.5 Hz, 2H), 3.80-3.69 (m, 1H), |
| | | | | 3.48 (ddd, J = 11.8, 4.9, 3.3 Hz, 1H), |
| | | | | 2.01-1.81 (m, 2H), 1.46 (s, 9H), 1.58-1.36 (m, 2H), |
| | | | | 1.31 (d, J = 6.3 Hz, 3H), 1.25-1.12 (m, 1H), |
| | | | | 0.88 (t, J = 7.4 Hz, 3H) |
| | | | | ¹³C NMR (101 MHz, CDCl₃) δ 170.06, |
| | | | | 165.95, 155.49, 133.33, 129.72, 129.62, |
| | | | | 128.56, 79.93, 73.24, 69.17, 69.09, 55.45, |
| | | | | 37.03, 31.60, 29.28, 28.32, 24.46, 18.58, |
| | | | | 12.05 |
| 284 | — | — | ESIMS | ¹H NMR (400 MHz, CDCl₃) δ 5.57 (d, J = 8.0 Hz, |
| | | | m/z 400.3 | 1H), 5.22 (dq, J = 9.1, 6.4 Hz, 1H), |
| | | | ([M + H]⁺) | 4.71 (dd, J = 9.3, 7.7 Hz, 1H), 4.52 (dt, J = 8.1, 2.4 Hz, |
| | | | | 1H), 3.90-3.82 (m, 2H), 3.70 (ddd, J = 11.9, |
| | | | | 10.5, 2.6 Hz, 1H), 3.42 (ddd, J = 11.9, |
| | | | | 4.7, 3.4 Hz, 1H), 1.85 (ddt, J = 14.3, 10.6, 3.5 Hz, |
| | | | | 1H), 1.79-1.58 (m, 2H), 1.45 (s, 9H), |
| | | | | 1.49-1.38 (m, 1H), 1.36-1.27 (m, 1H), |
| | | | | 1.25 (d, J = 6.4 Hz, 3H), 1.15 (dp, J = 14.2, 7.3 Hz, |
| | | | | 1H), 1.01 (dtd, J = 5.3, 4.5, 2.0 Hz, 2H), |
| | | | | 0.95-0.85 (m, 2H), 0.89 (t, J = 7.4 Hz, 3H) |
| | | | | ¹³C NMR (101 MHz, CDCl₃) δ 174.30, |
| | | | | 169.94, 155.46, 79.82, 76.79, 73.14, 68.94, |
| | | | | 68.89, 55.36, 36.74, 31.36, 28.28, 24.20, |
| | | | | 18.47, 12.83, 11.90, 8.50, 8.24 |
| 285 | — | — | ESIMS | — |
| | | | m/z 350 | |
| | | | ([M + H]⁺) | |

*Cmpd. No.—Compound Number

TABLE 3
Biological Testing
Rating Scale
Rating Table for
Fungal Pathogens

| % Control | Rating |
|---|---|
| >80 | A |
| ≦80 | B |
| Not Tested | C |
| No Activity | D |
| Observed In | |
| Reported Assay | |

TABLE 4
Biological Activity - PUCCRT and SEPTTR
Disease Control in High and Low Volume Applications

| | Low Volume | | | | High Volume | | | |
|---|---|---|---|---|---|---|---|---|
| | (121.5 g/H*) | | | | (100 ppm*) | | | |
| *Cmpd. | PUCCRT* | | SEPTTR* | | PUCCRT* | | SEPTTR* | |
| No. | 1DP* | 3DC* | 1DP* | 3DC* | 1DP* | 3DC* | 1DP* | 3DC* |
| 1 | A | D | A | B | A | A | A | C |
| 2 | A | A | B | D | A | A | A | B |
| 3 | C | C | C | C | A | A | A | B |
| 4 | A | A | B | D | A | A | A | B |
| 5 | C | C | C | C | A | A | A | B |
| 6 | C | C | C | C | A | A | A | D |
| 7 | C | C | C | C | A | A | A | B |
| 8 | A | A | A | A | A | A | A | B |
| 9 | A | B | A | A | A | A | A | B |
| 10 | C | C | C | C | A | A | A | A |
| 11 | A | A | A | A | A | A | A | A |
| 12 | C | C | C | C | A | A | A | A |
| 13 | A | A | A | A | A | A | A | A |
| 14 | A | B | A | B | A | A | A | A |
| 15 | A | B | A | A | A | A | A | A |
| 16 | A | B | B | B | A | A | A | B |
| 17 | C | C | C | C | A | A | A | A |
| 18 | C | C | C | C | A | A | A | A |
| 19 | A | A | A | A | A | A | A | A |
| 20 | A | A | B | A | C | C | C | C |
| 21 | A | A | B | A | C | C | C | C |
| 22 | A | A | D | B | C | C | C | C |
| 23 | A | A | A | A | C | C | C | C |
| 24 | A | B | A | A | C | C | C | C |
| 25 | A | B | A | B | C | C | C | C |
| 26 | A | A | A | A | C | C | C | C |
| 27 | A | A | B | D | C | C | C | C |
| 28 | A | A | B | A | C | C | C | C |
| 29 | A | A | B | A | C | C | C | C |
| 30 | A | B | B | B | C | C | C | C |
| 31 | A | B | B | B | C | C | C | C |
| 32 | D | B | D | B | C | C | C | C |
| 33 | A | A | B | A | C | C | C | C |
| 34 | A | A | B | A | C | C | C | C |
| 35 | A | B | D | D | C | C | C | C |
| 36 | A | A | B | B | C | C | C | C |
| 37 | A | A | A | A | C | C | C | C |
| 38 | A | B | A | A | C | C | C | C |
| 39 | C | C | C | C | A | A | A | A |
| 40 | A | A | A | A | C | C | C | C |
| 41 | A | A | A | A | C | C | C | C |
| 42 | A | B | B | B | C | C | C | C |
| 43 | A | B | D | D | C | C | C | C |
| 44 | A | A | B | B | C | C | C | C |
| 45 | A | A | A | A | C | C | C | C |
| 46 | A | B | A | B | C | C | C | C |
| 47 | A | A | A | A | C | C | C | C |
| 48 | A | A | A | A | C | C | C | C |
| 49 | A | B | B | D | A | D | B | B |
| 50 | A | A | A | B | C | C | C | C |
| 51 | A | B | A | D | C | C | C | C |
| 52 | A | B | A | D | C | C | C | C |
| 53 | B | D | D | B | C | C | C | C |
| 54 | A | B | A | D | C | C | C | C |
| 55 | A | B | B | B | C | C | C | C |
| 56 | A | B | A | B | C | C | C | C |
| 57 | A | B | B | D | C | C | C | C |
| 58 | A | B | B | B | C | C | C | C |
| 59 | A | A | B | D | C | C | C | C |
| 60 | A | A | A | A | C | C | C | C |
| 61 | A | A | B | B | C | C | C | C |
| 62 | A | B | A | A | C | C | C | C |
| 63 | C | C | C | C | C | C | C | C |
| 64 | A | B | A | A | C | C | C | C |
| 65 | A | B | A | B | C | C | C | C |
| 66 | A | B | A | A | C | C | C | C |
| 67 | A | B | A | B | C | C | C | C |
| 68 | A | D | B | B | C | C | C | C |
| 69 | A | A | A | A | C | C | C | C |
| 70 | A | A | A | B | C | C | C | C |
| 71 | A | A | A | A | C | C | C | C |
| 72 | A | A | A | A | C | C | C | C |
| 73 | A | A | A | A | C | C | C | C |
| 74 | A | A | A | B | C | C | C | C |
| 75 | A | A | A | B | C | C | C | C |
| 76 | A | A | A | A | C | C | C | C |
| 77 | A | A | A | B | C | C | C | C |
| 78 | A | A | A | B | C | C | C | C |
| 79 | A | A | A | D | C | C | C | C |
| 80 | A | A | A | B | C | C | C | C |
| 81 | C | C | C | C | A | A | A | C |
| 82 | C | C | C | C | A | A | B | A |
| 83 | C | C | C | C | A | A | B | A |
| 84 | C | C | C | C | A | A | B | A |
| 85 | C | C | C | C | A | A | B | A |
| 86 | C | C | C | C | A | B | B | A |
| 87 | C | C | C | C | A | A | A | B |
| 88 | C | C | C | C | A | D | A | B |
| 89 | C | C | C | C | B | D | B | B |
| 90 | C | C | C | C | A | B | A | B |
| 91 | C | C | C | C | A | A | A | A |
| 92 | C | C | C | C | A | A | A | A |
| 93 | C | C | C | C | A | A | A | B |
| 94 | C | C | C | C | A | B | A | B |
| 95 | C | C | C | C | A | B | A | B |
| 96 | C | C | C | C | A | B | B | B |
| 97 | C | C | C | C | A | A | A | B |
| 98 | C | C | C | C | A | B | A | B |
| 99 | C | C | C | C | A | D | D | D |
| 100 | C | C | C | C | A | A | A | A |
| 101 | C | C | C | C | B | B | B | B |
| 102 | C | C | C | C | A | B | A | D |
| 103 | C | C | C | C | A | B | B | D |
| 104 | C | C | C | C | A | A | B | B |
| 105 | C | C | C | C | B | B | B | B |
| 106 | C | C | C | C | A | D | A | B |
| 107 | C | C | C | C | A | A | A | B |
| 108 | C | C | C | C | B | A | B | B |
| 109 | C | C | C | C | A | B | A | B |
| 110 | C | C | C | C | A | A | A | D |
| 111 | C | C | C | C | B | B | B | A |
| 112 | C | C | C | C | B | B | B | B |
| 113 | C | C | C | C | A | A | A | A |
| 114 | C | C | C | C | A | A | A | A |
| 115 | C | C | C | C | C | C | C | C |
| 116 | C | C | C | C | B | B | D | B |
| 117 | C | C | C | C | B | D | A | B |
| 118 | C | C | C | C | A | B | A | D |
| 119 | C | C | C | C | C | C | C | C |
| 120 | C | C | C | C | A | D | B | A |
| 121 | C | C | C | C | B | D | B | A |
| 122 | C | C | C | C | B | D | B | B |
| 123 | C | C | C | C | B | D | D | B |
| 124 | C | C | C | C | B | B | B | B |
| 125 | C | C | C | C | A | B | B | B |
| 126 | C | C | C | C | A | A | A | B |
| 127 | C | C | C | C | A | A | A | B |
| 128 | C | C | C | C | A | A | A | B |
| 129 | C | C | C | C | A | A | A | D |
| 130 | C | C | C | C | A | A | B | A |
| 131 | C | C | C | C | A | A | D | B |
| 132 | C | C | C | C | A | A | A | D |
| 133 | C | C | C | C | A | A | B | B |
| 134 | C | C | C | C | A | B | A | A |
| 135 | C | C | C | C | A | A | A | A |
| 136 | C | C | C | C | A | A | D | B |

*Cmpd. No.—Compound Number
*PUCCRT—Wheat Brown Rust (Puccinia trilicina)
*SEPTTR—Wheat Leaf Blotch (Zymoseptoria tritici)
*1DP—1 Day Protectant
*3DC—3 Day Curative
*g/H—Grams Per Hectare
*ppm—Parts Per Million

TABLE 5
Biological Activity - Disease Control at 100 ppm

| *Cmpd. | ALTESO* | CERCBE* | COLLLA* | ERYSCI* | ERYSGH* | ERYSGT* |
|---|---|---|---|---|---|---|
| No. | 1DP* | | | | | |
| 2 | B | A | A | B | B | D |
| 3 | A | B | B | D | B | B |
| 4 | A | B | A | D | B | B |
| 5 | B | B | B | D | D | B |
| 6 | B | A | A | D | D | B |
| 17 | A | A | C | B | B | C |
| 18 | A | A | C | B | B | C |
| 19 | A | A | C | B | B | C |
| 20 | A | A | A | B | B | C |
| 21 | A | B | A | B | B | C |
| 37 | D | A | A | D | D | C |

*Cmpd. No.—Compound Number
*ALTESO—Tomato Early Blight (Alternaria solani)
*CERCBE—Leaf Spot of Sugar Beets (Cercospora beticola)
*COLLLA—Cucumber Anthracnose (Glomerella lagenarium; Anamorph: Colletotricum lagenarium)
*ERYSCI—Powdery Mildew of Cucumber (Erysiphe cichoracearum)
*ERYSGH—Barley Powdery Mildew (Blumeria graminis f. sp. hordei; Synonym: Erysiphe graminis f. sp. hordei)
*ERYSGT—Wheat Powdery Mildew (Blumeria graminis f. sp. tritici; Synonym: Erysiphe graminis f. sp. tritici)
*1DP—1 Day Protectant

TABLE 6
Biological Activity-Disease Control at 100 ppm

| *Cmpd. | LEPTNO* | PYRIOR* | RHYNSE* | UNCINE* | VENTIN* |
|---|---|---|---|---|---|
| No. | 1DP | | | | |
| 2 | A | A | C | B | B |
| 3 | A | A | C | B | B |
| 4 | A | A | C | B | D |
| 5 | A | B | C | B | B |
| 6 | A | A | C | B | B |
| 17 | C | A | A | A | C |
| 18 | C | A | A | B | C |
| 19 | C | A | A | B | C |
| 20 | A | C | B | A | B |
| 21 | A | C | A | A | A |
| 37 | A | A | D | A | C |

*Cmpd. No.-CompoundNumber
*LEPTNO-Wheat Glume Blotch (Leptosphaeria nodorum)
*PYRIOR-Rice Blast (Magnaporthe grisea; Anamorph: Pyricularia oryzae)
*RHYNSE-Barley Scald (Rhyncosporium secalis)
*UNCINE-Grape Powdery Mildew (Uncinula necator)
*VENTIN-Apple Scab (Venturia inaequalis)
*1DP-1 Day Protectant

TABLE 7
Biological Activity-
Disease Control at 25 ppm

| *Cmpd. | PHAKPA* | |
|---|---|---|
| No. | 1DP* | 3DC* |
| 11 | A | A |
| 17 | A | B |
| 18 | A | A |
| 19 | A | B |
| 20 | B | D |
| 21 | B | D |

*Cmpd. No.-Compound Number
*PHAKPA-Asian Soybean Rust (Phakopsora pachyrhizi)
*1DP-Day Protectant
*3DC-3 Day Curative

1. A composition for the control of a fungal pathogen including mixtures of at least one of the compounds of Formula 1 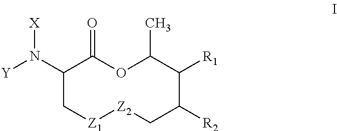
wherein
X is H or C(O)R₄;
Y is H, C(O)R₄, or Q;
Z₁ and Z₂ are independently selected from the group consisting of O and CH₂, with the provisio that Z₁ and Z₂ are not simultaneously O;
Q is 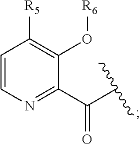
R₁ is selected from the group consisting of OR₃ and CH₂R₃;
R₂ is selected from the group consisting of OR₃, CH₂R₃, and hydrogen;
R₃ is selected from the group consisting of hydrogen, alkyl, alkenyl, aryl, Si(R₇)₃, and C(O)R₈, each optionally substituted with 0, 1, or multiple R₁₀;
R₄ is selected from the group consisting of alkyl, alkoxy, and benzyloxy, each optionally substituted with 0, 1, or multiple R₇;
R₅ is selected from the group consisting of hydrogen and alkoxy;
R₆ is selected from the group consisting of hydrogen, —C(O)R₉ and —CH₂OC(O)R₉;
R₇ is selected from the group consisting of alkyl, halo, and alkoxy;
R₈ is selected from the group consisting of alkyl, alkenyl, halo, haloalkyl, alkoxy, aryl, heteroaryl, heterocyclyl, and —C(O)R₇;
R₉ is selected from the group consisting of alkyl and alkoxy, each substituted with 0, 1, or multiple R₁₀; and
R₁₀ is selected from the group consisting of alkyl, alkenyl, halo, haloalkyl, alkoxy, aryl, heteroaryl, heterocyclyl, thioalkyl, and —C(O)R₇.

2. A composition according to claim 1, wherein X is hydrogen and Y is Q.

3. A composition according to claim 2, wherein R₅ is alkoxy.

4. A composition according to claim 3, wherein R₆ is hydrogen.

5. A composition according to claim 4, wherein R₃ is selected from the group consisting of hydrogen, alkyl, alkenyl, and aryl, each optionally substituted with 0, 1, or multiple R₁₀.

6. (canceled)

7. A composition according to claim 4, wherein R₃ is independently selected for R₁ and R₂ from the group consisting of hydrogen, alkyl, alkenyl, and aryl, each optionally substituted with 0, 1, or multiple R₁₀.

8. A composition according to claim 3, wherein R₆ is selected from the group consisting of —C(O)R₉ and —CH₂OC(O)R₉.

9. A composition according to claim 8, wherein R₉ is alkyl, optionally substituted with 0, 1, or multiple R₁₀.

10. A composition according to claim 9, wherein R₃ is selected from the group consisting of hydrogen, alkyl, alkenyl, and aryl, each optionally substituted with 0, 1, or multiple R₁₀.

11. (canceled)

12. A composition according to claim 9, wherein R₃ is independently selected for R₁ and R₂ from the group consisting of hydrogen, alkyl, alkenyl, and aryl, each optionally substituted with 0, 1, or multiple R₁₀.

13. A composition according to claim 12, wherein R₉ is selected from the group consisting of —CH₃, —CH(CH₃)₂, and —CH₂OCH₂CH₃.

14. A composition for the control of a fungal pathogen including at least one of the compositions of claim 7 and a phytologically acceptable carrier material.

15. A composition for the control of a fungal pathogen including at least one of the compositions of claim 12 and a phytologically acceptable carrier material.

16. (canceled)

17. A composition for the control of a fungal pathogen including mixtures of at least one of the compositions of claim 7 and another pesticide including fungicides, insecticides, nematocides, miticides, arthropodicides, bactericides and combinations thereof.

18. A composition for the control of a fungal pathogen including mixtures of at least one of the compositions of claim 12 and another pesticide including fungicides, insecticides, nematocides, miticides, arthropodicides, bactericides and combinations thereof.

19. (canceled)

20. (canceled)

21. (canceled)

22. (canceled)

23. (canceled)

24. A method for the control and prevention of fungal attack on a plant, the method including the step of: Applying a fungicidally effective amount of at least one of the compositions of claim 7 to at least one of the plant, an area adjacent to the plant, soil adapted to support growth of the plant, a root of the plant, and foliage of the plant.

25. A method for the control and prevention of fungal attack on a plant, the method including the step of: Applying a fungicidally effective amount of at least one of the compositions of claim 12 to at least one of the plant, an area adjacent to the plant, soil adapted to support growth of the plant, a root of the plant, and foliage of the plant.

26. (canceled)

27. (canceled)

28. (canceled)
